(19) United States
(12) Patent Application Publication
(10) Pub. No.: US 2006/0094081 A1
(43) Pub. Date: May 4, 2006

(54) Crystal structure of the c-fms kinase domain: applications and use of heterologous substitutions of kinase insert domains for crystallization

(76) Inventors: Carsten Schubert, Phoenixville, PA (US); Barry A. Springer, Wilmington (DE); Ingrid Deckman, Berwyn, PA (US); Raymond J. Patch, Yardley, PA (US); Geoffrey T. Struble, West Chester, PA (US); Hongchang Ma, Newark, DE (US); Celine Schalk-Hihi, Phoenixville, PA (US); Benjamin M. Brandt, Philadelphia, PA (US); Ioanna Petrounia, King of Prussia, PA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/255,147

(22) Filed: Oct. 20, 2005

Related U.S. Application Data
(60) Provisional application No. 60/620,698, filed on Oct. 22, 2004.

(51) Int. Cl. G06F 19/00; C07H 21/04; C12P 21/06; C12N 9/12

(52) U.S. Cl. 435/69.1; 435/194; 435/320.1; 435/325; 536/23.2; 702/19

(57) ABSTRACT

The present invention includes a crystal structure of the kinase domain of c-fms and a methodology to produce diffraction quality crystals of the c-fms kinase domain by heterologous substitution of the kinase insert domain. Also included in the invention is the structure of the c-fms kinase domain in liganded form for use in the discovery of inhibitors of c-fms for the treatment of diseases caused by inappropriate activity of c-fms. The present invention includes descriptions of the X-ray diffraction patterns of the crystals. The diffraction patterns allow the three dimensional structure of c-fms to be determined at atomic resolution so that ligand binding sites on can be identified and the interactions of ligands with amino acid residues can be modeled.

### FIELD OF THE INVENTION

The present invention generally pertains to the fields of molecular biology, protein crystallization, X-ray diffraction analysis, three-dimensional structural determination, molecular modeling and structure based rational drug design. The present invention provides crystallized peptides of the c-fms kinase domain as well as descriptions of the X-ray diffraction patterns. The X-ray diffraction patterns of the c-fms kinase domain crystals are of sufficient resolution so that the three-dimensional structure can be determined at atomic resolution, ligand binding sites on c-fms can be identified, and the interactions of ligands with c-fms amino acid residues can be modeled.

The high resolution maps provided by the present invention and the models prepared using such maps also permit the design of ligands which can function as active agents. Thus, the present invention has applications to the design of active agents which include, but are not limited to, those that find use as inhibitors of c-fms for the treatment of diseases caused by inappropriate activity of c-fms.

### BACKGROUND

Protein kinases are enzymes that serve as key components of signal transduction pathways by catalyzing the transfer of the terminal phosphate from ATP to the hydroxy group of tyrosine, serine and threonine residues of proteins. As a consequence, protein kinase inhibitors and substrates are valuable tools for assessing the physiological consequences of protein kinase activation. The overexpression or inappropriate expression of normal or mutant protein kinases in mammals has been demonstrated to play significant roles in the development of many diseases, including cancer, diabetes and autoimmune diseases.

Protein kinases can be divided into two classes: those, which preferentially phosphorylate tyrosine residues (protein tyrosine kinases) and those, which preferentially phosphorylate serine and/or threonine residues (protein serine/threonine kinases). Protein tyrosine kinases perform diverse functions ranging from stimulation of cell growth and differentiation to arrest of cell proliferation. They can be classified as either receptor protein tyrosine kinases or intracellular protein tyrosine kinases. The FMS or CSF-1-R protooncogene encodes the macrophage colony stimulating factor I receptor (or CSF-1-R or c-fms), which is the cell surface receptor for the colony stimulating factor I (CSF-1 or M-CSF) [1]. c-fms is part of the Platelet Derived Growth Factor (“PDGF”) receptor family, which includes PDGFR, the stem cell factor receptor (c-kit), c-fms, VEGFR-1 (flt-1) and VEGFR-2 (KDR).

Receptor Tyrosine Kinases (RTKs), such as c-fms, share a common architecture by which an extracellular ligand-binding domain is connected via a transmembrane segment to an intracellular catalytic domain with intrinsic tyrosine kinase activity. Binding of the ligand to the ligand-binding domain induces a conformational change, which leads in most cases to receptor dimerization, autophosphorylation of the kinase domain or adjacent domains and activation of the kinase. The activated RTK in turn trans-phosphorylates specific tyrosine residues of their respective substrates, thus transmitting the signal further. Additional members of the RTK subfamilies include the epidermal growth factor (“EGF”) family, (HER-1, HER-2/neu and HER-3 receptors), which code for oncogenes that have been have been linked to breast, colorectal and prostate cancers.

Insulin receptor (“IR”) and insulin-like growth factor I receptor (“IGF-1R”) are structurally and functionally related but exert distinct biological effects. IGF-1R over-expression has been associated with breast cancer. Fibroblast growth factor (“FGR”) receptors consist of four receptors, which are responsible for the production of blood vessels, for limb outgrowth, and for the growth and differentiation of numerous cell types.

Mononuclear phagocyte colony-stimulating factor (CSF-1 or M-CSF) is a polypeptide growth factor, which stimulates the survival, proliferation, and differentiation of haematopoietic cells of the monocyte-macrophage series. Multiple forms of soluble CSF-1 are produced by proteolytic cleavage of membrane-bound precursors, some of which are stably expressed at the cell surface [2].

Valuable insight into the signaling role of M-CSF and its receptor c-fms comes from the M-CSF deficient mice strain (op/op) [3]. These mice exhibit a selective reduction of monocytes, osteoclasts and macrophages in muscle, joints and other tissues. Furthermore these mice are osteoporotic and exhibit reduced fertility, but the incapability to produce functional M-CSF appears not to be life threatening per se. op/op mice are resistant to collagen-induced arthritis and show a reduced rate of mammary tumor progression into metastasis [4].

M-CSF has been shown to exacerbate collagen-induced arthritis in mouse models an effect, which could be suppressed with M-CSF blocking antibodies [5]. In another study M-CSF and GM-CSF (granulocyte macrophage colony-stimulating factor) induced prolonged inflammation and recruitment of macrophages in an mBSA induced arthritis model [6]. These studies demonstrated that CSF-1 and GM-CSF can exacerbate and prolong the histopathology of acute inflammatory arthritis and lend support to monocytes/macrophages being a driving influence in the pathogenesis of inflammatory arthritis. The data shown in these studies suggest that either M-CSF or its cognate receptor c-fms are suitable targets for treating arthritis or other macrophage induced inflammatory diseases.

In a recent study, expression of c-fms in various tumors was linked to poor survivability and increased tumor size [7]. M-CSF and c-fms have also been shown to be expressed by carcinomas of the breast and other epithelia of the female reproductive tract where activation of the receptor by ligand produced either by the tumor cells or by stromal elements stimulates tumor cell invasion by a urokinase-dependent mechanism [8]. These results also support other preclinical findings that CSF-1R may be involved in local invasion and metastasis. Thus, this receptor may be an effective anti-cancer target.

Recent studies indicate that macrophages, infected with HIV-1 produce high levels of M-CSF related specifically to HIV-1 and not other viral infections. High levels of M-CSF appear to be important to sustain HIV replication in vitro [9], a fact that is also corroborated by inhibition of HIV-1 replication through M-CSF scavenging agents (anti-M-CSF monoclonal or polyclonal Antibodies or soluble M-CSF receptors). These results suggest that antagonists for the action of M-CSF may represent novel a strategy for inhibiting the spread of HIV-1.

Overview of the c-fms Structures

The structure of the c-fms kinase domain closely resembles other kinase domain structures in the inactive form determined so far [12-14]. c-fms is organized in a two-lobe structure (FIG. 1). The N-lobe, comprised of 5 twisted β-sheets and a single α-helix-C is connected to the mostly α-helical C-lobe by a hinge region. The N-lobe and hinge regions are mainly responsible for nucleotide or inhibitor binding and provide part of the catalytic residues, whereas the C-lobe is responsible for substrate binding and catalysis. Nucleotide or inhibitor binding takes place in a deep cleft between the N- and C-lobe. Further important structural motifs are the glycine rich nucleotide binding loop (residues 590-594), the activation loop (residues 796-825) and the catalytic loop (residues 776-783). The native c-fms kinase insert domain (residues 680-751), which has been replaced by the FGFR kinase insert domain is located between α-helix-D and α-helix-E and is mostly disordered.

Activation Loop

The activation loop in RTKs is an essential element for the regulation of the kinase activity. In RTKs the activation loop is approximately 22 amino acids long and begins with a conserved Asp-Phe-Gly (DFG) motif and ends with a tyrosine kinase conserved Pro [15]. Autophosphorylation of tyrosines present in the activation loop has been shown to be essential for stimulation of activity for RTKs. In the absence of phosphorylation the activation loop is not properly positioned for catalysis and prevents binding of ATP. Phosphorylation events in the activation loop stabilize a conformation in which the activation loop is accessible to substrates and residues important for catalysis are positioned properly. Tyr809 is the single tyrosine present in the c-fms activation loop and is one of several that are phosphorylated in response to ligand binding. Tyr809 is bound in the active site in a manner very similar to that of Tyr1162 of the inactive form of IRK [13]. The phenol group of Tyr809 forms hydrogen-bonding interactions with Asp778 and Arg782 of the catalytic loop, which stabilize the inactive conformation of the activation loop.

The effect of its phosphorylation on this critical residue in the activation loop has been established by several mutagenesis studies. For instance a Tyr809Phe mutation prevents differentiation of macrophage colony-stimulating factor (M-CSF)-dependent bone marrow macrophages into osteoclasts [16]. In a rat cell line model Tyr809Gly abolished kinase activity and Tyr809Phe reduced kinase activity by 40-60% [17]. In a previous study the same Tyr809Phe mutation was shown to retain activity as a tyrosine kinase in vitro and in vivo, was able to undergo CSF-1-dependent association with a phosphatidylinositol 3-kinase, and induced expression of the protooncogenes c-fos and junB, underscoring its ability to trigger some of the known cellular responses to CSF-1 [18]. On the other hand the mutated receptor failed to induce mitogenesis.

Juxtamembrane Domain

The c-fms juxtamembrane domain (JM-domain) corresponds to residues 538-572 and contains two tyrosines (546 and 561). Tyr546 was shown to be a major autophosphorylation site and binds to a yet unidentified 55 kDa phosphoprotein [19]. A phosphopeptide modeled on the sequence of Tyr561 and surrounding residues competed with the association of Fyn with c-fms [20]. Furthermore, mutational analysis demonstrated that this and other sequences were required for the efficient association of Src family kinases with activated c-fms in vivo.

Structurally, the JM-domain adopts a similar arrangement as the one observed in the recently determined flt3 kinase structure [21]. Residues 548-552 are wedged between the catalytically important α-helix C and a β-sheet like loop region (residues 772-776) just preceding the catalytic loop. Residues N-terminal to Val548 do not show any electron density and are disordered. Parts of the JM-domain (558-559 and 565-572) are also disordered; this is in contrast to the flt3 structure in which the whole JM-domain was traced.

Another difference is the main anchoring point of the JM-domain to the bulk of the kinase domain. In c-fms Trp550 serves as the main anchor and is wedged deep into a cleft under α-helix C, whereas in flt3 the anchoring residue is Tyr572, which is located 2 residues upstream along the JM-domain. W550 sits in a hydrophobic pocket formed by (Ile636, Met637, Leu640, Ile646, Leu769, Cys774 and Ile794). It also forms a π face-to-edge interaction with His776. The backbone amid forms a hydrogen bond with one of the carboxyl atoms of Asp796, which is part of the DFG motif and signifies the start of the activation loop. The backbone oxygen of Trp550 also forms another hydrogen bond with the side chains of Arg777. The extended network of hydrophobic interactions and the backbone hydrogen-bonding network keep W550 firmly seated in its place, a fact underscored by the significantly lower B-factors of Trp550 as compared to its neighboring residues. Downstream of W550 a 3 residue antiparallel β-sheet like interaction between residues 551-553 and 773-775 provides additional anchorage for the JM-domain. This structural organization is also similar to that adopted by the activation loop of activated Insulin Receptor Kinase (IRK-A) in complex with AMP-PNP [22]. In IRK-A the activation loop is displaced from its inhibitory position in the nucleotide-binding pocket and folds partly around the kinase-domain parallel to the interface between the N- and C-lobe. Residues 1153 and 1157 are also wedged under the α-helix C and form a similar β-sheet like interaction as the c-fms JM-domain.

Kinase Insert Domain (KID)

The kinase insert domain is an additional loop region found in a subset of RTKs, which is located between α-helix D and α-helix E. It can vary in length from a dozen to almost 100 residues. There are several reports that the KID is involved in downstream signaling of c-fms through the mediation of protein-protein interactions. Deletion of the entire kinase insert domain completely abrogated signal transduction by the CSF-1 receptor expressed in Rat-2 fibroblasts [23]. Mutation of either Tyr697 or Tyr721 (Tyr699 and Tyr723 in human c-fms) compromised signal transduction by c-fms and the receptor lost all ability to induce changes in morphology or to increase cell growth rate in response to CSF-1. Early protein constructs, which utilized the full KID did not yield any crystals.

A need continues to exist for the development of modeling systems to design and select potent, small molecules that are inhibitors of c-fms for the treatment of diseases caused by inappropriate activity of c-fms.

### SUMMARY OF THE INVENTION

The present invention includes an isolated chimeric kinase receptor polypeptide, wherein the polypeptide comprises an ATP binding pocket linked to a substrate binding pocket by a kinase insert domain (KID), wherein the domain is heterologous to the ATP binding pocket or the substrate binding pocket. The invention also includes a crystal comprising the chimeric kinase receptor polypeptide and a crystal comprising a fragment of the chimeric kinase receptor polypeptide. In one aspect of the invention, the ATP binding pocket and substrate binding pocket are c-fms. In a different aspect of the invention, the heterologous KID is selected from the group consisting of FGFR1, tie2 and IRK.

In one aspect of the invention, the invention includes an isolated chimeric kinase receptor polypeptide, wherein the chimeric polypeptide comprises an amino acid sequence beginning at c-fms amino acid position 538 and continuing through c-fms amino acid position 922 wherein the native c-fms KID is replaced with a KID sequence comprising a heterologous KID amino acid sequence beginning at c-fms amino acid positions 672-688, SEQ ID NO. 1. The invention also includes a crystal comprising the polypeptide or a crystal comprising a fragment of the polypeptide. In one embodiment, the heterologous KID is selected from the group consisting of FGFR1, tie2 and IRK. In another aspect of the invention, the chimeric polypeptide has an amino acid sequence having at least 95% amino acid sequence identity to a sequence selected from the group consisting of SEQ ID NO. 2 (FMS/FGFR1 chimera); SEQ ID NO. 4 (FMS/tie chimera) and SEQ ID NO: 6 (FMS/irk chimera). The chimeric polypeptide or the chimeric kinase receptor polypeptide can be in crystalline form.

The invention also includes a crystal comprising a chimeric kinase receptor polypeptide wherein the polypeptide comprises an ATP binding pocket linked to a substrate binding pocket by a kinase insert domain wherein the domain is heterologous to the ATP binding pocket or the substrate binding pocket, or a fragment thereof, wherein the crystal comprises a crystal structure defined by one or more structure coordinates of c-fms amino acid residues Trp 550, Lys 586, Thr 587, Leu 588, Gly 589, Val 596, Glu 598, Ala 614, Val 615, Lys 616, Glu 633, Met 637, Leu 640, Ile 646, Val 647, Val 661, Thr 663, Glu 664, Tyr 665, Cys 666, Cys 667, Tyr 668, Gly 669, Asp 670, Asn 673, Arg 677, Cys 774, Ile 775, His 776, Arg 782, Asn 783, Leu 785, Ile 794, Gly 795, Asp 796, Phe 797, Gly 798, Leu 799, Ala 800, Arg 801, Asp 802 according to Tables 1, 2 or 3 or similar structure coordinates for said amino acids comprising a root mean square deviation of non-hydrogen atoms of less than about 1.5 Å when superimposed on the non-hydrogen atom positions of the corresponding atomic coordinates of Tables 1, 2 or 3

In a different aspect the crystal comprises a chimeric kinase receptor polypeptide wherein the polypeptide comprises an ATP binding pocket linked to a substrate binding pocket by a kinase insert domain wherein the domain is heterologous to the ATP binding pocket or the substrate binding pocket, or a fragment thereof, wherein the crystal comprises a crystal structure defined by one or more structure coordinates of c-fms amino acid residues Trp 550, Lys 586, Thr 587, Leu 588, Gly 589, Val 596, Glu 598, Ala 614, Val 615, Lys 616, Glu 633, Met 637, Leu 640, Ile 646, Val 647, Val 661, Thr 663, Glu 664, Tyr 665, Cys 666, Cys 667, Tyr 668, Gly 669, Asp 670, Asn 673, Arg 677, Cys 774, Ile 775, His 776, Arg 782, Asn 783, Leu 785, Ile 794, Gly 795, Asp 796, Phe 797, Gly 798, Leu 799, Ala 800, Arg 801, Asp 802 according to Tables 1, 2 or 3 or similar structure coordinates for said amino acids comprising a root mean square deviation of non-hydrogen atoms of less than about 0.75 Å when superimposed on the non-hydrogen atom positions of the corresponding atomic coordinates of Tables 1, 2 or 3.

In a different aspect, the crystal comprises a chimeric kinase receptor polypeptide wherein the polypeptide comprises an ATP binding pocket linked to a substrate binding pocket by a kinase insert domain wherein the domain is heterologous to the ATP binding pocket or the substrate binding pocket, or a fragment thereof, wherein the crystal comprises a crystal structure defined by one or more structure coordinates of c-fms amino acid residues Lys 586, Leu 588, Gly 589, Val 596, Glu 598, Ala 614, Lys 616, Val 647, Thr 663, Glu 664, Tyr 665, Cys 666, Cys 667, Tyr 668, Gly 669, Asp 670, Arg 677, Arg 782, Leu 785, Asp 796, Phe 797, Gly 798, Leu 799, Ala 800, Arg 801, Asp 802 according to Tables 1, 2 or 3 or similar structure coordinates for said amino acids comprising a root mean square deviation of non-hydrogen atoms of less than about 1.5 Å when superimposed on the non-hydrogen atom positions of the corresponding atomic coordinates of Tables 1, 2 or 3.

The invention also includes a crystal comprising a chimeric kinase receptor polypeptide wherein the polypeptide comprises an ATP binding pocket linked to a substrate binding pocket by a kinase insert domain wherein the domain is heterologous to the ATP binding pocket or the substrate binding pocket, or a fragment thereof, wherein the crystal comprises a crystal structure defined by one or more structure coordinates of c-fms amino acid residues Lys 586, Leu 588, Gly 589, Val 596, Glu 598, Ala 614, Lys 616, Val 647, Thr 663, Glu 664, Tyr 665, Cys 666, Cys 667, Tyr 668, Gly 669, Asp 670, Arg 677, Arg 782, Leu 785, Asp 796, Phe 797, Gly 798, Leu 799, Ala 800, Arg 801, Asp 802 according to Tables 1, 2 or 3 or similar structure coordinates for said amino acids comprising a root mean square deviation of non-hydrogen atoms of less than about 0.75 Å when superimposed on the non-hydrogen atom positions of the corresponding atomic coordinates of Tables 1, 2 or 3.

The invention also includes a crystal comprising a chimeric kinase receptor polypeptide wherein the polypeptide comprises an ATP binding pocket linked to a substrate binding pocket by a kinase insert domain wherein the domain is heterologous to the ATP binding pocket or the substrate binding pocket, or a fragment thereof, wherein the crystal comprises a crystal structure defined by structure coordinates of c-fms amino acid residues Lys 586, Leu 588, Gly 589, Val 596, Glu 598, Ala 614, Lys 616, Val 647, Thr 663, Glu 664, Tyr 665, Cys 666, Cys 667, Tyr 668, Gly 669, Asp 670, Arg 782, Asn 783, Leu 785, Asp 796, Phe 797, Leu 799, Ala 800, Arg 801 according to Tables 1, 2 or 3 or similar structure coordinates for said amino acids comprising a root mean square deviation of non-hydrogen atoms of less than about 1.5 Å when superimposed on the non-hydrogen atom positions of the corresponding atomic coordinates of Tables 1, 2 or 3.

The invention also includes a crystal comprising a chimeric kinase receptor polypeptide wherein the polypeptide comprises an ATP binding pocket linked to a substrate binding pocket by a kinase insert domain wherein the domain is heterologus to the ATP binding pocket or the substrate binding pocket, or a fragment thereof, wherein the crystal comprises a crystal structure defined by structure coordinates of c-fms amino acid residues Lys 586, Leu 588, Gly 589, Val 596, Glu 598, Ala 614, Lys 616, Val 647, Thr 663, Glu 664, Tyr 665, Cys 666, Cys 667, Tyr 668, Gly 669, Asp 670, Arg 782, Asn 783, Leu 785, Asp 796, Phe 797, Leu 799, Ala 800, Arg 801 according to Tables 1, 2 or 3 or similar structure coordinates for said amino acids comprising a root mean square deviation of non-hydrogen atoms of less than about 0.75 Å when superimposed on the non-hydrogen atom positions of the corresponding atomic coordinates of Tables 1, 2 or 3.

In another aspect of the invention, the invention comprises a crystal comprising a chimeric kinase receptor polypeptide, wherein said chimeric polypeptide comprises an amino acid sequence beginning at c-fms amino acid position 538 and continuing through c-fms amino acid position 922 wherein the native c-fms KID is replaced with a KID sequence comprising a heterologous KID amino acid sequence beginning at c-fms amino acid positions 672-688, or a fragment of the chimeric kinase receptor polypeptide, wherein said crystal comprises a crystal structure defined by one or more structure coordinates of c-fms amino acid residues Trp 550, Lys 586, Thr 587, Leu 588, Gly 589, Val 596, Glu 598, Ala 614, Val 615, Lys 616, Glu 633, Met 637, Leu 640, Ile 646, Val 647, Val 661, Thr 663, Glu 664, Tyr 665, Cys 666, Cys 667, Tyr 668, Gly 669, Asp 670, Asn 673, Arg 677, Cys 774, Ile 775, His 776, Arg 782, Asn 783, Leu 785, Ile 794, Gly 795, Asp 796, Phe 797, Gly 798, Leu 799, Ala 800, Arg 801, Asp 802 according to Tables 1, 2 or 3 or similar structure coordinates for said amino acids comprising a root mean square deviation of non-hydrogen atoms of less than about 1.5 Å when superimposed on the non-hydrogen atom positions of the corresponding atomic coordinates of Tables 1, 2 or 3.

In another aspect of the invention, the invention comprises a crystal comprising a chimeric kinase receptor polypeptide, wherein said chimeric polypeptide comprises an amino acid sequence beginning at c-fms amino acid position 538 and continuing through c-fms amino acid position 922 wherein the native c-fms KID is replaced with a KID sequence comprising a heterologous KID amino acid sequence beginning at c-fms amino acid positions 672-688, or a fragment of the chimeric kinase receptor polypeptide, wherein said crystal comprises a crystal structure defined by one or more structure coordinates of c-fms amino acid residues Trp 550, Lys 586, Thr 587, Leu 588, Gly 589, Val 596, Glu 598, Ala 614, Val 615, Lys 616, Glu 633, Met 637, Leu 640, Ile 646, Val 647, Val 661, Thr 663, Glu 664, Tyr 665, Cys 666, Cys 667, Tyr 668, Gly 669, Asp 670, Asn 673, Arg 677, Cys 774, Ile 775, His 776, Arg 782, Asn 783, Leu 785, Ile 794, Gly 795, Asp 796, Phe 797, Gly 798, Leu 799, Ala 800, Arg 801, Asp 802 according to Tables 1, 2 or 3 or similar structure coordinates for said amino acids comprising a root mean square deviation of non-hydrogen atoms of less than about 0.75 Å when superimposed on the non-hydrogen atom positions of the corresponding atomic coordinates of Tables 1, 2 or 3.

In another aspect of the invention, the invention comprises a crystal comprising a chimeric kinase receptor polypeptide, wherein said chimeric polypeptide comprises an amino acid sequence beginning at c-fms amino acid position 538 and continuing through c-fms amino acid position 922 wherein the native c-fms KID is replaced with a KID sequence comprising a heterologous KID amino acid sequence beginning at c-fms amino acid positions 672-688, or a fragment of the chimeric kinase receptor polypeptide, wherein said crystal comprises a crystal structure defined by one or more structure coordinates of c-fms amino acid residues Lys 586, Leu 588, Gly 589, Val 596, Glu 598, Ala 614, Lys 616, Val 647, Thr 663, Glu 664, Tyr 665, Cys 666, Cys 667, Tyr 668, Gly 669, Asp 670, Arg 677, Arg 782, Leu 785, Asp 796, Phe 797, Gly 798, Leu 799, Ala 800, Arg 801, Asp 802 according to Tables 1, 2 or 3 or similar structure coordinates for said amino acids comprising a root mean square deviation of non-hydrogen atoms of less than about 1.5 Å when superimposed on the non-hydrogen atom positions of the corresponding atomic coordinates of Tables 1, 2 or 3.

In another aspect of the invention, the invention comprises a crystal comprising a chimeric kinase receptor polypeptide, wherein said chimeric polypeptide comprises an amino acid sequence beginning at c-fms amino acid position 538 and continuing through c-fms amino acid position 922 wherein the native c-fms KID is replaced with a KID sequence comprising a heterologous KID amino acid sequence beginning at c-fms amino acid positions 672-688, or a fragment of the chimeric kinase receptor polypeptide, wherein said crystal comprises a crystal structure defined by one or more structure coordinates of c-fms amino acid residues Lys 586, Leu 588, Gly 589, Val 596, Glu 598, Ala 614, Lys 616, Val 647, Thr 663, Glu 664, Tyr 665, Cys 666, Cys 667, Tyr 668, Gly 669, Asp 670, Arg 677, Arg 782, Leu 785, Asp 796, Phe 797, Gly 798, Leu 799, Ala 800, Arg 801, Asp 802 according to Tables 1, 2 or 3- or similar structure coordinates for said amino acids comprising a root mean square deviation of non-hydrogen atoms of less than about 0.75 Å when superimposed on the non-hydrogen atom positions of the corresponding atomic coordinates of Tables 1, 2 or 3.

In another aspect of the invention, the invention comprises a crystal comprising a chimeric kinase receptor polypeptide, wherein said chimeric polypeptide comprises an amino acid sequence beginning at c-fms amino acid position 538 and continuing through c-fms amino acid position 922 wherein the native c-fms KID is replaced with a KID sequence comprising a heterologous KID amino acid sequence beginning at c-fms amino acid positions 672-688, or a fragment of the chimeric kinase receptor polypeptide, wherein said crystal comprises a crystal structure defined by structure coordinates of c-fms amino acid residues Lys 586, Leu 588, Gly 589, Val 596, Glu 598, Ala 614, Lys 616, Val 647, Thr 663, Glu 664, Tyr 665, Cys 666, Cys 667, Tyr 668, Gly 669, Asp 670, Arg 782, Asn 783, Leu 785, Asp 796, Phe 797, Leu 799, Ala 800, Arg 801 according to Tables 1, 2 or 3 or similar structure coordinates for said amino acids comprising a root mean square deviation of non-hydrogen atoms of less than about 1.5 Å when superimposed on the non-hydrogen atom positions of the corresponding atomic coordinates of Tables 1, 2 or 3.

In another aspect of the invention, the invention comprises a crystal comprising a chimeric kinase receptor polypeptide, wherein said chimeric polypeptide comprises an amino acid sequence beginning at c-fms amino acid position 538 and continuing through c-fms amino acid position 922 wherein the native c-fms KID is replaced with a KID sequence comprising a heterologous KID amino acid sequence beginning at c-fms amino acid positions 672-688, or a fragment of the chimeric kinase receptor polypeptide, wherein said crystal comprises a crystal structure defined by structure coordinates of c-fms amino acid residues Lys 586, Leu 588, Gly 589, Val 596, Glu 598, Ala 614, Lys 616, Val 647, Thr 663, Glu 664, Tyr 665, Cys 666, Cys 667, Tyr 668, Gly 669, Asp 670, Arg 782, Asn 783, Leu 785, Asp 796, Phe 797, Leu 799, Ala 800, Arg 801 according to Tables 1, 2 or 3 or similar structure coordinates for said amino acids comprising a root mean square deviation of non-hydrogen atoms of less than about 0.75 Å when superimposed on the non-hydrogen atom positions of the corresponding atomic coordinates of Tables 1, 2 or 3.

In yet a different aspect, any of the crystals further comprise a ligand, wherein the ligand is an ATP-binding pocket ligand. In one embodiment, the ATP-binding pocket ligand is a small molecule inhibitor.

In one embodiment, small molecule inhibitor is an arylamide compound or a derivative thereof. In another embodiment, the small molecule inhibitor is a quinolone compound or a derivative thereof. In a preferred embodiment, the arylamide compound is 5-cyano-furan-2-carboxylic acid [5-hydroxymethyl-2-(4-methyl-piperidine-1-yl)-phenyl]-amide or derivative thereof. In another preferred embodiment, the quinolone compound is 6-Chloro-3-(3-methyl-isoxazol-5-yl)-4-phenyl-1H-quinolin-2-one or a derivative thereof.

In one aspect of the invention, the crystal-ligand complex has a space group of R3. (Form I). In another aspect of the invention, the crystal-ligand complex has a space group of sg=P2₁2₁2₁. (Form II). In yet a different aspect, the crystal effectively diffracts X-rays for determination of atomic coordinates to a resolution of at least about 1.9 Å (Form I). In another aspect, the crystal effectively diffracts X-rays for determination of atomic coordinates to a resolution of at least about 3.0 Å (Form II).

Also included in the invention is a crystal comprising a unit cell having dimensions consisting of: a=81.07; b=81.07; c=144.67; alpha=90; beta=90; gamma=120. In one embodiment, the crystal comprises a unit cell having dimensions consisting of a=53.1; b=72.4; c=91.7; alpha=90; beta=90; gamma=90.

The invention also includes a crystal comprising a polypeptide which comprises a peptide having at least 95% amino acid sequence identity to SEQ ID NO. 2 (FMS/FGFR1 chimera); SEQ ID NO. 4 (FMS/tie chimera) or SEQ ID NO: 6 (FMS/irk chimera). In one embodiment, the crystal comprises a peptide having at least 95% sequence identity to SEQ ID NO. 2.

In one aspect, the crystal comprises SEQ ID NO: 2 comprising an atomic structure characterized by the coordinates of Tables 1, 2 or 3. In another aspect the invention includes an isolated nucleic acid molecule encoding any of the chimeric polypeptides or polypeptides disclosed above, a vector comprising the nucleic acid, a host cell comprising the vector and a method of producing the polypeptide by culturing the host cell.

Also included in the invention is a computer system comprising (a) a database containing information on the three dimensional structure of a crystal comprising a c-fms chimera, or a fragment or a target structural motif or derivative thereof, and a ligand, wherein the ligand is a small molecule inhibitor, stored on a computer readable storage medium; and, (b) a user interface to view the information.

In one aspect of the invention, the information comprises diffraction data obtained from a crystal comprising SEQ ID NO: 2, 4 or 6. In another aspect, the information comprises an electron density map of a crystal form comprising SEQ ID NO: 2, 4 or 6. In yet a different aspect, the information comprises the structure coordinates of Tables 1, 2 or 3 or homologous structure coordinates for the amino acids of SEQ ID NO: 2 comprising a root mean square deviation of non-hydrogen atoms of less than about 1.5 Å when superimposed on the non-hydrogen atom positions of the corresponding atomic coordinates of Tables 1, 2 or 3. In one embodiment, the information comprises structure coordinates for amino acid residues of SEQ ID NO: 2 comprising a root mean square deviation of non-hydrogen atoms of less than about 0.75 Å when superimposed on the non-hydrogen atom positions of the corresponding atomic coordinates of Tables 1, 2 or 3.

In yet a different embodiment, the information comprises the structure coordinates for one or more amino acid residues Trp 550, Lys 586, Thr 587, Leu 588, Gly 589, Val 596, Glu 598, Ala 614, Val 615, Lys 616, Glu 633, Met 637, Leu 640, Ile 646, Val 647, Val 661, Thr 663, Glu 664, Tyr 665, Cys 666, Cys 667, Tyr 668, Gly 669, Asp 670, Asn 673, Arg 677, Cys 774, Ile 775, His 776, Arg 782, Asn 783, Leu 785, Ile 794, Gly 795, Asp 796, Phe 797, Gly 798, Leu 799, Ala 800, Arg 801, Asp 802 according to Tables 1, 2 or 3 or similar structure coordinates for said amino acids comprising a root mean square deviation of non-hydrogen atoms of less than about 1.5 Å when superimposed on the non-hydrogen atom positions of the corresponding atomic coordinates of Tables 1, 2 or 3. In another aspect, the information further comprises the structure coordinates for one or more amino acid residues Trp 550, Lys 586, Thr 587, Leu 588, Gly 589, Val 596, Glu 598, Ala 614, Val 615, Lys 616, Glu 633, Met 637, Leu 640, Ile 646, Val 647, Val 661, Thr 663, Glu 664, Tyr 665, Cys 666, Cys 667, Tyr 668, Gly 669, Asp 670, Asn 673, Arg 677, Cys 774, Ile 775, His 776, Arg 782, Asn 783, Leu 785, Ile 794, Gly 795, Asp 796, Phe 797, Gly 798, Leu 799, Ala 800, Arg 801, Asp 802 according to Tables 1, 2 or 3 or similar structure coordinates for said amino acids comprising a root mean square deviation of non-hydrogen atoms of less than about 0.75 Å when superimposed on the non-hydrogen atom, positions of the corresponding atomic coordinates of Tables 1, 2 or 3.

In a different aspect, the computer system comprises a crystal structure defined by structure coordinates of one or more c-fms amino acid residues Lys 586, Leu 588, Gly 589, Val 596, Glu 598, Ala 614, Lys 616, Val 647, Thr 663, Glu 664, Tyr 665, Cys 666, Cys 667, Tyr 668, Gly 669, Asp 670, Arg 677, Arg 782, Leu 785, Asp 796, Phe 797, Gly 798, Leu 799, Ala 800, Arg 801, Asp 802 according to Tables 1, 2 or 3 or similar structure coordinates for said amino acids comprising a root mean square deviation of non-hydrogen atoms of less than about 1.5 Å when superimposed on the non-hydrogen atom positions of the corresponding atomic coordinates of Tables 1, 2 or 3. In another aspect, the computer system comprises a crystal structure defined by structure coordinates of one or more c-fms amino acid residues Lys 586, Leu 588, Gly 589, Val 596, Glu 598, Ala 614, Lys 616, Val 647, Thr 663, Glu 664, Tyr 665, Cys 666, Cys 667, Tyr 668, Gly 669, Asp 670, Arg 677, Arg 782, Leu 785, Asp 796, Phe 797, Gly 798, Leu 799, Ala 800, Arg 801, Asp 802 according to Tables 1, 2 or 3 or similar structure coordinates for said amino acids comprising a root mean square deviation of non-hydrogen atoms of less than about 0.75 Å when superimposed on the non-hydrogen atom positions of the corresponding atomic coordinates of Tables 1, 2 or 3.

In yet a different aspect, the computer system comprises a crystal structure defined by structure coordinates of c-fms amino acid residues Lys 586, Leu 588, Gly 589, Val 596, Glu 598, Ala 614, Lys 616, Val 647, Thr 663, Glu 664, Tyr 665, Cys 666, Cys 667, Tyr 668, Gly 669, Asp 670, Arg 782, Asn 783, Leu 785, Asp 796, Phe 797, Leu 799, Ala 800, Arg 801 according to Tables 1, 2 or 3 or similar structure coordinates for said amino acids comprising a root mean square deviation of non-hydrogen atoms of less than about 1.5 Å when superimposed on the non-hydrogen atom positions of the corresponding atomic coordinates of Tables 1, 2 or 3. In one embodiment, the computer system comprises a crystal structure defined by structure coordinates of c-fms amino acid residues Lys 586, Leu 588, Gly 589, Val 596, Glu 598, Ala 614, Lys 616, Val 647, Thr 663, Glu 664, Tyr 665, Cys 666, Cys 667, Tyr 668, Gly 669, Asp 670, Arg 782, Asn 783, Leu 785, Asp 796, Phe 797, Leu 799, Ala 800, Arg 801 according to Tables 1, 2 or 3 or similar structure coordinates for said amino acids comprising a root mean square deviation of non-hydrogen atoms of less than about 0.75 Å when superimposed on the non-hydrogen atom positions of the corresponding atomic coordinates of Tables 1, 2 or 3.

The invention further includes a method of evaluating the potential of an agent to associate with c-fms chimeric polypeptides comprising (a) exposing the c-fms chimera to the agent; and (b) detecting the association of the agent to one or more c-fms amino acid residues selected from the group consisting of (i) Trp 550, Lys 586, Thr 587, Leu 588, Gly 589, Val 596, Glu 598, Ala 614, Val 615, Lys 616, Glu 633, Met 637, Leu 640, Ile 646, Val 647, Val 661, Thr 663, Glu 664, Tyr 665, Cys 666, Cys 667, Tyr 668, Gly 669, Asp 670, Asn 673, Arg 677, Cys 774, Ile 775, His 776, Arg 782, Asn 783, Leu 785, Ile 794, Gly 795, Asp 796, Phe 797, Gly 798, Leu 799, Ala 800, Arg 801, Asp 802; (ii) Lys 586, Leu 588, Gly 589, Val 596, Glu 598, Ala 614, Lys 616, Val 647, Thr 663, Glu 664, Tyr 665, Cys 666, Cys 667, Tyr 668, Gly 669, Asp 670, Arg 677, Arg 782, Leu 785, Asp 796, Phe 797, Gly 798, Leu 799, Ala 800, Arg 801, Asp 802; and, (iii) Lys 586, Leu 588, Gly 589, Val 596, Glu 598, Ala 614, Lys 616, Val 647, Thr 663, Glu 664, Tyr 665, Cys 666, Cys 667, Tyr 668, Gly 669, Asp 670, Arg 782, Asn 783, Leu 785, Asp 796, Phe 797, Leu 799, Ala 800, Arg 801 thereby evaluating the potential. The agent can be a virtual compound.

In addition, also included is a method of evaluating the potential of an agent to associate with the polypeptide, comprising: (a) exposing the polypeptide to the agent; and (b) detecting the level of association of the agent to the polypeptide, thereby evaluating the potential of the agent to associate with the polypeptide; the agent can be a virtual compound. In one aspect, step (a) comprises comparing the atomic structure of the compound to the three dimensional structure of a c-fms chimeric polypeptide. In one embodiment, the comparing comprises employing a computational means to perform a fitting operation between the compound and at least one binding site of a c-fms chimera.

In one embodiment, the binding site is defined by one or more structure coordinates for amino acids Trp 550, Lys 586, Thr 587, Leu 588, Gly 589, Val 596, Glu 598, Ala 614, Val 615, Lys 616, Glu 633, Met 637, Leu 640, Ile 646, Val 647, Val 661, Thr 663, Glu 664, Tyr 665, Cys 666, Cys 667, Tyr 668, Gly 669, Asp 670, Asn 673, Arg 677, Cys 774, Ile 775, His 776, Arg 782, Asn 783, Leu 785, Ile 794, Gly 795, Asp 796, Phe 797, Gly 798, Leu 799, Ala 800, Arg 801, Asp 802 according to Tables 1, 2 or 3 or similar structure coordinates for said amino acids of a c-fms chimera comprising a root mean square deviation of non-hydrogen atoms of less than about 1.5 Å when superimposed on the non-hydrogen atom positions of the corresponding atomic coordinates of Tables 1, 2 or 3. In another aspect, the binding site is defined by one or more structure coordinates for amino acids Trp 550, Lys 586, Thr 587, Leu 588, Gly 589, Val 596, Glu 598, Ala 614, Val 615, Lys 616, Glu 633, Met 637, Leu 640, Ile 646, Val 647, Val 661, Thr 663, Glu 664, Tyr 665, Cys 666, Cys 667, Tyr 668, Gly 669, Asp 670, Asn 673, Arg 677, Cys 774, Ile 775, His 776, Arg 782, Asn 783, Leu 785, Ile 794, Gly 795, Asp 796, Phe 797, Gly 798, Leu 799, Ala 800, Arg 801, Asp 802 according to Tables 1, 2 or 3 or similar structure coordinates for said amino acids of a c-fms chimera comprising a root mean square deviation of non-hydrogen atoms of less than about 0.75 Å when superimposed on the non-hydrogen atom positions of the corresponding atomic coordinates of Tables 1, 2 or 3.

In one embodiment, the method of comprises a crystal structure defined by one or more structure coordinates of c-fms amino acid residues Lys 586, Leu 588, Gly 589, Val 596, Glu 598, Ala 614, Lys 616, Val 647 Thr 663, Glu 664, Tyr 665, Cys 666, Cys 667, Tyr 668, Gly 669, Asp 670, Arg 677, Arg 782, Leu 785, Asp 796, Phe 797, Gly 798, Leu 799, Ala 800, Arg 801, Asp 802 according to Tables 1, 2 or 3 or similar structure coordinates for said amino acids comprising a root mean square deviation of non-hydrogen atoms of less than about 1.5 Å when superimposed on the non-hydrogen atom positions of the corresponding atomic coordinates of Tables 1, 2 or 3. In a different embodiment, the method comprises a crystal structure defined by one or more structure coordinates of c-fms amino acid residues Lys 586, Leu 588, Gly 589, Val 596, Glu 598, Ala 614, Lys 616, Val 647, Thr 663, Glu 664, Tyr 665, Cys 666, Cys 667, Tyr 668, Gly 669, Asp 670, Arg 677, Arg 782, Leu 785, Asp 796, Phe 797, Gly 798, Leu 799, Ala 800, Arg 801, Asp 802 according to Tables 1, 2 or 3 or similar structure coordinates for said amino acids comprising a root mean square deviation of non-hydrogen atoms of less than about 0.75 Å when superimposed on the non-hydrogen atom positions of the corresponding atomic coordinates of Tables 1, 2 or 3.

In yet a different embodiment, the method comprises a crystal structure defined by structure coordinates of c-fms amino acid residues Lys 586, Leu 588, Gly 589, Val 596, Glu 598, Ala 614, Lys 616, Val 647, Thr 663, Glu 664, Tyr 665, Cys 666, Cys 667, Tyr 668, Gly 669, Asp 670, Arg 782, Asn 783, Leu 785, Asp 796, Phe 797, Leu 799, Ala 800, Arg 801 according to Tables 1, 2 or 3 or similar structure coordinates for said amino acids comprising a root mean square deviation of non-hydrogen atoms of less than about 1.5 Å when superimposed on the non-hydrogen atom positions of the corresponding atomic coordinates of Tables 1, 2 or 3. In another embodiment, the method comprises a crystal structure defined by structure coordinates of c-fms amino acid residues Lys 586, Leu 588, Gly 589, Val 596, Glu 598, Ala 614, Lys 616, Val 647, Thr 663, Glu 664, Tyr 665, Cys 666, Cys 667, Tyr 668, Gly 669, Asp 670, Arg 782, Asn 783, Leu 785, Asp 796, Phe 797, Leu 799, Ala 800, Arg 801 according to Tables 1, 2 or 3 or similar structure coordinates for said amino acids comprising a root mean square deviation of non-hydrogen atoms of less than about 0.75 Å when superimposed on the non-hydrogen atom positions of the corresponding atomic coordinates of Tables 1, 2 or 3. In a different aspect of the method, the agent is exposed to a crystalline c-fms chimera and the detecting of step (b) comprises determining the three dimensional structure of the agent-c-fms chimera complex.

In a different aspect, the invention includes a method of identifying a potential agonist or antagonist against a c-fms chimera comprising employing the three dimensional structure of the c-fms chimera cocrystallized with a small molecule inhibitor to design or select a potential agonist or antagonist. In one embodiment, the three dimensional structure corresponds to the atomic structure characterized by the coordinates of Tables 1, 2 or 3 or similar structure coordinates for said c-fms chimera comprising a root mean square deviation of non-hydrogen atoms of less than about 1.5 Å when superimposed on the non-hydrogen atom positions of the corresponding atomic coordinates of Tables 1, 2 or 3. In another embodiment, the method further comprises the steps of: (b) synthesizing the potential agonist or antagonist; and (c) contacting the potential agonist or antagonist with a chimeric c-fms polypeptide.

The invention is also directed to a method of locating the attachment site of an inhibitor to a c-fms chimeric polypeptide, comprising (a) obtaining X-ray diffraction data for a crystal of a chimeric c-fms polypeptide; (b) obtaining X-ray diffraction data for a complex of a chimeric c-fms polypeptide and the inhibitor; (c) subtracting the X-ray diffraction data obtained in step (a) from the X-ray diffraction data obtained in step (b) to obtain the difference in the X-ray diffraction data; (d) obtaining phases that correspond to X-ray diffraction data obtained in step (a); (e) utilizing the phases obtained in step (d) and the difference in the X-ray diffraction data obtained in step (c) to compute a difference Fourier image of the inhibitor; and (f) locating the attachment site of the inhibitor based on the computations obtained in step (e).

In a different aspect, the invention is directed to a method of obtaining a modified inhibitor comprising (a) obtaining a crystal comprising a chimeric c-fms polypeptide and an inhibitor; (b) obtaining the atomic coordinates of the crystal; (c) using the atomic coordinates and one or more molecular modeling techniques to determine how to modify the interaction of the inhibitor with the chimeric c-fms polypeptide; and (d) modifying the inhibitor based on the determinations obtained in step (c) to produce a modified inhibitor.

In one embodiment, the crystal comprises a peptide selected from the group consisting of: a peptide having SEQ ID NO: 2; a peptide having SEQ ID NO: 4 and a peptide having SEQ ID NO: 6. In another embodiment, the one or more molecular modeling techniques are selected from the group consisting of graphic molecular modeling and computational chemistry. In one embodiment, step (b) comprises detecting the interaction of the inhibitor to one or more amino acid residues selected from the group consisting of (i) Trp 550, Lys 586, Thr 587, Leu 588, Gly 589, Val 596, Glu 598, Ala 614, Val 615, Lys 616, Glu 633, Met 637, Leu 640, Ile 646, Val 647, Val 661, Thr 663, Glu 664, Tyr 665, Cys 666, Cys 667, Tyr 668, Gly 669, Asp 670, Asn 673, Arg 677, Cys 774, Ile 775, His 776, Arg 782, Asn 783, Leu 785, Ile 794, Gly 795, Asp 796, Phe 797, Gly 798, Leu 799, Ala 800, Arg 801, Asp 802; (ii) Lys 586, Leu 588, Gly 589, Val 596, Glu 598, Ala 614, Lys 616, Val 647, Thr 663, Glu 664, Tyr 665, Cys 666, Cys 667, Tyr 668, Gly 669, Asp 670, Arg 677, Arg 782, Leu 785, Asp 796, Phe 797, Gly 798, Leu 799, Ala 800, Arg 801, Asp 802; and, (iii) Lys 586, Leu 588, Gly 589, Val 596, Glu 598, Ala 614, Lys 616, Val 647, Thr 663, Glu 664, Tyr 665, Cys 666, Cys 667, Tyr 668, Gly 669, Asp 670, Arg 782, Asn 783, Leu 785, Asp 796, Phe 797, Leu 799, Ala 800, Arg 801. In one embodiment of the method, an inhibitor of a chimeric c-fms polypeptide is identified.

The invention further includes an isolated protein fragment comprising a binding pocket or active site defined by one or more structure coordinates of chimeric c-fms amino acid residues selected from the group consisting of (i) Trp 550, Lys 586, Thr 587, Leu 588, Gly 589, Val 596, Glu 598, Ala 614, Val 615, Lys 616, Glu 633, Met 637, Leu 640, Ile 646, Val 647, Val 661, Thr 663, Glu 664, Tyr 665, Cys 666, Cys 667, Tyr 668, Gly 669, Asp 670, Asn 673, Arg 677, Cys 774, Ile 775, His 776, Arg 782, Asn 783, Leu 785, Ile 794, Gly 795, Asp 796, Phe 797, Gly 798, Leu 799, Ala 800, Arg 801, Asp 802; (ii) Lys 586, Leu 588, Gly 589, Val 596, Glu 598, Ala 614, Lys 616, Val 647, Thr 663, Glu 664, Tyr 665, Cys 666, Cys 667, Tyr 668, Gly 669, Asp 670, Arg 677, Arg 782, Leu 785, Asp 796, Phe 797, Gly 798, Leu 799, Ala 800, Arg 801, Asp 802; and, (iii) Lys 586, Leu 588, Gly 589, Val 596, Glu 598, Ala 614, Lys 616, Val 647, Thr 663, Glu 664, Tyr 665, Cys 666, Cys 667, Tyr 668, Gly 669, Asp 670, Arg 782, Asn 783, Leu 785, Asp 796, Phe 797, Leu 799, Ala 800, Arg 801. The invention also includes a fragment linked to a solid support, an isolated nucleic acid molecule encoding the fragment, a vector comprising the nucleic acid molecule, a host cell comprising the vector, and, a method of producing a protein fragment comprising culturing the host cell under conditions in which the fragment is expressed.

The invention also includes a method of screening for an agent that associates with a chimeric c-fms polypeptide, comprising (a) exposing a protein molecule fragment to the agent; and (b) detecting the level of association of the agent to the fragment. Also included in the invention is a kit comprising the protein molecule fragment.

In another aspect of the invention, the invention is directed to a method for the production of a crystal complex comprising a chimeric c-fms chimeric polypeptide-ligand comprising (a) contacting the chimeric c-fms polypeptide with the ligand in a suitable solution and, (b) crystallizing the resulting complex of chimeric c-fms polypeptide-ligand from the solution. In one embodiment, the invention includes a method for the production of a crystal comprising crystallizing a peptide comprising a sequence selected from the group consisting of SEQ ID NO: 2, 4 or 6 with a potential inhibitor. In another embodiment, the method further comprises contacting the crystalline chimeric c-fms polypeptide-ligand complex with another ligand in a suitable solution to replace the bound ligand.

The invention also includes methods or identifying a potential inhibitor of a chimeric c-fms polypeptide comprising (a) using a three dimensional structure of a chimeric c-fms polypeptide as defined by atomic coordinates according to Tables 1, 2 or 3 or similar structure coordinates for the amino acids of a c-fms chimera comprising a root mean square deviation of non-hydrogen atoms of less than about 1.5 Å when superimposed on the non-hydrogen atom positions of the corresponding atomic coordinates of Tables 1, 2 or 3; (b) replacing one or more chimeric c-fms polypeptide amino acids selected from the group consisting of (i) Trp 550, Lys 586, Thr 587, Leu 588, Gly 589, Val 596, Glu 598, Ala 614, Val 615, Lys 616, Glu 633, Met 637, Leu 640, Ile 646, Val 647, Val 661, Thr 663, Glu 664, Tyr 665, Cys 666, Cys 667, Tyr 668, Gly 669, Asp 670, Asn 673, Arg 677, Cys 774, Ile 775, His 776, Arg 782, Asn 783, Leu 785, Ile 794, Gly 795, Asp 796, Phe 797, Gly 798, Leu 799, Ala 800, Arg 801, Asp 802; (ii) Lys 586, Leu 588, Gly 589, Val 596, Glu 598, Ala 614, Lys 616, Val 647, Thr 663, Glu 664, Tyr 665, Cys 666, Cys 667, Tyr 668, Gly 669, Asp 670, Arg 677, Arg 782, Leu 785, Asp 796, Phe 797, Gly 798, Leu 799, Ala 800, Arg 801, Asp 802; and, (iii) Lys 586, Leu 588, Gly 589, Val 596, Glu 598, Ala 614, Lys 616, Val 647, Thr 663, Glu 664, Tyr 665, Cys 666, Cys 667, Tyr 668, Gly 669, Asp 670, Arg 782, Asn 783, Leu 785, Asp 796, Phe 797, Leu 799, Ala 800, Arg 801 in the three-dimensional structure with a different amino acid to produce a modified three-dimensional structure; and, (c) using the modified three-dimensional structure to design or select the potential inhibitor. In one aspect, the method further comprises d) synthesizing said potential inhibitor. In a different aspect, the method further comprises e) contacting said potential inhibitor with said modified chimeric c-fms polypeptide in the presence of a ligand to test the ability of said potential inhibitor to inhibit a chimeric c-fms polypeptide or said modified chimeric c-fms polypeptide; and the inhibitor identified. In one embodiment, the replacing of one or more amino acid residues further comprises replacing SEQ ID NO: 2 amino acid residues selected from the group consisting of Lys 586, Leu 588, Gly 589, Val 596, Glu 598, Ala 614, Lys 616, Val 647, Thr 663, Glu 664, Tyr 665, Cys 666, Cys 667, Tyr 668, Gly 669, Asp 670, Arg 782, Asn 783, Leu 785, Asp 796, Phe 797, Leu 799, Ala 800, Arg 801. In yet a different aspect, the potential inhibitor is selected from a database. In another aspect, the potential inhibitor is designed de novo. In one embodiment, the potential inhibitor is designed from a known inhibitor. In yet a different embodiment, the step of employing said modified three-dimensional structure to design or select said potential inhibitor comprises the steps of: (a) identifying chemical entities or fragments capable of associating with a modified chimeric c-fms polypeptide; and (b) assembling the identified chemical entities or fragments into a single molecule to provide the structure of said potential inhibitor. In one aspect, the potential inhibitor is a competitive inhibitor. In a different aspect, the potential inhibitor is a non-competitive or uncompetitive inhibitor. In one embodiment, the potential inhibitor is an irreversible inhibitor.

The present invention includes methods of producing and using three-dimensional structure information derived from c-fms and c-fms chimeric polypeptides and inhibitory compounds which form a complex with c-fms and c-fms chimeric polypeptides and prevent c-fms and c-fms chimeras from interacting with their naturally occurring ligand or ligands. The present invention also includes specific crystallization to obtain crystals of the c-fms-ligand (inhibitor) complex. The crystals are subsequently used to obtain a 3-dimensional structure of the complex using X-ray crystallography (or NMR) and the obtained data is used for rational drug discovery design with the aim to improve the complex formation between c-fms and its chimeras and the inhibitor, and, also to improve the inhibition of the binding of c-fms ligands. In this invention the KID was replaced by the shorter KID derived from the FGFR1 receptor [24]. Additional constructs also include c-fms chimeras derived from replacing the native KID with the KID of tie2 or IRK.

### BRIEF DESCRIPTION OF THE DRAWINGS

FIGS. 1 A and B. Ribbons representation of the overall fold of c-fms complexed with arylamide 1183648 (A) or quinolone 793693 (B) compounds. The secondary structure elements are coded in magenta (α-helices) or yellow (β-sheets). The positions of the termini are indicated by N and C, respectively. Important structural elements are color-coded: nucleotide binding loop (blue), activation loop (red), catalytic loop (green), hinge region (salmon) and kinase insert domain (KID) (cyan). Disordered regions are approximated by dotted lines. Figure created in PyMol [25].

FIGS. 2A and B. Combined ribbons and ball-and-stick representation of the c-fms binding pocket complexed with arylamide 1183648 (A) or quinolone 793693 (B) compounds. Color coding is the same as in FIG. 1. The ribbons representation for the hinge region was omitted and replaced with a complete ball-and-stick representation of the relevant amino acids. Figure created in PyMol [25].

FIG. 3. Nucleotide sequence of the cfms-FGFR1 chimera beginning at Tyr 538 of c-fms (SEQ ID NO: 1).

FIG. 4. Amino acid sequence of the c-FMS-FGFR1 chimera (SEQ ID NO: 2).

FIG. 5. Nucleotide sequence of the cfms-TIE2 chimera beginning at Tyr 538 of c-fms (SEQ ID NO: 3).

FIG. 6. Amino acid sequence of the c-FMS-TIE2 chimera (SEQ ID NO: 4).

FIG. 7. Nucleotide sequence of the c-FMs-irk chimera (SEQ ID NO: 5)

FIG. 8. Amino acid sequence of the c-FMS-irk chimera (SEQ ID NO: 6)

FIG. 9. Nucleotide sequence of wild-type c-fms (SEQ ID NO: 7)

FIG. 10. Amino acid sequence of wild-type c-fms (SEQ ID NO: 8)

FIG. 11. FIG. 11 describes the construct design process exemplified for the c-fms-FGFR1-chimera.

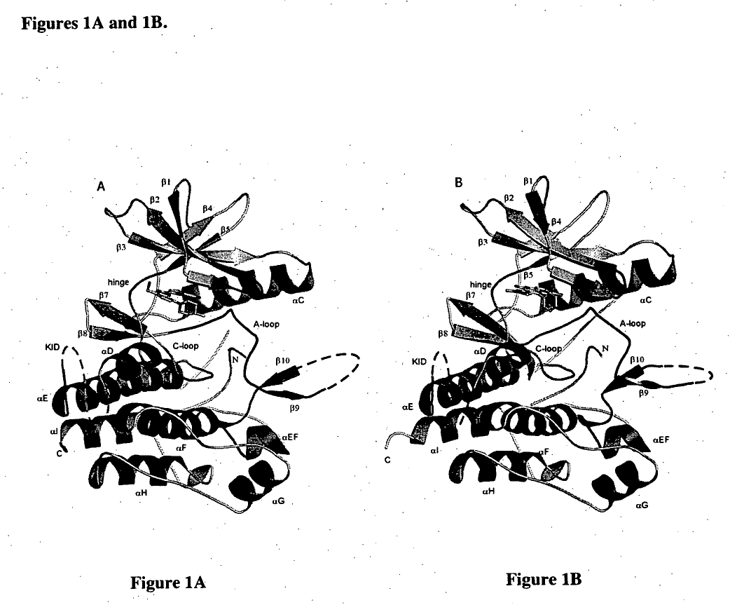

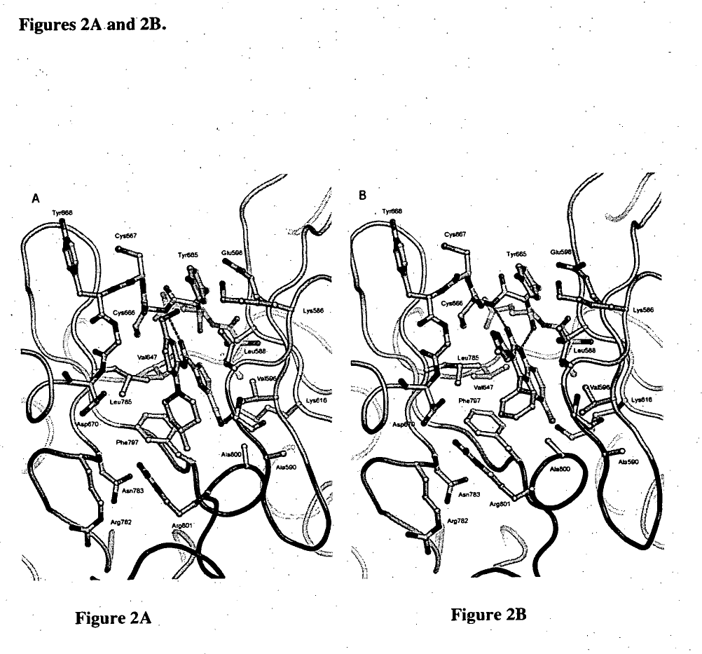

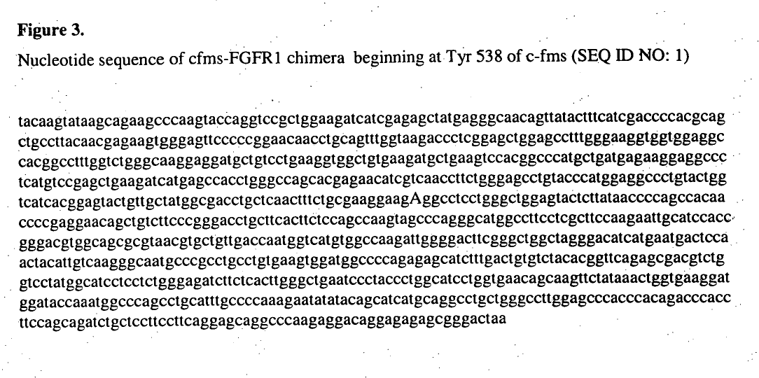

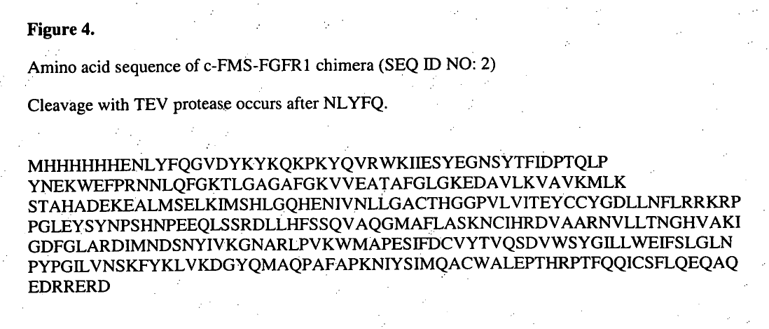

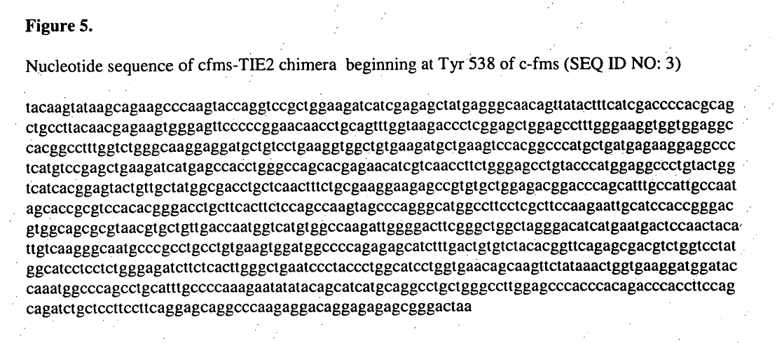

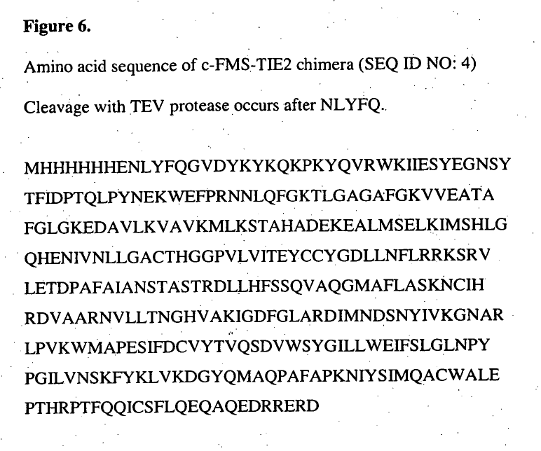

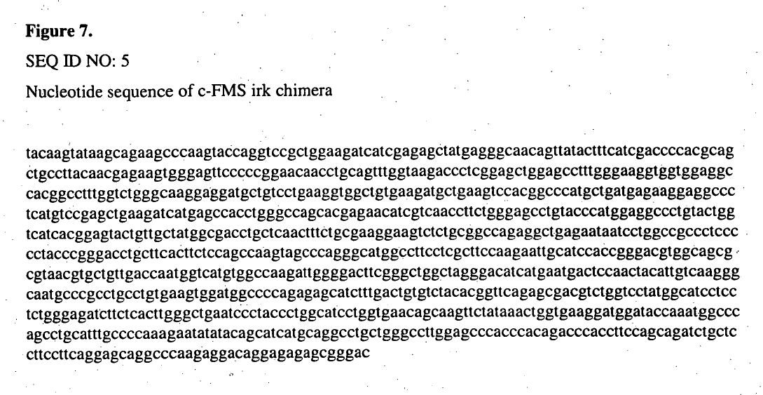

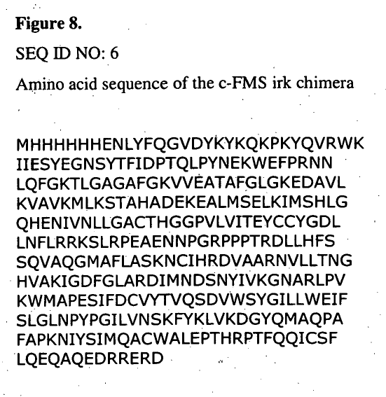

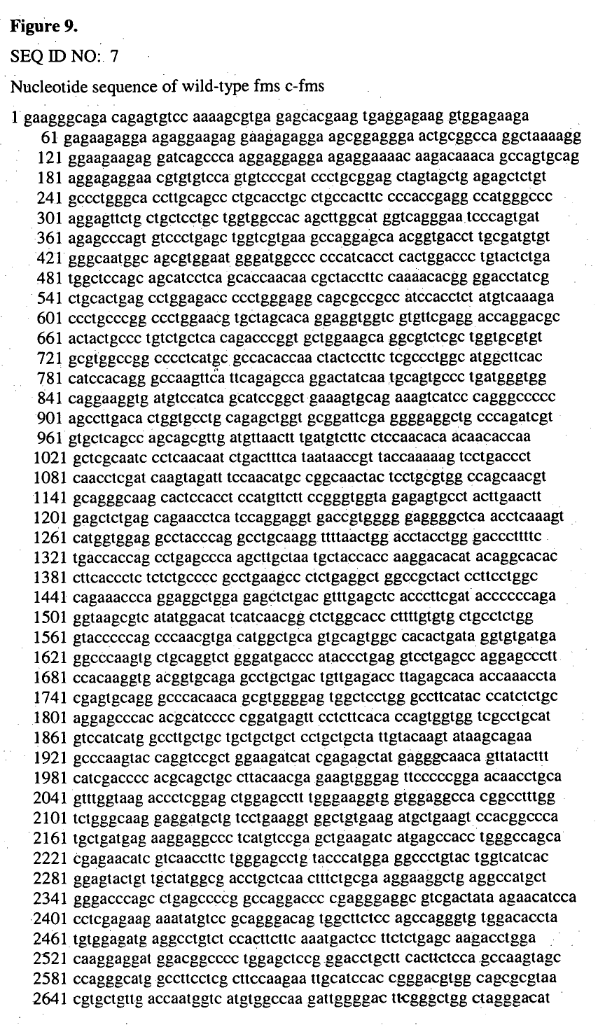

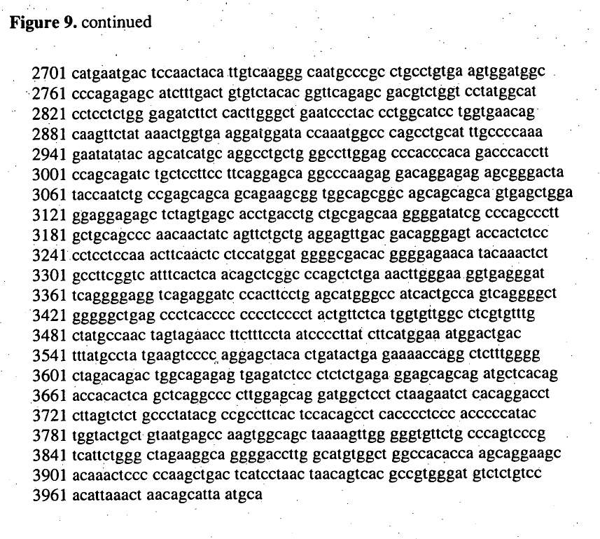

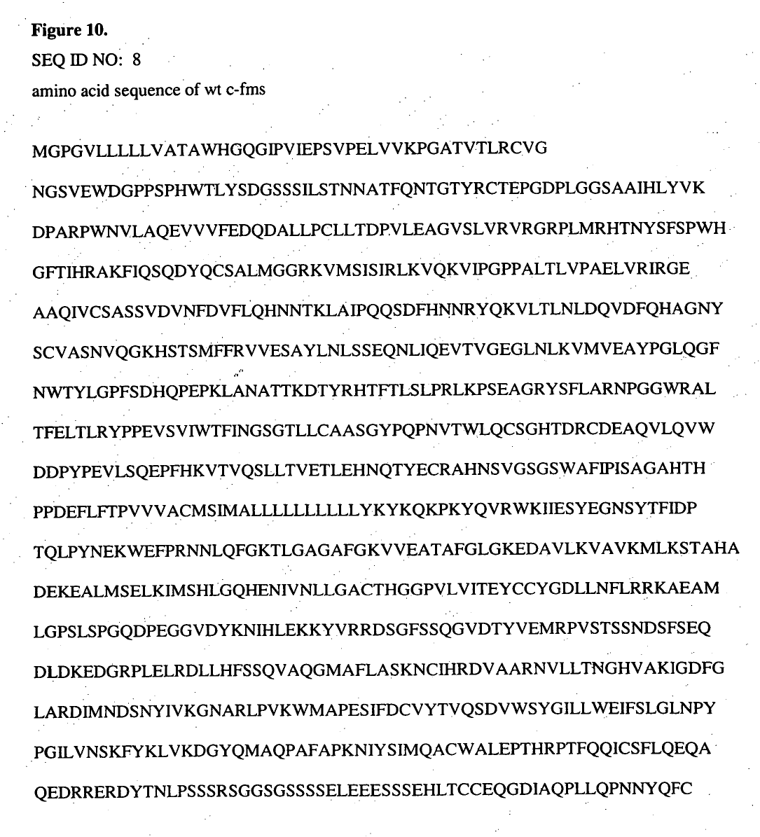

### DETAILED DESCRIPTION OF THE INVENTION

Definitions

As is generally the case in biotechnology and chemistry, the description of the present invention has required the use of a number of terms of art. Although it is not practical to do so exhaustively, definitions for some of these terms are provided here for ease of reference. Unless defined otherwise, all technical and scientific terms used herein have the same meaning as commonly understood by one of ordinary skill in the art to which this invention belongs. Definitions for other terms also appear elsewhere herein. However, the definitions provided here and elsewhere herein should always be considered in determining the intended scope and meaning of the defined terms. Although any methods and materials similar or equivalent to those described herein can be used in the practice of the present invention, the preferred methods and materials are described.

As used herein, the term “atomic coordinates” or “structure coordinates” refers to mathematical coordinates that describe the positions of atoms in crystals of c-fms chimeras in Protein Data Bank (PDB) format, including X, Y, Z and B, for each atom. The diffraction data obtained from the crystals are used to calculate an electron density map of the repeating unit of the crystal. The electron density maps may be used to establish the positions (i.e., coordinates X, Y and Z) of the individual atoms within the crystal. Those of skill in the art understand that a set of structure coordinates determined by X-ray crystallography is not without standard error. For the purpose of this invention, any set of structure coordinates for c-fms chimeras from any source having a root mean square deviation of non-hydrogen atoms of less than about 1.5 Å when superimposed on the non-hydrogen atom positions of the corresponding atomic coordinates of Tables 1, 2 or 3 are considered substantially identical or homologous. In a preferred embodiment, any set of structure coordinates for c-fms chimeras from any source having a root mean square deviation of non-hydrogen atoms of less than about 0.75 Å when superimposed on the non-hydrogen atom positions of the corresponding atomic coordinates of Tables 1, 2 or 3 are considered substantially identical or homologous.

As used herein, the term “unit cell” means the fundamental portion of a crystal structure that is repeated infinitely by translation in three dimensions. A unit cell is characterized by three vectors a, b, and c, not located in one plane, which form the edges of a parallelepiped. Angles alpha, beta and gamma define the angles between the vectors: angle alpha is the angle between vectors b and c; angle beta is the angle between vectors a and c; and angle gamma is the angle between vectors a and b. The entire volume of a crystal can be constructed by regular assembly of unit cells. Each unit cell comprises a complete representation of the unit of pattern, the repetition of which builds up the crystal. See, for example, U.S. Appl. No. 2004/0002145.

As used herein, the term “asymmetric unit” (ASU) means part of a symmetric object, which by itself does not posses any symmetry and from which the whole unit cell is built up by the application of symmetry operations of its point group. See, for example, U.S. Appl. No. 2004/0002145.

As used herein, the term “space group” means a group or array of operations consistent with an infinitely extended regularly repeating pattern. It is the symmetry of a three-dimensional structure, or the arrangement of symmetry elements of a crystal. There are 230 space group symmetries possible; however, there are only 65 space group symmetries available for biological structures. See, for example, U.S. Appl. No. 2004/0002145.

The term “atom type” refers to the chemical element whose coordinates are measured. For instance, the first letter in a column in Table 1 identifies the element.

The terms “X,” “Y” and “Z” refer to the crystallographically-defined atomic position of the element measured with respect to the chosen crystallographic origin. The term “B” refers to a thermal factor that measures the mean variation of an atom's position with respect to its average position.

As used herein, the term “crystal” refers to any three-dimensional ordered array of molecules that diffracts X-rays.

As used herein, the term “carrier” in a composition refers to a diluent, adjuvant, excipient, or vehicle with which the product is mixed.

As used herein, the term “composition” refers to the combining of distinct elements or ingredients to form a whole. A composition comprises more than one element or ingredient. For the purposes of this invention, a composition will often, but not always, comprise a carrier.

As used herein, the term “SAR,” an abbreviation for Structure-Activity Relationships, collectively refers to the structure-activity/structure property relationships pertaining to the relationship(s) between a compound's activity/properties and its chemical structure.

As used herein, the term “molecular structure” refers to the three dimensional arrangement of molecules of a particular compound or complex of molecules (e.g., the three dimensional structure of a c-fms chimera and ligands that interact with the c-fms chimera).

As used herein, the term “molecular modeling” refers to the use of computational methods, preferably computer assisted methods, to draw realistic models of what molecules look like and to make predictions about structure activity relationships of ligands. The methods used in molecular modeling range from molecular graphics to computational chemistry.

As used herein, the term “molecular model” refers to the three dimensional arrangement of the atoms of a molecule connected by covalent bonds or the three dimensional arrangement of the atoms of a complex comprising more than one molecule, e.g., a protein-ligand complex.

As used herein, the term “molecular graphics” refers to 3D representations of the molecules, for instance, a 3D representation produced using computer assisted computational methods.

As used herein, the term “computational chemistry” refers to calculations of the physical and chemical properties of the molecules.

As used herein, the term “molecular replacement” refers to a method that involves generating a preliminary model of a crystal of whose coordinates are unknown, by orienting and positioning the said atomic coordinates described in the present invention so as best to account for the observed diffraction pattern of the unknown crystal. Phases can then be calculated from this model and combined with the observed amplitudes to give an approximate Fourier synthesis of the structure whose coordinates are unknown. (Rossmann, M. G., ed., “The Molecular Replacement Method,” Gordon & Breach, New York, 1972).

As used herein, the term “homolog” refers to the protein molecule or the nucleic acid molecule which encodes the protein, or a functional domain from said protein from a first source having at least about 30%, 40% or 50% sequence identity, or at least about 60%, 70% or 75% sequence identity, or at least about 80% sequence identity, or more preferably at least about 85% sequence identity, or even more preferably at least about 90% sequence identity, and most preferably at least about 95%, 97% or 99% amino acid or nucleotide sequence identity, with the protein, encoding nucleic acid molecule or any functional domain thereof, from a second source. The second source may be a version of the molecule from the first source that has been genetically altered by any available means to change the primary amino acid or nucleotide sequence or may be from the same or a different species than that of the first source.

As used herein, the term “active site” refers to regions on a protein or a structural motif of a protein that are directly involved in the function or activity of the c-fms chimera or c-fms protein.

As used herein, the terms “binding site” or “binding pocket” refer to a region of a protein or a molecular complex comprising the protein or polypeptide that, as a result of the primary amino acid sequence of the protein and/or its three-dimensional shape, favorably associates with another chemical entity or compound including ligands or inhibitors.

For the purpose of this invention, any active site, binding site or binding pocket defined by a set of structure coordinates for a protein or for a homolog of a protein from any source having a root mean square deviation of non-hydrogen atoms of less than about 1.5 Å when superimposed on the non-hydrogen atom positions of the corresponding atomic coordinates of Tables 1, 2 or 3 are considered substantially identical or homologous. In a more preferred embodiment, any set of structure coordinates for a protein or a homolog of a protein from any source having a root mean square deviation of non-hydrogen atoms of less than about 0.75 Å when superimposed on the non-hydrogen atom positions of the corresponding atomic coordinates of Tables 1, 2 or 3 are considered substantially identical or homologous.

The term “root mean square deviation” means the square root of the arithmetic mean of the squares of the deviations from the mean.

As used herein, the term “amino acids” refers to the L-isomers of the naturally occurring amino acids. The naturally occurring amino acids are glycine, alanine, valine, leucine, isoleucine, serine, methionine, threonine, phenylalanine, tyrosine, tryptophan, cysteine, proline, histidine, aspartic acid, asparagine, glutamic acid, glutamine, γ-carboxylglutamic acid, arginine, ornithine, and lysine. Unless specifically indicated, all amino acids are referred to in this application are in the L-form.

As used herein, the term “nonnatural amino acids” refers to amino acids that are not naturally found in proteins. For example, selenomethionine.

As used herein, the term “positively charged amino acid” includes any amino acids having a positively charged side chain under normal physiological conditions. Examples of positively charged naturally occurring amino acids are arginine, lysine, and histidine.

As used herein, the term “negatively charged amino acid” includes any amino acids having a negatively charged side chains under normal physiological conditions. Examples of negatively charged naturally occurring amino acids are aspartic acid and glutamic acid.

As used herein, the term “hydrophobic amino acid” includes any amino acids having an uncharged, nonpolar side chain that is relatively insoluble in water. Examples of naturally occurring hydrophobic amino acids are alanine, leucine, isoleucine, valine, proline, phenylalanine, tryptophan, and methionine.

As used herein, the term “hydrophilic amino acid” refers to any amino acids having an uncharged, polar side chain that is relatively soluble in water. Examples of naturally occurring hydrophilic amino acids are serine, threonine, tyrosine, asparagine, glutamine and cysteine.

As used herein, the term “hydrogen bond” refers to two hydrophilic atoms (either O or N), which share a hydrogen that is covalently bonded to only one atom, while interacting with the other.

As used herein, the term “hydrophobic interaction” refers to interactions made by two hydrophobic residues or atoms (such as C).

As used herein, the term “conjugated system” refers to more than two double bonds are adjacent to each other, in which electrons are completely delocalized with the entire system. This also includes aromatic residues.

As used herein, the term “aromatic residue” refers to amino acids with side chains having a delocalized conjugated system. Examples of aromatic residues are phenylalanine, tryptophan, and tyrosine.

As used herein, the terms “c-fms chimera,” “c-fms chimeric polypeptide” and “c-fms chimeric protein” are used interchangeably unless a different meaning is specifically indicated otherwise. When a different meaning is intended, such will be clear from the text. The term “c-fms,” “c-fms protein” and “c-fms polypeptide” may refer to either the chimeric or non-chimeric c-fms. When a different meaning is intended, such will be clear from the text.

As used herein, the term “inhibitor” or “potential inhibitor” means a substance that is believed to interact with another moiety, for example a given ligand that is believed to interact to at least partially inhibit the activity of a complete c-fms or a chimeric c-fms polypeptide, or fragment of either, and which can be subsequently evaluated for such an interaction and inhibititory effect. Representative candidate compounds or substrates include drugs and other therapeutic agents, carcinogens and environmental pollutants, natural products and extracts, as well as steroids, fatty acids and prostaglandins. Other examples of potential inhibitors that can be investigated using the methods of the present invention include, but are not restricted to, agonists and antagonists of c-fms, a chimeric c-fms polypeptide, toxins and venoms, viral epitopes, hormones, hormone receptors, peptides, enzymes, enzyme substrates, co-factors, lectins, sugars, oligonucleotides or nucleic acids, oligosaccharides, proteins, small molecules and monoclonal antibodies. See, for example, U.S. Patent Appl. No. 20040002145.

As used herein, the phrase “inhibiting the binding” refers to preventing or reducing the direct or indirect association of one or more molecules, peptides, proteins, enzymes, or receptors, or preventing or reducing the normal activity of one or more molecules, peptides, proteins, enzymes or receptors, e.g., preventing or reducing the direct or indirect association with c-fms chimeric polypeptides.

As used herein, the term “competitive inhibitor” refers to inhibitors that bind to c-fms chimeras at the same sites as its binding partner(s), thus directly competing with them. Competitive inhibition may, in some instances, be reversed completely by increasing the substrate concentration.

As used herein, the term “uncompetitive inhibitor” refers to one that inhibits the functional activity of a c-fms chimera by binding to a different site than does its substrate(s).

As used herein, the term “non-competitive inhibitor” refers to one that can bind to either the free or bound form of a c-fms chimera.

As used herein the term “irreversible” or “covalent” inhibitor refers to one that inhibits a c-fms chimera by forming a covalent bond with the chimera and either inhibiting the enzyme by excluding its substrate or causing a permanent reorientation of catalytic residues thus rendering the enzyme inactive. Those of skill in the art may identify inhibitors as competitive, uncompetitive, or non-competitive by computer fitting enzyme kinetic data using standard methods. See, for example, Segel, I. H., Enzyme Kinetics, J. Willey & Sons, (1975). Examples of irreversible inhibition are found, for example, in U.S. Pat. Nos. 6,153,617; 6,127,374; 5,4981,616; 5,298,508 and 5,082,964.

As used herein, the term “R or S-isomer” refers to two possible stereroisomers of a chiral carbon according to the Cahn-Ingold-Prelog system adopted by International Union of Pure and Applied Chemistry (IUPAC). Each group attached to the chiral carbon is first assigned to a preference or priority a, b, c or d on the basis of the atomic number of the atom that is directly attached to the chiral carbon. The group with the highest atomic number is given the highest preference a, the group with next highest atomic number is given the next highest preference b; and so on. The group with the lowest preference (d) is then directed away from the viewer. If the trace of a path from a to b to c is counter clockwise, the isomer is designated (S); in the opposite direction, clockwise, the isomer is designated (R).

As used herein, the term “ligand” refers to any molecule, or chemical entity which binds with or to a c-fms chimera, a subunit of a c-fms chimera, a domain of c-fms chimera, a target structual motif of a c-fms chimera or a fragment of a c-fms chimera. Thus, ligands include, but are not limited to, small molecule inhibitors, for example.

The term “soaking in a ligand” or “soaking a ligand” or “soaking” in the context of protein crystallography/structure based drug design refers to a process by which a ligand is brought in contact with and preferably bound to a protein present in crystalline form through diffusion of the ligand through the crystalline matrix. In a typical application a crystal of the protein of interest is placed in a stabilization solution for a certain period of time (hours or days) in which a molar excess of ligand of interest has been at least partially solubilized. Typically the protein is present in unliganded form to facilitate ligand binding but could also be present in complex with a weaker or equally strong ligand as the one one seeks to replace. If the binding affinity of the ligand is high enough and the ligand-binding-site is unobstructed the ligand will bind to the crystalline protein thus enabling the 3-dimensional structure of the protein-ligand complex to be determined by X-ray crystallography.

As used herein, the term “small molecule inhibitor” refers to compounds useful in the present invention having measurable or inhibiting activity. In addition to small organic molecules, peptides, antibodies, cyclic peptides and peptidomimetics are contemplated as being useful in the disclosed methods. Preferred inhibitors are small molecules, preferably less than 700 Daltons, and more preferably less than 450 Daltons.

As used herein, the terms “bind,” “binding,” “bond,” or “bonded” when used in reference to the association of atoms, molecules, or chemical groups, refer to any physical contact or association of two or more atoms, molecules, or chemical groups.

As used herein, the terms “covalent bond” or “valence bond” refer to a chemical bond between two atoms in a molecule created by the sharing of electrons, usually in pairs, by the bonded atoms.

As used herein, “noncovalent bond” refers to an interaction between atoms and/or molecules that does not involve the formation of a covalent bond between them.

As used herein, the term “native protein” refers to a protein comprising an amino acid sequence identical to that of a protein isolated from its natural source or organism.

### SPECIFIC EMBODIMENTS

### Detailed Embodiments

Included in this invention is a substitution of the native kinase insert domain of c-fms, which due to its structural bulk and potential disorder, prevents crystallization of the native protein. The invention also includes a method for replacing the native kinase insert domain with shorter kinase insert domains from the FGF receptor kinase, the tie-2 kinase and the insulin receptor kinase, and obtaining crystals of a c-fms-chimeric protein.

A. Modeling the Three-Dimensional Structure of the c-fms Chimeric Protein

The atomic coordinate data provided in Tables 1, 2 or 3 or the coordinate data derived from homologous proteins may be used to build a three-dimensional model of a c-fms chimeric protein. Any available computational methods may be used to build the three dimensional model. As a starting point, the X-ray diffraction pattern obtained from the assemblage of the molecules or atoms in a crystalline version of a c-fms chimera or a c-fms chimeric homolog can be used to build an electron density map using tools well known to those skilled in the art of crystallography and X-ray diffraction techniques. Additional phase information extracted either from the diffraction data and available in the published literature and/or from supplementing experiments may then used to complete the reconstruction.

For basic concepts and procedures of collecting, analyzing, and utilizing X-ray diffraction data for the construction of electron densities see, for example, Campbellet al., 1984, Biological Spectroscopy, The Benjamin/Cummings Publishing Co., Inc., Menlo Park, Calif.; Cantor et al., 1980, Biophysical Chemistry, Part II: Techniques for the study of biological structure and function, W.H. Freeman and Co., San Francisco, Calif.; A. T. Brunger, 1993, X-Flor Version 3.1: A system for X-ray crystallography and NMR, Yale Univ. Pr., New Haven, Conn.; M. M. Woolfson, 1997, An Introduction to X-ray Crystallography, Cambridge Univ. Pr., Cambridge, UK; J. Drenth, 1999, Principles of Protein X-ray Crystallography (Springer Advanced Texts in Chemistry), Springer Verlag; Berlin; Tsirelson et al., 1996, Electron Density and Bonding in Crystals: Principles, Theory and X-ray Diffraction Experiments in Solid State Physics and Chemistry, Inst. of Physics Pub.; U.S. Pat. No. 5,942,428; U.S. Pat. No. 6,037,117; U.S. Pat. No. 5,200,910 and U.S. Pat. No. 5,365,456 (“Method for Modeling the Electron Density of a Crystal”), each of which is herein specifically incorporated by reference in their entirety.

For basic information on molecular modeling, see, for example, M. Schlecht, Molecular Modeling on the PC, 1998, John Wiley & Sons; Gans et al., Fundamental Principals of Molecular Modeling, 1996, Plenum Pub. Corp.; N. C. Cohen (editor), Guidebook on Molecular Modeling in Drug Design, 1996, Academic Press; and W. B. Smith, Introduction to Theoretical Organic Chemistry and Molecular Modeling, 1996. U.S. patents which provide detailed information on molecular modeling include U.S. Pat. Nos. 6,093,573; 6,080,576; 6,075,014; 6,075,123; 6,071,700; 5,994,503; 5,612,894; 5,583,973; 5,030,103; 4,906,122; and 4,812,12, each of which are incorporated by reference herein in their entirety.

B. Methods of Using the Atomic Coordinates to Identify and Design Ligands of Interest

The atomic coordinates of the invention, such as those described in Tables 1, 2 or 3 or coordinates substantially identical to or homologous to those of Tables 1, 2 or 3 may be used with any available methods to prepare three dimensional models of c-fms chimeras as well as to identify and design ligands, inhibitors or antagonists or agonist molecules.

For instance, three-dimensional modeling may be performed using the experimentally determined coordinates derived from X-ray diffraction patterns, such as those in Tables 1, 2 or 3, for example, wherein such modeling includes, but is not limited to, drawing pictures of the actual structures, building physical models of the actual structures, and determining the structures of related subunits and /ligand and subunit/ligand complexes using the coordinates. Such molecular modeling can utilize known X-ray diffraction molecular modeling algorithms or molecular modeling software to generate atomic coordinates corresponding to the three-dimensional structure of c-fms chimeras.

As described above, molecular modeling involves the use of computational methods, preferably computer assisted methods, to build realistic models of molecules that are identifiably related in sequence to the known crystal structure. It also involves modeling new small molecule inhibitors bound to c-fms chimeras starting with the structures of c-fms chimeras alone or complexed with known ligands or inhibitors. The methods utilized in ligand modeling range from molecular graphics (i.e., 3D representations) to computational chemistry (i.e., calculations of the physical and chemical properties) to make predictions about the binding of ligands or activities of ligands; to design new ligands; and to predict novel molecules, including ligands such as drugs, for chemical synthesis, collectively referred to as rational drug design.

One approach to rational drug design is to search for known molecular structures that might bind to an active site. Using molecular modeling, rational drug design programs can look at a range of different molecular structures of drugs that may fit into the active site of an enzyme or protein, and by moving them in a three-dimensional environment it can be decided which structures actually fit the site well. See, also, for example, data in Tables 1, 2 or 3. An alternate but related rational drug design approach starts with the known structure of a complex with a small molecule ligand and models modifications of that small molecule in an effort to make additional favorable interactions with c-fms chimeras and c-fms proteins.

The present invention includes the use of molecular and computer modeling techniques to design and select ligands, such as small molecule agonists or antagonists or other therapeutic agents that interact with c-fms chimeras as proteins. Such agents include, but are not limited to arylamides and quinolones and derivatives thereof. For example, the invention as herein described includes the design of ligands that act as partial or complete inhibitors of at least one function by binding to all, or a portion of, the active sites or other regions of c-fms chimeras or proteins.

This invention also includes the design of compounds that act as uncompetitive inhibitors of at least one function of c-fms chimeras or proteins. These inhibitors may bind to all, or a portion of, the active sites or other regions of the chimeras or proteins already bound to a ligand and may be more potent and less non-specific than competitive inhibitors that compete for active sites. Similarly, non-competitive inhibitors that bind to and inhibit at least one function of c-fms chimeras or proteins whether or not it is bound to another chemical entity, such as a natural ligand, for example, may be designed using the atomic coordinates of the chimeras or complexes comprising the chimeras of this invention.

The atomic coordinates of the present invention also provide the needed information to probe a crystal of a c-fms chimera with molecules composed of a variety of different chemical features to determine optimal sites for interaction between candidate inhibitors and/or activators and c-fms chimeras. For example, high resolution X-ray diffraction data collected from crystals saturated with solvent allows the determination of where each type of solvent molecule sticks. Small molecules that bind to those sites can then be designed and synthesized and tested for their inhibitory activity (Travis, J., Science 262:1374 (1993)).

The present invention also includes methods for computationally screening small molecule databases and libraries for chemical entities, agents, ligands, or compounds that can bind in whole, or in part, to c-fms chimeras. In this screening, the quality of fit of such entities or compounds to the binding site or sites may be judged either by shape complementarity or by estimated interaction energy (Meng, E. C. et al., J. Comp. Chem. 13:505-524 (1992)).

The design of compounds that bind to, promote or inhibit the functional activity of c-fms proteins and/or chimeras according to this invention generally involves consideration of two factors. First, the compound must be capable of physically and structurally associating with the c-fms protein and/or c-fms chimera. Non-covalent molecular interactions important in the association of the c-fms protein with the compound include hydrogen bonding, van der Waals and hydrophobic interactions. Second, the compound must be able to assume a conformation that allows it to associate with a c-fms protein and/or chimera. Although certain portions of the compound may not directly participate in the association with c-fms, those portions may still influence the overall conformation of the molecule. This, in turn, may have a significant impact on binding affinities, therapeutic efficacy, drug-like qualities and potency. Such conformational requirements include the overall three-dimensional structure and orientation of the chemical entity or compound in relation to all or a portion of the active site or other region of c-fms, or the spacing between functional groups of a compound comprising several chemical entities that directly interact with c-fms.

The potential, predicted, inhibitory agonist, antagonist or binding effect of a ligand or other compound on a c-fms protein may be analyzed prior to its actual synthesis and testing by the use of computer modeling techniques. If the theoretical structure of the given compound suggests insufficient interaction and association between it and the c-fms protein, synthesis and testing of the compound may be obviated. However, if computer modeling indicates a strong interaction, the molecule may then be synthesized and tested for its ability to interact with c-fms. In this manner, synthesis of inoperative compounds may be avoided. In some cases, inactive compounds are synthesized predicted on modeling and then tested to develop a SAR (structure-activity relationship) for compounds interacting with a specific region of a c-fms protein.

One skilled in the art may use one of several methods to screen chemical entities fragments, compounds, or agents for their ability to associate with a c-fms protein and more particularly with the individual binding pockets or active sites of the c-fms protein. This process may begin by visual inspection of, for example, the active site based on the atomic coordinates of the chimeric protein or the chimeric protein complexed with a ligand. Selected chemical entities, compounds, or agents may then be positioned in a variety of orientations, or docked within an individual binding pocket of the chimeric c-fms protein. Docking may be accomplished using software-such as Quanta and Sybyl, followed by energy minimization and molecular dynamics with standard molecular mechanics forcefields, such as CHARMM and AMBER.

Specialized computer programs may also assist in the process of selecting chemical entities. These include but are not limited to: GRID (Goodford, P. J., “A Computational Procedure for Determining Energetically Favorable Binding Sites on Biologically Important Macromolecules,” J. Med. Chem. 28:849-857 (1985), available from Oxford University, Oxford, UK); MCSS (Miranker, A. and M. Karplus, “Functionality Maps of Binding Sites: A Multiple Copy Simultaneous Search Method.” Proteins: Structure, Function and Genetics 11: 29-34 (1991), available from Molecular Simulations, Burlington, Mass.); AUTODOCK (Goodsell, D. S. and A. J. Olsen, “Automated Docking of Substrates to Proteins by Simulated Annealing” Proteins: Structure. Function, and Genetics 8:195-202 (1990), available from Scripps Research Institute, La Jolla, Calif.); DOCK (Kuntz, I. D. et al., “A Geometric Approach to Macromolecule-Ligand Interactions,” J. Mol. Biol. 161:269-288 (1982), available from University of California, San Francisco, Calif.); Gold (Jones, G. et al., “Development and validation of a genetic algorithm for flexible docking.” J. Mol. Biol. 267: 727-748 (1997)); Glide (Halgren, T. A. et al., “Glide: a new approach for rapid, accurate docking and scoring. 2. Enrichment factors in database screening.” J Med Chem, 47:1750-1759 (2004), Friesner, R. A. et al., “Glide: a new approach for rapid, accurate docking and scoring. 1. Method and assessment of docking accuracy.” J Med Chem, 47:1739-1749 (2004)); FlexX (Rarey, M. et al., “A fast flexible docking method using an incremental construction algorithm.” J. Mol. Biol. 261: 470-489 (1996)); and ICM (Abagyan, R. A. and Totrov, M. M., J. Mol. Biol. 235: 983-1002 (1994)).

The use of software such as GRID, a program that determines probable interaction sites between probes with various functional group characteristics and the macromolecular surface, is used to analyze the surface sites to determine structures of similar inhibiting proteins or compounds. The GRID calculations, with suitable inhibiting groups on molecules (e.g., protonated primary amines) as the probe, are used to identify potential hotspots around accessible positions at suitable energy contour levels. The program DOCK may be used to analyze an active site or ligand binding site and suggest ligands with complementary steric properties. See also, See, also, Kellenberger, P. N et al., “Recovering the true targets of specific ligands by virtual screening of the protein data bank,” Proteins 54(4):671-80 (2004); Oldfield, T., “Applications for macromolecular map interpretation: X-AUTOFIT, X-POWERFIT, X-BUILD, X-LIGAND, and X-SOLVATE,” Methods Enzymol. 374:271-300 (2003); Richardson, J. S. et al., “New tools and data for improving structures, using all-atom contacts,” Methods Enzymol. 374: 385-412 (2003); Terwilliger, T. C., “Improving macromolecular atomic models at moderate resolution by automated iterative model building, statistical density modification and refinement,” Acta Crystallogr D Biol Crystallogr. 59(Pt 7): 1174-82 (2003); Toerger, T. C. and Sacchettini, J. C., “TEXTAL system: artificial intelligence techniques for automated protein model building,” Methods Enzymol. 374:244-70 (2003); von Grotthuss, M. et al., “Predicting protein structures accurately,” Science 304(5677):1597-9 (2004); Rajakiannan, V. et al., “The use of ACORN in solving a 39.5 kDa macromolecule with 1.9 Å resolution laboratory source data,” J Synchrotron Radiat. 11(Pt 4):358-62 (2004); Claude, J. B. et al., “CaspR: a web server for automated molecular replacement using homology modelling,” Nucleic Acids Res. 32(Web Server issue):W606-9 (2004); Suhre, K. and Sanejouand, Y. H., “ElNemo: a normal mode web server for protein movement analysis and the generation of templates for molecular replacement,” Nucleic Acids Res. 32(Web Server issue):W610-4 (2004).

Once suitable chemical entities, compounds, or agents have been selected, they can be assembled into a single ligand or compound or inhibitor or activator. Assembly may proceed by visual inspection of the relationship of the fragments to each other on the three-dimensional image. This may be followed by manual model building using software such as Quanta or Sybyl.

Useful programs to aid in connecting the individual chemical entities, compounds, or agents include but are not limited to: CAVEAT (Bartlett, P. A. et al., “CAVEAT: A Program to Facilitate the Structure-Derived Design of Biologically Active Molecules.” In Molecular Recognition in Chemical and Biological, Problems, Special Pub., Royal Chem. Soc., 78, pp. 82-196 (1989)); 3D Database systems such as MACCS-3D (MDL Information Systems, San Leandro, C A and Martin, Y. C., “3D Database Searching in Drug Design,” J. Med. Chem. 35: 2145-2154 (1992); and HOOK (available from Molecular Simulations, Burlington, Mass.).

Several methodologies for searching three-dimensional databases to test pharmacophore hypotheses and select compounds for screening are available. These include the program CAVEAT (Bacon et al., J. Mol. Biol. 225:849-858 (1992)). For instance, CAVEAT uses databases of cyclic compounds which can act as “spacers” to connect any number of chemical fragments already positioned in the active site. This allows one skilled in the art to quickly generate hundreds of possible ways to connect the fragments already known or suspected to be necessary for tight binding.

Instead of proceeding to build an inhibitor activator, agonist or antagonist of a c-fms chimeric protein in a step-wise fashion one chemical entity at a time as described above, such compounds may be designed as a whole or “de novo” using either an empty active site or optionally including some portion(s) of a known molecules. These methods include: LUDI (Bohm, H.-J., “The Computer Program LUDI: A New Method for the De Novo Design of Enzyme Inhibitors”, J. Comp. Aid. Molec. Design, 6, pp. 61-78 (1992), available from Biosym Technologies, San Diego, Calif.); LEGEND (Nishibata, Y. and A. Itai, Tetrahedron 47:8985 (1991), available from Molecular Simulations, Burlington, Mass.); and LeapFrog (available from Tripos Associates, St. Louis, Mo.).

For instance, the program LUDI can determine a list of interaction sites into which to place both hydrogen bonding and hydrophobic fragments. LUDI then uses a library of linkers to connect up to four different interaction sites into fragments. Then smaller “bridging” groups such as —CH2— and —COO— are used to connect these fragments. For example, for the enzyme DHFR, the placements of key functional groups in the well-known inhibitor methotrexate were reproduced by LUDI. See also, Rotstein and Murcko, J. Med. Chem. 36: 1700-1710 (1992).

Other molecular modeling techniques may also be employed in accordance with this invention. See, e.g., Cohen, N. C. et al., “Molecular Modeling Software and Methods for Medicinal Chemistry, J. Med. Chem. 33:883-894 (1990). See also, Navia, M. A. and M. A. Murcko, “The Use of Structural Information in Drug Design,” Current Opinions in Structural Biology, 2, pp. 202-210 (1992).

Once a compound has been designed or selected by the above methods, the affinity with which that compound may bind or associate with a c-fms protein may be tested and optimized by computational evaluation and/or by testing biological activity after synthesizing the compound. Inhibitors or compounds may interact with c-fms in more than one conformation that is similar in overall binding energy. In those cases, the deformation energy of binding is taken to be the difference between the energy of the free compound and the average energy of the conformations observed when the compound binds to a c-fms protein.

A compound designed or selected as binding or associating with c-fms may be further computationally optimized so that in its bound state it would preferably lack repulsive electrostatic interaction with the protein. Such non-complementary (e.g., electrostatic) interactions include repulsive charge-charge, dipole-dipole and charge-dipole interactions. Specifically, the sum of all electrostatic interactions between the inhibitor and the chimera when the inhibitor is bound, preferably make a neutral or favorable contribution to the enthalpy of binding. Weak binding compounds will also be designed by these methods so as to determine SAR.

Specific computer software is available in the art to evaluate compound deformation energy and electrostatic interaction. Examples of programs designed for such uses include: Gaussian 92, revision C (M. J. Frisch, Gaussian, Inc., Pittsburgh, Pa., COPYRGT 1992); AMBER, version 4.0 (P. A. Kollman, University of California at San Francisco, COPYRGT 1994); QUANTA/CHARMM (Molecular Simulations, Inc., Burlington, Mass. COPYRGT 1994); Insight II/Discover (Biosysm Technologies Inc., San Diego, Calif. COPYRGT. 1994); and Delphi (A. Nicholls and B. Honig “A rapid finite difference algorithm, utilizing successive over-relaxation to solve the Poisson-Boltzman equation” J. Comp. Chem. 12: 435-445 (1991), M. K. Gilson and B. Honig. “Calculation of the total electrostatic energy of a macromolecular system: Solvation energies, binding energies and conformation analysis” Proteins 4: 7-18 (1988), M. K. Gilson et al., “Calculating the electrostatic potential of molecules in solution: Method and error assessment” J Comp. Chem 9: 327-335 (1987)). Other hardware systems and software packages will be known to those skilled in the art.

Once a compound that associates with the c-fms chimera and/or c-fms protein has been optimally selected or designed, as described above, substitutions may then be made in some of its atoms or side groups in order to improve or modify its binding properties. Generally, initial substitutions are conservative, i.e., the replacement group will have approximately the same size, shape, hydrophobicity and charge as the original group. It should, of course, be understood that components known in the art to alter conformation may be avoided. Such substituted chemical compounds may then be analyzed for efficiency of fit to a c-fms chimera by the same computer methods described in detail, above.

C. Use of Homology Structure Modeling to Design Ligands with Modulated Binding or Activity to c-fms Proteins

The present invention includes the use of the atomic coordinates and structures of c-fms chimeric proteins and/or c-fms chimeric protein inhibitor complexes. The structure of a complex between the chimera and the starting compound can be used to guide the modification of that compound to produce new compounds that have other desirable properties for applicable industrial and other uses (e.g., as pharmaceuticals), such as chemical stability, solubility or membrane permeability (Lipinski et al., Adv. Drug Deliv. Rev. 23:3 (1997)).

Binding compounds, agonists, antagonists and such that are known in the art include but are not limited to arylamides and quinolones. Such compounds can be diffused into or soaked with the stabilized crystals of a c-fms chimera to form a complex for collecting X-ray diffraction data. Alternatively, other compounds, known and unknown in the art, can be cocrystallized with the c-fms chimera by mixing the compound with the chimera before precipitation.

To produce custom high affinity and very specific compounds, the structure of a c-fms chimera can be compared to the structure of a selected non-targeted molecule and a hybrid constructed by changing the structure of residues at the binding site for a ligand for the residues at the same positions of the non-target molecule. The process whereby this modeling is achieved is referred to as homology structure modeling. This is done computationally by removing the side chains from the molecule or target of known structure and replacing them with the side chains of the non-targeted structure put in sterically plausible positions. In this way it can be understood how the shapes of the active site cavities of the targeted and non-targeted molecules differ. This process, therefore, provides information concerning how a bound ligand can be chemically altered in order to produce compounds that will bind tightly and specifically to the desired target but will simultaneously be sterically prevented from binding to the non-targeted molecule. Likewise, knowledge of portions of the bound ligands that are facing to the solvent would allow introduction of other functional groups for additional pharmaceutical purposes. The use of homology structure modeling to design molecules (ligands) that bind more tightly to the target enzyme than to the non-target enzyme has wide spread applicability.

D. High Throughput Assays

Any high throughput screening may be utilized to test new compounds which are identified or designed for their ability to interact with c-fms. For general information on high-throughput screening see, for example, Devlin, 1998, High Throughput Screening, Marcel Dekker; and U.S. Pat. No. 5,763,263. High throughput assays utilize one or more different assay techniques including, but not limited to, those described below.

Immunodiagnostics and Immunoassays. These are a group of techniques used for the measurement of specific biochemical substances, commonly at low concentrations in complex mixtures such as biological fluids, that depend upon the specificity and high affinity shown by suitably prepared and selected antibodies for their complementary antigens. A substance to be measured must, of necessity, be antigenic—either an immunogenic macromolecule or a haptenic small molecule. To each sample a known, limited amount of specific antibody is added and the fraction of the antigen combining with it, often expressed as the bound:free ratio, is estimated, using as indicator a form of the antigen labeled with radioisotope (radioimmunoassay), fluorescent molecule (fluoroimmunoassay), stable free radical (spin immunoassay), enzyme (enzyme immunoassay), or other readily distinguishable label.

Antibodies can be labeled in various ways, including: enzyme-linked immunosorbent assay (ELISA); radioimmuno assay (RIA); fluorescent immunoassay (FIA); chemiluminescent immunoassay (CLIA); and labeling the antibody with colloidal gold particles (immunogold). Common assay formats include the sandwich assay, competitive or competition assay, latex agglutination assay, homogeneous assay, microtitre plate format and the microparticle-based assay.

Enzyme-linked immunosorbent assay (ELISA). ELISA is an immunochemical technique that avoids the hazards of radiochemicals and the expense of fluorescence detection systems. Instead, the assay uses enzymes as indicators. ELISA is a form of quantitative immunoassay based on the use of antibodies (or antigens) that are linked to an insoluble carrier surface, which is then used to “capture” the relevant antigen (or antibody) in the test solution. The antigen-antibody complex is then detected by measuring the activity of an appropriate enzyme that had previously been covalently attached to the antigen (or antibody).

For information on ELISA techniques, see, for example, Crowther, (1995) ELISA—Theory and Practice (Methods in Molecular Biology), Humana Press; Challacombe & Kemeny, (1998) ELISA and Other Solid Phase Immunoassays—Theoretical and Practical Aspects, John Wiley; Kemeny, (1991) A Practical Guide to ELISA, Pergamon Press; Ishikawa, (1991) Ultrasensitive and Rapid Enzyme Immunoassay (Laboratory Techniques in Biochemistry and Molecular Biology) Elsevier.

Colorimetric Assays for Enzymes. Colorimetry is any method of quantitative chemical analysis in which the concentration or amount of a compound is determined by comparing the color produced by the reaction of a reagent with both standard and test amounts of the compound, often using a colorimeter. A colorimeter is a device for measuring color intensity or differences in color intensity, either visually or photoelectrically.

Standard calorimetric assays of beta-galactosidase enzymatic activity are well known to those skilled in the art (see, for example, Norton et al., Mol. Cell. Biol. 5:281-290 (1985)). A calorimetric assay can be performed on whole cell lysates using O-nitrophenyl -beta-D-galactopyranoside (ONPG, Sigma) as the substrate in a standard colorimetric beta-galactosidase assay (Sambrook et al., (1989) Molecular Cloning—A Laboratory Manual, Cold Spring Harbor Laboratory Press). Automated calorimetric assays are also available for the detection of beta-galactosidase activity, as described in U.S. Pat. No. 5,733,720.

Immunofluorescence Assays. Immunofluorescence or immunofluorescence microscopy is a technique in which an antigen or antibody is made fluorescent by conjugation to a fluorescent dye and then allowed to react with the complementary antibody or antigen in a tissue section or smear. The location of the antigen or antibody can then be determined by observing the fluorescence by microscopy under ultraviolet light.

For general information on immunofluorescent techniques, see, for example, Knapp et al., (1978) Immunofluorescence and Related Staining Techniques, Elsevier; Allan, (1999) Protein Localization by Fluorescent Microscopy—A Practical Approach (The Practical Approach Series) Oxford University Press; Caul, (1993) Immunofluorescence Antigen Detection Techniques in Diagnostic Microbiology, Cambridge University Press. For detailed explanations of immunofluorescent techniques applicable to the present invention, see U.S. Pat. No. 5,912,176; U.S. Pat. No. 5,869,264; U.S. Pat. No. 5,866,319; and U.S. Pat. No. 5,861,259.

E. Databases and Computer Systems

An amino acid sequence or nucleotide sequence of a c-fms chimera and/or X-ray diffraction data, useful for computer molecular modeling of a c-fms chimera or a portion thereof, can be “provided” in a variety of mediums to facilitate use thereof. As used herein, “provided” refers to a manufacture, which contains, for example, an amino acid sequence or nucleotide sequence and/or atomic coordinates derived from X-ray diffraction data of the present invention, e.g., an amino acid or nucleotide sequence of a c-fms chimera, a representative fragment thereof, or a homologue thereof. Such a product provides the amino acid sequence and/or X-ray diffraction data in a form which allows a skilled artisan to analyze and molecular model the three-dimensional structure of a c-fms chimera or related molecules, including a subdomain thereof.

In one application of this embodiment, databases comprising data pertaining to a c-fms chimera, or at least one subdomain thereof, amino acid and nucleic acid sequence and/or X-ray diffraction data of the present invention is recorded on computer readable medium. As used herein, “computer readable medium” refers to any medium which can be read and accessed directly by a computer. Such media include, but are not limited to: magnetic storage media, such as floppy discs, hard disc storage media, and magnetic tape; optical storage media such as optical discs or CD-ROM; electrical storage media such as RAM and ROM; and hybrids of these categories such as magnetic/optical storage media. A skilled artisan can readily appreciate how any of the presently known computer readable media can be used to create a manufacture comprising computer readable medium having recorded thereon an amino acid sequence and/or X-ray diffraction data of the present invention.

As used herein, “recorded” refers to a process for storing information on computer readable media. A skilled artisan can readily adopt any of the presently known methods for recording information on computer readable media to generate manufactures comprising an amino acid sequence and/or atomic coordinate/X-ray diffraction data information of the present invention.

A variety of data storage structures are available to a skilled artisan for creating a computer readable medium having recorded thereon an amino acid sequence and/or atomic coordinate/X-ray diffraction data of the present invention. The choice of the data storage structure will generally be based on the means chosen to access the stored information. In addition, a variety of data processor programs and formats can be used to store the sequence and X-ray data information of the present invention on computer readable media. The sequence information can be represented in a word processing text file, formatted in commercially-available software such as WordPerfect and MICROSOFT Word, or represented in the form of an ASCII file, stored in a database application, such as DB2, Sybase, Oracle, or the like. A skilled artisan can readily adapt any number of dataprocessor structuring formats (e.g., text file or database) in order to obtain computer readable media having recorded thereon the information of the present invention.

By providing computer readable media having sequence and/or atomic coordinates based on X-ray diffraction data, a skilled artisan can routinely access the sequence and atomic coordinate or X-ray diffraction data to model, for instance, a related molecule, a subdomain, mimetic, or a ligand thereof. Computer algorithms are publicly and commercially available which allow a skilled artisan to access this data provided in a computer readable medium and analyze it for molecular modeling and/or RDD (rational drug design). See, e.g., Biotechnology Software Directory, MaryAnn Liebert Publ., New York (1995).

The present invention further provides systems, particularly computer-based systems, which contain the sequence, structure, and/or diffraction data described herein. Such systems are designed to do structure determination and RDD for a c-fms chimera or at least one subdomain thereof. Non-limiting examples are microcomputer workstations available from Silicon Graphics Incorporated and Sun Microsystems running UNIX based, Windows or IBM OS/2 operating systems.

As used herein, “a computer-based system” refers to the hardware means, software means, and data storage means used to analyze the sequence, structure, and/or X-ray diffraction data of the present invention. The minimum hardware means of the computer-based systems of the present invention comprises a central processing unit (CPU), input means, output means, and data storage means. A skilled artisan can readily appreciate which of the currently available computer-based systems are suitable for use in the present invention. A visualization device, such as a monitor, is optionally provided to visualize structure data.

As stated above, the computer-based systems of the present invention comprise a data storage means having stored therein sequence, structure, and/or atomic coordinate/X-ray diffraction data of the present invention and the necessary hardware means and software means for supporting and implementing an analysis means. As used herein, “data storage means” refers to memory which can store sequence, structure, or atomic coordinate/X-ray diffraction data of the present invention, or a memory access means which can access manufactures having recorded thereon the sequence or X-ray data of the present invention.

As used herein, “search means” or “analysis means” refers to one or more programs which are implemented on the computer-based system to compare a target sequence or target structural motif with the sequence, structure, or X-ray data stored within the data storage means. Search means are used, for instance, to identify fragments or regions of a protein or polypeptide which match a particular target sequence or target motif. A variety of known algorithms are disclosed publicly and a variety of commercially available software for conducting search means are and can be used in the computer-based systems of the present invention. A skilled artisan can readily recognize that any one of the available algorithms or implementing software packages for conducting computer analyses can be adapted for use in the present computer-based systems.

As used herein, “a target structural motif,” or “target motif,” refers to any rationally selected sequence or combination of sequences in which the sequence(s) are chosen based on a three-dimensional configuration or electron density map which is formed upon the folding of the target motif. There are a variety of target motifs known in the art. Protein target motifs include, but are not limited to, enzymatic active sites, inhibitor binding sites, structural subdomains, epitopes, functional domains and signal sequences. Similar motifs are known for RNA. A variety of structural formats for the input and output means can be used to input and output the information in the computer-based systems of the present invention.

A variety of comparing means can be used to compare a target sequence or target motif with the data described herein to identify structural motifs or electron density maps derived in part from the atomic coordinate/X-ray diffraction data. A skilled artisan can readily recognize that any one of the publicly available computer modeling programs can be used as the search means for the computer-based systems of the present invention.

F. Target Molecule Fragments and Portions

Fragments of c-fms, for instance fragments comprising active sites defined by two or more amino acids selected from the group consisting of: Trp 550, Lys 586, Thr 587, Leu 588, Gly 589, Val 596, Glu 598, Ala 614, Val 615, Lys 616, Glu 633, Met 637, Leu 640, Ile 646, Val 647, Val 661, Thr 663, Glu 664, Tyr 665, Cys 666, Cys 667, Tyr 668, Gly 669, Asp 670, Asn 673, Arg 677, Cys 774, Ile 775, His 776, Arg 782, Asn 783, Leu 785, Ile 794, Gly 795, Asp 796, Phe 797, Gly 798, Leu 799, Ala 800, Arg 801, Asp 802 may be prepared by any available means including synthetic or recombinant means. Such fragments may then be used in the assays as described above, for instance, high through put assays to detect interactions between prospective agents and the active site within the fragment.

For recombinant expression or production of the fragments of the invention, nucleic acid molecules encoding the fragment may be prepared. As used herein, “nucleic acid” is defined as RNA or DNA that encodes a protein or peptide as defined above, or is complementary to nucleic acid sequence encoding such peptides, or hybridizes to such nucleic acid and remains stably bound to it under appropriate stringency conditions.

Nucleic acid molecules encoding fragments of the invention may differ in sequence because of the degeneracy in the genetic code or may differ in sequence as they encode proteins or protein fragments that differ in amino acid sequence. Homology or sequence identity between two or more such nucleic acid molecules is determined by BLAST (Basic Local Alignment Search Tool) analysis using the algorithm employed by the programs blastp, blastn, blastx, tblastn and tblastx (Karlin et al., Proc. Natl. Acad. Sci. USA 87:2264-2268 (1990) and Altschul et al., J. Mol. Evol. 36:290-300 (1993), fully incorporated by reference) which are tailored for sequence similarity searching.

The approach used by the BLAST program is to first consider similar segments between a query sequence and a database sequence, then to evaluate the statistical significance of all matches that are identified and finally to summarize only those matches which satisfy a preselected threshold of significance. For a discussion of basic issues in similarity searching of sequence databases, see Altschul et al. (Nat. Genet. 6, 119-129 (1994)) which is fully incorporated by reference. The search parameters for histogram, descriptions, alignments, expect (i.e., the statistical significance threshold for reporting matches against database sequences), cutoff, matrix and filter are at the default settings. The default scoring matrix used by blastp, blastx, tblastn, and tblastx is the BLOSUM62 matrix (Henikoff et al., Proc. Natl. Acad. Sci. USA 89:10915-10919 (1992), fully incorporated by reference). Four blastn parameters were adjusted as follows: Q=10 (gap creation penalty); R=10 (gap extension penalty); wink=1 (generates word hits at every wink^{th} position along the query); and gapw=16 (sets the window width within which gapped alignments are generated). The equivalent Blastp parameter settings were Q=9; R=2; wink=1; and gapw=32. A Bestfit comparison between sequences, available in the GCG package version 10.0, uses DNA parameters GAP=50 (gap creation penalty) and LEN=3 (gap extension penalty) and the equivalent settings in protein comparisons are GAP=8 and LEN=2.

“Stringent conditions” are those that (1) employ low ionic strength and high temperature for washing, for example, 0.015 M NaCl/0.0015 M sodium citrate/0.1% SDS at 50° C. or (2) employ during hybridization a denaturing agent such as formamide, for example, 50% formamide with 0.1% bovine serum albumin/0.1% Ficoll/0.1% polyvinylpyrrolidone/50 mM, sodium phosphate buffer at pH 6.5 with 750 mM NaCl, 75 mM sodium citrate at 42° C. Another example is use of 50% formamide, 5×SSC, 50 mM sodium phosphate (pH 6.8), 0.1% sodium pyrophosphate, 5× Denhardt's solution, sonicated salmon sperm DNA (50 mg/ml), 0.1% SDS and 10% dextran sulfate at 42° C., with washes at 42° C. in 0.2×SSC and 0.1% SDS. A skilled artisan can readily determine and vary the stringency conditions appropriately to obtain a clear and detectable hybridization signal.

As used herein, a nucleic acid molecule is said to be “isolated” when the nucleic acid molecule is substantially separated from contaminant nucleic acid encoding other polypeptides from the source of nucleic acid.

The encoding nucleic acid molecules of the present invention (i.e., synthetic oligonucleotides) and those that are used as probes or specific primers for polymerase chain reaction (PCR) or to synthesize gene sequences encoding proteins of the invention can easily be synthesized by chemical techniques, for example, the phosphotriester method of Matteucci et al. (J. Am. Chem. Soc. 103: 185-3191 (1981)) or using automated synthesis methods. In addition, larger DNA segments can readily be prepared by well known methods, such as synthesis of a group of oligonucleotides that define various modular segments of the gene, followed by ligation of oligonucleotides to build the complete modified gene.

The encoding nucleic acid molecules of the present invention may further be modified so as to contain a detectable label for diagnostic and probe purposes. A variety of such labels are known in the art and can readily be employed with the encoding molecules herein described. Suitable labels include, but are not limited to, biotin, radiolabeled nucleotides and the like. A skilled artisan can employ any of the art-known labels to obtain a labeled encoding nucleic acid molecule.

The present invention further provides recombinant DNA molecules (rDNA) that contain a coding sequence for a protein fragment as described above. As used herein, a rDNA molecule is a DNA molecule that has been subjected to molecular manipulation. Methods for generating rDNA molecules are well known in the art, for example, see Sambrook et al., Molecular Cloning—A Laboratory Manual, Cold Spring Harbor Laboratory Press (1989). In the preferred rDNA molecules, a coding DNA sequence is operably linked to expression control sequences and/or vector sequences.

The choice of vector and expression control sequences to which one of the protein encoding sequences of the present invention is operably linked depends directly, as is well known in the art, on the functional properties desired (e.g., protein expression, and the host cell to be transformed). A vector of the present invention may be capable of directing the replication or insertion into the host chromosome, and preferably also expression, of the structural gene included in the rDNA molecule.

Expression control elements that are used for regulating the expression of an operably linked protein encoding sequence are known in the art and include, but are not limited to, inducible promoters, constitutive promoters, secretion signals, and other regulatory elements. Preferably, the inducible promoter is readily controlled, such as being responsive to a nutrient in the host cell's medium.

The present invention further provides host cells transformed with a nucleic acid molecule that encodes a protein, polypeptide, or fragment of a protein or polypeptide of the present invention. The host cell can be either prokaryotic or eukaryotic. Eukaryotic cells useful for expression of a protein of the invention are not limited, so long as the cell line is compatible with cell culture methods and compatible with the propagation of the expression vector and expression of the gene product. Preferred eukaryotic host cells include, but are not limited to, yeast, insect and mammalian cells, preferably vertebrate cells such as those from a mouse, rat, monkey or human cell line. Preferred eukaryotic host cells include Chinese hamster ovary (CHO) cells available from the ATCC as CCL61, NIH Swiss mouse embryo cells NIH-3T3 available from the ATCC as CRL1658, baby hamster kidney cells (BHK), and the like eukaryotic tissue culture cell lines.

Transformed host cells of the invention may be cultured under conditions that allow the production of the recombinant protein. Optionally the recombinant protein is isolated from the medium or from the cells; recovery and purification of the protein may not be necessary in some instances where some impurities may be tolerated.

Kits may also be prepared with any of the above described nucleic acid molecules, protein fragments, vector and/or host cells optionally packaged with the reagents needed for a specific assay, such as those described above. In such kits, the protein fragments or other reagents may be attached to a solid support, such as glass or plastic beads.

G. Integrated Procedures Which Utilize the Present Invention

Molecular modeling is provided by the present invention for rational drug design (RDD) of mimetics and ligands of a c-fms chimera. As described above, the drug design paradigm uses computer modeling programs to determine potential mimetics and ligands of a c-fms chimera which are expected to interact with sites on the protein. The potential mimetics or ligands are then screened for activity and/or binding and/or interaction with the c-fms protein. For c-fms-related mimetics or ligands, screening methods can be selected from assays for at least one biological activity of c-fms, e.g., such as phosphorylation, according to known method steps. See, for example, U.S. Appl. No. 2004/0002145 A1.

Thus, the tools and methodologies provided by the present invention may be used in procedures for identifying and designing ligands which bind in desirable ways with the target, a c-fms protein. Such procedures utilize an iterative process whereby ligands are synthesized, tested and characterized. New ligands can be designed based on the information gained in the testing and characterization of the initial ligands and then such newly identified ligands can themselves be tested and characterized. This series of processes may be repeated as many times as necessary to obtain ligands with the desirable binding properties.

The following steps serve as an example of the overall procedure:

1. A biological activity of a target is selected.

2. A ligand is identified that appears to be in some way associated with the chosen biological activity (e.g., the ligand may be an inhibitor of a known activity). The activity of the ligand may be tested by in vivo and/or in vitro methods.

A ligand of the present invention can be, but is not limited to, at least one selected from a lipid, a nucleic acid, a compound, a protein, an element, an antibody, a saccharide, an isotope, a carbohydrate, an imaging agent, a lipoprotein, a glycoprotein, an enzyme, a detectable probe, and antibody or fragment thereof, or any combination thereof, which can be detectably labeled as for labeling antibodies. Such labels include, but are not limited to, enzymatic labels, radioisotope or radioactive compounds or elements, fluorescent compounds or metals, chemiluminescent compounds and bioluminescent compounds. Alternatively, any other known diagnostic or therapeutic agent can be used in a method of the invention. Suitable compounds are then tested for activities in relationship to the target.

Complexes between a c-fms chimera and ligands are made either by co-crystallization or more commonly by diffusing the small molecule ligand into the crystal. X-ray diffraction data from the complex crystal are measured and a difference electron density map is calculated. This process provides the precise location of the bound ligand on the target molecule. The difference Fourier is calculated using measured diffraction amplitudes and the phases of these reflections calculated from the coordinates.

3. Using the methods of the present invention, X-ray crystallography is utilized to create electron density maps and/or molecular models of the interaction of the ligand with the target molecule.

The entry of the coordinates of the target into the computer programs discussed above results in the calculation of the most probable structure of the macromolecule. These structures are combined and refined by additional calculations using such programs to determine the probable or actual three-dimensional structure of the target including potential or actual active or binding sites of ligands. Such molecular modeling (and related) programs useful for rational drug design of ligands or mimetics, are also provided by the present invention.

4. The electron density maps and/or molecular models obtained in Step 3 are compared to the electron density maps and/or molecular models of a non-ligand containing target and the observed/calculated differences are used to specifically locate the binding of the ligand on the target or subunit.

5. Modeling tools, such as computational chemistry and computer modeling, are used to adjust or modify the structure of the ligand so that it can make additional or different interactions with the target.

The ligand design uses computer modeling programs which calculate how different molecules interact with the various sites of a target. This procedure determines potential ligands or mimetics of the ligand(s).

The ligand design uses computer modeling programs which calculate how different molecules interact with the various sites of the target, subunit, or a fragment thereof. Thus, this procedure determines potential ligands or ligand mimetics.

6. The newly designed ligand from Step 5 can be tested for its biological activity using appropriate in vivo or in vitro tests, including the high throughput screening methods discussed above.

The potential ligands or mimetics are then screened for activity relating to a c-fms chimera, c-fms protein, or at least a fragment thereof. Such screening methods are selected from assays for at least one biological activity of the native target.

The resulting ligands or mimetics, provided by methods of the present invention, are useful for treating, screening or preventing diseases in animals, such as mammals (including humans) and birds.

7. Of course, each of the above steps can be modified as desired by those of skill in the art so as to refine the procedure for the particular goal in mind. Also, additional X-ray diffraction data may be collected on c-fms chimeric proteins, c-fms chimeric proteins/ligand complexes, structural target motifs and subunit/ligand complexes at any step or phase of the procedure. Such additional diffraction data can be used to reconstruct electron density maps and molecular models which may further assist in the design and selection of ligands with the desirable binding attributes.

It is to be understood that the present invention is considered to include stereoisomers as well as optical isomers, e.g., mixtures of enantiomers as well as individual enantiomers and diastereomers, which arise as a consequence of structural asymmetry in selected compounds, ligands or mimetics of the present series.

Some of the compounds or agents disclosed or discovered by the methods herein may contain one or more asymmetric centers and thus give rise to enantiomers, diastereomers, and other stereoisomeric forms. The present invention is also meant to encompass all such possible forms as well as their racemic and resolved forms and mixtures thereof. When the compounds described or discovered herein contain olefinic double bonds or other centers of geometric asymmetry, and unless otherwise specified, it is intended to include both E and Z geometric isomers. All tautomers are intended to be encompassed by the present invention as well.

As used herein, the term “stereoisomers” is a general term for all isomers of individual molecules that differ only in the orientation of their atoms in space. It includes enantiomers and isomers of compounds with more than one chiral center that are not mirror images of one another (diastereomers).

As used herein, the term “chiral center” refers to to a carbon atom to which four different groups are attached.

As used herein, the term “enantiomer” or “enantiomeric” refers to a molecule that is nonsuperimposable on its mirror image and hence optically active wherein the enantiomer rotates the plane of polarized light in one direction and its mirror image rotates the plane of polarized light in the opposite direction.

As used herein, the term “racemic” refers to a mixture of equal parts of enantiomers and which is optically active.

As used herein, the term “resolution” refers to the separation or concentration or depletion of one of the two enantiomeric forms of a molecule. In the context of this application, the term “resolution” also refers to the amount of detail which can be resolved by the diffraction experiment. Or in other terms, since the inherent disorder of a protein crystal diffraction pattern fades away at some diffraction angle θₘₐₓ, the corresponding distance dₘᵢₙ of the reciprocal lattices is deterimined by Bragg's law.

$${d}_{min} = \frac{λ}{2 sin {θ}_{max}}$$

In practice in protein crystallography it is usual to quote the nominal resolution of a protein electron density in terms of dₘᵢₙ, the minimum lattice distance to which data is included in the calculation of the map.

Without further description, it is believed that one of ordinary skill in the art can, using the preceding description and the following illustrative examples, make and utilize the compounds of the present invention and practice the claimed methods. The following working examples therefore, specifically point out preferred embodiments of the present invention, and are not to be construed as limiting in any way the remainder of the disclosure.

### EXAMPLES

### Example 1

### Cloning of c-FMS-FGFR1 Chimera 538-922

All constructs begin at amino acid 538 of FMS and end at amino acid 922 of FMS. Chimeras were created by replacing FMS KID with KID sequences from RTK's known to be structured, like tie2 and irk. Chimeras are based on structure prediction and sequence alignment.

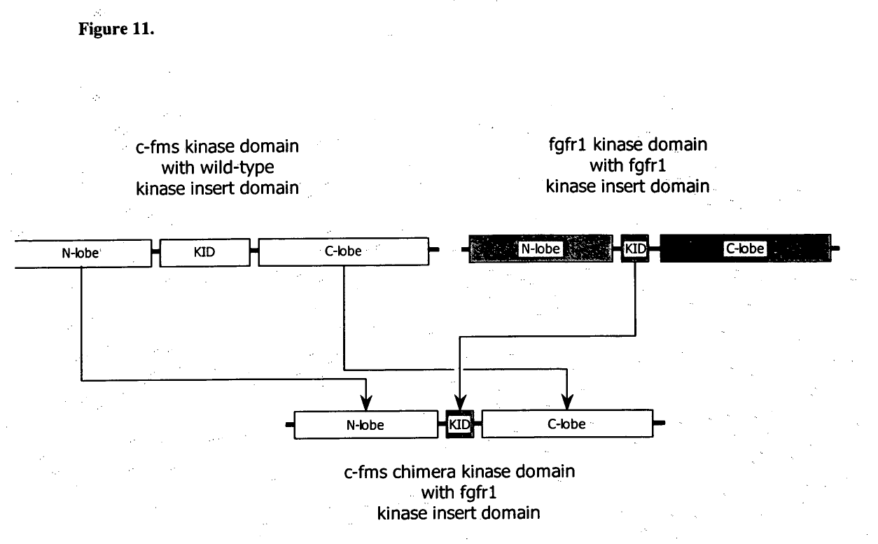

c-fms fragments from amino acids 922-678 and 753-922 were generated by PCR using a c-fms construct derived by RT-PCR from THP-1 cells. For cloning purposes, a Sal I site was included on the 5′ side of the 922-678 PCR product and a stop codon followed by a Not I site was included on the 3′ end of the 753-922 PCR product. The FGFR1 kinase insert domain was generated by annealing 2 synthesized oligonucleotides corresponding to amino acids 671-679 of c-fms, followed by amino acids 577-617 of FGFR1 and ending with amino acids 753-760 of c-fms. To obtain the chimera, overlapping PCR was performed using the FMS PCR fragments 922-678 and 753-922 and the annealed synthesized FGFR1 kinase insert domain oligonucleotides as a template. The final PCR product was subcloned into pCRII (Invitrogen) and the sequence was confirmed. For expression in SF9 cells, a recombinant baculovirus was generated by subcloning the FMS-FGFR1 chimera into a modified Invitrogen GATEWAY pDEST8 vector, and following the protocol for Baculovirus Expression according to the Bac-to-Bac manual. Other chimeras were generated in a similar manner by using synthetic oligonucleotides corresponding to the KID of TIE2 or IR. FIG. 11 shows the Kinase insert domain replaced by FGFR1 KID.

### Example 2

### Protein Purification

Frozen cells were thawed and resuspended in 50 mM NaKPO₄ pH 7.5, 200 mM NaCl, 5% Glycerol, 1 mM Glutathione, 5 mM Imidazole, 1× Complete EDTA-free protease inhibitor cocktail (Roche) (Buffer A). Thawed cells were dounce homogenized, mechanically lysed with an Emulsiflex-C5 (Avestin) at 10,000-15,000 psi and centrifuged at 40,000×g (16,000 rpm) for 1 hour to remove insoluble material. The supernatant was filtered through a 0.45 μm vacuum filter and incubated with a BD Talon metal affinity resin (BD Biosciences Clontech) overnight at 4° C. After 20 column volumes washes with buffer A containing 10 mM Imidazole, c-fms was eluted using a 10 column volume linear gradient from 10 mM to 200 mM Imidazole in buffer A. Fractions containing c-fms, as assayed by SDS-PAGE, were pooled and combined with 0.2 Units of TEV Protease (Invitrogen)/μg of c-fms to remove the histidine tag. The reaction was dialyzed overnight against 50 mM NaKPO₄ pH 7.5, 200 mM NaCl, 5% Glycerol, 2 mM Glutathione and was then incubated with a BD Talon metal affinity resin for two hours to remove TEV protease. Purified c-fms was then filtered through a 0.2 μm filter, concentrated and further purified on size exclusion column (Superdex 200 HR 10/30, Amersham Biosciences). The buffer used for gel filtration was 50 mM HEPES pH 7.5, 200 mM NaCl, 5 mM Glutathione, 3% Glycerol. Fractions containing c-fms were pooled, passed through a 0.1 μm vacuum filter, incubated with a compound for 2 hours and concentrated to a final concentration of 7 to 11 mg/ml.

### Example 3

### Enzymatic Assays

(a) An autophosphorylation, fluorescence polarization competition immunoassay was used for compounds with IC₅₀'s >10 nM. The assay was performed in black 96-well micro plates (UL BioSystems). The assay buffer used was 100 mM HEPES, pH 7.5, 1 mM DTT, 0.01% (v/v) Tween-20. Compounds were diluted in assay-buffer containing 4% DMSO just prior to the assay. To each well, 5 μl of compound were added followed by the addition of 3 μl of a mix containing 33 nM c-fms (3DP) and 16.7 mM MgCl₂ (Sigma) in assay buffer. The kinase reaction was initiated by adding 2 μl of 5 mM ATP (Sigma) in assay buffer. The final concentrations in the assay were 10 nM c-fms, 1 mM ATP, 5 mM MgCl₂, 2% DMSO. Control reactions were ran in each plate: in positive and negative control wells, assay buffer (made 4% in DMSO) was substituted for the compound; in addition, positive control wells received 1.2 μl of 50 mM EDTA.

The plates were covered and incubated at room temperature for 45 min. At the end of the incubation, the reaction was quenched with 1.2 μl of 50 mM EDTA (EDTA was not added to the positive control wells at this point; see above). Following a 5-min incubation, each well received 10 μl of a 1:1:3 mixture of anti-phosphotyrosine antibody, 10X, PTK green tracer, 10X (vortexed), FP dilution buffer, respectively (all from PanVera, cat. # P2837). The plate was covered, incubated for 30 min at room temperature and the fluorescence polarization was read on the Analyst. The instrument settings were: 485 nm excitation filter; 530 nm emission filter; Z height: middle of well; G factor: 0.93. Under these conditions, the fluorescence polarization values for positive and negative controls were ˜300 and ˜150, respectively, and were used to define the 100% and 0% inhibition of the c-fms reaction. The IC₅₀ values reported are the averages of three independent measurements.

(b) A peptide phosphorylation, fluorescence polarization competition immunoassay was used for compounds with IC₅₀'s <10 nM. The assay was performed in black 96-well micro plates. The assay buffer used was 100 mM HEPES, pH 7.5, 1 mM DTT, 0.01% (v/v) Tween-20. Compounds were diluted in assay buffer containing 4% DMSO just prior to the assay. To each well, 5 μl of compound were added followed by the addition of 2 μl of a mix containing 5 nM c-fms and 25 mM MgCl₂ in assay buffer. 2 μl of 1540 μM peptide SYEGNSYTFIDPTQ (AnaSpec) were subsequently added. The kinase reaction was initiated by adding 1 μl of 10 mM ATP in assay buffer. The final concentrations in the assay were 1 nM c-fms, 308 μM peptide, 1 mM ATP, 5 mM MgCl₂, 2% DMSO. Control reactions were ran in each plate: in positive and negative control wells, assay buffer (made 4% in DMSO) was substituted for the compound; in addition, positive control wells received 1.2 μl of 50 mM EDTA. The plates were covered and incubated at room temperature for 80 min. Quenching of the reaction and detection of product formation were performed as described in (a). Fluorescence polarization values for positive and negative controls were ˜290 and ˜160, respectively, and were used to define the 100% and 0% inhibition of the c-fms reaction.

### Example 4

### Crystallographic Detail

Crystallization and Structure Determination

In a typical crystallization experiment 1-2 μl of c-fms protein complexed with the lead compounds and concentrated to 7-10 mg/ml was mixed in a 1:1 ratio with well solution (15-28% PEG 3350, 100 mM Sodium-Acetate pH 5.0-5.6, 200 mM Li₂SO₄, 5 mM DTT, 0-3% glycerol) and placed on a glass cover slip. The cover slip was inverted and sealed over a reservoir of 500-1000 μl of well solution and incubated at 22° C. Crystals usually appeared over night. In most cases μ-seeding with a seed stock obtained from one of the lead compounds was used to induce crystallization. Crystals were harvested with a nylon loop, placed for less than 10 seconds in cryo-solution (27% PEG 3350, 100 mM. Sodium-Acetate pH 5.5, 200 mM Li₂SO₄, 5 mM DTT, 10% glycerol) and frozen by immersion in liquid nitrogen. Data were collected at 100K on a Bruker AXS MO6XCE rotating anode and a SMART 6000 CCD detector or at the IMCA-CAT ID-17 beamline at the Argonne National Laboratory. The diffraction data was processed with the Bruker Proteum suite or the HKL suite (Denzo/Scalepack) The initial c-fms structure was solved by molecular replacement using the FGFR crystal structure as a search model in CNX. Structure refinement and model building was carried out according to standard protocols using CNX [10] and O [11].

Crystal form I was obtained with the following characteristics: a=81.07, b=81.07, c=144.67, α=90, β=90, γ=120, sg=R3, diffraction limit 1.9 Å (synchrotron), one molecule/ASU. Crystal form II was obtained with the following characteristics: a=53.1, b=72.4, c=91.7, α=90, β=90, γ=90, sg=P2₁2₁2₁, diffraction limit 3 Å (synchrotron), one molecule/asymmetric unit.

A highly preferred crystal structure is a crystal structure defined by structure coordinates of c-fms amino acids Trp 550, Lys 586, Thr 587, Leu 588, Gly 589, Val 596, Glu 598, Ala 614, Val 615, Lys 616, Glu 633, Met 637, Leu 640, Ile 646, Val 647, Val 661, Thr 663, Glu 664, Tyr 665, Cys 666, Cys 667, Tyr 668, Gly 669, Asp 670, Asn 673, Arg 677, Cys 774, Ile 775, His 776, Arg 782, Asn 783, Leu 785, Ile 794, Gly 795, Asp 796, Phe 797, Gly 798, Leu 799, Ala 800, Arg 801, Asp 802.

A preferred crystal structure is a crystal structure defined by structure coordinates of c-fms amino acid residues Lys 586, Leu 588, Gly 589, Val 596, Glu 598, Ala 614, Lys 616, Val 647, Thr 663, Glu 664, Tyr 665, Cys 666, Cys 667, Tyr 668, Gly 669, Asp 670, Arg 677, Arg 782, Leu 785, Asp 796, Phe 797, Gly 798, Leu 799, Ala 800, Arg 801, Asp 802.

Another crystal structure of the invention is defined by structure coordinates of c-fms amino acid residues Lys 586, Leu 588, Gly 589, Val 596, Glu 598, Ala 614, Lys 616, Val 647, Thr 663, Glu 664, Tyr 665, Cys 666, Cys 667, Tyr 668, Gly 669, Asp 670, Arg 782, Asn 783, Leu 785, Asp 796, Phe 797, Leu 799, Ala 800, Arg 801.

Ligands of c-fms

Binding Mode of the Arylamide Series

The inhibitor of the arylamide series occupies the nucleotide-binding pocket, located between the N-domain and the C-domain. The carbonyl oxygen of the amide bond forms a hydrogen bond with the amide-N of Cys666 in the hinge-region. The five-membered ring together with the cyano-group occupies the adenine pocket. A π-π stacking interaction is formed with Phe797 of the DFG motif. Other van der waals interactions are mediated by the surrounding hydrophobic pocket formed by Val596, Ala614, Lys616, Val647, Thr663, Leu785 and Ala800. The ortho-methyl-piperidine ring is located in the sugar pocket. Arg801 forms the bottom of that pocket and Asn783 and Gly589 are flanking the piperidine ring on either side. The methoxy-aryl ring projects into the solvent area and interacts with part of the solvent interface residues, mainly Leu588 and Gly669. A weak hydrogen bonding interaction between methyl-hydroxy group and the phenol-hydroxy group of Tyr665 can be observed as well. A general description of the acrylamide series is set forth, below.

Binding Mode of Quinolone Series

The quinolone also occupies the nucleotide-binding pocket, with the chloro-aryl ring located in the adenine pocket. Both the amide oxygen and nitrogen form hydrogen-bonding interactions with the backbone of the hinge residues Cys666 and Glu664. The remainder of the interactions is mainly of hydrophobic nature and involves residues of the solvent interface and the sugar pocket (Leu588, Gly589, Leu596, Gly669, Asp670, Leu785, Phe797, Ala800, Arg801). A general description of the quinolone series is set forth below.

Specific ligands used in this invention:

TABLE 1
Coordinates of c-FMS (FGF-chimera) in complex
with 1183648 (Arylamide)
1183648 Arylamide Series

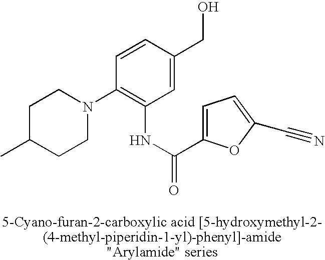

793693 Quinolone Series

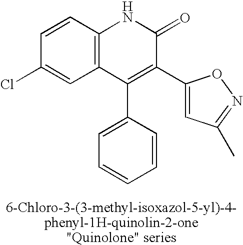

REMARK c-fins (538-922, FGF chimera) complexed with 1183648
REMARK refinement resolution: 500.0-1.9 A
REMARK starting r = 0.2455 free_r = 0.2709
REMARK final r = 0.2382 free_r = 0.2634
CRYST1 81.070 81.070 144.670 90.00 90.00 120.00 R 3
REMARK Written by CMX VERSION: 2000.12

| | | | | | | | | | | | | |
|---|---|---|---|---|---|---|---|---|---|---|---|---|
| ATOM | 1 | GB | VAL | A | 548 | 11.165 | 23.567 | −9.796 | 1.00 | 46.87 | A | C |
| ATOM | 2 | CG1 | VAL | A | 548 | 10.117 | 22.545 | −10.233 | 1.00 | 48.46 | A | C |
| ATOM | 3 | CG2 | VAL | A | 548 | 11.507 | 24.515 | −10.930 | 1.00 | 47.89 | A | C |
| ATOM | 4 | C | VAL | A | 548 | 10.853 | 23.525 | −7.318 | 1.00 | 44.87 | A | C |
| ATOM | 5 | O | VAL | A | 548 | 10.292 | 23.832 | −6.266 | 1.00 | 46.87 | A | O |
| ATOM | 6 | N | VAL | A | 548 | 11.394 | 25.649 | −8.429 | 1.00 | 47.54 | A | N |
| ATOM | 7 | CA | VAL | A | 548 | 10.652 | 24.363 | −8.578 | 1.00 | 46.32 | A | C |
| ATOM | 8 | N | ARG | A | 549 | 11.665 | 22.476 | −7.423 | 1.00 | 42.02 | A | N |
| ATOM | 9 | CA | ARG | A | 549 | 11.938 | 21.604 | −6.285 | 1.00 | 37.85 | A | C |
| ATOM | 10 | CB | ARG | A | 549 | 11.190 | 20.275 | −6.452 | 1.00 | 41.18 | A | C |
| ATOM | 11 | CG | ARG | A | 549 | 9.696 | 20.418 | −6.734 | 1.00 | 46.48 | A | C |
| ATOM | 12 | CD | ARG | A | 549 | 8.961 | 21.106 | −5.591 | 1.00 | 49.39 | A | C |
| ATOM | 13 | NE | ARG | A | 549 | 7.583 | 21.443 | −5.947 | 1.00 | 53.70 | A | N |
| ATOM | 14 | CZ | ARG | A | 549 | 6.645 | 20.553 | −6.263 | 1.00 | 54.90 | A | C |
| ATOM | 15 | NH1 | ARG | A | 549 | 6.924 | 19.256 | −6.273 | 1.00 | 55.44 | A | N |
| ATOM | 16 | NH2 | ARG | A | 549 | 5.421 | 20.961 | −6.565 | 1.00 | 54.85 | A | N |
| ATOM | 17 | C | ARG | A | 549 | 13.435 | 21.329 | −6.157 | 1.00 | 33.97 | A | C |
| ATOM | 18 | O | ARG | A | 549 | 14.173 | 21.380 | −7.145 | 1.00 | 30.81 | A | O |
| ATOM | 19 | N | TRP | A | 550 | 13.880 | 21.052 | −4.933 | 1.00 | 29.62 | A | N |
| ATOM | 20 | CA | TRP | A | 550 | 15.283 | 20.740 | −4.666 | 1.00 | 28.56 | A | C |
| ATOM | 21 | CB | TRP | A | 550 | 15.504 | 20.519 | −3.171 | 1.00 | 26.01 | A | C |
| ATOM | 22 | CG | TRP | A | 550 | 15.550 | 21.786 | −2.386 | 1.00 | 25.25 | A | C |
| ATOM | 23 | CD2 | TRP | A | 550 | 16.091 | 21.955 | −1.074 | 1.00 | 23.72 | A | C |
| ATOM | 24 | CE2 | TRP | A | 550 | 15.908 | 23.307 | −0.717 | 1.00 | 22.59 | A | C |
| ATOM | 25 | CE3 | TRP | A | 550 | 16.713 | 21.090 | −0.163 | 1.00 | 24.79 | A | C |
| ATOM | 26 | CD1 | TRP | A | 550 | 15.069 | 23.012 | −2.768 | 1.00 | 24.22 | A | C |
| ATOM | 27 | NE1 | TRP | A | 550 | 15.283 | 23.930 | −1.765 | 1.00 | 22.76 | A | N |
| ATOM | 28 | CZ2 | TRP | A | 550 | 16.328 | 23.821 | 0.515 | 1.00 | 21.82 | A | C |
| ATOM | 29 | CZ3 | TRP | A | 550 | 17.128 | 21.602 | 1.068 | 1.00 | 24.34 | A | C |
| ATOM | 30 | CH2 | TRP | A | 550 | 16.932 | 22.955 | 1.391 | 1.00 | 22.28 | A | C |
| ATOM | 31 | C | TRP | A | 550 | 15.657 | 19.475 | −5.410 | 1.00 | 29.29 | A | C |
| ATOM | 32 | O | TRP | A | 550 | 14.826 | 18.589 | −5.571 | 1.00 | 28.55 | A | O |
| ATOM | 33 | N | LYS | A | 551 | 16.910 | 19.386 | −5.843 | 1.00 | 30.00 | A | N |
| ATOM | 34 | CA | LYS | A | 551 | 17.365 | 18.224 | −6.590 | 1.00 | 33.37 | A | C |
| ATOM | 35 | CB | LYS | A | 551 | 16.932 | 18.370 | −8.052 | 1.00 | 35.38 | A | C |
| ATOM | 36 | CG | LYS | A | 551 | 17.405 | 17.253 | −8.976 | 1.00 | 39.80 | A | C |
| ATOM | 37 | CD | LYS | A | 551 | 16.825 | 17.431 | −10.367 | 1.00 | 42.63 | A | C |
| ATOM | 38 | CE | LYS | A | 551 | 17.256 | 16.315 | −11.302 | 1.00 | 44.35 | A | C |
| ATOM | 39 | NZ | LYS | A | 551 | 16.549 | 16.416 | −12.605 | 1.00 | 48.76 | A | N |
| ATOM | 40 | C | LYS | A | 551 | 18.875 | 18.013 | −6.523 | 1.00 | 32.42 | A | C |
| ATOM | 41 | O | LYS | A | 551 | 19.655 | 18.962 | −6.622 | 1.00 | 32.55 | A | O |
| ATOM | 42 | N | ILE | A | 552 | 19.282 | 16.760 | −6.352 | 1.00 | 33.95 | A | N |
| ATOM | 43 | CA | ILE | A | 552 | 20.697 | 16.424 | −6.305 | 1.00 | 35.46 | A | C |
| ATOM | 44 | CB | ILE | A | 552 | 20.953 | 15.123 | −5.503 | 1.00 | 35.03 | A | C |
| ATOM | 45 | CG2 | ILE | A | 552 | 22.436 | 14.785 | −5.526 | 1.00 | 34.39 | A | C |
| ATOM | 46 | CG1 | ILE | A | 552 | 20.460 | 15.285 | −4.059 | 1.00 | 35.72 | A | C |
| ATOM | 47 | CD1 | ILE | A | 552 | 21.162 | 16.378 | −3.276 | 1.00 | 34.12 | A | C |
| ATOM | 48 | C | ILE | A | 552 | 21.167 | 16.214 | −7.742 | 1.00 | 37.59 | A | C |
| ATOM | 49 | O | ILE | A | 552 | 20.582 | 15.425 | −8.481 | 1.00 | 37.74 | A | O |
| ATOM | 50 | N | ILE | A | 553 | 22.214 | 16.932 | −8.134 | 1.00 | 39.45 | A | N |
| ATOM | 51 | CA | ILE | A | 553 | 22.763 | 16.820 | −9.479 | 1.00 | 42.83 | A | C |
| ATOM | 52 | CB | ILE | A | 553 | 22.910 | 18.202 | −10.134 | 1.00 | 43.54 | A | C |
| ATOM | 53 | CG2 | ILE | A | 553 | 21.547 | 18.887 | −10.204 | 1.00 | 44.16 | A | C |
| ATOM | 54 | CG1 | ILE | A | 553 | 23.894 | 19.054 | −9.331 | 1.00 | 44.09 | A | C |
| ATOM | 55 | CD1 | ILE | A | 553 | 24.230 | 20.373 | −9.976 | 1.00 | 46.56 | A | C |
| ATOM | 56 | C | ILE | A | 553 | 24.134 | 16.144 | −9.426 | 1.00 | 45.11 | A | C |
| ATOM | 57 | O | ILE | A | 553 | 24.637 | 15.843 | −8.347 | 1.00 | 43.96 | A | O |
| ATOM | 58 | N | GLU | A | 554 | 24.740 | 15.921 | −10.589 | 1.00 | 48.95 | A | N |
| ATOM | 59 | CA | GLU | A | 554 | 26.039 | 15.255 | −10.658 | 1.00 | 53.03 | A | C |
| ATOM | 60 | CB | GLU | A | 554 | 26.065 | 14.318 | −11.868 | 1.00 | 55.27 | A | C |
| ATOM | 61 | CG | GLU | A | 554 | 24.945 | 13.294 | −11.868 | 1.00 | 57.79 | A | C |
| ATOM | 62 | CD | GLU | A | 554 | 25.028 | 12.331 | −13.036 | 1.00 | 59.93 | A | C |
| ATOM | 63 | OE1 | GLU | A | 554 | 25.019 | 12.793 | −14.198 | 1.00 | 61.39 | A | O |
| ATOM | 64 | OE2 | GLU | A | 554 | 25.098 | 11.108 | −12.790 | 1.00 | 61.06 | A | O |
| ATOM | 65 | C | GLU | A | 554 | 27.267 | 16.169 | −10.697 | 1.00 | 55.01 | A | C |
| ATOM | 66 | O | GLU | A | 554 | 27.377 | 17.039 | −11.558 | 1.00 | 55.58 | A | O |
| ATOM | 67 | N | SER | A | 555 | 28.182 | 15.943 | −9.754 | 1.00 | 57.81 | A | N |
| ATOM | 68 | CA | SER | A | 555 | 29.443 | 16.683 | −9.615 | 1.00 | 61.03 | A | C |
| ATOM | 69 | GB | SER | A | 555 | 30.467 | 16.151 | −10.620 | 1.00 | 61.30 | A | C |
| ATOM | 70 | OG | SER | A | 555 | 31.014 | 14.925 | −10.176 | 1.00 | 63.33 | A | O |
| ATOM | 71 | C | SER | A | 555 | 29.456 | 18.210 | −9.673 | 1.00 | 62.83 | A | C |
| ATOM | 72 | O | SER | A | 555 | 28.832 | 18.826 | −10.539 | 1.00 | 64.30 | A | O |
| ATOM | 73 | N | TYR | A | 556 | 30.201 | 18.804 | −8.741 | 1.00 | 64.60 | A | N |
| ATOM | 74 | CA | TYR | A | 556 | 30.358 | 20.255 | −8.646 | 1.00 | 66.35 | A | C |
| ATOM | 75 | CB | TYR | A | 556 | 29.013 | 20.938 | −8.343 | 1.00 | 67.32 | A | C |
| ATOM | 76 | CG | TYR | A | 556 | 29.133 | 22.442 | −8.231 | 1.00 | 68.68 | A | C |
| ATOM | 77 | CD1 | TYR | A | 556 | 29.739 | 23.185 | −9.245 | 1.00 | 69.26 | A | C |
| ATOM | 78 | CE1 | TYR | A | 556 | 29.938 | 24.555 | −9.113 | 1.00 | 69.44 | A | C |
| ATOM | 79 | CD2 | TYR | A | 556 | 28.719 | 23.114 | −7.078 | 1.00 | 68.99 | A | C |
| ATOM | 80 | CE2 | TYR | A | 556 | 28.914 | 24.489 | −6.938 | 1.00 | 69.55 | A | C |
| ATOM | 81 | CZ | TYR | A | 556 | 29.524 | 25.201 | −7.960 | 1.00 | 69.54 | A | C |
| ATOM | 82 | OH | TYR | A | 556 | 29.723 | 26.557 | −7.833 | 1.00 | 70.57 | A | O |
| ATOM | 83 | C | TYR | A | 556 | 31.407 | 20.656 | −7.593 | 1.00 | 66.71 | A | C |
| ATOM | 84 | O | TYR | A | 556 | 32.451 | 20.010 | −7.474 | 1.00 | 66.92 | A | O |
| ATOM | 85 | N | GLU | A | 557 | 31.121 | 21.716 | −6.837 | 1.00 | 66.87 | A | N |
| ATOM | 86 | CA | GLU | A | 557 | 32.017 | 22.249 | −5.814 | 1.00 | 67.02 | A | C |
| ATOM | 87 | CB | GLU | A | 557 | 31.976 | 21.382 | −4.553 | 1.00 | 66.49 | A | C |
| ATOM | 88 | CG | GLU | A | 557 | 30.717 | 21.567 | −3.721 | 1.00 | 65.18 | A | C |
| ATOM | 89 | CD | GLU | A | 557 | 30.665 | 22.918 | −3.023 | 1.00 | 64.82 | A | C |
| ATOM | 90 | OE1 | GLU | A | 557 | 31.463 | 23.140 | −2.086 | 1.00 | 62.98 | A | O |
| ATOM | 91 | OE2 | GLU | A | 557 | 29.826 | 23.758 | −3.415 | 1.00 | 64.85 | A | O |
| ATOM | 92 | C | GLU | A | 557 | 33.449 | 22.369 | −6.317 | 1.00 | 67.59 | A | C |
| ATOM | 93 | O | GLU | A | 557 | 33.798 | 23.344 | −6.986 | 1.00 | 68.31 | A | O |
| ATOM | 94 | N | SER | A | 560 | 31.023 | 18.654 | −4.540 | 1.00 | 49.29 | A | N |
| ATOM | 95 | CA | SER | A | 560 | 31.861 | 17.507 | −4.211 | 1.00 | 47.58 | A | C |
| ATOM | 96 | GB | SER | A | 560 | 31.617 | 17.077 | −2.765 | 1.00 | 49.09 | A | C |
| ATOM | 97 | OG | SER | A | 560 | 32.464 | 15.999 | −2.405 | 1.00 | 52.73 | A | O |
| ATOM | 98 | C | SER | A | 560 | 31.543 | 16.355 | −5.160 | 1.00 | 45.63 | A | C |
| ATOM | 99 | O | SER | A | 560 | 31.309 | 16.574 | −6.348 | 1.00 | 46.90 | A | O |
| ATOM | 100 | N | TYR | A | 561 | 31.526 | 15.132 | −4.640 | 1.00 | 43.44 | A | N |
| ATOM | 101 | CA | TYR | A | 561 | 31.237 | 13.972 | −5.477 | 1.00 | 40.83 | A | C |
| ATOM | 102 | CB | TYR | A | 561 | 32.342 | 12.921 | −5.336 | 1.00 | 38.21 | A | C |
| ATOM | 103 | CG | TYR | A | 561 | 32.374 | 11.927 | −6.474 | 1.00 | 36.42 | A | C |
| ATOM | 104 | CD1 | TYR | A | 561 | 33.156 | 12.161 | −7.610 | 1.00 | 35.20 | A | C |
| ATOM | 105 | CE1 | TYR | A | 561 | 33.169 | 11.259 | −8.670 | 1.00 | 33.53 | A | C |
| ATOM | 106 | CD2 | TYR | A | 561 | 31.605 | 10.767 | −6.432 | 1.00 | 34.66 | A | C |
| ATOM | 107 | CE2 | TYR | A | 561 | 31.607 | 9.863 | −7.486 | 1.00 | 32.90 | A | C |
| ATOM | 108 | CZ | TYR | A | 561 | 32.390 | 10.113 | −8.599 | 1.00 | 31.59 | A | C |
| ATOM | 109 | OH | TYR | A | 561 | 32.402 | 9.215 | −9.635 | 1.00 | 34.61 | A | O |
| ATOM | 110 | C | TYR | A | 561 | 29.896 | 13.351 | −5.103 | 1.00 | 40.32 | A | C |
| ATOM | 111 | O | TYR | A | 561 | 29.597 | 13.156 | −3.924 | 1.00 | 41.32 | A | O |
| ATOM | 112 | N | THR | A | 562 | 29.088 | 13.054 | −6.114 | 1.00 | 38.96 | A | N |
| ATOM | 113 | CA | THR | A | 562 | 27.782 | 12.442 | −5.900 | 1.00 | 39.31 | A | C |
| ATOM | 114 | CB | THR | A | 562 | 26.709 | 13.046 | −6.841 | 1.00 | 40.36 | A | C |
| ATOM | 115 | OG1 | THR | A | 562 | 26.538 | 14.441 | −6.552 | 1.00 | 41.37 | A | O |
| ATOM | 116 | CG2 | THR | A | 562 | 25.375 | 12.329 | −6.658 | 1.00 | 41.13 | A | C |
| ATOM | 117 | C | THR | A | 562 | 27.876 | 10.940 | −6.167 | 1.00 | 38.15 | A | C |
| ATOM | 118 | O | THR | A | 562 | 27.992 | 10.520 | −7.310 | 1.00 | 36.90 | A | O |
| ATOM | 119 | N | PHE | A | 563 | 27.828 | 10.138 | −5.108 | 1.00 | 38.25 | A | N |
| ATOM | 120 | CA | PHE | A | 563 | 27.904 | 8.686 | −5.246 | 1.00 | 38.94 | A | C |
| ATOM | 121 | CB | PHE | A | 563 | 28.561 | 8.059 | −4.014 | 1.00 | 37.40 | A | C |
| ATOM | 122 | CG | PHE | A | 563 | 30.024 | 8.376 | −3.874 | 1.00 | 37.56 | A | C |
| ATOM | 123 | CD1 | PHE | A | 563 | 30.444 | 9.509 | −3.188 | 1.00 | 36.54 | A | C |
| ATOM | 124 | CD2 | PHE | A | 563 | 30.982 | 7.537 | −4.439 | 1.00 | 37.87 | A | C |
| ATOM | 125 | CE1 | PHE | A | 563 | 31.803 | 9.805 | −3.062 | 1.00 | 37.79 | A | C |
| ATOM | 126 | CE2 | PHE | A | 563 | 32.342 | 7.823 | −4.322 | 1.00 | 36.75 | A | C |
| ATOM | 127 | CZ | PHE | A | 563 | 32.753 | 8.958 | −3.632 | 1.00 | 37.32 | A | C |
| ATOM | 128 | C | PHE | A | 563 | 26.528 | 8.063 | −5.447 | 1.00 | 39.38 | A | C |
| ATOM | 129 | O | PHE | A | 563 | 26.370 | 7.111 | −6.219 | 1.00 | 39.09 | A | O |
| ATOM | 130 | N | ILE | A | 564 | 25.539 | 8.601 | −4.742 | 1.00 | 40.69 | A | N |
| ATOM | 131 | CA | ILE | A | 564 | 24.170 | 8.112 | −4.831 | 1.00 | 41.77 | A | C |
| ATOM | 132 | CB | ILE | A | 564 | 23.702 | 7.503 | −3.490 | 1.00 | 42.05 | A | C |
| ATOM | 133 | CG2 | ILE | A | 564 | 24.395 | 6.184 | −3.250 | 1.00 | 43.03 | A | C |
| ATOM | 134 | CG1 | ILE | A | 564 | 23.986 | 8.477 | −2.344 | 1.00 | 42.97 | A | C |
| ATOM | 135 | CD1 | ILE | A | 564 | 23.578 | 7.955 | −0.974 | 1.00 | 43.33 | A | C |
| ATOM | 136 | C | ILE | A | 564 | 23.231 | 9.254 | −5.205 | 1.00 | 43.18 | A | C |
| ATOM | 137 | O | ILE | A | 564 | 23.075 | 9.570 | −6.385 | 1.00 | 44.14 | A | O |
| ATOM | 138 | N | GLU | A | 573 | 14.255 | 3.524 | 7.607 | 1.00 | 51.06 | A | N |
| ATOM | 139 | CA | GLU | A | 573 | 12.990 | 4.014 | 8.131 | 1.00 | 48.88 | A | C |
| ATOM | 140 | CB | GLU | A | 573 | 11.937 | 2.909 | 8.066 | 1.00 | 50.91 | A | C |
| ATOM | 141 | CG | GLU | A | 573 | 10.638 | 3.365 | 7.428 | 1.00 | 54.45 | A | C |
| ATOM | 142 | CD | GLU | A | 573 | 9.700 | 2.220 | 7.137 | 1.00 | 54.34 | A | C |
| ATOM | 143 | OE1 | GLU | A | 573 | 9.200 | 1.602 | 8.101 | 1.00 | 57.46 | A | O |
| ATOM | 144 | OE2 | GLU | A | 573 | 9.469 | 1.936 | 5.944 | 1.00 | 54.86 | A | O |
| ATOM | 145 | C | GLU | A | 573 | 13.158 | 4.505 | 9.566 | 1.00 | 47.33 | A | C |
| ATOM | 146 | O | GLU | A | 573 | 12.230 | 4.448 | 10.378 | 1.00 | 45.02 | A | O |
| ATOM | 147 | N | LYS | A | 574 | 14.358 | 4.992 | 9.862 | 1.00 | 45.22 | A | N |
| ATOM | 148 | CA | LYS | A | 574 | 14.683 | 5.512 | 11.181 | 1.00 | 43.34 | A | C |
| ATOM | 149 | CB | LYS | A | 574 | 16.175 | 5.839 | 11.256 | 1.00 | 45.54 | A | C |
| ATOM | 150 | CG | LYS | A | 574 | 16.626 | 6.882 | 10.231 | 1.00 | 48.43 | A | C |
| ATOM | 151 | CD | LYS | A | 574 | 18.138 | 7.079 | 10.253 | 1.00 | 50.08 | A | C |
| ATOM | 152 | CE | LYS | A | 574 | 18.584 | 8.056 | 9.177 | 1.00 | 50.70 | A | C |
| ATOM | 153 | NZ | LYS | A | 574 | 20.071 | 8.155 | 9.084 | 1.00 | 52.42 | A | N |
| ATOM | 154 | C | LYS | A | 574 | 13.872 | 6.775 | 11.436 | 1.00 | 40.84 | A | C |
| ATOM | 155 | O | LYS | A | 574 | 13.698 | 7.192 | 12.581 | 1.00 | 38.62 | A | O |
| ATOM | 156 | N | TRP | A | 575 | 13.378 | 7.379 | 10.358 | 1.00 | 37.54 | A | N |
| ATOM | 157 | CA | TRP | A | 575 | 12.597 | 8.607 | 10.458 | 1.00 | 35.85 | A | C |
| ATOM | 158 | CB | TRP | A | 575 | 12.668 | 9.390 | 9.142 | 1.00 | 35.33 | A | C |
| ATOM | 159 | CG | TRP | A | 575 | 14.044 | 9.829 | 8.789 | 1.00 | 33.45 | A | C |
| ATOM | 160 | CD2 | TRP | A | 575 | 14.756 | 10.924 | 9.364 | 1.00 | 33.42 | A | C |
| ATOM | 161 | CE2 | TRP | A | 575 | 16.034 | 10.955 | 8.760 | 1.00 | 33.47 | A | C |
| ATOM | 162 | CE3 | TRP | A | 575 | 14.440 | 11.884 | 10.334 | 1.00 | 33.14 | A | C |
| ATOM | 163 | CD1 | TRP | A | 575 | 14.890 | 9.252 | 7.884 | 1.00 | 34.38 | A | C |
| ATOM | 164 | NE1 | TRP | A | 575 | 16.089 | 9.924 | 7.861 | 1.00 | 33.20 | A | N |
| ATOM | 165 | CZ2 | TRP | A | 575 | 16.996 | 11.911 | 9.095 | 1.00 | 34.42 | A | C |
| ATOM | 166 | CZ3 | TRP | A | 575 | 15.397 | 12.834 | 10.667 | 1.00 | 33.68 | A | C |
| ATOM | 167 | CH2 | TRP | A | 575 | 16.660 | 12.840 | 10.047 | 1.00 | 33.09 | A | C |
| ATOM | 168 | C | TRP | A | 575 | 11.135 | 8.392 | 10.816 | 1.00 | 34.58 | A | C |
| ATOM | 169 | O | TRP | A | 575 | 10.419 | 9.352 | 11.078 | 1.00 | 35.64 | A | O |
| ATOM | 170 | N | GLU | A | 576 | 10.688 | 7.140 | 10.830 | 1.00 | 33.31 | A | N |
| ATOM | 171 | CA | GLU | A | 576 | 9.291 | 6.848 | 11.129 | 1.00 | 34.26 | A | C |
| ATOM | 172 | CB | GLU | A | 576 | 9.036 | 5.341 | 11.030 | 1.00 | 33.19 | A | C |
| ATOM | 173 | CG | GLU | A | 576 | 7.577 | 4.961 | 10.804 | 1.00 | 35.33 | A | C |
| ATOM | 174 | CD | GLU | A | 576 | 6.969 | 5.627 | 9.571 | 1.00 | 36.95 | A | C |
| ATOM | 175 | OE1 | GLU | A | 576 | 7.710 | 5.905 | 8.606 | 1.00 | 34.78 | A | O |
| ATOM | 176 | OE2 | GLU | A | 576 | 5.739 | 5.855 | 9.566 | 1.00 | 37.14 | A | O |
| ATOM | 177 | C | GLU | A | 576 | 8.859 | 7.364 | 12.496 | 1.00 | 34.78 | A | C |
| ATOM | 178 | O | GLU | A | 576 | 9.556 | 7.177 | 13.493 | 1.00 | 34.79 | A | O |
| ATOM | 179 | N | PHE | A | 577 | 7.704 | 8.025 | 12.522 | 1.00 | 34.65 | A | N |
| ATOM | 180 | CA | PHE | A | 577 | 7.137 | 8.587 | 13.747 | 1.00 | 35.89 | A | C |
| ATOM | 181 | CB | PHE | A | 577 | 7.324 | 10.112 | 13.743 | 1.00 | 35.50 | A | C |
| ATOM | 182 | CG | PHE | A | 577 | 6.915 | 10.786 | 15.026 | 1.00 | 38.52 | A | C |
| ATOM | 183 | CD1 | PHE | A | 577 | 7.692 | 10.660 | 16.173 | 1.00 | 39.23 | A | C |
| ATOM | 184 | CD2 | PHE | A | 577 | 5.754 | 11.551 | 15.086 | 1.00 | 39.04 | A | C |
| ATOM | 185 | CE1 | PHE | A | 577 | 7.319 | 11.288 | 17.359 | 1.00 | 40.42 | A | C |
| ATOM | 186 | CE2 | PHE | A | 577 | 5.371 | 12.183 | 16.269 | 1.00 | 40.12 | A | C |
| ATOM | 187 | CZ | PHE | A | 577 | 6.157 | 12.052 | 17.405 | 1.00 | 39.09 | A | C |
| ATOM | 188 | C | PHE | A | 577 | 5.646 | 8.232 | 13.775 | 1.00 | 35.65 | A | C |
| ATOM | 189 | O | PHE | A | 577 | 5.001 | 8.178 | 12.729 | 1.00 | 34.99 | A | O |
| ATOM | 190 | N | PRO | A | 578 | 5.082 | 7.966 | 14.969 | 1.00 | 37.40 | A | N |
| ATOM | 191 | CD | PRO | A | 578 | 5.754 | 7.831 | 16.272 | 1.00 | 37.91 | A | C |
| ATOM | 192 | CA | PRO | A | 578 | 3.659 | 7.620 | 15.082 | 1.00 | 39.09 | A | C |
| ATOM | 193 | CB | PRO | A | 578 | 3.517 | 7.172 | 16.540 | 1.00 | 39.23 | A | C |
| ATOM | 194 | CG | PRO | A | 578 | 4.919 | 6.779 | 16.939 | 1.00 | 39.86 | A | C |
| ATOM | 195 | C | PRO | A | 578 | 2.776 | 8.828 | 14.781 | 1.00 | 39.55 | A | C |
| ATOM | 196 | O | PRO | A | 578 | 2.877 | 9.854 | 15.454 | 1.00 | 39.44 | A | O |
| ATOM | 197 | N | ARG | A | 579 | 1.906 | 8.716 | 13.783 | 1.00 | 40.97 | A | N |
| ATOM | 198 | CA | ARG | A | 579 | 1.044 | 9.842 | 13.441 | 1.00 | 43.14 | A | C |
| ATOM | 199 | CB | ARG | A | 579 | 0.279 | 9.569 | 12.142 | 1.00 | 44.41 | A | C |
| ATOM | 200 | CG | ARG | A | 579 | 0.770 | 8.473 | 12.218 | 1.00 | 43.56 | A | C |
| ATOM | 201 | CD | ARG | A | 579 | −1.421 | 8.287 | 10.854 | 1.00 | 43.26 | A | C |
| ATOM | 202 | NE | AEG | A | 579 | −0.452 | 7.843 | 9.854 | 1.00 | 41.26 | A | N |
| ATOM | 203 | CZ | ARG | A | 579 | −0.279 | 8.419 | 8.668 | 1.00 | 40.59 | A | C |
| ATOM | 204 | NH1 | ARG | A | 579 | −1.009 | 9.472 | 8.322 | 1.00 | 38.73 | A | N |
| ATOM | 205 | NH2 | ARG | A | 579 | 0.630 | 7.944 | 7.828 | 1.00 | 38.95 | A | N |
| ATOM | 206 | C | ARG | A | 579 | 0.066 | 10.194 | 14.558 | 1.00 | 44.85 | A | C |
| ATOM | 207 | O | ARG | A | 579 | −0.367 | 11.339 | 14.668 | 1.00 | 44.89 | A | O |
| ATOM | 208 | N | ASN | A | 580 | −0.282 | 9.216 | 15.390 | 1.00 | 46.52 | A | N |
| ATOM | 209 | CA | ASN | A | 580 | −1.210 | 9.478 | 16.485 | 1.00 | 47.33 | A | C |
| ATOM | 210 | CB | ASN | A | 580 | −1.707 | 8.161 | 17.098 | 1.00 | 49.83 | A | C |
| ATOM | 211 | CG | ASN | A | 580 | −0.578 | 7.283 | 17.597 | 1.00 | 51.93 | A | C |
| ATOM | 212 | OD1 | ASN | A | 580 | 0.141 | 7.644 | 18.527 | 1.00 | 52.98 | A | O |
| ATOM | 213 | ND2 | ASN | A | 580 | −0.417 | 6.119 | 16.974 | 1.00 | 54.36 | A | N |
| ATOM | 214 | C | ASN | A | 580 | −0.545 | 10.350 | 17.548 | 1.00 | 47.28 | A | C |
| ATOM | 215 | O | ASN | A | 580 | −1.215 | 10.901 | 18.425 | 1.00 | 47.53 | A | O |
| ATOM | 216 | N | ASN | A | 581 | 0.776 | 10.474 | 17.457 | 1.00 | 46.66 | A | N |
| ATOM | 217 | CA | ASN | A | 581 | 1.547 | 11.298 | 18.385 | 1.00 | 46.34 | A | C |
| ATOM | 218 | CB | ASN | A | 581 | 2.928 | 10.684 | 18.622 | 1.00 | 46.38 | A | C |
| ATOM | 219 | CG | ASN | A | 581 | 2.909 | 9.601 | 19.680 | 1.00 | 48.64 | A | C |
| ATOM | 220 | OD1 | ASN | A | 581 | 1.958 | 8.828 | 19.777 | 1.00 | 49.08 | A | O |
| ATOM | 221 | ND2 | ASN | A | 581 | 3.967 | 9.535 | 20.476 | 1.00 | 49.59 | A | N |
| ATOM | 222 | C | ASN | A | 581 | 1.697 | 12.696 | 17.799 | 1.00 | 45.48 | A | C |
| ATOM | 223 | O | ASN | A | 581 | 2.598 | 13.448 | 18.163 | 1.00 | 45.18 | A | O |
| ATOM | 224 | N | LEU | A | 582 | 0.790 | 13.030 | 16.889 | 1.00 | 46.64 | A | N |
| ATOM | 225 | CA | LEU | A | 582 | 0.788 | 14.320 | 16.213 | 1.00 | 47.46 | A | C |
| ATOM | 226 | CB | LEU | A | 582 | 1.021 | 14.099 | 14.716 | 1.00 | 47.46 | A | C |
| ATOM | 227 | CG | LEU | A | 582 | 2.089 | 14.897 | 13.968 | 1.00 | 47.95 | A | C |
| ATOM | 228 | CD1 | LEU | A | 582 | 3.427 | 14.798 | 14.684 | 1.00 | 47.18 | A | C |
| ATOM | 229 | CD2 | LEU | A | 582 | 2.196 | 14.353 | 12.553 | 1.00 | 46.30 | A | C |
| ATOM | 230 | C | LEU | A | 582 | −0.552 | 15.027 | 16.417 | 1.00 | 47.70 | A | C |
| ATOM | 231 | O | LEU | A | 582 | −1.611 | 14.442 | 16.181 | 1.00 | 48.34 | A | O |
| ATOM | 232 | N | GLN | A | 583 | −0.509 | 16.279 | 16.863 | 1.00 | 47.09 | A | N |
| ATOM | 233 | CA | GLN | A | 583 | −1.730 | 17.050 | 17.061 | 1.00 | 47.88 | A | C |
| ATOM | 234 | CB | GLN | A | 583 | −1.826 | 17.565 | 18.501 | 1.00 | 50.09 | A | C |
| ATOM | 235 | CG | GLN | A | 583 | −3.178 | 18.186 | 18.834 | 1.00 | 52.33 | A | C |
| ATOM | 236 | CD | GLN | A | 583 | −3.248 | 18.738 | 20.246 | 1.00 | 54.36 | A | C |
| ATOM | 237 | OE1 | GLN | A | 583 | −4.308 | 19.165 | 20.702 | 1.00 | 57.10 | A | O |
| ATOM | 238 | NE2 | GLN | A | 583 | −2.119 | 18.738 | 20.943 | 1.00 | 55.40 | A | N |
| ATOM | 239 | C | GLN | A | 583 | −1.694 | 18.222 | 16.089 | 1.00 | 47.05 | A | C |
| ATOM | 240 | O | GLN | A | 583 | −0.955 | 19.183 | 16.289 | 1.00 | 46.70 | A | O |
| ATOM | 241 | N | PHE | A | 584 | −2.495 | 18.131 | 15.035 | 1.00 | 47.31 | A | N |
| ATOM | 242 | CA | PHE | A | 584 | −2.543 | 19.160 | 14.006 | 1.00 | 47.73 | A | C |
| ATOM | 243 | CB | PHE | A | 584 | −3.227 | 18.603 | 12.757 | 1.00 | 50.38 | A | C |
| ATOM | 244 | CG | PHE | A | 584 | −2.416 | 17.561 | 12.039 | 1.00 | 53.03 | A | C |
| ATOM | 245 | CD1 | PHE | A | 584 | −1.697 | 16.604 | 12.752 | 1.00 | 55.45 | A | C |
| ATOM | 246 | CD2 | PHE | A | 584 | −2.389 | 17.518 | 10.652 | 1.00 | 53.61 | A | C |
| ATOM | 247 | CE1 | PHE | A | 584 | −0.965 | 15.621 | 12.094 | 1.00 | 54.55 | A | C |
| ATOM | 248 | CE2 | PHE | A | 584 | −1.660 | 16.538 | 9.988 | 1.00 | 54.81 | A | C |
| ATOM | 249 | CZ | PHE | A | 584 | −0.948 | 15.587 | 10.711 | 1.00 | 54.82 | A | C |
| ATOM | 250 | C | PHE | A | 584 | −3.204 | 20.470 | 14.412 | 1.00 | 46.10 | A | C |
| ATOM | 251 | O | PHE | A | 584 | −4.168 | 20.496 | 15.181 | 1.00 | 45.13 | A | O |
| ATOM | 252 | N | GLY | A | 585 | −2.661 | 21.558 | 13.872 | 1.00 | 44.07 | A | N |
| ATOM | 253 | CA | GLY | A | 585 | −3.173 | 22.883 | 14.151 | 1.00 | 41.49 | A | C |
| ATOM | 254 | C | GLY | A | 585 | −3.616 | 23.565 | 12.874 | 1.00 | 40.71 | A | C |
| ATOM | 255 | O | GLY | A | 585 | −4.151 | 22.921 | 11.970 | 1.00 | 40.08 | A | O |
| ATOM | 256 | N | LYS | A | 586 | −3.381 | 24.870 | 12.788 | 1.00 | 40.07 | A | N |
| ATOM | 257 | CA | LYS | A | 586 | −3.777 | 25.638 | 11.613 | 1.00 | 40.54 | A | C |
| ATOM | 258 | CB | LYS | A | 586 | −3.736 | 27.137 | 11.917 | 1.00 | 41.93 | A | C |
| ATOM | 259 | CG | LYS | A | 586 | −2.326 | 27.697 | 12.074 | 1.00 | 45.59 | A | C |
| ATOM | 260 | CD | LYS | A | 586 | −2.339 | 29.221 | 12.134 | 1.00 | 47.83 | A | C |
| ATOM | 261 | CE | LYS | A | 586 | −0.933 | 29.794 | 12.215 | 1.00 | 50.42 | A | C |
| ATOM | 262 | NZ | LYS | A | 586 | −0.241 | 29.413 | 13.477 | 1.00 | 50.82 | A | N |
| ATOM | 263 | C | LYS | A | 586 | −2.904 | 25.372 | 10.393 | 1.00 | 38.79 | A | C |
| ATOM | 264 | O | LYS | A | 586 | −1.740 | 24.986 | 10.510 | 1.00 | 37.58 | A | O |
| ATOM | 265 | N | THR | A | 587 | −3.486 | 25.586 | 9.219 | 1.00 | 38.30 | A | N |
| ATOM | 266 | CA | THR | A | 587 | −2.777 | 25.417 | 7.964 | 1.00 | 37.40 | A | C |
| ATOM | 267 | CB | THR | A | 587 | −3.762 | 25.253 | 6.797 | 1.00 | 38.08 | A | C |
| ATOM | 268 | OG1 | THR | A | 587 | −4.560 | 24.082 | 7.014 | 1.00 | 36.18 | A | O |
| ATOM | 269 | CG2 | THR | A | 587 | −3.010 | 25.123 | 5.473 | 1.00 | 37.14 | A | C |
| ATOM | 270 | C | THR | A | 587 | −1.942 | 26.680 | 7.754 | 1.00 | 36.74 | A | C |
| ATOM | 271 | O | THR | A | 587 | −2.435 | 27.788 | 7.939 | 1.00 | 36.53 | A | O |
| ATOM | 272 | N | LEU | A | 588 | −0.678 | 26.506 | 7.378 | 1.00 | 36.11 | A | N |
| ATOM | 273 | CA | LEU | A | 588 | 0.233 | 27.630 | 7.160 | 1.00 | 35.14 | A | C |
| ATOM | 274 | CB | LEU | A | 588 | 1.650 | 27.218 | 7.540 | 1.00 | 35.05 | A | C |
| ATOM | 275 | CG | LEU | A | 588 | 1.792 | 26.730 | 8.984 | 1.00 | 35.11 | A | C |
| ATOM | 276 | CD1 | LEU | A | 588 | 3.142 | 26.057 | 9.173 | 1.00 | 32.99 | A | C |
| ATOM | 277 | CD2 | LEU | A | 588 | 1.625 | 27.910 | 9.944 | 1.00 | 36.00 | A | C |
| ATOM | 278 | C | LEU | A | 588 | 0.211 | 28.110 | 5.717 | 1.00 | 35.47 | A | C |
| ATOM | 279 | O | LEU | A | 588 | 0.338 | 29.306 | 5.443 | 1.00 | 36.82 | A | O |
| ATOM | 280 | N | GLY | A | 589 | 0.050 | 27.168 | 4.798 | 1.00 | 34.05 | A | N |
| ATOM | 281 | CA | GLY | A | 589 | 0.009 | 27.503 | 3.392 | 1.00 | 33.07 | A | C |
| ATOM | 282 | C | GLY | A | 589 | −0.331 | 26.262 | 2.599 | 1.00 | 34.05 | A | C |
| ATOM | 283 | O | GLY | A | 589 | −0.278 | 25.147 | 3.125 | 1.00 | 35.75 | A | O |
| ATOM | 284 | N | ALA | A | 590 | −0.677 | 26.448 | 1.334 | 1.00 | 34.26 | A | N |
| ATOM | 285 | CA | ALA | A | 590 | −1.025 | 25.327 | 0.480 | 1.00 | 35.08 | A | C |
| ATOM | 286 | CB | ALA | A | 590 | −2.458 | 24.881 | 0.760 | 1.00 | 35.99 | A | C |
| ATOM | 287 | C | ALA | A | 590 | −0.876 | 25.694 | −0.983 | 1.00 | 37.94 | A | C |
| ATOM | 288 | O | ALA | A | 590 | −0.955 | 26.867 | −1.353 | 1.00 | 38.44 | A | O |
| ATOM | 289 | N | GLY | A | 591 | −0.652 | 24.674 | −1.806 | 1.00 | 39.30 | A | N |
| ATOM | 290 | CA | GLY | A | 591 | −0.508 | 24.870 | −3.234 | 1.00 | 40.01 | A | C |
| ATOM | 291 | C | GLY | A | 591 | −1.530 | 24.016 | −3.955 | 1.00 | 39.30 | A | C |
| ATOM | 292 | O | GLY | A | 591 | −2.463 | 23.506 | −3.338 | 1.00 | 39.01 | A | O |
| ATOM | 293 | N | ALA | A | 592 | −1.344 | 23.846 | −5.257 | 1.00 | 41.36 | A | N |
| ATOM | 294 | CA | ALA | A | 592 | −2.261 | 23.061 | −6.070 | 1.00 | 42.29 | A | C |
| ATOM | 295 | CB | ALA | A | 592 | −1.834 | 23.124 | −7.526 | 1.00 | 40.91 | A | C |
| ATOM | 296 | C | ALA | A | 592 | −2.381 | 21.603 | −5.633 | 1.00 | 44.01 | A | C |
| ATOM | 297 | O | ALA | A | 592 | −3.470 | 21.033 | −5.654 | 1.00 | 44.46 | A | O |
| ATOM | 298 | N | PHE | A | 593 | −1.275 | 21.002 | −5.217 | 1.00 | 45.09 | A | N |
| ATOM | 299 | CA | PHE | A | 593 | −1.322 | 19.599 | −4.830 | 1.00 | 46.88 | A | C |
| ATOM | 300 | CB | PHE | A | 593 | −0.619 | 18.771 | −5.907 | 1.00 | 49.57 | A | C |
| ATOM | 301 | CG | PHE | A | 593 | −1.259 | 18.887 | −7.263 | 1.00 | 52.95 | A | C |
| ATOM | 302 | CD1 | PHE | A | 593 | −0.491 | 19.157 | −81.393 | 1.00 | 53.94 | A | C |
| ATOM | 303 | CD2 | PHE | A | 593 | −2.633 | 18.725 | −7.412 | 1.00 | 54.24 | A | C |
| ATOM | 304 | CE1 | PHE | A | 593 | −1.082 | 19.265 | −9.651 | 1.00 | 54.02 | A | C |
| ATOM | 305 | CE2 | PHE | A | 593 | −3.234 | 18.832 | −81.664 | 1.00 | 55.12 | A | C |
| ATOM | 306 | CZ | PHE | A | 593 | −2.456 | 19.102 | −9.786 | 1.00 | 55.59 | A | C |
| ATOM | 307 | C | PHE | A | 593 | −0.782 | 19.235 | −3.451 | 1.00 | 45.57 | A | C |
| ATOM | 308 | O | PHE | A | 593 | −0.587 | 18.057 | −3.152 | 1.00 | 46.97 | A | O |
| ATOM | 309 | N | GLY | A | 594 | −0.560 | 20.232 | −2.602 | 1.00 | 43.26 | A | N |
| ATOM | 310 | CA | GLY | A | 594 | −0.061 | 19.944 | −1.271 | 1.00 | 40.32 | A | C |
| ATOM | 311 | C | GLY | A | 594 | −0.270 | 21.087 | −0.301 | 1.00 | 38.53 | A | C |
| ATOM | 312 | O | GLY | A | 594 | −0.659 | 22.186 | −0.698 | 1.00 | 37.40 | A | O |
| ATOM | 313 | N | LYS | A | 595 | −0.015 | 20.831 | 0.977 | 1.00 | 35.98 | A | N |
| ATOM | 314 | CA | LYS | A | 595 | −0.169 | 21.858 | 1.998 | 1.00 | 35.80 | A | C |
| ATOM | 315 | CB | LYS | A | 595 | −1.641 | 21.981 | 2.413 | 1.00 | 39.51 | A | C |
| ATOM | 316 | CG | LYS | A | 595 | −2.189 | 20.797 | 3.197 | 1.00 | 40.62 | A | C |
| ATOM | 317 | CD | LYS | A | 595 | −3.701 | 20.919 | 3.360 | 1.00 | 44.05 | A | C |
| ATOM | 318 | CE | LYS | A | 595 | −4.264 | 19.834 | 4.266 | 1.00 | 46.20 | A | C |
| ATOM | 319 | NZ | LYS | A | 595 | −3.826 | 20.010 | 5.678 | 1.00 | 48.21 | A | N |
| ATOM | 320 | C | LYS | A | 595 | 0.681 | 21.540 | 3.215 | 1.00 | 34.45 | A | C |
| ATOM | 321 | O | LYS | A | 595 | 1.042 | 20.388 | 3.438 | 1.00 | 34.47 | A | O |
| ATOM | 322 | N | VAL | A | 596 | 1.016 | 22.569 | 3.988 | 1.00 | 33.39 | A | N |
| ATOM | 323 | CA | VAL | A | 596 | 1.807 | 22.386 | 5.199 | 1.00 | 33.19 | A | C |
| ATOM | 324 | CB | VAL | A | 596 | 3.187 | 23.110 | 5.123 | 1.00 | 33.40 | A | C |
| ATOM | 325 | CG1 | VAL | A | 596 | 3.002 | 24.596 | 4.842 | 1.00 | 32.61 | A | C |
| ATOM | 326 | CG2 | VAL | A | 596 | 3.939 | 22.922 | 6.432 | 1.00 | 32.89 | A | C |
| ATOM | 327 | C | VAL | A | 596 | 1.015 | 22.938 | 6.372 | 1.00 | 34.53 | A | C |
| ATOM | 328 | O | VAL | A | 596 | 0.429 | 24.018 | 6.283 | 1.00 | 35.62 | A | O |
| ATOM | 329 | N | VAL | A | 597 | 0.986 | 22.189 | 7.469 | 1.00 | 35.15 | A | N |
| ATOM | 330 | CA | VAL | A | 597 | 0.249 | 22.611 | 8.650 | 1.00 | 36.74 | A | C |
| ATOM | 331 | CB | VAL | A | 597 | −0.964 | 21.687 | 8.925 | 1.00 | 36.15 | A | C |
| ATOM | 332 | CG1 | VAL | A | 597 | −1.814 | 21.543 | 7.674 | 1.00 | 35.98 | A | C |
| ATOM | 333 | CG2 | VAL | A | 597 | −0.481 | 20.328 | 9.408 | 1.00 | 38.07 | A | C |
| ATOM | 334 | C | VAL | A | 597 | 1.125 | 22.602 | 9.889 | 1.00 | 36.22 | A | C |
| ATOM | 335 | O | VAL | A | 597 | 2.092 | 21.847 | 9.979 | 1.00 | 37.18 | A | O |
| ATOM | 336 | N | GLU | A | 598 | 0.777 | 23.457 | 10.842 | 1.00 | 37.77 | A | N |
| ATOM | 337 | CA | GLU | A | 598 | 1.501 | 23.539 | 12.099 | 1.00 | 38.02 | A | C |
| ATOM | 338 | CB | GLU | A | 598 | 1.300 | 24.914 | 12.733 | 1.00 | 41.15 | A | C |
| ATOM | 339 | CG | GLU | A | 598 | 1.869 | 25.053 | 14.130 | 1.00 | 44.89 | A | C |
| ATOM | 340 | CD | GLU | A | 598 | 1.611 | 26.428 | 14.716 | 1.00 | 48.46 | A | C |
| ATOM | 341 | OE1 | GLU | A | 598 | 0.481 | 26.940 | 14.557 | 1.00 | 51.03 | A | O |
| ATOM | 342 | OE2 | GLU | A | 598 | 2.531 | 26.995 | 15.341 | 1.00 | 49.97 | A | O |
| ATOM | 343 | C | GLU | A | 598 | 0.915 | 22.455 | 12.991 | 1.00 | 39.95 | A | C |
| ATOM | 344 | O | GLU | A | 598 | −0.298 | 22.228 | 12.985 | 1.00 | 39.64 | A | O |
| ATOM | 345 | N | ALA | A | 599 | 1.771 | 21.781 | 13.749 | 1.00 | 40.04 | A | N |
| ATOM | 346 | CA | ALA | A | 599 | 1.308 | 20.717 | 14.622 | 1.00 | 42.50 | A | C |
| ATOM | 347 | CB | ALA | A | 599 | 1.184 | 19.415 | 13.832 | 1.00 | 40.30 | A | C |
| ATOM | 348 | C | ALA | A | 599 | 2.241 | 20.516 | 15.800 | 1.00 | 43.18 | A | C |
| ATOM | 349 | O | ALA | A | 599 | 3.380 | 20.983 | 15.792 | 1.00 | 43.96 | A | O |
| ATOM | 350 | N | THR | A | 600 | 1.741 | 19.823 | 16.818 | 1.00 | 44.62 | A | N |
| ATOM | 351 | CA | THR | A | 600 | 2.524 | 19.523 | 18.008 | 1.00 | 43.92 | A | C |
| ATOM | 352 | CB | THR | A | 600 | 1.733 | 19.824 | 19.306 | 1.00 | 45.49 | A | C |
| ATOM | 353 | OG1 | THR | A | 600 | 1.314 | 21.195 | 19.317 | 1.00 | 45.84 | A | O |
| ATOM | 354 | CG2 | THR | A | 600 | 2.598 | 19.554 | 20.525 | 1.00 | 43.01 | A | C |
| ATOM | 355 | C | THR | A | 600 | 2.848 | 18.033 | 17.978 | 1.00 | 44.17 | A | C |
| ATOM | 356 | O | THR | A | 600 | 1.956 | 17.208 | 17.803 | 1.00 | 44.96 | A | O |
| ATOM | 357 | N | ALA | A | 601 | 4.123 | 17.691 | 18.136 | 1.00 | 44.61 | A | N |
| ATOM | 358 | CA | ALA | A | 601 | 4.546 | 16.294 | 18.132 | 1.00 | 46.30 | A | C |
| ATOM | 359 | CB | ALA | A | 601 | 5.691 | 16.091 | 17.145 | 1.00 | 46.07 | A | C |
| ATOM | 360 | C | ALA | A | 601 | 4.990 | 15.905 | 19.535 | 1.00 | 47.25 | A | C |
| ATOM | 361 | O | ALA | A | 601 | 5.819 | 16.587 | 20.141 | 1.00 | 47.52 | A | O |
| ATOM | 362 | N | PHE | A | 602 | 4.444 | 14.808 | 20.050 | 1.00 | 47.57 | A | N |
| ATOM | 363 | CA | PHE | A | 602 | 4.788 | 14.355 | 21.393 | 1.00 | 47.95 | A | C |
| ATOM | 364 | CB | PHE | A | 602 | 3.523 | 13.938 | 22.153 | 1.00 | 46.66 | A | C |
| ATOM | 365 | CG | PHE | A | 602 | 2.417 | 14.954 | 22.094 | 1.00 | 45.97 | A | C |
| ATOM | 366 | CD1 | PHE | A | 602 | 1.493 | 14.935 | 21.055 | 1.00 | 45.11 | A | C |
| ATOM | 367 | CD2 | PHE | A | 602 | 2.306 | 15.940 | 23.071 | 1.00 | 45.23 | A | C |
| ATOM | 368 | CE1 | PHE | A | 602 | 0.472 | 15.882 | 20.989 | 1.00 | 45.31 | A | C |
| ATOM | 369 | CE2 | PHE | A | 602 | 1.289 | 16.892 | 23.014 | 1.00 | 45.31 | A | C |
| ATOM | 370 | CZ | PHE | A | 602 | 0.371 | 16.863 | 21.972 | 1.00 | 44.91 | A | C |
| ATOM | 371 | C | PHE | A | 602 | 5.781 | 13.199 | 21.374 | 1.00 | 47.96 | A | C |
| ATOM | 372 | O | PHE | A | 602 | 5.513 | 12.143 | 20.803 | 1.00 | 48.72 | A | O |
| ATOM | 373 | N | GLY | A | 603 | 6.928 | 13.407 | 22.012 | 1.00 | 48.76 | A | N |
| ATOM | 374 | CA | GLY | A | 603 | 7.944 | 12.374 | 22.055 | 1.00 | 49.43 | A | C |
| ATOM | 375 | C | GLY | A | 603 | 8.622 | 12.206 | 20.709 | 1.00 | 50.41 | A | C |
| ATOM | 376 | O | GLY | A | 603 | 8.557 | 11.135 | 20.104 | 1.00 | 50.27 | A | O |
| ATOM | 377 | N | LEU | A | 604 | 9.273 | 13.267 | 20.241 | 1.00 | 50.77 | A | N |
| ATOM | 378 | CA | LEU | A | 604 | 9.961 | 13.236 | 18.954 | 1.00 | 52.26 | A | C |
| ATOM | 379 | CB | LEU | A | 604 | 9.449 | 14.372 | 18.057 | 1.00 | 52.68 | A | C |
| ATOM | 380 | CG | LEU | A | 604 | 9.776 | 14.349 | 16.557 | 1.00 | 53.44 | A | C |
| ATOM | 381 | CD1 | LEU | A | 604 | 9.064 | 15.512 | 15.874 | 1.00 | 53.80 | A | C |
| ATOM | 382 | CD2 | LEU | A | 604 | 11.272 | 14.441 | 16.333 | 1.00 | 53.47 | A | C |
| ATOM | 383 | C | LEU | A | 604 | 11.463 | 13.375 | 19.156 | 1.00 | 52.36 | A | C |
| ATOM | 384 | O | LEU | A | 604 | 11.922 | 14.269 | 19.868 | 1.00 | 52.30 | A | O |
| ATOM | 385 | N | GLY | A | 605 | 12.224 | 12.487 | 18.524 | 1.00 | 53.62 | A | N |
| ATOM | 386 | CA | GLY | A | 605 | 13.669 | 12.535 | 18.643 | 1.00 | 55.92 | A | C |
| ATOM | 387 | C | GLY | A | 605 | 14.183 | 11.789 | 19.858 | 1.00 | 57.76 | A | C |
| ATOM | 388 | O | GLY | A | 605 | 13.411 | 11.167 | 20.588 | 1.00 | 57.37 | A | O |
| ATOM | 389 | N | LYS | A | 606 | 15.491 | 11.856 | 20.079 | 1.00 | 60.19 | A | N |
| ATOM | 390 | CA | LYS | A | 606 | 16.112 | 11.178 | 21.209 | 1.00 | 62.68 | A | C |
| ATOM | 391 | CB | LYS | A | 606 | 17.618 | 11.037 | 20.969 | 1.00 | 63.52 | A | C |
| ATOM | 392 | CG | LYS | A | 606 | 18.189 | 9.666 | 21.331 | 1.00 | 65.35 | A | C |
| ATOM | 393 | CD | LYS | A | 606 | 18.010 | 9.330 | 22.807 | 1.00 | 66.31 | A | C |
| ATOM | 394 | CE | LYS | A | 606 | 18.804 | 10.275 | 23.703 | 1.00 | 67.06 | A | C |
| ATOM | 395 | NZ | LYS | A | 606 | 18.564 | 10.002 | 25.152 | 1.00 | 65.76 | A | N |
| ATOM | 396 | C | LYS | A | 606 | 15.861 | 11.946 | 22.506 | 1.00 | 63.77 | A | C |
| ATOM | 397 | O | LYS | A | 606 | 16.037 | 11.409 | 23.599 | 1.00 | 64.44 | A | O |
| ATOM | 398 | N | GLU | A | 607 | 15.444 | 13.202 | 22.377 | 1.00 | 64.49 | A | N |
| ATOM | 399 | CA | GLU | A | 607 | 15.168 | 14.042 | 23.538 | 1.00 | 65.19 | A | C |
| ATOM | 400 | CB | GLU | A | 607 | 15.553 | 15.494 | 23.238 | 1.00 | 66.00 | A | C |
| ATOM | 401 | CG | GLU | A | 607 | 17.046 | 15.705 | 23.041 | 1.00 | 67.13 | A | C |
| ATOM | 402 | CD | GLU | A | 607 | 17.852 | 15.285 | 24.257 | 1.00 | 68.09 | A | C |
| ATOM | 403 | OE1 | GLU | A | 607 | 17.654 | 15.883 | 25.336 | 1.00 | 68.20 | A | O |
| ATOM | 404 | OE2 | GLU | A | 607 | 18.681 | 14.356 | 24.136 | 1.00 | 68.54 | A | O |
| ATOM | 405 | C | GLU | A | 607 | 13.703 | 13.973 | 23.965 | 1.00 | 65.19 | A | C |
| ATOM | 406 | O | GLU | A | 607 | 13.310 | 14.582 | 24.961 | 1.00 | 65.12 | A | O |
| ATOM | 407 | N | ASP | A | 608 | 12.903 | 13.226 | 23.208 | 1.00 | 64.95 | A | N |
| ATOM | 408 | CA | ASP | A | 608 | 11.482 | 13.063 | 23.501 | 1.00 | 64.78 | A | C |
| ATOM | 409 | CB | ASP | A | 608 | 11.302 | 12.179 | 24.740 | 1.00 | 66.39 | A | C |
| ATOM | 410 | CG | ASP | A | 608 | 11.868 | 10.785 | 24.548 | 1.00 | 67.46 | A | C |
| ATOM | 411 | OD1 | ASP | A | 608 | 11.756 | 9.960 | 25.479 | 1.00 | 68.46 | A | O |
| ATOM | 412 | OD2 | ASP | A | 608 | 12.426 | 10.511 | 23.465 | 1.00 | 69.08 | A | O |
| ATOM | 413 | C | ASP | A | 608 | 10.786 | 14.403 | 23.722 | 1.00 | 64.02 | A | C |
| ATOM | 414 | O | ASP | A | 608 | 9.998 | 14.559 | 24.655 | 1.00 | 63.43 | A | O |
| ATOM | 415 | N | ALA | A | 609 | 11.074 | 15.366 | 22.855 | 1.00 | 62.73 | A | N |
| ATOM | 416 | CA | ALA | A | 609 | 10.481 | 16.691 | 22.967 | 1.00 | 61.09 | A | C |
| ATOM | 417 | CB | ALA | A | 609 | 11.277 | 17.688 | 22.132 | 1.00 | 61.62 | A | C |
| ATOM | 418 | C | ALA | A | 609 | 9.020 | 16.697 | 22.533 | 1.00 | 60.38 | A | C |
| ATOM | 419 | O | ALA | A | 609 | 8.545 | 15.765 | 21.883 | 1.00 | 60.12 | A | O |
| ATOM | 420 | N | VAL | A | 610 | 8.314 | 17.760 | 22.905 | 1.00 | 58.44 | A | N |
| ATOM | 421 | CA | VAL | A | 610 | 6.909 | 17.927 | 22.559 | 1.00 | 57.44 | A | C |
| ATOM | 422 | CB | VAL | A | 610 | 6.066 | 18.195 | 23.827 | 1.00 | 57.02 | A | C |
| ATOM | 423 | CG1 | VAL | A | 610 | 4.599 | 18.328 | 23.470 | 1.00 | 56.89 | A | C |
| ATOM | 424 | CG2 | VAL | A | 610 | 6.269 | 17.060 | 24.821 | 1.00 | 56.92 | A | C |
| ATOM | 425 | C | VAL | A | 610 | 6.808 | 19.115 | 21.601 | 1.00 | 56.74 | A | C |
| ATOM | 426 | O | VAL | A | 610 | 5.720 | 19.555 | 21.227 | 1.00 | 57.00 | A | O |
| ATOM | 427 | N | LEU | A | 611 | 7.975 | 19.610 | 21.203 | 1.00 | 55.57 | A | N |
| ATOM | 428 | CA | LEU | A | 611 | 8.107 | 20.744 | 20.297 | 1.00 | 52.67 | A | C |
| ATOM | 429 | CB | LEU | A | 611 | 9.480 | 20.700 | 19.619 | 1.00 | 55.04 | A | C |
| ATOM | 430 | CG | LEU | A | 611 | 9.975 | 19.331 | 19.134 | 1.00 | 55.70 | A | C |
| ATOM | 431 | CD1 | LEU | A | 611 | 8.909 | 18.641 | 18.301 | 1.00 | 55.90 | A | C |
| ATOM | 432 | CD2 | LEU | A | 611 | 11.251 | 19.517 | 18.328 | 1.00 | 56.99 | A | C |
| ATOM | 433 | C | LEU | A | 611 | 7.038 | 20.900 | 19.225 | 1.00 | 50.88 | A | C |
| ATOM | 434 | O | LEU | A | 611 | 6.365 | 19.944 | 18.834 | 1.00 | 49.85 | A | O |
| ATOM | 435 | N | LYS | A | 612 | 6.897 | 22.134 | 18.755 | 1.00 | 47.99 | A | N |
| ATOM | 436 | CA | LYS | A | 612 | 5.949 | 22.461 | 17.704 | 1.00 | 45.40 | A | C |
| ATOM | 437 | CB | LYS | A | 612 | 5.578 | 23.942 | 17.782 | 1.00 | 47.84 | A | C |
| ATOM | 438 | CG | LYS | A | 612 | 4.305 | 24.304 | 17.053 | 1.00 | 50.82 | A | C |
| ATOM | 439 | CD | LYS | A | 612 | 3.826 | 25.682 | 17.481 | 1.00 | 53.52 | A | C |
| ATOM | 440 | CE | LYS | A | 612 | 2.335 | 25.669 | 17.788 | 1.00 | 56.32 | A | C |
| ATOM | 441 | NZ | LYS | A | 612 | 1.835 | 27.026 | 18.143 | 1.00 | 57.98 | A | N |
| ATOM | 442 | C | LYS | A | 612 | 6.704 | 22.164 | 16.412 | 1.00 | 41.45 | A | C |
| ATOM | 443 | O | LYS | A | 612 | 7.908 | 22.393 | 16.335 | 1.00 | 39.69 | A | O |
| ATOM | 444 | N | VAL | A | 613 | 6.008 | 21.647 | 15.408 | 1.00 | 39.42 | A | N |
| ATOM | 445 | CA | VAL | A | 613 | 6.651 | 21.304 | 14.148 | 1.00 | 35.63 | A | C |
| ATOM | 446 | CB | VAL | A | 613 | 7.005 | 19.794 | 14.091 | 1.00 | 34.92 | A | C |
| ATOM | 447 | CG1 | VAL | A | 613 | 8.071 | 19.464 | 15.115 | 1.00 | 34.86 | A | C |
| ATOM | 448 | CG2 | VAL | A | 613 | 5.757 | 18.960 | 14.335 | 1.00 | 35.32 | A | C |
| ATOM | 449 | C | VAL | A | 613 | 5.779 | 21.621 | 12.950 | 1.00 | 34.73 | A | C |
| ATOM | 450 | O | VAL | A | 613 | 4.626 | 22.026 | 13.092 | 1.00 | 33.73 | A | O |
| ATOM | 451 | N | ALA | A | 614 | 6.340 | 21.431 | 11.761 | 1.00 | 32.99 | A | N |
| ATOM | 452 | CA | ALA | A | 614 | 5.597 | 21.671 | 10.533 | 1.00 | 32.60 | A | C |
| ATOM | 453 | CB | ALA | A | 614 | 6.383 | 22.584 | 9.614 | 1.00 | 30.37 | A | C |
| ATOM | 454 | C | ALA | A | 614 | 5.367 | 20.317 | 9.873 | 1.00 | 32.30 | A | C |
| ATOM | 455 | O | ALA | A | 614 | 6.269 | 19.479 | 9.832 | 1.00 | 32.96 | A | O |
| ATOM | 456 | N | VAL | A | 615 | 4.156 | 20.101 | 9.373 | 1.00 | 31.44 | A | N |
| ATOM | 457 | CA | VAL | A | 615 | 3.822 | 18.837 | 8.730 | 1.00 | 30.85 | A | C |
| ATOM | 458 | CB | VAL | A | 615 | 2.754 | 18.062 | 9.553 | 1.00 | 30.95 | A | C |
| ATOM | 459 | CG1 | VAL | A | 615 | 2.453 | 16.715 | 8.901 | 1.00 | 30.88 | A | C |
| ATOM | 460 | CG2 | VAL | A | 615 | 3.250 | 17.854 | 10.969 | 1.00 | 29.81 | A | C |
| ATOM | 461 | C | VAL | A | 615 | 3.321 | 19.063 | 7.311 | 1.00 | 30.31 | A | C |
| ATOM | 462 | O | VAL | A | 615 | 2.290 | 19.705 | 7.099 | 1.00 | 30.36 | A | O |
| ATOM | 463 | N | LYS | A | 616 | 4.078 | 18.550 | 6.342 | 1.00 | 29.30 | A | N |
| ATOM | 464 | CA | LYS | A | 616 | 3.733 | 18.672 | 4.930 | 1.00 | 32.42 | A | C |
| ATOM | 465 | CB | LYS | A | 616 | 5.003 | 18.775 | 4.073 | 1.00 | 33.22 | A | C |
| ATOM | 466 | CG | LYS | A | 616 | 6.037 | 19.740 | 4.620 | 1.00 | 37.03 | A | C |
| ATOM | 467 | CD | LYS | A | 616 | 7.297 | 19.792 | 3.769 | 1.00 | 39.55 | A | C |
| ATOM | 468 | CE | LYS | A | 616 | 8.372 | 20.599 | 4.478 | 1.00 | 39.35 | A | C |
| ATOM | 469 | NZ | LYS | A | 616 | 9.538 | 20.889 | 3.611 | 1.00 | 46.23 | A | N |
| ATOM | 470 | C | LYS | A | 616 | 2.957 | 17.425 | 4.522 | 1.00 | 33.00 | A | C |
| ATOM | 471 | O | LYS | A | 616 | 3.294 | 16.318 | 4.938 | 1.00 | 32.17 | A | O |
| ATOM | 472 | N | MET | A | 617 | 1.930 | 17.613 | 3.700 | 1.00 | 33.20 | A | N |
| ATOM | 473 | CA | MET | A | 617 | 1.098 | 16.509 | 3.235 | 1.00 | 35.19 | A | C |
| ATOM | 474 | CB | MET | A | 617 | 0.010 | 16.215 | 4.269 | 1.00 | 36.09 | A | C |
| ATOM | 475 | CG | MET | A | 617 | −0.920 | 17.396 | 4.512 | 1.00 | 38.89 | A | C |
| ATOM | 476 | SD | MET | A | 617 | −1.951 | 17.179 | 5.964 | 1.00 | 41.81 | A | S |
| ATOM | 477 | CE | MET | A | 617 | −0.859 | 17.755 | 7.191 | 1.00 | 40.42 | A | C |
| ATOM | 478 | C | MET | A | 617 | 0.452 | 16.868 | 1.902 | 1.00 | 35.81 | A | C |
| ATOM | 479 | O | MET | A | 617 | 0.390 | 18.035 | 1.529 | 1.00 | 34.69 | A | O |
| ATOM | 480 | N | LEU | A | 618 | −0.026 | 15.858 | 1.186 | 1.00 | 38.40 | A | N |
| ATOM | 481 | CA | LEU | A | 618 | −0.669 | 16.077 | −0.102 | 1.00 | 42.31 | A | C |
| ATOM | 482 | CB | LEU | A | 618 | −0.519 | 14.833 | −0.975 | 1.00 | 42.77 | A | C |
| ATOM | 483 | CG | LEU | A | 618 | 0.928 | 14.407 | −1.242 | 1.00 | 44.34 | A | C |
| ATOM | 484 | CD1 | LEU | A | 618 | 0.949 | 13.070 | −1.962 | 1.00 | 44.42 | A | C |
| ATOM | 485 | CD2 | LEU | A | 618 | 1.628 | 15.476 | −2.060 | 1.00 | 44.18 | A | C |
| ATOM | 486 | C | LEU | A | 618 | −2.150 | 16.404 | 0.073 | 1.00 | 44.15 | A | C |
| ATOM | 487 | O | LEU | A | 618 | −2.719 | 16.203 | 1.150 | 1.00 | 44.39 | A | O |
| ATOM | 488 | N | LYS | A | 619 | −2.760 | 16.930 | −0.986 | 1.00 | 46.55 | A | N |
| ATOM | 489 | CA | LYS | A | 619 | −4.177 | 17.266 | −0.973 | 1.00 | 49.19 | A | C |
| ATOM | 490 | CB | LYS | A | 619 | −4.423 | 18.646 | −1.596 | 1.00 | 50.25 | A | C |
| ATOM | 491 | CG | LYS | A | 619 | −4.101 | 19.820 | −0.678 | 1.00 | 51.84 | A | C |
| ATOM | 492 | CD | LYS | A | 619 | −4.637 | 21.133 | −1.240 | 1.00 | 53.72 | A | C |
| ATOM | 493 | CE | LYS | A | 619 | −4.481 | 22.272 | −0.239 | 1.00 | 53.16 | A | C |
| ATOM | 494 | NZ | LYS | A | 619 | −5.126 | 23.532 | −0.716 | 1.00 | 54.94 | A | N |
| ATOM | 495 | C | LYS | A | 619 | −4.945 | 16.207 | −1.755 | 1.00 | 51.05 | A | C |
| ATOM | 496 | O | LYS | A | 619 | −4.348 | 15.335 | −2.388 | 1.00 | 50.59 | A | O |
| ATOM | 497 | N | SER | A | 620 | −6.271 | 16.294 | −1.713 | 1.00 | 53.46 | A | N |
| ATOM | 498 | CA | SER | A | 620 | −7.137 | 15.345 | −2.408 | 1.00 | 55.58 | A | C |
| ATOM | 499 | CB | SER | A | 620 | −8.604 | 15.677 | −2.117 | 1.00 | 55.37 | A | C |
| ATOM | 500 | OG | SER | A | 620 | 8.848 | 15.726 | −0.722 | 1.00 | 56.91 | A | O |
| ATOM | 501 | C | SER | A | 620 | 6.914 | 15.343 | −3.917 | 1.00 | 56.82 | A | C |
| ATOM | 502 | O | SER | A | 620 | −7.111 | 14.326 | −4.583 | 1.00 | 56.85 | A | O |
| ATOM | 503 | N | THR | A | 621 | −6.498 | 16.486 | −4.449 | 1.00 | 58.76 | A | N |
| ATOM | 504 | CA | THR | A | 621 | −6.271 | 16.635 | −5.883 | 1.00 | 61.23 | A | C |
| ATOM | 505 | CB | THR | A | 621 | −6.330 | 18.126 | −6.289 | 1.00 | 61.84 | A | C |
| ATOM | 506 | OG1 | THR | A | 621 | −7.430 | 18.760 | −5.625 | 1.00 | 62.57 | A | O |
| ATOM | 507 | CG2 | THR | A | 621 | −6.525 | 18.266 | −7.794 | 1.00 | 62.34 | A | C |
| ATOM | 508 | C | THR | A | 621 | −4.932 | 16.059 | −6.342 | 1.00 | 62.19 | A | C |
| ATOM | 509 | O | THR | A | 621 | −4.548 | 16.223 | −7.498 | 1.00 | 62.92 | A | O |
| ATOM | 510 | N | ALA | A | 622 | −4.228 | 15.375 | −5.445 | 1.00 | 63.47 | A | N |
| ATOM | 511 | CA | ALA | A | 622 | −2.927 | 14.800 | −5.782 | 1.00 | 64.25 | A | C |
| ATOM | 512 | CB | ALA | A | 622 | −1.956 | 15.010 | −4.624 | 1.00 | 64.52 | A | C |
| ATOM | 513 | C | ALA | A | 622 | −2.992 | 13.319 | −6.140 | 1.00 | 65.13 | A | C |
| ATOM | 514 | O | ALA | A | 622 | −3.807 | 12.572 | −5.599 | 1.00 | 65.10 | A | O |
| ATOM | 515 | N | HIS | A | 623 | −2.119 | 12.902 | −7.054 | 1.00 | 66.01 | A | N |
| ATOM | 516 | CA | HIS | A | 623 | −2.055 | 11.508 | −7.486 | 1.00 | 67.66 | A | C |
| ATOM | 517 | CB | HIS | A | 623 | −2.491 | 11.378 | −8.949 | 1.00 | 69.58 | A | C |
| ATOM | 518 | CG | HIS | A | 623 | −3.953 | 11.115 | −9.121 | 1.00 | 71.78 | A | C |
| ATOM | 519 | CD2 | HIS | A | 623 | −4.915 | 11.809 | −9.776 | 1.00 | 72.43 | A | C |
| ATOM | 520 | ND1 | HIS | A | 623 | −4.577 | 10.010 | −8.583 | 1.00 | 72.69 | A | N |
| ATOM | 521 | CE1 | HIS | A | 623 | −5.860 | 10.033 | −8.898 | 1.00 | 73.42 | A | C |
| ATOM | 522 | NE2 | HIS | A | 623 | −6.091 | 11.115 | −9.622 | 1.00 | 73.70 | A | N |
| ATOM | 523 | C | HIS | A | 623 | −0.658 | 10.921 | −7.319 | 1.00 | 67.21 | A | C |
| ATOM | 524 | O | HIS | A | 623 | 0.169 | 11.460 | −6.585 | 1.00 | 66.93 | A | O |
| ATOM | 525 | N | ALA | A | 624 | −0.407 | 9.811 | −8.007 | 1.00 | 66.82 | A | N |
| ATOM | 526 | CA | ALA | A | 624 | 0.881 | 9.129 | −7.946 | 1.00 | 66.51 | A | C |
| ATOM | 527 | CB | ALA | A | 624 | 0.964 | 8.082 | −9.052 | 1.00 | 66.37 | A | C |
| ATOM | 528 | C | ALA | A | 624 | 2.055 | 10.097 | −8.058 | 1.00 | 66.01 | A | C |
| ATOM | 529 | O | ALA | A | 624 | 2.837 | 10.244 | −7.122 | 1.00 | 66.23 | A | O |
| ATOM | 530 | N | ASP | A | 625 | 2.168 | 10.751 | −9.211 | 1.00 | 65.20 | A | N |
| ATOM | 531 | CA | ASP | A | 625 | 3.245 | 11.704 | −9.470 | 1.00 | 64.04 | A | C |
| ATOM | 532 | CB | ASP | A | 625 | 2.921 | 12.522 | −10.721 | 1.00 | 65.31 | A | C |
| ATOM | 533 | CG | ASP | A | 625 | 2.650 | 11.651 | −11.931 | 1.00 | 66.37 | A | C |
| ATOM | 534 | OD1 | ASP | A | 625 | 2.287 | 12.202 | −12.991 | 1.00 | 67.06 | A | O |
| ATOM | 535 | OD2 | ASP | A | 625 | 2.802 | 10.416 | −11.822 | 1.00 | 67.63 | A | O |
| ATOM | 536 | C | ASP | A | 625 | 3.478 | 12.648 | −8.294 | 1.00 | 62.48 | A | C |
| ATOM | 537 | O | ASP | A | 625 | 4.620 | 12.943 | −7.937 | 1.00 | 61.99 | A | O |
| ATOM | 538 | N | GLU | A | 626 | 2.387 | 13.122 | −7.701 | 1.00 | 60.47 | A | N |
| ATOM | 539 | CA | GLU | A | 626 | 2.458 | 14.034 | −6.565 | 1.00 | 58.18 | A | C |
| ATOM | 540 | CB | GLU | A | 626 | 1.056 | 14.522 | −6.192 | 1.00 | 58.94 | A | C |
| ATOM | 541 | CG | GLU | A | 626 | 0.183 | 14.882 | −7.378 | 1.00 | 60.04 | A | C |
| ATOM | 542 | CD | GLU | A | 626 | 0.852 | 15.858 | −8.312 | 1.00 | 60.48 | A | C |
| ATOM | 543 | OE1 | GLU | A | 626 | 1.241 | 16.948 | −7.848 | 1.00 | 61.70 | A | O |
| ATOM | 544 | OE2 | GLU | A | 626 | 0.991 | 15.536 | −9.510 | 1.00 | 61.36 | A | O |
| ATOM | 545 | C | GLU | A | 626 | 3.074 | 13.323 | −5.364 | 1.00 | 55.66 | A | C |
| ATOM | 546 | O | GLU | A | 626 | 3.983 | 13.844 | −4.719 | 1.00 | 54.16 | A | O |
| ATOM | 547 | N | LYS | A | 627 | 2.562 | 12.131 | −5.077 | 1.00 | 52.99 | A | N |
| ATOM | 548 | CA | LYS | A | 627 | 3.034 | 11.324 | −3.959 | 1.00 | 51.38 | A | C |
| ATOM | 549 | CB | LYS | A | 627 | 2.236 | 10.020 | −3.877 | 1.00 | 51.94 | A | C |
| ATOM | 550 | CG | LYS | A | 627 | 0.754 | 10.213 | −3.601 | 1.00 | 54.04 | A | C |
| ATOM | 551 | CD | LYS | A | 627 | 0.035 | 8.879 | −3.474 | 1.00 | 55.76 | A | C |
| ATOM | 552 | CE | LYS | A | 627 | −1.425 | 9.068 | −3.094 | 1.00 | 56.04 | A | C |
| ATOM | 553 | NZ | LYS | A | 627 | −2.164 | 9.867 | −4.106 | 1.00 | 56.84 | A | N |
| ATOM | 554 | C | LYS | A | 627 | 4.518 | 11.007 | −4.081 | 1.00 | 49.13 | A | C |
| ATOM | 555 | O | LYS | A | 627 | 5.231 | 10.956 | −3.081 | 1.00 | 46.60 | A | O |
| ATOM | 556 | N | GLU | A | 628 | 4.978 | 10.789 | −5.309 | 1.00 | 48.11 | A | N |
| ATOM | 557 | CA | GLU | A | 628 | 6.383 | 10.484 | −5.544 | 1.00 | 47.77 | A | C |
| ATOM | 558 | CB | GLU | A | 628 | 6.598 | 10.040 | −6.997 | 1.00 | 50.42 | A | C |
| ATOM | 559 | CG | GLU | A | 628 | 6.044 | 8.654 | −7.300 | 1.00 | 54.62 | A | C |
| ATOM | 560 | CD | GLU | A | 628 | 6.242 | 8.237 | −8.748 | 1.00 | 58.25 | A | C |
| ATOM | 561 | OE1 | GLU | A | 628 | 7.386 | 8.331 | −9.246 | 1.00 | 60.00 | A | O |
| ATOM | 562 | OE2 | GLU | A | 628 | 5.255 | 7.806 | −9.386 | 1.00 | 59.90 | A | O |
| ATOM | 563 | C | GLU | A | 628 | 7.260 | 11.691 | −5.227 | 1.00 | 45.63 | A | C |
| ATOM | 564 | O | GLU | A | 628 | 8.309 | 11.554 | −4.597 | 1.00 | 45.97 | A | O |
| ATOM | 565 | N | ALA | A | 629 | 6.823 | 12.872 | −5.657 | 1.00 | 42.99 | A | N |
| ATOM | 566 | CA | ALA | A | 629 | 7.574 | 14.099 | −5.409 | 1.00 | 40.68 | A | C |
| ATOM | 567 | CB | ALA | A | 629 | 6.858 | 15.294 | −6.042 | 1.00 | 42.09 | A | C |
| ATOM | 568 | C | ALA | A | 629 | 7.759 | 14.332 | −3.911 | 1.00 | 38.22 | A | C |
| ATOM | 569 | O | ALA | A | 629 | 8.815 | 14.798 | −3.474 | 1.00 | 37.70 | A | O |
| ATOM | 570 | N | LEU | A | 630 | 6.734 | 14.016 | −3.126 | 1.00 | 34.33 | A | N |
| ATOM | 571 | CA | LEU | A | 630 | 6.815 | 14.197 | −1.679 | 1.00 | 33.68 | A | C |
| ATOM | 572 | CB | LEU | A | 630 | 5.442 | 13.998 | −1.033 | 1.00 | 33.94 | A | C |
| ATOM | 573 | CG | LEU | A | 630 | 5.355 | 14.290 | 0.467 | 1.00 | 33.78 | A | C |
| ATOM | 574 | OD1 | LEU | A | 630 | 5.812 | 15.716 | 0.749 | 1.00 | 33.38 | A | C |
| ATOM | 575 | CD2 | LEU | A | 630 | 3.925 | 14.078 | 0.938 | 1.00 | 36.00 | A | C |
| ATOM | 576 | C | LEU | A | 630 | 7.817 | 13.206 | −1.083 | 1.00 | 32.83 | A | C |
| ATOM | 577 | O | LEU | A | 630 | 8.607 | 13.556 | −0.207 | 1.00 | 31.87 | A | O |
| ATOM | 578 | N | MET | A | 631 | 7.781 | 11.970 | −1.569 | 1.00 | 32.86 | A | N |
| ATOM | 579 | CA | MET | A | 631 | 8.699 | 10.941 | −1.097 | 1.00 | 32.19 | A | C |
| ATOM | 580 | CB | MET | A | 631 | 8.306 | 9.570 | −1.659 | 1.00 | 33.41 | A | C |
| ATOM | 581 | CG | MET | A | 631 | 7.084 | 8.940 | −1.003 | 1.00 | 35.18 | A | C |
| ATOM | 582 | SD | MET | A | 631 | 7.240 | 8.744 | 0.797 | 1.00 | 38.35 | A | S |
| ATOM | 583 | CE | MET | A | 631 | 8.831 | 7.916 | 0.934 | 1.00 | 35.95 | A | C |
| ATOM | 584 | C | MET | A | 631 | 10.123 | 11.290 | −1.526 | 1.00 | 32.73 | A | C |
| ATOM | 585 | O | MET | A | 631 | 11.072 | 11.044 | −0.790 | 1.00 | 33.91 | A | O |
| ATOM | 586 | N | SER | A | 632 | 10.264 | 11.870 | −2.716 | 1.00 | 32.45 | A | N |
| ATOM | 587 | CA | SER | A | 632 | 11.578 | 12.264 | −3.215 | 1.00 | 33.43 | A | C |
| ATOM | 588 | CB | SER | A | 632 | 11.489 | 12.734 | −4.672 | 1.00 | 33.95 | A | C |
| ATOM | 589 | OG | SER | A | 632 | 11.235 | 11.645 | −5.541 | 1.00 | 39.34 | A | O |
| ATOM | 590 | C | SER | A | 632 | 12.178 | 13.373 | −2.356 | 1.00 | 32.55 | A | C |
| ATOM | 591 | O | SER | A | 632 | 13.381 | 13.381 | −2.092 | 1.00 | 32.51 | A | O |
| ATOM | 592 | N | GLU | A | 633 | 11.345 | 14.313 | −1.923 | 1.00 | 33.05 | A | N |
| ATOM | 593 | CA | GLU | A | 633 | 11.838 | 15.396 | −1.082 | 1.00 | 32.69 | A | C |
| ATOM | 594 | CB | GLU | A | 633 | 10.739 | 16.433 | −0.832 | 1.00 | 35.18 | A | C |
| ATOM | 595 | CG | GLU | A | 633 | 11.219 | 17.650 | −0.046 | 1.00 | 38.91 | A | C |
| ATOM | 596 | CD | GLU | A | 633 | 10.087 | 18.571 | 0.358 | 1.00 | 41.03 | A | C |
| ATOM | 597 | OE1 | GLU | A | 633 | 9.216 | 18.842 | −0.492 | 1.00 | 41.88 | A | O |
| ATOM | 598 | OE2 | GLU | A | 633 | 10.074 | 19.030 | 1.522 | 1.00 | 43.76 | A | O |
| ATOM | 599 | C | GLU | A | 633 | 12.270 | 14.784 | 0.245 | 1.00 | 31.23 | A | C |
| ATOM | 600 | O | GLU | A | 633 | 13.307 | 15.145 | 0.811 | 1.00 | 29.89 | A | O |
| ATOM | 601 | N | LEU | A | 634 | 11.460 | 13.849 | 0.730 | 1.00 | 29.69 | A | N |
| ATOM | 602 | CA | LEU | A | 634 | 11.730 | 13.166 | 1.989 | 1.00 | 31.05 | A | C |
| ATOM | 603 | CB | LEU | A | 634 | 10.661 | 12.103 | 2.247 | 1.00 | 32.88 | A | C |
| ATOM | 604 | CG | LEU | A | 634 | 10.248 | 11.777 | 3.685 | 1.00 | 34.71 | A | C |
| ATOM | 605 | CD1 | LEU | A | 634 | 9.460 | 10.467 | 3.674 | 1.00 | 31.29 | A | C |
| ATOM | 606 | CD2 | LEU | A | 634 | 11.454 | 11.667 | 4.591 | 1.00 | 32.79 | A | C |
| ATOM | 607 | C | LEU | A | 634 | 13.098 | 12.495 | 1.894 | 1.00 | 30.54 | A | C |
| ATOM | 608 | O | LEU | A | 634 | 13.926 | 12.606 | 2.798 | 1.00 | 30.33 | A | O |
| ATOM | 609 | N | LYS | A | 635 | 13.326 | 11.810 | 0.778 | 1.00 | 31.01 | A | N |
| ATOM | 610 | CA | LYS | A | 635 | 14.583 | 11.110 | 0.543 | 1.00 | 31.88 | A | C |
| ATOM | 611 | CB | LYS | A | 635 | 14.476 | 10.270 | −0.731 | 1.00 | 32.92 | A | C |
| ATOM | 612 | CG | LYS | A | 635 | 13.453 | 9.143 | −0.638 | 1.00 | 34.16 | A | C |
| ATOM | 613 | CD | LYS | A | 635 | 13.221 | 8.488 | −1.988 | 1.00 | 36.67 | A | C |
| ATOM | 614 | CE | LYS | A | 635 | 12.230 | 7.332 | −1.889 | 1.00 | 38.08 | A | C |
| ATOM | 615 | NZ | LYS | A | 635 | 11.850 | 6.801 | −3.234 | 1.00 | 37.93 | A | N |
| ATOM | 616 | C | LYS | A | 635 | 15.776 | 12.061 | 0.453 | 1.00 | 30.50 | A | C |
| ATOM | 617 | O | LYS | A | 635 | 16.845 | 11.761 | 0.970 | 1.00 | 29.89 | A | O |
| ATOM | 618 | N | ILE | A | 636 | 15.596 | 13.204 | −0.200 | 1.00 | 30.73 | A | N |
| ATOM | 619 | CA | ILE | A | 636 | 16.681 | 14.179 | −0.321 | 1.00 | 31.50 | A | C |
| ATOM | 620 | CB | ILE | A | 636 | 16.277 | 15.369 | −1.230 | 1.00 | 31.60 | A | C |
| ATOM | 621 | CG2 | ILE | A | 636 | 17.237 | 16.543 | −1.030 | 1.00 | 32.33 | A | C |
| ATOM | 622 | CG1 | ILE | A | 636 | 16.254 | 14.915 | −2.690 | 1.00 | 32.86 | A | C |
| ATOM | 623 | CD1 | ILE | A | 636 | 15.765 | 15.968 | −3.665 | 1.00 | 33.04 | A | C |
| ATOM | 624 | C | ILE | A | 636 | 17.068 | 14.721 | 1.047 | 1.00 | 30.67 | A | C |
| ATOM | 625 | O | ILE | A | 636 | 18.249 | 14.766 | 1.402 | 1.00 | 30.22 | A | O |
| ATOM | 626 | N | MET | A | 637 | 16.062 | 15.126 | 1.815 | 1.00 | 30.60 | A | N |
| ATOM | 627 | CA | MET | A | 637 | 16.293 | 15.677 | 3.139 | 1.00 | 31.79 | A | C |
| ATOM | 628 | CB | MET | A | 637 | 14.973 | 16.165 | 3.745 | 1.00 | 30.60 | A | C |
| ATOM | 629 | CG | MET | A | 637 | 14.345 | 17.341 | 2.986 | 1.00 | 31.95 | A | C |
| ATOM | 630 | SD | MET | A | 637 | 15.555 | 18.656 | 2.640 | 1.00 | 33.78 | A | S |
| ATOM | 631 | CE | MET | A | 637 | 15.838 | 19.299 | 4.301 | 1.00 | 30.32 | A | C |
| ATOM | 632 | C | MET | A | 637 | 16.959 | 14.663 | 4.065 | 1.00 | 31.88 | A | C |
| ATOM | 633 | O | MET | A | 637 | 17.810 | 15.030 | 4.873 | 1.00 | 32.59 | A | O |
| ATOM | 634 | N | SER | A | 638 | 16.584 | 13.392 | 3.943 | 1.00 | 32.67 | A | N |
| ATOM | 635 | CA | SER | A | 638 | 17.171 | 12.356 | 4.789 | 1.00 | 36.16 | A | C |
| ATOM | 636 | CB | SER | A | 638 | 16.370 | 11.049 | 4.680 | 1.00 | 36.46 | A | C |
| ATOM | 637 | OG | SER | A | 638 | 16.485 | 10.468 | 3.390 | 1.00 | 37.37 | A | O |
| ATOM | 638 | C | SER | A | 638 | 18.633 | 12.098 | 4.414 | 1.00 | 38.27 | A | C |
| ATOM | 639 | O | SER | A | 638 | 19.405 | 11.585 | 5.222 | 1.00 | 39.47 | A | O |
| ATOM | 640 | N | HIS | A | 639 | 19.003 | 12.470 | 3.192 | 1.00 | 39.05 | A | N |
| ATOM | 641 | CA | HIS | A | 639 | 20.361 | 12.275 | 2.687 | 1.00 | 40.58 | A | C |
| ATOM | 642 | CB | HIS | A | 639 | 20.316 | 11.982 | 1.182 | 1.00 | 43.96 | A | C |
| ATOM | 643 | CG | HIS | A | 639 | 21.631 | 12.163 | 0.487 | 1.00 | 48.30 | A | C |
| ATOM | 644 | CD2 | HIS | A | 639 | 21.997 | 12.983 | −0.528 | 1.00 | 50.50 | A | C |
| ATOM | 645 | ND1 | HIS | A | 639 | 22.761 | 11.449 | 0.826 | 1.00 | 50.68 | A | N |
| ATOM | 646 | CE1 | HIS | A | 639 | 23.765 | 11.821 | 0.051 | 1.00 | 51.18 | A | C |
| ATOM | 647 | NE2 | HIS | A | 639 | 23.328 | 12.751 | −0.779 | 1.00 | 51.79 | A | N |
| ATOM | 648 | C | HIS | A | 639 | 21.291 | 13.460 | 2.937 | 1.00 | 39.66 | A | C |
| ATOM | 649 | O | HIS | A | 639 | 22.494 | 13.282 | 3.135 | 1.00 | 38.40 | A | C |
| ATOM | 650 | N | LEU | A | 640 | 20.735 | 14.666 | 2.915 | 1.00 | 37.63 | A | N |
| ATOM | 651 | CA | LEU | A | 640 | 21.525 | 15.874 | 3.114 | 1.00 | 37.30 | A | C |
| ATOM | 652 | CB | LEU | A | 640 | 20.668 | 17.122 | 2.873 | 1.00 | 37.29 | A | C |
| ATOM | 653 | CG | LEU | A | 640 | 20.243 | 17.467 | 1.445 | 1.00 | 39.00 | A | C |
| ATOM | 654 | CD1 | LEU | A | 640 | 19.407 | 18.743 | 1.465 | 1.00 | 39.56 | A | C |
| ATOM | 655 | CD2 | LEU | A | 640 | 21.467 | 17.650 | 0.570 | 1.00 | 38.33 | A | C |
| ATOM | 656 | C | LEU | A | 640 | 22.166 | 15.989 | 4.486 | 1.00 | 36.52 | A | C |
| ATOM | 657 | O | LEU | A | 640 | 23.313 | 16.415 | 4.603 | 1.00 | 37.87 | A | O |
| ATOM | 658 | N | GLY | A | 641 | 21.424 | 15.617 | 5.521 | 1.00 | 36.87 | A | N |
| ATOM | 659 | CA | GLY | A | 641 | 21.941 | 15.736 | 6.871 | 1.00 | 36.66 | A | C |
| ATOM | 660 | C | GLY | A | 641 | 21.496 | 17.081 | 7.414 | 1.00 | 36.66 | A | C |
| ATOM | 661 | O | GLY | A | 641 | 20.992 | 17.915 | 6.660 | 1.00 | 34.68 | A | O |
| ATOM | 662 | N | GLN | A | 642 | 21.694 | 17.312 | 8.707 | 1.00 | 35.84 | A | N |
| ATOM | 663 | CA | GLN | A | 642 | 21.265 | 18.564 | 9.321 | 1.00 | 36.94 | A | C |
| ATOM | 664 | CB | GLN | A | 642 | 20.971 | 18.341 | 10.811 | 1.00 | 39.69 | A | C |
| ATOM | 665 | CG | GLN | A | 642 | 19.797 | 17.411 | 11.089 | 1.00 | 46.06 | A | C |
| ATOM | 666 | CD | GLN | A | 642 | 19.281 | 17.517 | 12.524 | 1.00 | 50.41 | A | C |
| ATOM | 667 | OE1 | GLN | A | 642 | 18.807 | 18.573 | 12.951 | 1.00 | 52.43 | A | O |
| ATOM | 668 | NE2 | GLN | A | 642 | 19.371 | 16.421 | 13.271 | 1.00 | 52.58 | A | N |
| ATOM | 669 | C | GLN | A | 642 | 22.229 | 19.742 | 9.172 | 1.00 | 34.61 | A | C |
| ATOM | 670 | O | GLN | A | 642 | 23.433 | 19.564 | 9.011 | 1.00 | 34.08 | A | O |
| ATOM | 671 | N | HIS | A | 643 | 21.668 | 20.948 | 9.214 | 1.00 | 32.05 | A | N |
| ATOM | 672 | CA | HIS | A | 643 | 22.442 | 22.178 | 9.138 | 1.00 | 30.15 | A | C |
| ATOM | 673 | CB | HIS | A | 643 | 22.759 | 22.558 | 7.691 | 1.00 | 30.12 | A | C |
| ATOM | 674 | CG | HIS | A | 643 | 23.701 | 23.715 | 7.571 | 1.00 | 28.97 | A | C |
| ATOM | 675 | CD2 | HIS | A | 643 | 25.023 | 23.763 | 7.279 | 1.00 | 28.03 | A | C |
| ATOM | 676 | ND1 | HIS | A | 643 | 23.317 | 25.015 | 7.817 | 1.00 | 28.53 | A | N |
| ATOM | 677 | CE1 | HIS | A | 643 | 24.362 | 25.814 | 7.683 | 1.00 | 28.66 | A | C |
| ATOM | 678 | NE2 | HIS | A | 643 | 25.409 | 25.078 | 7.357 | 1.00 | 27.92 | A | N |
| ATOM | 679 | C | HIS | A | 643 | 21.617 | 23.267 | 9.807 | 1.00 | 28.97 | A | C |
| ATOM | 680 | O | HIS | A | 643 | 20.396 | 23.329 | 9.639 | 1.00 | 27.72 | A | O |
| ATOM | 681 | N | GLU | A | 644 | 22.285 | 24.124 | 10.570 | 1.00 | 28.26 | A | N |
| ATOM | 682 | CA | GLU | A | 644 | 21.606 | 25.177 | 11.303 | 1.00 | 28.55 | A | C |
| ATOM | 683 | CB | GLU | A | 644 | 22.625 | 25.979 | 12.125 | 1.00 | 32.74 | A | C |
| ATOM | 684 | CG | GLU | A | 644 | 23.890 | 26.354 | 11.375 | 1.00 | 38.99 | A | C |
| ATOM | 685 | CD | GLU | A | 644 | 25.014 | 25.341 | 11.558 | 1.00 | 42.01 | A | C |
| ATOM | 686 | OE1 | GLU | A | 644 | 24.945 | 24.228 | 10.986 | 1.00 | 42.31 | A | O |
| ATOM | 687 | OE2 | GLU | A | 644 | 25.976 | 25.666 | 12.289 | 1.00 | 45.83 | A | O |
| ATOM | 688 | C | GLU | A | 644 | 20.747 | 26.130 | 10.473 | 1.00 | 25.79 | A | C |
| ATOM | 689 | O | GLU | A | 644 | 19.773 | 26.678 | 10.976 | 1.00 | 25.50 | A | O |
| ATOM | 690 | N | ASN | A | 645 | 21.085 | 26.317 | 9.201 | 1.00 | 25.71 | A | N |
| ATOM | 691 | CA | ASN | A | 645 | 20.312 | 27.233 | 8.364 | 1.00 | 24.49 | A | C |
| ATOM | 692 | CB | ASN | A | 645 | 21.253 | 28.223 | 7.685 | 1.00 | 23.04 | A | C |
| ATOM | 693 | CG | ASN | A | 645 | 22.066 | 29.020 | 8.685 | 1.00 | 24.17 | A | C |
| ATOM | 694 | OD1 | ASN | A | 645 | 23.286 | 28.877 | 8.761 | 1.00 | 25.55 | A | O |
| ATOM | 695 | ND2 | ASN | A | 645 | 21.390 | 29.853 | 9.468 | 1.00 | 23.90 | A | N |
| ATOM | 696 | C | ASN | A | 645 | 19.425 | 26.545 | 7.322 | 1.00 | 25.98 | A | C |
| ATOM | 697 | O | ASN | A | 645 | 19.097 | 27.126 | 6.286 | 1.00 | 23.32 | A | O |
| ATOM | 698 | N | ILE | A | 646 | 19.053 | 25.305 | 7.609 | 1.00 | 26.04 | A | N |
| ATOM | 699 | CA | ILE | A | 646 | 18.162 | 24.544 | 6.754 | 1.00 | 26.70 | A | C |
| ATOM | 700 | CB | ILE | A | 646 | 18.841 | 23.250 | 6.246 | 1.00 | 28.88 | A | C |
| ATOM | 701 | CG2 | ILE | A | 646 | 17.830 | 22.370 | 5.531 | 1.00 | 31.04 | A | C |
| ATOM | 702 | CG1 | ILE | A | 646 | 20.021 | 23.594 | 5.327 | 1.00 | 31.04 | A | C |
| ATOM | 703 | CD1 | ILE | A | 646 | 19.635 | 24.278 | 4.045 | 1.00 | 34.16 | A | C |
| ATOM | 704 | C | ILE | A | 646 | 16.949 | 24.178 | 7.603 | 1.00 | 26.93 | A | C |
| ATOM | 705 | O | ILE | A | 646 | 17.062 | 24.025 | 8.827 | 1.00 | 27.80 | A | O |
| ATOM | 706 | N | VAL | A | 647 | 15.784 | 24.076 | 6.973 | 1.00 | 25.95 | A | N |
| ATOM | 707 | CA | VAL | A | 647 | 14.580 | 23.663 | 7.687 | 1.00 | 27.82 | A | C |
| ATOM | 708 | CB | VAL | A | 647 | 13.312 | 24.063 | 6.925 | 1.00 | 27.74 | A | C |
| ATOM | 709 | CG1 | VAL | A | 647 | 12.077 | 23.579 | 7.672 | 1.00 | 29.09 | A | C |
| ATOM | 710 | CG2 | VAL | A | 647 | 13.272 | 25.581 | 6.773 | 1.00 | 27.59 | A | C |
| ATOM | 711 | C | VAL | A | 647 | 14.739 | 22.144 | 7.704 | 1.00 | 28.05 | A | C |
| ATOM | 712 | O | VAL | A | 647 | 14.341 | 21.449 | 6.769 | 1.00 | 29.26 | A | O |
| ATOM | 713 | N | ASN | A | 648 | 15.347 | 21.645 | 8.775 | 1.00 | 28.15 | A | N |
| ATOM | 714 | CA | ASN | A | 648 | 15.653 | 20.227 | 8.931 | 1.00 | 26.26 | A | C |
| ATOM | 715 | CB | ASN | A | 648 | 16.573 | 20.033 | 10.145 | 1.00 | 26.67 | A | C |
| ATOM | 716 | CG | ASN | A | 648 | 17.902 | 20.734 | 9.989 | 1.00 | 26.26 | A | C |
| ATOM | 717 | OD1 | ASN | A | 648 | 18.714 | 20.379 | 9.135 | 1.00 | 26.67 | A | O |
| ATOM | 718 | ND2 | ASN | A | 648 | 18.130 | 21.747 | 10.813 | 1.00 | 28.60 | A | N |
| ATOM | 719 | C | ASN | A | 648 | 14.518 | 19.227 | 9.041 | 1.00 | 25.68 | A | C |
| ATOM | 720 | O | ASN | A | 648 | 13.458 | 19.505 | 9.598 | 1.00 | 26.37 | A | O |
| ATOM | 721 | N | LEU | A | 649 | 14.769 | 18.042 | 8.503 | 1.00 | 26.85 | A | N |
| ATOM | 722 | CA | LEU | A | 649 | 13.807 | 16.950 | 8.56 | 91.00 | 26.88 | A | C |
| ATOM | 723 | CB | LEU | A | 649 | 14.208 | 15.843 | 7.598 | 1.00 | 27.35 | A | C |
| ATOM | 724 | CG | LEU | A | 649 | 13.355 | 14.576 | 7.642 | 1.00 | 27.47 | A | C |
| ATOM | 725 | CD1 | LEU | A | 649 | 11.951 | 14.874 | 7.133 | 1.00 | 26.19 | A | C |
| ATOM | 726 | CD2 | LEU | A | 649 | 14.018 | 13.497 | 6.810 | 1.00 | 26.76 | A | C |
| ATOM | 727 | C | LEU | A | 649 | 13.873 | 16.414 | 10.002 | 1.00 | 28.37 | A | C |
| ATOM | 728 | O | LEU | A | 649 | 14.962 | 16.280 | 10.559 | 1.00 | 26.93 | A | O |
| ATOM | 729 | N | LEU | A | 650 | 12.721 | 16.119 | 10.595 | 1.00 | 28.89 | A | N |
| ATOM | 730 | CA | LEU | A | 650 | 12.680 | 15.603 | 11.961 | 1.00 | 33.13 | A | C |
| ATOM | 731 | CB | LEU | A | 650 | 11.940 | 16.585 | 12.870 | 1.00 | 33.70 | A | C |
| ATOM | 732 | CG | LEU | A | 650 | 12.580 | 17.970 | 12.987 | 1.00 | 34.96 | A | C |
| ATOM | 733 | CD1 | LEU | A | 650 | 11.746 | 18.843 | 13.910 | 1.00 | 35.56 | A | C |
| ATOM | 734 | CD2 | LEU | A | 650 | 14.004 | 17.833 | 13.511 | 1.00 | 35.61 | A | C |
| ATOM | 735 | C | LEU | A | 650 | 12.003 | 14.234 | 12.032 | 1.00 | 33.70 | A | C |
| ATOM | 736 | O | LEU | A | 650 | 12.216 | 13.469 | 12.979 | 1.00 | 33.23 | A | O |
| ATOM | 737 | N | GLY | A | 651 | 11.187 | 13.936 | 11.027 | 1.00 | 32.99 | A | N |
| ATOM | 738 | CA | GLY | A | 651 | 10.494 | 12.662 | 10.995 | 1.00 | 31.78 | A | C |
| ATOM | 739 | C | GLY | A | 651 | 9.522 | 12.551 | 9.842 | 1.00 | 30.54 | A | C |
| ATOM | 740 | O | GLY | A | 651 | 9.384 | 13.476 | 9.041 | 1.00 | 28.69 | A | O |
| ATOM | 741 | N | ALA | A | 652 | 8.841 | 11.412 | 9.761 | 1.00 | 29.49 | A | N |
| ATOM | 742 | CA | ALA | A | 652 | 7.873 | 11.172 | 8.705 | 1.00 | 29.18 | A | C |
| ATOM | 743 | CB | ALA | A | 652 | 8.589 | 10.764 | 7.435 | 1.00 | 28.96 | A | C |
| ATOM | 744 | C | ALA | A | 652 | 6.884 | 10.083 | 9.101 | 1.00 | 30.64 | A | C |
| ATOM | 745 | O | ALA | A | 652 | 7.180 | 9.231 | 9.940 | 1.00 | 30.25 | A | O |
| ATOM | 746 | N | CYS | A | 653 | 5.706 | 10.123 | 8.494 | 1.00 | 30.79 | A | N |
| ATOM | 747 | CA | CYS | A | 653 | 4.682 | 9.114 | 8.736 | 1.00 | 32.84 | A | C |
| ATOM | 748 | CB | CYS | A | 653 | 3.454 | 9.739 | 9.385 | 1.00 | 32.79 | A | C |
| ATOM | 749 | SG | CYS | A | 653 | 3.856 | 10.592 | 10.905 | 1.00 | 34.89 | A | S |
| ATOM | 750 | C | CYS | A | 653 | 4.344 | 8.579 | 7.357 | 1.00 | 33.55 | A | C |
| ATOM | 751 | O | CYS | A | 653 | 3.656 | 9.237 | 6.574 | 1.00 | 33.19 | A | O |
| ATOM | 752 | N | THR | A | 654 | 4.843 | 7.391 | 7.044. | 1.00 | 33.09 | A | N |
| ATOM | 753 | CA | THR | A | 654 | 4.606 | 6.830 | 5.722 | 1.00 | 33.59 | A | C |
| ATOM | 754 | CB | THR | A | 654 | 5.938 | 6.526 | 5.023 | 1.00 | 33.05 | A | C |
| ATOM | 755 | OG1 | THR | A | 654 | 6.655 | 5.533 | 5.767 | 1.00 | 32.61 | A | O |
| ATOM | 756 | CG2 | THR | A | 654 | 6.788 | 7.793 | 4.945 | 1.00 | 33.62 | A | C |
| ATOM | 757 | C | THR | A | 654 | 3.743 | 5.579 | 5.726 | 1.00 | 35.09 | A | C |
| ATOM | 758 | O | THR | A | 654 | 3.459 | 5.021 | 4.669 | 1.00 | 34.98 | A | O |
| ATOM | 759 | N | HIS | A | 655 | 3.322 | 5.152 | 6.915 | 1.00 | 35.32 | A | N |
| ATOM | 760 | CA | HIS | A | 655 | 2.473 | 3.974 | 7.049 | 1.00 | 36.55 | A | C |
| ATOM | 761 | CB | HIS | A | 655 | 3.235 | 2.838 | 7.744 | 1.00 | 36.90 | A | C |
| ATOM | 762 | CG | HIS | A | 655 | 4.554 | 2.519 | 7.113 | 1.00 | 38.59 | A | C |
| ATOM | 763 | CD2 | HIS | A | 655 | 5.818 | 2.626 | 7.587 | 1.00 | 37.83 | A | C |
| ATOM | 764 | ND1 | HIS | A | 655 | 4.669 | 2.045 | 5.823 | 1.00 | 39.61 | A | N |
| ATOM | 765 | CE1 | HIS | A | 655 | 5.946 | 1.875 | 5.531 | 1.00 | 38.84 | A | C |
| ATOM | 766 | NE2 | HIS | A | 655 | 6.664 | 2.221 | 6.584 | 1.00 | 37.56 | A | N |
| ATOM | 767 | C | HIS | A | 655 | 1.234 | 4.324 | 7.863 | 1.00 | 36.37 | A | C |
| ATOM | 768 | O | HIS | A | 655 | 1.229 | 5.297 | 8.618 | 1.00 | 38.33 | A | O |
| ATOM | 769 | N | GLY | A | 656 | 0.182 | 3.530 | 7.695 | 1.00 | 36.82 | A | N |
| ATOM | 770 | CA | GLY | A | 656 | −1.047 | 3.746 | 8.434 | 1.00 | 36.86 | A | C |
| ATOM | 771 | C | GLY | A | 656 | −1.819 | 4.995 | 8.069 | 1.00 | 36.39 | A | C |
| ATOM | 772 | O | GLY | A | 656 | −2.604 | 5.492 | 8.876 | 1.00 | 38.07 | A | O |
| ATOM | 773 | N | GLY | A | 657 | −1.604 | 5.503 | 6.858 | 1.00 | 36.26 | A | N |
| ATOM | 774 | CA | GLY | A | 657 | −2.303 | 6.702 | 6.429 | 1.00 | 36.55 | A | C |
| ATOM | 775 | C | GLY | A | 657 | −1.517 | 7.525 | 5.424 | 1.00 | 35.98 | A | C |
| ATOM | 776 | O | GLY | A | 657 | −0.442 | 7.113 | 4.995 | 1.00 | 35.12 | A | O |
| ATOM | 777 | N | PRO | A | 658 | −2.035 | 8.696 | 5.018 | 1.00 | 36.54 | A | N |
| ATOM | 778 | CD | PRO | A | 658 | −3.343 | 9.268 | 5.384 | 1.00 | 38.10 | A | C |
| ATOM | 779 | CA | PRO | A | 658 | −1.343 | 9.556 | 4.052 | 1.00 | 36.84 | A | C |
| ATOM | 780 | CB | PRO | A | 658 | −2.226 | 10.804 | 4.012 | 1.00 | 36.89 | A | C |
| ATOM | 781 | CG | PRO | A | 658 | −3.591 | 10.248 | 4.261 | 1.00 | 39.35 | A | C |
| ATOM | 782 | C | PRO | A | 658 | 0.084 | 9.875 | 4.502 | 1.00 | 32.89 | A | C |
| ATOM | 783 | O | PRO | A | 658 | 0.358 | 9.970 | 5.696 | 1.00 | 31.66 | A | O |
| ATOM | 784 | N | VAL | A | 659 | 0.989 | 10.036 | 3.543 | 1.00 | 32.62 | A | N |
| ATOM | 785 | CA | VAL | A | 659 | 2.379 | 10.354 | 3.858 | 1.00 | 31.70 | A | C |
| ATOM | 786 | CB | VAL | A | 659 | 3.266 | 10.282 | 2.591 | 1.00 | 31.79 | A | C |
| ATOM | 787 | CG1 | VAL | A | 659 | 4.672 | 10.807 | 2.891 | 1.00 | 31.41 | A | C |
| ATOM | 788 | CG2 | VAL | A | 659 | 3.339 | 8.845 | 2.100 | 1.00 | 32.68 | A | C |
| ATOM | 789 | C | VAL | A | 659 | 2.496 | 11.750 | 4.467 | 1.00 | 31.56 | A | C |
| ATOM | 790 | O | VAL | A | 659 | 1.960 | 12.724 | 3.926 | 1.00 | 32.04 | A | O |
| ATOM | 791 | N | LEU | A | 660 | 3.186 | 11.833 | 5.598 | 1.00 | 28.87 | A | N |
| ATOM | 792 | CA | LEU | A | 660 | 3.393 | 13.100 | 6.291 | 1.00 | 29.74 | A | C |
| ATOM | 793 | CB | LEU | A | 660 | 2.722 | 13.076 | 7.667 | 1.00 | 30.94 | A | C |
| ATOM | 794 | CG | LEU | A | 660 | 1.249 | 12.672 | 7.770 | 1.00 | 31.84 | A | C |
| ATOM | 795 | CD1 | LEU | A | 660 | 0.821 | 12.721 | 9.230 | 1.00 | 33.18 | A | C |
| ATOM | 796 | CD2 | LEU | A | 660 | 0.388 | 13.601 | 6.929 | 1.00 | 33.70 | A | C |
| ATOM | 797 | C | LEU | A | 660 | 4.885 | 13.344 | 6.486 | 1.00 | 28.75 | A | C |
| ATOM | 798 | O | LEU | A | 660 | 5.609 | 12.457 | 6.942 | 1.00 | 28.50 | A | O |
| ATOM | 799 | N | VAL | A | 661 | 5.349 | 14.543 | 6.148 | 1.00 | 28.59 | A | N |
| ATOM | 800 | CA | VAL | A | 661 | 6.760 | 14.872 | 6.329 | 1.00 | 27.56 | A | C |
| ATOM | 801 | CB | VAL | A | 661 | 7.391 | 15.394 | 5.030 | 1.00 | 29.16 | A | C |
| ATOM | 802 | CG1 | VAL | A | 661 | 8.879 | 15.670 | 5.253 | 1.00 | 30.68 | A | C |
| ATOM | 803 | CG2 | VAL | A | 661 | 7.189 | 14.372 | 3.910 | 1.00 | 26.50 | A | C |
| ATOM | 804 | C | VAL | A | 661 | 6.882 | 15.927 | 7.418 | 1.00 | 29.07 | A | C |
| ATOM | 805 | O | VAL | A | 661 | 6.383 | 17.043 | 7.273 | 1.00 | 29.29 | A | O |
| ATOM | 806 | N | ILE | A | 662 | 7.555 | 15.565 | 8.506 | 1.00 | 27.78 | A | N |
| ATOM | 807 | CA | ILE | A | 662 | 7.714. | 16.451 | 9.651 | 1.00 | 29.50 | A | C |
| ATOM | 808 | CB | ILE | A | 662 | 7.625 | 15.657 | 10.970 | 1.00 | 29.86 | A | C |
| ATOM | 809 | CG2 | ILE | A | 662 | 7.496 | 16.620 | 12.143 | 1.00 | 30.78 | A | C |
| ATOM | 810 | CG1 | ILE | A | 662 | 6.411 | 14.721 | 10.939 | 1.00 | 32.59 | A | C |
| ATOM | 811 | CD1 | ILE | A | 662 | 6.422 | 13.672 | 12.043 | 1.00 | 32.41 | A | C |
| ATOM | 812 | C | ILE | A | 662 | 9.043 | 17.188 | 9.630 | 1.00 | 28.44 | A | C |
| ATOM | 813 | O | ILE | A | 662 | 10.101 | 16.567 | 9.605 | 1.00 | 30.01 | A | O |
| ATOM | 814 | N | THR | A | 663 | 8.983 | 18.515 | 9.639 | 1.00 | 28.71 | A | N |
| ATOM | 815 | CA | THR | A | 663 | 10.196 | 19.329 | 9.619 | 1.00 | 28.93 | A | C |
| ATOM | 816 | CB | THR | A | 663 | 10.347 | 20.110 | 8.290 | 1.00 | 27.31 | A | C |
| ATOM | 817 | OG1 | THR | A | 663 | 9.352 | 21.140 | 8.230 | 1.00 | 29.33 | A | O |
| ATOM | 818 | CG2 | THR | A | 663 | 10.168 | 19.187 | 7.094 | 1.00 | 29.13 | A | C |
| ATOM | 819 | C | THR | A | 663 | 10.126 | 20.355 | 10.736 | 1.00 | 29.48 | A | C |
| ATOM | 820 | O | THR | A | 663 | 9.096 | 20.506 | 11.389 | 1.00 | 31.00 | A | O |
| ATOM | 821 | N | GLU | A | 664 | 11.225 | 21.064 | 10.957 | 1.00 | 29.67 | A | N |
| ATOM | 822 | CA | GLU | A | 664 | 11.241 | 22.097 | 11.981 | 1.00 | 31.74 | A | C |
| ATOM | 823 | CB | GLU | A | 664 | 12.630 | 22.729 | 12.075 | 1.00 | 31.41 | A | C |
| ATOM | 824 | CG | GLU | A | 664 | 13.736 | 21.729 | 12.333 | 1.00 | 34.94 | A | C |
| ATOM | 825 | CD | GLU | A | 664 | 15.112 | 22.371 | 12.341 | 1.00 | 37.50 | A | C |
| ATOM | 826 | OE1 | GLU | A | 664 | 15.423 | 23.132 | 11.398 | 1.00 | 36.63 | A | O |
| ATOM | 827 | OE2 | GLU | A | 664 | 15.885 | 22.107 | 13.284 | 1.00 | 39.81 | A | O |
| ATOM | 828 | C | GLU | A | 664 | 10.216 | 23.149 | 11.564 | 1.00 | 31.00 | A | C |
| ATOM | 829 | O | GLU | A | 664 | 9.918 | 23.300 | 10.374 | 1.00 | 28.32 | A | O |
| ATOM | 830 | N | TYR | A | 665 | 9.667 | 23.851 | 12.550 | 1.00 | 32.33 | A | N |
| ATOM | 831 | CA | TYR | A | 665 | 8.680 | 24.901 | 12.324 | 1.00 | 32.77 | A | C |
| ATOM | 832 | CB | TYR | A | 665 | 7.508 | 24.723 | 13.299 | 1.00 | 34.33 | A | C |
| ATOM | 833 | CG | TYR | A | 665 | 6.602 | 25.930 | 13.444 | 1.00 | 34.33 | A | C |
| ATOM | 834 | CD1 | TYR | A | 665 | 5.779 | 26.347 | 12.399 | 1.00 | 35.01 | A | C |
| ATOM | 835 | CE1 | TYR | A | 665 | 4.932 | 27.453 | 12.545 | 1.00 | 36.08 | A | C |
| ATOM | 836 | CD2 | TYR | A | 665 | 6.561 | 26.647 | 14.640 | 1.00 | 35.79 | A | C |
| ATOM | 837 | CE2 | TYR | A | 665 | 5.721 | 27.748 | 14.795 | 1.00 | 35.87 | A | C |
| ATOM | 838 | CZ | TYR | A | 665 | 4.910 | 28.146 | 13.748 | 1.00 | 37.04 | A | C |
| ATOM | 839 | OH | TYR | A | 665 | 4.078 | 29.230 | 13.911 | 1.00 | 38.04 | A | O |
| ATOM | 840 | C | TYR | A | 665 | 9.385 | 26.243 | 12.558 | 1.00 | 33.09 | A | C |
| ATOM | 841 | O | TYR | A | 665 | 10.188 | 26.369 | 13.479 | 1.00 | 32.45 | A | O |
| ATOM | 842 | N | CYS | A | 666 | 9.098 | 27.231 | 11.714 | 1.00 | 33.49 | A | N |
| ATOM | 843 | CA | CYS | A | 666 | 9.719 | 28.551 | 11.832 | 1.00 | 32.76 | A | C |
| ATOM | 844 | CB | CYS | A | 666 | 10.359 | 28.935 | 10.487 | 1.00 | 32.71 | A | C |
| ATOM | 845 | SG | CYS | A | 666 | 11.647 | 27.746 | 9.963 | 1.00 | 31.28 | A | S |
| ATOM | 846 | C | CYS | A | 666 | 8.664 | 29.572 | 12.256 | 1.00 | 33.47 | A | C |
| ATOM | 847 | O | CYS | A | 666 | 7.947 | 30.132 | 11.425 | 1.00 | 31.41 | A | O |
| ATOM | 848 | N | CYS | A | 667 | 8.591 | 29.810 | 13.565 | 1.00 | 34.90 | A | N |
| ATOM | 849 | CA | CYS | A | 667 | 7.598 | 30.705 | 14.155 | 1.00 | 36.88 | A | C |
| ATOM | 850 | CB | CYS | A | 667 | 7.831 | 30.809 | 15.671 | 1.00 | 38.94 | A | C |
| ATOM | 851 | SG | CYS | A | 667 | 9.405 | 31.544 | 16.162 | 1.00 | 44.69 | A | S |
| ATOM | 852 | C | CYS | A | 667 | 7.406 | 32.111 | 13.583 | 1.00 | 35.74 | A | C |
| ATOM | 853 | O | CYS | A | 667 | 6.293 | 32.628 | 13.615 | 1.00 | 36.27 | A | O |
| ATOM | 854 | N | TYR | A | 668 | 8.460 | 32.733 | 13.059 | 1.00 | 35.00 | A | N |
| ATOM | 855 | CA | TYR | A | 668 | 8.319 | 34.087 | 12.519 | 1.00 | 33.58 | A | C |
| ATOM | 856 | CB | TYR | A | 668 | 9.631 | 34.854 | 12.677 | 1.00 | 36.69 | A | C |
| ATOM | 857 | CG | TYR | A | 668 | 9.991 | 35.099 | 14.123 | 1.00 | 39.90 | A | C |
| ATOM | 858 | CD1 | TYR | A | 668 | 11.188 | 34.623 | 14.657 | 1.00 | 40.56 | A | C |
| ATOM | 859 | CE1 | TYR | A | 668 | 11.511 | 34.828 | 15.998 | 1.00 | 43.29 | A | C |
| ATOM | 860 | CD2 | TYR | A | 668 | 9.121 | 35.792 | 14.967 | 1.00 | 43.12 | A | C |
| ATOM | 861 | CE2 | TYR | A | 668 | 9.433 | 36.003 | 16.309 | 1.00 | 43.78 | A | C |
| ATOM | 862 | CZ | TYR | A | 668 | 10.628 | 35.517 | 16.816 | 1.00 | 44.47 | A | C |
| ATOM | 863 | OH | TYR | A | 668 | 10.930 | 35.707 | 18.142 | 1.00 | 47.51 | A | O |
| ATOM | 864 | C | TYR | A | 668 | 7.848 | 34.153 | 11.070 | 1.00 | 32.26 | A | C |
| ATOM | 865 | O | TYR | A | 668 | 7.569 | 35.232 | 10.546 | 1.00 | 31.85 | A | O |
| ATOM | 866 | N | GLY | A | 669 | 7.751 | 33.002 | 10.422 | 1.00 | 28.38 | A | N |
| ATOM | 867 | CA | GLY | A | 669 | 7.292 | 32.997 | 9.045 | 1.00 | 29.22 | A | C |
| ATOM | 868 | C | GLY | A | 669 | 8.368 | 33.395 | 8.054 | 1.00 | 26.58 | A | C |
| ATOM | 869 | O | GLY | A | 669 | 9.561 | 33.302 | 8.348 | 1.00 | 27.94 | A | O |
| ATOM | 870 | N | ASP | A | 670 | 7.945 | 33.860 | 6.885 | 1.00 | 27.59 | A | N |
| ATOM | 871 | CA | ASP | A | 670 | 8.882 | 34.232 | 5.829 | 1.00 | 26.96 | A | C |
| ATOM | 872 | CB | ASP | A | 670 | 8.182 | 34.171 | 4.469 | 1.00 | 27.09 | A | C |
| ATOM | 873 | CG | ASP | A | 670 | 7.106 | 35.223 | 4.316 | 1.00 | 29.74 | A | C |
| ATOM | 874 | OD1 | ASP | A | 670 | 6.078 | 35.145 | 5.024 | 1.00 | 30.78 | A | O |
| ATOM | 875 | OD2 | ASP | A | 670 | 7.290 | 36.137 | 3.487 | 1.00 | 29.75 | A | O |
| ATOM | 876 | C | ASP | A | 670 | 9.560 | 35.589 | 5.983 | 1.00 | 25.53 | A | C |
| ATOM | 877 | O | ASP | A | 670 | 8.982 | 36.541 | 6.512 | 1.00 | 24.94 | A | O |
| ATOM | 878 | N | LEU | A | 671 | 10.789 | 35.661 | 5.480 | 1.00 | 25.51 | A | N |
| ATOM | 879 | CA | LEU | A | 671 | 11.609 | 36.864 | 5.537 | 1.00 | 24.25 | A | C |
| ATOM | 880 | CB | LEU | A | 671 | 13.017 | 36.565 | 4.996 | 1.00 | 22.80 | A | C |
| ATOM | 881 | CG | LEU | A | 671 | 13.992 | 37.744 | 4.925 | 1.00 | 22.98 | A | C |
| ATOM | 882 | CD1 | LEU | A | 671 | 14.258 | 38.265 | 6.332 | 1.00 | 21.43 | A | C |
| ATOM | 883 | CD2 | LEU | A | 671 | 15.296 | 37.307 | 4.251 | 1.00 | 23.79 | A | C |
| ATOM | 884 | C | LEU | A | 671 | 11.015 | 38.041 | 4.774 | 1.00 | 25.39 | A | C |
| ATOM | 885 | O | LEU | A | 671 | 11.160 | 39.187 | 5.198 | 1.00 | 25.04 | A | O |
| ATOM | 886 | N | LEU | A | 672 | 10.364 | 37.766 | 3.647 | 1.00 | 23.35 | A | N |
| ATOM | 887 | CA | LEU | A | 672 | 9.763 | 38.837 | 2.856 | 1.00 | 25.62 | A | C |
| ATOM | 888 | CB | LEU | A | 672 | 9.126 | 38.277 | 1.583 | 1.00 | 22.70 | A | C |
| ATOM | 889 | CG | LEU | A | 672 | 8.513 | 39.348 | 0.674 | 1.00 | 22.06 | A | C |
| ATOM | 890 | CD1 | LEU | A | 672 | 9.570 | 40.431 | 0.362 | 1.00 | 23.78 | A | C |
| ATOM | 891 | CD2 | LEU | A | 672 | 8.016 | 38.707 | −0.598 | 1.00 | 23.53 | A | C |
| ATOM | 892 | C | LEU | A | 672 | 8.713 | 39.608 | 3.662 | 1.00 | 26.62 | A | C |
| ATOM | 893 | O | LEU | A | 672 | 8.737 | 40.841 | 3.707 | 1.00 | 27.62 | A | O |
| ATOM | 894 | N | ASN | A | 673 | 7.784 | 38.888 | 4.284 | 1.00 | 27.85 | A | N |
| ATOM | 895 | CA | ASN | A | 673 | 6.760 | 39.542 | 5.093 | 1.00 | 29.94 | A | C |
| ATOM | 896 | CB | ASN | A | 673 | 5.706 | 38.539 | 5.567 | 1.00 | 30.08 | A | C |
| ATOM | 897 | CG | ASN | A | 673 | 4.636 | 38.286 | 4.518 | 1.00 | 33.58 | A | C |
| ATOM | 898 | OD1 | ASN | A | 673 | 4.035 | 39.225 | 3.988 | 1.00 | 34.48 | A | O |
| ATOM | 899 | ND2 | ASN | A | 673 | 4.388 | 37.020 | 4.218 | 1.00 | 36.08 | A | N |
| ATOM | 900 | C | ASN | A | 673 | 7.383 | 40.247 | 6.291 | 1.00 | 28.90 | A | C |
| ATOM | 901 | O | ASN | A | 673 | 6.936 | 41.323 | 6.678 | 1.00 | 28.73 | A | O |
| ATOM | 902 | N | PHE | A | 674 | 8.417 | 39.644 | 6.869 | 1.00 | 29.72 | A | N |
| ATOM | 903 | CA | PHE | A | 674 | 9.115 | 40.223 | 8.019 | 1.00 | 30.74 | A | C |
| ATOM | 904 | CE | PHE | A | 674 | 10.205 | 39.263 | 8.507 | 1.00 | 31.80 | A | C |
| ATOM | 905 | CG | PHE | A | 674 | 10.980 | 39.772 | 9.687 | 1.00 | 33.56 | A | C |
| ATOM | 906 | CD1 | PHE | A | 674 | 10.437 | 39.724 | 10.968 | 1.00 | 36.14 | A | C |
| ATOM | 907 | CD2 | PHE | A | 674 | 12.254 | 40.312 | 9.519 | 1.00 | 34.84 | A | C |
| ATOM | 908 | CE1 | PHE | A | 674 | 11.151 | 40.208 | 12.065 | 1.00 | 35.06 | A | C |
| ATOM | 909 | CE2 | PHE | A | 674 | 12.976 | 40.797 | 10.608 | 1.00 | 34.87 | A | C |
| ATOM | 910 | CZ | PHE | A | 674 | 12.422 | 40.745 | 11.886 | 1.00 | 34.77 | A | C |
| ATOM | 911 | C | PHE | A | 674 | 9.745 | 41.571 | 7.668 | 1.00 | 32.95 | A | C |
| ATOM | 912 | O | PHE | A | 674 | 9.679 | 42.525 | 8.449 | 1.00 | 32.49 | A | O |
| ATOM | 913 | N | LEU | A | 675 | 10.368 | 41.644 | 6.494 | 1.00 | 32.44 | A | N |
| ATOM | 914 | CA | LEU | A | 675 | 11.003 | 42.874 | 6.038 | 1.00 | 34.31 | A | C |
| ATOM | 915 | CE | LEU | A | 675 | 11.814 | 42.615 | 4.758 | 1.00 | 32.66 | A | C |
| ATOM | 916 | CG | LEU | A | 675 | 13.022 | 41.692 | 4.909 | 1.00 | 32.78 | A | C |
| ATOM | 917 | CD1 | LEU | A | 675 | 13.614 | 41.373 | 3.533 | 1.00 | 30.78 | A | C |
| ATOM | 918 | CD2 | LEU | A | 675 | 14.054 | 42.363 | 5.804 | 1.00 | 35.05 | A | C |
| ATOM | 919 | C | LEU | A | 675 | 9.968 | 43.955 | 5.764 | 1.00 | 34.32 | A | C |
| ATOM | 920 | O | LEU | A | 675 | 10.199 | 45.131 | 6.040 | 1.00 | 35.27 | A | O |
| ATOM | 921 | N | ARG | A | 676 | 8.825 | 43.553 | 5.220 | 1.00 | 35.94 | A | N |
| ATOM | 922 | CA | ARG | A | 676 | 7.768 | 44.500 | 4.898 | 1.00 | 39.37 | A | C |
| ATOM | 923 | CB | ARG | A | 676 | 6.744 | 43.832 | 3.976 | 1.00 | 37.16 | A | C |
| ATOM | 924 | CG | ARG | A | 676 | 7.274 | 43.717 | 2.544 | 1.00 | 34.09 | A | C |
| ATOM | 925 | CD | ARG | A | 676 | 6.413 | 42.878 | 1.617 | 1.00 | 34.64 | A | C |
| ATOM | 926 | NE | ARG | A | 676 | 6.988 | 42.888 | 0.273 | 1.00 | 31.11 | A | N |
| ATOM | 927 | CZ | ARG | A | 676 | 6.408 | 42.380 | −0.809 | 1.00 | 31.83 | A | C |
| ATOM | 928 | NH1 | ARG | A | 676 | 5.219 | 41.802 | −0.723 | 1.00 | 32.24 | A | N |
| ATOM | 929 | NH2 | ARG | A | 676 | 7.012 | 42.479 | −1.987 | 1.00 | 30.68 | A | N |
| ATOM | 930 | C | ARG | A | 676 | 7.092 | 45.120 | 6.123 | 1.00 | 43.44 | A | C |
| ATOM | 931 | O | ARG | A | 676 | 6.626 | 46.257 | 6.067 | 1.00 | 43.79 | A | O |
| ATOM | 932 | N | ARG | A | 677 | 7.058 | 44.392 | 7.233 | 1.00 | 46.91 | A | N |
| ATOM | 933 | CA | ARG | A | 677 | 6.445 | 44.918 | 8.447 | 1.00 | 52.48 | A | C |
| ATOM | 934 | CE | ARG | A | 677 | 5.751 | 43.791 | 9.214 | 1.00 | 54.41 | A | C |
| ATOM | 935 | CG | ARG | A | 677 | 6.675 | 42.671 | 9.650 | 1.00 | 58.12 | A | C |
| ATOM | 936 | CD | ARG | A | 677 | 5.886 | 41.446 | 10.082 | 1.00 | 61.40 | A | C |
| ATOM | 937 | NE | ARG | A | 677 | 6.764 | 40.350 | 10.484 | 1.00 | 64.57 | A | N |
| ATOM | 938 | CZ | ARG | A | 677 | 6.368 | 39.091 | 10.625 | 1.00 | 65.16 | A | C |
| ATOM | 939 | NH1 | ARG | A | 677 | 5.104 | 38.764 | 10.393 | 1.00 | 66.95 | A | N |
| ATOM | 940 | NH2 | ARG | A | 677 | 7.234 | 38.159 | 11.000 | 1.00 | 66.24 | A | N |
| ATOM | 941 | C | ARG | A | 677 | 7.485 | 45.604 | 9.335 | 1.00 | 54.60 | A | C |
| ATOM | 942 | O | ARG | A | 677 | 7.156 | 46.135 | 10.394 | 1.00 | 55.10 | A | O |
| ATOM | 943 | N | LYS | A | 678 | 8.739 | 45.599 | 8.887 | 1.00 | 56.59 | A | N |
| ATOM | 944 | CA | LYS | A | 678 | 9.832 | 46.217 | 9.631 | 1.00 | 58.57 | A | C |
| ATOM | 945 | CE | LYS | A | 678 | 10.987 | 45.225 | 9.793 | 1.00 | 58.89 | A | C |
| ATOM | 946 | CG | LYS | A | 678 | 10.839 | 44.269 | 10.964 | 1.00 | 59.02 | A | C |
| ATOM | 947 | CD | LYS | A | 678 | 10.957 | 45.010 | 12.282 | 1.00 | 59.46 | A | C |
| ATOM | 948 | CE | LYS | A | 678 | 10.929 | 44.056 | 13.471 | 1.00 | 59.83 | A | C |
| ATOM | 949 | NZ | LYS | A | 678 | 9.635 | 43.324 | 13.576 | 1.00 | 60.21 | A | N |
| ATOM | 950 | C | LYS | A | 678 | 10.357 | 47.495 | 8.980 | 1.00 | 59.55 | A | C |
| ATOM | 951 | O | LYS | A | 678 | 11.309 | 48.099 | 9.472 | 1.00 | 60.50 | A | O |
| ATOM | 952 | N | ARG | A | 679 | 9.741 | 47.904 | 7.877 | 1.00 | 60.41 | A | N |
| ATOM | 953 | CA | ARG | A | 679 | 10.167 | 49.113 | 7.179 | 1.00 | 61.24 | A | C |
| ATOM | 954 | CB | ARG | A | 679 | 9.368 | 49.287 | 5.885 | 1.00 | 61.34 | A | C |
| ATOM | 955 | CG | ARG | A | 679 | 9.603 | 48.191 | 4.858 | 1.00 | 61.16 | A | C |
| ATOM | 956 | CD | ARG | A | 679 | 8.858 | 48.477 | 3.568 | 1.00 | 61.28 | A | C |
| ATOM | 957 | NE | ARG | A | 679 | 9.057 | 47.423 | 2.578 | 1.00 | 61.37 | A | N |
| ATOM | 958 | CZ | ARG | A | 679 | 8.497 | 47.413 | 1.373 | 1.00 | 61.59 | A | C |
| ATOM | 959 | NH1 | ARG | A | 679 | 7.698 | 48.403 | 0.999 | 1.00 | 62.21 | A | N |
| ATOM | 960 | NH2 | ARG | A | 679 | 8.736 | 46.411 | 0.540 | 1.00 | 61.00 | A | N |
| ATOM | 961 | C | ARG | A | 679 | 10.007 | 50.357 | 8.052 | 1.00 | 61.79 | A | C |
| ATOM | 962 | O | ARG | A | 679 | 9.226 | 50.367 | 9.005 | 1.00 | 62.55 | A | O |
| ATOM | 963 | N | GLN | A | 696 | 12.785 | 47.275 | 15.629 | 1.00 | 61.78 | A | N |
| ATOM | 964 | CA | GLN | A | 696 | 13.294 | 47.782 | 14.360 | 1.00 | 60.72 | A | C |
| ATOM | 965 | CB | GLN | A | 696 | 13.701 | 49.250 | 14.502 | 1.00 | 62.22 | A | C |
| ATOM | 966 | CG | GLN | A | 696 | 12.622 | 50.245 | 14.088 | 1.00 | 64.62 | A | C |
| ATOM | 967 | CD | GLN | A | 696 | 12.320 | 50.192 | 12.598 | 1.00 | 65.58 | A | C |
| ATOM | 968 | OE1 | GLN | A | 696 | 11.817 | 49.189 | 12.089 | 1.00 | 66.39 | A | O |
| ATOM | 969 | NE2 | GLN | A | 696 | 12.632 | 51.273 | 11.891 | 1.00 | 66.10 | A | N |
| ATOM | 970 | C | GLN | A | 696 | 14.480 | 46.980 | 13.843 | 1.00 | 58.89 | A | C |
| ATOM | 971 | O | GLN | A | 696 | 15.215 | 46.369 | 14.617 | 1.00 | 58.81 | A | O |
| ATOM | 972 | N | LEU | A | 697 | 14.656 | 46.987 | 12.525 | 1.00 | 56.74 | A | N |
| ATOM | 973 | CA | LEU | A | 697 | 15.756 | 46.270 | 11.893 | 1.00 | 54.36 | A | C |
| ATOM | 974 | CB | LEU | A | 697 | 15.348 | 45.766 | 10.505 | 1.00 | 55.20 | A | C |
| ATOM | 975 | CG | LEU | A | 697 | 14.526 | 44.477 | 10.439 | 1.00 | 55.34 | A | C |
| ATOM | 976 | CD1 | LEU | A | 697 | 14.125 | 44.196 | 9.000 | 1.00 | 54.88 | A | C |
| ATOM | 977 | CD2 | LEU | A | 697 | 15.346 | 43.326 | 11.002 | 1.00 | 55.01 | A | C |
| ATOM | 978 | C | LEU | A | 697 | 16.985 | 47.150 | 11.758 | 1.00 | 52.28 | A | C |
| ATOM | 979 | O | LEU | A | 697 | 16.882 | 48.337 | 11.451 | 1.00 | 53.04 | A | O |
| ATOM | 980 | N | SER | A | 698 | 18.150 | 46.559 | 11.985 | 1.00 | 48.72 | A | N |
| ATOM | 981 | CA | SER | A | 698 | 19.402 | 47.286 | 11.873 | 1.00 | 45.53 | A | C |
| ATOM | 982 | CB | SER | A | 698 | 20.250 | 47.077 | 13.124 | 1.00 | 45.79 | A | C |
| ATOM | 983 | OG | SER | A | 698 | 20.717 | 45.743 | 13.182 | 1.00 | 43.87 | A | O |
| ATOM | 984 | C | SER | A | 698 | 20.151 | 46.749 | 10.667 | 1.00 | 44.43 | A | C |
| ATOM | 985 | O | SER | A | 698 | 19.780 | 45.716 | 10.108 | 1.00 | 42.52 | A | O |
| ATOM | 986 | N | SER | A | 699 | 21.208 | 47.451 | 10.272 | 1.00 | 43.32 | A | N |
| ATOM | 987 | CA | SER | A | 699 | 22.020 | 47.025 | 9.144 | 1.00 | 43.23 | A | C |
| ATOM | 988 | CB | SER | A | 699 | 23.090 | 48.081 | 8.831 | 1.00 | 45.00 | A | C |
| ATOM | 989 | OG | SER | A | 699 | 23.855 | 48.400 | 9.982 | 1.00 | 49.17 | A | O |
| ATOM | 990 | C | SER | A | 699 | 22.664 | 45.685 | 9.492 | 1.00 | 42.93 | A | C |
| ATOM | 991 | O | SER | A | 699 | 22.818 | 44.816 | 8.636 | 1.00 | 40.20 | A | O |
| ATOM | 992 | N | ARG | A | 753 | 23.022 | 45.521 | 10.760 | 1.00 | 41.17 | A | N |
| ATOM | 993 | CA | ARG | A | 753 | 23.637 | 44.283 | 11.227 | 1.00 | 41.41 | A | C |
| ATOM | 994 | CB | ARG | A | 753 | 24.010 | 44.409 | 12.710 | 1.00 | 44.31 | A | C |
| ATOM | 995 | CG | ARG | A | 753 | 24.714 | 43.192 | 13.286 | 1.00 | 48.67 | A | C |
| ATOM | 996 | CD | ARG | A | 753 | 25.323 | 43.494 | 14.649 | 1.00 | 53.14 | A | C |
| ATOM | 997 | NE | ARG | A | 753 | 26.362 | 44.517 | 14.562 | 1.00 | 56.83 | A | N |
| ATOM | 998 | CZ | ARG | A | 753 | 27.115 | 44.912 | 15.585 | 1.00 | 59.04 | A | C |
| ATOM | 999 | NH1 | ARG | A | 753 | 26.950 | 44.369 | 16.785 | 1.00 | 59.71 | A | N |
| ATOM | 1000 | NH2 | ARG | A | 753 | 28.033 | 45.853 | 15.408 | 1.00 | 60.73 | A | N |
| ATOM | 1001 | C | ARG | A | 753 | 22.690 | 43.095 | 11.017 | 1.00 | 38.44 | A | C |
| ATOM | 1002 | O | ARG | A | 753 | 23.124 | 42.011 | 10.618 | 1.00 | 38.12 | A | O |
| ATOM | 1003 | N | ASP | A | 754 | 21.402 | 43.302 | 11.288 | 1.00 | 35.66 | A | N |
| ATOM | 1004 | CA | ASP | A | 754 | 20.397 | 42.256 | 11.112 | 1.00 | 33.99 | A | C |
| ATOM | 1005 | CB | ASP | A | 754 | 19.013 | 42.762 | 11.525 | 1.00 | 36.68 | A | C |
| ATOM | 1006 | CG | ASP | A | 754 | 18.873 | 42.963 | 13.027 | 1.00 | 39.76 | A | C |
| ATOM | 1007 | OD1 | ASP | A | 754 | 17.851 | 43.550 | 13.435 | 1.00 | 41.95 | A | O |
| ATOM | 1008 | OD2 | ASP | A | 754 | 19.762 | 42.534 | 13.794 | 1.00 | 38.96 | A | O |
| ATOM | 1009 | C | ASP | A | 754 | 20.325 | 41.811 | 9.646 | 1.00 | 33.47 | A | C |
| ATOM | 1010 | O | ASP | A | 754 | 20.234 | 40.617 | 9.350 | 1.00 | 29.70 | A | O |
| ATOM | 1011 | N | LEU | A | 755 | 20.341 | 42.779 | 8.734 | 1.00 | 32.73 | A | N |
| ATOM | 1012 | CA | LEU | A | 755 | 20.266 | 42.474 | 7.308 | 1.00 | 32.36 | A | C |
| ATOM | 1013 | CB | LEU | A | 755 | 20.171 | 43.765 | 6.490 | 1.00 | 32.35 | A | C |
| ATOM | 1014 | CG | LEU | A | 755 | 18.978 | 44.679 | 6.803 | 1.00 | 31.13 | A | C |
| ATOM | 1015 | CD1 | LEU | A | 755 | 18.974 | 45.851 | 5.832 | 1.00 | 29.37 | A | C |
| ATOM | 1016 | CD2 | LEU | A | 755 | 17.664 | 43.897 | 6.695 | 1.00 | 30.37 | A | C |
| ATOM | 1017 | C | LEU | A | 755 | 21.480 | 41.655 | 6.875 | 1.00 | 32.73 | A | C |
| ATOM | 1018 | O | LEU | A | 755 | 21.358 | 40.704 | 6.101 | 1.00 | 30.20 | A | O |
| ATOM | 1019 | N | LEU | A | 756 | 22.650 | 42.019 | 7.386 | 1.00 | 32.17 | A | N |
| ATOM | 1020 | CA | LEU | A | 756 | 23.870 | 41.297 | 7.062 | 1.00 | 32.95 | A | C |
| ATOM | 1021 | CB | LEU | A | 756 | 25.091 | 42.038 | 7.617 | 1.00 | 34.64 | A | C |
| ATOM | 1022 | CG | LEU | A | 756 | 25.654 | 43.190 | 6.777 | 1.00 | 38.54 | A | C |
| ATOM | 1023 | CD1 | LEU | A | 756 | 26.153 | 42.646 | 5.445 | 1.00 | 37.97 | A | C |
| ATOM | 1024 | CD2 | LEU | A | 756 | 24.597 | 44.255 | 6.546 | 1.00 | 41.90 | A | C |
| ATOM | 1025 | C | LEU | A | 756 | 23.822 | 39.873 | 7.612 | 1.00 | 32.82 | A | C |
| ATOM | 1026 | O | LEU | A | 756 | 24.357 | 38.950 | 6.998 | 1.00 | 30.75 | A | O |
| ATOM | 1027 | N | HIS | A | 757 | 23.188 | 39.699 | 8.770 | 1.00 | 33.50 | A | N |
| ATOM | 1028 | CA | HIS | A | 757 | 23.061 | 38.376 | 9.373 | 1.00 | 33.55 | A | C |
| ATOM | 1029 | CB | HIS | A | 757 | 22.512 | 38.474 | 10.801 | 1.00 | 38.05 | A | C |
| ATOM | 1030 | CG | HIS | A | 757 | 23.524 | 38.921 | 11.811 | 1.00 | 43.27 | A | C |
| ATOM | 1031 | CD2 | HIS | A | 757 | 23.406 | 39.716 | 12.901 | 1.00 | 45.11 | A | C |
| ATOM | 1032 | ND1 | HIS | A | 757 | 24.837 | 38.503 | 11.781 | 1.00 | 45.58 | A | N |
| ATOM | 1033 | CE1 | HIS | A | 757 | 25.485 | 39.021 | 12.811 | 1.00 | 46.89 | A | C |
| ATOM | 1034 | NE2 | HIS | A | 757 | 24.640 | 39.760 | 13.506 | 1.00 | 46.43 | A | N |
| ATOM | 1035 | C | HIS | A | 757 | 22.136 | 37.494 | 8.533 | 1.00 | 30.73 | A | C |
| ATOM | 1036 | O | HIS | A | 757 | 22.408 | 36.314 | 8.336 | 1.00 | 28.62 | A | O |
| ATOM | 1037 | N | PHE | A | 758 | 21.043 | 38.071 | 8.047 | 1.00 | 28.90 | A | N |
| ATOM | 1038 | CA | PHE | A | 758 | 20.101 | 37.335 | 7.213 | 1.00 | 28.09 | A | C |
| ATOM | 1039 | CE | PHE | A | 758 | 18.968 | 38.256 | 6.743 | 1.00 | 29.37 | A | C |
| ATOM | 1040 | CG | PHE | A | 758 | 17.996 | 38.640 | 7.822 | 1.00 | 28.43 | A | C |
| ATOM | 1041 | CD1 | PHE | A | 758 | 17.259 | 39.816 | 7.716 | 1.00 | 29.25 | A | C |
| ATOM | 1042 | CD2 | PHE | A | 758 | 17.781 | 37.812 | 8.917 | 1.00 | 30.71 | A | C |
| ATOM | 1043 | CE1 | PHE | A | 758 | 16.315 | 40.163 | 8.684 | 1.00 | 29.77 | A | C |
| ATOM | 1044 | CE2 | PHE | A | 758 | 16.837 | 38.149 | 9.893 | 1.00 | 31.39 | A | C |
| ATOM | 1045 | CZ | PHE | A | 758 | 16.104 | 39.327 | 9.775 | 1.00 | 29.70 | A | C |
| ATOM | 1046 | C | PHE | A | 758 | 20.865 | 36.839 | 5.995 | 1.00 | 26.37 | A | C |
| ATOM | 1047 | O | PHE | A | 758 | 20.763 | 35.674 | 5.607 | 1.00 | 25.55 | A | O |
| ATOM | 1048 | N | SER | A | 759 | 21.632 | 37.749 | 5.403 | 1.00 | 25.76 | A | N |
| ATOM | 1049 | CA | SER | A | 759 | 22.423 | 37.457 | 4.214 | 1.00 | 24.80 | A | C |
| ATOM | 1050 | CE | SER | A | 759 | 23.144 | 38.729 | 3.760 | 1.00 | 26.49 | A | C |
| ATOM | 1051 | OG | SER | A | 759 | 22.202 | 39.757 | 3.510 | 1.00 | 25.68 | A | O |
| ATOM | 1052 | C | SER | A | 759 | 23.422 | 36.322 | 4.435 | 1.00 | 24.92 | A | C |
| ATOM | 1053 | O | SER | A | 759 | 23.515 | 35.402 | 3.616 | 1.00 | 24.17 | A | O |
| ATOM | 1054 | N | SER | A | 760 | 24.162 | 36.386 | 5.537 | 1.00 | 24.42 | A | N |
| ATOM | 1055 | CA | SER | A | 760 | 25.152 | 35.355 | 5.867 | 1.00 | 25.72 | A | C |
| ATOM | 1056 | CE | SER | A | 760 | 25.992 | 35.779 | 7.083 | 1.00 | 29.13 | A | C |
| ATOM | 1057 | OG | SER | A | 760 | 26.884 | 36.825 | 6.744 | 1.00 | 37.41 | A | O |
| ATOM | 1058 | C | SER | A | 760 | 24.533 | 33.995 | 6.161 | 1.00 | 24.67 | A | C |
| ATOM | 1059 | O | SER | A | 760 | 25.049 | 32.967 | 5.728 | 1.00 | 25.74 | A | O |
| ATOM | 1060 | N | GLN | A | 761 | 23.439 | 33.980 | 6.913 | 1.00 | 25.18 | A | N |
| ATOM | 1061 | CA | GLN | A | 761 | 22.792 | 32.720 | 7.245 | 1.00 | 25.42 | A | C |
| ATOM | 1062 | CE | GLN | A | 761 | 21.656 | 32.970 | 8.242 | 1.00 | 26.19 | A | C |
| ATOM | 1063 | CG | GLN | A | 761 | 22.183 | 33.611 | 9.523 | 1.00 | 30.21 | A | C |
| ATOM | 1064 | CD | GLN | A | 761 | 21.100 | 34.078 | 10.461 | 1.00 | 29.64 | A | C |
| ATOM | 1065 | OE1 | GLN | A | 761 | 20.005 | 34.434 | 10.038 | 1.00 | 31.28 | A | O |
| ATOM | 1066 | NE2 | GLN | A | 761 | 21.413 | 34.108 | 11.754 | 1.00 | 36.46 | A | N |
| ATOM | 1067 | C | GLN | A | 761 | 22.297 | 31.993 | 5.995 | 1.00 | 24.08 | A | C |
| ATOM | 1068 | O | GLN | A | 761 | 22.461 | 30.782 | 5.881 | 1.00 | 23.84 | A | O |
| ATOM | 1069 | N | VAL | A | 762 | 21.719 | 32.725 | 5.046 | 1.00 | 23.44 | A | N |
| ATOM | 1070 | CA | VAL | A | 762 | 21.242 | 32.090 | 3.813 | 1.00 | 21.62 | A | C |
| ATOM | 1071 | CB | VAL | A | 762 | 20.371 | 33.069 | 2.975 | 1.00 | 20.64 | A | C |
| ATOM | 1072 | CG1 | VAL | A | 762 | 19.981 | 32.423 | 1.660 | 1.00 | 21.19 | A | C |
| ATOM | 1073 | CG2 | VAL | A | 762 | 19.136 | 33.459 | 3.760 | 1.00 | 20.60 | A | C |
| ATOM | 1074 | C | VAL | A | 762 | 22.417 | 31.600 | 2.959 | 1.00 | 21.49 | A | C |
| ATOM | 1075 | O | VAL | A | 762 | 22.353 | 30.536 | 2.335 | 1.00 | 21.71 | A | O |
| ATOM | 1076 | N | ALA | A | 763 | 23.496 | 32.373 | 2.930 | 1.00 | 21.14 | A | N |
| ATOM | 1077 | CA | ALA | A | 763 | 24.664 | 31.989 | 2.159 | 1.00 | 21.63 | A | C |
| ATOM | 1078 | CB | ALA | A | 763 | 25.691 | 33.130 | 2.144 | 1.00 | 22.00 | A | C |
| ATOM | 1079 | C | ALA | A | 763 | 25.275 | 30.725 | 2.769 | 1.00 | 21.20 | A | C |
| ATOM | 1080 | O | ALA | A | 763 | 25.807 | 29.878 | 2.058 | 1.00 | 22.45 | A | O |
| ATOM | 1081 | N | GLN | A | 764 | 25.182 | 30.605 | 4.089 | 1.00 | 22.80 | A | N |
| ATOM | 1082 | CA | GLN | A | 764 | 25.713 | 29.438 | 4.785 | 1.00 | 23.51 | A | C |
| ATOM | 1083 | CB | GLN | A | 764 | 25.666 | 29.662 | 6.298 | 1.00 | 27.64 | A | C |
| ATOM | 1084 | CG | GLN | A | 764 | 26.717 | 30.648 | 6.797 | 1.00 | 29.90 | A | C |
| ATOM | 1085 | CD | GLN | A | 764 | 26.526 | 31.029 | 8.253 | 1.00 | 35.51 | A | C |
| ATOM | 1086 | OE1 | GLN | A | 764 | 26.070 | 30.225 | 9.064 | 1.00 | 38.54 | A | O |
| ATOM | 1087 | NE2 | GLN | A | 764 | 26.891 | 32.257 | 8.595 | 1.00 | 37.48 | A | N |
| ATOM | 1088 | C | GLN | A | 764 | 24.880 | 28.225 | 4.398 | 1.00 | 24.02 | A | C |
| ATOM | 1089 | O | GLN | A | 764 | 25.420 | 27.153 | 4.106 | 1.00 | 23.48 | A | O |
| ATOM | 1090 | N | GLY | A | 765 | 23.563 | 28.401 | 4.380 | 1.00 | 23.88 | A | N |
| ATOM | 1091 | CA | GLY | A | 765 | 22.691 | 27.303 | 4.004 | 1.00 | 24.09 | A | C |
| ATOM | 1092 | C | GLY | A | 765 | 22.903 | 26.896 | 2.553 | 1.00 | 23.59 | A | C |
| ATOM | 1093 | O | GLY | A | 765 | 22.904 | 25.702 | 2.214 | 1.00 | 20.85 | A | O |
| ATOM | 1094 | N | MET | A | 766 | 23.077 | 27.890 | 1.687 | 1.00 | 23.50 | A | N |
| ATOM | 1095 | CA | MET | A | 766 | 23.301 | 27.623 | 0.268 | 1.00 | 23.20 | A | C |
| ATOM | 1096 | CB | MET | A | 766 | 23.186 | 28.918 | −0.547 | 1.00 | 23.05 | A | C |
| ATOM | 1097 | CG | MET | A | 766 | 21.766 | 29.486 | −0.596 | 1.00 | 21.86 | A | C |
| ATOM | 1098 | SD | MET | A | 766 | 20.548 | 28.295 | −1.235 | 1.00 | 24.09 | A | S |
| ATOM | 1099 | CE | MET | A | 766 | 21.039 | 28.204 | −2.963 | 1.00 | 22.29 | A | C |
| ATOM | 1100 | C | MET | A | 766 | 24.671 | 26.975 | 0.036 | 1.00 | 23.99 | A | C |
| ATOM | 1101 | O | MET | A | 766 | 24.821 | 26.125 | −0.836 | 1.00 | 21.94 | A | O |
| ATOM | 1102 | N | ALA | A | 767 | 25.669 | 27.379 | 0.812 | 1.00 | 24.25 | A | N |
| ATOM | 1103 | CA | ALA | A | 767 | 26.993 | 26.788 | 0.670 | 1.00 | 25.63 | A | C |
| ATOM | 1104 | CB | ALA | A | 767 | 28.001 | 27.508 | 1.565 | 1.00 | 29.76 | A | C |
| ATOM | 1105 | C | ALA | A | 767 | 26.876 | 25.315 | 1.067 | 1.00 | 26.90 | A | C |
| ATOM | 1106 | O | ALA | A | 767 | 27.538 | 24.459 | 0.499 | 1.00 | 26.08 | A | O |
| ATOM | 1107 | N | PHE | A | 768 | 26.012 | 25.029 | 2.038 | 1.00 | 27.54 | A | N |
| ATOM | 1108 | CA | PHE | A | 768 | 25.800 | 23.658 | 2.486 | 1.00 | 27.78 | A | C |
| ATOM | 1109 | CB | PHE | A | 768 | 24.877 | 23.636 | 3.706 | 1.00 | 29.16 | A | C |
| ATOM | 1110 | CG | PHE | A | 768 | 24.494 | 22.252 | 4.161 | 1.00 | 29.81 | A | C |
| ATOM | 1111 | CD1 | PHE | A | 768 | 25.430 | 21.411 | 4.757 | 1.00 | 32.92 | A | C |
| ATOM | 1112 | CD2 | PHE | A | 768 | 23.187 | 21.799 | 4.008 | 1.00 | 32.00 | A | C |
| ATOM | 1113 | CE1 | PHE | A | 768 | 25.067 | 20.137 | 5.197 | 1.00 | 33.70 | A | C |
| ATOM | 1114 | CE2 | PHE | A | 768 | 22.813 | 20.529 | 4.442 | 1.00 | 31.44 | A | C |
| ATOM | 1115 | CZ | PHE | A | 768 | 23.756 | 19.697 | 5.040 | 1.00 | 32.23 | A | C |
| ATOM | 1116 | C | PHE | A | 768 | 25.182 | 22.838 | 1.353 | 1.00 | 27.16 | A | C |
| ATOM | 1117 | O | PHE | A | 768 | 25.652 | 21.744 | 1.044 | 1.00 | 26.57 | A | O |
| ATOM | 1118 | N | LEU | A | 769 | 24.130 | 23.368 | 0.733 | 1.00 | 23.37 | A | N |
| ATOM | 1119 | CA | LEU | A | 769 | 23.483 | 22.654 | −0.362 | 1.00 | 25.00 | A | C |
| ATOM | 1120 | CB | LEU | A | 769 | 22.274 | 23.442 | −0.879 | 1.00 | 23.06 | A | C |
| ATOM | 1121 | CG | LEU | A | 769 | 21.060 | 23.467 | 0.065 | 1.00 | 22.02 | A | C |
| ATOM | 1122 | CD1 | LEU | A | 769 | 19.968 | 24.363 | −0.524 | 1.00 | 25.71 | A | C |
| ATOM | 1123 | CD2 | LEU | A | 769 | 20.529 | 22.056 | 0.255 | 1.00 | 25.33 | A | C |
| ATOM | 1124 | C | LEU | A | 769 | 24.483 | 22.427 | −1.492 | 1.00 | 23.96 | A | C |
| ATOM | 1125 | O | LEU | A | 769 | 24.595 | 21.322 | −2.029 | 1.00 | 25.23 | A | O |
| ATOM | 1126 | N | ALA | A | 770 | 25.212 | 23.478 | −1.845 | 1.00 | 25.25 | A | N |
| ATOM | 1127 | CA | ALA | A | 770 | 26.216 | 23.389 | −2.900 | 1.00 | 25.98 | A | C |
| ATOM | 1128 | CB | ALA | A | 770 | 26.959 | 24.703 | −3.017 | 1.00 | 27.54 | A | C |
| ATOM | 1129 | C | ALA | A | 770 | 27.209 | 22.258 | −2.620 | 1.00 | 27.89 | A | C |
| ATOM | 1130 | O | ALA | A | 770 | 27.600 | 21.526 | −3.536 | 1.00 | 28.96 | A | O |
| ATOM | 1131 | N | SER | A | 771 | 27.608 | 22.111 | −1.358 | 1.00 | 28.68 | A | N |
| ATOM | 1132 | CA | SER | A | 771 | 28.568 | 21.076 | −0.990 | 1.00 | 30.23 | A | C |
| ATOM | 1133 | CB | SER | A | 771 | 29.048 | 21.265 | 0.459 | 1.00 | 31.85 | A | C |
| ATOM | 1134 | OG | SER | A | 771 | 28.061 | 20.885 | 1.406 | 1.00 | 30.80 | A | O |
| ATOM | 1135 | C | SER | A | 771 | 28.007 | 19.670 | −1.164 | 1.00 | 32.60 | A | C |
| ATOM | 1136 | O | SER | A | 771 | 28.768 | 18.703 | −1.209 | 1.00 | 33.58 | A | O |
| ATOM | 1137 | N | LYS | A | 772 | 26.681 | 19.561 | −1.266 | 1.00 | 31.14 | A | N |
| ATOM | 1138 | CA | LYS | A | 772 | 26.012 | 18.272 | −1.435 | 1.00 | 31.62 | A | C |
| ATOM | 1139 | CB | LYS | A | 772 | 24.823 | 18.163 | −0.480 | 1.00 | 33.65 | A | C |
| ATOM | 1140 | CG | LYS | A | 772 | 25.125 | 18.456 | 0.970 | 1.00 | 35.63 | A | C |
| ATOM | 1141 | CD | LYS | A | 772 | 25.958 | 17.364 | 1.606 | 1.00 | 39.90 | A | C |
| ATOM | 1142 | CE | LYS | A | 772 | 26.145 | 17.627 | 3.094 | 1.00 | 40.74 | A | C |
| ATOM | 1143 | NZ | LYS | A | 772 | 26.888 | 16.514 | 3.746 | 1.00 | 43.28 | A | N |
| ATOM | 1144 | C | LYS | A | 772 | 25.486 | 18.142 | −2.860 | 1.00 | 31.71 | A | C |
| ATOM | 1145 | O | LYS | A | 772 | 24.688 | 17.250 | −3.158 | 1.00 | 32.73 | A | O |
| ATOM | 1146 | N | ASN | A | 773 | 25.930 | 19.039 | −3.734 | 1.00 | 31.26 | A | N |
| ATOM | 1147 | CA | ASN | A | 773 | 25.483 | 19.053 | −5.119 | 1.00 | 32.22 | A | C |
| ATOM | 1148 | CB | ASN | A | 773 | 25.993 | 17.816 | −5.860 | 1.00 | 35.15 | A | C |
| ATOM | 1149 | CG | ASN | A | 773 | 27.490 | 17.848 | −6.073 | 1.00 | 37.24 | A | C |
| ATOM | 1150 | OD1 | ASN | A | 773 | 28.046 | 18.874 | −6.462 | 1.00 | 40.31 | A | O |
| ATOM | 1151 | ND2 | ASN | A | 773 | 28.151 | 16.724 | −5.826 | 1.00 | 38.15 | A | N |
| ATOM | 1152 | C | ASN | A | 773 | 23.959 | 19.128 | −5.198 | 1.00 | 31.31 | A | C |
| ATOM | 1153 | O | ASN | A | 773 | 23.329 | 18.488 | −6.047 | 1.00 | 31.24 | A | O |
| ATOM | 1154 | N | CYS | A | 774 | 23.369 | 19.917 | −4.303 | 1.00 | 28.26 | A | N |
| ATOM | 1155 | CA | CYS | A | 774 | 21.917 | 20.078 | −4.277 | 1.00 | 25.72 | A | C |
| ATOM | 1156 | CB | CYS | A | 774 | 21.388 | 19.899 | −2.852 | 1.00 | 23.53 | A | C |
| ATOM | 1157 | SG | CYS | A | 774 | 19.588 | 19.885 | −2.687 | 1.00 | 27.47 | A | S |
| ATOM | 1158 | C | CYS | A | 774 | 21.537 | 21.461 | −4.789 | 1.00 | 26.86 | A | C |
| ATOM | 1159 | O | CYS | A | 774 | 22.038 | 22.471 | −4.285 | 1.00 | 25.39 | A | O |
| ATOM | 1160 | N | ILE | A | 775 | 20.672 | 21.504 | −5.800 | 1.00 | 26.10 | A | N |
| ATOM | 1161 | CA | ILE | A | 775 | 20.218 | 22.777 | −6.354 | 1.00 | 26.93 | A | C |
| ATOM | 1162 | CB | ILE | A | 775 | 20.118 | 22.730 | −7.896 | 1.00 | 27.55 | A | C |
| ATOM | 1163 | CG2 | ILE | A | 775 | 21.502 | 22.522 | −8.481 | 1.00 | 30.30 | A | C |
| ATOM | 1164 | CG1 | ILE | A | 775 | 19.183 | 21.608 | −8.343 | 1.00 | 30.30 | A | C |
| ATOM | 1165 | CD1 | ILE | A | 775 | 18.867 | 21.645 | −9.828 | 1.00 | 33.32 | A | C |
| ATOM | 1166 | C | ILE | A | 775 | 18.869 | 23.125 | −5.736 | 1.00 | 25.91 | A | C |
| ATOM | 1167 | O | ILE | A | 775 | 18.027 | 22.256 | −5.511 | 1.00 | 27.81 | A | O |
| ATOM | 1168 | N | HIS | A | 776 | 18.675 | 24.411 | −5.468 | 1.00 | 24.52 | A | N |
| ATOM | 1169 | CA | HIS | A | 776 | 17.477 | 24.909 | −4.815 | 1.00 | 23.29 | A | C |
| ATOM | 1170 | CB | HIS | A | 776 | 17.890 | 26.110 | −3.943 | 1.00 | 24.21 | A | C |
| ATOM | 1171 | CG | HIS | A | 776 | 16.791 | 26.669 | −3.097 | 1.00 | 24.03 | A | C |
| ATOM | 1172 | CD2 | HIS | A | 776 | 16.677 | 26.789 | −1.753 | 1.00 | 23.63 | A | C |
| ATOM | 1173 | ND1 | HIS | A | 776 | 15.655 | 27.239 | −3.631 | 1.00 | 23.23 | A | N |
| ATOM | 1174 | CE1 | HIS | A | 776 | 14.889 | 27.685 | −2.652 | 1.00 | 23.70 | A | C |
| ATOM | 1175 | NE2 | HIS | A | 776 | 15.485 | 27.426 | −1.503 | 1.00 | 22.77 | A | N |
| ATOM | 1176 | C | HIS | A | 776 | 16.362 | 25.288 | −5.798 | 1.00 | 23.79 | A | C |
| ATOM | 1177 | O | HIS | A | 776 | 15.219 | 24.855 | −5.649 | 1.00 | 22.64 | A | O |
| ATOM | 1178 | N | ARG | A | 777 | 16.716 | 26.102 | −6.785 | 1.00 | 24.29 | A | N |
| ATOM | 1179 | CA | ARG | A | 777 | 15.810 | 26.554 | −7.840 | 1.00 | 25.62 | A | C |
| ATOM | 1180 | CB | ARG | A | 777 | 15.223 | 25.333 | −8.574 | 1.00 | 27.57 | A | C |
| ATOM | 1181 | CG | ARG | A | 777 | 16.303 | 24.507 | −9.281 | 1.00 | 32.08 | A | C |
| ATOM | 1182 | CD | ARG | A | 777 | 15.733 | 23.457 | −10.236 | 1.00 | 34.83 | A | C |
| ATOM | 1183 | NE | ARG | A | 777 | 15.019 | 22.381 | −9.551 | 1.00 | 38.69 | A | N |
| ATOM | 1184 | CZ | ARG | A | 777 | 14.469 | 21.342 | −10.177 | 1.00 | 41.11 | A | C |
| ATOM | 1185 | NH1 | ARG | A | 777 | 14.555 | 21.244 | −11.499 | 1.00 | 40.81 | A | N |
| ATOM | 1186 | NH2 | ARG | A | 777 | 13.834 | 20.402 | −9.486 | 1.00 | 39.14 | A | N |
| ATOM | 1187 | C | ARG | A | 777 | 14.699 | 27.559 | −7.518 | 1.00 | 25.50 | A | C |
| ATOM | 1188 | O | ARG | A | 777 | 13.957 | 27.949 | −8.417 | 1.00 | 26.60 | A | O |
| ATOM | 1189 | N | ASP | A | 778 | 14.561 | 27.989 | −6.261 | 1.00 | 21.34 | A | N |
| ATOM | 1190 | CA | ASP | A | 778 | 13.541 | 28.994 | −5.946 | 1.00 | 20.57 | A | C |
| ATOM | 1191 | CB | ASP | A | 778 | 12.209 | 28.335 | −5.540 | 1.00 | 20.83 | A | C |
| ATOM | 1192 | CG | ASP | A | 778 | 11.001 | 29.288 | −5.677 | 1.00 | 22.46 | A | C |
| ATOM | 1193 | OD1 | ASP | A | 778 | 9.858 | 28.863 | −5.387 | 1.00 | 23.53 | A | O |
| ATOM | 1194 | OD2 | ASP | A | 778 | 11.189 | 30.462 | −6.065 | 1.00 | 23.50 | A | O |
| ATOM | 1195 | C | ASP | A | 778 | 14.054 | 29.904 | −4.811 | 1.00 | 19.92 | A | C |
| ATOM | 1196 | O | ASP | A | 778 | 13.337 | 30.200 | −3.868 | 1.00 | 20.94 | A | O |
| ATOM | 1197 | N | VAL | A | 779 | 15.309 | 30.322 | −4.907 | 1.00 | 19.46 | A | N |
| ATOM | 1198 | CA | VAL | A | 779 | 15.887 | 31.200 | −3.882 | 1.00 | 20.04 | A | C |
| ATOM | 1199 | CB | VAL | A | 779 | 17.407 | 31.351 | −4.073 | 1.00 | 18.09 | A | C |
| ATOM | 1200 | CG1 | VAL | A | 779 | 17.970 | 32.391 | −3.084 | 1.00 | 17.98 | A | C |
| ATOM | 1201 | CG2 | VAL | A | 779 | 18.078 | 29.991 | −3.832 | 1.00 | 21.88 | A | C |
| ATOM | 1202 | C | VAL | A | 779 | 15.196 | 32.556 | −4.017 | 1.00 | 20.85 | A | C |
| ATOM | 1203 | O | VAL | A | 779 | 15.218 | 33.170 | −5.085 | 1.00 | 21.27 | A | O |
| ATOM | 1204 | N | ALA | A | 780 | 14.551 | 32.984 | −2.933 | 1.00 | 18.99 | A | N |
| ATOM | 1205 | CA | ALA | A | 780 | 13.814 | 34.242 | −2.879 | 1.00 | 19.61 | A | C |
| ATOM | 1206 | CB | ALA | A | 780 | 12.538 | 34.141 | −3.710 | 1.00 | 17.53 | A | C |
| ATOM | 1207 | CAL | A | A | 780 | 13.463 | 34.505 | −1.413 | 1.00 | 19.77 | A | C |
| ATOM | 1208 | O | ALA | A | 780 | 13.398 | 33.571 | −0.612 | 1.00 | 19.86 | A | O |
| ATOM | 1209 | N | ALA | A | 781 | 13.237 | 35.766 | −1.065 | 1.00 | 20.41 | A | N |
| ATOM | 1210 | CA | ALA | A | 781 | 12.911 | 36.121 | 0.315 | 1.00 | 20.37 | A | C |
| ATOM | 1211 | CB | ALA | A | 781 | 12.757 | 37.652 | 0.448 | 1.00 | 19.44 | A | C |
| ATOM | 1212 | C | ALA | A | 781 | 11.647 | 35.408 | 0.811 | 1.00 | 20.67 | A | C |
| ATOM | 1213 | O | ALA | A | 781 | 11.548 | 35.061 | 1.993 | 1.00 | 21.97 | A | O |
| ATOM | 1214 | N | ARG | A | 782 | 10.690 | 35.174 | −0.082 | 1.00 | 21.06 | A | N |
| ATOM | 1215 | CA | ARG | A | 782 | 9.462 | 34.485 | 0.313 | 1.00 | 20.10 | A | C |
| ATOM | 1216 | CB | ARG | A | 782 | 8.451 | 34.471 | −0.840 | 1.00 | 20.60 | A | C |
| ATOM | 1217 | CG | ARG | A | 782 | 8.966 | 33.736 | 2.075 | 1.00 | 21.22 | A | C |
| ATOM | 1218 | CD | ARG | A | 782 | 7.942 | 33.633 | −3.195 | 1.00 | 20.84 | A | C |
| ATOM | 1219 | NE | ARG | A | 782 | 8.589 | 33.082 | −4.380 | 1.00 | 21.81 | A | N |
| ATOM | 1220 | CZ | ARG | A | 782 | 9.295 | 33.797 | −5.250 | 1.00 | 22.06 | A | C |
| ATOM | 1221 | NH1 | ARG | A | 782 | 9.437 | 35.106 | −5.091 | 1.00 | 21.34 | A | N |
| ATOM | 1222 | NH2 | ARG | A | 782 | 9.897 | 33.196 | −6.261 | 1.00 | 20.10 | A | N |
| ATOM | 1223 | C | ARG | A | 782 | 9.799 | 33.044 | 0.715 | 1.00 | 21.70 | A | C |
| ATOM | 1224 | O | ARG | A | 782 | 9.009 | 32.384 | 1.384 | 1.00 | 22.67 | A | O |
| ATOM | 1225 | N | ASN | A | 783 | 10.982 | 32.573 | 0.322 | 1.00 | 21.65 | A | N |
| ATOM | 1226 | CA | ASN | A | 783 | 11.410 | 31.205 | 0.638 | 1.00 | 21.44 | A | C |
| ATOM | 1227 | CB | ASN | A | 783 | 11.822 | 30.481 | −0.647 | 1.00 | 22.37 | A | C |
| ATOM | 1228 | CG | ASN | A | 783 | 10.619 | 30.092 | −1.498 | 1.00 | 23.95 | A | C |
| ATOM | 1229 | OD1 | ASN | A | 783 | 9.558 | 29.736 | −0.961 | 1.00 | 21.09 | A | O |
| ATOM | 1230 | ND2 | ASN | A | 783 | 10.776 | 30.143 | −2.832 | 1.00 | 21.48 | A | N |
| ATOM | 1231 | C | ASN | A | 783 | 12.498 | 31.082 | 1.706 | 1.00 | 21.95 | A | C |
| ATOM | 1232 | O | ASN | A | 783 | 13.195 | 30.065 | 1.812 | 1.00 | 23.59 | A | O |
| ATOM | 1233 | N | VAL | A | 784 | 12.644 | 32.134 | 2.499 | 1.00 | 21.62 | A | N |
| ATOM | 1234 | CA | VAL | A | 784 | 13.594 | 32.132 | 3.597 | 1.00 | 20.43 | A | C |
| ATOM | 1235 | CB | VAL | A | 784 | 14.584 | 33.314 | 3.521 | 1.00 | 21.59 | A | C |
| ATOM | 1236 | CG1 | VAL | A | 784 | 15.494 | 33.301 | 4.749 | 1.00 | 20.70 | A | C |
| ATOM | 1237 | CG2 | VAL | A | 784 | 15.440 | 33.210 | 2.241 | 1.00 | 20.73 | A | C |
| ATOM | 1238 | C | VAL | A | 784 | 12.698 | 32.288 | 4.827 | 1.00 | 22.32 | A | C |
| ATOM | 1239 | O | VAL | A | 784 | 11.898 | 33.224 | 4.907 | 1.00 | 21.28 | A | O |
| ATOM | 1240 | N | LEU | A | 785 | 12.807 | 31.354 | 5.765 | 1.00 | 22.34 | A | N |
| ATOM | 1241 | CA | LEU | A | 785 | 11.980 | 31.409 | 6.965 | 1.00 | 25.69 | A | C |
| ATOM | 1242 | CB | LEU | A | 785 | 11.335 | 30.042 | 7.234 | 1.00 | 25.59 | A | C |
| ATOM | 1243 | CG | LEU | A | 785 | 9.974 | 29.741 | 6.589 | 1.00 | 31.99 | A | C |
| ATOM | 1244 | CD1 | LEU | A | 785 | 9.820 | 30.454 | 5.265 | 1.00 | 27.32 | A | C |
| ATOM | 1245 | CD2 | LEU | A | 785 | 9.803 | 28.232 | 6.438 | 1.00 | 26.69 | A | C |
| ATOM | 1246 | C | LEU | A | 785 | 12.815 | 31.838 | 8.145 | 1.00 | 25.12 | A | C |
| ATOM | 1247 | O | LEU | A | 785 | 14.037 | 31.683 | 8.145 | 1.00 | 25.05 | A | O |
| ATOM | 1248 | N | LEU | A | 786 | 12.148 | 32.385 | 9.151 | 1.00 | 25.64 | A | N |
| ATOM | 1249 | CA | LEU | A | 786 | 12.839 | 32.858 | 1.0.334 | 1.00 | 26.80 | A | C |
| ATOM | 1250 | CB | LEU | A | 786 | 12.629 | 34.367 | 10.500 | 1.00 | 27.64 | A | C |
| ATOM | 1251 | CG | LEU | A | 786 | 13.149 | 35.275 | 9.381 | 1.00 | 29.65 | A | C |
| ATOM | 1252 | CD1 | LEU | A | 786 | 12.801 | 36.727 | 9.711 | 1.00 | 29.22 | A | C |
| ATOM | 1253 | CD2 | LEU | A | 786 | 14.651 | 35.106 | 9.228 | 1.00 | 28.29 | A | C |
| ATOM | 1254 | C | LEU | A | 786 | 12.305 | 32.113 | 11.540 | 1.00 | 25.15 | A | C |
| ATOM | 1255 | O | LEU | A | 786 | 11.094 | 32.045 | 11.757 | 1.00 | 27.13 | A | O |
| ATOM | 1256 | N | THR | A | 787 | 13.212 | 31.535 | 12.311 | 1.00 | 28.10 | A | N |
| ATOM | 1257 | CA | THR | A | 787 | 12.818 | 30.777 | 13.487 | 1.00 | 30.32 | A | C |
| ATOM | 1258 | GB | THR | A | 787 | 13.409 | 29.340 | 13.424 | 1.00 | 30.73 | A | C |
| ATOM | 1259 | OG1 | THR | A | 787 | 12.862 | 28.546 | 14.480 | 1.00 | 32.26 | A | O |
| ATOM | 1260 | CG2 | THR | A | 787 | 14.926 | 29.372 | 13.548 | 1.00 | 29.22 | A | C |
| ATOM | 1261 | C | THR | A | 787 | 13.305 | 31.507 | 14.740 | 1.00 | 32.88 | A | C |
| ATOM | 1262 | O | THR | A | 787 | 13.603 | 32.704 | 14.689 | 1.00 | 31.75 | A | O |
| ATOM | 1263 | N | ASN | A | 788 | 13.376 | 30.790 | 15.858 | 1.00 | 35.64 | A | N |
| ATOM | 1264 | CA | ASN | A | 788 | 13.836 | 31.360 | 17.123 | 1.00 | 38.20 | A | C |
| ATOM | 1265 | CB | ASN | A | 788 | 14.132 | 30.236 | 18.118 | 1.00 | 39.74 | A | C |
| ATOM | 1266 | CG | ASN | A | 788 | 13.104 | 29.128 | 18.061 | 1.00 | 42.58 | A | C |
| ATOM | 1267 | OD1 | ASN | A | 788 | 11.937 | 29.327 | 18.405 | 1.00 | 45.37 | A | O |
| ATOM | 1268 | ND2 | ASN | A | 788 | 13.530 | 27.951 | 17.616 | 1.00 | 44.02 | A | N |
| ATOM | 1269 | C | ASN | A | 788 | 15.102 | 32.187 | 16.906 | 1.00 | 38.65 | A | C |
| ATOM | 1270 | O | ASN | A | 788 | 16.014 | 31.763 | 16.188 | 1.00 | 39.21 | A | O |
| ATOM | 1271 | N | GLY | A | 789 | 15.156 | 33.362 | 17.527 | 1.00 | 39.11 | A | N |
| ATOM | 1272 | CA | GLY | A | 789 | 16.316 | 34.225 | 17.385 | 1.00 | 38.28 | A | C |
| ATOM | 1273 | C | GLY | A | 789 | 16.392 | 34.900 | 16.025 | 1.00 | 37.99 | A | C |
| ATOM | 1274 | O | GLY | A | 789 | 17.414 | 35.498 | 15.670 | 1.00 | 37.34 | A | O |
| ATOM | 1275 | N | HIS | A | 790 | 15.308 | 34.807 | 15.262 | 1.00 | 37.61 | A | N |
| ATOM | 1276 | CA | HIS | A | 790 | 15.256 | 35.404 | 13.933 | 1.00 | 38.10 | A | C |
| ATOM | 1277 | GB | HIS | A | 790 | 15.373 | 36.924 | 14.048 | 1.00 | 39.56 | A | C |
| ATOM | 1278 | CG | HIS | A | 790 | 14.249 | 37.546 | 14.813 | 1.00 | 41.47 | A | C |
| ATOM | 1279 | CD2 | HIS | A | 790 | 14.153 | 37.915 | 16.113 | 1.00 | 40.90 | A | C |
| ATOM | 1280 | ND1 | HIS | A | 790 | 13.009 | 37.774 | 14.257 | 1.00 | 42.62 | A | N |
| ATOM | 1281 | CE1 | HIS | A | 790 | 12.195 | 38.254 | 15.181 | 1.00 | 43.46 | A | C |
| ATOM | 1282 | NE2 | HIS | A | 790 | 12.865 | 38.348 | 16.316 | 1.00 | 43.98 | A | N |
| ATOM | 1283 | C | HIS | A | 790 | 16.360 | 34.851 | 13.034 | 1.00 | 36.17 | A | C |
| ATOM | 1284 | O | HIS | A | 790 | 16.878 | 35.548 | 12.162 | 1.00 | 37.76 | A | O |
| ATOM | 1285 | N | VAL | A | 791 | 16.724 | 33.595 | 13.260 | 1.00 | 33.73 | A | N |
| ATOM | 1286 | CA | VAL | A | 791 | 17.749 | 32.942 | 12.459 | 1.00 | 30.86 | A | C |
| ATOM | 1287 | GB | VAL | A | 791 | 18.351 | 31.729 | 13.205 | 1.00 | 31.65 | A | C |
| ATOM | 1288 | CG1 | VAL | A | 791 | 19.373 | 31.026 | 12.330 | 1.00 | 31.86 | A | C |
| ATOM | 1289 | CG2 | VAL | A | 791 | 19.010 | 32.195 | 14.500 | 1.00 | 33.10 | A | C |
| ATOM | 1290 | C | VAL | A | 791 | 17.084 | 32.474 | 11.165 | 1.00 | 27.95 | A | C |
| ATOM | 1291 | O | VAL | A | 791 | 15.979 | 31.926 | 11.188 | 1.00 | 25.29 | A | O |
| ATOM | 1292 | N | ALA | A | 792 | 17.753 | 32.702 | 10.040 | 1.00 | 26.39 | A | N |
| ATOM | 1293 | CA | ALA | A | 792 | 17.206 | 32.314 | 8.744 | 1.00 | 26.00 | A | C |
| ATOM | 1294 | GB | ALA | A | 792 | 17.776 | 33.211 | 7.653 | 1.00 | 24.89 | A | C |
| ATOM | 1295 | C | ALA | A | 792 | 17.456 | 30.856 | 8.389 | 1.00 | 23.83 | A | C |
| ATOM | 1296 | O | ALA | A | 792 | 18.504 | 30.294 | 8.701 | 1.00 | 26.36 | A | O |
| ATOM | 1297 | N | LYS | A | 793 | 16.480 | 30.246 | 7.730 | 1.00 | 26.04 | A | N |
| ATOM | 1298 | CA | LYS | A | 793 | 16.594 | 28.859 | 7.294 | 1.00 | 25.20 | A | C |
| ATOM | 1299 | GB | LYS | A | 793 | 15.860 | 27.915 | 8.254 | 1.00 | 27.35 | A | C |
| ATOM | 1300 | CG | LYS | A | 793 | 16.578 | 27.637 | 9.580 | 1.00 | 28.50 | A | C |
| ATOM | 1301 | CD | LYS | A | 793 | 15.648 | 26.895 | 10.527 | 1.00 | 27.71 | A | C |
| ATOM | 1302 | GE | LYS | A | 793 | 16.360 | 26.431 | 11.789 | 1.00 | 29.22 | A | C |
| ATOM | 1303 | NZ | LYS | A | 793 | 17.314 | 25.326 | 11.505 | 1.00 | 26.71 | A | N |
| ATOM | 1304 | C | LYS | A | 793 | 15.962 | 28.745 | 5.909 | 1.00 | 25.45 | A | C |
| ATOM | 1305 | O | LYS | A | 793 | 15.032 | 29.481 | 5.584 | 1.00 | 24.20 | A | O |
| ATOM | 1306 | N | ILE | A | 794 | 16.482 | 27.832 | 5.095 | 1.00 | 24.93 | A | N |
| ATOM | 1307 | GA | ILE | A | 794 | 15.937 | 27.617 | 3.762 | 1.00 | 25.00 | A | C |
| ATOM | 1308 | GB | ILE | A | 794 | 17.001 | 27.864 | 2.664 | 1.00 | 23.98 | A | C |
| ATOM | 1309 | CG2 | ILE | A | 794 | 17.407 | 29.337 | 2.660 | 1.00 | 27.78 | A | C |
| ATOM | 1310 | CG1 | ILE | A | 794 | 18.227 | 26.976 | 2.901 | 1.00 | 26.09 | A | C |
| ATOM | 1311 | CD1 | ILE | A | 794 | 19.333 | 27.143 | 1.871 | 1.00 | 26.75 | A | C |
| ATOM | 1312 | C | ILE | A | 794 | 15.431 | 26.175 | 3.692 | 1.00 | 23.46 | A | C |
| ATOM | 1313 | O | ILE | A | 794 | 15.861 | 25.323 | 4.460 | 1.00 | 22.24 | A | O |
| ATOM | 1314 | N | GLY | A | 795 | 14.502 | 25.914 | 2.786 | 1.00 | 23.49 | A | N |
| ATOM | 1315 | CA | GLY | A | 795 | 13.971 | 24.567 | 2.656 | 1.00 | 23.61 | A | C |
| ATOM | 1316 | C | GLY | A | 795 | 13.200 | 24.430 | 1.364 | 1.00 | 24.05 | A | C |
| ATOM | 1317 | O | GLY | A | 795 | 13.056 | 25.402 | 0.626 | 1.00 | 22.55 | A | O |
| ATOM | 1318 | N | ASP | A | 796 | 12.727 | 23.226 | 1.064 | 1.00 | 24.93 | A | N |
| ATOM | 1319 | CA | ASP | A | 796 | 11.936 | 23.031 | −0.143 | 1.00 | 25.91 | A | C |
| ATOM | 1320 | CB | ASP | A | 796 | 12.102 | 21.633 | 0.712 | 1.00 | 26.18 | A | C |
| ATOM | 1321 | CG | ASP | A | 796 | 11.526 | 21.523 | −2.108 | 1.00 | 29.94 | A | C |
| ATOM | 1322 | OD1 | ASP | A | 796 | 10.592 | 22.300 | −2.424 | 1.00 | 31.04 | A | O |
| ATOM | 1323 | OD2 | ASP | A | 796 | 11.997 | 20.674 | −2.882 | 1.00 | 30.49 | A | O |
| ATOM | 1324 | C | ASP | A | 796 | 10.485 | 23.220 | 0.271 | 1.00 | 25.51 | A | C |
| ATOM | 1325 | O | ASP | A | 796 | 9.907 | 22.356 | 0.925 | 1.00 | 27.31 | A | O |
| ATOM | 1326 | N | PHE | A | 797 | 9.901 | 24.347 | −0.116 | 1.00 | 25.97 | A | N |
| ATOM | 1327 | CA | PHE | A | 797 | 8.525 | 24.665 | 0.248 | 1.00 | 28.13 | A | C |
| ATOM | 1328 | CB | PHE | A | 797 | 8.462 | 26.094 | 0.807 | 1.00 | 26.89 | A | C |
| ATOM | 1329 | CG | PHE | A | 797 | 9.492 | 26.383 | 1.863 | 1.00 | 26.54 | A | C |
| ATOM | 1330 | CD1 | PHE | A | 797 | 10.119 | 27.629 | 1.913 | 1.00 | 26.78 | A | C |
| ATOM | 1331 | CD2 | PHE | A | 797 | 9.846 | 25.418 | 2.805 | 1.00 | 26.85 | A | C |
| ATOM | 1332 | CE1 | PHE | A | 797 | 11.080 | 27.905 | 2.882 | 1.00 | 27.17 | A | C |
| ATOM | 1333 | CE2 | PHE | A | 797 | 10.807 | 25.686 | 3.780 | 1.00 | 24.82 | A | C |
| ATOM | 1334 | CZ | PHE | A | 797 | 11.427 | 26.933 | 3.818 | 1.00 | 26.11 | A | C |
| ATOM | 1335 | C | PHE | A | 797 | 7.588 | 24.554 | −0.943 | 1.00 | 29.04 | A | C |
| ATOM | 1336 | O | PHE | A | 797 | 6.413 | 24.898 | −0.847 | 1.00 | 30.66 | A | O |
| ATOM | 1337 | N | GLY | A | 798 | 8.112 | 24.068 | −2.062 | 1.00 | 30.54 | A | N |
| ATOM | 1338 | CA | GLY | A | 798 | 7.318 | 23.941 | −3.271 | 1.00 | 32.47 | A | C |
| ATOM | 1339 | C | GLY | A | 798 | 5.956 | 23.265 | −3.183 | 1.00 | 33.84 | A | C |
| ATOM | 1340 | O | GLY | A | 798 | 4.998 | 23.730 | −3.794 | 1.00 | 32.72 | A | O |
| ATOM | 1341 | N | LEU | A | 799 | 5.850 | 22.174 | −2.434 | 1.00 | 34.76 | A | N |
| ATOM | 1342 | CA | LEU | A | 799 | 4.572 | 21.472 | −2.341 | 1.00 | 36.51 | A | C |
| ATOM | 1343 | CB | LEU | A | 799 | 4.781 | 20.083 | −1.728 | 1.00 | 40.67 | A | C |
| ATOM | 1344 | CG | LEU | A | 799 | 5.530 | 19.112 | −2.654 | 1.00 | 43.92 | A | C |
| ATOM | 1345 | CD1 | LEU | A | 799 | 5.895 | 17.839 | −1.909 | 1.00 | 46.10 | A | C |
| ATOM | 1346 | CD2 | LEU | A | 799 | 4.664 | 18.793 | −3.865 | 1.00 | 47.14 | A | C |
| ATOM | 1347 | C | LEU | A | 799 | 3.482 | 22.232 | −1.585 | 1.00 | 36.55 | A | C |
| ATOM | 1348 | O | LEU | A | 799 | 2.297 | 21.916 | −1.717 | 1.00 | 36.02 | A | O |
| ATOM | 1349 | N | ALA | A | 800 | 3.877 | 23.241 | −0.812 | 1.00 | 33.43 | A | N |
| ATOM | 1350 | CA | ALA | A | 800 | 2.926 | 24.039 | −0.053 | 1.00 | 33.51 | A | C |
| ATOM | 1351 | CB | ALA | A | 800 | 3.294 | 24.022 | 1.420 | 1.00 | 33.66 | A | C |
| ATOM | 1352 | C | ALA | A | 800 | 2.864 | 25.480 | −0.551 | 1.00 | 31.46 | A | C |
| ATOM | 1353 | O | ALA | A | 800 | 2.196 | 26.320 | 0.047 | 1.00 | 32.62 | A | O |
| ATOM | 1354 | N | ARG | A | 801 | 3.571 | 25.757 | −1.642 | 1.00 | 31.79 | A | N |
| ATOM | 1355 | CA | ARG | A | 801 | 3.618 | 27.091 | −2.230 | 1.00 | 31.14 | A | C |
| ATOM | 1356 | CB | ARG | A | 801 | 4.909 | 27.239 | −3.047 | 1.00 | 31.63 | A | C |
| ATOM | 1357 | CG | ARG | A | 801 | 5.042 | 28.528 | −3.851 | 1.00 | 35.16 | A | C |
| ATOM | 1358 | CD | ARG | A | 801 | 5.013 | 29.762 | −2.966 | 1.00 | 33.53 | A | C |
| ATOM | 1359 | NE | ARG | A | 801 | 6.116 | 29.799 | −2.007 | 1.00 | 34.21 | A | N |
| ATOM | 1360 | CZ | ARG | A | 801 | 6.214 | 30.694 | −1.028 | 1.00 | 32.78 | A | C |
| ATOM | 1361 | NH1 | ARG | A | 801 | 5.280 | 31.621 | −0.884 | 1.00 | 34.78 | A | N |
| ATOM | 1362 | NH2 | ARG | A | 801 | 7.236 | 30.660 | −0.186 | 1.00 | 31.78 | A | N |
| ATOM | 1363 | C | ARG | A | 801 | 2.395 | 27.316 | −3.119 | 1.00 | 31.19 | A | C |
| ATOM | 1364 | O | ARG | A | 801 | 2.117 | 26.520 | −4.015 | 1.00 | 29.81 | A | O |
| ATOM | 1365 | N | ASP | A | 802 | 1.665 | 28.398 | −2.860 | 1.00 | 31.50 | A | N |
| ATOM | 1366 | CA | ASP | A | 802 | 0.471 | 28.730 | −3.631 | 1.00 | 32.83 | A | C |
| ATOM | 1367 | CB | ASP | A | 802 | −0.420 | 29.668 | −2.806 | 1.00 | 34.72 | A | C |
| ATOM | 1368 | CG | ASP | A | 802 | −1.677 | 30.092 | −3.539 | 1.00 | 35.12 | A | C |
| ATOM | 1369 | OD1 | ASP | A | 802 | −2.226 | 29.260 | −4.312 | 1.00 | 34.82 | A | O |
| ATOM | 1370 | OD2 | ASP | A | 802 | −2.132 | 31.240 | −3.321 | 1.00 | 34.71 | A | O |
| ATOM | 1371 | C | ASP | A | 802 | 0.912 | 29.392 | −4.933 | 1.00 | 33.93 | A | C |
| ATOM | 1372 | O | ASP | A | 802 | 0.741 | 30.593 | −5.127 | 1.00 | 35.98 | A | O |
| ATOM | 1373 | N | ILE | A | 803 | 1.468 | 28.578 | −5.824 | 1.00 | 31.80 | A | N |
| ATOM | 1374 | CA | ILE | A | 803 | 1.992 | 29.020 | −7.114 | 1.00 | 32.36 | A | C |
| ATOM | 1375 | CB | ILE | A | 803 | 2.828 | 27.882 | −7.752 | 1.00 | 35.59 | A | C |
| ATOM | 1376 | CG2 | ILE | A | 803 | 3.310 | 28.286 | −9.123 | 1.00 | 33.31 | A | C |
| ATOM | 1377 | CG1 | ILE | A | 803 | 4.021 | 27.552 | −6.853 | 1.00 | 37.06 | A | C |
| ATOM | 1378 | CD1 | ILE | A | 803 | 4.836 | 26.374 | −7.324 | 1.00 | 41.55 | A | C |
| ATOM | 1379 | C | ILE | A | 803 | 0.965 | 29.501 | −8.145 | 1.00 | 30.44 | A | C |
| ATOM | 1380 | O | ILE | A | 803 | 1.249 | 30.394 | −8.942 | 1.00 | 31.65 | A | O |
| ATOM | 1381 | N | MET | A | 804 | −0.215 | 28.899 | −8.134 | 1.00 | 27.98 | A | N |
| ATOM | 1382 | CA | MET | A | 804 | −1.262 | 29.252 | −9.083 | 1.00 | 28.49 | A | C |
| ATOM | 1383 | CB | MET | A | 804 | −2.415 | 28.246 | −8.982 | 1.00 | 29.43 | A | C |
| ATOM | 1384 | CG | MET | A | 804 | −2.031 | 26.813 | −9.349 | 1.00 | 31.07 | A | C |
| ATOM | 1385 | SD | MET | A | 804 | −1.429 | 26.680 | −11.051 | 1.00 | 35.46 | A | S |
| ATOM | 1386 | CE | MET | A | 804 | −2.994 | 26.637 | −11.934 | 1.00 | 37.06 | A | C |
| ATOM | 1387 | C | MET | A | 804 | −1.811 | 30.659 | −8.889 | 1.00 | 27.47 | A | C |
| ATOM | 1388 | O | MET | A | 804 | −2.190 | 31.322 | −9.856 | 1.00 | 28.22 | A | O |
| ATOM | 1389 | N | ASN | A | 805 | −1.844 | 31.109 | −7.639 | 1.00 | 26.10 | A | N |
| ATOM | 1390 | CA | ASN | A | 805 | −2.389 | 32.412 | −7.311 | 1.00 | 27.17 | A | C |
| ATOM | 1391 | CB | ASN | A | 805 | −3.426 | 32.245 | −6.195 | 1.00 | 27.53 | A | C |
| ATOM | 1392 | CG | ASN | A | 805 | −4.558 | 31.307 | −6.600 | 1.00 | 25.36 | A | C |
| ATOM | 1393 | OD1 | ASN | A | 805 | −5.229 | 31.536 | −7.604 | 1.00 | 25.30 | A | O |
| ATOM | 1394 | ND2 | ASN | A | 805 | −4.766 | 30.250 | −5.831 | 1.00 | 25.09 | A | N |
| ATOM | 1395 | C | ASN | A | 805 | −1.357 | 33.476 | −6.954 | 1.00 | 29.06 | A | C |
| ATOM | 1396 | O | ASN | A | 805 | −1.714 | 34.576 | −6.534 | 1.00 | 29.22 | A | O |
| ATOM | 1397 | N | ASP | A | 806 | −0.079 | 33.149 | −7.136 | 1.00 | 29.30 | A | N |
| ATOM | 1398 | CA | ASP | A | 806 | 0.998 | 34.096 | −6.866 | 1.00 | 28.84 | A | C |
| ATOM | 1399 | CB | ASP | A | 806 | 2.157 | 33.412 | −6.133 | 1.00 | 30.98 | A | C |
| ATOM | 1400 | CG | ASP | A | 806 | 3.219 | 34.403 | −5.679 | 1.00 | 29.20 | A | C |
| ATOM | 1401 | OD1 | ASP | A | 806 | 3.387 | 35.438 | −6.355 | 1.00 | 29.11 | A | O |
| ATOM | 1402 | OD2 | ASP | A | 806 | 3.883 | 34.142 | −4.658 | 1.00 | 30.93 | A | O |
| ATOM | 1403 | C | ASP | A | 806 | 1.481 | 34.603 | −8.222 | 1.00 | 29.89 | A | C |
| ATOM | 1404 | O | ASP | A | 806 | 2.066 | 33.848 | −8.992 | 1.00 | 28.06 | A | O |
| ATOM | 1405 | N | SER | A | 807 | 1.237 | 35.881 | −8.507 | 1.00 | 29.68 | A | N |
| ATOM | 1406 | CA | SER | A | 807 | 1.620 | 36.473 | −9.787 | 1.00 | 31.34 | A | C |
| ATOM | 1407 | CB | SER | A | 807 | 1.022 | 37.880 | −9.917 | 1.00 | 32.84 | A | C |
| ATOM | 1408 | OG | SER | A | 807 | 1.554 | 38.752 | −8.939 | 1.00 | 36.12 | A | O |
| ATOM | 1409 | C | SER | A | 807 | 3.1253 | 6.521 | −10.043 | 1.00 | 31.42 | A | C |
| ATOM | 1410 | O | SER | A | 807 | 3.556 | 36.816 | −11.158 | 1.00 | 31.90 | A | O |
| ATOM | 1411 | N | ASN | A | 808 | 3.921 | 36.232 | −9.016 | 1.00 | 30.84 | A | N |
| ATOM | 1412 | CA | ASN | A | 808 | 5.373 | 36.209 | −9.163 | 1.00 | 30.18 | A | C |
| ATOM | 1413 | CB | ASN | A | 808 | 6.054 | 36.257 | −7.795 | 1.00 | 32.39 | A | C |
| ATOM | 1414 | CG | ASN | A | 808 | 6.000 | 37.640 | −7.172 | 1.00 | 30.81 | A | C |
| ATOM | 1415 | OD1 | ASN | A | 808 | 5.515 | 37.816 | −6.054 | 1.00 | 34.30 | A | O |
| ATOM | 1416 | ND2 | ASN | A | 808 | 6.503 | 38.628 | −7.897 | 1.00 | 30.27 | A | N |
| ATOM | 1417 | C | ASN | A | 808 | 5.800 | 34.954 | −9.919 | 1.00 | 31.26 | A | C |
| ATOM | 1418 | O | ASN | A | 808 | 6.936 | 34.847 | −10.384 | 1.00 | 30.54 | A | O |
| ATOM | 1419 | N | TYR | A | 809 | 4.895 | 33.988 | −10.023 | 1.00 | 30.03 | A | N |
| ATOM | 1420 | CA | TYR | A | 809 | 5.208 | 32.783 | −10.778 | 1.00 | 30.89 | A | C |
| ATOM | 1421 | CB | TYR | A | 809 | 4.741 | 31.525 | −10.043 | 1.00 | 28.06 | A | C |
| ATOM | 1422 | CG | TYR | A | 809 | 5.554 | 31.230 | −8.792 | 1.00 | 26.14 | A | C |
| ATOM | 1423 | CD1 | TYR | A | 809 | 5.292 | 31.899 | −7.594 | 1.00 | 24.58 | A | C |
| ATOM | 1424 | CE1 | TYR | A | 809 | 6.060 | 31.665 | −6.454 | 1.00 | 22.36 | A | C |
| ATOM | 1425 | CD2 | TYR | A | 809 | 6.614 | 30.314 | −8.819 | 1.00 | 23.89 | A | C |
| ATOM | 1426 | CE2 | TYR | A | 809 | 7.395 | 30.078 | −7.681 | 1.00 | 23.18 | A | C |
| ATOM | 1427 | CZ | TYR | A | 809 | 7.109 | 30.759 | −6.500 | 1.00 | 22.75 | A | C |
| ATOM | 1428 | OH | TYR | A | 809 | 7.862 | 30.545 | −5.356 | 1.00 | 21.98 | A | O |
| ATOM | 1429 | C | TYR | A | 809 | 4.501 | 32.957 | −12.121 | 1.00 | 31.30 | A | C |
| ATOM | 1430 | O | TYR | A | 809 | 3.275 | 32.937 | −12.207 | 1.00 | 32.33 | A | O |
| ATOM | 1431 | N | ILE | A | 810 | 5.308 | 33.154 | −13.156 | 1.00 | 34.61 | A | N |
| ATOM | 1432 | CA | ILE | A | 810 | 4.833 | 33.397 | −14.507 | 1.00 | 36.63 | A | C |
| ATOM | 1433 | GB | ILE | A | 810 | 5.886 | 34.213 | −15.294 | 1.00 | 37.90 | A | C |
| ATOM | 1434 | CG2 | ILE | A | 810 | 5.317 | 34.674 | −16.630 | 1.00 | 39.20 | A | C |
| ATOM | 1435 | CG1 | ILE | A | 810 | 6.325 | 35.425 | −14.461 | 1.00 | 37.44 | A | C |
| ATOM | 1436 | CD1 | ILE | A | 810 | 5.207 | 36.398 | −14.139 | 1.00 | 38.11 | A | C |
| ATOM | 1437 | C | ILE | A | 810 | 4.510 | 32.130 | −15.278 | 1.00 | 38.89 | A | C |
| ATOM | 1438 | O | ILE | A | 810 | 5.275 | 31.164 | −15.271 | 1.00 | 36.85 | A | O |
| ATOM | 1439 | N | VAL | A | 811 | 3.361 | 32.145 | −15.946 | 1.00 | 42.63 | A | N |
| ATOM | 1440 | CA | VAL | A | 811 | 2.931 | 31.007 | −16.738 | 1.00 | 46.63 | A | C |
| ATOM | 1441 | GB | VAL | A | 811 | 1.493 | 31.196 | −17.260 | 1.00 | 47.03 | A | C |
| ATOM | 1442 | CG1 | VAL | A | 811 | 1.116 | 30.042 | −18.171 | 1.00 | 48.48 | A | C |
| ATOM | 1443 | CG2 | VAL | A | 811 | 0.524 | 31.278 | −16.093 | 1.00 | 48.85 | A | C |
| ATOM | 1444 | C | VAL | A | 811 | 3.873 | 30.857 | −17.920 | 1.00 | 48.78 | A | C |
| ATOM | 1445 | O | VAL | A | 811 | 4.120 | 31.810 | −18.662 | 1.00 | 50.28 | A | O |
| ATOM | 1446 | N | LYS | A | 812 | 4.409 | 29.657 | −18.079 | 1.00 | 51.39 | A | N |
| ATOM | 1447 | CA | LYS | A | 812 | 5.326 | 29.360 | −19.166 | 1.00 | 53.36 | A | C |
| ATOM | 1448 | GB | LYS | A | 812 | 6.770 | 29.452 | −18.663 | 1.00 | 55.01 | A | C |
| ATOM | 1449 | CG | LYS | A | 812 | 7.839 | 29.521 | −19.749 | 1.00 | 57.32 | A | C |
| ATOM | 1450 | CD | LYS | A | 812 | 8.019 | 28.195 | −20.473 | 1.00 | 58.28 | A | C |
| ATOM | 1451 | CE | LYS | A | 812 | 9.203 | 28.254 | −21.435 | 1.00 | 59.30 | A | C |
| ATOM | 1452 | NZ | LYS | A | 812 | 9.394 | 26.975 | −22.176 | 1.00 | 59.87 | A | N |
| ATOM | 1453 | C | LYS | A | 812 | 5.007 | 27.948 | −19.655 | 1.00 | 53.67 | A | C |
| ATOM | 1454 | O | LYS | A | 812 | 5.557 | 26.964 | −19.161 | 1.00 | 53.46 | A | O |
| ATOM | 1455 | N | GLY | A | 813 | 4.097 | 27.859 | −20.620 | 1.00 | 54.01 | A | N |
| ATOM | 1456 | CA | GLY | A | 813 | 3.714 | 26.565 | −21.155 | 1.00 | 54.27 | A | C |
| ATOM | 1457 | C | GLY | A | 813 | 2.869 | 25.772 | −20.178 | 1.00 | 54.01 | A | C |
| ATOM | 1458 | O | GLY | A | 813 | 1.983 | 26.320 | −19.521 | 1.00 | 53.87 | A | O |
| ATOM | 1459 | N | ALA | A | 815 | 3.900 | 25.424 | −17.091 | 1.00 | 45.88 | A | N |
| ATOM | 1460 | CA | ALA | A | 815 | 4.825 | 25.681 | −15.993 | 1.00 | 45.35 | A | C |
| ATOM | 1461 | GB | ALA | A | 815 | 6.262 | 25.480 | −16.465 | 1.00 | 44.67 | A | C |
| ATOM | 1462 | C | ALA | A | 815 | 4.651 | 27.093 | −15.442 | 1.00 | 43.98 | A | C |
| ATOM | 1463 | O | ALA | A | 815 | 4.312 | 28.021 | −16.176 | 1.00 | 44.54 | A | O |
| ATOM | 1464 | N | ARG | A | 816 | 4.881 | 27.243 | −14.141 | 1.00 | 41.55 | A | N |
| ATOM | 1465 | CA | ARG | A | 816 | 4.773 | 28.536 | −13.467 | 1.00 | 38.90 | A | C |
| ATOM | 1466 | GB | ARG | A | 816 | 3.720 | 28.471 | −12.364 | 1.00 | 40.96 | A | C |
| ATOM | 1467 | GG | ARG | A | 816 | 2.365 | 27.986 | −12.832 | 1.00 | 44.60 | A | C |
| ATOM | 1468 | CD | ARG | A | 816 | 1.383 | 29.124 | −12.971 | 1.00 | 44.97 | A | C |
| ATOM | 1469 | NE | ARG | A | 816 | 0.455 | 28.681 | −13.619 | 1.00 | 49.11 | A | N |
| ATOM | 1470 | CZ | ARG | A | 816 | −0.948 | 29.414 | −13.707 | 1.00 | 49.56 | A | C |
| ATOM | 1471 | NH1 | ARG | A | 816 | 0.979 | 30.632 | −13.180 | 1.00 | 51.06 | A | N |
| ATOM | 1472 | NH2 | ARG | A | 816 | −2.015 | 28.935 | −14.333 | 1.00 | 50.10 | A | N |
| ATOM | 1473 | C | ARG | A | 816 | 6.147 | 28.774 | −12.857 | 1.00 | 35.77 | A | C |
| ATOM | 1474 | O | ARG | A | 816 | 6.527 | 28.108 | −11.896 | 1.00 | 33.12 | A | O |
| ATOM | 1475 | N | LEU | A | 817 | 6.891 | 29.720 | −13.416 | 1.00 | 32.40 | A | N |
| ATOM | 1476 | CA | LEU | A | 817 | 8.238 | 29.986 | −12.938 | 1.00 | 31.23 | A | C |
| ATOM | 1477 | GB | LEU | A | 817 | 9.240 | 29.758 | −14.083 | 1.00 | 31.36 | A | C |
| ATOM | 1478 | CG | LEU | A | 817 | 9.212 | 28.378 | −14.763 | 1.00 | 33.58 | A | C |
| ATOM | 1479 | CD1 | LEU | A | 817 | 10.146 | 28.371 | −15.967 | 1.00 | 33.07 | A | C |
| ATOM | 1480 | CD2 | LEU | A | 817 | 9.615 | 27.303 | −13.763 | 1.00 | 34.77 | A | C |
| ATOM | 1481 | C | LEU | A | 817 | 8.436 | 31.384 | −12.372 | 1.00 | 28.71 | A | C |
| ATOM | 1482 | O | LEU | A | 817 | 7.863 | 32.358 | −12.872 | 1.00 | 29.58 | A | O |
| ATOM | 1483 | N | PRO | A | 818 | 9.245 | 31.497 | −11.307 | 1.00 | 25.67 | A | N |
| ATOM | 1484 | CD | PRO | A | 818 | 9.855 | 30.390 | −10.545 | 1.00 | 27.34 | A | C |
| ATOM | 1485 | CA | PRO | A | 818 | 9.524 | 32.795 | −10.676 | 1.00 | 25.90 | A | C |
| ATOM | 1486 | GB | PRO | A | 818 | 10.051 | 32.392 | −9.297 | 1.00 | 26.91 | A | C |
| ATOM | 1487 | CG | PRO | A | 818 | 10.791 | 31.111 | −9.595 | 1.00 | 28.21 | A | C |
| ATOM | 1488 | C | PRO | A | 818 | 10.575 | 33.504 | −11.534 | 1.00 | 24.30 | A | C |
| ATOM | 1489 | O | PRO | A | 818 | 11.720 | 33.666 | −11.135 | 1.00 | 24.57 | A | O |
| ATOM | 1490 | N | VAL | A | 819 | 10.165 | 33.924 | −12.723 | 1.00 | 25.06 | A | N |
| ATOM | 1491 | CA | VAL | A | 819 | 11.068 | 34.558 | −13.678 | 1.00 | 25.38 | A | C |
| ATOM | 1492 | CB | VAL | A | 819 | 10.275 | 35.022 | −14.932 | 1.00 | 27.98 | A | C |
| ATOM | 1493 | CG1 | VAL | A | 819 | 11.174 | 35.784 | −15.877 | 1.00 | 29.83 | A | C |
| ATOM | 1494 | CG2 | VAL | A | 819 | 9.695 | 33.800 | −15.648 | 1.00 | 27.52 | A | C |
| ATOM | 1495 | C | VAL | A | 819 | 11.975 | 35.695 | −13.192 | 1.00 | 22.75 | A | C |
| ATOM | 1496 | O | VAL | A | 819 | 13.153 | 35.725 | −13.544 | 1.00 | 21.93 | A | O |
| ATOM | 1497 | N | LYS | A | 820 | 11.457 | 36.614 | −12.383 | 1.00 | 23.24 | A | N |
| ATOM | 1498 | CA | LYS | A | 820 | 12.295 | 37.726 | −11.917 | 1.00 | 23.65 | A | C |
| ATOM | 1499 | CB | LYS | A | 820 | 11.446 | 38.787 | −11.219 | 1.00 | 22.78 | A | C |
| ATOM | 1500 | CG | LYS | A | 820 | 10.681 | 39.690 | −12.170 | 1.00 | 23.46 | A | C |
| ATOM | 1501 | CD | LYS | A | 820 | 10.028 | 40.860 | −11.436 | 1.00 | 26.30 | A | C |
| ATOM | 1502 | CE | LYS | A | 820 | 9.266 | 41.757 | −12.406 | 1.00 | 28.19 | A | C |
| ATOM | 1503 | NZ | LYS | A | 820 | 8.786 | 43.009 | −11.752 | 1.00 | 29.14 | A | N |
| ATOM | 1504 | C | LYS | A | 820 | 13.455 | 37.314 | −11.007 | 1.00 | 20.98 | A | C |
| ATOM | 1505 | O | LYS | A | 820 | 14.339 | 38.124 | −10.703 | 1.00 | 21.94 | A | O |
| ATOM | 1506 | N | TRP | A | 821 | 13.447 | 36.061 | −10.576 | 1.00 | 21.43 | A | N |
| ATOM | 1507 | CA | TRP | A | 821 | 14.501 | 35.531 | −9.708 | 1.00 | 21.95 | A | C |
| ATOM | 1508 | CB | TRP | A | 821 | 13.877 | 34.771 | −8.526 | 1.00 | 20.40 | A | C |
| ATOM | 1509 | CG | TRP | A | 821 | 13.355 | 35.651 | −7.425 | 1.00 | 21.20 | A | C |
| ATOM | 1510 | CD2 | TRP | A | 821 | 12.089 | 36.327 | −7.388 | 1.00 | 22.91 | A | C |
| ATOM | 1511 | CE2 | TRP | A | 821 | 12.067 | 37.109 | −6.203 | 1.00 | 20.76 | A | C |
| ATOM | 1512 | CE3 | TRP | A | 821 | 10.976 | 36.354 | −8.237 | 1.00 | 22.76 | A | C |
| ATOM | 1513 | CD1 | TRP | A | 821 | 14.025 | 36.029 | −6.292 | 1.00 | 21.77 | A | C |
| ATOM | 1514 | NE1 | TRP | A | 821 | 13.255 | 36.905 | −5.555 | 1.00 | 20.97 | A | N |
| ATOM | 1515 | CZ2 | TRP | A | 821 | 10.973 | 37.910 | −5.851 | 1.00 | 22.45 | A | C |
| ATOM | 1516 | CZ3 | TRP | A | 821 | 9.883 | 37.153 | −7.887 | 1.00 | 23.18 | A | C |
| ATOM | 1517 | CH2 | TRP | A | 821 | 9.893 | 37.922 | −6.700 | 1.00 | 21.21 | A | C |
| ATOM | 1518 | C | TRP | A | 821 | 15.426 | 34.576 | −10.459 | 1.00 | 25.30 | A | C |
| ATOM | 1519 | O | TRP | A | 821 | 16.453 | 34.148 | −9.926 | 1.00 | 24.27 | A | O |
| ATOM | 1520 | N | MET | A | 822 | 15.083 | 34.258 | −11.704 | 1.00 | 25.46 | A | N |
| ATOM | 1521 | CA | MET | A | 822 | 15.868 | 33.284 | −12.451 | 1.00 | 26.35 | A | C |
| ATOM | 1522 | CB | MET | A | 822 | 14.923 | 32.440 | −13.321 | 1.00 | 26.29 | A | C |
| ATOM | 1523 | CG | MET | A | 822 | 13.807 | 31.770 | −12.531 | 1.00 | 29.03 | A | C |
| ATOM | 1524 | SD | MET | A | 822 | 12.536 | 31.000 | −13.580 | 1.00 | 33.03 | A | S |
| ATOM | 1525 | CE | MET | A | 822 | 13.508 | 29.730 | −14.402 | 1.00 | 34.78 | A | C |
| ATOM | 1526 | C | MET | A | 822 | 17.028 | 33.792 | −13.298 | 1.00 | 26.11 | A | C |
| ATOM | 1527 | O | MET | A | 822 | 16.946 | 34.838 | −13.942 | 1.00 | 27.94 | A | O |
| ATOM | 1528 | N | ALA | A | 823 | 18.108 | 33.020 | −13.299 | 1.00 | 27.22 | A | N |
| ATOM | 1529 | CA | ALA | A | 823 | 19.293 | 33.356 | −14.075 | 1.00 | 29.26 | A | C |
| ATOM | 1530 | CB | ALA | A | 823 | 20.443 | 32.430 | −13.706 | 1.00 | 30.03 | A | C |
| ATOM | 1531 | C | ALA | A | 823 | 18.943 | 33.197 | −15.555 | 1.00 | 30.96 | A | C |
| ATOM | 1532 | O | ALA | A | 823 | 18.088 | 32.388 | −15.913 | 1.00 | 30.01 | A | O |
| ATOM | 1533 | N | PRO | A | 824 | 19.606 | 33.964 | −16.429 | 1.00 | 32.64 | A | N |
| ATOM | 1534 | CD | PRO | A | 824 | 20.732 | 34.872 | −16.162 | 1.00 | 33.05 | A | C |
| ATOM | 1535 | CA | PRO | A | 824 | 19.334 | 33.886 | −17.868 | 1.00 | 36.11 | A | C |
| ATOM | 1536 | CB | PRO | A | 824 | 20.350 | 34.861 | −18.464 | 1.00 | 36.00 | A | C |
| ATOM | 1537 | CG | PRO | A | 824 | 21.480 | 34.831 | −17.473 | 1.00 | 37.14 | A | C |
| ATOM | 1538 | C | PRO | A | 824 | 19.430 | 32.480 | −18.469 | 1.00 | 36.52 | A | C |
| ATOM | 1539 | O | PRO | A | 824 | 18.550 | 32.074 | −19.219 | 1.00 | 37.88 | A | O |
| ATOM | 1540 | N | GLU | A | 825 | 20.482 | 31.738 | −18.138 | 1.00 | 37.16 | A | C |
| ATOM | 1541 | CA | GLU | A | 825 | 20.635 | 30.387 | −18.679 | 1.0.0 | 37.90 | A | C |
| ATOM | 1542 | CB | GLU | A | 825 | 21.939 | 29.742 | −18.191 | 1.00 | 37.37 | A | C |
| ATOM | 1543 | CG | GLU | A | 825 | 21.981 | 29.490 | −16.696 | 1.00 | 38.94 | A | C |
| ATOM | 1544 | CD | GLU | A | 825 | 22.734 | 30.564 | −15.943 | 1.00 | 39.03 | A | C |
| ATOM | 1545 | OE1 | GLU | A | 825 | 22.632 | 31.753 | −16.321 | 1.00 | 35.79 | A | O |
| ATOM | 1546 | OE2 | GLU | A | 825 | 23.425 | 30.212 | −14.966 | 1.00 | 40.40 | A | O |
| ATOM | 1547 | C | GLU | A | 825 | 19.450 | 29.509 | −18.286 | 1.00 | 37.51 | A | C |
| ATOM | 1548 | O | GLU | A | 825 | 19.036 | 28.637 | −19.051 | 1.00 | 36.46 | A | O |
| ATOM | 1549 | N | SER | A | 826 | 18.905 | 29.737 | −17.090 | 1.00 | 36.70 | A | N |
| ATOM | 1550 | CA | SER | A | 826 | 17.756 | 28.968 | −16.618 | 1.00 | 36.53 | A | C |
| ATOM | 1551 | CB | SER | A | 826 | 17.510 | 29.212 | −15.122 | 1.00 | 35.67 | A | C |
| ATOM | 1552 | OG | SER | A | 826 | 18.527 | 28.619 | −14.339 | 1.00 | 32.85 | A | O |
| ATOM | 1553 | C | SER | A | 826 | 16.516 | 29.376 | −17.401 | 1.00 | 38.69 | A | C |
| ATOM | 1554 | O | SER | A | 826 | 15.623 | 28.563 | −17.644 | 1.00 | 39.70 | A | O |
| ATOM | 1555 | N | ILE | A | 827 | 16.475 | 30.646 | −17.787 | 1.00 | 39.66 | A | N |
| ATOM | 1556 | CA | ILE | A | 827 | 15.359 | 31.200 | −18.543 | 1.00 | 41.87 | A | C |
| ATOM | 1557 | CB | ILE | A | 827 | 15.288 | 32.735 | −18.374 | 1.00 | 41.39 | A | C |
| ATOM | 1558 | CG2 | ILE | A | 827 | 14.235 | 33.319 | −19.307 | 1.00 | 41.88 | A | C |
| ATOM | 1559 | CG1 | ILE | A | 827 | 14.988 | 33.088 | −16.914 | 1.00 | 40.54 | A | C |
| ATOM | 1560 | CD1 | ILE | A | 827 | 14.921 | 34.573 | −16.654 | 1.00 | 40.32 | A | C |
| ATOM | 1561 | C | ILE | A | 827 | 15.449 | 30.904 | −20.039 | 1.00 | 43.01 | A | C |
| ATOM | 1562 | O | ILE | A | 827 | 14.459 | 30.539 | −20.662 | 1.00 | 45.05 | A | O |
| ATOM | 1563 | N | PHE | A | 828 | 16.638 | 31.060 | −20.611 | 1.00 | 44.96 | A | N |
| ATOM | 1564 | CA | PHE | A | 828 | 16.816 | 30.855 | −22.045 | 1.00 | 46.18 | A | C |
| ATOM | 1565 | CB | PHE | A | 828 | 17.756 | 31.932 | −22.592 | 1.00 | 46.21 | A | C |
| ATOM | 1566 | CG | PHE | A | 828 | 17.361 | 33.323 | −22.203 | 1.00 | 45.87 | A | C |
| ATOM | 1567 | CD1 | PHE | A | 828 | 16.098 | 33.807 | −22.506 | 1.00 | 47.66 | A | C |
| ATOM | 1568 | CD2 | PHE | A | 828 | 18.242 | 34.144 | −21.516 | 1.00 | 46.78 | A | C |
| ATOM | 1569 | CE1 | PHE | A | 828 | 15.716 | 35.090 | −22.130 | 1.00 | 47.02 | A | C |
| ATOM | 1570 | CE2 | PHE | A | 828 | 17.869 | 35.427 | −21.137 | 1.00 | 45.88 | A | C |
| ATOM | 1571 | CZ | PHE | A | 828 | 16.603 | 35.899 | −21.446 | 1.00 | 46.14 | A | C |
| ATOM | 1572 | C | PHE | A | 828 | 17.304 | 29.475 | −22.481 | 1.00 | 47.41 | A | C |
| ATOM | 1573 | O | PHE | A | 828 | 16.941 | 29.001 | −23.559 | 1.00 | 46.29 | A | O |
| ATOM | 1574 | N | ASP | A | 829 | 18.121 | 28.831 | −21.653 | 1.00 | 48.48 | A | N |
| ATOM | 1575 | CA | ASP | A | 829 | 18.643 | 27.511 | −21.992 | 1.00 | 49.25 | A | C |
| ATOM | 1576 | CB | ASP | A | 829 | 20.170 | 27.502 | −21.879 | 1.00 | 50.73 | A | C |
| ATOM | 1577 | CG | ASP | A | 829 | 20.813 | 28.679 | −22.588 | 1.00 | 51.29 | A | C |
| ATOM | 1578 | OD1 | ASP | A | 829 | 20.362 | 29.018 | −23.703 | 1.00 | 51.01 | A | O |
| ATOM | 1579 | OD2 | ASP | A | 829 | 21.772 | 29.262 | −22.036 | 1.00 | 52.69 | A | O |
| ATOM | 1580 | C | ASP | A | 829 | 18.058 | 26.424 | −21.103 | 1.00 | 49.72 | A | C |
| ATOM | 1581 | O | ASP | A | 829 | 18.333 | 25.242 | −21.293 | 1.00 | 50.40 | A | O |
| ATOM | 1582 | N | CYS | A | 830 | 17.250 | 26.830 | −20.130 | 1.00 | 49.53 | A | N |
| ATOM | 1583 | CA | CYS | A | 830 | 16.631 | 25.889 | −19.212 | 1.00 | 48.08 | A | C |
| ATOM | 1584 | CB | CYS | A | 830 | 15.655 | 24.996 | −19.974 | 1.00 | 50.60 | A | C |
| ATOM | 1585 | SG | CYS | A | 830 | 14.455 | 25.950 | −20.936 | 1.00 | 56.39 | A | S |
| ATOM | 1586 | C | CYS | A | 830 | 17.695 | 25.051 | −18.499 | 1.00 | 45.33 | A | C |
| ATOM | 1587 | O | CYS | A | 830 | 17.523 | 23.855 | −18.258 | 1.00 | 45.13 | A | O |
| ATOM | 1588 | N | VAL | A | 831 | 18.806 | 25.703 | −18.181 | 1.00 | 42.08 | A | N |
| ATOM | 1589 | CA | VAL | A | 831 | 19.907 | 25.069 | −17.477 | 1.00 | 40.31 | A | C |
| ATOM | 1590 | CB | VAL | A | 831 | 21.270 | 25.533 | −18.031 | 1.00 | 40.86 | A | C |
| ATOM | 1591 | CG1 | VAL | A | 831 | 22.391 | 25.055 | −17.123 | 1.00 | 41.87 | A | C |
| ATOM | 1592 | CG2 | VAL | A | 831 | 21.470 | 24.990 | −19.439 | 1.00 | 41.81 | A | C |
| ATOM | 1593 | C | VAL | A | 831 | 19.811 | 25.486 | −16.012 | 1.00 | 39.46 | A | C |
| ATOM | 1594 | O | VAL | A | 831 | 19.648 | 26.668 | −15.707 | 1.00 | 36.83 | A | O |
| ATOM | 1595 | N | TYR | A | 832 | 19.904 | 24.514 | −15.115 | 1.00 | 38.97 | A | N |
| ATOM | 1596 | CA | TYR | A | 832 | 19.834 | 24.784 | −13.683 | 1.00 | 38.57 | A | C |
| ATOM | 1597 | CB | TYR | A | 832 | 18.562 | 24.185 | −13.078 | 1.00 | 40.68 | A | C |
| ATOM | 1598 | CG | TYR | A | 832 | 17.279 | 24.884 | −13.470 | 1.00 | 44.99 | A | C |
| ATOM | 1599 | CD1 | TYR | A | 832 | 16.638 | 24.592 | −14.671 | 1.00 | 47.10 | A | C |
| ATOM | 1600 | CE1 | TYR | A | 832 | 15.433 | 25.209 | −15.013 | 1.00 | 48.30 | A | C |
| ATOM | 1601 | CD2 | TYR | A | 832 | 16.689 | 25.819 | −12.617 | 1.00 | 47.41 | A | C |
| ATOM | 1602 | CE2 | TYR | A | 832 | 15.490 | 26.441 | −12.946 | 1.00 | 48.07 | A | C |
| ATOM | 1603 | CZ | TYR | A | 832 | 14.865 | 26.130 | −14.142 | 1.00 | 48.95 | A | C |
| ATOM | 1604 | OH | TYR | A | 832 | 13.660 | 26.721 | −14.447 | 1.00 | 51.19 | A | O |
| ATOM | 1605 | C | TYR | A | 832 | 21.041 | 24.194 | −12.978 | 1.00 | 36.09 | A | C |
| ATOM | 1606 | O | TYR | A | 832 | 21.223 | 22.982 | −12.965 | 1.00 | 36.46 | A | O |
| ATOM | 1607 | N | THR | A | 833 | 21.859 | 25.058 | −12.390 | 1.00 | 34.91 | A | N |
| ATOM | 1608 | CA | THR | A | 833 | 23.055 | 24.623 | −11.676 | 1.00 | 34.70 | A | C |
| ATOM | 1609 | CB | THR | A | 833 | 24.316 | 24.832 | −12.518 | 1.00 | 33.56 | A | C |
| ATOM | 1610 | OG1 | THR | A | 833 | 24.607 | 26.241 | −12.578 | 1.00 | 34.75 | A | O |
| ATOM | 1611 | CG2 | THR | A | 833 | 24.108 | 24.307 | −13.937 | 1.00 | 36.16 | A | C |
| ATOM | 1612 | C | THR | A | 833 | 23.211 | 25.455 | −10.411 | 1.00 | 34.07 | A | C |
| ATOM | 1613 | O | THR | A | 833 | 22.394 | 26.334 | −10.135 | 1.00 | 33.13 | A | O |
| ATOM | 1614 | N | VAL | A | 834 | 24.272 | 25.188 | −9.655 | 1.00 | 32.64 | A | N |
| ATOM | 1615 | CA | VAL | A | 834 | 24.533 | 25.939 | −8.431 | 1.00 | 32.94 | A | C |
| ATOM | 1616 | CB | VAL | A | 834 | 25.758 | 25.365 | −7.687 | 1.00 | 34.05 | A | C |
| ATOM | 1617 | CG1 | VAL | A | 834 | 26.125 | 26.249 | −6.491 | 1.00 | 34.80 | A | C |
| ATOM | 1618 | CG2 | VAL | A | 834 | 25.441 | 23.956 | −7.211 | 1.00 | 33.84 | A | C |
| ATOM | 1619 | C | VAL | A | 834 | 24.764 | 27.415 | −8.756 | 1.00 | 32.61 | A | C |
| ATOM | 1620 | O | VAL | A | 834 | 24.373 | 28.294 | −7.986 | 1.00 | 30.61 | A | O |
| ATOM | 1621 | N | GLN | A | 835 | 25.394 | 27.683 | −9.901 | 1.00 | 31.06 | A | N |
| ATOM | 1622 | CA | GLN | A | 835 | 25.662 | 29.050 | −10.327 | 1.00 | 30.91 | A | C |
| ATOM | 1623 | CB | GLN | A | 835 | 26.550 | 29.071 | −11.580 | 1.00 | 33.73 | A | C |
| ATOM | 1624 | CG | GLN | A | 835 | 27.897 | 28.390 | −11.411 | 1.00 | 38.14 | A | C |
| ATOM | 1625 | CD | GLN | A | 835 | 27.789 | 26.880 | −11.390 | 1.00 | 41.64 | A | C |
| ATOM | 1626 | OE1 | GLN | A | 835 | 27.435 | 26.252 | −12.392 | 1.00 | 44.54 | A | O |
| ATOM | 1627 | NE2 | GLN | A | 835 | 28.086 | 26.288 | −10.245 | 1.00 | 43.71 | A | N |
| ATOM | 1628 | C | GLN | A | 835 | 24.354 | 29.782 | −10.619 | 1.00 | 29.24 | A | C |
| ATOM | 1629 | O | GLN | A | 835 | 24.274 | 31.005 | −10.475 | 1.00 | 28.16 | A | O |
| ATOM | 1630 | N | SER | A | 836 | 23.343 | 29.033 | −11.046 | 1.00 | 26.36 | A | N |
| ATOM | 1631 | CA | SER | A | 836 | 22.027 | 29.600 | −11.322 | 1.00 | 26.94 | A | C |
| ATOM | 1632 | CB | SER | A | 836 | 21.100 | 28.552 | −11.946 | 1.00 | 28.83 | A | C |
| ATOM | 1633 | OG | SER | A | 836 | 21.619 | 28.072 | −13.177 | 1.00 | 39.97 | A | O |
| ATOM | 1634 | C | SER | A | 836 | 21.423 | 30.085 | −9.997 | 1.00 | 25.54 | A | C |
| ATOM | 1635 | O | SER | A | 836 | 20.822 | 31.160 | −9.944 | 1.00 | 23.88 | A | O |
| ATOM | 1636 | N | ASP | A | 837 | 21.590 | 29.294 | −8.937 | 1.00 | 23.44 | A | N |
| ATOM | 1637 | CA | ASP | A | 837 | 21.069 | 29.666 | −7.612 | 1.00 | 22.87 | A | C |
| ATOM | 1638 | CB | ASP | A | 837 | 21.223 | 28.522 | −6.587 | 1.00 | 21.76 | A | C |
| ATOM | 1639 | CG | ASP | A | 837 | 20.251 | 27.353 | −6.824 | 1.00 | 26.24 | A | C |
| ATOM | 1640 | OD1 | ASP | A | 837 | 19.127 | 27.572 | −7.326 | 1.00 | 22.61 | A | O |
| ATOM | 1641 | OD2 | ASP | A | 837 | 20.614 | 26.205 | −6.475 | 1.00 | 26.73 | A | O |
| ATOM | 1642 | C | ASP | A | 837 | 21.806 | 30.887 | −7.072 | 1.00 | 20.33 | A | C |
| ATOM | 1643 | O | ASP | A | 837 | 21.258 | 31.649 | −6.281 | 1.00 | 21.19 | A | O |
| ATOM | 1644 | N | VAL | A | 838 | 23.062 | 31.059 | −7.479 | 1.00 | 20.59 | A | N |
| ATOM | 1645 | CA | VAL | A | 838 | 23.857 | 32.206 | −7.039 | 1.00 | 19.23 | A | C |
| ATOM | 1646 | CB | VAL | A | 838 | 25.322 | 32.102 | −7.537 | 1.00 | 19.55 | A | C |
| ATOM | 1647 | CG1 | VAL | A | 838 | 26.057 | 33.428 | −7.305 | 1.00 | 20.98 | A | C |
| ATOM | 1648 | CG2 | VAL | A | 838 | 26.036 | 30.964 | −6.804 | 1.00 | 21.35 | A | C |
| ATOM | 1649 | C | VAL | A | 838 | 23.238 | 33.500 | −7.581 | 1.00 | 20.52 | A | C |
| ATOM | 1650 | O | VAL | A | 838 | 23.179 | 34.517 | −6.887 | 1.00 | 19.90 | A | O |
| ATOM | 1651 | N | TRP | A | 839 | 22.810 | 33.460 | −8.836 | 1.00 | 18.46 | A | N |
| ATOM | 1652 | CA | TRP | A | 839 | 22.159 | 34.609 | −9.453 | 1.00 | 21.09 | A | C |
| ATOM | 1653 | CB | TRP | A | 839 | 21.728 | 34.268 | −10.889 | 1.00 | 20.07 | A | C |
| ATOM | 1654 | CG | TRP | A | 839 | 20.884 | 35.332 | −11.573 | 1.00 | 21.41 | A | C |
| ATOM | 1655 | CD2 | TRP | A | 839 | 21.289 | 36.193 | −12.646 | 1.00 | 24.44 | A | C |
| ATOM | 1656 | CE2 | TRP | A | 839 | 20.178 | 37.005 | −12.972 | 1.00 | 25.22 | A | C |
| ATOM | 1657 | CE3 | TRP | A | 839 | 22.482 | 36.357 | −13.363 | 1.00 | 25.77 | A | C |
| ATOM | 1658 | CD1 | TRP | A | 839 | 19.585 | 35.649 | −11.302 | 1.00 | 23.59 | A | C |
| ATOM | 1659 | NE1 | TRP | A | 839 | 19.152 | 36.651 | −12.136 | 1.00 | 25.28 | A | N |
| ATOM | 1660 | CZ2 | TRP | A | 839 | 20.225 | 37.971 | −13.987 | 1.00 | 28.13 | A | C |
| ATOM | 1661 | CZ3 | TRP | A | 839 | 22.528 | 37.322 | −14.377 | 1.00 | 27.43 | A | C |
| ATOM | 1662 | CH2 | TRP | A | 839 | 21.404 | 38.113 | −14.675 | 1.00 | 25.19 | A | C |
| ATOM | 1663 | C | TRP | A | 839 | 20.928 | 34.953 | −8.611 | 1.00 | 21.19 | A | C |
| ATOM | 1664 | O | TRP | A | 839 | 20.727 | 36.109 | −8.224 | 1.00 | 20.89 | A | O |
| ATOM | 1665 | N | SER | A | 840 | 20.121 | 33.938 | −8.319 | 1.00 | 21.96 | A | N |
| ATOM | 1666 | CA | SER | A | 840 | 18.907 | 34.122 | −7.528 | 1.00 | 21.65 | A | C |
| ATOM | 1667 | CS | SER | A | 840 | 18.155 | 32.800 | −7.383 | 1.00 | 22.22 | A | C |
| ATOM | 1668 | OG | SER | A | 840 | 17.702 | 32.334 | −8.642 | 1.00 | 25.05 | A | C |
| ATOM | 1669 | C | SER | A | 840 | 19.235 | 34.678 | −6.147 | 1.00 | 21.66 | A | C |
| ATOM | 1670 | O | SER | A | 840 | 18.483 | 35.483 | −5.603 | 1.00 | 20.11 | A | O |
| ATOM | 1671 | N | TYR | A | 841 | 20.350 | 34.231 | −5.583 | 1.00 | 21.97 | A | N |
| ATOM | 1672 | CA | TYR | A | 841 | 20.773 | 34.708 | −4.276 | 1.00 | 20.78 | A | C |
| ATOM | 1673 | CB | TYR | A | 841 | 22.023 | 33.967 | −3.817 | 1.00 | 20.77 | A | C |
| ATOM | 1674 | CG | TYR | A | 841 | 22.572 | 34.515 | −2.519 | 1.00 | 21.90 | A | C |
| ATOM | 1675 | CD1 | TYR | A | 841 | 22.030 | 34.129 | −1.296 | 1.00 | 19.00 | A | C |
| ATOM | 1676 | CE1 | TYR | A | 841 | 22.514 | 34.676 | −0.089 | 2.00 | 19.51 | A | C |
| ATOM | 1677 | CD2 | TYR | A | 841 | 23.603 | 35.457 | −2.522 | 1.00 | 19.80 | A | C |
| ATOM | 1678 | CE2 | TYR | A | 841 | 24.085 | 36.009 | −1.337 | 1.00 | 20.67 | A | C |
| ATOM | 1679 | CZT | YR | A | 841 | 23.541 | 35.610 | −0.128 | 1.00 | 19.20 | A | C |
| ATOM | 1680 | OH | TYR | A | 841 | 24.055 | 36.136 | 1.044 | 1.00 | 23.37 | A | O |
| ATOM | 1681 | C | TYR | A | 841 | 21.058 | 36.209 | −4.343 | 1.00 | 21.29 | A | C |
| ATOM | 1682 | O | TYR | A | 841 | 20.896 | 36.916 | −3.354 | 1.00 | 21.31 | A | O |
| ATOM | 1683 | N | GLY | A | 842 | 21.495 | 36.681 | −5.509 | 1.00 | 21.07 | A | N |
| ATOM | 1684 | CA | GLY | A | 842 | 21.780 | 38.097 | −5.699 | 1.00 | 21.51 | A | C |
| ATOM | 1685 | C | GLY | A | 842 | 20.483 | 38.892 | −5.686 | 1.00 | 21.46 | A | C |
| ATOM | 1686 | O | GLY | A | 842 | 20.433 | 40.003 | −5.155 | 1.00 | 21.16 | A | O |
| ATOM | 1687 | N | ILE | A | 843 | 19.437 | 38.318 | −6.275 | 1.00 | 19.90 | A | N |
| ATOM | 1688 | CA | ILE | A | 843 | 18.111 | 38.949 | −6.300 | 1.00 | 19.14 | A | C |
| ATOM | 1689 | CB | ILE | A | 843 | 17.124 | 38.152 | −7.202 | 1.00 | 19.48 | A | C |
| ATOM | 1690 | CG2 | ILE | A | 843 | 15.717 | 38.768 | −7.136 | 1.00 | 19.99 | A | C |
| ATOM | 1691 | CG1 | ILE | A | 843 | 17.643 | 38.126 | −8.654 | 1.00 | 19.93 | A | C |
| ATOM | 1692 | CD1 | ILE | A | 843 | 17.690 | 39.496 | −9.345 | 1.00 | 21.14 | A | C |
| ATOM | 1693 | C | ILE | A | 843 | 17.577 | 38.973 | −4.852 | 1.00 | 20.59 | A | C |
| ATOM | 1694 | O | ILE | A | 843 | 16.952 | 39.942 | −4.432 | 1.00 | 18.68 | A | O |
| ATOM | 1695 | N | LEU | A | 844 | 17.822 | 37.900 | −4.103 | 1.00 | 18.05 | A | N |
| ATOM | 1696 | CA | LEU | A | 844 | 17.384 | 37.832 | −2.702 | 1.00 | 18.39 | A | C |
| ATOM | 1697 | CB | LEU | A | 844 | 17.763 | 36.476 | −2.100 | 1.00 | 18.49 | A | C |
| ATOM | 1698 | CG | LEU | A | 844 | 17.341 | 35.958 | −0.707 | 1.00 | 24.74 | A | C |
| ATOM | 1699 | CD1 | LEU | A | 844 | 18.566 | 35.777 | 0.136 | 1.00 | 26.26 | A | C |
| ATOM | 1700 | CD2 | LEU | A | 844 | 16.314 | 36.842 | −0.034 | 1.00 | 20.82 | A | C |
| ATOM | 1701 | C | LEU | A | 844 | 18.064 | 38.956 | −1.920 | 1.00 | 18.40 | A | C |
| ATOM | 1702 | O | LEU | A | 844 | 17.435 | 39.597 | −1.081 | 1.00 | 18.40 | A | O |
| ATOM | 1703 | N | LEU | A | 845 | 19.349 | 39.188 | −2.189 | 1.00 | 17.22 | A | N |
| ATOM | 1704 | CA | LEU | A | 845 | 20.094 | 40.252 | −1.503 | 1.00 | 19.83 | A | C |
| ATOM | 1705 | CB | LEU | A | 845 | 21.564 | 40.286 | −1.945 | 1.00 | 21.47 | A | C |
| ATOM | 1706 | CG | LEU | A | 845 | 22.550 | 39.286 | −1.341 | 1.00 | 24.97 | A | C |
| ATOM | 1707 | CD1 | LEU | A | 845 | 23.943 | 39.522 | −1.921 | 1.00 | 25.32 | A | C |
| ATOM | 1708 | CD2 | LEU | A | 845 | 22.585 | 39.442 | 0.162 | 1.00 | 27.48 | A | C |
| ATOM | 1709 | C | LEU | A | 845 | 19.472 | 41.611 | −1.781 | 1.00 | 21.02 | A | C |
| ATOM | 1710 | O | LEU | A | 845 | 19.414 | 42.471 | −0.905 | 1.00 | 21.54 | A | O |
| ATOM | 1711 | N | TRP | A | 846 | 19.023 | 41.808 | −3.015 | 1.00 | 18.78 | A | N |
| ATOM | 1712 | CA | TRP | A | 846 | 18.386 | 43.065 | −3.387 | 1.00 | 19.24 | A | C |
| ATOM | 1713 | CB | TRP | A | 846 | 18.135 | 43.099 | −4.904 | 1.00 | 18.36 | A | C |
| ATOM | 1714 | CG | TRP | A | 846 | 17.666 | 44.427 | −5.397 | 1.00 | 19.12 | A | C |
| ATOM | 1715 | CD2 | TRP | A | 846 | 16.311 | 44.880 | −5.474 | 1.00 | 18.67 | A | C |
| ATOM | 1716 | CE2 | TRP | A | 846 | 16.339 | 46.211 | −5.943 | 1.00 | 20.25 | A | C |
| ATOM | 1717 | CE3 | TRP | A | 846 | 15.068 | 44.287 | −5.191 | 1.00 | 21.90 | A | C |
| ATOM | 1718 | CD1 | TRP | A | 846 | 18.445 | 45.470 | −5.813 | 1.00 | 18.34 | A | C |
| ATOM | 1719 | NE1 | TRP | A | 846 | 17.654 | 46.545 | −6.143 | 1.00 | 19.55 | A | N |
| ATOM | 1720 | CZ2 | TRP | A | 846 | 15.171 | 46.965 | −6.137 | 1.00 | 18.90 | A | C |
| ATOM | 1721 | CZ3 | TRP | A | 846 | 13.912 | 45.032 | −5.383 | 1.00 | 18.91 | A | C |
| ATOM | 1722 | CH2 | TRP | A | 846 | 13.972 | 46.360 | −5.852 | 1.00 | 19.95 | A | C |
| ATOM | 1723 | C | TRP | A | 846 | 17.063 | 43.196 | −2.617 | 1.00 | 20.29 | A | C |
| ATOM | 1724 | O | TRP | A | 846 | 16.687 | 44.295 | −2.197 | 1.00 | 20.60 | A | O |
| ATOM | 1725 | N | GLU | A | 847 | 16.354 | 42.084 | −2.430 | 1.00 | 18.32 | A | N |
| ATOM | 1726 | CA | GLU | A | 847 | 15.096 | 42.121 | −1.677 | 1.00 | 20.83 | A | C |
| ATOM | 1727 | CB | GLU | A | 847 | 14.388 | 40.767 | −1.690 | 1.00 | 21.03 | A | C |
| ATOM | 1728 | CG | GLU | A | 847 | 13.843 | 40.301 | −3.026 | 1.00 | 22.05 | A | C |
| ATOM | 1729 | CD | GLU | A | 847 | 13.127 | 38.968 | −2.882 | 1.00 | 21.19 | A | C |
| ATOM | 1730 | OE1 | GLU | A | 847 | 11.887 | 38.961 | −2.708 | 1.00 | 23.94 | A | O |
| ATOM | 1731 | OE2 | GLU | A | 847 | 13.812 | 37.929 | −2.917 | 1.00 | 20.13 | A | O |
| ATOM | 1732 | C | GLU | A | 847 | 15.362 | 42.482 | −0.215 | 1.00 | 20.02 | A | C |
| ATOM | 1733 | O | GLT | A | 847 | 14.621 | 43.252 | 0.392 | 1.00 | 21.93 | A | O |
| ATOM | 1734 | N | ILE | A | 848 | 16.407 | 41.891 | 0.351 | 1.00 | 20.95 | A | N |
| ATOM | 1735 | CA | ILE | A | 848 | 16.762 | 42.153 | 1.736 | 1.00 | 22.78 | A | C |
| ATOM | 1736 | GB | ILE | A | 848 | 17.938 | 41.261 | 2.178 | 1.00 | 23.55 | A | C |
| ATOM | 1737 | CG2 | ILE | A | 848 | 18.418 | 41.671 | 3.584 | 1.00 | 24.98 | A | C |
| ATOM | 1738 | CG1 | ILE | A | 848 | 17.515 | 39.794 | 2.154 | 1.00 | 19.41 | A | C |
| ATOM | 1739 | CD1 | ILE | A | 848 | 18.635 | 38.813 | 2.568 | 1.00 | 20.86 | A | C |
| ATOM | 1740 | C | ILE | A | 848 | 17.168 | 43.617 | 1.947 | 1.00 | 23.73 | A | C |
| ATOM | 1741 | O | ILE | A | 848 | 16.682 | 44.277 | 2.862 | 1.00 | 23.90 | A | O |
| ATOM | 1742 | N | PHE | A | 849 | 18.048 | 44.129 | 1.091 | 1.00 | 22.34 | A | N |
| ATOM | 1743 | CA | PHE | A | 849 | 18.513 | 45.495 | 1.269 | 1.00 | 21.50 | A | C |
| ATOM | 1744 | GB | PHE | A | 849 | 19.950 | 45.594 | 0.766 | 1.00 | 20.67 | A | C |
| ATOM | 1745 | CG | PHE | A | 849 | 20.923 | 44.848 | 1.638 | 1.00 | 24.48 | A | C |
| ATOM | 1746 | CD1 | PHE | A | 849 | 21.439 | 45.441 | 2.788 | 1.00 | 24.74 | A | C |
| ATOM | 1747 | CD2 | PHE | A | 849 | 21.251 | 43.522 | 1.361 | 1.00 | 24.69 | A | C |
| ATOM | 1748 | CE1 | PHE | A | 849 | 22.266 | 44.723 | 3.656 | 1.00 | 26.97 | A | C |
| ATOM | 1749 | CE2 | PHE | A | 849 | 22.074 | 42.790 | 2.219 | 1.00 | 26.68 | A | C |
| ATOM | 1750 | CZ | PHE | A | 849 | 22.583 | 43.393 | 3.374 | 1.00 | 28.40 | A | C |
| ATOM | 1751 | C | PHE | A | 849 | 17.627 | 46.614 | 0.735 | 1.00 | 23.57 | A | C |
| ATOM | 1752 | O | PHE | A | 849 | 17.996 | 47.789 | 0.807 | 1.00 | 25.95 | A | O |
| ATOM | 1753 | N | SER | A | 850 | 16.461 | 46.242 | 0.207 | 1.00 | 21.47 | A | N |
| ATOM | 1754 | CA | SER | A | 850 | 15.474 | 47.209 | −0.259 | 1.00 | 23.11 | A | C |
| ATOM | 1755 | CB | SER | A | 850 | 14.951 | 46.856 | −1.657 | 1.00 | 23.52 | A | C |
| ATOM | 1756 | OG | SER | A | 850 | 14.191 | 45.650 | −1.647 | 1.00 | 22.60 | A | O |
| ATOM | 1757 | C | SER | A | 850 | 14.322 | 47.099 | 0.743 | 1.00 | 23.85 | A | C |
| ATOM | 1758 | O | SER | A | 850 | 13.297 | 47.764 | 0.612 | 1.00 | 23.78 | A | O |
| ATOM | 1759 | N | LEU | A | 851 | 14.518 | 46.246 | 1.744 | 1.00 | 23.92 | A | N |
| ATOM | 1760 | CA | LEU | A | 851 | 13.508 | 45.979 | 2.767 | 1.00 | 26.63 | A | C |
| ATOM | 1761 | CB | LEU | A | 851 | 13.196 | 47.239 | 3.578 | 1.00 | 30.38 | A | C |
| ATOM | 1762 | GG | LEU | A | 851 | 14.365 | 47.768 | 4.417 | 1.00 | 31.54 | A | C |
| ATOM | 1763 | CD1 | LEU | A | 851 | 13.879 | 48.927 | 5.284 | 1.00 | 34.28 | A | C |
| ATOM | 1764 | CD2 | LEU | A | 851 | 14.934 | 46.661 | 5.292 | 1.00 | 31.71 | A | C |
| ATOM | 1765 | C | LEU | A | 851 | 12.231 | 45.397 | 2.168 | 1.00 | 26.55 | A | C |
| ATOM | 1766 | O | LEU | A | 851 | 11.109 | 45.778 | 2.536 | 1.00 | 26.87 | A | O |
| ATOM | 1767 | N | GLY | A | 852 | 12.403 | 44.475 | 1.225 | 1.00 | 26.49 | A | N |
| ATOM | 1768 | CA | GLY | A | 852 | 11.257 | 43.809 | 0.629 | 1.00 | 26.06 | A | C |
| ATOM | 1769 | C | GLY | A | 852 | 10.551 | 44.320 | −0.617 | 1.00 | 26.04 | A | C |
| ATOM | 1770 | O | GLY | A | 852 | 9.405 | 43.936 | −0.856 | 1.00 | 26.29 | A | O |
| ATOM | 1771 | N | LEU | A | 853 | 11.192 | 45.166 | −1.418 | 1.00 | 25.62 | A | N |
| ATOM | 1772 | CA | LEU | A | 853 | 10.549 | 45.638 | −2.645 | 1.00 | 25.25 | A | C |
| ATOM | 1773 | GB | LEU | A | 853 | 11.360 | 46.775 | −3.283 | 1.00 | 25.04 | A | C |
| ATOM | 1774 | GG | LEU | A | 853 | 11.482 | 48.112 | −2.542 | 1.00 | 26.96 | A | C |
| ATOM | 1775 | CD1 | LEU | A | 853 | 12.402 | 49.031 | −3.333 | 1.00 | 27.36 | A | C |
| ATOM | 1776 | CD2 | LEU | A | 853 | 10.102 | 48.751 | −2.370 | 1.00 | 28.88 | A | C |
| ATOM | 1777 | C | LEU | A | 853 | 10.472 | 44.475 | −3.645 | 1.00 | 24.36 | A | C |
| ATOM | 1778 | O | LEU | A | 853 | 11.282 | 43.558 | −3.579 | 1.00 | 23.84 | A | O |
| ATOM | 1779 | N | ASN | A | 854 | 9.501 | 44.510 | −4.562 | 1.00 | 25.24 | A | N |
| ATOM | 1780 | GA | ASN | A | 854 | 9.394 | 43.471 | −5.595 | 1.00 | 24.26 | A | C |
| ATOM | 1781 | CB | ASN | A | 854 | 8.027 | 43.536 | −6.300 | 1.00 | 27.10 | A | C |
| ATOM | 1782 | CG | ASN | A | 854 | 7.845 | 42.436 | −7.359 | 1.00 | 30.71 | A | C |
| ATOM | 1783 | OD1 | ASN | A | 854 | 7.704 | 42.724 | −8.550 | 1.00 | 33.31 | A | O |
| ATOM | 1784 | ND2 | ASN | A | 854 | 7.848 | 41.176 | −6.923 | 1.00 | 31.20 | A | N |
| ATOM | 1785 | C | ASN | A | 854 | 10.521 | 43.815 | −6.582 | 1.00 | 23.02 | A | C |
| ATOM | 1786 | O | ASN | A | 854 | 10.688 | 44.977 | −6.960 | 1.00 | 22.75 | A | O |
| ATOM | 1787 | N | PRO | A | 855 | 11.317 | 42.821 | −6.995 | 1.00 | 23.48 | A | N |
| ATOM | 1788 | CD | PRO | A | 855 | 11.317 | 41.413 | −6.547 | 1.00 | 22.75 | A | C |
| ATOM | 1789 | CA | PRO | A | 855 | 12.421 | 43.075 | −7.935 | 1.00 | 23.55 | A | C |
| ATOM | 1790 | GB | PRO | A | 855 | 13.032 | 41.686 | −8.132 | 1.00 | 22.61 | A | C |
| ATOM | 1791 | CG | PRO | A | 855 | 12.752 | 41.001 | −6.809 | 1.00 | 25.22 | A | C |
| ATOM | 1792 | C | PRO | A | 855 | 11.949 | 43.703 | −9.255 | 1.00 | 23.12 | A | C |
| ATOM | 1793 | O | PRO | A | 855 | 10.808 | 43.506 | −9.671 | 1.00 | 24.77 | A | O |
| ATOM | 1794 | N | TYR | A | 856 | 12.828 | 44.462 | −9.904 | 1.00 | 22.83 | A | N |
| ATOM | 1795 | CA | TYR | A | 856 | 12.487 | 45.116 | −11.164 | 1.00 | 24.91 | A | C |
| ATOM | 1796 | GB | TYR | A | 856 | 12.343 | 44.051 | −12.261 | 1.00 | 23.41 | A | C |
| ATOM | 1797 | CG | TYR | A | 856 | 13.591 | 43.221 | −12.451 | 1.00 | 24.72 | A | C |
| ATOM | 1798 | CD1 | TYR | A | 856 | 14.687 | 43.730 | −13.151 | 1.00 | 22.35 | A | C |
| ATOM | 1799 | CE1 | TYR | A | 856 | 15.854 | 42.997 | −13.294 | 1.00 | 22.63 | A | C |
| ATOM | 1800 | CD2 | TYR | A | 856 | 13.696 | 41.941 | −11.896 | 1.00 | 23.64 | A | C |
| ATOM | 1801 | GE2 | TYR | A | 856 | 14.862 | 41.196 | −12.035 | 1.00 | 23.41 | A | C |
| ATOM | 1802 | GZ | TYR | A | 856 | 15.938 | 41.735 | −12.735 | 1.00 | 23.19 | A | C |
| ATOM | 1803 | OH | TYR | A | 856 | 17.103 | 41.023 | −12.863 | 1.00 | 26.10 | A | O |
| ATOM | 1804 | C | TYR | A | 856 | 11.165 | 45.864 | −10.961 | 1.00 | 26.21 | A | C |
| ATOM | 1805 | O | TYR | A | 856 | 10.189 | 45.634 | −11.678 | 1.00 | 27.12 | A | O |
| ATOM | 1806 | N | PRO | A | 857 | 11.126 | 46.787 | −9.981 | 1.00 | 27.10 | A | N |
| ATOM | 1807 | CD | PRO | A | 857 | 12.281 | 47.278 | −9.207 | 1.00 | 27.99 | A | C |
| ATOM | 1808 | CA | PRO | A | 857 | 9.920 | 47.564 | −9.670 | 1.00 | 30.26 | A | C |
| ATOM | 1809 | CB | PRO | A | 857 | 10.409 | 48.566 | −8.612 | 1.00 | 30.65 | A | C |
| ATOM | 1810 | CG | PRO | A | 857 | 11.873 | 48.693 | −8.895 | 1.00 | 30.35 | A | C |
| ATOM | 1811 | C | PRO | A | 857 | 9.215 | 48.232 | −10.850 | 1.00 | 31.72 | A | C |
| ATOM | 1812 | O | PRO | A | 857 | 9.826 | 48.961 | −11.624 | 1.00 | 31.38 | A | O |
| ATOM | 1813 | N | GLY | A | 858 | 7.921 | 47.953 | −10.976 | 1.00 | 34.17 | A | N |
| ATOM | 1814 | CA | GLY | A | 858 | 7.129 | 48.530 | −12.046 | 1.00 | 37.08 | A | C |
| ATOM | 1815 | C | GLY | A | 858 | 7.333 | 47.891 | −13.409 | 1.00 | 39.30 | A | C |
| ATOM | 1816 | O | GLY | A | 858 | 6.619 | 48.222 | −14.356 | 1.00 | 40.60 | A | O |
| ATOM | 1817 | N | ILE | A | 859 | 8.298 | 46.982 | −13.520 | 1.00 | 38.93 | A | N |
| ATOM | 1818 | CA | ILE | A | 859 | 8.567 | 46.323 | −14.795 | 1.00 | 39.91 | A | C |
| ATOM | 1819 | GB | ILE | A | 859 | 10.077 | 46.062 | −14.993 | 1.00 | 40.10 | A | C |
| ATOM | 1820 | CG2 | ILE | A | 859 | 10.320 | 45.365 | −16.337 | 1.00 | 39.94 | A | C |
| ATOM | 1821 | GG1 | ILE | A | 859 | 10.842 | 47.384 | −14.936 | 1.00 | 38.90 | A | C |
| ATOM | 1822 | CD1 | ILE | A | 859 | 12.340 | 47.236 | −15.138 | 1.00 | 38.67 | A | C |
| ATOM | 1823 | C | ILE | A | 859 | 7.828 | 44.998 | −14.887 | 1.00 | 40.26 | A | C |
| ATOM | 1824 | O | ILE | A | 859 | 8.036 | 44.098 | −14.070 | 1.00 | 40.34 | A | O |
| ATOM | 1825 | N | LEU | A | 860 | 6.958 | 44.890 | −15.884 | 1.00 | 40.88 | A | N |
| ATOM | 1826 | CA | LEU | A | 860 | 6.181 | 43.678 | −16.094 | 1.00 | 41.13 | A | C |
| ATOM | 1827 | GB | LEU | A | 860 | 4.956 | 43.973 | −16.963 | 1.00 | 44.17 | A | C |
| ATOM | 1828 | CG | LEU | A | 860 | 3.917 | 44.964 | −16.432 | 1.00 | 45.61 | A | C |
| ATOM | 1829 | CD1 | LEU | A | 860 | 2.847 | 45.185 | −17.492 | 1.00 | 45.40 | A | C |
| ATOM | 1830 | CD2 | LEU | A | 860 | 3.303 | 44.431 | −15.143 | 1.00 | 46.55 | A | C |
| ATOM | 1831 | C | LEU | A | 860 | 7.035 | 42.624 | −16.779 | 1.00 | 40.77 | A | C |
| ATOM | 1832 | O | LEU | A | 860 | 8.004 | 42.947 | −17.473 | 1.00 | 40.42 | A | O |
| ATOM | 1833 | N | VAL | A | 861 | 6.678 | 41.361 | −16.575 | 1.00 | 40.50 | A | N |
| ATOM | 1834 | CA | VAL | A | 861 | 7.408 | 40.271 | −17.197 | 1.00 | 39.36 | A | C |
| ATOM | 1835 | CE | VAL | A | 861 | 7.278 | 38.969 | −16.383 | 1.00 | 39.30 | A | C |
| ATOM | 1836 | CC1 | VAL | A | 861 | 7.940 | 37.817 | −17.127 | 1.00 | 39.10 | A | C |
| ATOM | 1837 | CG2 | VAL | A | 861 | 7.924 | 39.149 | −15.015 | 1.00 | 37.93 | A | C |
| ATOM | 1838 | C | VAL | A | 861 | 6.856 | 40.042 | −18.599 | 1.00 | 39.52 | A | C |
| ATOM | 1839 | O | VAL | A | 861 | 5.700 | 39.667 | −18.769 | 1.00 | 39.07 | A | O |
| ATOM | 1840 | N | ASN | A | 862 | 7.694 | 40.295 | −19.598 | 1.00 | 40.50 | A | N |
| ATOM | 1841 | CA | ASN | A | 862 | 7.328 | 40.108 | −20.996 | 1.00 | 40.72 | A | C |
| ATOM | 1842 | GB | ASN | A | 862 | 6.300 | 41.157 | −21.444 | 1.00 | 40.69 | A | C |
| ATOM | 1843 | CG | ASN | A | 862 | 6.727 | 42.573 | −21.127 | 1.00 | 42.48 | A | C |
| ATOM | 1844 | OD1 | ASN | A | 862 | 7.854 | 42.982 | −21.413 | 1.00 | 42.17 | A | O |
| ATOM | 1845 | ND2 | ASN | A | 862 | 5.815 | 43.340 | −20.541 | 1.00 | 43.91 | A | N |
| ATOM | 1846 | C | ASN | A | 862 | 8.584 | 40.198 | −21.849 | 1.00 | 41.01 | A | C |
| ATOM | 1847 | O | ASN | A | 862 | 9.694 | 40.223 | −21.320 | 1.00 | 39.88 | A | O |
| ATOM | 1848 | N | SER | A | 863 | 6.411 | 40.249 | −23.166 | 1.00 | 41.77 | A | N |
| ATOM | 1849 | CA | SER | A | 863 | 9.540 | 40.321 | −24.084 | 1.00 | 42.40 | A | C |
| ATOM | 1850 | CB | SER | A | 863 | 9.045 | 40.542 | −25.518 | 1.00 | 44.68 | A | C |
| ATOM | 1851 | OG | SER | A | 863 | 8.265 | 39.442 | −25.954 | 1.00 | 47.68 | A | O |
| ATOM | 1852 | C | SER | A | 863 | 10.550 | 41.402 | −23.728 | 1.00 | 41.88 | A | C |
| ATOM | 1853 | O | SER | A | 863 | 11.759 | 41.169 | −23.793 | 1.00 | 41.70 | A | O |
| ATOM | 1854 | N | LYS | A | 864 | 10.066 | 42.586 | −23.366 | 1.00 | 41.97 | A | N |
| ATOM | 1855 | CA | LYS | A | 864 | 10.969 | 43.675 | −23.009 | 1.00 | 41.85 | A | C |
| ATOM | 1856 | CB | LYS | A | 864 | 10.188 | 44.941 | −22.645 | 1.00 | 44.41 | A | C |
| ATOM | 1857 | CG | LYS | A | 864 | 9.420 | 45.586 | −23.794 | 1.00 | 48.47 | A | C |
| ATOM | 1858 | CD | LYS | A | 864 | 8.173 | 44.798 | −24.169 | 1.00 | 50.60 | A | C |
| ATOM | 1859 | CE | LYS | A | 864 | 7.299 | 45.595 | −25.126 | 1.00 | 52.30 | A | C |
| ATOM | 1860 | NZ | LYS | A | 864 | 6.022 | 44.895 | −25.424 | 1.00 | 53.82 | A | N |
| ATOM | 1861 | C | LYS | A | 864 | 11.844 | 43.264 | −21.828 | 1.00 | 39.55 | A | C |
| ATOM | 1862 | O | LYS | A | 864 | 13.054 | 43.496 | −21.832 | 1.00 | 39.40 | A | O |
| ATOM | 1863 | N | PHE | A | 865 | 11.229 | 42.653 | −20.820 | 1.00 | 39.12 | A | N |
| ATOM | 1864 | CA | PHE | A | 865 | 11.966 | 42.213 | −19.638 | 1.00 | 38.06 | A | C |
| ATOM | 1865 | CB | PHE | A | 865 | 11.023 | 41.562 | −18.623 | 1.00 | 36.64 | A | C |
| ATOM | 1866 | CG | PHE | A | 865 | 11.742 | 40.908 | −17.475 | 1.00 | 35.09 | A | C |
| ATOM | 1867 | CD1 | PHE | A | 865 | 12.356 | 41.679 | −16.487 | 1.00 | 35.06 | A | C |
| ATOM | 1868 | CD2 | PHE | A | 865 | 11.854 | 39.523 | −17.408 | 1.00 | 33.09 | A | C |
| ATOM | 1869 | CE1 | PHE | A | 865 | 13.076 | 41.075 | −15.448 | 1.00 | 33.16 | A | C |
| ATOM | 1870 | CE2 | PHE | A | 865 | 12.573 | 38.909 | −16.374 | 1.00 | 33.59 | A | C |
| ATOM | 1871 | CZ | PHE | A | 865 | 13.186 | 39.687 | −15.394 | 1.00 | 33.11 | A | C |
| ATOM | 1872 | C | PHE | A | 865 | 13.062 | 41.214 | −20.003 | 1.00 | 37.01 | A | C |
| ATOM | 1873 | O | PHE | A | 865 | 14.221 | 41.384 | −19.630 | 1.00 | 36.03 | A | O |
| ATOM | 1874 | N | TYR | A | 866 | 12.679 | 40.164 | −20.721 | 1.00 | 37.56 | A | N |
| ATOM | 1875 | CA | TYR | A | 866 | 13.622 | 39.136 | −21.138 | 1.00 | 38.07 | A | C |
| ATOM | 1876 | CB | TYR | A | 866 | 12.905 | 38.068 | −21.963 | 1.00 | 37.23 | A | C |
| ATOM | 1877 | CG | TYR | A | 866 | 11.923 | 37.233 | −21.177 | 1.00 | 37.11 | A | C |
| ATOM | 1878 | CD1 | TYR | A | 866 | 10.591 | 37.133 | −21.574 | 1.00 | 37.20 | A | C |
| ATOM | 1879 | CE1 | TYR | A | 866 | 9.692 | 36.332 | −20.880 | 1.00 | 37.90 | A | C |
| ATOM | 1880 | CD2 | TYR | A | 866 | 12.333 | 36.508 | −20.057 | 1.00 | 36.89 | A | C |
| ATOM | 1881 | CE2 | TYR | A | 866 | 11.444 | 35.703 | −19.359 | 1.00 | 37.09 | A | C |
| ATOM | 1882 | CZ | TYR | A | 866 | 10.127 | 35.619 | −19.772 | 1.00 | 37.53 | A | C |
| ATOM | 1883 | OH | TYR | A | 866 | 9.242 | 34.833 | −19.074 | 1.00 | 37.38 | A | O |
| ATOM | 1884 | C | TYR | A | 866 | 14.780 | 39.717 | −21.940 | 1.00 | 38.15 | A | C |
| ATOM | 1885 | O | TYR | A | 866 | 15.923 | 39.287 | −21.786 | 1.00 | 38.37 | A | O |
| ATOM | 1886 | N | LYS | A | 867 | 14.489 | 40.697 | −22.792 | 1.00 | 39.37 | A | N |
| ATOM | 1887 | CA | LYS | A | 867 | 15.528 | 41.327 | −23.603 | 1.00 | 39.25 | A | C |
| ATOM | 1888 | CB | LYS | A | 867 | 14.915 | 42.281 | −24.635 | 1.00 | 41.07 | A | C |
| ATOM | 1889 | CC | LYS | A | 867 | 14.130 | 41.598 | −25.737 | 1.00 | 44.49 | A | C |
| ATOM | 1890 | CD | LYS | A | 867 | 13.588 | 42.618 | −26.729 | 1.00 | 47.60 | A | C |
| ATOM | 1891 | CE | LYS | A | 867 | 12.748 | 41.952 | −27.805 | 1.00 | 49.22 | A | C |
| ATOM | 1892 | NZ | LYS | A | 867 | 11.581 | 41.245 | −27.210 | 1.00 | 52.03 | A | N |
| ATOM | 1893 | C | LYS | A | 867 | 16.481 | 42.101 | −22.716 | 1.00 | 37.95 | A | C |
| ATOM | 1894 | O | LYS | A | 867 | 17.688 | 42.111 | −22.943 | 1.00 | 37.68 | A | O |
| ATOM | 1895 | N | LEU | A | 868 | 15.931 | 42.759 | −21.702 | 1.00 | 37.61 | A | N |
| ATOM | 1896 | CA | LEU | A | 868 | 16.745 | 43.532 | −20.777 | 1.00 | 37.63 | A | C |
| ATOM | 1897 | CB | LEU | A | 868 | 15.848 | 44.268 | −19.776 | 1.00 | 38.23 | A | C |
| ATOM | 1898 | CC | LEU | A | 868 | 15.087 | 45.472 | −20.334 | 1.00 | 39.69 | A | C |
| ATOM | 1899 | CD1 | LEU | A | 868 | 14.050 | 45.946 | −19.337 | 1.00 | 41.66 | A | C |
| ATOM | 1900 | CD2 | LEU | A | 868 | 16.079 | 46.582 | −20.652 | 1.00 | 42.42 | A | C |
| ATOM | 1901 | C | LEU | A | 868 | 17.729 | 42.634 | −20.034 | 1.00 | 35.67 | A | C |
| ATOM | 1902 | O | LEU | A | 868 | 18.936 | 42.846 | −20.090 | 1.00 | 35.83 | A | O |
| ATOM | 1903 | N | VAL | A | 869 | 17.213 | 41.623 | −19.347 | 1.00 | 36.69 | A | N |
| ATOM | 1904 | CA | VAL | A | 869 | 18.080 | 40.721 | −18.597 | 1.00 | 38.09 | A | C |
| ATOM | 1905 | CB | VAL | A | 869 | 17.261 | 39.621 | −17.873 | 1.00 | 39.24 | A | C |
| ATOM | 1906 | CG1 | VAL | A | 869 | 16.401 | 40.248 | −16.790 | 1.00 | 38.16 | A | C |
| ATOM | 1907 | CG2 | VAL | A | 869 | 16.389 | 38.873 | −18.863 | 1.00 | 41.19 | A | C |
| ATOM | 1908 | C | VAL | A | 869 | 19.127 | 40.066 | −19.498 | 1.00 | 38.28 | A | C |
| ATOM | 1909 | O | VAL | A | 869 | 20.300 | 39.982 | −19.138 | 1.00 | 36.50 | A | O |
| ATOM | 1910 | N | LYS | A | 870 | 18.710 | 39.617 | −20.676 | 1.00 | 38.66 | A | N |
| ATOM | 1911 | CA | LYS | A | 870 | 19.651 | 38.982 | −21.589 | 1.00 | 40.80 | A | C |
| ATOM | 1912 | CB | LYS | A | 870 | 18.917 | 38.412 | −22.807 | 1.00 | 42.63 | A | C |
| ATOM | 1913 | CO | LYS | A | 870 | 19.704 | 37.334 | −23.547 | 1.00 | 45.89 | A | C |
| ATOM | 1914 | CD | LYS | A | 870 | 18.858 | 36.665 | −24.620 | 1.00 | 48.70 | A | C |
| ATOM | 1915 | CE | LYS | A | 870 | 19.481 | 35.349 | −25.084 | 1.00 | 50.99 | A | C |
| ATOM | 1916 | NZ | LYS | A | 870 | 20.848 | 35.522 | −25.648 | 1.00 | 51.81 | A | N |
| ATOM | 1917 | C | LYS | A | 870 | 20.703 | 39.992 | −22.032 | 1.00 | 39.69 | A | C |
| ATOM | 1918 | O | LYS | A | 870 | 21.872 | 39.648 | −22.193 | 1.00 | 41.55 | A | O |
| ATOM | 1919 | N | ASP | A | 871 | 20.291 | 41.245 | −22.206 | 1.00 | 39.79 | A | N |
| ATOM | 1920 | CA | ASP | A | 871 | 21.210 | 42.294 | −22.633 | 1.00 | 39.72 | A | C |
| ATOM | 1921 | CB | ASP | A | 871 | 20.435 | 43.473 | −23.244 | 1.00 | 42.15 | A | C |
| ATOM | 1922 | CO | ASP | A | 871 | 19.689 | 43.090 | −24.521 | 1.00 | 45.25 | A | C |
| ATOM | 1923 | OD1 | ASP | A | 871 | 20.140 | 42.162 | −25.226 | 1.00 | 44.93 | A | O |
| ATOM | 1924 | OD2 | ASP | A | 871 | 18.654 | 43.724 | −24.829 | 1.00 | 47.25 | A | O |
| ATOM | 1925 | C | ASP | A | 871 | 22.149 | 42.804 | −21.537 | 1.00 | 39.23 | A | C |
| ATOM | 1926 | O | ASP | A | 871 | 23.003 | 43.646 | −21.796 | 1.00 | 39.23 | A | O |
| ATOM | 1927 | N | GLY | A | 872 | 21.993 | 42.302 | −20.314 | 1.00 | 37.91 | A | N |
| ATOM | 1928 | CA | GLY | A | 872 | 22.876 | 42.719 | −19.236 | 1.00 | 35.32 | A | C |
| ATOM | 1929 | C | GLY | A | 872 | 22.337 | 43.752 | −18.262 | 1.00 | 34.33 | A | C |
| ATOM | 1930 | O | GLY | A | 872 | 23.098 | 44.322 | −17.482 | 1.00 | 34.53 | A | O |
| ATOM | 1931 | N | TYR | A | 873 | 21.034 | 44.008 | −18.302 | 1.00 | 33.63 | A | N |
| ATOM | 1932 | CA | TYR | A | 873 | 20.441 | 44.985 | −17.391 | 1.00 | 33.06 | A | C |
| ATOM | 1933 | CB | TYR | A | 873 | 18.984 | 45.254 | −17.773 | 1.00 | 33.91 | A | C |
| ATOM | 1934 | CO | TYR | A | 873 | 18.255 | 46.135 | −16.786 | 1.00 | 35.24 | A | C |
| ATOM | 1935 | CD1 | TYR | A | 873 | 18.496 | 47.509 | −16.736 | 1.00 | 36.54 | A | C |
| ATOM | 1936 | CE1 | TYR | A | 873 | 17.849 | 48.323 | −15.802 | 1.00 | 37.89 | A | C |
| ATOM | 1937 | CD2 | TYR | A | 873 | 17.346 | 45.589 | −15.874 | 1.00 | 36.66 | A | C |
| ATOM | 1938 | CE2 | TYR | A | 873 | 16.696 | 46.391 | −14.932 | 1.00 | 35.88 | A | C |
| ATOM | 1939 | CZ | TYR | A | 873 | 16.951 | 47.755 | −14.902 | 1.00 | 38.27 | A | C |
| ATOM | 1940 | OH | TYR | A | 873 | 16.316 | 48.546 | −13.978 | 1.00 | 36.23 | A | O |
| ATOM | 1941 | C | TYR | A | 873 | 20.487 | 44.455 | −15.956 | 1.00 | 29.67 | A | C |
| ATOM | 1942 | O | TYR | A | 873 | 20.287 | 43.269 | −15.731 | 1.00 | 27.02 | A | O |
| ATOM | 1943 | N | GLN | A | 874 | 20.756 | 45.342 | −15.000 | 1.00 | 29.08 | A | N |
| ATOM | 1944 | CA | GLN | A | 874 | 20.806 | 44.983 | −13.580 | 1.00 | 29.02 | A | C |
| ATOM | 1945 | CB | GLN | A | 874 | 22.258 | 44.872 | −13.087 | 1.00 | 31.26 | A | C |
| ATOM | 1946 | CO | GLN | A | 874 | 23.008 | 43.681 | −13.678 | 1.00 | 32.68 | A | C |
| ATOM | 1947 | CD | GLN | A | 874 | 24.476 | 43.620 | −13.287 | 1.00 | 31.96 | A | C |
| ATOM | 1948 | OE1 | GLN | A | 874 | 25.202 | 42.723 | −13.725 | 1.00 | 37.30 | A | O |
| ATOM | 1949 | NE2 | GLN | A | 874 | 24.920 | 44.560 | −12.468 | 1.00 | 29.71 | A | N |
| ATOM | 1950 | C | GLN | A | 874 | 20.074 | 46.050 | −12.776 | 1.00 | 27.53 | A | C |
| ATOM | 1951 | O | GLN | A | 874 | 20.074 | 47.224 | −13.142 | 1.00 | 28.08 | A | O |
| ATOM | 1952 | N | MET | A | 875 | 19.436 | 45.634 | −11.689 | 1.00 | 27.33 | A | N |
| ATOM | 1953 | CA | MET | A | 875 | 18.706 | 46.564 | −10.833 | 1.00 | 24.49 | A | C |
| ATOM | 1954 | CB | MET | A | 875 | 17.947 | 45.811 | −9.741 | 1.00 | 22.12 | A | C |
| ATOM | 1955 | CG | MET | A | 875 | 16.757 | 45.009 | −10.200 | 1.00 | 20.05 | A | C |
| ATOM | 1956 | SD | MET | A | 875 | 15.847 | 44.386 | −8.775 | 1.00 | 22.51 | A | S |
| ATOM | 1957 | CE | MET | A | 875 | 16.689 | 42.827 | −8.532 | 1.00 | 19.64 | A | C |
| ATOM | 1958 | C | MET | A | 875 | 19.684 | 47.515 | −10.168 | 1.00 | 24.42 | A | C |
| ATOM | 1959 | O | MET | A | 875 | 20.836 | 47.158 | −9.924 | 1.00 | 24.14 | A | O |
| ATOM | 1960 | N | ALA | A | 876 | 19.215 | 48.723 | −9.869 | 1.00 | 23.99 | A | N |
| ATOM | 1961 | CA | ALA | A | 876 | 20.035 | 49.726 | −9.210 | 1.00 | 25.95 | A | C |
| ATOM | 1962 | CB | ALA | A | 876 | 19.339 | 51.093 | −9.269 | 1.00 | 26.52 | A | C |
| ATOM | 1963 | C | ALA | A | 876 | 20.282 | 49.334 | −7.757 | 1.00 | 26.67 | A | C |
| ATOM | 1964 | O | ALA | A | 876 | 19.537 | 48.544 | −7.182 | 1.00 | 26.17 | A | O |
| ATOM | 1965 | N | GLN | A | 877 | 21.332 | 49.893 | −7.168 | 1.00 | 26.33 | A | N |
| ATOM | 1966 | CA | GLN | A | 877 | 21.667 | 49.624 | −5.768 | 1.00 | 26.11 | A | C |
| ATOM | 1967 | CB | GLN | A | 877 | 22.874 | 50.478 | −5.359 | 1.00 | 28.43 | A | C |
| ATOM | 1968 | CO | GLN | A | 877 | 23.213 | 50.463 | −3.875 | 1.00 | 28.07 | A | C |
| ATOM | 1969 | CD | GLN | A | 877 | 24.475 | 51.257 | −3.570 | 1.00 | 33.40 | A | C |
| ATOM | 1970 | QE1 | GLN | A | 877 | 24.890 | 52.104 | −4.357 | 1.00 | 34.37 | A | O |
| ATOM | 1971 | NE2 | GLN | A | 877 | 25.081 | 50.994 | −2.420 | 1.00 | 32.88 | A | N |
| ATOM | 1972 | C | GLN | A | 877 | 20.462 | 49.943 | −4.875 | 1.00 | 26.20 | A | C |
| ATOM | 1973 | O | GLN | A | 877 | 19.825 | 50.987 | −5.029 | 1.00 | 28.53 | A | O |
| ATOM | 1974 | N | PRO | A | 878 | 20.126 | 49.032 | −3.945 | 1.00 | 25.80 | A | N |
| ATOM | 1975 | CD | PRO | A | 878 | 20.732 | 47.698 | −3.822 | 1.00 | 26.50 | A | C |
| ATOM | 1976 | CA | PRO | A | 878 | 19.010 | 49.166 | −3.002 | 1.00 | 27.36 | A | C |
| ATOM | 1977 | CB | PRO | A | 878 | 18.974 | 47.804 | −2.311 | 1.00 | 26.99 | A | C |
| ATOM | 1978 | CG | PRO | A | 878 | 19.585 | 46.896 | −3.281 | 1.00 | 28.13 | A | c |
| ATOM | 1979 | C | PRO | A | 878 | 19.320 | 50.282 | −2.010 | 1.00 | 25.74 | A | C |
| ATOM | 1980 | O | PRO | A | 878 | 20.478 | 50.509 | −1.673 | 1.00 | 25.44 | A | O |
| ATOM | 1981 | N | ALA | A | 879 | 18.279 | 50.945 | −1.524 | 1.00 | 26.30 | A | N |
| ATOM | 1982 | CA | ALA | A | 879 | 18.445 | 52.056 | −0.592 | 1.00 | 27.29 | A | C |
| ATOM | 1983 | CB | ALA | A | 879 | 17.075 | 52.565 | −0.142 | 1.00 | 25.88 | A | C |
| ATOM | 1984 | C | ALA | A | 879 | 19.307 | 51.742 | 0.624 | 1.00 | 26.75 | A | C |
| ATOM | 1985 | O | ALA | A | 879 | 20.092 | 52.583 | 1.051 | 1.00 | 29.14 | A | O |
| ATOM | 1986 | N | PHE | A | 880 | 19.202 | 50.532 | 1.165 | 1.00 | 25.72 | A | N |
| ATOM | 1987 | CA | PHE | A | 880 | 19.972 | 50.213 | 2.362 | 1.00 | 26.31 | A | C |
| ATOM | 1988 | CB | PHE | A | 880 | 19.047 | 49.627 | 3.428 | 1.00 | 28.37 | A | C |
| ATOM | 1989 | CG | PHE | A | 880 | 17.899 | 50.519 | 3.771 | 1.00 | 29.95 | A | C |
| ATOM | 1990 | CD1 | PHE | A | 880 | 16.678 | 50.384 | 3.123 | 1.00 | 32.51 | A | C |
| ATOM | 1991 | CD2 | PHE | A | 880 | 18.050 | 51.532 | 4.715 | 1.00 | 31.03 | A | C |
| ATOM | 1992 | CE1 | PHE | A | 880 | 15.621 | 51.243 | 3.406 | 1.00 | 33.31 | A | C |
| ATOM | 1993 | CE2 | PHE | A | 880 | 16.998 | 52.399 | 5.006 | 1.00 | 32.39 | A | C |
| ATOM | 1994 | CZ | PHE | A | 880 | 15.784 | 52.257 | 4.353 | 1.00 | 33.31 | A | C |
| ATOM | 1995 | C | PHE | A | 880 | 21.187 | 49.325 | 2.202 | 1.00 | 26.89 | A | C |
| ATOM | 1996 | O | PHE | A | 880 | 21.775 | 48.882 | 3.191 | 1.00 | 26.72 | A | O |
| ATOM | 1997 | N | ALA | A | 881 | 21.582 | 49.078 | 0.962 | 1.00 | 27.32 | A | N |
| ATOM | 1998 | CA | ALA | A | 881 | 22.736 | 48.246 | 0.703 | 1.00 | 27.94 | A | C |
| ATOM | 1999 | CB | ALA | A | 881 | 22.546 | 47.485 | −0.616 | 1.00 | 29.37 | A | C |
| ATOM | 2000 | C | ALA | A | 881 | 24.028 | 49.048 | 0.642 | 1.00 | 30.57 | A | C |
| ATOM | 2001 | O | ALA | A | 881 | 24.118 | 50.052 | −0.066 | 1.00 | 29.11 | A | O |
| ATOM | 2002 | N | PRO | A | 882 | 25.037 | 48.638 | 1.423 | 1.00 | 32.05 | A | N |
| ATOM | 2003 | CD | PRO | A | 882 | 24.988 | 47.755 | 2.601 | 1.00 | 33.91 | A | C |
| ATOM | 2004 | CA | PRO | A | 882 | 26.304 | 49.367 | 1.372 | 1.00 | 34.17 | A | C |
| ATOM | 2005 | CB | PRO | A | 882 | 27.101 | 48.748 | 2.517 | 1.00 | 34.87 | A | C |
| ATOM | 2006 | CG | PRO | A | 882 | 26.037 | 48.372 | 3.497 | 1.00 | 34.72 | A | C |
| ATOM | 2007 | C | PRO | A | 882 | 26.880 | 49.005 | 0.005 | 1.00 | 34.84 | A | C |
| ATOM | 2008 | O | PRO | A | 882 | 26.557 | 47.946 | −0.537 | 1.00 | 32.74 | A | O |
| ATOM | 2009 | N | LYS | A | 883 | 27.712 | 49.873 | −0.560 | 1.00 | 35.18 | A | N |
| ATOM | 2010 | CA | LYS | A | 883 | 28.305 | 49.615 | −1.870 | 1.00 | 36.75 | A | C |
| ATOM | 2011 | CB | LYS | A | 883 | 29.385 | 50.654 | −2.167 | 1.00 | 39.56 | A | C |
| ATOM | 2012 | CG | LYS | A | 883 | 30.149 | 50.408 | −3.463 | 1.00 | 41.70 | A | C |
| ATOM | 2013 | CD | LYS | A | 883 | 31.300 | 51.386 | −3.605 | 1.00 | 46.30 | A | C |
| ATOM | 2014 | CE | LYS | A | 883 | 32.198 | 51.030 | −4.785 | 1.00 | 47.44 | A | C |
| ATOM | 2015 | NZ | LYS | A | 883 | 33.382 | 51.939 | −4.867 | 1.00 | 48.85 | A | N |
| ATOM | 2016 | C | LYS | A | 883 | 28.908 | 48.212 | −2.00 | 41.00 | 36.73 | A | C |
| ATOM | 2017 | O | LYS | A | 883 | 28.779 | 47.571 | −3.047 | 1.00 | 37.14 | A | O |
| ATOM | 2018 | N | ASN | A | 884 | 29.565 | 47.736 | −0.951 | 1.00 | 35.03 | A | N |
| ATOM | 2019 | CA | ASN | A | 884 | 30.192 | 46.418 | −0.987 | 1.00 | 34.74 | A | C |
| ATOM | 2020 | CB | ASN | A | 884 | 31.053 | 46.224 | 0.259 | 1.00 | 37.97 | A | C |
| ATOM | 2021 | CG | ASN | A | 884 | 32.318 | 47.048 | 0.216 | 1.00 | 41.02 | A | C |
| ATOM | 2022 | OD1 | ASN | A | 884 | 32.383 | 48.070 | −0.472 | 1.00 | 42.58 | A | O |
| ATOM | 2023 | ND2 | ASN | A | 884 | 33.332 | 46.617 | 0.958 | 1.00 | 42.68 | A | N |
| ATOM | 2024 | C | ASN | A | 884 | 29.188 | 45.284 | −1.109 | 1.00 | 32.58 | A | C |
| ATOM | 2025 | O | ASN | A | 884 | 29.474 | 44.258 | −1.722 | 1.00 | 32.18 | A | O |
| ATOM | 2026 | N | ILE | A | 885 | 28.018 | 45.469 | −0.512 | 1.00 | 31.95 | A | N |
| ATOM | 2027 | CA | ILE | A | 885 | 26.972 | 44.461 | −0.578 | 1.00 | 30.39 | A | C |
| ATOM | 2028 | CB | ILE | A | 885 | 25.859 | 44.745 | 0.459 | 1.00 | 30.06 | A | C |
| ATOM | 2029 | CC2 | ILE | A | 885 | 24.731 | 43.747 | 0.312 | 1.00 | 30.92 | A | C |
| ATOM | 2030 | CG1 | ILE | A | 885 | 26.445 | 44.705 | 1.871 | 1.00 | 32.44 | A | C |
| ATOM | 2031 | CD1 | ILE | A | 885 | 27.153 | 43.414 | 2.215 | 1.00 | 34.23 | A | C |
| ATOM | 2032 | C | ILE | A | 885 | 26.396 | 44.493 | −1.996 | 1.00 | 28.80 | A | C |
| ATOM | 2033 | O | ILE | A | 885 | 26.090 | 43.451 | −2.572 | 1.00 | 27.47 | A | O |
| ATOM | 2034 | N | TYR | A | 886 | 26.256 | 45.689 | −2.561 | 1.00 | 27.42 | A | N |
| ATOM | 2035 | CA | TYR | A | 886 | 25.734 | 45.805 | −3.918 | 1.00 | 27.23 | A | C |
| ATOM | 2036 | CB | TYR | A | 886 | 25.514 | 47.276 | −4.292 | 1.00 | 27.39 | A | C |
| ATOM | 2037 | CG | TYR | A | 886 | 24.935 | 47.473 | −5.675 | 1.00 | 27.39 | A | C |
| ATOM | 2038. | CD1 | TYR | A | 886 | 23.774 | 46.808 | −6.066 | 1.00 | 27.64 | A | C |
| ATOM | 2039 | CE1 | TYR | A | 886 | 23.239 | 46.977 | −7.342 | 1.00 | 28.02 | A | C |
| ATOM | 2040 | CD2 | TYR | A | 886 | 25.552 | 48.321 | −6.601 | 1.00 | 28.67 | A | C |
| ATOM | 2041 | CE2 | TYR | A | 886 | 25.025 | 48.497 | −7.885 | 1.00 | 28.90 | A | C |
| ATOM | 2042 | CZ | TYR | A | 886 | 23.870 | 47.822 | −8.247 | 1.00 | 28.99 | A | C |
| ATOM | 2043 | OH | TYR | A | 886 | 23.345 | 47.974 | −9.510 | 1.00 | 28.49 | A | O |
| ATOM | 2044 | C | TYR | A | 886 | 26.737 | 45.153 | −4.873 | 1.00 | 27.22 | A | C |
| ATOM | 2045 | O | TYR | A | 886 | 26.356 | 44.594 | −5.899 | 1.00 | 27.84 | A | O |
| ATOM | 2046 | N | SER | A | 887 | 28.021 | 45.218 | −4.524 | 1.00 | 27.56 | A | N |
| ATOM | 2047 | CA | SER | A | 887 | 29.051 | 44.600 | −5.353 | 1.00 | 28.78 | A | C |
| ATOM | 2048 | CB | SER | A | 887 | 30.446 | 44.897 | −4.799 | 1.00 | 31.23 | A | C |
| ATOM | 2049 | OG | SER | A | 887 | 30.700 | 46.291 | −4.802 | 1.00 | 35.41 | A | O |
| ATOM | 2050 | C | SER | A | 887 | 28.843 | 43.088 | −5.417 | 1.00 | 27.15 | A | C |
| ATOM | 2051 | O | SER | A | 887 | 29.180 | 42.461 | −6.411 | 1.00 | 26.08 | A | O |
| ATOM | 2052 | N | ILE | A | 888 | 28.288 | 42.505 | −4.355 | 1.00 | 26.96 | A | N |
| ATOM | 2053 | CA | ILE | A | 888 | 28.035 | 41.068 | −4.340 | 1.00 | 26.66 | A | C |
| ATOM | 2054 | CB | ILE | A | 888 | 27.623 | 40.551 | −2.940 | 1.00 | 26.97 | A | C |
| ATOM | 2055 | CG2 | ILE | A | 888 | 27.329 | 39.049 | −3.014 | 1.00 | 25.87 | A | C |
| ATOM | 2056 | CG1 | ILE | A | 888 | 28.744 | 40.812 | −1.932 | 1.00 | 29.82 | A | C |
| ATOM | 2057 | CD1 | ILEA | 888 | 28.368 | 40.465 | −0.507 | 1.00 | 29.33 | A | C | |
| ATOM | 2058 | C | ILE | A | 888 | 26.913 | 40.763 | −5.314 | 1.00 | 24.54 | A | C |
| ATOM | 2059 | O | ILE | A | 888 | 26.991 | 39.801 | −6.067 | 1.00 | 24.10 | A | O |
| ATOM | 2060 | N | MET | A | 889 | 25.872 | 41.596 | −5.295 | 1.00 | 24.37 | A | N |
| ATOM | 2061 | CA | MET | A | 889 | 24.733 | 41.426 | −6.187. | 1.00.24.31 | A.C | | |
| ATOM | 2062 | CB | MET | A | 889 | 23.721 | 42.558 | 5.981 | 1.00 | 24.18 | A | C |
| ATOM | 2063 | CG | MET | A | 889 | 23.021 | 42.541 | −4.614 | 1.00 | 24.24 | A | C |
| ATOM | 2064 | SD | MET | A | 889 | 21.876 | 43.943 | −4.442 | 1.00 | 24.46 | A | S |
| ATOM | 2065 | CE | MET | A | 889 | 22.192 | 44.419 | −2.726 | 1.00 | 24.20 | A | C |
| ATOM | 2066 | C | MET | A | 889 | 25.193 | 41.421 | −7.640 | 1.00 | 25.77 | A | C |
| ATOM | 2067 | O | MET | A | 889 | 24.802 | 40.550 | −8.429 | 1.00 | 23.93 | A | O |
| ATOM | 2068 | N | GLN | A | 890 | 26.035 | 42.394 | −7.978 | 1.00 | 26.71 | A | N |
| ATOM | 2069 | CA | GLN | A | 890 | 26.561 | 42.525 | −9.332 | 1.00 | 28.97 | A | C |
| ATOM | 2070 | CB | GLN | A | 890 | 27.442 | 43.776 | −9.429 | 1.00 | 29.48 | A | C |
| ATOM | 2071 | CG | GLN | A | 890 | 26.668 | 45.076 | −9.219 | 1.00 | 30.61 | A | C |
| ATOM | 2072 | CD | GLN | A | 890 | 27.546 | 46.309 | −9.285 | 1.00 | 32.25 | A | C |
| ATOM | 2073 | OE1 | GLN | A | 890 | 28.447 | 46.488 | −8.468 | 1.00 | 33.10 | A | O |
| ATOM | 2074 | NE2 | GLN | A | 890 | 27.279 | 47.173 | −10.259 | 1.00 | 32.57 | A | N |
| ATOM | 2075 | C | GLN | A | 890 | 27.350 | 41.287 | −9.739 | 1.00 | 27.86 | A | C |
| ATOM | 2076 | O | GLN | A | 890 | 27.247 | 40.826 | −10.878 | 1.00 | 30.13 | A | O |
| ATOM | 2077 | N | ALA | A | 891 | 28.133 | 40.755 | −8.806 | 1.00 | 27.34 | A | N |
| ATOM | 2078 | CA | ALA | A | 891 | 28.934 | 39.560 | −9.058 | 1.00 | 27.20 | A | C |
| ATOM | 2079 | CB | ALA | A | 891 | 29.835 | 39.270 | −7.858 | 1.00 | 27.77 | A | C |
| ATOM | 2080 | C | ALA | A | 891 | 28.011 | 38.378 | −9.331 | 1.00 | 26.78 | A | C |
| ATOM | 2081 | O | ALA | A | 891 | 28.248 | 37.598 | −10.252 | 1.00 | 25.81 | A | O |
| ATOM | 2082 | N | CYS | A | 892 | 26.954 | 38.255 | −8.528 | 1.00 | 25.33 | A | N |
| ATOM | 2083 | CA | CYS | A | 892 | 25.977 | 37.183 | −8.694 | 1.00 | 23.44 | A | C |
| ATOM | 2084 | CB | CYS | A | 892 | 24.912 | 37.234 | −7.586 | 1.00 | 21.37 | A | C |
| ATOM | 2085 | SG | CYS | A | 892 | 25.501 | 36.833 | −5.923 | 1.00 | 25.09 | A | S |
| ATOM | 2086 | C | CYS | A | 892 | 25.259 | 37.287 | −10.032 | 1.00 | 22.41 | A | C |
| ATOM | 2087 | O | CYS | A | 892 | 24.802 | 36.279 | −10.576 | 1.00 | 21.96 | A | O |
| ATOM | 2088 | N | TRP | A | 893 | 25.138 | 38.513 | −10.540 | 1.00 | 22.92 | A | N |
| ATOM | 2089 | CA | TRP | A | 893 | 24.445 | 38.748 | −11.802 | 1.00 | 24.15 | A | C |
| ATOM | 2090 | CB | TRP | A | 893 | 23.640 | 40.049 | −11.731 | 1.00 | 23.99 | A | C |
| ATOM | 2091 | CG | TRP | A | 893 | 22.602 | 40.070 | −10.621 | 1.00 | 25.25 | A | C |
| ATOM | 2092 | CD2 | TRP | A | 893 | 22.139 | 41.219 | −9.905 | 1.00 | 24.67 | A | C |
| ATOM | 2093 | CE2 | TRP | A | 893 | 21.168 | 40.770 | −8.971 | 1.00 | 25.85 | A | C |
| ATOM | 2094 | CE3 | TRP | A | 893 | 22.449 | 42.588 | −9.957 | 1.00 | 24.95 | A | C |
| ATOM | 2095 | CD1 | TRP | A | 893 | 21.911 | 38.997 | −10.113 | 1.00 | 26.64 | A | C |
| ATOM | 2096 | NE1 | TRP | A | 893 | 21.050 | 39.412 | −9.118 | 1.00 | 24.18 | A | N |
| ATOM | 2097 | CZ2 | TRP | A | 893 | 20.505 | 41.644 | −8.097 | 1.00 | 23.40 | A | C |
| ATOM | 2098 | CZ3 | TRP | A | 893 | 21.793 | 43.457 | −9.085 | 1.00 | 22.62 | A | C |
| ATOM | 2099 | CH2 | TRP | A | 893 | 20.827 | 42.977 | −8.166 | 1.00 | 21.93 | A | C |
| ATOM | 2100 | C | TRP | A | 893 | 25.344 | 38.773 | −13.043 | 1.00 | 27.09 | A | C |
| ATOM | 2101 | O | TRP | A | 893 | 24.948 | 39.287 | −14.092 | 1.00 | 27.52 | A | O |
| ATOM | 2102 | N | ALA | A | 894 | 26.553 | 38.238 | −12.926 | 1.00 | 30.21 | A | N |
| ATOM | 2103 | CA | ALA | A | 894 | 27.443 | 38.182 | −14.085 | 1.00 | 31.38 | A | C |
| ATOM | 2104 | CB | ALA | A | 894 | 28.788 | 37.593 | −13.686 | 1.00 | 32.39 | A | C |
| ATOM | 2105 | C | ALA | A | 894 | 26.736 | 37.267 | −15.084 | 1.00 | 31.53 | A | C |
| ATOM | 2106 | O | ALA | A | 894 | 26.229 | 36.215 | −14.705 | 1.00 | 30.02 | A | O |
| ATOM | 2107 | N | LEU | A | 895 | 26.680 | 37.670 | −16.351 | 1.00 | 32.91 | A | N |
| ATOM | 2108 | CA | LEU | A | 895 | 26.011 | 36.866 | −17.367 | 1.00 | 33.93 | A | C |
| ATOM | 2109 | CB | LEU | A | 895 | 25.980 | 37.612 | −18.707 | 1.00 | 34.40 | A | C |
| ATOM | 2110 | CG | LEU | A | 895 | 25.046 | 38.827 | −18.789 | 1.00 | 37.38 | A | C |
| ATOM | 2111 | CD1 | LEU | A | 895 | 25.127 | 39.442 | −20.180 | 1.00 | 38.41 | A | C |
| ATOM | 2112 | CD2 | LEU | A | 895 | 23.619 | 38.405 | −18.487 | 1.00 | 36.13 | A | C |
| ATOM | 2113 | C | LEU | A | 895 | 26.671 | 35.503 | −17.544 | 1.00 | 34.31 | A | C |
| ATOM | 2114 | O | LEU | A | 895 | 25.994 | 34.497 | −17.740 | 1.00 | 34.78 | A | O |
| ATOM | 2115 | N | GLU | A | 896 | 27.995 | 35.478 | −17.472 | 1.00 | 37.15 | A | N |
| ATOM | 2116 | CA | GLU | A | 896 | 28.745 | 34.238 | −17.607 | 1.00 | 39.52 | A | C |
| ATOM | 2117 | CB | GLU | A | 896 | 30.185 | 34.564 | −18.025 | 1.00 | 42.43 | A | C |
| ATOM | 2118 | CG | GLU | A | 896 | 31.080 | 33.366 | −18.278 | 1.00 | 48.49 | A | C |
| ATOM | 2119 | CD | GLU | A | 896 | 32.398 | 33.763 | −18.927 | 1.00 | 52.37 | A | C |
| ATOM | 2120 | OE1 | GLU | A | 896 | 33.134 | 34.588 | −18.336 | 1.00 | 53.43 | A | O |
| ATOM | 2121 | OE2 | GLU | A | 896 | 32.695 | 33.254 | −20.033 | 1.00 | 54.83 | A | O |
| ATOM | 2122 | C | GLU | A | 896 | 28.720 | 33.520 | −16.251 | 1.00 | 37.75 | A | C |
| ATOM | 2123 | O | GLU | A | 896 | 29.301 | 33.998 | −15.280 | 1.00 | 37.75 | A | O |
| ATOM | 2124 | N | PRO | A | 897 | 28.031 | 32.370 | −16.170 | 1.00 | 37.87 | A | N |
| ATOM | 2125 | CD | PRO | A | 897 | 27.209 | 31.770 | −17.237 | 1.00 | 37.58 | A | C |
| ATOM | 2126 | CA | PRO | A | 897 | 27.922 | 31.581 | −14.937 | 1.00 | 37.70 | A | C |
| ATOM | 2127 | CB | PRO | A | 897 | 27.250 | 30.301 | −15.419 | 1.00 | 37.56 | A | C |
| ATOM | 2128 | CG | PRO | A | 897 | 26.322 | 30.816 | −16.465 | 1.00 | 37.35 | A | C |
| ATOM | 2129 | C | PRO | A | 897 | 29.226 | 31.312 | −14.189 | 1.00 | 38.10 | A | C |
| ATOM | 2130 | O | PRO | A | 897 | 29.242 | 31.281 | −12.955 | 1.00 | 36.69 | A | O |
| ATOM | 2131 | N | THR | A | 898 | 30.320 | 31.124 | −14.923 | 1.00 | 38.36 | A | N |
| ATOM | 2132 | CA | THR | A | 898 | 31.608 | 30.849 | −14.288 | 1.00 | 37.76 | A | C |
| ATOM | 2133 | CB | THR | A | 898 | 32.593 | 30.172 | −15.268 | 1.00 | 38.94 | A | C |
| ATOM | 2134 | OG1 | THR | A | 898 | 32.951 | 31.093 | −16.304 | 1.00 | 40.73 | A | O |
| ATOM | 2135 | CG2 | THR | A | 898 | 31.957 | 28.941 | −15.889 | 1.00 | 39.66 | A | C |
| ATOM | 2136 | C | THR | A | 898 | 32.265 | 32.108 | −13.746 | 1.00 | 37.96 | A | C |
| ATOM | 2137 | O | THR | A | 898 | 33.294 | 32.038 | −13.072 | 1.00 | 38.29 | A | O |
| ATOM | 2138 | N | HIS | A | 899 | 31.681 | 33.264 | −14.043 | 1.00 | 37.07 | A | N |
| ATOM | 2139 | CA | HIS | A | 899 | 32.233 | 34.519 | −13.562 | 1.00 | 37.73 | A | C |
| ATOM | 2140 | CB | HIS | A | 899 | 32.081 | 35.605 | −14.623 | 1.00 | 42.21 | A | C |
| ATOM | 2141 | CG | HIS | A | 899 | 33.278 | 36.494 | −14.742 | 1.00 | 48.88 | A | C |
| ATOM | 2142 | CD2 | HIS | A | 899 | 34.151 | 36.682 | −15.761 | 1.00 | 50.32 | A | C |
| ATOM | 2143 | ND1 | HIS | A | 899 | 33.710 | 37.306 | −13.713 | 1.00 | 90.96 | A | N |
| ATOM | 2144 | CE1 | HIS | A | 899 | 34.796 | 37.955 | −14.094 | 1.00 | 51.86 | A | C |
| ATOM | 2145 | NE2 | HIS | A | 899 | 35.085 | 37.593 | −15.333 | 1.00 | 53.20 | A | N |
| ATOM | 2146 | C | HIS | A | 899 | 31.543 | 34.946 | −12.268 | 1.00 | 35.74 | A | C |
| ATOM | 2147 | O | HIS | A | 899 | 31.897 | 35.964 | −11.665 | 1.00 | 35.35 | A | C |
| ATOM | 2148 | N | ARG | A | 900 | 30.554 | 34.160 | −11.854 | 1.00 | 33.68 | A | N |
| ATOM | 2149 | CA | ARG | A | 900 | 29.819 | 34.422 | −10.618 | 1.00 | 32.79 | A | C |
| ATOM | 2150 | CB | ARG | A | 900 | 28.419 | 33.797 | −10.673 | 1.00 | 31.57 | A | C |
| ATOM | 2151 | CG | ARG | A | 900 | 27.532 | 34.351 | −11.759 | 1.00 | 31.01 | A | C |
| ATOM | 2152 | CD | ARG | A | 900 | 26.215 | 33.604 | −11.853 | 1.00 | 28.64 | A | C |
| ATOM | 2153 | NE | ARG | A | 900 | 25.516 | 33.987 | −13.071 | 1.00 | 29.23 | A | N |
| ATOM | 2154 | CZ | ARG | A | 900 | 24.644 | 33.223 | −13.714 | 1.00 | 27.03 | A | C |
| ATOM | 2155 | NH1 | ARG | A | 900 | 24.335 | 32.012 | −13.252 | 1.00 | 28.97 | A | N |
| ATOM | 2156 | NH2 | ARG | A | 900 | 24.120 | 33.652 | −14.853 | 1.00 | 29.12 | A | N |
| ATOM | 2157 | C | ARG | A | 900 | 30.589 | 33.778 | −9.482 | 1.00 | 32.53 | A | C |
| ATOM | 2158 | O | ARG | A | 900 | 31.235 | 32.747 | −9.665 | 1.00 | 32.70 | A | O |
| ATOM | 2159 | N | PRO | A | 901 | 30.530 | 34.371 | −8.287 | 1.00 | 31.70 | A | N |
| ATOM | 2160 | CD | PRO | A | 901 | 29.816 | 35.591 | −7.868 | 1.00 | 31.21 | A | C |
| ATOM | 2161 | CA | PRO | A | 901 | 31.257 | 33.773 | −7.169 | 1.00 | 31.11 | A | C |
| ATOM | 2162 | CB | PRO | A | 901 | 31.205 | 34.866 | −6.112 | 1.00 | 31.76 | A | C |
| ATOM | 2163 | CG | PRO | A | 901 | 29.852 | 35.482 | −6.358 | 1.00 | 32.84 | A | C |
| ATOM | 2164 | C | PRO | A | 901 | 30.556 | 32.499 | −6.708 | 1.00 | 32.06 | A | C |
| ATOM | 2165 | O | PRO | A | 901 | 29.437 | 32.203 | −7.138 | 1.00 | 29.97 | A | O |
| ATOM | 2166 | N | THR | A | 902 | 31.221 | 31.740 | −5.844 | 1.00 | 31.96 | A | N |
| ATOM | 2167 | CA | THR | A | 902 | 30.616 | 30.530 | −5.302 | 1.00 | 29.99 | A | C |
| ATOM | 2168 | CB | THR | A | 902 | 31.675 | 29.455 | −5.000 | 1.00 | 28.30 | A | C |
| ATOM | 2169 | OG1 | THR | A | 902 | 32.517 | 29.905 | −3.938 | 1.00 | 28.94 | A | O |
| ATOM | 2170 | CG2 | THR | A | 902 | 32.524 | 29.184 | −6.236 | 1.00 | 29.96 | A | C |
| ATOM | 2171 | C | THR | A | 902 | 29.976 | 30.973 | −3.991 | 1.00 | 28.78 | A | C |
| ATOM | 2172 | O | THR | A | 902 | 30.268 | 32.065 | −3.502 | 1.00 | 28.62 | A | O |
| ATOM | 2173 | N | PHE | A | 903 | 29.100 | 30.154 | 3.421 | 1.00 | 28.57 | A | N |
| ATOM | 2174 | CA | PHE | A | 903 | 28.478 | 30.536 | −2.157 | 1.00 | 29.13 | A | C |
| ATOM | 2175 | CB | PHE | A | 903 | 27.343 | 29.570 | −1.787 | 1.00 | 28.81 | A | C |
| ATOM | 2176 | CG | PHE | A | 903 | 26.064 | 29.835 | −2.532 | 1.00 | 27.49 | A | C |
| ATOM | 2177 | CD1 | PHE | A | 903 | 25.381 | 31.034 | −2.348 | 1.00 | 25.90 | A | C |
| ATOM | 2178 | CD2 | PHE | A | 903 | 25.558 | 28.906 | −3.435 | 1.00 | 26.86 | A | C |
| ATOM | 2179 | CE1 | PHE | A | 903 | 24.215 | 31.308 | −3.051 | 1.00 | 22.70 | A | C |
| ATOM | 2180 | CE2 | PHE | A | 903 | 24.385 | 29.169 | −4.149 | 1.00 | 25.78 | A | C |
| ATOM | 2181 | CZ | PHE | A | 903 | 23.713 | 30.372 | −3.957 | 1.00 | 23.53 | A | C |
| ATOM | 2182 | C | PHE | A | 903 | 29.516 | 30.586 | −1.045 | 1.00 | 30.49 | A | C |
| ATOM | 2183 | O | PHE | A | 903 | 29.411 | 31.403 | −0.128 | 1.00 | 29.48 | A | O |
| ATOM | 2184 | N | GLN | A | 904 | 30.534 | 29.732 | −1.134 | 1.00 | 32.67 | A | N |
| ATOM | 2185 | CA | GLN | A | 904 | 31.582 | 29.731 | −0.119 | 1.00 | 34.69 | A | C |
| ATOM | 2186 | CB | GLN | A | 904 | 32.546 | 28.563 | −0.342 | 1.00 | 38.18 | A | C |
| ATOM | 2187 | CG | GLN | A | 904 | 33.580 | 28.423 | 0.761 | 1.00 | 43.01 | A | C |
| ATOM | 2188 | CD | GLN | A | 904 | 32.967 | 28.567 | 2.142 | 1.00 | 45.54 | A | C |
| ATOM | 2189 | OE1 | GLN | A | 904 | 32.147 | 27.750 | 2.562 | 1.00 | 47.82 | A | O |
| ATOM | 2190 | NE2 | GLN | A | 904 | 33.354 | 29.622 | 2.852 | 1.00 | 47.75 | A | N |
| ATOM | 2191 | C | GLN | A | 904 | 32.345 | 31.061 | −0.140 | 1.00 | 34.49 | A | C |
| ATOM | 2192 | O | GLN | A | 904 | 32.704 | 31.606 | 0.904 | 1.00 | 32.29 | A | O |
| ATOM | 2193 | N | GLN | A | 905 | 32.585 | 31.585 | −1.337 | 1.00 | 35.55 | A | N |
| ATOM | 2194 | CA | GLN | A | 905 | 33.277 | 32.855 | −1.471 | 1.00 | 35.22 | A | C |
| ATOM | 2195 | CB | GLN | A | 905 | 33.669 | 33.086 | −2.930 | 1.00 | 35.06.A | C | |
| ATOM | 2196 | CG | GLN | A | 905 | 34.894 | 32.287 | −3.331 | 1.00 | 38.42 | A | C |
| ATOM | 2197 | CD | GLN | A | 905 | 35.079 | 32.173 | −4.827 | 1.00 | 38.16 | A | C |
| ATOM | 2198 | OE1 | GLN | A | 905 | 36.085 | 31.636 | −5.297 | 1.00 | 43.96 | A | O |
| ATOM | 2199 | NE2 | GLN | A | 905 | 34.113 | 32.663 | −5.586 | 1.00 | 36.11 | A | N |
| ATOM | 2200 | C | GLN | A | 905 | 32.397 | 33.991 | −0.962 | 1.00 | 35.01 | A | C |
| ATOM | 2201 | O | GLN | A | 905 | 32.887 | 34.948 | −0.363 | 1.00 | 34.50 | A | O |
| ATOM | 2202 | N | ILE | A | 906 | 31.091 | 33.877 | −1.186 | 1.00 | 34.36 | A | N |
| ATOM | 2203 | CA | ILE | A | 906 | 30.168 | 34.901 | −0.721 | 1.00 | 33.30 | A | C |
| ATOM | 2204 | CB | ILE | A | 906 | 28.744 | 34.634 | −1.257 | 1.00 | 30.88 | A | C |
| ATOM | 2205 | CG2 | ILE | A | 906 | 27.713 | 35.434 | −0.466 | 1.00 | 30.71 | A | C |
| ATOM | 2206 | CG1 | ILE | A | 906 | 28.697 | 34.989 | −2.748 | 1.00 | 31.17 | A | C |
| ATOM | 2207 | CD1 | ILE | A | 906 | 27.396 | 34.636 | −3.434 | 1.00 | 28.64 | A | C |
| ATOM | 2208 | C | ILE | A | 906 | 30.181 | 34.941 | 0.808 | 1.00 | 34.77 | A | C |
| ATOM | 2209 | O | ILE | A | 906 | 30.255 | 36.018 | 1.406 | 1.00 | 31.91 | A | O |
| ATOM | 2210 | N | CYS | A | 907 | 30.129 | 33.768 | 1.433 | 1.00 | 36.66 | A | N |
| ATOM | 2211 | CA | CYS | A | 907 | 30.158 | 33.674 | 2.894 | 1.00 | 40.20 | A | C |
| ATOM | 2212 | GB | CYS | A | 907 | 30.198 | 32.213 | 3.338 1.00 | 39.93 | A | C | |
| ATOM | 2213 | SG | CYS | A | 907 | 28.611 | 31.428 | 3.449 | 1.00 | 44.12 | A | S |
| ATOM | 2214 | C | CYS | A | 907 | 31.368 | 34.374 | 3.490 | 1.00 | 41.65 | A | C |
| ATOM | 2215 | O | CYS | A | 907 | 31.245 | 35.172 | 4.420 | 1.00 | 42.40 | A | O |
| ATOM | 2216 | N | SER | A | 908 | 32.538 | 34.053 | 2.949 | 1.00 | 43.49 | A | N |
| ATOM | 2217 | CA | SER | A | 908 | 33.794 | 34.619 | 3.417 | 1.00 | 45.17 | A | C |
| ATOM | 2218 | GB | SER | A | 908 | 34.953 | 34.051 | 2.599 | 1.00 | 46.62 | A | C |
| ATOM | 2219 | OG | SER | A | 908 | 36.199 | 34.452 | 3.139 | 1.00 | 50.76 | A | O |
| ATOM | 2220 | C | SER | A | 908 | 33.801 | 36.137 | 3.324 | 1.00 | 45.72 | A | C |
| ATOM | 2221 | O | SER | A | 908 | 34.236 | 36.826 | 4.251 | 1.00 | 45.92 | A | O |
| ATOM | 2222 | N | PHE | A | 909 | 33.319 | 36.655 | 2.199 | 1.00 | 45.06 | A | N |
| ATOM | 2223 | CA | PHE | A | 909 | 33.274 | 38.093 | 1.977 | 1.00 | 44.08 | A | C |
| ATOM | 2224 | GB | PHE | A | 909 | 32.807 | 38.384 | 0.553 | 1.00 | 42.79 | A | C |
| ATOM | 2225 | CG | PHE | A | 909 | 32.905 | 39.828 | 0.166 | 1.00 | 43.94 | A | C |
| ATOM | 2226 | CD1 | PHE | A | 909 | 34.140 | 40.470 | 0.130 | 1.00 | 42.21 | A | C |
| ATOM | 2227 | CD2 | PHE | A | 909 | 31.768 | 40.543 | −0.193 | 1.00 | 41.49 | A | C |
| ATOM | 2228 | CE1 | PHE | A | 909 | 34.239 | 41.799 | −0.259 | 1.00 | 42.41 | A | C |
| ATOM | 2229 | CE2 | PHE | A | 909 | 31.859 | 41.874 | −0.585 | 1.00 | 42.63 | A | C |
| ATOM | 2230 | CZ | PHE | A | 909 | 33.097 | 42.503 | −0.620 | 1.00 | 42.40 | A | C |
| ATOM | 2231 | C | PHE | A | 909 | 32.333 | 38.745 | 2.983 | 1.00 | 44.82 | A | C |
| ATOM | 2232 | O | PHE | A | 909 | 32.633 | 39.808 | 3.528 | 1.00 | 45.91 | A | O |
| ATOM | 2233 | NLEU | A | 910 | 31.191 | 38.109 | 3.223 | 1.00 | 44.06 | A | N | |
| ATOM | 2234 | CA | LEU | A | 910 | 30.227 | 38.625 | 4.181 | 1.00 | 45.22 | A | C |
| ATOM | 2235 | CB | LEU | A | 910 | 28.911 | 37.852 | 4.089 | 1.00 | 41.44 | A | C |
| ATOM | 2236 | CG | LEU | A | 910 | 28.077 | 38.064 | 2.827 | 1.00 | 39.78 | A | C |
| ATOM | 2237 | CD1 | LEU | A | 910 | 26.881 | 37.122 | 2.853 | 1.00 | 39.13 | A | C |
| ATOM | 2238 | CD2 | LEU | A | 910 | 27.619 | 39.519. 2.748 | 1.00 | 38.00 | A | C | |
| ATOM | 2239 | C | LEU | A | 910 | 30.808 | 38.482 | 5.582 | 1.00 | 47.78 | A | C |
| ATOM | 2240 | O | LEU | A | 910 | 30.569 | 39.318 | 6.457 | 1.00 | 47.63 | A | O |
| ATOM | 2241 | N | GLN | A | 911 | 31.574 | 37.416 | 5.788 | 1.00 | 51.82 | A | N |
| ATOM | 2242 | CA | GLN | A | 911 | 32.193 | 37.161 | 7.084 | 1.00 | 56.05 | A | C |
| ATOM | 2243 | CB | GLN | A | 911 | 32.944 | 35.826. 7.051 | 1.00 | 57.62 | A | C | |
| ATOM | 2244 | CG | GLN | A | 911 | 33.337 | 35.290 | 8.417 | 1.00 | 60.18 | A | C |
| ATOM | 2245 | CD | GLN | A | 911 | 33.861 | 33.862 | 8.345 | 1.00 | 61.94 | A | C |
| ATOM | 2246 | OE1 | GLN | A | 911 | 34.842 | 33.587 | 7.655 | 1.00 | 62.84 | A | O |
| ATOM | 2247 | NE2 | GLN | A | 911 | 33.211 | 32.948 | 9.059 | 1.00 | 62.59 | A | N |
| ATOM | 2248 | C | GLN | A | 911 | 33.153 | 38.314 | 7.381 | 1.00 | 58.14 | A | C |
| ATOM | 2249 | O | GLN | A | 911 | 33.185 | 38.838 | 8.493 | 1.00 | 58.49 | A | O |
| ATOM | 2250 | N | GLU | A | 912 | 33.919 | 38.712 | 6.369 | 1.00 | 60.16 | A | N |
| ATOM | 2251 | CA | GLU | A | 912 | 34.858 | 39.813 | 6.509 | 1.00 | 62.99 | A | C |
| ATOM | 2252 | CB | GLU | A | 912 | 35.750 | 39.916 | 5.273 | 1.00 | 63.37 | A | C |
| ATOM | 2253 | CG | GLU | A | 912 | 36.509 | 38.642 | 4.945 | 1.00 | 64.99 | A | C |
| ATOM | 2254 | CD | GLU | A | 912 | 37.419 | 38.809 | 3.741 | 1.00 | 66.51 | A | C |
| ATOM | 2255 | OE1 | GLU | A | 912 | 36.923 | 39.203 | 2.662 | 1.00 | 66.38 | A | O |
| ATOM | 2256 | OE2 | GLU | A | 912 | 38.632 | 38.545 | 3.867 | 1.00 | 67.43 | A | O |
| ATOM | 2257 | C | GLU | A | 912 | 34.060 | 41.101 | 6.657 | 1.00 | 64.48 | A | C |
| ATOM | 2258 | O | GLU | A | 912 | 34.620 | 42.186 | 6.837 | 1.00 | 64.98 | A | O |
| ATOM | 2259 | N | GLN | A | 913 | 32.742 | 40.954 | 6.577 | 1.00 | 66.11 | A | N |
| ATOM | 2260 | GA | GLN | A | 913 | 31.798 | 42.054 | 6.678 | 1.00 | 67.44 | A | C |
| ATOM | 2261 | CB | GLN | A | 913 | 32.214 | 43.024 | 7.781 | 1.00 | 68.94 | A | C |
| ATOM | 2262 | CG | GLN | A | 913 | 32.210 | 42.381 | 9.160 | 1.00 | 70.90 | A | C |
| ATOM | 2263 | CD | GLN | A | 913 | 32.575 | 43.351 | 10.268 | 1.00 | 72.14 | A | C |
| ATOM | 2264 | OE1 | GLN | A | 913 | 33.665 | 43.920 | 10.271 | 1.00 | 73.21 | A | O |
| ATOM | 2265 | NE2 | GLN | A | 913 | 31.662 | 43.548 | 11.214 | 1.00 | 72.27 | A | N |
| ATOM | 2266 | C | GLN | A | 913 | 31.681 | 42.774 | 5.344 | 1.00 | 67.51 | A | C |
| ATOM | 2267 | O | GLN | A | 913 | 30.582 | 42.920 | 4.806 | 1.00 | 68.02 | A | O |
| ATOM | 2268 | N | ALA | A | 914 | 32.815 | 43.202 | 4.800 | 1.00 | 67.46 | A | N |
| ATOM | 2269 | GA | ALA | A | 914 | 32.840 | 43.906 | 3.520 | 1.00 | 66.59 | A | C |
| ATOM | 2270 | CB | ALA | A | 914 | 32.120 | 43.085 | 2.464 | 1.00 | 66.19 | A | C |
| ATOM | 2271 | C | ALA | A | 914 | 32.194 | 45.284 | 3.656 | 1.00 | 66.26 | A | C |
| ATOM | 2272 | O | ALA | A | 914 | 32.708 | 46.273 | 3.134 | 1.00 | 66.94 | A | O |
| ATOM | 2273 | N | GLN | A | 915 | 31.072 | 45.337 | 4.370 | 1.00 | 66.11 | A | N |
| ATOM | 2274 | CA | GLN | A | 915 | 30.343 | 46.580 | 4.604 | 1.00 | 65.66 | A | C |
| ATOM | 2275 | CB | GLN | A | 915 | 29.936 | 47.211 | 3.273 | 1.00 | 65.97 | A | C |
| ATOM | 2276 | CG | GLN | A | 915 | 30.859 | 48.328 | 2.828 | 1.00 | 66.33 | A | C |
| ATOM | 2277 | CD | GLN | A | 915 | 30.128 | 49.641 | 2.672 | 1.00 | 67.07 | A | C |
| ATOM | 2278 | OE1 | GLN | A | 915 | 29.554 | 50.164 | 3.632 | 1.00 | 67.25 | A | O |
| ATOM | 2279 | NE2 | GLN | A | 915 | 30.135 | 50.181 | 1.458 | 1.00 | 66.90 | A | N |
| ATOM | 2280 | C | GLN | A | 915 | 29.109 | 46.378 | 5.483 | 1.00 | 64.80 | A | C |
| ATOM | 2281 | O | GLN | A | 915 | 28.468 | 45.328 | 5.440 | 1.00 | 65.35 | A | O |
| ATOM | 2282 | N | ALA | A | 916 | 28.782 | 47.393 | 6.280 1.00 | 63.80 | A | N | |
| ATOM | 2283 | CA | ALA | A | 916 | 27.628 | 47.328 | 7.171 | 1.00 | 62.26 | A | C |
| ATOM | 2284 | CB | ALA | A | 916 | 28.094 | 47.384 | 8.638 | 1.00 | 60.15 | A | C |
| ATOM | 2285 | C | ALA | A | 916 | 26.671 | 48.473 | 6.889 | 1.00 | 61.91 | A | C |
| ATOM | 2286 | O | ALA | A | 916 | 25.554 | 48.197 | 6.387 | 1.00 | 61.71 | A | O |
| ATOM | 2287 | OXT | ALA | A | 916 | 27.030 | 4.9.6487.168 | 1.00 | 61.76 | A | O | |
| ATOM | 2288 | C1 | INH | I | 1000 | 4.821 | 29.492 | 6.694 | 1.00 | 31.11 | I | C |
| ATOM | 2289 | C2 | INH | I | 1000 | 4.066 | 30.667 | 7.019 | 1.00 | 33.00 | I | C |
| ATOM | 2290 | C3 | INH | I | 1000 | 4.010 | 31.171 | 8.345 | 1.00 | 32.48 | I | C |
| ATOM | 2291 | C4 | INH | I | 1000 | 4.712 | 30.516 | 9.396 | 1.00 | 31.97 | I | C |
| ATOM | 2292 | C5 | INH | I | 1000 | 5.468 | 29.342 | 9.098 | 1.00 | 31.58 | I | C |
| ATOM | 2293 | C6 | INH | I | 1000 | 5.530 | 28.816 | 7.763 | 1.00 | 29.91 | I | C |
| ATOM | 2294 | C7 | INH | I | 1000 | 6.308 | 28.520 | 3.306 | 1.00 | 29.12 | I | C |
| ATOM | 2295 | C8 | INH | I | 1000 | 6.200 | 29.300 | 4.626 | 1.00 | 30.39 | I | C |
| ATOM | 2296 | N1 | INH | I | 1000 | 4.890 | 29.102 | 5.331 | 1.00 | 32.22 | I | N |
| ATOM | 2297 | C9 | INH | I | 1000 | 3.620 | 28.770 | 4.602 | 1.00 | 31.09 | I | C |
| ATOM | 2298 | C10 | INH | I | 1000 | 3.857 | 28.041 | 3.260 | 1.00 | 30.49 | I | C |
| ATOM | 2299 | C11 | INH | I | 1000 | 5.038 | 28.628 | 2.468 | 1.00 | 30.53 | I | C |
| ATOM | 2300 | C12 | INH | I | 1000 | 5.224 | 27.874 | 1.154 | 1.00 | 27.13 | I | C |
| ATOM | 2301 | C13 | INH | I | 1000 | 4.658 | 31.047 | 10.801 | 1.00 | 34.41 | I | C |
| ATOM | 2302 | O1 | INH | I | 1000 | 3.393 | 30.696 | 11.364 | 1.00 | 38.64 | I | O |
| ATOM | 2303 | C14 | INH | I | 1000 | 7.069 | 26.794 | 8.131 | 1.00 | 28.71 | I | C |
| ATOM | 2304 | N2 | INH | I | 1000 | 6.197 | 27.613 | 7.430 | 1.00 | 28.05 | I | N |
| ATOM | 2305 | 02 | INH | I | 1000 | 7.478 | 26.985 | 9.280 | 1.00 | 30.81 | I | O |
| ATOM | 2306 | C16 | INH | I | 1000 | 7.448 | 25.614 | 7.357 | 1.00 | 26.57 | I | C |
| ATOM | 2307 | C16 | INH | I | 1000 | 8.285 | 24.552 | 7.649 | 1.00 | 26.78 | I | C |
| ATOM | 2308 | C17 | INH | I | 1000 | 8.266 | 23.688 | 6.517 | 1.00 | 28.27 | I | C |
| ATOM | 2309 | C18 | INH | I | 1000 | 7.418 | 24.279 | 5.603 | 1.00 | 26.96 | I | C |
| ATOM | 2310 | O3 | INH | I | 1000 | 6.918 | 25.440 | 6.094 | 1.00 | 27.67 | I | O |
| ATOM | 2311 | C19 | INH | I | 1000 | 7.010 | 23.900 | 4.300 | 1.00 | 27.52 | I | C |
| ATOM | 2312 | N3 | INH | I | 1000 | 6.683 | 23.601 | 3.228 | 1.00 | 28.92 | I | N |
| ATOM | 2313 | O | HOH | W | 1 | 10.164 | 40.940 | −3.146 | 1.00 | 21.08 | W | O |
| ATOM | 2314 | O | HOH | W | 2 | 7.936 | 37.125 | −3.614 | 1.00 | 22.66 | W | O |
| ATOM | 2315 | O | HOH | W | 3 | 13.953 | 27.823 | 0.701 | 1.00 | 25.69 | W | O |
| ATOM | 2316 | O | HOH | W | 4 | 16.609 | 38.256 | −12.304 | 1.00 | 24.24 | W | O |
| ATOM | 2317 | O | HOH | W | 5 | 15.463 | 37.003 | −14.494 | 1.00 | 28.69 | W | O |
| ATOM | 2318 | O | HOH | W | 6 | 12.958 | 32.094 | −7.055 | 1.00 | 23.76 | W | O |
| ATOM | 2319 | O | HOH | W | 7 | 8.733 | 36.704 | −11.363 | 1.00 | 25.45 | W | O |
| ATOM | 2320 | O | HOH | W | 8 | 10.417 | 36.756 | −2.552 | 1.00 | 23.52 | W | O |
| ATOM | 2321 | O | HOH | W | 9 | 17.184 | 29.603 | −7.251 | 1.00 | 25.10 | W | O |
| ATOM | 2322 | O | HOH | W | 10 | 21.459 | 40.971 | −16.856 | 1.00 | 34.51 | W | O |
| ATOM | 2323 | O | HOH | W | 11 | 11.509 | 26.503 | −1.609 | 1.00 | 24.67 | W | O |
| ATOM | 2324 | O | HOH | W | 12 | 4.068 | 30.522 | −11.763 | 1.00 | 30.45 | W | O |
| ATOM | 2325 | O | HOH | W | 13 | 7.206 | 36.754 | 8.481 | 1.00 | 28.49 | W | O |
| ATOM | 2326 | O | HOH | W | 14 | 24.856 | 47.149 | −11.673 | 1.00 | 36.87 | W | O |
| ATOM | 2327 | O | HOH | W | 15 | 30.041 | 25.070 | −0.613 | 1.00 | 28.85 | W | O |
| ATOM | 2328 | O | HOH | W | 16 | 7.804 | 39.907 | −4.499 | 1.00 | 29.84 | W | O |
| ATOM | 2329 | O | HOH | W | 17 | 19.195 | 42.824 | 11.432 | 1.00 | 23.73 | W | O |
| ATOM | 2330 | O | HOH | W | 18 | 6.029 | 35.904 | 1.295 | 1.00 | 32.21 | W | O |
| ATOM | 2331 | O | HOH | W | 19 | 32.052 | 13.285 | −12.059 | 1.00 | 43.10 | W | O |
| ATOM | 2332 | O | HOH | W | 20 | 30.535 | 27.128 | −2.441 | 1.00 | 36.26 | W | o |
| ATOM | 2333 | O | HOH | W | 21 | 23.962 | 41.086 | −15.791 | 1.00 | 33.42 | W | O |
| ATOM | 2334 | O | HOH | W | 22 | 14.853 | 47.840 | −11.833 | 1.00 | 27.61 | W | O |
| ATOM | 2335 | O | HOH | W | 23 | 6.116 | 31.381 | −10.205 | 1.00 | 31.05 | W | O |
| ATOM | 2336 | O | HOH | W | 24 | 17.746 | 14.311 | −6.644 | 1.00 | 33.07 | W | O |
| ATOM | 2337 | O | HOH | W | 25 | 8.417 | 27.795 | −2.249 | 1.00 | 35.12 | W | O |
| ATOM | 2338 | O | HOH | W | 26 | 0.869 | 31.7.36 | −11.288 | 1.00 | 34.67 | W | O |
| ATOM | 2339 | O | HOH | W | 27 | 13.031 | 25.416 | −3.919 | 1.00 | 31.11 | W | O |
| ATOM | 2340 | O | HOH | W | 28 | 10.552 | 28.209 | 15.464 | 1.00 | 44.08 | W | O |
| ATOM | 2341 | O | HOH | W | 29 | 6.047 | 32.847 | 1.762 | 1.00 | 32.15 | W | O |
| ATOM | 2342 | O | HOH | W | 30 | 28.653 | 27.676 | −4.452 | 1.00 | 33.50 | W | O |
| ATOM | 2343 | O | HOH | W | 31 | 12.680 | 18.023 | −3.373 | 1.00 | 35.86 | W | O |
| ATOM | 2344 | O | HOH | W | 32 | 12.248 | 49.285 | −12.196 | 1.00 | 34.47 | W | O |
| ATOM | 2345 | O | HOH | W | 33 | 22.844 | 25.229 | −4.874 | 1.00 | 34.91 | W | O |
| ATOM | 2346 | O | HOH | W | 34 | 18.041 | 30.547 | −11.568 | 1.00 | 35.72 | W | O |
| ATOM | 2347 | O | HOH | W | 35 | 0.359 | 30.834 | 3.181 | 1.00 | 37.12 | W | O |
| ATOM | 2348 | O | HOH | W | 36 | 12.464 | 21.249 | 4.826 | 1.00 | 32.13 | W | O |
| ATOM | 2349 | O | HOH | W | 37 | 31.088 | 42.980 | −8.204 | 1.00 | 33.32 | W | O |
| ATOM | 2350 | O | HOH | W | 38 | 5.264 | 35.914 | −3.440 | 1.00 | 33.62 | W | O |
| ATOM | 2351 | O | HOH | W | 39 | 17.371 | 17.662 | 7.030 | 1.00 | 40.85 | W | O |
| ATOM | 2352 | O | HOH | W | 40 | 29.961 | 37.594 | −17.391 | 1.00 | 43.48 | W | O |
| ATOM | 2353 | O | HOH | W | 41 | 18.158 | 23.617 | 13.470 | 1.00 | 42.33 | W | O |
| ATOM | 2354 | O | HOH | W | 42 | 22.984 | 51.388 | −8.847 | 1.00 | 36.61 | W | O |
| ATOM | 2355 | O | HOH | W | 43 | 24.405 | 27.822 | 14.803 | 1.00 | 31.01 | W | O |
| ATOM | 2356 | O | HOH | W | 44 | −4.934 | 31.704 | −2.402 | 1.00 | 32.46 | W | O |
| ATOM | 2357 | O | HOH | W | 45 | 25.084 | 14.109 | 3.129 | 1.00 | 48.04 | W | O |
| ATOM | 2358 | O | HOH | W | 46 | 20.063 | 53.568 | −6.478 | 1.00 | 32.40 | W | O |
| ATOM | 2359 | O | NOH | W | 47 | 22.061 | 48.313 | 5.634 | 1.00 | 36.00 | W | O |
| ATOM | 2360 | O | HOH | W | 48 | 10.187 | 22.802 | 14.864 | 1.00 | 37.95 | W | O |
| ATOM | 2361 | O | HOH | W | 49 | 0.008 | 13.070 | 2.114 | 1.00 | 45.21 | W | O |
| ATOM | 2362 | O | HOH | W | 50 | 25.041 | 14.480 | −2.610 | 1.00 | 45.49 | W | O |
| ATOM | 2363 | O | HOH | W | 51 | 6.656 | 46.258 | −0.949 | 1.00 | 39.79 | W | O |
| ATOM | 2364 | O | HOH | W | 52 | 13.822 | 50.139 | −0.359 | 1.00 | 40.63 | W | O |
| ATOM | 2365 | O | HOH | W | 53 | 22.387 | 53.214 | 2.600 | 1.00 | 38.29 | W | O |
| ATOM | 2366 | O | HOH | W | 54 | 9.153 | 26.472 | −4.218 | 1.00 | 36.65 | W | O |
| ATOM | 2367 | O | HOH | W | 55 | 14.610 | 31.184 | −9.251 | 1.00 | 31.27 | W | O |
| ATOM | 2368 | O | HOH | W | 56 | 24.935 | 17.603 | 8.567 | 1.00 | 44.53 | W | O |
| ATOM | 2369 | O | HOH | W | 57 | 7.252 | 46.276 | −4.053 | 1.00 | 39.45 | W | O |
| ATOM | 2370 | O | HOH | W | 58 | 25.818 | 41.981 | −17.191 | 1.00 | 40.37 | W | O |
| ATOM | 2371 | O | HOH | W | 59 | 28.022 | 40.016 | −17.228 | 1.00 | 48.52 | W | O |
| ATOM | 2372 | O | HOH | W | 60 | 29.274 | 48.054 | −6.225 | 1.00 | 44.84 | W | O |
| ATOM | 2373 | O | HOH | W | 61 | −5.269 | 19.566 | 7.820 | 1.00 | 56.98 | W | O |
| ATOM | 2374 | O | HOH | W | 62 | 2.640 | 24.211 | −5.163 | 1.00 | 50.27 | W | O |
| ATOM | 2375 | O | HOH | W | 63 | 7.923 | 20.900 | −1.169 | 1.00 | 37.33 | W | O |
| ATOM | 2376 | O | HOH | W | 64 | 27.634 | 45.162 | −13.207 | 1.00 | 46.05 | W | O |
| ATOM | 2377 | O | HOH | W | 65 | 7.070 | 38.833 | −10.815 | 1.00 | 35.97 | W | O |
| ATOM | 2378 | O | HOH | W | 66 | −1.437 | 33.817 | −10.585 | 1.00 | 42.27 | W.0 | |
| ATOM | 2379 | O | HOH | W | 67 | 2.293 | 29.053 | −0.105 | 1.00 | 43.18 | W | O |
| ATOM | 2380 | O | HOH | W | 68 | −0.337 | 29.845 | 0.902 | 1.00 | 44.30 | W | O |
| ATOM | 2381 | O | HOH | W | 69 | 13.627 | 17.597 | −11.581 | 1.00 | 52.67 | WO | |
| ATOM | 2382 | O | HOH | W | 70 | 10.652 | 22.799 | 4.564 | 1.00 | 49.65 | W | O |
| ATOM | 2383 | O | HOH | W | 71 | 32.699 | 40.511 | −10.516 | 1.00 | 53.08 | W | O |
| ATOM | 2384 | O | HOH | W | 72 | 30.236 | 43.692 | −12.518 | 1.00 | 48.54 | W | O |
| ATOM | 2385 | O | HOH | W | 73 | 19.844 | 27.263 | 13.669 | 1.00 | 44.13 | W | O |
| ATOM | 2386 | O | HOH | W | 74 | 17.734 | 29.535 | 16.425 | 1.00 | 42.59 | W | O |
| ATOM | 2387 | O | HOH | W | 75 | 7.234 | 19.806 | 7.154 | 1.00 | 46.54 | W | O |
| ATOM | 2388 | O | HOH | W | 76 | 26.364 | 11.064 | −2.697 | 1.00 | 41.27 | W | O |
| ATOM | 2389 | O | HOH | W | 77 | 9.100 | 50.578 | −6.099 | 1.00 | 36.36 | W | O |
| ATOM | 2390 | O | HOH | W | 78 | 11.854 | 52.633 | −2.732 | 1.00 | 53.20 | W | O |
| ATOM | 2391 | O | HOH | W | 79 | 11.974 | 56.050 | 1.375 | 1.00 | 76.32 | W | O |
| ATOM | 2392 | O | HOH | W | 80 | 22.479 | 50.601 | −11.573 | 1.00 | 47.12 | W | O |
| ATOM | 2393 | O | HOH | W | 81 | −0.004 | 0.007 | 6.072 | 1.00 | 38.70 | W | O |
| ATOM | 2394 | O | HOH | W | 82 | 4.477 | 41.554 | −4.606 | 1.00 | 34.09 | W | O |
| ATOM | 2395 | O | HOH | W | 83 | 27.838 | 53.344 | 0.486 | 1.00 | 48.36 | W | O |
| ATOM | 2396 | O | HOH | W | 84 | 22.017 | 53.906 | −8.187 | 1.00 | 38.28 | W | O |
| ATOM | 2397 | O | HOH | W | 85 | 12.617 | 34.402 | 19.138 | 1.00 | 41.13 | W | O |
| ATOM | 2398 | O | HOH | W | 86 | 2.912 | 32.727 | 4.894 | 1.00 | 51.51 | W | O |
| ATOM | 2399 | O | HOH | W | 87 | 27.534 | 26.168 | 5.351 | 1.00 | 31.51 | W | O |
| ATOM | 2400 | O | HOH | W | 88 | 11.125 | 12.829 | −7.881 | 1.00 | 52.64 | W | O |
| ATOM | 2401 | O | HOH | W | 89 | 21.024 | 21.470 | 12.494 | 1.00 | 41.27 | W | O |
| ATOM | 2402 | O | HOH | W | 90 | 14.012 | 14.000 | 14.848 | 1.00 | 47.62 | W | O |
| ATOM | 2403 | O | HOH | W | 91 | 11.506 | 10.890 | 14.020 | 1.00 | 42.83 | W | O |
| ATOM | 2404 | O | HOH | W | 92 | 1.120 | 31.537 | 10.417 | 1.00 | 42.16 | W | O |
| ATOM | 2405 | O | HOH | W | 93 | 4.751 | 32.938 | 3.780 | 1.00 | 42.70 | W | O |
| ATOM | 2406 | O | HOH | W | 94 | 19.088 | 36.501 | 11.901 | 1.00 | 40.49 | W | O |
| ATOM | 2407 | O | HOH | W | 95 | 23.080 | 30.887 | 11.698 | 1.00 | 32.51 | W | O |
| ATOM | 2408 | O | HOH | W | 96 | −3.355 | 27.300 | −3.494 | 1.00 | 40.37 | W | O |
| ATOM | 2409 | O | HOH | W | 97 | 6.415 | 47.469 | −17.497 | 1.00 | 45.34 | W | O |
| ATOM | 2410 | O | HOH | W | 98 | 27.847 | 44.555 | −15.690 | 1.00 | 43.12 | W | O |
| ATOM | 2411 | O | HOH | W | 99 | 21.740 | 53.159 | −2.940 | 1.00 | 45.77 | W | O |
| ATOM | 2412 | O | HOH | W | 100 | 34.748 | 28.537 | −3.258 | 1.00 | 33.58 | W | O |
| ATOM | 2413 | O | HOH | W | 101 | 32.169 | 31.691 | 5.630 | 1.00 | 49.99 | W | O |

TABLE 2
Coordinates of c-FMS (FGF-chimera) in complex with 793693 (Quinolone)

| | | | | | | | | | | | | | |
|---|---|---|---|---|---|---|---|---|---|---|---|---|---|
| ATOM | 1 | CB | VAL | A | 548 | 9.777 | 25.835 | −8.002 | 1.00 | 47.46 | A | C | |
| ATOM | 2 | CG1 | VAL | A | 548 | 8.868 | 25.978 | −6.788 | 1.00 | 47.60 | A | C | |
| ATOM | 3 | CG2 | VAL | A | 548 | 9.490 | 26.966 | −8.985 | 1.00 | 47.02 | A | C | |
| ATOM | 4 | C | VAL | A | 548 | 11.681 | 24.617 | −6.732 | 1.00 | 48.10 | A | C | |
| ATOM | 5 | O | VAL | A | 548 | 11.759 | 24.714 | −5.500 | 1.00 | 48.85 | A | O | |
| ATOM | 6 | N | VAL | A | 548 | 12.137 | 26.116 | −8.795 | 1.00 | 48.54 | A | N | |
| ATOM | 7 | CA | VAL | A | 548 | 11.286 | 25.876 | −7.583 | 1.00 | 48.22 | A | C | |
| ATOM | 8 | N | ARG | A | 549 | 11.950 | 23.462 | −7.364 | 1.00 | 46.82 | A | N | |
| ATOM | 9 | CA | ARG | A | 549 | 12.337 | 22.234 | −6.629 | 1.00 | 43.63 | A | C | |
| ATOM | 10 | CB | ARG | A | 549 | 11.770 | 20.963 | −7.295 | 1.00 | 43.39 | A | C | |
| ATOM | 11 | CG | ARG | A | 549 | 10.347 | 21.048 | −7.864 | 1.00 | 44.56 | A | C | |
| ATOM | 12 | CD | ARG | A | 549 | 9.277 | 21.106 | −6.786 | 1.00 | 46.33 | A | C | |
| ATOM | 13 | NE | ARG | A | 549 | 7.907 | 21.191 | −7.312 | 1.00 | 47.70 | A | N | |
| ATOM | 14 | CZ | ARG | A | 549 | 7.390 | 20.356 | −8.217 | 1.00 | 50.05 | A | C | |
| ATOM | 15 | NH1 | ARG | A | 549 | 8.134 | 19.371 | −8.719 | 1.00 | 50.64 | A | N | |
| ATOM | 16 | NH2 | ARG | A | 549 | 6.113 | 20.471 | −8.592 | 1.00 | 48.71 | A | N | |
| ATOM | 17 | C | ARG | A | 549 | 13.862 | 22.062 | −6.549 | 1.00 | 42.80 | A | C | |
| ATOM | 18 | O | ARG | A | 549 | 14.574 | 22.221 | −7.548 | 1.00 | 42.02 | A | O | |
| ATOM | 19 | N | TRP | A | 550 | 14.351 | 21.721 | −5.355 | 1.00 | 42.29 | A | N | |
| ATOM | 20 | CA | TRP | A | 550 | 15.781 | 21.487 | −5.106 | 1.00 | 40.36 | A | C | |
| ATOM | 21 | CB | TRP | A | 550 | 16.006 | 21.156 | −3.615 | 1.00 | 38.80 | A | C | |
| ATOM | 22 | CG | TRP | A | 550 | 16.042 | 22.368 | −2.722 | 1.00 | 36.46 | A | C | |
| ATOM | 23 | CD2 | TRP | A | 550 | 16.477 | 22.430 | −1.347 | 1.00 | 34.47 | A | C | |
| ATOM | 24 | CE2 | TRP | A | 550 | 16.435 | 23.793 | −0.956 | 1.00 | 33.72 | A | C | |
| ATOM | 25 | CE3 | TRP | A | 550 | 16.905 | 21.474 | −0.416 | 1.00 | 33.16 | A | C | |
| ATOM | 26 | CD1 | TRP | A | 550 | 15.751 | 23.657 | −3.087 | 1.00 | 36.01 | A | C | |
| ATOM | 27 | NE1 | TRP | A | 550 | 15.988 | 24.513 | −2.034 | 1.00 | 35.68 | A | N | |
| ATOM | 28 | CZ2 | TRP | A | 550 | 16.806 | 24.226 | 0.328 | 1.00 | 31.12 | A | C | |
| ATOM | 29 | CZ3 | TRP | A | 550 | 17.278 | 21.907 | 0.866 | 1.00 | 33.47 | A | C | |
| ATOM | 30 | CH2 | TRP | A | 550 | 17.226 | 23.279 | 1.221 | 1.00 | 31.47 | A | C | |
| ATOM | 31 | C | TRP | A | 550 | 16.208 | 20.319 | −5.980 | 1.00 | 40.36 | A | C | |
| ATOM | 32 | O | TRP | A | 550 | 15.378 | 19.524 | −6.371 | 1.00 | 40.45 | A | O | |
| ATOM | 33 | N | LYS | A | 551 | 17.493 | 20.192 | −6.282 | 1.00 | 42.69 | A | N | |
| ATOM | 34 | CA | LYS | A | 551 | 17.931 | 19.099 | −7.152 | 1.00 | 44.73 | A | C | |
| ATOM | 35 | CB | LYS | A | 551 | 17.579 | 19.456 | −8.604 | 1.00 | 46.90 | A | C | |
| ATOM | 36 | CG | LYS | A | 551 | 18.037 | 18.468 | −9.677 | 1.00 | 49.06 | A | C | |
| ATOM | 37 | CD | LYS | A | 551 | 17.445 | 18.874 | −11.027 | 1.00 | 50.07 | A | C | |
| ATOM | 38 | CE | LYS | A | 551 | 17.674 | 17.820 | −12.114 | 1.00 | 51.26 | A | C | |
| ATOM | 39 | NZ | LYS | A | 551 | 16.907 | 18.163 | −13.364 | 1.00 | 51.81 | A | N | |
| ATOM | 40 | C | LYS | A | 551 | 19.418 | 18.795 | −7.058 | 1.00 | 44.73 | A | C | |
| ATOM | 41 | O | LYS | A | 551 | 20.244 | 19.714 | −7.096 | 1.00 | 45.59 | A | O | |
| ATOM | 42 | N | ILE | A | 552 | 19.767 | 17.516 | −6.928 | 1.00 | 45.19 | A | N | |
| ATOM | 43 | CA | ILE | A | 552 | 21.182 | 17.141 | −6.877 | 1.00 | 45.37 | A | C | |
| ATOM | 44 | CB | ILE | A | 552 | 21.426 | 15.803 | −6.160 | 1.00 | 44.06 | A | C | |
| ATOM | 45 | CG2 | ILE | A | 552 | 22.931 | 15.518 | −6.120 | 1.00 | 44.22 | A | C | |
| ATOM | 46 | CG1 | ILE | A | 552 | 20.839 | 15.842 | −4.747 | 1.00 | 42.66 | A | C | |
| ATOM | 47 | CD1 | ILE | A | 552 | 21.480 | 16.846 | −3.850 | 1.00 | 41.98 | A | C | |
| ATOM | 48 | C | ILE | A | 552 | 21.617 | 16.969 | −8.329 | 1.00 | 46.39 | A | C | |
| ATOM | 49 | O | ILE | A | 552 | 20.859 | 16.433 | −9.149 | 1.00 | 47.88 | A | O | |
| ATOM | 50 | N | ILE | A | 553 | 22.820 | 17.429 | −8.654 | 1.00 | 46.14 | A | N | |
| ATOM | 51 | CA | ILE | A | 553 | 23.311 | 17.311 | −10.015 | 1.00 | 47.07 | A | C | |
| ATOM | 52 | CB | ILE | A | 553 | 23.498 | 18.692 | −10.671 | 1.00 | 45.77 | A | C | |
| ATOM | 53 | CG2 | ILE | A | 553 | 22.166 | 19.422 | −10.744 | 1.00 | 44.44 | A | C | |
| ATOM | 54 | CG1 | ILE | A | 553 | 24.515 | 19.511 | −9.884 | 1.00 | 44.08 | A | C | |
| ATOM | 55 | CD1 | ILE | A | 553 | 24.861 | 20.821 | −10.552 | 1.00 | 43.43 | A | C | |
| ATOM | 56 | C | ILE | A | 533 | 24.634 | 16.577 | −10.011 | 1.00 | 49.28 | A | C | |
| ATOM | 57 | O | ILE | A | 553 | 25.279 | 16.455 | −8.972 | 1.00 | 49.94 | A | O | |
| ATOM | 58 | N | GLU | A | 554 | 25.042 | 16.084 | −11.174 | 1.00 | 53.01 | A | N | |
| ATOM | 59 | CA | GLU | A | 554 | 26.298 | 15.347 | −11.266 | 1.00 | 56.51 | A | C | |
| ATOM | 60 | CB | GLU | A | 554 | 26.274 | 14.398 | −12.473 | 1.00 | 56.86 | A | C | |
| ATOM | 61 | CG | GLU | A | 554 | 24.962 | 13.622 | −12.621 | 1.00 | 57.21 | A | C | |
| ATOM | 62 | CD | GLU | A | 554 | 25.072 | 12.456 | −13.585 | 1.00 | 58.05 | A | C | |
| ATOM | 63 | OE1 | GLU | A | 554 | 25.672 | 12.623 | −14.677 | 1.00 | 58.33 | A | O | |
| ATOM | 64 | OE2 | GLU | A | 554 | 24.547 | 11.370 | −13.248 | 1.00 | 57.79 | A | O | |
| ATOM | 65 | C | GLU | A | 554 | 27.493 | 16.288 | −11.362 | 1.00 | 58.04 | A | C | |
| ATOM | 66 | O | GLU | A | 554 | 27.438 | 17.311 | −12.055 | 1.00 | 57.43 | A | O | |
| ATOM | 67 | N | SER | A | 555 | 28.571 | 15.932 | −10.662 | 1.00 | 60.36 | A | N | |
| ATOM | 68 | CA | SER | A | 555 | 29.785 | 16.742 | −10.657 | 1.00 | 62.16 | A | C | |
| ATOM | 69 | CB | SER | A | 555 | 29.444 | 18.152 | −10.159 | 1.00 | 61.27 | A | C | |
| ATOM | 70 | OG | SER | A | 555 | 30.603 | 18.952 | −10.040 | 1.00 | 62.36 | A | O | |
| ATOM | 71 | C | SER | A | 555 | 30.941 | 16.168 | −9.816 | 1.00 | 63.44 | A | C | |
| ATOM | 72 | O | SER | A | 555 | 30.801 | 15.160 | −9.111 | 1.00 | 63.42 | A | O | |
| ATOM | 73 | N | TYR | A | 556 | 32.085 | 16.836 | −9.919 | 1.00 | 64.74 | A | N | |
| ATOM | 74 | CA | TYR | A | 556 | 33.304 | 16.502 | −9.186 | 1.00 | 65.12 | A | C | |
| ATOM | 75 | CB | TYR | A | 556 | 34.198 | 15.582 | −10.024 | 1.00 | 67.00 | A | C | |
| ATOM | 76 | CG | TYR | A | 556 | 34.653 | 16.207 | −11.319 | 1.00 | 69.41 | A | C | |
| ATOM | 77 | CD1 | TYR | A | 556 | 35.629 | 17.212 | −11.329 | 1.00 | 70.07 | A | C | |
| ATOM | 78 | CE1 | TYR | A | 556 | 36.015 | 17.839 | −12.513 | 1.00 | 71.36 | A | C | |
| ATOM | 79 | CD2 | TYR | A | 556 | 34.074 | 15.835 | −12.535 | 1.00 | 70.87 | A | C | |
| ATOM | 80 | CE2 | TYR | A | 556 | 34.455 | 16.453 | −13.730 | 1.00 | 72.35 | A | C | |
| ATOM | 81 | CZ | TYR | A | 556 | 35.425 | 17.457 | −13.710 | 1.00 | 72.66 | A | C | |
| ATOM | 82 | OH | TYR | A | 556 | 35.796 | 18.083 | −14.884 | 1.00 | 74.24 | A | O | |
| ATOM | 83 | C | TYR | A | 556 | 33.988 | 17.857 | −8.969 | 1.00 | 64.79 | A | C | |
| ATOM | 84 | O | TYR | A | 556 | 33.788 | 18.787 | −9.754 | 1.00 | 65.23 | A | O | |
| ATOM | 85 | N | GLU | A | 557 | 34.782 | 17.987 | −7.915 | 1.00 | 63.66 | A | N | |
| ATOM | 86 | CA | GLU | A | 557 | 34.455 | 19.255 | −7.661 | 1.00 | 61.63 | A | C | |
| ATOM | 87 | CB | GLU | A | 557 | 35.870 | 19.333 | −6.197 | 1.00 | 60.62 | A | C | |
| ATOM | 88 | CG | GLU | A | 557 | 34.701 | 19.154 | −5.258 | 1.00 | 60.14 | A | C | |
| ATOM | 89 | CD | GLU | A | 557 | 35.123 | 18.907 | −3.825 | 1.00 | 60.19 | A | C | |
| ATOM | 90 | OE1 | GLU | A | 557 | 35.710 | 19.824 | −3.199 | 1.00 | 59.18 | A | O | |
| ATOM | 91 | OE2 | GLU | A | 557 | 34.864 | 17.786 | −3.330 | 1.00 | 59.67 | A | O | |
| ATOM | 92 | C | GLU | A | 557 | 36.670 | 19.402 | −8.565 | 1.00 | 61.26 | A | C | |
| ATOM | 93 | O | GLU | A | 557 | 36.530 | 19.611 | −9.769 | 1.00 | 60.42 | A | O | |
| ATOM | 94 | N | SER | A | 560 | 31.488 | 18.870 | −4.386 | 1.00 | 44.83 | A | N | |
| ATOM | 95 | CA | SER | A | 560 | 32.184 | 17.628 | −4.054 | 1.00 | 44.18 | A | C | |
| ATOM | 96 | CB | SER | A | 560 | 31.650 | 17.052 | −2.734 | 1.00 | 45.04 | A | C | |
| ATOM | 97 | OG | SER | A | 560 | 32.563 | 16.115 | −2.176 | 1.00 | 44.63 | A | O | |
| ATOM | 98 | C | SER | A | 560 | 31.980 | 16.618 | −5.186 | 1.00 | 42.67 | A | C | |
| ATOM | 99 | O | SER | A | 560 | 31.951 | 16.994 | −6.363 | 1.00 | 41.27 | A | O | |
| ATOM | 100 | N | TYR | A | 561 | 31.861 | 15.340 | −4.830 | 1.00 | 40.78 | A | N | |
| ATOM | 101 | CA | TYR | A | 561 | 31.635 | 14.307 | −5.830 | 1.00 | 39.98 | A | C | |
| ATOM | 102 | CB | TYR | A | 561 | 32.786 | 13.295 | −5.857 | 1.00 | 38.19 | A | C | |
| ATOM | 103 | CG | TYR | A | 561 | 32.658 | 12.257 | −6.962 | 1.00 | 36.14 | A | C | |
| ATOM | 104 | CD1 | TYR | A | 561 | 33.644 | 12.119 | −7.948 | 1.00 | 33.88 | A | C | |
| ATOM | 105 | CE1 | TYR | A | 561 | 33.532 | 11.145 | −8.958 | 1.00 | 31.64 | A | C | |
| ATOM | 106 | CD2 | TYR | A | 561 | 31.554 | 11.396 | −7.014 | 1.00 | 37.03 | A | C | |
| ATOM | 107 | CE2 | TYR | A | 561 | 31.437 | 10.421 | −8.021 | 1.00 | 34.86 | A | C | |
| ATOM | 108 | CZ | TYR | A | 561 | 32.425 | 10.306 | −8.984 | 1.00 | 32.03 | A | C | |
| ATOM | 109 | OH | TYR | A | 561 | 32.263 | 9.373 | −9.975 | 1.00 | 30.08 | A | O | |
| ATOM | 110 | C | TYR | A | 561 | 30.327 | 13.593 | −5.534 | 1.00 | 40.35 | A | C | |
| ATOM | 111 | O | TYR | A | 561 | 30.097 | 13.103 | −4.422 | 1.00 | 40.31 | A | O | |
| ATOM | 112 | N | THR | A | 562 | 29.469 | 13.537 | −6.542 | 1.00 | 40.36 | A | N | |
| ATOM | 113 | CA | THR | A | 562 | 28.178 | 12.889 | −6.407 | 1.00 | 40.36 | A | C | |
| ATOM | 114 | CB | THR | A | 562 | 27.117 | 13.651 | −7.265 | 1.00 | 40.33 | A | C | |
| ATOM | 115 | OG1 | THR | A | 562 | 26.669 | 14.811 | −6.539 | 1.00 | 40.09 | A | O | |
| ATOM | 116 | CG2 | THR | A | 562 | 25.929 | 12.752 | −7.619 | 1.00 | 40.05 | A | C | |
| ATOM | 117 | C | THR | A | 562 | 28.283 | 11.407 | −6.806 | 1.00 | 40.23 | A | C | |
| ATOM | 118 | O | THR | A | 562 | 28.352 | 11.082 | −7.990 | 1.00 | 39.80 | A | O | |
| ATOM | 119 | N | PHE | A | 563 | 28.318 | 10.527 | −5.800 | 1.00 | 40.51 | A | N | |
| ATOM | 120 | CA | PHE | A | 563 | 28.408 | 9.075 | −6.001 | 1.00 | 40.62 | A | C | |
| ATOM | 121 | CB | PHE | A | 563 | 29.058 | 8.371 | −4.798 | 1.00 | 38.90 | A | C | |
| ATOM | 122 | CG | PHE | A | 563 | 30.479 | 8.769 | −4.553 | 1.00 | 39.05 | A | C | |
| ATOM | 123 | CD1 | PHE | A | 563 | 30.785 | 9.778 | −3.643 | 1.00 | 39.01 | A | C | |
| ATOM | 124 | CD2 | PHE | A | 563 | 31.514 | 8.167 | −5.268 | 1.00 | 38.88 | A | C | |
| ATOM | 125 | CE1 | PHE | A | 563 | 32.093 | 10.186 | −3.450 | 1.00 | 38.37 | A | C | |
| ATOM | 126 | CE2 | PHE | A | 563 | 32.825 | 8.567 | −5.084 | 1.00 | 38.04 | A | C | |
| ATOM | 127 | CZ | PHE | A | 563 | 33.114 | 9.583 | −4.171 | 1.00 | 39.34 | A | C | |
| ATOM | 128 | C | PHE | A | 563 | 27.027 | 8.465 | −6.193 | 1.00 | 42.16 | A | C | |
| ATOM | 129 | O | PHE | A | 563 | 26.900 | 7.333 | −6.679 | 1.00 | 42.38 | A | O | |
| ATOM | 130 | N | ILE | A | 564 | 25.998 | 9.200 | −5.776 | 1.00 | 43.21 | A | N | |
| ATOM | 131 | CA | ILE | A | 564 | 24.629 | 8.721 | −5.908 | 1.00 | 44.39 | A | C | |
| ATOM | 132 | CB | ILE | A | 564 | 24.142 | 7.935 | −4.643 | 1.00 | 42.82 | A | C | |
| ATOM | 133 | CG2 | ILE | A | 564 | 25.089 | 6.799 | −4.317 | 1.00 | 43.12 | A | C | |
| ATOM | 134 | CG1 | ILE | A | 564 | 24.004 | 8.886 | −3.456 | 1.00 | 40.80 | A | C | |
| ATOM | 135 | CD1 | ILE | A | 564 | 23.426 | 8.240 | −2.231 | 1.00 | 39.56 | A | C | |
| ATOM | 136 | C | ILE | A | 564 | 23.637 | 9.858 | −6.112 | 1.00 | 46.88 | A | C | |
| ATOM | 137 | O | ILE | A | 564 | 23.855 | 11.002 | −5.688 | 1.00 | 47.99 | A | O | |
| ATOM | 138 | N | ASP | A | 565 | 22.535 | 9.520 | −6.764 | 1.00 | 48.46 | A | N | |
| ATOM | 139 | CA | ASP | A | 565 | 21.461 | 10.463 | −6.988 | 1.00 | 49.02 | A | C | |
| ATOM | 140 | CB | ASP | A | 565 | 21.251 | 10.685 | −8.473 | 1.00 | 49.70 | A | C | |
| ATOM | 141 | CG | ASP | A | 565 | 20.153 | 11.680 | −8.739 | 1.00 | 52.38 | A | C | |
| ATOM | 142 | OD1 | ASP | A | 565 | 18.971 | 11.340 | −8.483 | 1.00 | 50.74 | A | O | |
| ATOM | 143 | OD2 | ASP | A | 565 | 20.480 | 12.813 | −9.179 | 1.00 | 53.81 | A | O | |
| ATOM | 144 | C | ASP | A | 565 | 20.222 | 9.806 | −6.380 | 1.00 | 49.28 | A | C | |
| ATOM | 145 | O | ASP | A | 565 | 19.585 | 8.981 | −7.035 | 1.00 | 49.57 | A | O | |
| ATOM | 146 | N | PRO | A | 566 | 19.864 | 10.171 | −5.123 | 1.00 | 49.29 | A | N | |
| ATOM | 147 | CD | PRO | A | 566 | 20.420 | 11.388 | −4.487 | 1.00 | 48.41 | A | C | |
| ATOM | 148 | CA | PRO | A | 566 | 18.720 | 9.667 | −4.339 | 1.00 | 48.67 | A | C | |
| ATOM | 149 | CB | PRO | A | 566 | 18.314 | 10.886 | −3.519 | 1.00 | 47.63 | A | C | |
| ATOM | 150 | CG | PRO | A | 566 | 19.671 | 11.458 | −3.160 | 1.00 | 47.76. | A | C | |
| ATOM | 151 | C | PRO | A | 566 | 17.537 | 9.042 | −5.100 | 1.00 | 49.01 | A | C | |
| ATOM | 152 | O | PRO | A | 566 | 16.392 | 9.477 | −4.952 | 1.00 | 48.50 | A | O | |
| ATOM | 153 | N | THR | A | 567 | 17.828 | 8.011 | −5.895 | 1.00 | 49.02 | A | N | |
| ATOM | 154 | CA | THR | A | 567 | 16.822 | 7.296 | −6.680 | 1.00 | 48.69 | A | C | |
| ATOM | 155 | CB | THR | A | 567 | 17.453 | 6.646 | −7.955 | 1.00 | 48.16 | A | C | |
| ATOM | 156 | OG1 | THR | A | 567 | 17.888 | 7.670 | −8.862 | 1.00 | 47.36 | A | O | |
| ATOM | 157 | CG2 | THR | A | 567 | 16.441 | 5.745 | −8.661 | 1.00 | 47.85 | A | C | |
| ATOM | 158 | C | THR | A | 567 | 16.228 | 6.194 | −5.805 | 1.00 | 48.90 | A | C | |
| ATOM | 159 | O | THR | A | 567 | 16.848 | 5.774 | −4.820 | 1.00 | 48.98 | A | O | |
| ATOM | 160 | N | ASN | A | 572 | 16.451 | 6.958 | 4.066 | 1.00 | 58.95 | A | N | |
| ATOM | 161 | CA | ASN | A | 572 | 16.475 | 6.107 | 5.249 | 1.00 | 57.82 | A | C | |
| ATOM | 162 | CB | ASN | A | 572 | 17.113 | 6.842 | 6.433 | 1.00 | 58.19 | A | C | |
| ATOM | 163 | CG | ASN | A | 572 | 18.624 | 6.753 | 6.435 | 1.00 | 58.70 | A | C | |
| ATOM | 164 | OD1 | ASN | A | 572 | 19.189 | 5.659 | 6.499 | 1.00 | 58.34 | A | O | |
| ATOM | 165 | ND2 | ASN | A | 572 | 19.291 | 7.908 | 6.372 | 1.00 | 58.40 | A | N | |
| ATOM | 166 | C | ASN | A | 572 | 15.095 | 5.649 | 5.677 | 1.00 | 57.49 | A | C | |
| ATOM | 167 | O | ASN | A | 572 | 14.157 | 5.547 | 4.873 | 1.00 | 58.14 | A | O | |
| ATOM | 168 | N | GLU | A | 573 | 15.023 | 5.372 | 6.975 | 1.00 | 56.22 | A | N | |
| ATOM | 169 | CA | GLU | A | 573 | 13.840 | 4.933 | 7.693 | 1.00 | 55.42 | A | C | |
| ATOM | 170 | CB | GLU | A | 573 | 13.577 | 3.436 | 7.475 | 1.00 | 55.42 | A | C | |
| ATOM | 171 | CG | GLU | A | 573 | 12.363 | 2.870 | 8.251 | 1.00 | 57.51 | A | C | |
| ATOM | 172 | CD | GLU | A | 573 | 11.243 | 2.305 | 7.350 | 1.00 | 58.82 | A | C | |
| ATOM | 173 | QE1 | GLU | A | 573 | 11.519 | 1.419 | 6.497 | 1.00 | 58.32 | A | O | |
| ATOM | 174 | OE2 | GLU | A | 573 | 10.074 | 2.742 | 7.508 | 1.00 | 58.48 | A | O | |
| ATOM | 175 | C | GLU | A | 573 | 14.269 | 5.212 | 9.132 | 1.00 | 55.07 | A | C | |
| ATOM | 176 | O | GLU | A | 573 | 13.623 | 4.805 | 10.101 | 1.00 | 55.41 | A | O | |
| ATOM | 177 | N | LYS | A | 574 | 15.392 | 5.915 | 9.249 | 1.00 | 54.11 | A | N | |
| ATOM | 178 | CA | LYS | A | 574 | 15.936 | 6.297 | 10.541 | 1.00 | 53.36 | A | C | |
| ATOM | 179 | CB | LYS | A | 574 | 17.467 | 6.325 | 10.499 | 1.00 | 53.22 | A | C | |
| ATOM | 180 | CG | LYS | A | 574 | 18.053 | 7.361 | 9.549 | 1.00 | 53.68 | A | C | |
| ATOM | 181 | CD | LYS | A | 574 | 19.329 | 7.986 | 10.125 | 1.00 | 54.66 | A | C | |
| ATOM | 182 | CE | LYS | A | 574 | 19.886 | 9.112 | 9.243 | 1.00 | 54.48 | A | C | |
| ATOM | 183 | NZ | LYS | A | 574 | 20.749 | 10.058 | 10.020 | 1.00 | 52.12 | A | N | |
| ATOM | 184 | C | LYS | A | 574 | 15.413 | 7.693 | 10.865 | 1.00 | 53.43 | A | C | |
| ATOM | 185 | O | LYS | A | 574 | 15.749 | 8.276 | 11.905 | 1.00 | 53.22 | A | O | |
| ATOM | 186 | N | TRP | A | 575 | 14.595 | 8.225 | 9.957 | 1.00 | 52.72 | A | N | |
| ATOM | 187 | CA | TRP | A | 575 | 14.011 | 9.557 | 10.124 | 1.00 | 52.04 | A | C | |
| ATOM | 188 | CB | TRP | A | 575 | 13.969 | 10.293 | 8.774 | 1.00 | 50.58 | A | C | |
| ATOM | 189 | CG | TRP | A | 575 | 15.311 | 10.802 | 8.333 | 1.00 | 48.44 | A | C | |
| ATOM | 190 | CD2 | TRP | A | 575 | 16.035 | 11.912 | 8.890 | 1.00 | 48.04 | A | C | |
| ATOM | 191 | CE2 | TRP | A | 575 | 17.270 | 11.990 | 8.208 | 1.00 | 47.26 | A | C | |
| ATOM | 192 | CE3 | TRP | A | 575 | 15.759 | 12.847 | 9.902 | 1.00 | 47.70 | A | C | |
| ATOM | 193 | CD1 | TRP | A | 575 | 16.111 | 10.273 | 7.364 | 1.00 | 48.56 | A | C | |
| ATOM | 194 | NE1 | TRP | A | 575 | 17.289 | 10.979 | 7.283 | 1.00 | 48.02 | A | N | |
| ATOM | 195 | CZ2 | TRP | A | 575 | 18.233 | 12.965 | 8.504 | 1.00 | 46.38 | A | C | |
| ATOM | 196 | CZ3 | TRP | A | 575 | 16.720 | 13.819 | 10.196 | 1.00 | 47.71 | A | C | |
| ATOM | 197 | CH2 | TRP | A | 575 | 17.943 | 13.866 | 9.497 | 1.00 | 46.13 | A | C | |
| ATOM | 198 | C | TRP | A | 575 | 12.603 | 9.452 | 10.697 | 1.00 | 51.94 | A | C | |
| ATOM | 199 | O | TRP | A | 575 | 12.035 | 10.430 | 11.207 | 1.00 | 50.44 | A | O | |
| ATOM | 200 | N | GLU | A | 576 | 12.067 | 8.237 | 10.614 | 1.00 | 52.48 | A | N | |
| ATOM | 201 | CA | GLU | A | 576 | 10.728 | 7.927 | 11.082 | 1.00 | 52.63 | A | C | |
| ATOM | 202 | CB | GLU | A | 576 | 10.487 | 6.432 | 11.026 | 1.00 | 50.30 | A | C | |
| ATOM | 203 | CG | GLU | A | 576 | 9.047 | 6.087 | 10.804 | 1.00 | 47.68 | A | C | |
| ATOM | 204 | CD | GLU | A | 576 | 8.559 | 6.501 | 9.438 | 1.00 | 46.73 | A | C | |
| ATOM | 205 | OE1 | GLU | A | 576 | 9.402 | 6.664 | 8.513 | 1.00 | 45.59 | A | O | |
| ATOM | 206 | OE2 | GLU | A | 576 | 7.323 | 6.643 | 9.293 | 1.00 | 46.12 | A | O | |
| ATOM | 207 | C | GLU | A | 576 | 10.457 | 8.410 | 12.492 | 1.00 | 54.23 | A | C | |
| ATOM | 208 | O | GLU | A | 576 | 11.362 | 8.499 | 13.330 | 1.00 | 53.34 | A | O | |
| ATOM | 209 | N | PHE | A | 577 | 9.188 | 8.704 | 12.740 | 1.00 | 55.65 | A | N | |
| ATOM | 210 | CA | PHE | A | 577 | 8.745 | 9.199 | 14.030 | 1.00 | 57.24 | A | C | |
| ATOM | 211 | CB | PHE | A | 577 | 8.933 | 10.720 | 14.075 | 1.00 | 57.19 | A | C | |
| ATOM | 212 | CG | PHE | A | 577 | 8.445 | 11.364 | 15.338 | 1.00 | 57.52 | A | C | |
| ATOM | 213 | CD1 | PHE | A | 577 | 9.063 | 11.103 | 16.552 | 1.00 | 58.05 | A | C | |
| ATOM | 214 | CD2 | PHE | A | 577 | 7.363 | 12.240 | 15.310 | 1.00 | 57.93 | A | C | |
| ATOM | 215 | CE1 | PHE | A | 577 | 8.607 | 11.709 | 17.725 | 1.00 | 59.37 | A | C | |
| ATOM | 216 | CE2 | PHE | A | 577 | 6.899 | 12.852 | 16.474 | 1.00 | 57.78 | A | C | |
| ATOM | 217 | CZ | PHE | A | 577 | 7.521 | 12.586 | 17.683 | 1.00 | 58.40 | A | C | |
| ATOM | 218 | C | PHE | A | 577 | 7.270 | 8.821 | 14.183 | 1.00 | 58.45 | A | C | |
| ATOM | 219 | O | PHE | A | 577 | 6.499 | 8.897 | 13.216 | 1.00 | 58.06 | A | O | |
| ATOM | 220 | N | PRO | A | 578 | 6.870 | 8.378 | 15.393 | 1.00 | 59.30 | A | N | |
| ATOM | 221 | CD | PRO | A | 578 | 7.732 | 8.207 | 16.579 | 1.00 | 59.32 | A | C | |
| ATOM | 222 | CA | PRO | A | 578 | 5.485 | 7.981 | 15.691 | 1.00 | 59.55 | A | C | |
| ATOM | 223 | CB | PRO | A | 578 | 5.545 | 7.629 | 17.176 | 1.00 | 60.03 | A | C | |
| ATOM | 224 | CG | PRO | A | 578 | 6.985 | 7.172 | 17.367 | 1.00 | 59.74 | A | C | |
| ATOM | 225 | C | PRO | A | 578 | 4.585 | 9.180 | 15.420 | 1.00 | 60.11 | A | C | |
| ATOM | 226 | O | PRO | A | 578 | 4.831 | 10.259 | 15.945 | 1.00 | 60.70 | A | O | |
| ATOM | 227 | N | ARG | A | 579 | 3.543 | 9.009 | 14.620 | 1.00 | 60.91 | A | N | |
| ATOM | 228 | CA | ARG | A | 579 | 2.697 | 10.148 | 14.296 | 1.00 | 62.33 | A | C | |
| ATOM | 229 | CB | ARG | A | 579 | 1.984 | 9.909 | 12.973 | 1.00 | 61.81 | A | C | |
| ATOM | 230 | CG | ARG | A | 579 | 0.759 | 9.052 | 13.055 | 1.00 | 62.38 | A | C | |
| ATOM | 231 | CD | ARG | A | 579 | 0.142 | 8.998 | 11.686 | 1.00 | 62.86 | A | C | |
| ATOM | 232 | NE | ARG | A | 579 | 1.062 | 8.365 | 10.749 | 1.00 | 64.66 | A | N | |
| ATOM | 233 | CZ | ARG | A | 579 | 1.077 | 8.591 | 9.440 | 1.00 | 65.17 | A | C | |
| ATOM | 234 | NH1 | ARG | A | 579 | 0.226 | 9.450 | 8.897 | 1.00 | 64.30 | A | N | |
| ATOM | 235 | NH2 | ARG | A | 579 | 1.925 | 7.930 | 8.665 | 1.00 | 65.79 | A | N | |
| ATOM | 236 | C | ARG | A | 579 | 1.683 | 10.550 | 15.355 | 1.00 | 63.97 | A | C | |
| ATOM | 237 | O | ARG | A | 579 | 0.859 | 11.438 | 15.126 | 1.00 | 64.19 | A | O | |
| ATOM | 238 | N | ASN | A | 580 | 1.742 | 9.906 | 16.515 | 1.00 | 65.85 | A | N | |
| ATOM | 239 | CA | ASN | A | 580 | 0.823 | 10.233 | 17.596 | 1.00 | 67.58 | A | C | |
| ATOM | 240 | CB | ASN | A | 580 | 0.098 | 8.979 | 18.079 | 1.00 | 66.76 | A | C | |
| ATOM | 241 | CG | ASN | A | 580 | 1.053 | 7.880 | 18.463 | 1.00 | 67.04 | A | C | |
| ATOM | 242 | OD1 | ASN | A | 580 | 1.964 | 8.093 | 19.266 | 1.00 | 66.33 | A | O | |
| ATOM | 243 | ND2 | ASN | A | 580 | 0.857 | 6.691 | 17.890 | 1.00 | 67.17 | A | N | |
| ATOM | 244 | C | ASN | A | 580 | 1.564 | 10.897 | 18.759 | 1.00 | 69.12 | A | C | |
| ATOM | 245 | O | ASN | A | 580 | 1.013 | 11.033 | 19.856 | 1.00 | 70.57 | A | O | |
| ATOM | 246 | N | ASN | A | 581 | 2.819 | 11.287 | 18.517 | 1.00 | 69.76 | A | N | |
| ATOM | 247 | CA | ASN | A | 581 | 3.645 | 11.993 | 19.512 | 1.00 | 70.25 | A | C | |
| ATOM | 248 | CB | ASN | A | 581 | 4.951 | 11.252 | 19.801 | 1.00 | 71.22 | A | C | |
| ATOM | 249 | CG | ASN | A | 581 | 4.721 | 9.853 | 20.290 | 1.00 | 73.11 | A | C | |
| ATOM | 250 | OD1 | ASN | A | 581 | 4.214 | 9.004 | 19.550 | 1.00 | 73.82 | A | O | |
| ATOM | 251 | ND2 | ASN | A | 581 | 5.083 | 9.595 | 21.546 | 1.00 | 73.82 | A | N | |
| ATOM | 252 | C | ASN | A | 581 | 3.984 | 13.328 | 18.868 | 1.00 | 69.49 | A | C | |
| ATOM | 253 | O | ASN | A | 581 | 5.005 | 13.952 | 19.176 | 1.00 | 69.49 | A | O | |
| ATOM | 254 | N | LEU | A | 582 | 3.098 | 13.742 | 17.969 | 1.00 | 67.91 | A | N | |
| ATOM | 255 | CA | LEU | A | 582 | 3.243 | 14.962 | 17.204 | 1.00 | 66.65 | A | C | |
| ATOM | 256 | CB | LEU | A | 582 | 3.602 | 14.564 | 15.770 | 1.00 | 66.90 | A | C | |
| ATOM | 257 | CG | LEU | A | 582 | 4.363 | 15.480 | 14.821 | 1.00 | 66.79 | A | C | |
| ATOM | 258 | CD1 | LEU | A | 582 | 5.697 | 15.862 | 15.441 | 1.00 | 67.13 | A | C | |
| ATOM | 259 | CD2 | LEU | A | 582 | 4.573 | 14.743 | 13.496 | 1.00 | 66.19 | A | C | |
| ATOM | 260 | C | LEU | A | 582 | 1.896 | 15.687 | 17.240 | 1.00 | 65.55 | A | C | |
| ATOM | 261 | O | LEU | A | 582 | 0.967 | 15.301 | 16.527 | 1.00 | 65.82 | A | O | |
| ATOM | 262 | N | GLN | A | 583 | 1.773 | 16.725 | 18.062 | 1.00 | 64.23 | A | N | |
| ATOM | 263 | CA | GLN | A | 583 | 0.500 | 17.435 | 18.134 | 1.00 | 63.98 | A | C | |
| ATOM | 264 | CB | GLN | A | 583 | 0.148 | 17.793 | 19.584 | 1.00 | 65.93 | A | C | |
| ATOM | 265 | CG | GLN | A | 583 | −1.172 | 18.574 | 19.695 | 1.00 | 68.34 | A | C | |
| ATOM | 266 | CD | GLN | A | 583 | −1.557 | 18.936 | 21.127 | 1.00 | 70.63 | A | C | |
| ATOM | 267 | OE1 | GLN | A | 583 | −2.477 | 19.735 | 21.352 | 1.00 | 71.24 | A | O | |
| ATOM | 268 | NE2 | GLN | A | 583 | −0.861 | 18.345 | 22.102 | 1.00 | 72.41 | A | N | |
| ATOM | 269 | C | GLN | A | 583 | 0.445 | 18.693 | 17.274 | 1.00 | 62.44 | A | C | |
| ATOM | 270 | O | GLN | A | 583 | 1.289 | 19.582 | 17.401 | 1.00 | 61.40 | A | O | |
| ATOM | 271 | N | PHE | A | 584 | −0.573 | 18.753 | 16.414 | 1.00 | 60.79 | A | N | |
| ATOM | 272 | CA | PHE | A | 584 | −0.784 | 19.877 | 15.508 | 1.00 | 59.46 | A | C | |
| ATOM | 273 | CB | PHE | A | 584 | −2.076 | 19.696 | 14.702 | 1.00 | 59.96 | A | C | |
| ATOM | 274 | CG | PHE | A | 584 | −1.999 | 18.639 | 13.620 | 1.00 | 61.31 | A | C | |
| ATOM | 275 | CD1 | PHE | A | 584 | −1.425 | 17.389 | 13.872 | 1.00 | 61.49 | A | C | |
| ATOM | 276 | CD2 | PHE | A | 584 | −2.594 | 18.864 | 12.373 | 1.00 | 60.40 | A | C | |
| ATOM | 277 | CE1 | PHE | A | 584 | −1.451 | 16.377 | 12.898 | 1.00 | 61.69 | A | C | |
| ATOM | 278 | CE2 | PHE | A | 584 | −2.628 | 17.863 | 11.397 | 1.00 | 60.75 | A | C | |
| ATOM | 279 | CZ | PHE | A | 584 | −2.057 | 16.615 | 11.657 | 1.00 | 60.92 | A | C | |
| ATOM | 280 | C | PHE | A | 584 | −0.869 | 21.223 | 16.214 | 1.00 | 58.55 | A | C | |
| ATOM | 281 | O | PHE | A | 584 | −1.059 | 21.310 | 17.428 | 1.00 | 58.42 | A | O | |
| ATOM | 282 | N | GLY | A | 585 | −0.724 | 22.270 | 15.410 | 1.00 | 57.90 | A | N | |
| ATOM | 283 | CA | GLY | A | 585 | −0.806 | 23.645 | 15.868 | 1.00 | 55.24 | A | C | |
| ATOM | 284 | C | GLY | A | 585 | −1.573 | 24.289 | 14.732 | | 1.00 | 54.21 | A | C |
| ATOM | 285 | O | GLY | A | 585 | −2.255 | 23.575 | 13.984 | 1.00 | 53.50 | A | O | |
| ATOM | 286 | N | LYS | A | 586 | −1.477 | 25.602 | 14.564 | 1.00 | 52.61 | A | N | |
| ATOM | 287 | CA | LYS | A | 586 | −2.206 | 26.217 | 13.462 | 1.00 | 52.03 | A | C | |
| ATOM | 288 | CB | LYS | A | 586 | −2.344 | 27.731 | 13.675 | 1.00 | 52.50 | A | C | |
| ATOM | 289 | CG | LYS | A | 586 | −1.043 | 28.494 | 13.699 | 1.00 | 53.47 | A | C | |
| ATOM | 290 | CD | LYS | A | 586 | −1.283 | 29.919 | 14.146 | 1.00 | 54.15 | A | C | |
| ATOM | 291 | CE | LYS | A | 586 | 0.020 | 30.699 | 14.236 | 1.00 | 56.06 | A | C | |
| ATOM | 292 | NZ | LYS | A | 586 | 1.019 | 30.064 | 15.148 | 1.00 | 56.51 | A | N | |
| ATOM | 293 | C | LYS | A | 586 | −1.523 | 25.928 | 12.123 | 1.00 | 50.75 | A | C | |
| ATOM | 294 | O | LYS | A | 586 | −0.380 | 25.454 | 12.088 | 1.00 | 51.32 | A | O | |
| ATOM | 295 | N | THR | A | 587 | −2.245 | 26.192 | 11.032 | 1.00 | 49.11 | A | N | |
| ATOM | 296 | CA | THR | A | 587 | −1.743 | 26.004 | 9.664 | 1.00 | 46.40 | A | C | |
| ATOM | 297 | CB | THR | A | 587 | −2.912 | 25.829 | 8.668 | 1.00 | 44.62 | A | C | |
| ATOM | 298 | OG1 | THR | A | 587 | −3.251 | 24.438 | 8.559 | 1.00 | 43.84 | A | O | |
| ATOM | 299 | CG2 | THR | A | 587 | −2.543 | 26.379 | 7.295 | 1.00 | 44.21 | A | C | |
| ATOM | 300 | C | THR | A | 587 | −0.937 | 27.249 | 9.267 | 1.00 | 45.63 | A | C | |
| ATOM | 301 | O | THR | A | 587 | −1.513 | 28.311 | 9.027 | 1.00 | 46.78 | A | O | |
| ATOM | 302 | N | LEU | A | 588 | 0.385 | 27.117 | 9.199 | 1.00 | 43.21 | A | N | |
| ATOM | 303 | CA | LEU | A | 588 | 1.254 | 28.244 | 8.862 | 1.00 | 40.58 | A | C | |
| ATOM | 304 | CB | LEU | A | 588 | 2.717 | 27.839 | 9.047 | 1.00 | 39.80 | A | C | |
| ATOM | 305 | CG | LEU | A | 588 | 3.055 | 27.211 | 10.397 | 1.00 | 38.97 | A | C | |
| ATOM | 306 | CD1 | LEU | A | 588 | 4.500 | 26.733 | 10.408 | 1.00 | 38.34 | A | C | |
| ATOM | 307 | CD2 | LEU | A | 588 | 2.821 | 28.239 | 11.486 | 1.00 | 39.66 | A | C | |
| ATOM | 308 | C | LEU | A | 588 | 1.047 | 28.744 | 7.434 | 1.00 | 38.98 | A | C | |
| ATOM | 309 | O | LEU | A | 588 | 1.135 | 29.944 | 7.157 | 1.00 | 39.08 | A | O | |
| ATOM | 310 | N | GLY | A | 589 | 0.776 | 27.824 | 6.522 | 1.00 | 36.15 | A | N | |
| ATOM | 311 | CA | GLY | A | 589 | 0.582 | 28.224 | 5.148 | 1.00 | 32.79 | A | C | |
| ATOM | 312 | C | GLY | A | 589 | 0.148 | 27.029 | 4.337 | 1.00 | 31.77 | A | C | |
| ATOM | 313 | O | GLY | A | 589 | 0.288 | 25.876 | 4.781 | 1.00 | 31.23 | A | O | |
| ATOM | 314 | N | ALA | A | 590 | −0.385 | 27.298 | 3.149 | 1.00 | 29.67 | A | N | |
| ATOM | 315 | CA | ALA | A | 590 | 0.842 | 26.236 | 2.284 | 1.00 | 29.14 | A | C | |
| ATOM | 316 | CB | ALA | A | 590 | −2.239 | 25.825 | 2.664 | 1.00 | 28.16 | A | C | |
| ATOM | 317 | C | ALA | A | 590 | −0.815 | 26.705 | 0.850 | 1.00 | 30.86 | A | C | |
| ATOM | 318 | O | ALA | A | 590 | −0.976 | 27.899 | 0.569 | 1.00 | 30.01 | A | O | |
| ATOM | 319 | N | GLY | A | 591 | −0.599 | 25.752 | −0.055 | 1.00 | 32.99 | A | N | |
| ATOM | 320 | CA | GLY | A | 591 | −0.563 | 26.045 | −1.474 | 1.00 | 33.79 | A | C | |
| ATOM | 321 | C | GLY | A | 591 | −1.517 | 25.121 | −2.205 | 1.00 | 34.76 | A | C | |
| ATOM | 322 | O | GLY | A | 591 | −2.200 | 24.295 | −1.589 | 1.00 | 35.27 | A | O | |
| ATOM | 323 | N | ALA | A | 592 | 1.549 | 25.247 | −3.525 | 1.00 | 35.51 | A | N | |
| ATOM | 324 | CA | ALA | A | 592 | −2.423 | 24.442 | −4.349 | 1.00 | 36.91 | A | C | |
| ATOM | 325 | CB | ALA | A | 592 | −2.563 | 25.108 | −5.701 | 1.00 | 39.07 | A | C | |
| ATOM | 326 | C | ALA | A | 592 | −1.946 | 22.993 | −4.517 | 1.00 | 38.33 | A | C | |
| ATOM | 327 | O | ALA | A | 592 | −2.138 | 22.385 | −5.581 | 1.00 | 38.44 | A | O | |
| ATOM | 328 | N | PHE | A | 593 | −1.339 | 22.435 | −3.473 | 1.00 | 39.11 | A | N | |
| ATOM | 329 | CA | PHE | A | 593 | −0.829 | 21.066 | −3.529 | 1.00 | 40.25 | A | C | |
| ATOM | 330 | CB | PHE | A | 593 | 0.586 | 21.056 | −4.110 | 1.00 | 42.24 | A | C | |
| ATOM | 331 | CG | PHE | A | 593 | 0.638 | 20.813 | −5.590 | 1.00 | 45.96 | A | C | |
| ATOM | 332 | CD1 | PHE | A | 593 | 1.795 | 21.106 | −6.311 | 1.00 | 46.96 | A | C | |
| ATOM | 333 | CD2 | PHE | A | 593 | −0.459 | 20.270 | −6.266 | 1.00 | 47.22 | A | C | |
| ATOM | 334 | CE1 | PHE | A | 593 | 1.870 | 20.862 | −7.690 | 1.00 | 48.43 | A | C | |
| ATOM | 335 | CE2 | PHE | A | 593 | −0.400 | 20.021 | −7.644 | 1.00 | 48.72 | A | C | |
| ATOM | 336 | CZ | PHE | A | 593 | 0.771 | 20.319 | −8.360 | 1.00 | 49.10 | A | C | |
| ATOM | 337 | C | PHE | A | 593 | −0.792 | 20.426 | −2.155 | 1.00 | 40.13 | A | C | |
| ATOM | 338 | O | PHE | A | 593 | −0.903 | 19.200 | −2.021 | 1.00 | 39.70 | A | O | |
| ATOM | 339 | N | GLY | A | 594 | −0.621 | 21.269 | −1.142 | 1.00 | 39.49 | A | N | |
| ATOM | 340 | CA | GLY | A | 594 | −0.548 | 20.789 | 0.222 | 1.00 | 39.74 | A | C | |
| ATOM | 341 | C | GLY | A | 594 | −0.464 | 21.955 | 1.192 | 1.00 | 40.32 | A | C | |
| ATOM | 342 | O | GLY | A | 594 | −0.508 | 23.125 | 0.791 | 1.00 | 40.45 | A | O | |
| ATOM | 343 | N | LYS | A | 595 | −0.347 | 21.642 | 2.476 | 1.00 | 39.54 | A | N | |
| ATOM | 344 | CA | LYS | A | 595 | −0.278 | 22.675 | 3.490 | 1.00 | 40.16 | A | C | |
| ATOM | 345 | CB | LYS | A | 595 | −1.602 | 22.776 | 4.242 | 1.00 | 41.91 | A | C | |
| ATOM | 346 | CG | LYS | A | 595 | −1.976 | 21.529 | 5.037 | 1.00 | 43.05 | A | C | |
| ATOM | 347 | CD | LYS | A | 595 | −3.276 | 21.800 | 5.821 | 1.00 | 45.55 | A | C | |
| ATOM | 348 | CE | LYS | A | 595 | −3.783 | 20.584 | 6.600 | 1.00 | 43.58 | A | C | |
| ATOM | 349 | NZ | LYS | A | 595 | −4.944 | 20.960 | 7.462 | 1.00 | 42.35 | A | N | |
| ATOM | 350 | C | LYS | A | 595 | 0.807 | 22.309 | 4.461 | 1.00 | 40.18 | A | C | |
| ATOM | 351 | O | LYS | A | 595 | 1.177 | 21.145 | 4.586 | 1.00 | 40.62 | A | O | |
| ATOM | 352 | N | VAL | A | 596 | 1.321 | 23.305 | 5.158 | 1.00 | 39.05 | A | N | |
| ATOM | 353 | CA | VAL | A | 596 | 2.367 | 23.054 | 6.117 | 1.00 | 39.09 | A | C | |
| ATOM | 354 | CB | VAL | A | 596 | 3.705 | 23.672 | 5.612 | 1.00 | 38.09 | A | C | |
| ATOM | 355 | CG1 | VAL | A | 596 | 3.446 | 25.032 | 5.008 | 1.00 | 39.37 | A | C | |
| ATOM | 356 | CG2 | VAL | A | 596 | 4.715 | 23.770 | 6.740 | 1.00 | 37.48 | A | C | |
| ATOM | 357 | C | VAL | A | 596 | 1.886 | 23.662 | 7.433 | 1.00 | 39.87 | A | C | |
| ATOM | 358 | O | VAL | A | 596 | 1.573 | 24.858 | 7.501 | 1.00 | 39.54 | A | O | |
| ATOM | 359 | N | VAL | A | 597 | 1.791 | 22.837 | 8.474 | 1.00 | 39.98 | A | N | |
| ATOM | 360 | CA | VAL | A | 597 | 1.314 | 23.344 | 9.757 | 1.00 | 41.26 | A | C | |
| ATOM | 361 | CB | VAL | A | 597 | −0.004 | 22.681 | 10.162 | 1.00 | 40.40 | A | C | |
| ATOM | 362 | CG1 | VAL | A | 597 | −1.027 | 22.886 | 9.074 | 1.00 | 41.23 | A | C | |
| ATOM | 363 | CG2 | VAL | A | 597 | 0.217 | 21.196 | 10.430 | 1.00 | 40.26 | A | C | |
| ATOM | 364 | C | VAL | A | 597 | 2.266 | 23.207 | 10.933 | 1.00 | 41.81 | A | C | |
| ATOM | 365 | O | VAL | A | 597 | 3.124 | 22.315 | 10.984 | 1.00 | 41.25 | A | O | |
| ATOM | 366 | N | GLU | A | 598 | 2.100 | 24.108 | 11.889 | 1.00 | 42.35 | A | N | |
| ATOM | 367 | CA | GLU | A | 598 | 2.927 | 24.077 | 13.076 | 1.00 | 43.71 | A | C | |
| ATOM | 368 | CB | GLU | A | 598 | 2.648 | 25.305 | 13.948 | 1.00 | 45.75 | A | C | |
| ATOM | 369 | CG | GLU | A | 598 | 3.489 | 25.375 | 15.204 | 1.00 | 49.66 | A | C | |
| ATOM | 370 | CD | GLU | A | 598 | 3.257 | 26.654 | 15.986 | 1.00 | 52.68 | A | C | |
| ATOM | 371 | OE1 | GLU | A | 598 | 2.102 | 26.908 | 16.403 | 1.00 | 54.25 | A | O | |
| ATOM | 372 | OE2 | GLU | A | 598 | 4.235 | 27.408 | 16.179 | 1.00 | 54.95 | A | O | |
| ATOM | 373 | C | GLU | A | 598 | 2.512 | 22.811 | 13.790 | 1.00 | 42.85 | A | C | |
| ATOM | 374 | O | GLU | A | 598 | 1.522 | 22.184 | 13.407 | 1.00 | 42.66 | A | O | |
| ATOM | 375 | N | ALA | A | 599 | 3.267 | 22.429 | 14.812 | 1.00 | 42.36 | A | N | |
| ATOM | 376 | CA | ALA | A | 599 | 2.954 | 21.235 | 15.577 | 1.00 | 42.59 | A | C | |
| ATOM | 377 | CB | ALA | A | 599 | 2.879 | 20.029 | 14.655 | 1.00 | 42.42 | A | C | |
| ATOM | 378 | C | ALA | A | 599 | 4.013 | 21.028 | 16.638 | 1.00 | 43.17 | A | C | |
| ATOM | 379 | O | ALA | A | 599 | 5.175 | 21.363 | 16.429 | 1.00 | 42.92 | A | O | |
| ATOM | 380 | N | THR | A | 600 | 3.604 | 20.484 | 17.783 | 1.00 | 45.74 | A | N | |
| ATOM | 381 | CA | THR | A | 600 | 4.527 | 20.239 | 18.896 | 1.00 | 46.98 | A | C | |
| ATOM | 382 | CB | THR | A | 600 | 3.827 | 20.428 | 20.255 | 1.00 | 46.38 | A | C | |
| ATOM | 383 | OG1 | THR | A | 600 | 2.967 | 21.569 | 20.187 | 1.00 | 46.88 | A | O | |
| ATOM | 384 | CG2 | THR | A | 600 | 4.852 | 20.650 | 21.359 | 1.00 | 45.90 | A | C | |
| ATOM | 385 | C | THR | A | 600 | 5.044 | 18.814 | 18.816 | 1.00 | 47.64 | A | C | |
| ATOM | 386 | O | THR | A | 600 | 4.269 | 17.878 | 18.625 | 1.00 | 48.05 | A | O | |
| ATOM | 387 | N | ALA | A | 601 | 6.355 | 18.657 | 18.956 | 1.00 | 48.89 | A | N | |
| ATOM | 388 | CA | ALA | A | 601 | 6.972 | 17.340 | 18.886 | 1.00 | 50.15 | A | C | |
| ATOM | 389 | CB | ALA | A | 601 | 8.255 | 17.402 | 18.096 | 1.00 | 51.30 | A | C | |
| ATOM | 390 | C | ALA | A | 601 | 7.267 | 16.865 | 20.275 | 1.00 | 51.62 | A | C | |
| ATOM | 391 | O | ALA | A | 601 | 7.709 | 17.643 | 21.118 | 1.00 | 52.75 | A | O | |
| ATOM | 392 | N | PHE | A | 602 | 7.032 | 15.581 | 20.512 | 1.00 | 53.24 | A | N | |
| ATOM | 393 | CA | PHE | A | 602 | 7.273 | 15.008 | 21.828 | 1.00 | 53.71 | A | C | |
| ATOM | 394 | CB | PHE | A | 602 | 6.000 | 14.316 | 22.358 | 1.00 | 53.99 | A | C | |
| ATOM | 395 | CG | PHE | A | 602 | 4.799 | 15.235 | 22.453 | 1.00 | 54.08 | A | C | |
| ATOM | 396 | CD1 | PHE | A | 602 | 3.647 | 14.981 | 21.698 | 1.00 | 54.14 | A | C | |
| ATOM | 397 | CD2 | PHE | A | 602 | 4.832 | 16.370 | 23.263 | 1.00 | 53.77 | A | C | |
| ATOM | 398 | CE1 | PHE | A | 602 | 2.538 | 15.847 | 21.738 | 1.00 | 54.26 | A | C | |
| ATOM | 399 | CE2 | PHE | A | 602 | 3.736 | 17.241 | 23.313 | 1.00 | 55.38 | A | C | |
| ATOM | 400 | CZ | PHE | A | 602 | 2.579 | 16.976 | 22.542 | 1.00 | 54.52 | A | C | |
| ATOM | 401 | C | PHE | A | 602 | 8.440 | 14.030 | 21.773 | 1.00 | 53.26 | A | C | |
| ATOM | 402 | O | PHE | A | 602 | 8.308 | 12.894 | 21.314 | 1.00 | 52.77 | A | O | |
| ATOM | 403 | N | GLY | A | 603 | 9.592 | 14.494 | 22.236 | 1.00 | 53.76 | A | N | |
| ATOM | 404 | CA | GLY | A | 603 | 10.761 | 13.645 | 22.242 | 1.00 | 55.58 | A | C | |
| ATOM | 405 | C | GLY | A | 603 | 11.318 | 13.418 | 20.855 | 1.00 | 57.09 | A | C | |
| ATOM | 406 | O | GLY | A | 603 | 11.137 | 12.347 | 20.263 | 1.00 | 57.32 | A | C | |
| ATOM | 407 | N | LEU | A | 604 | 11.988 | 14.436 | 20.329 | 1.00 | 57.92 | A | N | |
| ATOM | 408 | CA | LEU | A | 604 | 12.584 | 14.334 | 19.012 | 1.00 | 59.02 | A | C | |
| ATOM | 409 | CB | LEU | A | 604 | 12.107 | 15.474 | 18.105 | 1.00 | 58.01 | A | C | |
| ATOM | 410 | CG | LEU | A | 604 | 11.282 | 15.020 | 16.888 | 1.00 | 57.50 | A | C | |
| ATOM | 411 | CD1 | LEU | A | 604 | 10.666 | 16.223 | 16.194 | 1.00 | 56.71 | A | C | |
| ATOM | 412 | CD2 | LEU | A | 604 | 12.164 | 14.242 | 15.918 | 1.00 | 56.60 | A | C | |
| ATOM | 413 | C | LEU | A | 604 | 14.092 | 14.367 | 19.171 | 1.00 | 60.28 | A | C | |
| ATOM | 414 | O | LEU | A | 604 | 14.651 | 15.298 | 19.764 | 1.00 | 59.64 | A | O | |
| ATOM | 415 | N | GLY | A | 605 | 14.739 | 13.321 | 18.659 | 1.00 | 61.92 | A | N | |
| ATOM | 416 | CA | GLY | A | 605 | 16.184 | 13.217 | 18.742 | 1.00 | 63.58 | A | C | |
| ATOM | 417 | C | GLY | A | 605 | 16.667 | 12.315 | 19.863 | 1.00 | 64.29 | A | C | |
| ATOM | 418 | O | GLY | A | 605 | 15.884 | 11.746 | 20.628 | 1.00 | 63.59 | A | O | |
| ATOM | 419 | N | LYS | A | 606 | 17.983 | 12.190 | 19.956 | 1.00 | 65.90 | A | N | |
| ATOM | 420 | CA | LYS | A | 606 | 18.606 | 11.363 | 20.976 | 1.00 | 67.72 | A | C | |
| ATOM | 421 | CB | LYS | A | 606 | 20.119 | 11.279 | 20.694 | 1.00 | 67.82 | A | C | |
| ATOM | 422 | CG | LYS | A | 606 | 20.848 | 10.088 | 21.315 | 1.00 | 66.78 | A | C | |
| ATOM | 423 | CD | LYS | A | 606 | 21.378 | 10.394 | 22.708 | 1.00 | 66.18 | A | C | |
| ATOM | 424 | CE | LYS | A | 606 | 22.560 | 11.349 | 22.663 | 1.00 | 65.29 | A | C | |
| ATOM | 425 | NZ | LYS | A | 606 | 23.132 | 11.567 | 24.023 | 1.00 | 63.98 | A | N | |
| ATOM | 426 | C | LYS | A | 606 | 18.342 | 11.996 | 22.345 | 1.00 | 68.60 | A | C | |
| ATOM | 427 | O | LYS | A | 606 | 18.651 | 11.412 | 23.383 | 1.00 | 68.46 | A | O | |
| ATOM | 428 | N | GLU | A | 607 | 17.738 | 13.181 | 22.341 | 1.00 | 69.61 | A | N | |
| ATOM | 429 | CA | GLU | A | 607 | 17.482 | 13.892 | 23.585 | 1.00 | 71.49 | A | C | |
| ATOM | 430 | CB | GLU | A | 607 | 18.165 | 15.265 | 23.539 | 1.00 | 73.40 | A | C | |
| ATOM | 431 | CG | GLU | A | 607 | 19.685 | 15.208 | 23.440 | 1.00 | 75.48 | A | C | |
| ATOM | 432 | CD | GLU | A | 607 | 20.297 | 14.230 | 24.432 | 1.00 | 77.59 | A | C | |
| ATOM | 433 | OE1 | GLU | A | 607 | 19.823 | 14.169 | 25.593 | 1.00 | 78.50 | A | O | |
| ATOM | 434 | OE2 | GLU | A | 607 | 21.260 | 13.527 | 24.050 | 1.00 | 78.23 | A | O | |
| ATOM | 435 | C | GLU | A | 607 | 16.026 | 14.075 | 24.028 | 1.00 | 71.67 | A | C | |
| ATOM | 436 | O | GLU | A | 607 | 15.752 | 14.862 | 24.940 | 1.00 | 71.66 | A | O | |
| ATOM | 437 | N | ASP | A | 608 | 15.096 | 13.367 | 23.398 | 1.00 | 71.22 | A | N | |
| ATOM | 438 | CA | ASP | A | 608 | 13.683 | 13.468 | 23.778 | 1.00 | 70.84 | A | C | |
| ATOM | 439 | CB | ASP | A | 608 | 13.469 | 12.893 | 25.180 | 1.00 | 71.01 | A | C | |
| ATOM | 440 | CG | ASP | A | 608 | 14.065 | 11.509 | 25.341 | 1.00 | 72.07 | A | C | |
| ATOM | 441 | OD1 | ASP | A | 608 | 13.887 | 10.920 | 26.429 | 1.00 | 72.52 | A | O | |
| ATOM | 442 | OD2 | ASP | A | 608 | 14.715 | 11.017 | 24.387 | 1.00 | 72.73 | A | O | |
| ATOM | 443 | C | ASP | A | 608 | 13.118 | 14.888 | 23.755 | 1.00 | 70.11 | A | C | |
| ATOM | 444 | O | ASP | A | 608 | 12.034 | 15.132 | 24.294 | 1.00 | 69.90 | A | O | |
| ATOM | 445 | N | ALA | A | 609 | 13.847 | 15.815 | 23.138 | 1.00 | 69.11 | A | N | |
| ATOM | 446 | CA | ALA | A | 609 | 13.411 | 17.206 | 23.054 | 1.00 | 67.51 | A | C | |
| ATOM | 447 | CB | ALA | A | 609 | 14.217 | 17.943 | 21.991 | 1.00 | 66.81 | A | C | |
| ATOM | 448 | C | ALA | A | 609 | 11.922 | 17.291 | 22.733 | 1.00 | 66.71 | A | C | |
| ATOM | 449 | O | ALA | A | 609 | 11.384 | 16.464 | 21.998 | 1.00 | 66.61 | A | O | |
| ATOM | 450 | N | VAL | A | 610 | 11.259 | 18.290 | 23.301 | 1.00 | 65.54 | A | N | |
| ATOM | 451 | CA | VAL | A | 610 | 9.839 | 18.490 | 23.063 | 1.00 | 64.41 | A | C | |
| ATOM | 452 | CB | VAL | A | 610 | 9.028 | 18.321 | 24.369 | 1.00 | 64.82 | A | C | |
| ATOM | 453 | CG1 | VAL | A | 610 | 7.540 | 18.440 | 24.083 | 1.00 | 65.29 | A | C | |
| ATOM | 454 | CG2 | VAL | A | 610 | 9.336 | 16.971 | 24.995 | 1.00 | 65.20 | A | C | |
| ATOM | 455 | C | VAL | A | 610 | 9.646 | 19.903 | 22.522 | 1.00 | 63.24 | A | C | |
| ATOM | 456 | O | VAL | A | 610 | 9.097 | 20.776 | 23.205 | 1.00 | 63.99 | A | O | |
| ATOM | 457 | N | LEU | A | 611 | 10.104 | 20.127 | 21.292 | 1.00 | 60.87 | A | N | |
| ATOM | 458 | CA | LEU | A | 611 | 9.992 | 21.447 | 20.680 | 1.00 | 58.34 | A | C | |
| ATOM | 459 | CB | LEU | A | 611 | 11.371 | 21.925 | 20.194 | 1.00 | 58.56 | A | C | |
| ATOM | 460 | CG | LEU | A | 611 | 12.337 | 20.912 | 19.567 | 1.00 | 58.25 | A | C | |
| ATOM | 461 | CD1 | LEU | A | 611 | 11.676 | 20.162 | 18.403 | 1.00 | 57.90 | A | C | |
| ATOM | 462 | CD2 | LEU | A | 611 | 13.578 | 21.661 | 19.106 | 1.00 | 57.40 | A | C | |
| ATOM | 463 | C | LEU | A | 611 | 8.973 | 21.587 | 19.552 | 1.00 | 55.71 | A | C | |
| ATOM | 464 | O | LEU | A | 611 | 8.323 | 20.619 | 19.139 | 1.00 | 54.40 | A | O | |
| ATOM | 465 | N | LYS | A | 612 | 8.841 | 22.824 | 19.078 | 1.00 | 53.29 | A | N | |
| ATOM | 466 | CA | LYS | A | 612 | 7.918 | 23.172 | 18.004 | 1.00 | 51.02 | A | C | |
| ATOM | 467 | CB | LYS | A | 612 | 7.635 | 24.681 | 18.005 | 1.00 | 51.20 | A | C | |
| ATOM | 468 | CG | LYS | A | 612 | 6.199 | 25.086 | 18.351 | 1.00 | 51.42 | A | C | |
| ATOM | 469 | CD | LYS | A | 612 | 6.067 | 26.608 | 18.289 | 1.00 | 52.85 | A | C | |
| ATOM | 470 | CE | LYS | A | 612 | 4.751 | 27.130 | 18.878 | 1.00 | 52.48 | A | C | |
| ATOM | 471 | NZ | LYS | A | 612 | 4.673 | 28.625 | 18.765 | 1.00 | 49.96 | A | N | |
| ATOM | 472 | C | LYS | A | 612 | 8.530 | 22.788 | 16.676 | 1.00 | 48.82 | A | C | |
| ATOM | 473 | O | LYS | A | 612 | 9.726 | 22.982 | 16.448 | 1.00 | 48.95 | A | O | |
| ATOM | 474 | N | VAL | A | 613 | 7.706 | 22.239 | 15.799 | 1.00 | 46.61 | A | N | |
| ATOM | 475 | CA | VAL | A | 613 | 8.179 | 21.838 | 14.493 | 1.00 | 45.17 | A | C | |
| ATOM | 476 | CB | VAL | A | 613 | 8.383 | 20.305 | 14.395 | 1.00 | 44.41 | A | C | |
| ATOM | 477 | CG1 | VAL | A | 613 | 9.247 | 19.838 | 15.546 | 1.00 | 43.71 | A | C | |
| ATOM | 478 | CG2 | VAL | A | 613 | 7.040 | 19.577 | 14.379 | 1.00 | 42.13 | A | C | |
| ATOM | 479 | C | VAL | A | 613 | 7.156 | 22.253 | 13.471 | 1.00 | 44.74 | A | C | |
| ATOM | 480 | O | VAL | A | 613 | 6.039 | 22.644 | 13.818 | 1.00 | 44.06 | A | O | |
| ATOM | 481 | N | ALA | A | 614 | 7.552 | 22.169 | 12.205 | 1.00 | 44.55 | A | N | |
| ATOM | 482 | CA | ALA | A | 614 | 6.673 | 22.515 | 11.092 | 1.00 | 42.54 | A | C | |
| ATOM | 483 | CB | ALA | A | 614 | 7.304 | 23.596 | 10.224 | 1.00 | 42.26 | A | C | |
| ATOM | 484 | C | ALA | A | 614 | 6.499 | 21.238 | 10.301 | 1.00 | 40.83 | A | C | |
| ATOM | 485 | O | ALA | A | 614 | 7.470 | 20.627 | 9.852 | 1.00 | 40.31 | A | O | |
| ATOM | 486 | N | VAL | A | 615 | 5.254 | 20.826 | 10.144 | 1.00 | 39.66 | A | N | |
| ATOM | 487 | CA | VAL | A | 615 | 4.978 | 19.609 | 9.430 | 1.00 | 38.18 | A | C | |
| ATOM | 488 | CB | VAL | A | 615 | 3.877 | 18.811 | 10.146 | 1.00 | 37.39 | A | C | |
| ATOM | 489 | CG1 | VAL | A | 615 | 3.451 | 17.617 | 9.311 | 1.00 | 36.78 | A | C | |
| ATOM | 490 | CG2 | VAL | A | 615 | 4.394 | 18.345 | 11.506 | 1.00 | 37.52 | A | C | |
| ATOM | 491 | C | VAL | A | 615 | 4.562 | 19.887 | 8.008 | 1.00 | 39.09 | A | C | |
| ATOM | 492 | O | VAL | A | 615 | 3.507 | 20.485 | 7.770 | 1.00 | 40.21 | A | O | |
| ATOM | 493 | N | LYS | A | 616 | 5.416 | 19.487 | 7.067 | 1.00 | 39.16 | A | N | |
| ATOM | 494 | CA | LYS | A | 616 | 5.126 | 19.633 | 5.649 | 1.00 | 38.90 | A | C | |
| ATOM | 495 | CB | LYS | A | 616 | 6.370 | 19.359 | 4.792 | 1.00 | 38.33 | A | C | |
| ATOM | 496 | CG | LYS | A | 616 | 7.209 | 20.560 | 4.349 | 1.00 | 37.04 | A | C | |
| ATOM | 497 | CD | LYS | A | 616 | 8.050 | 20.163 | 3.120 | 1.00 | 37.18 | A | C | |
| ATOM | 498 | CE | LYS | A | 616 | 8.839 | 21.329 | 2.484 | 1.00 | 37.18 | A | C | |
| ATOM | 499 | NZ | LYS | A | 616 | 10.179 | 21.640 | 3.122 | 1.00 | 35.27 | A | N | |
| ATOM | 500 | C | LYS | A | 616 | 4.168 | 18.475 | 5.460 | 1.00 | 40.85 | A | C | |
| ATOM | 501 | O | LYS | A | 616 | 4.290 | 17.473 | 6.175 | 1.00 | 40.89 | A | O | |
| ATOM | 502 | N | MET | A | 617 | 3.226 | 18.605 | 4.525 | 1.00 | 43.10 | A | N | |
| ATOM | 503 | CA | MET | A | 617 | 2.257 | 17.540 | 4.217 | 1.00 | 45.85 | A | C | |
| ATOM | 504 | CB | MET | A | 617 | 1.320 | 17.257 | 5.400 | 1.00 | 46.75 | A | C | |
| ATOM | 505 | CG | MET | A | 617 | 0.179 | 18.263 | 5.530 | 1.00 | 47.63 | A | C | |
| ATOM | 506 | SD | MET | A | 617 | −1.019 | 17.872 | 6.812 | 1.00 | 47.39 | A | S | |
| ATOM | 507 | CE | MET | A | 617 | −0.012 | 18.106 | 8.348 | 1.00 | 43.61 | A | C | |
| ATOM | 508 | C | MET | A | 617 | 1.394 | 17.908 | 3.010 | 1.00 | 47.58 | A | C | |
| ATOM | 509 | O | MET | A | 617 | 1.268 | 19.082 | 2.643 | 1.00 | 46.47 | A | O | |
| ATOM | 510 | N | LEU | A | 618 | 0.785 | 16.896 | 2.403 | 1.00 | 50.18 | A | N | |
| ATOM | 511 | CA | LEU | A | 618 | −0.064 | 17.123 | 1.245 | 1.00 | 53.27 | A | C | |
| ATOM | 512 | CB | LEU | A | 618 | 0.184 | 16.051 | 0.187 | 1.00 | 51.71 | A | C | |
| ATOM | 513 | CG | LEU | A | 618 | 1.578 | 16.100 | −0.420 | 1.00 | 51.87 | A | C | |
| ATOM | 514 | CD1 | LEU | A | 618 | 1.844 | 14.812 | −1.170 | 1.00 | 52.80 | A | C | |
| ATOM | 515 | CD2 | LEU | A | 618 | 1.694 | 17.320 | −1.333 | 1.00 | 52.23 | A | C | |
| ATOM | 516 | C | LEU | A | 618 | −1.513 | 17.074 | 1.662 | 1.00 | 55.48 | A | C | |
| ATOM | 517 | O | LEU | A | 618 | −1.828 | 16.962 | 2.849 | 1.00 | 55.89 | A | O | |
| ATOM | 518 | N | LYS | A | 619 | −2.390 | 17.165 | 0.670 | 1.00 | 58.32 | A | N | |
| ATOM | 519 | CA | LYS | A | 619 | −3.821 | 17.094 | 0.901 | 1.00 | 60.54 | A | C | |
| ATOM | 520 | CB | LYS | A | 619 | −4.541 | 18.172 | 0.082 | 1.00 | 61.02 | A | C | |
| ATOM | 521 | CG | LYS | A | 619 | −4.258 | 19.599 | 0.552 | 1.00 | 61.12 | A | C | |
| ATOM | 522 | CD | LYS | A | 619 | −5.156 | 20.624 | −0.146 | 1.00 | 61.54 | A | C | |
| ATOM | 523 | CE | LYS | A | 619 | −4.935 | 22.033 | 0.423 | 1.00 | 61.49 | A | C | |
| ATOM | 524 | NZ | LYS | A | 619 | −5.770 | 23.074 | −0.237 | 1.00 | 59.44 | A | N | |
| ATOM | 525 | C | LYS | A | 619 | −4.283 | 15.698 | 0.482 | 1.00 | 62.04 | A | C | |
| ATOM | 526 | O | LYS | A | 619 | 3.468 | 14.850 | 0.096 | 1.00 | 63.25 | A | O | |
| ATOM | 527 | N | SER | A | 620 | −5.584 | 15.453 | 0.570 | 1.00 | 63.14 | A | N | |
| ATOM | 528 | CA | SER | A | 620 | −6.145 | 14.163 | 0.187 | 1.00 | 63.83 | A | C | |
| ATOM | 529 | CB | SER | A | 620 | −7.474 | 13.977 | 0.902 | 1.00 | 64.74 | A | C | |
| ATOM | 530 | OG | SER | A | 620 | −8.218 | 15.185 | 0.852 | 1.00 | 66.31 | A | O | |
| ATOM | 531 | C | SER | A | 620 | −6.342 | 14.140 | −1.327 | 1.00 | 64.02 | A | C | |
| ATOM | 532 | O | SER | A | 620 | −6.326 | 13.087 | −1.962 | 1.00 | 62.56 | A | O | |
| ATOM | 533 | N | THR | A | 621 | −6.517 | 15.329 | −1.890 | 1.00 | 65.98 | A | N | |
| ATOM | 534 | CA | THR | A | 621 | −6.709 | 15.512 | −3.323 | 1.00 | 68.26 | A | C | |
| ATOM | 535 | CB | THR | A | 621 | −6.813 | 17.019 | −3.674 | 1.00 | 69.69 | A | C | |
| ATOM | 536 | OG1 | THR | A | 621 | −7.756 | 17.653 | −2.797 | 1.00 | 70.32 | A | O | |
| ATOM | 537 | CG2 | THR | A | 621 | −7.263 | 17.209 | −5.129 | 1.00 | 70.36 | A | C | |
| ATOM | 538 | C | THR | A | 621 | −5.532 | 14.930 | −4.102 | 1.00 | 68.61 | A | C | |
| ATOM | 539 | O | THR | A | 621 | −5.666 | 14.567 | −5.271 | 1.00 | 68.73 | A | O | |
| ATOM | 540 | N | ALA | A | 622 | −4.381 | 14.854 | −3.442 | 1.00 | 69.45 | A | N | |
| ATOM | 541 | CA | ALA | A | 622 | −3.151 | 14.339 | −4.046 | 1.00 | 70.40 | A | C | |
| ATOM | 542 | CB | ALA | A | 622 | −2.124 | 14.066 | −2.951 | 1.00 | 69.26 | A | C | |
| ATOM | 543 | C | ALA | A | 622 | −3.324 | 13.085 | −4.907 | 1.00 | 71.40 | A | C | |
| ATOM | 544 | O | ALA | A | 622 | −4.400 | 12.475 | −4.953 | 1.00 | 71.17 | A | O | |
| ATOM | 545 | N | HIS | A | 623 | −2.249 | 12.718 | −5.601 | 1.00 | 72.42 | A | N | |
| ATOM | 546 | CA | HIS | A | 623 | −2.236 | 11.523 | −6.437 | 1.00 | 74.07 | A | C | |
| ATOM | 547 | CB | HIS | A | 623 | −3.010 | 11.731 | −7.745 | 1.00 | 75.62 | A | C | |
| ATOM | 548 | CG | HIS | A | 623 | −3.356 | 10.447 | −8.439 | 1.00 | 76.73 | A | C | |
| ATOM | 549 | CD2 | HIS | A | 623 | −4.522 | 9.998 | −8.959 | 1.00 | 77.03 | A | C | |
| ATOM | 550 | ND1 | HIS | A | 623 | −2.438 | 9.435 | −8.637 | 1.00 | 76.39 | A | N | |
| ATOM | 551 | CE1 | HIS | A | 623 | −3.023 | 8.421 | −9.247 | 1.00 | 76.34 | A | C | |
| ATOM | 552 | NE2 | HIS | A | 623 | −4.290 | 8.737 | −9.454 | 1.00 | 76.89 | A | N | |
| ATOM | 553 | C | HIS | A | 623 | −0.814 | 11.084 | −6.773 | 1.00 | 73.74 | A | C | |
| ATOM | 554 | O | HIS | A | 623 | 0.077 | 11.916 | −6.931 | 1.00 | 73.75 | A | O | |
| ATOM | 555 | N | ALA | A | 624 | −0.630 | 9.767 | −6.877 | 1.00 | 73.46 | A | N | |
| ATOM | 556 | CA | ALA | A | 624 | 0.651 | 9.135 | −7.193 | 1.00 | 72.27 | A | C | |
| ATOM | 557 | CB | ALA | A | 624 | 0.507 | 8.314 | −8.466 | 1.00 | 73.40 | A | C | |
| ATOM | 558 | C | ALA | A | 624 | 1.827 | 10.101 | −7.328 | 1.00 | 71.40 | A | C | |
| ATOM | 559 | O | ALA | A | 624 | 2.760 | 10.080 | −6.522 | 1.00 | 70.85 | A | O | |
| ATOM | 560 | N | ASP | A | 625 | 1.776 | 10.942 | −8.355 | 1.00 | 70.30 | A | N | |
| ATOM | 561 | CA | ASP | A | 625 | 2.828 | 11.917 | −8.602 | 1.00 | 69.00 | A | C | |
| ATOM | 562 | CB | ASP | A | 625 | 2.448 | 12.787 | −9.793 | 1.00 | 68.86 | A | C | |
| ATOM | 563 | CG | ASP | A | 625 | 2.087 | 11.965 | −11.013 | 1.00 | 69.75 | A | C | |
| ATOM | 564 | OD1 | ASP | A | 625 | 1.948 | 12.556 | −12.106 | 1.00 | 70.10 | A | 0 | |
| ATOM | 565 | OD2 | ASP | A | 625 | 1.937 | 10.725 | −10.878 | 1.00 | 69.40 | A | O | |
| ATOM | 566 | C | ASP | A | 625 | 3.089 | 12.784 | −7.374 | 1.00 | 68.17 | A | C | |
| ATOM | 567 | O | ASP | A | 625 | 4.127 | 12.635 | −6.726 | 1.00 | 68.35 | A | O | |
| ATOM | 568 | N | GLU | A | 626 | 2.150 | 13.677 | −7.050 | 1.00 | 67.26 | A | N | |
| ATOM | 569 | CA | GLU | A | 626 | 2.295 | 14.563 | −5.888 | 1.00 | 65.82 | A | C | |
| ATOM | 570 | CB | GLU | A | 626 | 0.939 | 15.134 | −5.420 | 1.00 | 66.32 | A | C | |
| ATOM | 571 | CG | GLU | A | 626 | −0.287 | 14.763 | −6.248 | 1.00 | 67.25 | A | C | |
| ATOM | 572 | CD | GLU | A | 626 | −0.413 | 15.574 | −7.521 | 1.00 | 67.65 | A | C | |
| ATOM | 573 | OE1 | GLU | A | 626 | 0.512 | 16.818 | −7.416 | 1.00 | 66.55 | A | O | |
| ATOM | 574 | OE2 | GLU | A | 626 | −0.420 | 14.966 | −8.620 | 1.00 | 68.59 | A | O | |
| ATOM | 575 | C | GLU | A | 626 | 2.924 | 13.810 | −4.718 | 1.00 | 64.43 | A | C | |
| ATOM | 576 | O | GLU | A | 626 | 3.774 | 14.347 | −4.006 | 1.00 | 64.33 | A | O | |
| ATOM | 577 | N | LYS | A | 627 | 2.498 | 12.566 | −4.524 | 1.00 | 62.26 | A | N | |
| ATOM | 578 | CA | LYS | A | 627 | 3.024 | 11.752 | −3.444 | 1.00 | 60.61 | A | C | |
| ATOM | 579 | CB | LYS | A | 627 | 2.289 | 10.413 | −3.390 | 1.00 | 60.13 | A | C | |
| ATOM | 580 | CG | LYS | A | 627 | 0.953 | 10.444 | −2.675 | 1.00 | 59.46 | A | C | |
| ATOM | 581 | CD | LYS | A | 627 | 0.360 | 9.041 | −2.585 | 1.00 | 59.44 | A | C | |
| ATOM | 582 | CE | LYS | A | 627 | −0.703 | 8.943 | −1.497 | 1.00 | 59.95 | A | C | |
| ATOM | 583 | NZ | LYS | A | 627 | −1.834 | 9.910 | −1.687 | 1.00 | 60.51 | A | N | |
| ATOM | 584 | C | LYS | A | 627 | 4.527 | 11.500 | −3.565 | 1.00 | 59.80 | A | C | |
| ATOM | 585 | O | LYS | A | 627 | 5.289 | 11.827 | −2.654 | 1.00 | 60.28 | A | O | |
| ATOM | 586 | N | GLU | A | 628 | 4.954 | 10.929 | −4.690 | 1.00 | 58.99 | A | N | |
| ATOM | 587 | CA | GLU | A | 628 | 6.366 | 10.606 | −4.894 | 1.00 | 58.12 | A | C | |
| ATOM | 588 | CB | GLU | A | 628 | 6.583 | 9.944 | −6.251 | 1.00 | 60.18 | A | C | |
| ATOM | 589 | CG | GLU | A | 628 | 7.448 | 8.689 | −6.174 | 1.00 | 62.35 | A | C | |
| ATOM | 590 | CD | GLU | A | 628 | 8.320 | 8.515 | −7.403 | 1.00 | 64.37 | A | C | |
| ATOM | 591 | OE1 | GLU | A | 628 | 9.312 | 9.274 | −7.539 | 1.00 | 64.18 | A | O | |
| ATOM | 592 | OE2 | GLU | A | 628 | 8.006 | 7.628 | −8.233 | 1.00 | 65.02 | A | O | |
| ATOM | 593 | C | GLU | A | 628 | 7.305 | 11.795 | −4.775 | 1.00 | 56.52 | A | C | |
| ATOM | 594 | O | GLU | A | 628 | 8.358 | 11.700 | −4.133 | 1.00 | 56.18 | A | O | |
| ATOM | 595 | N | ALA | A | 629 | 6.933 | 12.905 | −5.408 | 1.00 | 54.19 | A | N | |
| ATOM | 596 | CA | ALA | A | 629 | 7.746 | 14.116 | −5.354 | 1.00 | 51.42 | A | C | |
| ATOM | 597 | CB | ALA | A | 629 | 6.956 | 15.297 | −5.889 | 1.00 | 50.46 | A | C | |
| ATOM | 598 | C | ALA | A | 629 | 8.169 | 14.370 | −3.909 | 1.00 | 50.27 | A | C | |
| ATOM | 599 | O | ALA | A | 629 | 9.361 | 14.432 | −3.607 | 1.00 | 50.38 | A | O | |
| ATOM | 600 | N | LEU | A | 630 | 7.189 | 14.492 | −3.016 | 1.00 | 48.76 | A | N | |
| ATOM | 601 | CA | LEU | A | 630 | 7.461 | 14.722 | −1.596 | 1.00 | 47.63 | A | C | |
| ATOM | 602 | CB | LEU | A | 630 | 6.162 | 14.567 | −0.795 | 1.00 | 47.73 | A | C | |
| ATOM | 603 | CG | LEU | A | 630 | 6.152 | 14.933 | 0.695 | 1.00 | 47.87 | A | C | |
| ATOM | 604 | CD1 | LEU | A | 630 | 6.488 | 16.413 | 0.908 | 1.00 | 47.17 | A | C | |
| ATOM | 605 | CD2 | LEU | A | 630 | 4.772 | 14.622 | 1.247 | 1.00 | 48.26 | A | C | |
| ATOM | 606 | C | LEU | A | 630 | 8.542 | 13.758 | −1.060 | 1.00 | 46.70 | A | C | |
| ATOM | 607 | O | LEU | A | 630 | 9.429 | 14.152 | −0.294 | 1.00 | 46.04 | A | O | |
| ATOM | 608 | N | MET | A | 631 | 8.466 | 12.492 | −1.454 | 1.00 | 44.91 | A | N | |
| ATOM | 609 | CA | MET | A | 631 | 9.461 | 11.537 | −1.009 | 1.00 | 43.83 | A | C | |
| ATOM | 610 | CB | MET | A | 631 | 9.062 | 10.130 | −1.426 | 1.00 | 44.32 | A | C | |
| ATOM | 611 | CG | MET | A | 631 | 7.891 | 9.569 | −0.656 | 1.00 | 44.22 | A | C | |
| ATOM | 612 | SD | MET | A | 631 | 8.316 | 9.152 | 1.065 | 1.00 | 47.72 | A | S | |
| ATOM | 613 | CE | MET | A | 631 | 10.126 | 9.049 | 0.987 | 1.00 | 45.34 | A | C | |
| ATOM | 614 | C | MET | A | 631 | 10.773 | 11.925 | −1.672 | 1.00 | 43.77 | A | C | |
| ATOM | 615 | O | MET | A | 631 | 11.839 | 11.886 | −1.047 | 1.00 | 44.18 | A | O | |
| ATOM | 616 | N | SER | A | 632 | 10.687 | 12.308 | −2.944 | 1.00 | 43.05 | A | N | |
| ATOM | 617 | CA | SER | A | 632 | 11.866 | 12.711 | −3.697 | 1.00 | 43.11 | A | C | |
| ATOM | 618 | CB | SER | A | 632 | 11.484 | 13.175 | −5.094 | 1.00 | 42.94 | A | C | |
| ATOM | 619 | OG | SER | A | 632 | 10.586 | 12.270 | −5.701 | 1.00 | 47.91 | A | O | |
| ATOM | 620 | C | SER | A | 632 | 12.536 | 13.860 | −2.980 | 1.00 | 42.96 | A | C | |
| ATOM | 621 | O | SER | A | 632 | 13.723 | 14.100 | −3.150 | 1.00 | 44.22 | A | O | |
| ATOM | 622 | N | GLU | A | 633 | 11.766 | 14.582 | −2.183 | 1.00 | 43.52 | A | N | |
| ATOM | 623 | CA | GLU | A | 633 | 12.305 | 15.712 | −1.446 | 1.00 | 44.57 | A | C | |
| ATOM | 624 | CB | GLU | A | 633 | 11.236 | 16.778 | −1.298 | 1.00 | 46.61 | A | C | |
| ATOM | 625 | CG | GLU | A | 633 | 11.672 | 18.020 | −0.570 | 1.00 | 48.46 | A | C | |
| ATOM | 626 | CD | GLU | A | 633 | 10.574 | 19.052 | −0.602 | 1.00 | 49.51 | A | C | |
| ATOM | 627 | OE1 | GLU | A | 633 | 10.189 | 19.436 | −1.732 | 1.00 | 47.74 | A | O | |
| ATOM | 628 | OE2 | GLU | A | 633 | 10.090 | 19.454 | 0.487 | 1.00 | 49.71 | A | O | |
| ATOM | 629 | C | GLU | A | 633 | 12.813 | 15.297 | −0.076 | 1.00 | 44.80 | A | C | |
| ATOM | 630 | O | GLU | A | 633 | 13.878 | 15.746 | 0.352 | 1.00 | 45.44 | A | O | |
| ATOM | 631 | N | LEU | A | 634 | 12.052 | 14.451 | 0.620 | 1.00 | 44.75 | A | N | |
| ATOM | 632 | CA | LEU | A | 634 | 12.486 | 13.990 | 1.936 | 1.00 | 43.68 | A | C | |
| ATOM | 633 | CB | LEU | A | 634 | 11.575 | 12.885 | 2.483 | 1.00 | 42.62 | A | C | |
| ATOM | 634 | CG | LEU | A | 634 | 11.726 | 12.476 | 3.961 | 1.00 | 39.90 | A | C | |
| ATOM | 635 | CD1 | LEU | A | 634 | 11.626 | 10.969 | 4.063 | 1.00 | 40.11 | A | C | |
| ATOM | 636 | CD2 | LEU | A | 634 | 13.041 | 12.933 | 4.533 | 1.00 | 37.97 | A | C | |
| ATOM | 637 | C | LEU | A | 634 | 13.866 | 13.411 | 1.698 | 1.00 | 43.56 | A | C | |
| ATOM | 638 | O | LEU | A | 634 | 14.825 | 13.724 | 2.409 | 1.00 | 44.11 | A | O | |
| ATOM | 639 | N | LYS | A | 635 | 13.963 | 12.572 | 0.675 | 1.00 | 42.93 | A | N | |
| ATOM | 640 | CA | LYS | A | 635 | 15.237 | 11.954 | 0.364 | 1.00 | 43.61 | A | C | |
| ATOM | 641 | CB | LYS | A | 635 | 15.093 | 11.102 | −0.903 | 1.00 | 43.67 | A | C | |
| ATOM | 642 | CG | LYS | A | 635 | 14.249 | 9.851 | −0.646 | 1.00 | 42.81 | A | C | |
| ATOM | 643 | CD | LYS | A | 635 | 13.719 | 9.215 | −1.917 | 1.00 | 44.05 | A | C | |
| ATOM | 644 | CE | LYS | A | 635 | 12.787 | 8.051 | −1.574 | 1.00 | 43.53 | A | C | |
| ATOM | 645 | NZ | LYS | A | 635 | 11.937 | 7.648 | −2.727 | 1.00 | 42.36 | A | N | |
| ATOM | 646 | C | LYS | A | 635 | 16.331 | 13.021 | 0.243 | 1.00 | 42.97 | A | C | |
| ATOM | 647 | O | LYS | A | 635 | 17.283 | 13.044 | 1.043 | 1.00 | 43.13 | A | O | |
| ATOM | 648 | N | ILE | A | 636 | 16.181 | 13.920 | −0.724 | 1.00 | 41.60 | A | N | |
| ATOM | 649 | CA | ILE | A | 636 | 17.151 | 14.986 | −0.916 | 1.00 | 40.52 | A | C | |
| ATOM | 650 | CB | ILE | A | 636 | 16.579 | 16.066 | −1.875 | 1.00 | 40.14 | A | C | |
| ATOM | 651 | CG2 | ILE | A | 636 | 17.075 | 17.435 | −1.486 | 1.00 | 39.55 | A | C | |
| ATOM | 652 | CG1 | ILE | A | 636 | 16.960 | 15.735 | −3.325 | 1.00 | 40.03 | A | C | |
| ATOM | 653 | CD1 | ILE | A | 636 | 16.315 | 14.463 | −3.899 | 1.00 | 39.72 | A | C | |
| ATOM | 654 | C | ILE | A | 636 | 17.495 | 15.589 | 0.451 | 1.00 | 40.21 | A | C | |
| ATOM | 655 | O | ILE | A | 636 | 18.650 | 15.895 | 0.739 | 1.00 | 38.66 | A | O | |
| ATOM | 656 | N | MET | A | 637 | 16.485 | 15.737 | 1.297 | 1.00 | 41.91 | A | N | |
| ATOM | 657 | CA | MET | A | 637 | 16.691 | 16.279 | 2.637 | 1.00 | 43.31 | A | C | |
| ATOM | 658 | CB | MET | A | 637 | 15.353 | 16.479 | 3.353 | 1.00 | 45.76 | A | C | |
| ATOM | 659 | CG | MET | A | 637 | 14.552 | 17.650 | 2.848 | 1.00 | 47.72 | A | C | |
| ATOM | 660 | SD | MET | A | 637 | 15.671 | 19.051 | 2.818 | 1.00 | 54.05 | A | S | |
| ATOM | 661 | CE | MET | A | 637 | 15.347 | 19.836 | 4.443 | 1.00 | 52.49 | A | C | |
| ATOM | 662 | C | MET | A | 637 | 17.572 | 15.382 | 3.496 | 1.00 | 43.08 | A | C | |
| ATOM | 663 | O | MET | A | 637 | 18.513 | 15.856 | 4.130 | 1.00 | 42.20 | A | O | |
| ATOM | 664 | N | SER | A | 638 | 17.271 | 14.087 | 3.518 | 1.00 | 44.00 | A | N | |
| ATOM | 665 | CA | SER | A | 638 | 18.047 | 13.148 | 4.332 | 1.00 | 45.46 | A | C | |
| ATOM | 666 | CB | SER | A | 638 | 17.266 | 11.844 | 4.547 | 1.00 | 45.91 | A | C | |
| ATOM | 667 | OG | SER | A | 638 | 16.909 | 11.235 | 3.320 | 1.00 | 46.46 | A | O | |
| ATOM | 668 | C | SER | A | 638 | 19.422 | 12.828 | 3.765 | 1.00 | 45.50 | A | C | |
| ATOM | 669 | O | SER | A | 638 | 20.197 | 12.109 | 4.396 | 1.00 | 45.66 | A | O | |
| ATOM | 670 | N | HIS | A | 639 | 19.712 | 13.364 | 2.579 | 1.00 | 46.12 | A | N | |
| ATOM | 671 | CA | HIS | A | 639 | 21.002 | 13.166 | 1.900 | 1.00 | 46.30 | A | C | |
| ATOM | 672 | CB | HIS | A | 639 | 20.799 | 13.120 | 0.363 | 1.00 | 48.85 | A | C | |
| ATOM | 673 | CG | HIS | A | 639 | 22.077 | 13.077 | −0.432 | 1.00 | 50.22 | A | C | |
| ATOM | 674 | CD2 | HIS | A | 639 | 22.797 | 14.067 | −1.014 | 1.00 | 50.07 | A | C | |
| ATOM | 675 | ND1 | HIS | A | 639 | 22.769 | 11.907 | −0.681 | 1.00 | 51.40 | A | N | |
| ATOM | 676 | CE1 | HIS | A | 639 | 23.858 | 12.180 | −1.380 | 1.00 | 50.36 | A | C | |
| ATOM | 677 | NE2 | HIS | A | 639 | 23.899 | 13.483 | −1.594 | 1.00 | 49.64 | A | N | |
| ATOM | 678 | C | HIS | A | 639 | 21.926 | 14.333 | 2.255 | 1.00 | 45.06 | A | C | |
| ATOM | 679 | O | HIS | A | 639 | 23.071 | 14.145 | 2.674 | 1.00 | 45.09 | A | O | |
| ATOM | 680 | N | LEU | A | 640 | 21.418 | 15.548 | 2.082 | 1.00 | 43.04 | A | N | |
| ATOM | 681 | CA | LEU | A | 640 | 22.217 | 16.723 | 2.371 | 1.00 | 41.53 | A | C | |
| ATOM | 682 | CB | LEU | A | 640 | 21.428 | 18.003 | 2.055 | 1.00 | 39.83 | A | C | |
| ATOM | 683 | CG | LEU | A | 640 | 20.997 | 18.164 | 0.595 | 1.00 | 37.72 | A | C | |
| ATOM | 684 | CD1 | LEU | A | 640 | 20.139 | 19.389 | 0.473 | 1.00 | 38.42 | A | C | |
| ATOM | 685 | CD2 | LEU | A | 640 | 22.207 | 18.271 | −0.315 | 1.00 | 37.15 | A | C | |
| ATOM | 686 | C | LEU | A | 640 | 22.669 | 16.734 | 3.821 | 1.00 | 40.05 | A | C | |
| ATOM | 687 | O | LEU | A | 640 | 23.778 | 17.157 | 4.123 | 1.00 | 40.86 | A | O | |
| ATOM | 688 | N | GLY | A | 641 | 21.824 | 16.240 | 4.713 | 1.00 | 39.29 | A | N | |
| ATOM | 689 | CA | GLY | A | 641 | 22.171 | 16.260 | 6.119 | 1.00 | 38.12 | A | C | |
| ATOM | 690 | C | GLY | A | 641 | 21.620 | 17.537 | 6.723 | 1.00 | 37.74 | A | C | |
| ATOM | 691 | O | GLY | A | 641 | 20.684 | 18.130 | 6.183 | 1.00 | 38.36 | A | O | |
| ATOM | 692 | N | GLN | A | 642 | 22.201 | 17.987 | 7.824 | 1.00 | 37.63 | A | N | |
| ATOM | 693 | CA | GLN | A | 642 | 21.703 | 19.187 | 8.475 | 1.00 | 38.41 | A | C | |
| ATOM | 694 | CB | GLN | A | 642 | 21.112 | 18.803 | 9.841 | 1.00 | 40.74 | A | C | |
| ATOM | 695 | CG | GLN | A | 642 | 19.899 | 17.862 | 9.727 | 1.00 | 46.32 | A | C | |
| ATOM | 696 | CD | GLN | A | 642 | 19.310 | 17.427 | 11.086 | 1.00 | 50.87 | A | C | |
| ATOM | 697 | OE1 | GLN | A | 642 | 18.988 | 18.258 | 11.951 | 1.00 | 53.07 | A | O | |
| ATOM | 698 | NE2 | GLN | A | 642 | 19.158 | 16.116 | 11.267 | 1.00 | 52.04 | A | N | |
| ATOM | 699 | C | GLN | A | 642 | 22.719 | 20.324 | 8.622 | 1.00 | 36.73 | A | C | |
| ATOM | 700 | O | GLN | A | 642 | 23.887 | 20.100 | 8.921 | 1.00 | 36.06 | A | O | |
| ATOM | 701 | N | HIS | A | 643 | 22.258 | 21.549 | 8.393 | 1.00 | 36.40 | A | N | |
| ATOM | 702 | CA | HIS | A | 643 | 23.100 | 22.737 | 8.513 | 1.00 | 37.09 | A | C | |
| ATOM | 703 | CB | HIS | A | 643 | 23.569 | 23.175 | 7.133 | 1.00 | 36.25 | A | C | |
| ATOM | 704 | CG | HIS | A | 643 | 24.506 | 24.340 | 7.154 | 1.00 | 36.15 | A | C | |
| ATOM | 705 | CD2 | HIS | A | 643 | 25.776 | 24.468 | 6.700 | 1.00 | 34.47 | A | C | |
| ATOM | 706 | ND1 | HIS | A | 643 | 24.139 | 25.585 | 7.624 | 1.00 | 36.77 | A | N | |
| ATOM | 707 | CE1 | HIS | A | 643 | 25.141 | 26.431 | 7.453 | 1.00 | 34.95 | A | C | |
| ATOM | 708 | NE2 | HIS | A | 643 | 26.145 | 25.779 | 6.894 | 1.00 | 34.03 | A | N | |
| ATOM | 709 | C | HIS | A | 643 | 22.335 | 23.880 | 9.211 | 1.00 | 38.26 | A | C | |
| ATOM | 710 | O | HIS | A | 643 | 21.125 | 24.067 | 8.996 | 1.00 | 38.71 | A | O | |
| ATOM | 711 | N | GLU | A | 644 | 23.039 | 24.631 | 10.055 | 1.00 | 37.93 | A | N | |
| ATOM | 712 | CA | GLU | A | 644 | 22.427 | 25.726 | 10.796 | 1.00 | 38.96 | A | C | |
| ATOM | 713 | CB | GLU | A | 644 | 23.499 | 26.504 | 11.573 | 1.00 | 41.54 | A | C | |
| ATOM | 714 | CG | GLU | A | 644 | 24.665 | 26.995 | 10.729 | 1.00 | 48.20 | A | C | |
| ATOM | 715 | CD | GLU | A | 644 | 25.737 | 25.926 | 10.486 | 1.00 | 52.47 | A | C | |
| ATOM | 716 | OE1 | GLU | A | 644 | 25.382 | 24.786 | 10.095 | 1.00 | 54.85 | A | O | |
| ATOM | 717 | OE2 | GLU | A | 644 | 26.944 | 26.232 | 10.669 | 1.00 | 54.22 | A | O | |
| ATOM | 718 | C | GLU | A | 644 | 21.623 | 26.682 | 9.906 | 1.00 | 38.04 | A | C | |
| ATOM | 719 | O | GLU | A | 644 | 20.520 | 27.123 | 10.283 | 1.00 | 36.83 | A | O | |
| ATOM | 720 | N | ASN | A | 645 | 22.165 | 26.963 | 8.718 | 1.00 | 36.67 | A | N | |
| ATOM | 721 | CA | ASN | A | 645 | 21.552 | 27.879 | 7.758 | 1.00 | 34.88 | A | C | |
| ATOM | 722 | CB | ASN | A | 645 | 22.671 | 28.652 | 7.072 | 1.00 | 34.55 | A | C | |
| ATOM | 723 | CG | ASN | A | 645 | 23.458 | 29.487 | 8.061 | 1.00 | 36.13 | A | C | |
| ATOM | 724 | OD1 | ASN | A | 645 | 24.636 | 29.820 | 7.848 | 1.00 | 35.85 | A | O | |
| ATOM | 725 | ND2 | ASN | A | 645 | 22.799 | 29.842 | 9.165 | 1.00 | 36.31 | A | N | |
| ATOM | 726 | C | ASN | A | 645 | 20.603 | 27.261 | 6.729 | 1.00 | 35.00 | A | C | |
| ATOM | 727 | O | ASN | A | 645 | 20.280 | 27.878 | 5.706 | 1.00 | 35.18 | A | O | |
| ATOM | 728 | N | ILE | A | 646 | 20.139 | 26.047 | 7.010 | 1.00 | 34.34 | A | N | |
| ATOM | 729 | CA | ILE | A | 646 | 19.215 | 25.338 | 6.126 | 1.00 | 33.01 | A | C | |
| ATOM | 730 | CB | ILE | A | 646 | 19.885 | 24.064 | 5.565 | 1.00 | 34.95 | A | C | |
| ATOM | 731 | CG2 | ILE | A | 646 | 18.873 | 23.131 | 4.965 | 1.00 | 36.91 | A | C | |
| ATOM | 732 | CG1 | ILE | A | 646 | 20.905 | 24.458 | 4.510 | 1.00 | 38.74 | A | C | |
| ATOM | 733 | CD1 | ILE | A | 646 | 20.311 | 25.268 | 3.379 | 1.00 | 41.12 | A | C | |
| ATOM | 734 | C | ILE | A | 646 | 18.020 | 24.950 | 6.980 | 1.00 | 32.12 | A | C | |
| ATOM | 735 | O | ILE | A | 646 | 18.155 | 24.819 | 8.200 | 1.00 | 32.51 | A | O | |
| ATOM | 736 | N | VAL | A | 647 | 16.846 | 24.800 | 6.376 | 1.00 | 30.96 | A | N | |
| ATOM | 737 | CA | VAL | A | 647 | 15.706 | 24.385 | 7.171 | 1.00 | 29.78 | A | C | |
| ATOM | 738 | CB | VAL | A | 647 | 14.352 | 24.885 | 6.601 | 1.00 | 29.12 | A | C | |
| ATOM | 739 | CG1 | VAL | A | 647 | 13.202 | 24.336 | 7.436 | 1.00 | 28.08 | A | C | |
| ATOM | 740 | CG2 | VAL | A | 647 | 14.294 | 26.413 | 6.648 | 1.00 | 28.12 | A | C | |
| ATOM | 741 | C | VAL | A | 647 | 15.771 | 22.869 | 7.154 | 1.00 | 30.17 | A | C | |
| ATOM | 742 | O | VAL | A | 647 | 15.101 | 22.196 | 6.376 | 1.00 | 30.94 | A | O | |
| ATOM | 743 | N | ASN | A | 648 | 16.616 | 22.350 | 8.032 | 1.00 | 29.94 | A | N | |
| ATOM | 744 | CA | ASN | A | 648 | 16.844 | 20.927 | 8.190 | 1.00 | 30.90 | A | C | |
| ATOM | 745 | CB | ASN | A | 648 | 17.723 | 20.714 | 9.416 | 1.00 | 30.96 | A | C | |
| ATOM | 746 | CG | ASN | A | 648 | 19.010 | 21.503 | 9.348 | 1.00 | 29.79 | A | C | |
| ATOM | 747 | OD1 | ASN | A | 648 | 19.876 | 21.236 | 8.510 | 1.00 | 29.29 | A | O | |
| ATOM | 748 | ND2 | ASN | A | 648 | 19.142 | 22.488 | 10.226 | 1.00 | 28.64 | A | N | |
| ATOM | 749 | C | ASN | A | 648 | 15.612 | 20.026 | 8.335 | 1.00 | 31.70 | A | C | |
| ATOM | 750 | O | ASN | A | 648 | 14.514 | 20.474 | 8.662 | 1.00 | 30.96 | A | O | |
| ATOM | 751 | N | LEU | A | 649 | 15.826 | 18.738 | 8.084 | 1.00 | 32.54 | A | N | |
| ATOM | 752 | CA | LEU | A | 649 | 14.786 | 17.730 | 8.242 | 1.00 | 33.14 | A | C | |
| ATOM | 753 | CB | LEU | A | 649 | 15.089 | 16.528 | 7.344 | 1.00 | 28.99 | A | C | |
| ATOM | 754 | CG | LEU | A | 649 | 14.287 | 15.233 | 7.485 | 1.00 | 26.96 | A | C | |
| ATOM | 755 | CD1 | LEU | A | 649 | 12.848 | 15.392 | 7.009 | 1.00 | 23.89 | A | C | |
| ATOM | 756 | CD2 | LEU | A | 649 | 14.970 | 14.190 | 6.649 | 1.00 | 26.32 | A | C | |
| ATOM | 757 | C | LEU | A | 649 | 14.924 | 17.327 | 9.721 | 1.00 | 36.01 | A | C | |
| ATOM | 758 | O | LEU | A | 649 | 15.926 | 17.680 | 10.373 | 1.00 | 38.12 | A | O | |
| ATOM | 759 | N | LEU | A | 650 | 13.937 | 16.614 | 10.258 | 1.00 | 36.80 | A | N | |
| ATOM | 760 | CA | LEU | A | 650 | 13.995 | 16.173 | 11.647 | 1.00 | 37.86 | A | C | |
| ATOM | 761 | CB | LEU | A | 650 | 13.354 | 17.211 | 12.579 | 1.00 | 36.61 | A | C | |
| ATOM | 762 | CG | LEU | A | 650 | 14.113 | 18.538 | 12.793 | 1.00 | 36.51 | A | C | |
| ATOM | 763 | CD1 | LEU | A | 650 | 13.215 | 19.583 | 13.461 | 1.00 | 35.25 | A | C | |
| ATOM | 764 | CD2 | LEU | A | 650 | 15.363 | 18.295 | 13.627 | 1.00 | 36.71 | A | C | |
| ATOM | 765 | C | LEU | A | 650 | 13.274 | 14.836 | 11.766 | 1.00 | 40.29 | A | C | |
| ATOM | 766 | O | LEU | A | 650 | 13.545 | 14.048 | 12.689 | 1.00 | 41.90 | A | O | |
| ATOM | 767 | N | GLY | A | 651 | 12.367 | 14.581 | 10.820 | 1.00 | 41.26 | A | N | |
| ATOM | 768 | CA | GLY | A | 651 | 11.611 | 13.337 | 10.822 | 1.00 | 42.25 | A | C | |
| ATOM | 769 | C | GLY | A | 651 | 10.513 | 13.251 | 9.769 | 1.00 | 42.59 | A | C | |
| ATOM | 770 | O | GLY | A | 651 | 10.261 | 14.207 | 9.023 | 1.00 | 42.13 | A | O | |
| ATOM | 771 | N | ALA | A | 652 | 9.861 | 12.093 | 9.711 | 1.00 | 42.68 | A | N | |
| ATOM | 772 | CA | ALA | A | 652 | 8.788 | 11.861 | 8.760 | 1.00 | 43.88 | A | C | |
| ATOM | 773 | CB | ALA | A | 652 | 9.368 | 11.465 | 7.418 | 1.00 | 42.81 | A | C | |
| ATOM | 774 | C | ALA | A | 652 | 7.830 | 10.781 | 9.260 | 1.00 | 45.91 | A | C | |
| ATOM | 775 | O | ALA | A | 652 | 8.134 | 10.048 | 10.201 | 1.00 | 46.00 | A | O | |
| ATOM | 776 | N | CYS | A | 653 | 6.673 | 10.678 | 8.616 | 1.00 | 49.02 | A | N | |
| ATOM | 777 | CA | CYS | A | 653 | 5.669 | 9.691 | 9.002 | 1.00 | 52.05 | A | C | |
| ATOM | 778 | CB | CYS | A | 653 | 4.424 | 10.397 | 9.554 | 1.00 | 50.34 | A | C | |
| ATOM | 779 | SG | CYS | A | 653 | 4.711 | 11.301 | 11.087 | 1.00 | 48.96 | A | S | |
| ATOM | 780 | C | CYS | A | 653 | 5.251 | 8.797 | 7.838 | 1.00 | 55.38 | A | C | |
| ATOM | 781 | O | CYS | A | 653 | 4.067 | 8.494 | 7.690 | 1.00 | 57.20 | A | O | |
| ATOM | 782 | N | THR | A | 654 | 6.209 | 8.364 | 7.020 | 1.00 | 58.03 | A | N | |
| ATOM | 783 | CA | THR | A | 654 | 5.896 | 7.519 | 5.864 | 1.00 | 59.93 | A | C | |
| ATOM | 784 | CB | THR | A | 654 | 7.178 | 7.075 | 5.129 | 1.00 | 60.45 | A | C | |
| ATOM | 785 | OG1 | THR | A | 654 | 8.180 | 6.683 | 6.084 | 1.00 | 59.85 | A | O | |
| ATOM | 786 | CG2 | THR | A | 654 | 7.690 | 8.209 | 4.239 | 1.00 | 61.12 | A | C | |
| ATOM | 787 | C | THR | A | 654 | 5.061 | 6.266 | 6.127 | 1.00 | 61.61 | A | C | |
| ATOM | 788 | O | THR | A | 654 | 4.550 | 5.661 | 5.180 | 1.00 | 62.11 | A | O | |
| ATOM | 789 | N | HIS | A | 655 | 4.920 | 5.873 | 7.394 | 1.00 | 63.46 | A | N | |
| ATOM | 790 | CA | HIS | A | 655 | 4.146 | 4.675 | 7.739 | 1.00 | 64.35 | A | C | |
| ATOM | 791 | CB | HIS | A | 655 | 5.075 | 3.566 | 8.260 | 1.00 | 64.71 | A | C | |
| ATOM | 792 | CG | HIS | A | 655 | 6.018 | 3.042 | 7.222 | 1.00 | 66.29 | A | C | |
| ATOM | 793 | CD2 | HIS | A | 655 | 7.371 | 3.047 | 7.164 | 1.00 | 66.69 | A | C | |
| ATOM | 794 | ND1 | HIS | A | 655 | 5.584 | 2.505 | 6.027 | 1.00 | 65.94 | A | N | |
| ATOM | 795 | CE1 | HIS | A | 655 | 6.629 | 2.211 | 5.274 | 1.00 | 66.66 | A | C | |
| ATOM | 796 | NE2 | HIS | A | 655 | 7.726 | 2.531 | 5.940 | 1.00 | 67.30 | A | N | |
| ATOM | 797 | C | HIS | A | 655 | 3.040 | 4.920 | 8.750 | 1.00 | 64.14 | A | C | |
| ATOM | 798 | O | HIS | A | 655 | 3.229 | 5.629 | 9.740 | 1.00 | 64.66 | A | O | |
| ATOM | 799 | N | GLY | A | 656 | 1.885 | 4.316 | 8.500 | 1.00 | 63.68 | A | N | |
| ATOM | 800 | CA | GLY | A | 656 | 0.770 | 4.480 | 9.408 | 1.00 | 63.01 | A | C | |
| ATOM | 801 | C | GLY | A | 656 | 0.210 | 5.504 | 8.878 | 1.00 | 62.94 | A | C | |
| ATOM | 802 | O | GLY | A | 656 | −1.090 | 5.973 | 9.611 | 1.00 | 63.90 | A | O | |
| ATOM | 803 | N | GLY | A | 657 | −0.059 | 5.857 | 7.604 | 1.00 | 61.27 | A | N | |
| ATOM | 804 | CA | GLY | A | 657 | −0.962 | 6.822 | 7.008 | 1.00 | 60.06 | A | C | |
| ATOM | 805 | C | GLY | A | 657 | 0.314 | 7.704 | 5.961 | 1.00 | 59.32 | A | C | |
| ATOM | 806 | O | GLY | A | 657 | 0.507 | 7.242 | 5.168 | 1.00 | 60.41 | A | O | |
| ATOM | 807 | N | PRO | A | 658 | 0.668 | 8.993 | 5.932 | 1.00 | 58.00 | A | N | |
| ATOM | 808 | CD | PRO | A | 658 | −1.741 | 9.633 | 6.711 | 1.00 | 58.15 | A | C | |
| ATOM | 809 | CA | PRO | A | 658 | −0.100 | 9.927 | 4.956 | 1.00 | 56.17 | A | C | |
| ATOM | 810 | CB | PRO | A | 658 | 1.029 | 11.142 | 5.046 | 1.00 | 57.98 | A | C | |
| ATOM | 811 | CG | PRO | A | 658 | −2.297 | 10.604 | 5.710 | 1.00 | 58.85 | A | C | |
| ATOM | 812 | C | PRO | A | 658 | 1.332 | 10.301 | 5.316 | 1.00 | 54.33 | A | C | |
| ATOM | 813 | O | PRO | A | 658 | 1.698 | 10.304 | 6.493 | 1.00 | 53.99 | A | O | |
| ATOM | 814 | N | VAL | A | 659 | 2.136 | 10.613 | 4.303 | 1.00 | 51.99 | A | N | |
| ATOM | 815 | CA | VAL | A | 659 | 3.516 | 11.018 | 4.529 | 1.00 | 48.73 | A | C | |
| ATOM | 816 | CB | VAL | A | 659 | 4.334 | 11.016 | 3.219 | 1.00 | 49.60 | A | C | |
| ATOM | 817 | CG1 | VAL | A | 659 | 5.619 | 11.831 | 3.398 | 1.00 | 49.75 | A | C | |
| ATOM | 818 | CG2 | VAL | A | 659 | 4.677 | 9.587 | 2.823 | 1.00 | 50.37 | A | C | |
| ATOM | 819 | C | VAL | A | 659 | 3.504 | 12.434 | 5.086 | 1.00 | 46.09 | A | C | |
| ATOM | 820 | O | VAL | A | 659 | 2.824 | 13.306 | 4.556 | 1.00 | 45.70 | A | O | |
| ATOM | 821 | N | LEU | A | 660 | 4.260 | 12.648 | 6.157 | 1.00 | 43.65 | A | N | |
| ATOM | 822 | CA | LEU | A | 660 | 4.359 | 13.951 | 6.804 | 1.00 | 40.46 | A | C | |
| ATOM | 823 | CB | LEU | A | 660 | 3.611 | 13.946 | 8.133 | 1.00 | 38.99 | A | C | |
| ATOM | 824 | CG | LEU | A | 660 | 2.199 | 13.363 | 8.182 | 1.00 | 37.65 | A | C | |
| ATOM | 825 | CD1 | LEU | A | 660 | 1.760 | 13.331 | 9.640 | 1.00 | 36.70 | A | C | |
| ATOM | 826 | CD2 | LEU | A | 660 | 1.229 | 14.185 | 7.325 | 1.00 | 35.95 | A | C | |
| ATOM | 827 | C | LEU | A | 660 | 5.834 | 14.196 | 7.074 | 1.00 | 39.69 | A | C | |
| ATOM | 828 | O | LEU | A | 660 | 6.457 | 13.485 | 7.864 | 1.00 | 38.69 | A | O | |
| ATOM | 829 | N | VAL | A | 661 | 6.384 | 15.203 | 6.410 | 1.00 | 39.08 | A | N | |
| ATOM | 830 | CA | VAL | A | 661 | 7.786 | 15.565 | 6.547 | 1.00 | 37.28 | A | C | |
| ATOM | 831 | CB | VAL | A | 661 | 8.269 | 16.165 | 5.203 | 1.00 | 36.94 | A | C | |
| ATOM | 832 | CG1 | VAL | A | 661 | 9.759 | 16.430 | 5.236 | 1.00 | 37.84 | A | C | |
| ATOM | 833 | CG2 | VAL | A | 661 | 7.919 | 15.207 | 4.072 | 1.00 | 34.93 | A | C | |
| ATOM | 834 | C | VAL | A | 661 | 7.920 | 16.569 | 7.715 | 1.00 | 37.43 | A | C | |
| ATOM | 835 | O | VAL | A | 661 | 7.359 | 17.675 | 7.681 | 1.00 | 38.10 | A | O | |
| ATOM | 836 | N | ILE | A | 662 | 8.649 | 16.168 | 8.755 | 1.00 | 36.23 | A | N | |
| ATOM | 837 | CA | ILE | A | 662 | 8.831 | 16.997 | 9.953 | 1.00 | 33.62 | A | C | |
| ATOM | 838 | GB | ILE | A | 662 | 9.000 | 16.121 | 11.200 | 1.00 | 32.67 | A | C | |
| ATOM | 839 | CG2 | ILE | A | 662 | 8.828 | 16.971 | 12.458 | 1.00 | 32.70 | A | C | |
| ATOM | 840 | CG1 | ILE | A | 662 | 7.986 | 14.973 | 11.159 | 1.00 | 33.80 | A | C | |
| ATOM | 841 | CD1 | ILE | A | 662 | 8.087 | 13.984 | 12.307 | 1.00 | 32.32 | A | C | |
| ATOM | 842 | C | ILE | A | 662 | 10.064 | 17.871 | 9.861 | 1.00 | 33.11 | A | C | |
| ATOM | 843 | O | ILE | A | 662 | 11.183 | 17.375 | 10.008 | 1.00 | 34.70 | A | O | |
| ATOM | 844 | N | THR | A | 663 | 9.883 | 19.166 | 9.639 | 1.00 | 31.83 | A | N | |
| ATOM | 845 | CA | THR | A | 663 | 11.044 | 20.043 | 9.543 | 1.00 | 30.37 | A | C | |
| ATOM | 846 | CG | THR | A | 663 | 11.148 | 20.705 | 8.134 | 1.00 | 28.46 | A | C | |
| ATOM | 847 | OG1 | THR | A | 663 | 10.074 | 21.623 | 7.929 | 1.00 | 23.93 | A | O | |
| ATOM | 848 | CG2 | THR | A | 663 | 11.084 | 19.634 | 7.054 | 1.00 | 28.18 | A | C | |
| ATOM | 849 | C | THR | A | 663 | 11.084 | 21.109 | 10.626 | 1.00 | 31.37 | A | C | |
| ATOM | 850 | O | THR | A | 663 | 10.118 | 21.291 | 11.387 | 1.00 | 31.98 | A | O | |
| ATOM | 851 | N | GLU | A | 664 | 12.214 | 21.804 | 10.697 | 1.00 | 31.85 | A | N | |
| ATOM | 852 | CA | GLU | A | 664 | 12.420 | 22.847 | 11.695 | 1.00 | 32.96 | A | C | |
| ATOM | 853 | CB | GLU | A | 664 | 13.766 | 23.552 | 11.470 | 1.00 | 31.62 | A | C | |
| ATOM | 854 | CG | GLU | A | 664 | 14.974 | 22.696 | 11.797 | 1.00 | 30.91 | A | C | |
| ATOM | 855 | CD | GLU | A | 664 | 16.276 | 23.325 | 11.334 | 1.00 | 30.03 | A | C | |
| ATOM | 856 | OE1 | GLU | A | 664 | 16.344 | 23.775 | 10.164 | 1.00 | 27.70 | A | O | |
| ATOM | 857 | OE2 | GLU | A | 664 | 17.231 | 23.356 | 12.143 | 1.00 | 29.73 | A | O | |
| ATOM | 858 | C | GLU | A | 664 | 11.319 | 23.885 | 11.683 | 1.00 | 33.49 | A | C | |
| ATOM | 859 | O | GLU | A | 664 | 10.645 | 24.077 | 10.673 | 1.00 | 33.09 | A | O | |
| ATOM | 860 | N | TYR | A | 665 | 11.141 | 24.548 | 12.820 | 1.00 | 35.24 | A | N | |
| ATOM | 861 | CA | TYR | A | 665 | 10.146 | 25.600 | 12.921 | 1.00 | 37.77 | A | C | |
| ATOM | 862 | CB | TYR | A | 665 | 9.031 | 25.214 | 13.897 | 1.00 | 36.57 | A | C | |
| ATOM | 863 | CG | TYR | A | 665 | 8.170 | 26.401 | 14.253 | 1.00 | 35.04 | A | C | |
| ATOM | 864 | CD1 | TYR | A | 665 | 7.151 | 26.837 | 13.403 | 1.00 | 34.36 | A | C | |
| ATOM | 865 | CE1 | TYR | A | 665 | 6.422 | 27.996 | 13.692 | 1.00 | 34.05 | A | C | |
| ATOM | 866 | CD2 | TYR | A | 665 | 8.436 | 27.147 | 15.402 | 1.00 | 34.11 | A | C | |
| ATOM | 867 | CE2 | TYR | A | 665 | 7.723 | 28.296 | 15.697 | 1.00 | 34.50 | A | C | |
| ATOM | 868 | CZ | TYR | A | 665 | 6.719 | 28.718 | 14.845 | 1.00 | 34.33 | A | C | |
| ATOM | 869 | OH | TYR | A | 665 | 6.021 | 29.859 | 15.176 | 1.00 | 35.67 | A | O | |
| ATOM | 870 | C | TYR | A | 665 | 10.789 | 26.913 | 13.387 | 1.00 | 39.64 | A | C | |
| ATOM | 871 | O | TYR | A | 665 | 11.057 | 27.088 | 14.579 | 1.00 | 39.44 | A | O | |
| ATOM | 872 | N | CYS | A | 666 | 11.033 | 27.827 | 12.444 | 1.00 | 40.96 | A | N | |
| ATOM | 873 | CA | CYS | A | 666 | 11.616 | 29.135 | 12.758 | 1.00 | 41.83 | A | C | |
| ATOM | 874 | CB | CYS | A | 666 | 12.280 | 29.726 | 11.510 | 1.00 | 43.59 | A | C | |
| ATOM | 875 | SG | CYS | A | 666 | 13.353 | 28.545 | 10.653 | 1.00 | 48.95 | A | S | |
| ATOM | 876 | C | CYS | A | 666 | 10.477 | 30.045 | 13.243 | 1.00 | 40.90 | A | C | |
| ATOM | 877 | O | CYS | A | 666 | 9.463 | 30.215 | 12.551 | 1.00 | 40.75 | A | O | |
| ATOM | 878 | N | CYS | A | 667 | 10.659 | 30.637 | 14.424 | 1.00 | 39.87 | A | N | |
| ATOM | 879 | CA | CYS | A | 667 | 9.638 | 31.475 | 15.047 | 1.00 | 38.75 | A | C | |
| ATOM | 880 | CB | CYS | A | 667 | 9.884 | 31.535 | 16.544 | 1.00 | 37.04 | A | C | |
| ATOM | 881 | SG | CYS | A | 667 | 11.303 | 32.554 | 16.930 | 1.00 | 37.20 | A | S | |
| ATOM | 882 | C | CYS | A | 667 | 9.438 | 32.905 | 14.547 | 1.00 | 38.99 | A | C | |
| ATOM | 883 | O | CYS | A | 667 | 8.480 | 33.560 | 14.966 | 1.00 | 39.50 | A | O | |
| ATOM | 884 | N | TYR | A | 668 | 10.301 | 33.406 | 13.666 | 1.00 | 38.76 | A | N | |
| ATOM | 885 | CA | TYR | A | 668 | 10.118 | 34.778 | 13.192 | 1.00 | 37.86 | A | C | |
| ATOM | 886 | CB | TYR | A | 668 | 11.423 | 35.567 | 13.289 | 1.00 | 39.08 | A | C | |
| ATOM | 887 | CG | TYR | A | 668 | 11.950 | 35.704 | 14.700 | 1.00 | 40.51 | A | C | |
| ATOM | 888 | CD1 | TYR | A | 668 | 13.154 | 35.108 | 15.067 | 1.00 | 40.65 | A | C | |
| ATOM | 889 | CE1 | TYR | A | 668 | 13.631 | 35.194 | 16.367 | 1.00 | 41.44 | A | C | |
| ATOM | 890 | CD2 | TYR | A | 668 | 11.229 | 36.401 | 15.677 | 1.00 | 40.42 | A | C | |
| ATOM | 891 | CE2 | TYR | A | 668 | 11.698 | 36.490 | 16.987 | 1.00 | 40.93 | A | C | |
| ATOM | 892 | CZ | TYR | A | 668 | 12.901 | 35.882 | 17.322 | 1.00 | 41.28 | A | C | |
| ATOM | 893 | OH | TYR | A | 668 | 13.387 | 35.942 | 18.608 | 1.00 | 42.08 | A | O | |
| ATOM | 894 | C | TYR | A | 668 | 9.567 | 34.921 | 11.787 | 1.00 | 37.66 | A | C | |
| ATOM | 895 | O | TYR | A | 668 | 9.465 | 36.038 | 11.275 | 1.00 | 38.42 | A | O | |
| ATOM | 896 | N | GLY | A | 669 | 9.215 | 33.811 | 11.146 | 1.00 | 36.39 | A | N | |
| ATOM | 897 | CA | GLY | A | 669 | 8.664 | 33.917 | 9.800 | 1.00 | 35.01 | A | C | |
| ATOM | 898 | C | GLY | A | 669 | 9.665 | 34.229 | 8.698 | 1.00 | 33.23 | A | C | |
| ATOM | 899 | O | GLY | A | 669 | 10.865 | 34.410 | 8.962 | 1.00 | 31.61 | A | 0 | |
| ATOM | 900 | N | ASP | A | 670 | 9.185 | 34.301 | 7.458 | 1.00 | 32.96 | A | N | |
| ATOM | 901 | CA | ASP | A | 670 | 10.097 | 34.567 | 6.351 | 1.00 | 34.51 | A | C | |
| ATOM | 902 | CB | ASP | A | 670 | 9.425 | 34.356 | 5.004 | 1.00 | 36.89 | A | C | |
| ATOM | 903 | CG | ASP | A | 670 | 8.277 | 35.291 | 4.786 | 1.00 | 39.07 | A | C | |
| ATOM | 904 | OD1 | ASP | A | 670 | 7.331 | 35.226 | 5.597 | 1.00 | 42.09 | A | 0 | |
| ATOM | 905 | OD2 | ASP | A | 670 | 8.317 | 36.078 | 3.814 | 1.00 | 38.90 | A | O | |
| ATOM | 906 | C | ASP | A | 670 | 10.721 | 35.948 | 6.376 | 1.00 | 33.50 | A | C | |
| ATOM | 907 | O | ASP | A | 670 | 10.141 | 36.917 | 6.871 | 1.00 | 34.11 | A | O | |
| ATOM | 908 | N | LEU | A | 671 | 11.926 | 36.007 | 5.828 | 1.00 | 32.00 | A | N | |
| ATOM | 909 | CA | LEU | A | 671 | 12.717 | 37.220 | 5.766 | 1.00 | 30.56 | A | C | |
| ATOM | 910 | CB | LEU | A | 671 | 14.049 | 36.921 | 5.086 | 1.00 | 29.38 | A | C | |
| ATOM | 911 | CG | LEU | A | 671 | 15.024 | 38.079 | 5.060 | 1.00 | 27.72 | A | C | |
| ATOM | 912 | CD1 | LEU | A | 671 | 15.297 | 38.580 | 6.476 | 1.00 | 28.85 | A | C | |
| ATOM | 913 | CD2 | LEU | A | 671 | 16.343 | 37.634 | 4.468 | 1.00 | 25.25 | A | C | |
| ATOM | 914 | C | LEU | A | 671 | 12.047 | 38.380 | 5.055 | 1.00 | 30.73 | A | C | |
| ATOM | 915 | O | LEU | A | 671 | 12.006 | 39.484 | 5.597 | 1.00 | 31.35 | A | O | |
| ATOM | 916 | N | LEU | A | 672 | 11.524 | 38.134 | 3.847 | 1.00 | 31.36 | A | N | |
| ATOM | 917 | CA | LEU | A | 672 | 10.876 | 39.191 | 3.054 | 1.00 | 29.11 | A | C | |
| ATOM | 918 | CB | LEU | A | 672 | 10.079 | 38.609 | 1.875 | 1.00 | 27.63 | A | C | |
| ATOM | 919 | CG | LEU | A | 672 | 9.647 | 39.776 | 0.976 | 1.00 | 29.12 | A | C | |
| ATOM | 920 | CD1 | LEU | A | 672 | 10.920 | 40.310 | 0.321 | 1.00 | 27.18 | A | C | |
| ATOM | 921 | CD2 | LEU | A | 672 | 8.611 | 39.391 | −0.088 | 1.00 | 25.81 | A | C | |
| ATOM | 922 | C | LEU | A | 672 | 9.947 | 39.997 | 3.949 | 1.00 | 28.12 | A | C | |
| ATOM | 923 | O | LEU | A | 672 | 10.204 | 41.162 | 4.234 | 1.00 | 27.96 | A | O | |
| ATOM | 924 | N | ASN | A | 673 | 8.872 | 39.374 | 4.410 | 1.00 | 28.50 | A | N | |
| ATOM | 925 | CA | ASN | A | 673 | 7.958 | 40.081 | 5.284 | 1.00 | 30.72 | A | C | |
| ATOM | 926 | CB | ASN | A | 673 | 6.897 | 39.126 | 5.806 | 1.00 | 31.32 | A | C | |
| ATOM | 927 | CG | ASN | A | 673 | 5.968 | 38.656 | 4.690 | 1.00 | 37.25 | A | C | |
| ATOM | 928 | OD1 | ASN | A | 673 | 5.356 | 39.484 | 3.976 | 1.00 | 38.63 | A | O | |
| ATOM | 929 | ND2 | ASN | A | 673 | 5.866 | 37.329 | 4.510 | 1.00 | 37.97 | A | N | |
| ATOM | 930 | C | ASN | A | 673 | 8.703 | 40.779 | 6.419 | 1.00 | 31.06 | A | C | |
| ATOM | 931 | O | ASN | A | 673 | 8.523 | 41.978 | 6.641 | 1.00 | 32.69 | A | O | |
| ATOM | 932 | N | PHE | A | 674 | 9.569 | 40.060 | 7.116 | 1.00 | 30.27 | A | N | |
| ATOM | 933 | CA | PHE | A | 674 | 10.316 | 40.675 | 8.200 | 1.00 | 30.42 | A | C | |
| ATOM | 934 | CB | PHE | A | 674 | 11.417 | 39.734 | 8.653 | 1.00 | 28.99 | A | C | |
| ATOM | 935 | CG | PHE | A | 674 | 12.209 | 40.252 | 9.805 | 1.00 | 27.40 | A | C | |
| ATOM | 936 | CD1 | PHE | A | 674 | 11.751 | 40.083 | 11.107 | 1.00 | 26.16 | A | C | |
| ATOM | 937 | CD2 | PHE | A | 674 | 13.432 | 40.884 | 9.592 | 1.00 | 27.49 | A | C | |
| ATOM | 938 | CE1 | PHE | A | 674 | 12.510 | 40.530 | 12.194 | 1.00 | 26.70 | A | C | |
| ATOM | 939 | CE2 | PHE | A | 674 | 14.201 | 41.336 | 10.672 | 1.00 | 27.78 | A | C | |
| ATOM | 940 | CZ | PHE | A | 674 | 13.735 | 41.153 | 11.979 | 1.00 | 26.07 | A | C | |
| ATOM | 941 | C | PHE | A | 674 | 10.933 | 42.016 | 7.761 | 1.00 | 32.01 | A | C | |
| ATOM | 942 | O | PHE | A | 674 | 10.614 | 43.079 | 8.312 | 1.00 | 31.59 | A | O | |
| ATOM | 943 | N | LEU | A | 675 | 11.828 | 41.957 | 6.777 | 1.00 | 33.12 | A | N | |
| ATOM | 944 | CA | LEU | A | 675 | 12.463 | 43.154 | 6.254 | 1.00 | 33.67 | A | C | |
| ATOM | 945 | CB | LEU | A | 675 | 13.081 | 42.901 | 4.883 | 1.00 | 32.87 | A | C | |
| ATOM | 946 | CG | LEU | A | 675 | 14.475 | 42.270 | 4.839 | 1.00 | 32.88 | A | C | |
| ATOM | 947 | CD1 | LEU | A | 675 | 14.937 | 42.245 | 3.382 | 1.00 | 31.65 | A | C | |
| ATOM | 948 | CD2 | LEU | A | 675 | 15.462 | 43.075 | 5.709 | 1.00 | 3.0.77 | A | C | |
| ATOM | 949 | C | LEU | A | 675 | 11.418 | 44.225 | 6.107 | 1.00 | 35.58 | A | C | |
| ATOM | 950 | O | LEU | A | 675 | 11.634 | 45.366 | 6.500 | 1.00 | 37.69 | A | O | |
| ATOM | 951 | N | ARG | A | 676 | 10.272 | 43.868 | 5.546 | 1.00 | 37.06 | A | N | |
| ATOM | 952 | CA | ARG | A | 676 | 9.223 | 44.856 | 5.365 | 1.00 | 39.79 | A | C | |
| ATOM | 953 | CB | ARG | A | 676 | 8.083 | 44.235 | 4.575 | 1.00 | 37.41 | A | C | |
| ATOM | 954 | CG | ARG | A | 676 | 8.495 | 44.028 | 3.167 | 1.00 | 36.66 | A | C | |
| ATOM | 955 | CD | ARG | A | 676 | 7.495 | 43.242 | 2.382 | 1.00 | 37.47 | A | C | |
| ATOM | 956 | NE | ARG | A | 676 | 7.818 | 43.340 | 0.966 | 1.00 | 37.69 | A | N | |
| ATOM | 957 | CZ | ARG | A | 676 | 7.211 | 42.657 | 0.007 | 1.00 | 38.35 | A | C | |
| ATOM | 958 | NH1 | ARG | A | 676 | 6.236 | 41.801 | 0.307 | 1.00 | 36.82 | A | N | |
| ATOM | 959 | NH2 | ARG | A | 676 | 7.569 | 42.861 | −4.257 | 1.00 | 38.52 | A | N | |
| ATOM | 960 | C | ARG | A | 676 | 8.705 | 45.515 | 6.656 | 1.00 | 43.19 | A | C | |
| ATOM | 961 | O | ARG | A | 676 | 8.761 | 46.746 | 6.787 | 1.00 | 44.01 | A | O | |
| ATOM | 962 | N | ARG | A | 677 | 8.225 | 44.719 | 7.612 | 1.00 | 44.82 | A | N | |
| ATOM | 963 | CA | ARG | A | 677 | 7.716 | 45.293 | 8.849 | 1.00 | 47.84 | A | C | |
| ATOM | 964 | CB | ARG | A | 677 | 7.126 | 44.200 | 9.750 | 1.00 | 47.26 | A | C | |
| ATOM | 965 | CG | ARG | A | 677 | 8.142 | 43.508 | 10.632 | 1.00 | 46.40 | A | C | |
| ATOM | 966 | CD | ARG | A | 677 | 7.461 | 42.657 | 11.687 | 1.00 | 47.38 | A | C | |
| ATOM | 967 | NE | ARG | A | 677 | 8.419 | 42.187 | 12.685 | 1.00 | 47.37 | A | N | |
| ATOM | 968 | CZ | ARG | A | 677 | 8.083 | 41.617 | 13.839 | 1.00 | 47.14 | A | C | |
| ATOM | 969 | NH1 | ARG | A | 677 | 6.800 | 41.440 | 14.153 | 1.00 | 45.13 | A | N | |
| ATOM | 970 | NH2 | ARG | A | 677 | 9.036 | 41.266 | 14.682 | 1.00 | 48.51 | A | N | |
| ATOM | 971 | C | ARG | A | 677 | 8.766 | 46.105 | 9.641 | 1.00 | 50.32 | A | C | |
| ATOM | 972 | O | ARG | A | 677 | 8.413 | 47.073 | 10.324 | 1.00 | 52.40 | A | O | |
| ATOM | 973 | N | LYS | A | 678 | 10.043 | 45.727 | 9.549 | 1.00 | 51.64 | A | N | |
| ATOM | 974 | CA | LYS | A | 678 | 11.113 | 46.427 | 10.282 | 1.00 | 52.12 | A | C | |
| ATOM | 975 | CB | LYS | A | 678 | 12.089 | 45.407 | 10.912 | 1.00 | 52.78 | A | C | |
| ATOM | 976 | CG | LYS | A | 678 | 11.594 | 44.627 | 12.153 | 1.00 | 54.58 | A | C | |
| ATOM | 977 | CD | LYS | A | 678 | 11.663 | 45.467 | 13.448 | 1.00 | 56.99 | A | C | |
| ATOM | 978 | CE | LYS | A | 678 | 11.827 | 44.610 | 14.726 | 1.00 | 57.75 | A | C | |
| ATOM | 979 | NZ | LYS | A | 678 | 10.731 | 43.595 | 14.961 | 1.00 | 57.92 | A | N | |
| ATOM | 980 | C | LYS | A | 678 | 11.934 | 47.437 | 9.457 | 1.00 | 51.89 | A | C | |
| ATOM | 981 | O | LYS | A | 678 | 13.160 | 47.479 | 9.578 | 1.00 | 51.79 | A | O | |
| ATOM | 982 | N | ARG | A | 679 | 11.283 | 48.253 | 8.630 | 1.00 | 52.05 | A | N | |
| ATOM | 983 | CA | ARG | A | 679 | 12.024 | 49.243 | 7.827 | 1.00 | 52.67 | A | C | |
| ATOM | 984 | CB | ARG | A | 679 | 11.195 | 49.742 | 6.633 | 1.00 | 53.33 | A | C | |
| ATOM | 985 | CG | ARG | A | 679 | 10.676 | 48.672 | 5.682 | 1.00 | 53.84 | A | C | |
| ATOM | 986 | CD | ARG | A | 679 | 10.209 | 49.335 | 4.403 | 1.00 | 53.77 | A | C | |
| ATOM | 987 | NE | ARG | A | 679 | 9.629 | 48.408 | 3.439 | 1.00 | 53.62 | A | N | |
| ATOM | 988 | CZ | ARG | A | 679 | 9.509 | 48.675 | 2.141 | 1.00 | 54.38 | A | C | |
| ATOM | 989 | NH1 | ARG | A | 679 | 9.937 | 49.835 | 1.650 | 1.00 | 53.74 | A | N | |
| ATOM | 990 | NH2 | ARG | A | 679 | 8.945 | 47.789 | 1.330 | 1.00 | 56.06 | A | N | |
| ATOM | 991 | C | ARG | A | 679 | 12.438 | 50.459 | 8.656 | 1.00 | 52.18 | A | C | |
| ATOM | 992 | O | ARG | A | 679 | 11.860 | 51.543 | 8.517 | 1.00 | 51.05 | A | O | |
| ATOM | 993 | N | GLN | A | 696 | 14.557 | 48.720 | 14.957 | 1.00 | 59.40 | A | N | |
| ATOM | 994 | CA | GLN | A | 696 | 15.173 | 49.426 | 13.830 | 1.00 | 60.13 | A | C | |
| ATOM | 995 | CB | GLN | A | 696 | 15.579 | 50.857 | 14.234 | 1.00 | 60.61 | A | C | |
| ATOM | 996 | CG | GLN | A | 696 | 14.413 | 51.836 | 14.405 | 1.00 | 63.46 | A | C | |
| ATOM | 997 | CD | GLN | A | 696 | 13.732 | 52.212 | 13.083 | 1.00 | 65.31 | A | C | |
| ATOM | 998 | OE1 | GLN | A | 696 | 13.650 | 51.400 | 12.150 | 1.00 | 66.02 | A | O | |
| ATOM | 999 | NE2 | GLN | A | 696 | 13.222 | 53.445 | 13.008 | 1.00 | 65.34 | A | N | |
| ATOM | 1000 | C | GLN | A | 696 | 16.405 | 48.681 | 13.326 | 1.00 | 59.31 | A | C | |
| ATOM | 1001 | O | GLN | A | 696 | 17.481 | 48.786 | 13.911 | 1.00 | 59.60 | A | O | |
| ATOM | 1002 | N | LEU | A | 697 | 16.244 | 47.932 | 12.241 | 1.00 | 57.87 | A | N | |
| ATOM | 1003 | CA | LEU | A | 697 | 17.349 | 47.175 | 11.669 | 1.00 | 57.22 | A | C | |
| ATOM | 1004 | CB | LEU | A | 697 | 17.009 | 46.771 | 10.231 | 1.00 | 55.72 | A | C | |
| ATOM | 1005 | CG | LEU | A | 697 | 16.161 | 45.517 | 10.012 | 1.00 | 53.36 | A | C | |
| ATOM | 1006 | CD1 | LEU | A | 697 | 15.385 | 45.627 | 8.723 | 1.00 | 52.05 | A | C | |
| ATOM | 1007 | CD2 | LEU | A | 697 | 17.065 | 44.306 | 9.986 | 1.00 | 53.76 | A | C | |
| ATOM | 1008 | C | LEU | A | 697 | 18.632 | 47.998 | 11.676 | 1.00 | 57.63 | A | C | |
| ATOM | 1009 | O | LEU | A | 697 | 18.575 | 49.228 | 11.652 | 1.00 | 58.68 | A | O | |
| ATOM | 1010 | N | SER | A | 698 | 19.779 | 47.320 | 11.720 | 1.00 | 57.12 | A | N | |
| ATOM | 1011 | CA | SER | A | 698 | 21.080 | 47.993 | 11.699 | 1.00 | 57.41 | A | C | |
| ATOM | 1012 | CB | SER | A | 698 | 21.949 | 47.548 | 12.868 | 1.00 | 57.84 | A | C | |
| ATOM | 1013 | OG | SER | A | 698 | 22.599 | 46.327 | 12.564 | 1.00 | 58.41 | A | O | |
| ATOM | 1014 | C | SER | A | 698 | 21.755 | 47.571 | 10.408 | 1.00 | 57.42 | A | C | |
| ATOM | 1015 | O | SER | A | 698 | 21.358 | 46.578 | 9.811 | 1.00 | 57.31 | A | O | |
| ATOM | 1016 | N | SER | A | 699 | 22.782 | 48.296 | 9.979 | 1.00 | 58.06 | A | N | |
| ATOM | 1017 | CA | SER | A | 699 | 23.477 | 47.924 | 8.735 | 1.00 | 58.95 | A | C | |
| ATOM | 1018 | CB | SER | A | 699 | 24.580 | 48.895 | 8.386 | 1.00 | 60.27 | A | C | |
| ATOM | 1019 | OG | SER | A | 699 | 25.783 | 48.562 | 9.064 | 1.00 | 63.30 | A | O | |
| ATOM | 1020 | C | SER | A | 699 | 24.016 | 46.524 | 8.875 | 1.00 | 58.44 | A | C | |
| ATOM | 1021 | O | SER | A | 699 | 24.173 | 45.812 | 7.883 | 1.00 | 59.32 | A | O | |
| ATOM | 1022 | N | ARG | A | 753 | 24.319 | 46.131 | 10.111 | 1.00 | 57.50 | A | N | |
| ATOM | 1023 | CA | ARG | A | 753 | 24.885 | 44.813 | 10.365 | 1.00 | 56.27 | A | C | |
| ATOM | 1024 | CB | ARG | A | 753 | 25.806 | 44.850 | 11.590 | 1.00 | 57.71 | A | C | |
| ATOM | 1025 | CG | ARG | A | 753 | 27.252 | 44.471 | 11.277 | 1.00 | 60.17 | A | C | |
| ATOM | 1026 | CD | ARG | A | 753 | 27.927 | 43.718 | 12.440 | 1.00 | 62.10 | A | C | |
| ATOM | 1027 | NE | ARG | A | 753 | 27.949 | 44.488 | 13.683 | 1.00 | 62.96 | A | N | |
| ATOM | 1028 | CZ | ARG | A | 753 | 28.454 | 44.047 | 14.832 | 1.00 | 62.84 | A | C | |
| ATOM | 1029 | NH1 | ARG | A | 753 | 28.984 | 42.829 | 14.906 | 1.00 | 61.45 | A | N | |
| ATOM | 1030 | NH2 | ARG | A | 753 | 28.431 | 44.829 | 15.909 | 1.00 | 62.76 | A | N | |
| ATOM | 1031 | C | ARG | A | 753 | 23.835 | 43.713 | 10.537 | 1.00 | 54.44 | A | C | |
| ATOM | 1032 | O | ARG | A | 753 | 24.134 | 42.539 | 10.332 | 1.00 | 54.78 | A | O | |
| ATOM | 1033 | N | ASP | A | 754 | 22.614 | 44.076 | 10.918 | 1.00 | 52.29 | A | N | |
| ATOM | 1034 | CA | ASP | A | 754 | 21.555 | 43.078 | 11.076 | 1.00 | 50.78 | A | C | |
| ATOM | 1035 | CB | ASP | A | 754 | 20.266 | 43.742 | 11.566 | 1.00 | 52.23 | A | C | |
| ATOM | 1036 | CG | ASP | A | 754 | 20.385 | 44.293 | 12.987 | 1.00 | 54.72 | A | C | |
| ATOM | 1037 | OD1 | ASP | A | 754 | 19.756 | 45.344 | 13.272 | 1.00 | 55.78 | A | O | |
| ATOM | 1038 | OD2 | ASP | A | 754 | 21.094 | 43.673 | 13.820 | 1.00 | 54.42 | A | O | |
| ATOM | 1039 | C | ASP | A | 754 | 21.312 | 42.458 | 9.703 | 1.00 | 49.58 | A | C | |
| ATOM | 1040 | O | ASP | A | 754 | 20.951 | 41.284 | 9.575 | 1.00 | 49.18 | A | O | |
| ATOM | 1041 | N | LEU | A | 755 | 21.521 | 43.286 | 8.681 | 1.00 | 48.13 | A | N | |
| ATOM | 1042 | CA | LEU | A | 755 | 21.351 | 42.918 | 7.284 | 1.00 | 45.35 | A | C | |
| ATOM | 1043 | CB | LEU | A | 755 | 21.212 | 44.180 | 6.429 | 1.00 | 44.16 | A | C | |
| ATOM | 1044 | CG | LEU | A | 755 | 20.084 | 45.180 | 6.711 | 1.00 | 42.77 | A | C | |
| ATOM | 1045 | CD1 | LEU | A | 755 | 20.370 | 46.495 | 5.994 | 1.00 | 42.55 | A | C | |
| ATOM | 1046 | CD2 | LEU | A | 755 | 18.756 | 44.607 | 6.254 | 1.00 | 42.98 | A | C | |
| ATOM | 1047 | C | LEU | A | 755 | 22.582 | 42.149 | 6.838 | 1.00 | 45.56 | A | C | |
| ATOM | 1048 | O | LEU | A | 755 | 22.473 | 41.130 | 6.161 | 1.00 | 47.10 | A | O | |
| ATOM | 1049 | N | LEU | A | 756 | 23.756 | 42.660 | 7.204 | 1.00 | 44.57 | A | N | |
| ATOM | 1050 | CA | LEU | A | 756 | 25.028 | 42.027 | 6.860 | 1.00 | 42.03 | A | C | |
| ATOM | 1051 | CB | LEU | A | 756 | 26.174 | 42.747 | 7.578 | 1.00 | 42.56 | A | C | |
| ATOM | 1052 | CG | LEU | A | 756 | 27.247 | 43.411 | 6.698 | 1.00 | 44.02 | A | C | |
| ATOM | 1053 | CD1 | LEU | A | 756 | 26.659 | 43.876 | 5.358 | 1.00 | 44.45 | A | C | |
| ATOM | 1054 | CD2 | LEU | A | 756 | 27.852 | 44.595 | 7.454 | 1.00 | 44.30 | A | C | |
| ATOM | 1055 | C | LEU | A | 756 | 24.955 | 40.563 | 7.273 | 1.00 | 40.49 | A | C | |
| ATOM | 1056 | O | LEU | A | 756 | 25.443 | 39.689 | 6.530 | 1.00 | 39.91 | A | O | |
| ATOM | 1057 | N | HIS | A | 757 | 24.438 | 40.312 | 8.455 | 1.00 | 39.05 | A | N | |
| ATOM | 1058 | CA | HIS | A | 757 | 24.313 | 38.969 | 9.008 | 1.00 | 38.67 | A | C | |
| ATOM | 1059 | CB | HIS | A | 757 | 23.850 | 39.972 | 10.461 | 1.00 | 39.96 | A | C | |
| ATOM | 1060 | CG | HIS | A | 757 | 24.903 | 39.609 | 11.382 | 1.00 | 42.42 | A | C | |
| ATOM | 1061 | CD2 | HIS | A | 757 | 25.132 | 39.395 | 12.701 | 1.00 | 43.15 | A | C | |
| ATOM | 1062 | ND1 | HIS | A | 757 | 25.882 | 40.489 | 10.961 | 1.00 | 44.30 | A | N | |
| ATOM | 1063 | CE1 | HIS | A | 757 | 26.669 | 40.792 | 11.980 | 1.00 | 44.09 | A | C | |
| ATOM | 1064 | NE2 | HIS | A | 757 | 26.236 | 40.141 | 13.048 | 1.00 | 44.91 | A | N | |
| ATOM | 1065 | C | HIS | A | 757 | 23.368 | 38.083 | 8.186 | 1.00 | 37.46 | A | C | |
| ATOM | 1066 | O | HIS | A | 757 | 23.668 | 36.913 | 7.914 | 1.00 | 38.54 | A | O | |
| ATOM | 1067 | N | PHE | A | 758 | 22.228 | 38.631 | 7.788 | 1.00 | 35.07 | A | N | |
| ATOM | 1068 | CA | PHE | A | 758 | 21.298 | 37.876 | 6.693 | 1.00 | 33.92 | A | C | |
| ATOM | 1069 | CB | PHE | A | 758 | 20.111 | 38.760 | 6.553 | 1.00 | 34.57 | A | C | |
| ATOM | 1070 | CG | PHE | A | 758 | 19.288 | 39.292 | 7.710 | 1.00 | 34.94 | A | C | |
| ATOM | 1071 | CD1 | PHE | A | 758 | 18.420 | 40.364 | 7.513 | 1.00 | 34.35 | A | C | |
| ATOM | 1072 | CD2 | PHE | A | 758 | 19.356 | 38.713 | 8.975 | 1.00 | 35.86 | A | C | |
| ATOM | 1073 | CE1 | PHE | A | 758 | 17.634 | 40.849 | 8.551 | 1.00 | 35.83 | A | C | |
| ATOM | 1074 | CE2 | PHE | A | 758 | 18.570 | 39.191 | 10.027 | 1.00 | 36.98 | A | C | |
| ATOM | 1075 | CZ | PHE | A | 758 | 17.707 | 40.261 | 9.815 | 1.00 | 37.14 | A | C | |
| ATOM | 1076 | C | PHE | A | 758 | 22.071 | 37.450 | 5.698 | 1.00 | 33.32 | A | C | |
| ATOM | 1077 | O | PHE | A | 758 | 22.102 | 36.260 | 5.324 | 1.00 | 30.94 | A | O | |
| ATOM | 1078 | N | SER | A | 759 | 22.700 | 38.440 | 5.058 | 1.00 | 33.23 | A | N | |
| ATOM | 1079 | CA | SER | A | 759 | 23.479 | 38.233 | 3.835 | 1.00 | 33.64 | A | C | |
| ATOM | 1080 | CB | SER | A | 759 | 24.210 | 39.513 | 3.438 | 1.00 | 32.58 | A | C | |
| ATOM | 1081 | OG | SER | A | 759 | 23.307 | 40.418 | 2.839 | 1.00 | 33.68 | A | O | |
| ATOM | 1082 | C | SER | A | 759 | 24.491 | 37.098 | 3.896 | 1.00 | 34.39 | A | C | |
| ATOM | 1083 | O | SER | A | 759 | 24.571 | 36.294 | 2.965 | 1.00 | 34.14 | A | O | |
| ATOM | 1084 | N | SER | A | 760 | 25.274 | 37.021 | 4.965 | 1.00 | 34.05 | A | N | |
| ATOM | 1085 | CA | SER | A | 760 | 26.244 | 35.950 | 5.028 | 1.00 | 34.77 | A | C | |
| ATOM | 1086 | CB | SER | A | 760 | 27.384 | 36.324 | 5.954 | 1.00 | 35.70 | A | C | |
| ATOM | 1087 | OG | SER | A | 760 | 28.156 | 37.322 | 5.312 | 1.00 | 37.17 | A | O | |
| ATOM | 1088 | C | SER | A | 760 | 25.632 | 34.621 | 5.426 | 1.00 | 35.09 | A | C | |
| ATOM | 1089 | O | SER | A | 760 | 25.948 | 33.580 | 4.826 | 1.00 | 34.56 | A | O | |
| ATOM | 1090 | N | GLN | A | 761 | 24.757 | 34.639 | 6.423 | 1.00 | 35.27 | A | N | |
| ATOM | 1091 | CA | GLN | A | 761 | 24.114 | 33.398 | 6.827 | 1.00 | 36.23 | A | C | |
| ATOM | 1092 | CB | GLN | A | 761 | 22.999 | 33.689 | 7.801 | 1.00 | 36.55 | A | C | |
| ATOM | 1093 | CG | GLN | A | 761 | 23.495 | 34.221 | 9.101 | 1.00 | 38.95 | A | C | |
| ATOM | 1094 | CD | GLN | A | 761 | 22.377 | 34.847 | 9.865 | 1.00 | 42.10 | A | C | |
| ATOM | 1095 | OE1 | GLN | A | 761 | 21.910 | 35.935 | 9.517 | 1.00 | 44.25 | A | O | |
| ATOM | 1096 | NE2 | GLN | A | 761 | 21.903 | 34.156 | 10.896 | 1.00 | 43.26 | A | N | |
| ATOM | 1097 | C | GLN | A | 761 | 23.552 | 32.689 | 5.589 | 1.00 | 35.95 | A | C | |
| ATOM | 1098 | O | GLN | A | 761 | 23.942 | 31.556 | 5.283 | 1.00 | 37.99 | A | O | |
| ATOM | 1099 | N | VAL | A | 762 | 22.653 | 33.351 | 4.867 | 1.00 | 33.11 | A | N | |
| ATOM | 1100 | CA | VAL | A | 762 | 22.094 | 32.740 | 3.669 | 1.00 | 31.49 | A | C | |
| ATOM | 1101 | CB | VAL | A | 762 | 21.143 | 33.722 | 2.934 | 1.00 | 30.83 | A | C | |
| ATOM | 1102 | CG1 | VAL | A | 762 | 20.662 | 33.117 | 1.657 | 1.00 | 30.39 | A | C | |
| ATOM | 1103 | CG2 | VAL | A | 762 | 19.933 | 34.048 | 3.808 | 1.00 | 31.70 | A | C | |
| ATOM | 1104 | C | VAL | A | 762 | 23.219 | 32.308 | 2.706 | 1.00 | 30.40 | A | C | |
| ATOM | 1105 | O | VAL | A | 762 | 23.146 | 31.255 | 2.056 | 1.00 | 29.86 | A | O | |
| ATOM | 1106 | N | ALA | A | 763 | 24.270 | 33.117 | 2.614 | 1.00 | 30.05 | A | N | |
| ATOM | 1107 | CA | ALA | A | 763 | 25.363 | 32.801 | 1.704 | 1.00 | 28.49 | A | C | |
| ATOM | 1108 | CB | ALA | A | 763 | 26.334 | 33.958 | 1.636 | 1.00 | 24.87 | A | C | |
| ATOM | 1109 | C | ALA | A | 763 | 26.041 | 31.548 | 2.232 | 1.00 | 29.71 | A | C | |
| ATOM | 1110 | O | ALA | A | 763 | 26.613 | 30.754 | 1.471 | 1.00 | 30.02 | A | O | |
| ATOM | 1111 | N | GLN | A | 764 | 25.972 | 31.366 | 3.546 | 1.00 | 29.59 | A | N | |
| ATOM | 1112 | CA | GLN | A | 764 | 26.564 | 30.189 | 4.151 | 1.00 | 29.26 | A | C | |
| ATOM | 1113 | CB | GLN | A | 764 | 26.532 | 30.289 | 5.668 | 1.00 | 29.11 | A | C | |
| ATOM | 1114 | CG | GLN | A | 764 | 27.689 | 31.079 | 6.195 | 1.00 | 33.57 | A | C | |
| ATOM | 1115 | CD | GLN | A | 764 | 27.646 | 31.200 | 7.685 | 1.00 | 36.30 | A | C | |
| ATOM | 1116 | OE1 | GLN | A | 764 | 27.433 | 30.206 | 8.389 | 1.00 | 38.99 | A | O | |
| ATOM | 1117 | NE2 | GLN | A | 764 | 27.851 | 32.421 | 8.192 | 1.00 | 38.37 | A | N | |
| ATOM | 1118 | C | GLN | A | 764 | 25.793 | 28.970 | 3.688 | 1.00 | 28.58 | A | C | |
| ATOM | 1119 | O | GLN | A | 764 | 26.368 | 38.080 | 3.043 | 1.00 | 30.46 | A | O | |
| ATOM | 1120 | N | GLY | A | 765 | 24.498 | 28.928 | 4.014 | 1.00 | 25.79 | A | N | |
| ATOM | 1121 | CA | GLY | A | 765 | 23.666 | 27.812 | 3.593 | 1.00 | 22.81 | A | C | |
| ATOM | 1122 | C | GLY | A | 765 | 23.985 | 27.465 | 2.152 | 1.00 | 20.77 | A | C | |
| ATOM | 1123 | O | GLY | A | 765 | 24.415 | 26.355 | 1.861 | 1.00 | 22.11 | A | O | |
| ATOM | 1124 | N | MET | A | 766 | 23.804 | 28.424 | 1.249 | 1.00 | 20.46 | A | N | |
| ATOM | 1125 | CA | MET | A | 766 | 24.100 | 28.214 | −0.166 | 1.00 | 20.55 | A | C | |
| ATOM | 1126 | CB | MET | A | 766 | 24.013 | 29.544 | −0.906 | 1.00 | 19.86 | A | C | |
| ATOM | 1127 | CG | MET | A | 766 | 22.597 | 30.100 | −0.927 | 1.00 | 19.64 | A | C | |
| ATOM | 1128 | SD | MET | A | 766 | 21.455 | 28.787 | −1.464 | 1.00 | 16.58 | A | S | |
| ATOM | 1129 | CE | MET | A | 766 | 21.868 | 28.634 | −3.237 | 1.00 | 17.84 | A | C | |
| ATOM | 1130 | C | MET | A | 766 | 25.476 | 27.567 | −0.365 | 1.00 | 21.32 | A | C | |
| ATOM | 1131 | O | MET | A | 766 | 25.616 | 26.623 | −1.147 | 1.00 | 22.29 | A | O | |
| ATOM | 1132 | N | ALA | A | 767 | 26.487 | 28.062 | 0.346 | 1.00 | 22.01 | A | N | |
| ATOM | 1133 | CA | ALA | A | 767 | 27.826 | 27.480 | 0.268 | 1.00 | 21.37 | A | C | |
| ATOM | 1134 | CB | ALA | A | 767 | 28.737 | 28.115 | 1.313 | 1.00 | 21.12 | A | C | |
| ATOM | 1135 | C | ALA | A | 767 | 27.686 | 25.982 | 0.544 | 1.00 | 22.44 | A | C | |
| ATOM | 1136 | O | ALA | A | 767 | 28.309 | 25.150 | −0.136 | 1.00 | 23.19 | A | O | |
| ATOM | 1137 | N | PHE | A | 768 | 26.850 | 26.561 | 1.533 | 1.00 | 22.41 | A | N | |
| ATOM | 1138 | CA | PHE | A | 768 | 26.583 | 24.464 | 1.934 | 1.00 | 22.61 | A | C | |
| ATOM | 1139 | CB | PHE | A | 768 | 25.682 | 24.243 | 3.154 | 1.00 | 20.10 | A | C | |
| ATOM | 1140 | CG | PHE | A | 768 | 25.559 | 22.906 | 3.783 | 1.00 | 20.92 | A | C | |
| ATOM | 1141 | CD1 | PHE | A | 768 | 26.654 | 22.320 | 4.419 | 1.00 | 19.31 | A | C | |
| ATOM | 1142 | CD2 | PHE | A | 768 | 24.336 | 22.232 | 3.781 | 1.00 | 21.35 | A | C | |
| ATOM | 1143 | CE1 | PHE | A | 768 | 26.537 | 21.088 | 5.048 | 1.00 | 15.93 | A | C | |
| ATOM | 1144 | CE2 | PHE | A | 768 | 24.204 | 20.989 | 4.411 | 1.00 | 17.40 | A | C | |
| ATOM | 1145 | CZ | PHE | A | 768 | 25.314 | 20.426 | 5.043 | 1.00 | 17.68 | A | C | |
| ATOM | 1146 | C | PHE | A | 768 | 25.909 | 23.454 | 0.828 | 1.00 | 24.17 | A | C | |
| ATOM | 1147 | O | PHE | A | 768 | 26.451 | 22.439 | 0.361 | 1.00 | 26.26 | A | O | |
| ATOM | 1148 | N | LEU | A | 769 | 24.724 | 23.895 | 0.406 | 1.00 | 24.65 | A | N | |
| ATOM | 1149 | CA | LEU | A | 769 | 24.011 | 23.182 | −0.652 | 1.00 | 25.15 | A | C | |
| ATOM | 1150 | CB | LEU | A | 769 | 22.769 | 23.964 | −1.108 | 1.00 | 24.29 | A | C | |
| ATOM | 1151 | CG | LEU | A | 769 | 21.635 | 24.103 | −0.071 | 1.00 | 23.39 | A | C | |
| ATOM | 1152 | CD1 | LEU | A | 769 | 20.390 | 24.620 | −0.780 | 1.00 | 22.16 | A | C | |
| ATOM | 1153 | CD2 | LEU | A | 769 | 21.319 | 22.749 | 0.596 | 1.00 | 21.69 | A | C | |
| ATOM | 1154 | C | LEU | A | 769 | 24.992 | 23.012 | −1.797 | 1.00 | 25.54 | A | C | |
| ATOM | 1155 | O | LEU | A | 769 | 25.057 | 21.954 | −2.423 | 1.00 | 27.42 | A | O | |
| ATOM | 1156 | N | ALA | A | 770 | 25.782 | 24.051 | −2.050 | 1.00 | 24.75 | A | N | |
| ATOM | 1157 | CA | ALA | A | 770 | 26.785 | 23.987 | −3.098 | 1.00 | 22.83 | A | C | |
| ATOM | 1158 | CB | ALA | A | 770 | 27.625 | 25.230 | −3.075 | 1.00 | 22.71 | A | C | |
| ATOM | 1159 | C | ALA | A | 770 | 27.680 | 22.761 | −2.907 | 1.00 | 23.22 | A | C | |
| ATOM | 1160 | O | ALA | A | 770 | 27.842 | 21.969 | −3.836 | 1.00 | 21.83 | A | O | |
| ATOM | 1161 | N | SER | A | 771 | 28.247 | 22.597 | −1.701 | 1.00 | 23.57 | A | N | |
| ATOM | 1162 | CA | SER | A | 771 | 29.154 | 21.469 | −1.412 | 1.00 | 23.96 | A | C | |
| ATOM | 1163 | CB | SER | A | 771 | 30.031 | 21.773 | −0.186 | 1.00 | 21.46 | A | C | |
| ATOM | 1164 | OG | SER | A | 771 | 29.280 | 21.787 | 1.011 | 1.00 | 16.59 | A | O | |
| ATOM | 1165 | C | SER | A | 771 | 28.433 | 20.144 | −1.190 | 1.00 | 25.73 | A | C | |
| ATOM | 1166 | O | SER | A | 771 | 28.661 | 19.455 | −0.186 | 1.00 | 27.35 | A | O | |
| ATOM | 1167 | N | LYS | A | 772 | 27.558 | 19.808 | −2.132 | 1.00 | 26.55 | A | N | |
| ATOM | 1168 | CA | LYS | A | 772 | 26.759 | 18.583 | −2.114 | 1.00 | 26.30 | A | C | |
| ATOM | 1169 | CB | LYS | A | 772 | 25.597 | 18.654 | −1.108 | 1.00 | 24.46 | A | C | |
| ATOM | 1170 | CG | LYS | A | 772 | 25.960 | 18.945 | 0.327 | 1.00 | 23.55 | A | C | |
| ATOM | 1171 | CD | LYS | A | 772 | 26.709 | 17.801 | 0.979 | 1.00 | 25.55 | A | C | |
| ATOM | 1172 | CE | LYS | A | 772 | 26.882 | 18.047 | 2.470 | 1.00 | 27.02 | A | C | |
| ATOM | 1173 | NZ | LYS | A | 772 | 27.695 | 16.990 | 3.143 | 1.00 | 26.21 | A | N | |
| ATOM | 1174 | C | LYS | A | 772 | 26.154 | 18.517 | −3.501 | 1.00 | 27.13 | A | C | |
| ATOM | 1175 | O | LYS | A | 772 | 25.215 | 17.765 | −3.725 | 1.00 | 28.56 | A | O | |
| ATOM | 1176 | N | ASN | A | 773 | 26.685 | 19.322 | −4.421 | 1.00 | 28.70 | A | N | |
| ATOM | 1177 | CA | ASN | A | 773 | 26.176 | 19.367 | −5.797 | 1.00 | 30.22 | A | C | |
| ATOM | 1178 | CB | ASN | A | 773 | 26.571 | 18.105 | −6.569 | 1.00 | 29.39 | A | C | |
| ATOM | 1179 | CG | ASN | A | 773 | 28.067 | 17.945 | −6.701 | 1.00 | 30.63 | A | C | |
| ATOM | 1180 | OD1 | ASN | A | 773 | 28.839 | 18.770 | −6.195 | 1.00 | 30.99 | A | O | |
| ATOM | 1181 | ND2 | ASN | A | 773 | 28.495 | 16.875 | −7.386 | 1.00 | 31.19 | A | N | |
| ATOM | 1182 | C | ASN | A | 773 | 24.661 | 19.439 | −5.713 | 1.00 | 30.44 | A | C | |
| ATOM | 1183 | O | ASN | A | 773 | 23.957 | 18.589 | −6.267 | 1.00 | 33.19 | A | O | |
| ATOM | 1184 | N | CYS | A | 774 | 24.155 | 20.426 | −4.989 | 1.00 | 29.26 | A | N | |
| ATOM | 1185 | CA | CYS | A | 774 | 22.712 | 20.568 | −4.838 | 1.00 | 29.03 | A | C | |
| ATOM | 1186 | CB | CYS | A | 774 | 22.296 | 20.297 | −3.390 | 1.00 | 28.88 | A | C | |
| ATOM | 1187 | SG | CYS | A | 774 | 20.560 | 20.659 | −3.012 | 1.00 | 22.93 | A | S | |
| ATOM | 1188 | C | CYS | A | 774 | 22.264 | 21.951 | −5.218 | 1.00 | 29.34 | A | C | |
| ATOM | 1189 | O | CYS | A | 774 | 22.557 | 22.907 | −4.509 | 1.00 | 29.01 | A | O | |
| ATOM | 1190 | N | ILE | A | 775 | 21.558 | 22.058 | −6.338 | 1.00 | 30.96 | A | N | |
| ATOM | 1191 | CA | ILE | A | 775 | 21.054 | 23.355 | −6.798 | 1.00 | 32.07 | A | C | |
| ATOM | 1192 | CB | ILE | A | 775 | 20.987 | 23.417 | −8.344 | 1.00 | 33.69 | A | C | |
| ATOM | 1193 | CG2 | ILE | A | 775 | 22.339 | 22.990 | −8.945 | 1.00 | 32.43 | A | C | |
| ATOM | 1194 | CG1 | ILE | A | 775 | 19.867 | 22.508 | −8.859 | 1.00 | 33.62 | A | C | |
| ATOM | 1195 | CD1 | ILE | A | 775 | 19.718 | 22.565 | −10.369 | 1.00 | 34.80 | A | C | |
| ATOM | 1196 | C | ILE | A | 775 | 19.651 | 23.633 | −6.218 | 1.00 | 31.07 | A | C | |
| ATOM | 1197 | O | ILE | A | 775 | 18.818 | 22.723 | −6.113 | 1.00 | 31.57 | A | O | |
| ATOM | 1198 | N | HIS | A | 776 | 19.421 | 24.893 | −5.851 | 1.00 | 28.28 | A | N | |
| ATOM | 1199 | CA | HIS | A | 776 | 18.175 | 25.372 | −5.251 | 1.00 | 26.67 | A | C | |
| ATOM | 1200 | CB | HIS | A | 776 | 18.509 | 26.621 | −4.411 | 1.00 | 26.79 | A | C | |
| ATOM | 1201 | CG | HIS | A | 776 | 17.495 | 26.968 | −3.361 | 1.00 | 25.14 | A | C | |
| ATOM | 1202 | CD2 | HIS | A | 776 | 17.451 | 26.679 | −2.035 | 1.00 | 23.62 | A | C | |
| ATOM | 1203 | ND1 | HIS | A | 776 | 16.392 | 27.758 | −3.615 | 1.00 | 24.32 | A | N | |
| ATOM | 1204 | CE1 | HIS | A | 776 | 15.714 | 27.938 | −2.492 | 1.00 | 22.67 | A | C | |
| ATOM | 1205 | NE2 | HIS | A | 776 | 16.334 | 27.292 | −1.519 | 1.00 | 20.45 | A | N | |
| ATOM | 1206 | C | HIS | A | 776 | 17.161 | 25.709 | −6.355 | 1.00 | 26.75 | A | C | |
| ATOM | 1207 | O | HIS | A | 776 | 16.036 | 25.204 | −6.368 | 1.00 | 28.37 | A | O | |
| ATOM | 1208 | N | ARG | A | 777 | 16.702 | 26.566 | −7.284 | 1.00 | 25.81 | A | N | |
| ATOM | 1209 | CA | ARG | A | 777 | 16.702 | 26.970 | −8.387 | 1.00 | 25.45 | A | C | |
| ATOM | 1210 | CB | ARG | A | 777 | 16.063 | 25.743 | −9.068 | 1.00 | 25.56 | A | C | |
| ATOM | 1211 | CG | ARG | A | 777 | 17.055 | 24.642 | −9.438 | 1.00 | 25.44 | A | C | |
| ATOM | 1212 | CD | ARG | A | 777 | 16.587 | 23.737 | −10.608 | 1.00 | 26.98 | A | C | |
| ATOM | 1213 | NE | ARG | A | 777 | 15.403 | 22.899 | −10.367 | 1.00 | 27.69 | A | N | |
| ATOM | 1214 | CZ | ARG | A | 777 | 15.009 | 21.911 | −11.182 | 1.00 | 28.74 | A | C | |
| ATOM | 1215 | NH1 | ARG | A | 777 | 15.703 | 21.623 | −12.283 | 1.00 | 28.01 | A | N | |
| ATOM | 1216 | NH2 | ARG | A | 777 | 13.904 | 21.218 | −10.920 | 1.00 | 28.98 | A | N | |
| ATOM | 1217 | C | ARG | A | 777 | 15.607 | 27.964 | −7.959 | 1.00 | 26.63 | A | C | |
| ATOM | 1218 | O | ARG | A | 777 | 14.820 | 28.441 | −8.809 | 1.00 | 27.57 | A | O | |
| ATOM | 1219 | N | ASP | A | 778 | 15.532 | 28.290 | −6.667 | 1.00 | 25.07 | A | N | |
| ATOM | 1220 | CA | ASP | A | 778 | 14.517 | 29.262 | −6.252 | 1.00 | 25.74 | A | C | |
| ATOM | 1221 | CB | ASP | A | 778 | 13.219 | 28.591 | −5.814 | 1.00 | 25.07 | A | C | |
| ATOM | 1222 | CG | ASP | A | 778 | 12.067 | 29.578 | −5.744 | 1.00 | 24.70 | A | C | |
| ATOM | 1223 | OD1 | ASP | A | 778 | 10.948 | 29.167 | −5.382 | 1.00 | 26.02 | A | O | |
| ATOM | 1224 | OD2 | ASP | A | 778 | 12.284 | 30.774 | −6.063 | 1.00 | 24.59 | A | O | |
| ATOM | 1225 | C | ASP | A | 778 | 14.944 | 30.189 | −5.142 | 1.00 | 26.73 | A | C | |
| ATOM | 1226 | O | ASP | A | 778 | 14.191 | 30.390 | −4.182 | 1.00 | 26.97 | A | O | |
| ATOM | 1227 | N | VAL | A | 779 | 16.141 | 30.756 | −5.259 | 1.00 | 27.60 | A | N | |
| ATOM | 1228 | CA | VAL | A | 779 | 16.599 | 31.666 | −4.226 | 1.00 | 29.25 | A | C | |
| ATOM | 1229 | CB | VAL | A | 779 | 18.104 | 31.908 | −4.312 | 1.00 | 29.28 | A | C | |
| ATOM | 1230 | CG1 | VAL | A | 779 | 18.478 | 33.082 | −3.429 | 1.00 | 29.41 | A | C | |
| ATOM | 1231 | CG2 | VAL | A | 779 | 18.850 | 30.644 | −3.857 | 1.00 | 26.69 | A | C | |
| ATOM | 1232 | C | VAL | A | 779 | 15.850 | 32.983 | −4.356 | 1.00 | 30.11 | A | C | |
| ATOM | 1233 | O | VAL | A | 779 | 15.727 | 33.530 | −5.457 | 1.00 | 31.42 | A | O | |
| ATOM | 1234 | N | ALA | A | 780 | 15.322 | 33.464 | −3.224 | 1.00 | 30.18 | A | N | |
| ATOM | 1235 | CA | ALA | A | 780 | 14.556 | 34.705 | −3.149 | 1.00 | 30.18 | A | C | |
| ATOM | 1236 | CB | ALA | A | 780 | 13.268 | 34.574 | −3.962 | 1.00 | 28.98 | A | C | |
| ATOM | 1237 | C | ALA | A | 780 | 14.236 | 34.958 | −1.667 | 1.00 | 32.26 | A | C | |
| ATOM | 1238 | O | ALA | A | 780 | 14.119 | 34.014 | −0.861 | 1.00 | 33.04 | A | O | |
| ATOM | 1239 | N | ALA | A | 781 | 14.082 | 36.225 | −1.295 | 1.00 | 32.78 | A | N | |
| ATOM | 1240 | CA | ALA | A | 781 | 13.848 | 36.524 | 0.111 | 1.00 | 31.09 | A | C | |
| ATOM | 1241 | CB | ALA | A | 781 | 13.607 | 38.046 | 0.275 | 1.00 | 31.62 | A | C | |
| ATOM | 1242 | C | ALA | A | 781 | 12.706 | 35.732 | 0.728 | 1.00 | 29.29 | A | C | |
| ATOM | 1243 | O | ALA | A | 781 | 12.749 | 35.375 | 1.905 | 1.00 | 27.82 | A | O | |
| ATOM | 1244 | N | ARG | A | 782 | 11.964 | 35.421 | −0.072 | 1.00 | 28.36 | A | N | |
| ATOM | 1245 | CA | ARG | A | 782 | 10.560 | 34.667 | 0.439 | 1.00 | 26.36 | A | C | |
| ATOM | 1246 | CB | ARG | A | 782 | 9.450 | 34.609 | −0.620 | 1.00 | 23.06 | A | C | |
| ATOM | 1247 | CG | ARG | A | 782 | 9.914 | 34.118 | −1.987 | 1.00 | 21.59 | A | C | |
| ATOM | 1248 | CD | ARG | A | 782 | 8.752 | 33.891 | −2.925 | 1.00 | 19.55 | A | C | |
| ATOM | 1249 | NE | ARG | A | 782 | 9.169 | 33.292 | −4.186 | 1.00 | 19.28 | A | N | |
| ATOM | 1250 | CZ | ARG | A | 782 | 9.942 | 33.893 | −5.087 | 1.00 | 22.50 | A | C | |
| ATOM | 1251 | NH1 | ARG | A | 782 | 10.394 | 35.125 | −4.875 | 1.00 | 23.14 | A | N | |
| ATOM | 1252 | NH2 | ARG | A | 782 | 10.267 | 33.261 | −6.213 | 1.00 | 24.84 | A | N | |
| ATOM | 1253 | C | ARG | A | 782 | 10.927 | 33.253 | 0.921 | 1.00 | 26.61 | A | C | |
| ATOM | 1254 | O | ARG | A | 782 | 10.224 | 32.686 | 1.760 | 1.00 | 28.52 | A | O | |
| ATOM | 1255 | N | ASN | A | 783 | 12.017 | 32.684 | 0.413 | 1.00 | 25.41 | A | N | |
| ATOM | 1256 | CA | ASN | A | 783 | 12.422 | 31.337 | 0.819 | 1.00 | 24.69 | A | C | |
| ATOM | 1257 | CB | ASN | A | 783 | 12.974 | 30.542 | −0.366 | 1.00 | 26.52 | A | C | |
| ATOM | 1258 | CG | ASN | A | 783 | 11.889 | 30.043 | −1.286 | 1.00 | 28.60 | A | C | |
| ATOM | 1259 | OD1 | ASN | A | 783 | 10.828 | 29.584 | −0.831 | 1.00 | 29.47 | A | O | |
| ATOM | 1260 | ND2 | ASN | A | 783 | 12.147 | 30.109 | −2.594 | 1.00 | 29.33 | A | N | |
| ATOM | 1261 | C | ASN | A | 783 | 13.464 | 31.316 | 1.920 | 1.00 | 24.79 | A | C | |
| ATOM | 1262 | O | ASN | A | 783 | 14.231 | 30.343 | 2.047 | 1.00 | 26.13 | A | O | |
| ATOM | 1263 | N | VAL | A | 784 | 13.530 | 32.401 | 2.683 | 1.00 | 24.11 | A | N | |
| ATOM | 1264 | CA | VAL | A | 784 | 14.454 | 32.492 | 3.801 | 1.00 | 21.92 | A | C | |
| ATOM | 1265 | CB | VAL | A | 784 | 15.379 | 33.683 | 3.727 | 1.00 | 18.04 | A | C | |
| ATOM | 1266 | CG1 | VAL | A | 784 | 16.267 | 33.698 | 4.958 | 1.00 | 17.80 | A | C | |
| ATOM | 1267 | CG2 | VAL | A | 784 | 16.214 | 33.617 | 2.488 | 1.00 | 17.69 | A | C | |
| ATOM | 1268 | C | VAL | A | 784 | 13.599 | 32.686 | 5.035 | 1.00 | 24.87 | A | C | |
| ATOM | 1269 | O | VAL | A | 784 | 12.660 | 33.503 | 5.050 | 1.00 | 26.53 | A | O | |
| ATOM | 1270 | N | LEU | A | 785 | 13.917 | 31.921 | 6.068 | 1.00 | 25.17 | A | N | |
| ATOM | 1271 | CA | LEU | A | 785 | 13.194 | 32.000 | 7.309 | 1.00 | 23.73 | A | C | |
| ATOM | 1272 | CB | LEU | A | 785 | 12.729 | 30.612 | 7.722 | 1.00 | 24.02 | A | C | |
| ATOM | 1273 | CG | LEU | A | 785 | 11.217 | 30.440 | 7.798 | 1.00 | 24.49 | A | C | |
| ATOM | 1274 | CD1 | LEU | A | 785 | 10.551 | 31.107 | 6.596 | 1.00 | 24.92 | A | C | |
| ATOM | 1275 | CD2 | LEU | A | 785 | 10.902 | 28.959 | 7.839 | 1.00 | 25.29 | A | C | |
| ATOM | 1276 | C | LEU | A | 785 | 14.167 | 32.514 | 8.328 | 1.00 | 24.62 | A | C | |
| ATOM | 1277 | O | LEU | A | 785 | 15.395 | 32.380 | 8.152 | 1.00 | 23.48 | A | O | |
| ATOM | 1278 | N | LEU | A | 786 | 13.622 | 33.113 | 9.383 | 1.00 | 25.43 | A | N | |
| ATOM | 1279 | CA | LEU | A | 786 | 14.432 | 33.601 | 10.489 | 1.00 | 26.09 | A | C | |
| ATOM | 1280 | CB | LEU | A | 786 | 14.160 | 35.070 | 10.758 | 1.00 | 25.39 | A | C | |
| ATOM | 1281 | CG | LEU | A | 786 | 14.380 | 36.036 | 9.613 | 1.00 | 25.91 | A | C | |
| ATOM | 1282 | CD1 | LEU | A | 786 | 14.584 | 37.440 | 10.203 | 1.00 | 27.81 | A | C | |
| ATOM | 1283 | CD2 | LEU | A | 786 | 15.600 | 35.632 | 8.831 | 1.00 | 25.86 | A | C | |
| ATOM | 1284 | C | LEU | A | 786 | 14.066 | 32.775 | 11.738 | 1.00 | 27.31 | A | C | |
| ATOM | 1285 | O | LEU | A | 786 | 12.882 | 32.626 | 12.092 | 1.00 | 26.32 | A | O | |
| ATOM | 1286 | N | THR | A | 787 | 15.081 | 32.223 | 12.392 | 1.00 | 28.25 | A | N | |
| ATOM | 1287 | CA | THR | A | 787 | 14.843 | 31.438 | 13.584 | 1.00 | 30.47 | A | C | |
| ATOM | 1288 | CB | THR | A | 787 | 15.540 | 30.112 | 13.485 | 1.00 | 29.89 | A | C | |
| ATOM | 1289 | CG1 | THR | A | 787 | 15.312 | 29.375 | 14.693 | 1.00 | 30.51 | A | O | |
| ATOM | 1290 | CG2 | THR | A | 787 | 17.032 | 30.332 | 13.276 | 1.00 | 29.64 | A | C | |
| ATOM | 1291 | C | THR | A | 787 | 15.354 | 32.183 | 14.825 | 1.00 | 32.31 | A | C | |
| ATOM | 1292 | O | THR | A | 787 | 15.623 | 33.387 | 14.771 | 1.00 | 33.50 | A | O | |
| ATOM | 1293 | N | ASN | A | 788 | 15.497 | 31.479 | 15.942 | 1.00 | 33.65 | A | N | |
| ATOM | 1294 | CA | ASN | A | 788 | 15.969 | 32.122 | 17.164 | 1.00 | 34.49 | A | C | |
| ATOM | 1295 | CB | ASN | A | 788 | 16.153 | 31.087 | 18.270 | 1.00 | 34.64 | A | C | |
| ATOM | 1296 | CG | ASN | A | 788 | 15.025 | 30.088 | 18.313 | 1.00 | 35.28 | A | C | |
| ATOM | 1297 | OD1 | ASN | A | 788 | 13.860 | 30.443 | 18.125 | 1.00 | 36.48 | A | O | |
| ATOM | 1298 | ND2 | ASN | A | 788 | 15.360 | 28.826 | 18.564 | 1.00 | 37.16 | A | N | |
| ATOM | 1299 | C | ASN | A | 788 | 17.270 | 32.900 | 16.957 | 1.00 | 35.10 | A | C | |
| ATOM | 1300 | O | ASN | A | 788 | 18.237 | 32.394 | 16.368 | 1.00 | 35.22 | A | O | |
| ATOM | 1301 | N | GLY | A | 789 | 17.280 | 34.135 | 17.459 | 1.00 | 36.15 | A | N | |
| ATOM | 1302 | CA | GLY | A | 789 | 18.443 | 34.993 | 17.333 | 1.00 | 35.99 | A | C | |
| ATOM | 1303 | C | GLY | A | 789 | 18.422 | 35.595 | 15.948 | 1.00 | 37.63 | A | C | |
| ATOM | 1304 | O | GLY | A | 789 | 19.415 | 36.182 | 15.501 | 1.00 | 38.64 | A | O | |
| ATOM | 1305 | N | HIS | A | 790 | 17.280 | 35.449 | 15.266 | 1.00 | 37.25 | A | N | |
| ATOM | 1306 | CA | HIS | A | 790 | 17.120 | 35.966 | 13.909 | 1.00 | 36.21 | A | C | |
| ATOM | 1307 | CB | HIS | A | 790 | 17.227 | 37.488 | 13.931 | 1.00 | 37.45 | A | C | |
| ATOM | 1308 | CG | HIS | A | 790 | 16.076 | 38.161 | 14.605 | 1.00 | 39.57 | A | C | |
| ATOM | 1309 | CD2 | HIS | A | 790 | 15.984 | 38.781 | 15.804 | 1.00 | 40.04 | A | C | |
| ATOM | 1310 | ND1 | HIS | A | 790 | 14.820 | 38.240 | 14.033 | 1.00 | 41.90 | A | N | |
| ATOM | 1311 | CE1 | HIS | A | 790 | 14.005 | 38.882 | 14.851 | 1.00 | 40.87 | A | C | |
| ATOM | 1312 | NE2 | HIS | A | 790 | 14.687 | 39.222 | 14.932 | 1.00 | 41.48 | A | N | |
| ATOM | 1313 | C | HIS | A | 790 | 18.203 | 35.366 | 12.990 | 1.00 | 35.04 | A | C | |
| ATOM | 1314 | O | HIS | A | 790 | 18.915 | 36.089 | 12.277 | 1.00 | 34.98 | A | O | |
| ATOM | 1315 | N | VAL | A | 791 | 18.337 | 34.045 | 13.021 | 1.00 | 32.48 | A | N | |
| ATOM | 1316 | CA | VAL | A | 791 | 19.336 | 33.384 | 12.198 | 1.00 | 31.11 | A | C | |
| ATOM | 1317 | CB | VAL | A | 791 | 20.033 | 32.229 | 12.970 | 1.00 | 28.98 | A | C | |
| ATOM | 1318 | CG1 | VAL | A | 791 | 21.023 | 31.526 | 12.062 | 1.00 | 26.43 | A | C | |
| ATOM | 1319 | CG2 | VAL | A | 791 | 20.747 | 32.783 | 14.198 | 1.00 | 26.95 | A | C | |
| ATOM | 1320 | C | VAL | A | 791 | 18.669 | 32.848 | 10.938 | 1.00 | 31.18 | A | C | |
| ATOM | 1321 | O | VAL | A | 791 | 17.774 | 31.996 | 11.005 | 1.00 | 32.21 | A | O | |
| ATOM | 1322 | N | ALA | A | 792 | 19.096 | 33.345 | 9.784 | 1.00 | 29.28 | A | N | |
| ATOM | 1323 | CA | ALA | A | 792 | 18.483 | 32.887 | 8.550 | 1.00 | 29.24 | A | C | |
| ATOM | 1324 | CB | ALA | A | 792 | 18.995 | 33.702 | 7.390 | 1.00 | 28.84 | A | C | |
| ATOM | 1325 | C | ALA | A | 792 | 18.742 | 31.407 | 8.300 | 1.00 | 28.92 | A | C | |
| ATOM | 1326 | O | ALA | A | 792 | 19.724 | 30.850 | 8.790 | 1.00 | 29.89 | A | O | |
| ATOM | 1327 | N | LYS | A | 793 | 17.845 | 30.788 | 7.536 | 1.00 | 28.40 | A | N | |
| ATOM | 1328 | CA | LYS | A | 793 | 17.935 | 29.376 | 7.141 | 1.00 | 26.56 | A | C | |
| ATOM | 1329 | CB | LYS | A | 793 | 17.190 | 28.482 | 8.137 | 1.00 | 26.44 | A | C | |
| ATOM | 1330 | CG | LYS | A | 793 | 17.927 | 28.232 | 9.437 | 1.00 | 26.60 | A | C | |
| ATOM | 1331 | CD | LYS | A | 793 | 16.970 | 27.847 | 10.542 | 1.00 | 24.41 | A | C | |
| ATOM | 1332 | CE | LYS | A | 793 | 17.671 | 26.936 | 11.501 | 1.00 | 23.61 | A | C | |
| ATOM | 1333 | NZ | LYS | A | 793 | 17.798 | 25.611 | 10.839 | 1.00 | 22.29 | A | N | |
| ATOM | 1334 | C | LYS | A | 793 | 17.241 | 29.237 | 5.796 | 1.00 | 25.29 | A | C | |
| ATOM | 1335 | O | LYS | A | 793 | 16.020 | 29.191 | 5.778 | 1.00 | 26.49 | A | O | |
| ATOM | 1336 | N | ILE | A | 794 | 17.970 | 29.190 | 4.680 | 1.00 | 24.36 | A | N | |
| ATOM | 1337 | CA | ILE | A | 794 | 17.293 | 29.026 | 3.382 | 1.00 | 23.29 | A | C | |
| ATOM | 1338 | CB | ILE | A | 794 | 18.249 | 29.072 | 2.178 | 1.00 | 24.59 | A | C | |
| ATOM | 1339 | CG2 | ILE | A | 794 | 18.220 | 30.467 | 1.503 | 1.00 | 21.76 | A | C | |
| ATOM | 1340 | CG1 | ILE | A | 794 | 19.629 | 25.589 | 2.619 | 1.00 | 25.81 | A | C | |
| ATOM | 1341 | CD1 | ILE | A | 794 | 20.557 | 28.266 | 1.447 | 1.00 | 27.91 | A | C | |
| ATOM | 1342 | C | ILE | A | 794 | 16.598 | 27.669 | 3.292 | 1.00 | 21.79 | A | C | |
| ATOM | 1343 | O | ILE | A | 794 | 16.986 | 26.704 | 3.948 | 1.00 | 18.28 | A | O | |
| ATOM | 1344 | N | GLY | A | 795 | 15.558 | 27.617 | 2.472 | 1.00 | 22.23 | A | N | |
| ATOM | 1345 | CA | GLY | A | 795 | 14.817 | 26.383 | 2.278 | 1.00 | 23.50 | A | C | |
| ATOM | 1346 | C | GLY | A | 795 | 13.954 | 26.536 | 1.045 | 1.00 | 23.16 | A | C | |
| ATOM | 1347 | O | GLY | A | 795 | 14.258 | 27.369 | 0.183 | 1.00 | 24.05 | A | O | |
| ATOM | 1348 | N | ASP | A | 796 | 12.900 | 25.736 | 0.935 | 1.00 | 23.18 | A | N | |
| ATOM | 1349 | CA | ASP | A | 796 | 11.988 | 25.842 | −0.207 | 1.00 | 24.74 | A | C | |
| ATOM | 1350 | CB | ASP | A | 796 | 12.292 | 24.770 | −1.260 | 1.00 | 21.99 | A | C | |
| ATOM | 1351 | CG | ASP | A | 796 | 11.274 | 24.750 | −2.402 | 1.00 | 20.56 | A | C | |
| ATOM | 1352 | OD1 | ASP | A | 796 | 10.490 | 25.711 | −2.554 | 1.00 | 20.63 | A | O | |
| ATOM | 1353 | OD2 | ASP | A | 796 | 11.260 | 23.762 | −3.168 | 1.00 | 20.59 | A | O | |
| ATOM | 1354 | C | ASP | A | 796 | 10.589 | 25.665 | 0.359 | 1.00 | 27.66 | A | C | |
| ATOM | 1355 | O | ASP | A | 796 | 10.238 | 24.606 | 0.883 | 1.00 | 28.74 | A | O | |
| ATOM | 1356 | N | PHE | A | 797 | 9.784 | 24.706 | 0.267 | 1.00 | 30.29 | A | N | |
| ATOM | 1357 | CA | PHE | A | 797 | 8.446 | 26.630 | 0.826 | 1.00 | 33.58 | A | C | |
| ATOM | 1358 | CB | PHE | A | 797 | 8.257 | 27.818 | 1.767 | 1.00 | 32.46 | A | C | |
| ATOM | 1359 | CG | PHE | A | 797 | 9.443 | 28.061 | 2.657 | 1.00 | 28.84 | A | C | |
| ATOM | 1360 | CD1 | PHE | A | 797 | 9.963 | 29.339 | 2.813 | 1.00 | 27.57 | A | C | |
| ATOM | 1361 | CD2 | PHE | A | 797 | 10.055 | 27.005 | 3.314 | 1.00 | 27.78 | A | C | |
| ATOM | 1362 | CE1 | PHE | A | 797 | 11.080 | 29.565 | 3.606 | 1.00 | 26.86 | A | C | |
| ATOM | 1363 | CE2 | PHE | A | 797 | 11.179 | 27.225 | 4.118 | 1.00 | 27.11 | A | C | |
| ATOM | 1364 | CZ | PHE | A | 797 | 11.689 | 28.508 | 4.260 | 1.00 | 25.82 | A | C | |
| ATOM | 1365 | C | PHE | A | 797 | 7.331 | 26.577 | −0.228 | 1.00 | 36.07 | A | C | |
| ATOM | 1366 | O | PHE | A | 797 | 6.195 | 26.984 | 0.032 | 1.00 | 36.94 | A | O | |
| ATOM | 1367 | N | GLY | A | 798 | 7.664 | 26.077 | −1.415 | 1.00 | 37.75 | A | N | |
| ATOM | 1368 | CA | GLY | A | 798 | 6.669 | 29.598 | −2.463 | 1.00 | 40.17 | A | C | |
| ATOM | 1369 | C | GLY | A | 798 | 5.345 | 25.454 | −1.908 | 1.00 | 41.72 | A | C | |
| ATOM | 1370 | O | GLY | A | 798 | 4.293 | 25.985 | −2.282 | 1.00 | 42.22 | A | O | |
| ATOM | 1371 | N | LEU | A | 799 | 5.383 | 24.453 | −1.017 | 1.00 | 41.92 | A | N | |
| ATOM | 1372 | CA | LEU | A | 799 | 4.149 | 23.909 | −0.430 | 1.00 | 42.62 | A | C | |
| ATOM | 1373 | CB | LEU | A | 799 | 4.432 | 22.869 | 0.650 | 1.00 | 40.37 | A | C | |
| ATOM | 1374 | CG | LEU | A | 799 | 4.572 | 21.393 | 0.310 | 1.00 | 38.80 | A | C | |
| ATOM | 1375 | OD1 | LEU | A | 799 | 4.529 | 20.641 | 1.618 | 1.00 | 36.55 | A | C | |
| ATOM | 1376 | CD2 | LEU | A | 799 | 3.471 | 20.919 | −0.626 | 1.00 | 37.18 | A | C | |
| ATOM | 1377 | C | LEU | A | 799 | 3.303 | 24.990 | 0.218 | 1.00 | 44.55 | A | C | |
| ATOM | 1378 | O | LEU | A | 799 | 2.094 | 25.071 | −0.021 | 1.00 | 46.72 | A | O | |
| ATOM | 1379 | N | ALA | A | 800 | 3.944 | 25.812 | 1.045 | 1.00 | 45.01 | A | N | |
| ATOM | 1380 | CA | ALA | A | 800 | 3.255 | 26.880 | 1.753 | 1.00 | 45.56 | A | C | |
| ATOM | 1381 | CB | ALA | A | 800 | 4.186 | 27.490 | 2.756 | 1.00 | 45.21 | A | C | |
| ATOM | 1382 | C | ALA | A | 800 | 2.662 | 27.967 | 0.844 | 1.00 | 46.08 | A | C | |
| ATOM | 1383 | O | ALA | A | 800 | 1.445 | 28.142 | 0.803 | 1.00 | 47.63 | A | O | |
| ATOM | 1384 | N | ARG | A | 801 | 3.507 | 28.697 | 0.122 | 1.00 | 45.88 | A | N | |
| ATOM | 1385 | CA | ARG | A | 801 | 3.025 | 29.757 | −0.766 | 1.00 | 44.83 | A | C | |
| ATOM | 1386 | CB | ARG | A | 801 | 4.184 | 30.379 | −1.554 | 1.00 | 45.47 | A | C | |
| ATOM | 1387 | CG | ARG | A | 801 | 5.452 | 30.641 | −0.764 | 1.00 | 49.11 | A | C | |
| ATOM | 1388 | CD | ARG | A | 801 | 5.326 | 31.841 | 0.156 | 1.00 | 52.38 | A | C | |
| ATOM | 1389 | NE | ARG | A | 801 | 6.572 | 32.087 | 0.883 | 1.00 | 54.60 | A | N | |
| ATOM | 1390 | CZ | ARG | A | 801 | 6.756 | 33.090 | 1.734 | 1.00 | 55.08 | A | C | |
| ATOM | 1391 | NH1 | ARG | A | 801 | 5.772 | 33.963 | 1.967 | 1.00 | 55.63 | A | N | |
| ATOM | 1392 | NH2 | ARG | A | 801 | 7.918 | 33.219 | 2.366 | 1.00 | 53.49 | A | N | |
| ATOM | 1393 | C | ARG | A | 801 | 2.031 | 29.189 | −1.775 | 1.00 | 44.25 | A | C | |
| ATOM | 1394 | O | ARG | A | 801 | 2.038 | 27.987 | −2.060 | 1.00 | 42.45 | A | O | |
| ATOM | 1395 | N | ASP | A | 802 | 1.173 | 30.059 | −2.308 | 1.00 | 44.21 | A | N | |
| ATOM | 1396 | CA | ASP | A | 802 | 0.219 | 29.652 | −3.330 | 1.00 | 44.56 | A | C | |
| ATOM | 1397 | CB | ASP | A | 802 | −1.169 | 30.201 | −3.065 | 1.00 | 43.94 | A | C | |
| ATOM | 1398 | CG | ASP | A | 802 | −2.104 | 29.937 | −4.224 | 1.00 | 44.76 | A | C | |
| ATOM | 1399 | OD1 | ASP | A | 802 | −1.951 | 28.863 | −4.856 | 1.00 | 45.50 | A | O | |
| ATOM | 1400 | OD2 | ASP | A | 802 | −2.980 | 30.780 | −4.508 | 1.00 | 44.99 | A | O | |
| ATOM | 1401 | C | ASP | A | 802 | 0.698 | 30.213 | −4.653 | 1.00 | 46.09 | A | C | |
| ATOM | 1402 | O | ASP | A | 802 | 0.381 | 31.353 | −4.996 | 1.00 | 46.29 | A | O | |
| ATOM | 1403 | N | ILE | A | 803 | 1.457 | 29.411 | −5.396 | 1.00 | 48.06 | A | N | |
| ATOM | 1404 | CA | ILE | A | 803 | 2.009 | 29.838 | −6.686 | 1.00 | 49.16 | A | C | |
| ATOM | 1405 | CB | ILE | A | 803 | 2.970 | 28.774 | −7.266 | 1.00 | 49.80 | A | C | |
| ATOM | 1406 | CG2 | ILE | A | 803 | 3.561 | 29.271 | −8.563 | 1.00 | 49.54 | A | C | |
| ATOM | 1407 | CG1 | ILE | A | 803 | 4.105 | 28.502 | −6.276 | 1.00 | 49.74 | A | C | |
| ATOM | 1408 | CD1 | ILE | A | 803 | 5.047 | 27.411 | −6.735 | 1.00 | 51.31 | A | C | |
| ATOM | 1409 | C | ILE | A | 803 | 0.950 | 30.154 | −7.734 | 1.00 | 48.78 | A | C | |
| ATOM | 1410 | O | ILE | A | 803 | 1.180 | 30.958 | −8.643 | 1.00 | 49.97 | A | O | |
| ATOM | 1411 | N | MET | A | 804 | −0.206 | 29.521 | −7.623 | 1.00 | 48.18 | A | N | |
| ATOM | 1412 | CA | MET | A | 804 | −1.264 | 29.789 | −8.579 | 1.00 | 48.57 | A | C | |
| ATOM | 1413 | CB | MET | A | 804 | −2.493 | 28.934 | −8.266 | 1.00 | 48.91 | A | C | |
| ATOM | 1414 | CG | MET | A | 804 | −2.329 | 27.453 | −8.566 | 1.00 | 49.47 | A | C | |
| ATOM | 1415 | SD | MET | A | 804 | −1.731 | 27.144 | −10.242 | 1.00 | 50.31 | A | S | |
| ATOM | 1416 | CE | MET | A | 804 | −2.882 | 28.193 | −11.264 | 1.00 | 48.86 | A | C | |
| ATOM | 1417 | C | MET | A | 804 | −1.675 | 31.268 | −8.593 | 1.00 | 48.66 | A | C | |
| ATOM | 1418 | O | MET | A | 804 | −2.235 | 31.751 | −9.587 | 1.00 | 49.24 | A | O | |
| ATOM | 1419 | N | ASN | A | 805 | −1.392 | 31.992 | −7.510 | 1.00 | 47.80 | A | N | |
| ATOM | 1420 | CA | ASN | A | 805 | −1.795 | 33.397 | −7.437 | 1.00 | 47.66 | A | C | |
| ATOM | 1421 | CB | ASN | A | 805 | −2.982 | 33.517 | −6.459 | 1.00 | 46.12 | A | C | |
| ATOM | 1422 | CG | ASN | A | 805 | −4.217 | 32.707 | −6.935 | 1.00 | 47.45 | A | C | |
| ATOM | 1423 | OD1 | ASN | A | 805 | −4.862 | 33.062 | −7.935 | 1.00 | 44.80 | A | O | |
| ATOM | 1424 | ND2 | ASN | A | 805 | −4.532 | 31.608 | −6.227 | 1.00 | 45.99 | A | N | |
| ATOM | 1425 | C | ASN | A | 805 | −0.681 | 34.401 | −7.095 | 1.00 | 47.57 | A | C | |
| ATOM | 1426 | O | ASN | A | 805 | −0.946 | 35.576 | −6.820 | 1.00 | 47.42 | A | O | |
| ATOM | 1427 | N | ASP | A | 806 | 0.569 | 33.946 | −7.136 | 1.00 | 47.31 | A | N | |
| ATOM | 1428 | CA | ASP | A | 806 | 1.699 | 34.822 | −6.832 | 1.00 | 46.51 | A | C | |
| ATOM | 1429 | CB | ASP | A | 806 | 2.795 | 34.045 | −6.106 | 1.00 | 44.80 | A | C | |
| ATOM | 1430 | CG | ASP | A | 806 | 3.923 | 34.938 | −5.638 | 1.00 | 44.35 | A | C | |
| ATOM | 1431 | OD1 | ASP | A | 806 | 4.218 | 35.920 | −6.349 | 1.00 | 45.00 | A | O | |
| ATOM | 1432 | OD2 | ASP | A | 806 | 4.524 | 34.662 | −4.573 | 1.00 | 42.69 | A | O | |
| ATOM | 1433 | C | ASP | A | 806 | 2.256 | 35.412 | −8.128 | 1.00 | 47.25 | A | C | |
| ATOM | 1434 | O | ASP | A | 806 | 2.858 | 34.698 | −8.943 | 1.00 | 48.15 | A | O | |
| ATOM | 1435 | N | SER | A | 807 | 2.058 | 36.716 | −8.309 | 1.00 | 46.99 | A | N | |
| ATOM | 1436 | CA | SER | A | 807 | 2.512 | 37.434 | −9.503 | 1.00 | 45.34 | A | C | |
| ATOM | 1437 | CB | SER | A | 807 | 2.095 | 38.902 | −9.403 | 1.00 | 46.69 | A | C | |
| ATOM | 1438 | OG | SER | A | 807 | 2.426 | 39.438 | −8.124 | 1.00 | 47.87 | A | O | |
| ATOM | 1439 | C | SER | A | 807 | 4.011 | 37.354 | −9.762 | 1.00 | 44.82 | A | C | |
| ATOM | 1440 | O | SER | A | 807 | 4.512 | 37.956 | −10.715 | 1.00 | 44.40 | A | C | |
| ATOM | 1441 | N | ASN | A | 808 | 4.739 | 36.631 | −8.917 | 1.00 | 44.38 | A | N | |
| ATOM | 1442 | CA | ASN | A | 808 | 6.176 | 36.495 | −9.123 | 1.00 | 44.20 | A | O | |
| ATOM | 1443 | CB | ASN | A | 808 | 6.901 | 36.382 | −7.794 | 1.00 | 42.14 | A | C | |
| ATOM | 1444 | CG | ASN | A | 808 | 6.764 | 37.625 | −6.976 | 1.00 | 41.22 | A | C | |
| ATOM | 1445 | OD1 | ASN | A | 808 | 5.926 | 37.699 | −6.077 | 1.00 | 40.16 | A | O | |
| ATOM | 1446 | ND2 | ASN | A | 808 | 7.572 | 38.634 | −7.295 | 1.00 | 40.32 | A | N | |
| ATOM | 1447 | C | ASN | A | 808 | 6.515 | 35.297 | −9.995 | 1.00 | 45.11 | A | C | |
| ATOM | 1448 | O | ASN | A | 808 | 7.596 | 35.241 | −10.596 | 1.00 | 45.69 | A | O | |
| ATOM | 1449 | N | TYR | A | 809 | 5.596 | 34.337 | −10.062 | 1.00 | 45.48 | A | N | |
| ATOM | 1450 | CA | TYR | A | 809 | 5.801 | 33.151 | −10.884 | 1.00 | 45.96 | A | C | |
| ATOM | 1451 | CB | TYR | A | 809 | 5.306 | 31.894 | −10.141 | 1.00 | 44.87 | A | C | |
| ATOM | 1452 | CG | TYR | A | 809 | 6.103 | 31.567 | −8.878 | 1.00 | 42.44 | A | C | |
| ATOM | 1453 | CD1 | TYR | A | 809 | 5.931 | 32.302 | −7.705 | 1.00 | 41.24 | A | C | |
| ATOM | 1454 | CE1 | TYR | A | 809 | 6.735 | 32.082 | −6.590 | 1.00 | 40.55 | A | C | |
| ATOM | 1455 | CD2 | TYR | A | 809 | 7.098 | 30.587 | −8.895 | 1.00 | 41.55 | A | C | |
| ATOM | 1456 | CE2 | TYR | A | 809 | 7.905 | 30.356 | −7.788 | 1.00 | 40.97 | A | C | |
| ATOM | 1457 | CZ | TYR | A | 809 | 7.724 | 31.113 | −6.641 | 1.00 | 41.56 | A | C | |
| ATOM | 1458 | OH | TYR | A | 809 | 8.573 | 30.935 | −5.569 | 1.00 | 41.77 | A | O | |
| ATOM | 1459 | C | TYR | A | 809 | 5.052 | 33.353 | −12.205 | 1.00 | 47.93 | A | C | |
| ATOM | 1460 | O | TYR | A | 809 | 3.816 | 33.310 | −12.254 | 1.00 | 47.87 | A | O | |
| ATOM | 1461 | N | ILE | A | 810 | 5.811 | 33.630 | −13.265 | 1.00 | 49.68 | A | N | |
| ATOM | 1462 | CA | ILE | A | 810 | 5.239 | 33.837 | −14.591 | 1.00 | 51.31 | A | C | |
| ATOM | 1463 | CB | ILE | A | 810 | 6.240 | 34.542 | −15.556 | 1.00 | 50.90 | A | C | |
| ATOM | 1464 | CG2 | ILE | A | 810 | 5.575 | 34.796 | −16.911 | 1.00 | 50.67 | A | C | |
| ATOM | 1465 | CG1 | ILE | A | 810 | 6.733 | 35.863 | −14.948 | 1.00 | 49.67 | A | C | |
| ATOM | 1466 | CD1 | ILE | A | 810 | 5.648 | 36.910 | −14.727 | 1.00 | 50.14 | A | C | |
| ATOM | 1467 | C | ILE | A | 810 | 4.965 | 32.428 | −15.090 | 1.00 | 53.20 | A | C | |
| ATOM | 1468 | O | ILE | A | 810 | 5.675 | 31.496 | −14.712 | 1.00 | 53.01 | A | O | |
| ATOM | 1469 | N | VAL | A | 811 | 3.943 | 32.267 | −15.925 | 1.00 | 55.80 | A | N | |
| ATOM | 1470 | CA | VAL | A | 811 | 3.589 | 30.941 | −16.421 | 1.00 | 58.00 | A | C | |
| ATOM | 1471 | CB | VAL | A | 811 | 2.107 | 30.854 | −16.838 | 1.00 | 57.90 | A | C | |
| ATOM | 1472 | CG1 | VAL | A | 811 | 1.757 | 29.406 | −17.135 | 1.00 | 57.27 | A | C | |
| ATOM | 1473 | CG2 | VAL | A | 811 | 1.206 | 31.416 | −15.742 | 1.00 | 57.65 | A | C | |
| ATOM | 1474 | C | VAL | A | 811 | 4.424 | 30.509 | −17.608 | 1.00 | 59.84 | A | C | |
| ATOM | 1475 | O | VAL | A | 811 | 4.745 | 31.318 | −18.488 | 1.00 | 59.79 | A | O | |
| ATOM | 1476 | N | LYS | A | 812 | 4.750 | 29.219 | −17.626 | 1.00 | 61.92 | A | N | |
| ATOM | 1477 | CA | LYS | A | 812 | 5.556 | 26.618 | −18.684 | 1.00 | 64.39 | A | C | |
| ATOM | 1478 | CB | LYS | A | 812 | 6.680 | 27.774 | −18.068 | 1.00 | 64.37 | A | C | |
| ATOM | 1479 | CG | LYS | A | 812 | 8.053 | 27.933 | −18.717 | 1.00 | 65.27 | A | C | |
| ATOM | 1480 | CD | LYS | A | 812 | 8.109 | 27.421 | −20.153 | 1.00 | 65.52 | A | C | |
| ATOM | 1481 | CE | LYS | A | 812 | 9.523 | 27.567 | −20.731 | 1.00 | 65.92 | A | C | |
| ATOM | 1482 | NZ | LYS | A | 812 | 9.622 | 27.067 | −22.138 | 1.00 | 66.18 | A | N | |
| ATOM | 1483 | C | LYS | A | 812 | 4.681 | 27.722 | −19.560 | 1.00 | 65.99 | A | C | |
| ATOM | 1484 | O | LYS | A | 812 | 5.193 | 26.963 | −20.383 | 1.00 | 66.62 | A | O | |
| ATOM | 1485 | N | GLY | A | 813 | 3.364 | 27.802 | −19.372 | 1.00 | 67.59 | A | N | |
| ATOM | 1486 | CA | GLY | A | 813 | 2.451 | 26.984 | −20.162 | 1.00 | 68.75 | A | C | |
| ATOM | 1487 | C | GLY | A | 813 | 1.173 | 26.586 | −19.436 | 1.00 | 69.34 | A | C | |
| ATOM | 1488 | O | GLY | A | 813 | 0.350 | 25.824 | −19.961 | 1.00 | 69.57 | A | O | |
| ATOM | 1489 | N | ALA | A | 815 | 4.920 | 25.771 | −16.664 | 1.00 | 47.05 | A | N | |
| ATOM | 1490 | CA | ALA | A | 815 | 4.859 | 25.737 | −15.208 | 1.00 | 46.22 | A | C | |
| ATOM | 1491 | CB | ALA | A | 815 | 6.033 | 24.927 | −14.649 | 1.00 | 44.49 | A | C | |
| ATOM | 1492 | C | ALA | A | 815 | 4.867 | 27.150 | −14.619 | 1.00 | 46.57 | A | C | |
| ATOM | 1493 | O | ALA | A | 815 | 4.404 | 28.114 | −15.255 | 1.00 | 46.62 | A | O | |
| ATOM | 1494 | N | ARG | A | 816 | 5.402 | 27.259 | −13.403 | 1.00 | 46.06 | A | N | |
| ATOM | 1495 | CA | ARG | A | 816 | 5.481 | 28.524 | −12.686 | 1.00 | 44.06 | A | C | |
| ATOM | 1496 | CB | ARG | A | 816 | 4.564 | 28.460 | −11.471 | 1.00 | 44.35 | A | C | |
| ATOM | 1497 | CG | ARG | A | 816 | 3.120 | 28.148 | −11.803 | 1.00 | 44.43 | A | C | |
| ATOM | 1498 | CD | ARG | A | 816 | 2.407 | 29.380 | −12.326 | 1.00 | 46.99 | A | C | |
| ATOM | 1499 | NE | ARG | A | 816 | 1.056 | 29.067 | −12.776 | 1.00 | 48.19 | A | N | |
| ATOM | 1500 | CZ | ARG | A | 816 | 0.108 | 29.976 | −12.978 | 1.00 | 49.22 | A | C | |
| ATOM | 1501 | NH1 | ARG | A | 816 | 0.364 | 31.266 | −12.768 | 1.00 | 48.00 | A | N | |
| ATOM | 1502 | NH2 | ARG | A | 816 | −1.100 | 29.591 | −13.384 | 1.00 | 49.75 | A | N | |
| ATOM | 1503 | C | ARG | A | 816 | 6.920 | 28.772 | −12.242 | 1.00 | 43.22 | A | C | |
| ATOM | 1504 | O | ARG | A | 816 | 7.482 | 27.981 | −11.483 | 1.00 | 42.87 | A | O | |
| ATOM | 1505 | N | LEU | A | 817 | 7.507 | 29.872 | −12.711 | 1.00 | 42.48 | A | N | |
| ATOM | 1506 | CA | LEU | A | 817 | 8.887 | 30.213 | −12.373 | 1.00 | 41.35 | A | C | |
| ATOM | 1507 | CB | LEU | A | 817 | 9.788 | 29.978 | −13.586 | 1.00 | 40.61 | A | C | |
| ATOM | 1508 | CG | LEU | A | 817 | 9.796 | 28.614 | −14.284 | 1.00 | 40.08 | A | C | |
| ATOM | 1509 | CD1 | LEU | A | 817 | 10.894 | 28.607 | −15.357 | 1.00 | 39.23 | A | C | |
| ATOM | 1510 | CD2 | LEU | A | 817 | 10.050 | 27.504 | −13.273 | 1.00 | 40.83 | A | C | |
| ATOM | 1511 | C | LEU | A | 817 | 9.062 | 31.669 | −11.905 | 1.00 | 42.22 | A | C | |
| ATOM | 1512 | O | LEU | A | 817 | 8.431 | 32.588 | −12.443 | 1.00 | 43.26 | A | O | |
| ATOM | 1513 | N | PRO | A | 818 | 9.927 | 31.898 | −10.896 | 1.00 | 41.77 | A | N | |
| ATOM | 1514 | CD | PRO | A | 818 | 10.786 | 30.919 | −10.198 | 1.00 | 41.58 | A | C | |
| ATOM | 1515 | CA | PRO | A | 818 | 10.167 | 33.249 | −10.385 | 1.00 | 40.86 | A | C | |
| ATOM | 1516 | CB | PRO | A | 818 | 10.845 | 32.983 | −9.050 | 1.00 | 40.11 | A | C | |
| ATOM | 1517 | CG | PRO | A | 818 | 11.711 | 31.803 | −9.377 | 1.00 | 40.45 | A | C | |
| ATOM | 1518 | C | PRO | A | 818 | 11.103 | 33.906 | −11.381 | 1.00 | 40.85 | A | C | |
| ATOM | 1519 | O | PRO | A | 818 | 12.308 | 33.994 | −11.145 | 1.00 | 42.26 | A | O | |
| ATOM | 1520 | N | VAL | A | 819 | 10.551 | 34.363 | −12.498 | 1.00 | 40.30 | A | N | |
| ATOM | 1521 | CA | VAL | A | 819 | 11.369 | 34.958 | −13.548 | 1.00 | 39.24 | A | C | |
| ATOM | 1522 | CB | VAL | A | 819 | 10.506 | 35.417 | −14.736 | 1.00 | 39.81 | A | C | |
| ATOM | 1523 | CG1 | VAL | A | 819 | 11.415 | 35.814 | −15.915 | 1.00 | 38.99 | A | C | |
| ATOM | 1524 | CG2 | VAL | A | 819 | 9.554 | 34.303 | −15.138 | 1.00 | 38.68 | A | C | |
| ATOM | 1525 | C | VAL | A | 819 | 12.293 | 36.106 | −13.169 | 1.00 | 38.75 | A | C | |
| ATOM | 1526 | O | VAL | A | 819 | 13.3963 | 36.193 | −13.698 | 1.00 | 40.19 | A | O | |
| ATOM | 1527 | N | LYS | A | 820 | 11.865 | 36.994 | −12.275 | 1.00 | 38.33 | A | N | |
| ATOM | 1528 | CA | LYS | A | 820 | 12.718 | 38.123 | −11.893 | 1.00 | 36.81 | A | C | |
| ATOM | 1529 | CB | LYS | A | 820 | 11.898 | 39.173 | −11.148 | 1.00 | 35.36 | A | C | |
| ATOM | 1530 | CG | LYS | A | 820 | 10.915 | 39.904 | −12.036 | 1.00 | 34.95 | A | C | |
| ATOM | 1531 | CD | LYS | A | 820 | 10.074 | 40.898 | −11.257 | 1.00 | 36.22 | A | C | |
| ATOM | 1532 | CE | LYS | A | 820 | 9.275 | 41.793 | −12.203 | 1.00 | 37.60 | A | C | |
| ATOM | 1533 | NZ | LYS | A | 820 | 8.466 | 42.851 | −11.519 | 1.00 | 38.11 | A | N | |
| ATOM | 1534 | C | LYS | A | 820 | 13.942 | 37.728 | −11.060 | 1.00 | 37.42 | A | C | |
| ATOM | 1535 | O | LYS | A | 820 | 14.803 | 38.568 | −10.777 | 1.00 | 38.46 | A | O | |
| ATOM | 1536 | N | TRP | A | 821 | 14.027 | 36.453 | −10.680 | 1.00 | 37.35 | A | N | |
| ATOM | 1537 | CA | TRP | A | 821 | 15.151 | 35.942 | −9.887 | 1.00 | 37.18 | A | C | |
| ATOM | 1538 | CB | TRP | A | 821 | 14.636 | 35.116 | −8.690 | 1.00 | 34.50 | A | C | |
| ATOM | 1539 | CG | TRP | A | 821 | 14.216 | 35.951 | −7.510 | 1.00 | 30.89 | A | C | |
| ATOM | 1540 | CD2 | TRP | A | 821 | 12.960 | 36.626 | −7.330 | 1.00 | 29.37 | A | C | |
| ATOM | 1541 | CE2 | TRP | A | 821 | 13.055 | 37.373 | −6.132 | 1.00 | 28.80 | A | C | |
| ATOM | 1542 | CE3 | TRP | A | 821 | 11.766 | 36.677 | −8.067 | 1.00 | 28.13 | A | C | |
| ATOM | 1543 | CD1 | TRP | A | 821 | 14.991 | 36.297 | −6.444 | 1.00 | 29.50 | A | C | |
| ATOM | 1544 | NE1 | TRP | A | 821 | 14.304 | 37.154 | −5.614 | 1.00 | 28.41 | A | N | |
| ATOM | 1545 | CZ2 | TRP | A | 821 | 12.002 | 38.165 | −5.651 | 1.00 | 27.44 | A | C | |
| ATOM | 1546 | CZ3 | TRP | A | 821 | 10.712 | 37.471 | −7.588 | 1.00 | 27.54 | A | C | |
| ATOM | 1547 | CH2 | TRP | A | 821 | 10.844 | 38.203 | −6.391 | 1.00 | 28.55 | A | C | |
| ATOM | 1548 | C | TRP | A | 821 | 16.063 | 35.062 | −10.740 | 1.00 | 38.39 | A | C | |
| ATOM | 1549 | O | TRP | A | 821 | 17.273 | 34.963 | −10.501 | 1.00 | 38.45 | A | O | |
| ATOM | 1550 | N | MET | A | 822 | 15.473 | 34.428 | −11.744 | 1.00 | 39.53 | A | N | |
| ATOM | 1551 | CA | MET | A | 822 | 16.214 | 33.523 | −12.614 | 1.00 | 40.92 | A | C | |
| ATOM | 1552 | CB | MET | A | 822 | 15.250 | 32.805 | −13.555 | 1.00 | 40.44 | A | C | |
| ATOM | 1553 | CG | MET | A | 822 | 14.080 | 32.169 | −12.846 | 1.00 | 40.13 | A | C | |
| ATOM | 1554 | SD | MET | A | 822 | 13.276 | 31.016 | −13.946 | 1.00 | 40.12 | A | S | |
| ATOM | 1555 | CE | MET | A | 822 | 14.689 | 29.953 | −14.292 | 1.00 | 40.12 | A | C | |
| ATOM | 1556 | C | MET | A | 822 | 17.303 | 34.196 | −13.435 | 1.00 | 41.09 | A | C | |
| ATOM | 1557 | O | MET | A | 822 | 17.122 | 35.316 | −13.929 | 1.00 | 39.55 | A | O | |
| ATOM | 1558 | N | ALA | A | 823 | 18.426 | 33.488 | −13.576 | 1.00 | 41.48 | A | N | |
| ATOM | 1559 | CA | ALA | A | 823 | 19.560 | 33.976 | −14.346 | 1.00 | 42.84 | A | C | |
| ATOM | 1560 | CB | ALA | A | 823 | 20.728 | 33.040 | −14.196 | 1.00 | 42.51 | A | C | |
| ATOM | 1561 | C | ALA | A | 823 | 19.114 | 34.022 | −15.800 | 1.00 | 44.58 | A | C | |
| ATOM | 1562 | O | ALA | A | 823 | 18.021 | 33.553 | −16.128 | 1.00 | 46.00 | A | O | |
| ATOM | 1563 | N | PRO | A | 824 | 19.944 | 34.595 | −16.694 | 1.00 | 45.37 | A | N | |
| ATOM | 1564 | CD | PRO | A | 824 | 21.291 | 35.174 | −16.516 | 1.00 | 44.61 | A | C | |
| ATOM | 1565 | CA | PRO | A | 824 | 19.533 | 34.650 | −18.099 | 1.00 | 45.02 | A | C | |
| ATOM | 1566 | CB | PRO | A | 824 | 20.524 | 35.638 | −18.701 | 1.00 | 44.82 | A | C | |
| ATOM | 1567 | CG | PRO | A | 824 | 21.780 | 35.342 | −17.944 | 1.00 | 43.90 | A | C | |
| ATOM | 1568 | C | PRO | A | 824 | 19.672 | 33.251 | −18.671 | 1.00 | 45.28 | A | C | |
| ATOM | 1569 | O | PRO | A | 824 | 18.824 | 32.785 | −19.419 | 1.00 | 46.71 | A | O | |
| ATOM | 1570 | N | GLU | A | 825 | 20.745 | 32.580 | −18.278 | 1.00 | 45.14 | A | N | |
| ATOM | 1571 | CA | GLU | A | 825 | 21.017 | 31.232 | −18.726 | 1.00 | 45.11 | A | C | |
| ATOM | 1572 | CB | GLU | A | 825 | 22.281 | 30.702 | −18.036 | 1.00 | 45.92 | A | C | |
| ATOM | 1573 | CG | GLU | A | 825 | 22.074 | 30.353 | −16.559 | 1.00 | 47.24 | A | C | |
| ATOM | 1574 | CD | GLU | A | 825 | 23.307 | 30.602 | −15.702 | 1.00 | 48.26 | A | C | |
| ATOM | 1575 | OE1 | GLU | A | 825 | 23.659 | 31.789 | −15.495 | 1.00 | 47.23 | A | O | |
| ATOM | 1576 | OE2 | GLU | A | 825 | 23.923 | 29.609 | −15.234 | 1.00 | 49.60 | A | O | |
| ATOM | 1577 | C | GLU | A | 825 | 19.830 | 30.330 | −18.393 | 1.00 | 45.16 | A | C | |
| ATOM | 1578 | O | GLU | A | 825 | 19.592 | 29.345 | −19.090 | 1.00 | 46.79 | A | O | |
| ATOM | 1579 | N | SER | A | 826 | 19.086 | 30.657 | −17.338 | 1.00 | 44.06 | A | N | |
| ATOM | 1580 | CA | SER | A | 826 | 17.934 | 29.837 | −16.943 | 1.00 | 44.12 | A | C | |
| ATOM | 1581 | CB | SER | A | 826 | 17.698 | 29.925 | −15.434 | 1.00 | 42.91 | A | C | |
| ATOM | 1582 | OG | SER | A | 826 | 18.753 | 29.326 | −14.717 | 1.00 | 44.46 | A | O | |
| ATOM | 1583 | C | SER | A | 826 | 16.638 | 30.214 | −17.658 | 1.00 | 44.60 | A | C | |
| ATOM | 1584 | O | SER | A | 826 | 15.805 | 29.351 | −17.955 | 1.00 | 44.60 | A | O | |
| ATOM | 1585 | N | ILE | A | 827 | 16.455 | 31.502 | −17.915 | 1.00 | 44.67 | A | N | |
| ATOM | 1586 | CA | ILE | A | 827 | 15.247 | 31.954 | −18.588 | 1.00 | 46.62 | A | C | |
| ATOM | 1587 | CB | ILE | A | 827 | 15.121 | 33.491 | −18.531 | 1.00 | 47.35 | A | C | |
| ATOM | 1588 | CG2 | ILE | A | 827 | 13.728 | 33.917 | −19.007 | 1.00 | 45.99 | A | C | |
| ATOM | 1589 | CG1 | ILE | A | 827 | 15.400 | 33.989 | −17.107 | 1.00 | 47.61 | A | C | |
| ATOM | 1590 | CD1 | ILE | A | 827 | 15.586 | 35.509 | −17.008 | 1.00 | 47.72 | A | C | |
| ATOM | 1591 | C | ILE | A | 827 | 15.282 | 31.543 | −20.061 | 1.00 | 46.90 | A | C | |
| ATOM | 1592 | O | ILE | A | 827 | 14.308 | 31.020 | −20.607 | 1.00 | 47.05 | A | O | |
| ATOM | 1593 | N | PHE | A | 828 | 16.435 | 31.764 | −20.679 | 1.00 | 47.41 | A | N | |
| ATOM | 1594 | CA | PHE | A | 828 | 16.653 | 31.487 | −22.093 | 1.00 | 47.91 | A | C | |
| ATOM | 1595 | CB | PHE | A | 828 | 17.757 | 32.406 | −22.597 | 1.00 | 48.29 | A | C | |
| ATOM | 1596 | CG | PHE | A | 828 | 17.500 | 33.848 | −22.325 | 1.00 | 47.93 | A | C | |
| ATOM | 1597 | CD1 | PHE | A | 828 | 16.511 | 34.531 | −23.021 | 1.00 | 49.26 | A | C | |
| ATOM | 1598 | CD2 | PHE | A | 828 | 18.226 | 34.520 | −21.358 | 1.00 | 47.72 | A | C | |
| ATOM | 1599 | CE1 | PHE | A | 828 | 16.251 | 35.872 | −22.753 | 1.00 | 50.35 | A | C | |
| ATOM | 1600 | CE2 | PHE | A | 828 | 17.977 | 35.850 | −21.081 | 1.00 | 49.48 | A | C | |
| ATOM | 1601 | CZ | PHE | A | 828 | 16.985 | 36.534 | −21.781 | 1.00 | 49.28 | A | C | |
| ATOM | 1602 | C | PHE | A | 828 | 16.988 | 30.060 | −22.506 | 1.00 | 48.04 | A | C | |
| ATOM | 1603 | O | PHE | A | 828 | 16.429 | 29.558 | −23.482 | 1.00 | 47.41 | A | O | |
| ATOM | 1604 | N | ASP | A | 829 | 17.903 | 29.425 | −21.773 | 1.00 | 49.17 | A | N | |
| ATOM | 1605 | CA | ASP | A | 829 | 18.361 | 28.065 | −22.081 | 1.00 | 50.44 | A | C | |
| ATOM | 1606 | CB | ASP | A | 829 | 19.899 | 28.040 | −22.127 | 1.00 | 51.80 | A | C | |
| ATOM | 1607 | CG | ASP | A | 829 | 20.488 | 29.171 | −22.976 | 1.00 | 53.82 | A | C | |
| ATOM | 1608 | OD1 | ASP | A | 829 | 19.964 | 29.430 | −24.089 | 1.00 | 53.65 | A | O | |
| ATOM | 1609 | OD2 | ASP | A | 829 | 21.489 | 29.790 | −22.538 | 1.00 | 53.72 | A | O | |
| ATOM | 1610 | C | ASP | A | 829 | 17.888 | 26.950 | −21.135 | 1.00 | 50.70 | A | C | |
| ATOM | 1611 | O | ASP | A | 829 | 18.333 | 25.805 | −21.254 | 1.00 | 51.27 | A | O | |
| ATOM | 1612 | N | CYS | A | 830 | 16.991 | 27.264 | −20.206 | 1.00 | 51.18 | A | N | |
| ATOM | 1613 | CA | CYS | A | 830 | 16.521 | 26.257 | −19.253 | 1.00 | 51.26 | A | C | |
| ATOM | 1614 | CB | CYS | A | 830 | 15.559 | 25.286 | −19.939 | 1.00 | 50.99 | A | C | |
| ATOM | 1615 | SG | CYS | A | 830 | 14.034 | 26.085 | −20.528 | 1.00 | 53.22 | A | S | |
| ATOM | 1616 | C | CYS | A | 830 | 17.737 | 25.509 | −18.704 | 1.00 | 51.01 | A | C | |
| ATOM | 1617 | O | CYS | A | 830 | 17.956 | 24.335 | −19.002 | 1.00 | 51.56 | A | O | |
| ATOM | 1618 | N | VAL | A | 831 | 18.534 | 26.215 | −17.911 | 1.00 | 50.46 | A | N | |
| ATOM | 1619 | CA | VAL | A | 831 | 19.739 | 25.652 | −17.324 | 1.00 | 50.82 | A | C | |
| ATOM | 1620 | CB | VAL | A | 831 | 20.975 | 26.047 | −18.155 | 1.00 | 52.43 | A | C | |
| ATOM | 1621 | CG1 | VAL | A | 831 | 22.256 | 25.632 | −17.438 | 1.00 | 53.04 | A | C | |
| ATOM | 1622 | CG2 | VAL | A | 831 | 20.888 | 25.390 | −19.531 | 1.00 | 53.33 | A | C | |
| ATOM | 1623 | C | VAL | A | 831 | 19.902 | 26.144 | −15.889 | 1.00 | 50.25 | A | C | |
| ATOM | 1624 | O | VAL | A | 831 | 19.969 | 27.348 | −15.630 | 1.00 | 49.69 | A | O | |
| ATOM | 1625 | N | TYR | A | 832 | 19.971 | 25.194 | −14.963 | 1.00 | 49.59 | A | N | |
| ATOM | 1626 | CA | TYR | A | 832 | 20.088 | 25.498 | −13.549 | 1.00 | 48.73 | A | C | |
| ATOM | 1627 | CB | TYR | A | 832 | 18.849 | 25.012 | −12.822 | 1.00 | 50.01 | A | C | |
| ATOM | 1628 | CG | TYR | A | 832 | 17.577 | 25.649 | −13.303 | 1.00 | 50.90 | A | C | |
| ATOM | 1629 | CD1 | TYR | A | 832 | 17.149 | 25.488 | −14.614 | 1.00 | 51.52 | A | C | |
| ATOM | 1630 | CE1 | TYR | A | 832 | 15.948 | 26.042 | −15.048 | 1.00 | 53.03 | A | C | |
| ATOM | 1631 | CD2 | TYR | A | 832 | 16.777 | 26.386 | −12.430 | 1.00 | 52.51 | A | C | |
| ATOM | 1632 | CE2 | TYR | A | 832 | 15.572 | 26.942 | −12.848 | 1.00 | 53.68 | A | C | |
| ATOM | 1633 | CZ | TYR | A | 832 | 15.162 | 26.764 | −14.158 | 1.00 | 53.12 | A | C | |
| ATOM | 1634 | OH | TYR | A | 832 | 13.952 | 27.280 | −14.559 | 1.00 | 54.35 | A | O | |
| ATOM | 1635 | C | TYR | A | 832 | 21.306 | 24.864 | −12.914 | 1.00 | 48.22 | A | C | |
| ATOM | 1636 | O | TYR | A | 832 | 21.317 | 23.669 | −12.597 | 1.00 | 48.01 | A | O | |
| ATOM | 1637 | N | THR | A | 833 | 22.318 | 25.693 | 12.712 | 1.00 | 46.77 | A | N | |
| ATOM | 1638 | CA | THR | A | 833 | 23.579 | 25.284 | −12.113 | 1.00 | 45.84 | A | C | |
| ATOM | 1639 | CB | THR | A | 833 | 24.729 | 25.609 | −13.074 | 1.00 | 46.27 | A | C | |
| ATOM | 1640 | OG1 | THR | A | 833 | 25.479 | 26.732 | −12.583 | 1.00 | 47.62 | A | O | |
| ATOM | 1641 | CG2 | THR | A | 833 | 24.156 | 25.968 | −14.447 | 1.00 | 46.01 | A | C | |
| ATOM | 1642 | C | THR | A | 833 | 23.756 | 26.073 | −10.817 | 1.00 | 44.74 | A | C | |
| ATOM | 1643 | O | THR | A | 833 | 22.898 | 26.875 | −10.457 | 1.00 | 44.34 | A | O | |
| ATOM | 1644 | N | VAL | A | 834 | 24.859 | 25.848 | −10.113 | 1.00 | 44.12 | A | N | |
| ATOM | 1645 | CA | VAL | A | 834 | 25.108 | 26.585 | −8.878 | 1.00 | 43.23 | A | C | |
| ATOM | 1646 | CB | VAL | A | 834 | 26.343 | 26.007 | −8.109 | 1.00 | 41.84 | A | C | |
| ATOM | 1647 | CG1 | VAL | A | 834 | 26.759 | 26.928 | −6.980 | 1.00 | 41.68 | A | C | |
| ATOM | 1648 | CG2 | VAL | A | 834 | 25.998 | 24.670 | −7.532 | 1.00 | 40.77 | A | C | |
| ATOM | 1649 | C | VAL | A | 834 | 25.348 | 28.066 | −9.214 | 1.00 | 43.25 | A | C | |
| ATOM | 1650 | O | VAL | A | 834 | 25.020 | 28.963 | −8.423 | 1.00 | 42.09 | A | O | |
| ATOM | 1651 | N | GLN | A | 835 | 25.909 | 28.314 | −10.399 | 1.00 | 43.44 | A | N | |
| ATOM | 1652 | CA | GLN | A | 835 | 26.204 | 29.676 | −10.833 | 1.00 | 43.30 | A | C | |
| ATOM | 1653 | CB | GLN | A | 835 | 27.234 | 29.662 | −11.959 | 1.00 | 44.84 | A | C | |
| ATOM | 1654 | CG | GLN | A | 835 | 28.563 | 29.049 | −11.548 | 1.00 | 47.79 | A | C | |
| ATOM | 1655 | CD | GLN | A | 835 | 28.728 | 27.617 | −12.048 | 1.00 | 51.70 | A | C | |
| ATOM | 1656 | OE1 | GLN | A | 835 | 28.957 | 27.374 | −13.251 | 1.00 | 50.76 | A | O | |
| ATOM | 1657 | NE2 | GLN | A | 835 | 28.603 | 26.653 | −11.125 | 1.00 | 54.44 | A | N | |
| ATOM | 1658 | C | GLN | A | 835 | 24.939 | 30.393 | −11.274 | 1.00 | 42.24 | A | C | |
| ATOM | 1659 | O | GLN | A | 835 | 24.959 | 31.592 | −11.588 | 1.00 | 41.50 | A | O | |
| ATOM | 1660 | N | SER | A | 836 | 23.840 | 29.643 | −11.296 | 1.00 | 40.57 | A | N | |
| ATOM | 1661 | CA | SER | A | 836 | 22.543 | 30.192 | −11.646 | 1.00 | 39.60 | A | C | |
| ATOM | 1662 | CB | SER | A | 836 | 21.683 | 29.128 | −12.287 | 1.00 | 41.57 | A | C | |
| ATOM | 1663 | OG | SER | A | 836 | 22.483 | 27.997 | −12.546 | 1.00 | 46.23 | A | O | |
| ATOM | 1664 | C | SER | A | 836 | 21.933 | 30.588 | −10.319 | 1.00 | 38.43 | A | C | |
| ATOM | 1665 | O | SER | A | 836 | 21.189 | 31.575 | −10.228 | 1.00 | 37.93 | A | O | |
| ATOM | 1666 | N | ASP | A | 837 | 22.254 | 29.812 | −9.283 | 1.00 | 37.17 | A | N | |
| ATOM | 1667 | CA | ASP | A | 837 | 21.743 | 30.097 | −7.946 | 1.00 | 35.99 | A | C | |
| ATOM | 1668 | CB | ASP | A | 837 | 21.866 | 28.875 | −7.050 | 1.00 | 32.58 | A | C | |
| ATOM | 1669 | CG | ASP | A | 837 | 20.810 | 27.852 | −7.381 | 1.00 | 32.59 | A | C | |
| ATOM | 1670 | OD1 | ASP | A | 837 | 19.943 | 28.180 | −8.230 | 1.00 | 31.27 | A | O | |
| ATOM | 1671 | OD2 | ASP | A | 837 | 20.842 | 26.739 | −6.810 | 1.00 | 34.25 | A | O | |
| ATOM | 1672 | C | ASP | A | 837 | 22.454 | 31.280 | −7.329 | 1.00 | 36.00 | A | C | |
| ATOM | 1673 | O | ASP | A | 837 | 21.859 | 32.040 | −6.551 | 1.00 | 37.62 | A | O | |
| ATOM | 1674 | N | VAL | A | 838 | 23.725 | 31.444 | −7.681 | 1.00 | 34.50 | A | N | |
| ATOM | 1675 | CA | VAL | A | 838 | 24.476 | 32.569 | −7.177 | 1.00 | 32.49 | A | C | |
| ATOM | 1676 | CB | VAL | A | 838 | 25.900 | 32.533 | −7.655 | 1.00 | 31.06 | A | C | |
| ATOM | 1677 | CG1 | VAL | A | 838 | 26.601 | 33.788 | −7.213 | 1.00 | 32.20 | A | C | |
| ATOM | 1678 | CG2 | VAL | A | 838 | 26.587 | 31.316 | −7.095 | 1.00 | 31.15 | A | C | |
| ATOM | 1679 | C | VAL | A | 838 | 23.811 | 33.823 | −7.722 | 1.00 | 32.52 | A | C | |
| ATOM | 1680 | O | VAL | A | 838 | 23.615 | 34.797 | −6.995 | 1.00 | 33.25 | A | O | |
| ATOM | 1681 | N | TRP | A | 839 | 23.459 | 33.794 | −9.004 | 1.00 | 32.42 | A | N | |
| ATOM | 1682 | CA | TRP | A | 839 | 22.804 | 34.939 | −9.628 | 1.00 | 32.61 | A | C | |
| ATOM | 1683 | CB | TRP | A | 839 | 22.337 | 34.616 | −11.048 | 1.00 | 34.46 | A | C | |
| ATOM | 1684 | CG | TRP | A | 839 | 21.456 | 35.713 | −11.639 | 1.00 | 37.76 | A | C | |
| ATOM | 1685 | CD2 | TRP | A | 839 | 21.823 | 36.644 | −12.673 | 1.00 | 38.29 | A | C | |
| ATOM | 1686 | CE2 | TRP | A | 839 | 20.711 | 37.485 | −12.893 | 1.00 | 38.36 | A | C | |
| ATOM | 1687 | CE3 | TRP | A | 839 | 22.986 | 36.846 | −13.431 | 1.00 | 38.11 | A | C | |
| ATOM | 1688 | CD1 | TRP | A | 839 | 20.162 | 36.029 | −11.283 | 1.00 | 36.56 | A | C | |
| ATOM | 1689 | NE1 | TRP | A | 839 | 19.714 | 37.091 | −12.034 | 1.00 | 36.61 | A | N | |
| ATOM | 1690 | CZ2 | TRP | A | 839 | 20.731 | 38.515 | −13.851 | 1.00 | 40.38 | A | C | |
| ATOM | 1691 | CZ3 | TRP | A | 839 | 23.003 | 37.869 | −14.377 | 1.00 | 38.58 | A | C | |
| ATOM | 1692 | CH2 | TRP | A | 839 | 21.884 | 38.687 | −14.579 | 1.00 | 39.46 | A | C | |
| ATOM | 1693 | C | TRP | A | 839 | 21.586 | 35.356 | −8.831 | 1.00 | 31.93 | A | C | |
| ATOM | 1694 | O | TRP | A | 839 | 21.421 | 36.526 | −8.485 | 1.00 | 31.69 | A | O | |
| ATOM | 1695 | N | SER | A | 840 | 20.701 | 34.402 | −8.573 | 1.00 | 31.14 | A | N | |
| ATOM | 1696 | CA | SER | A | 840 | 19.504 | 34.739 | −7.828 | 1.00 | 29.25 | A | C | |
| ATOM | 1697 | CB | SER | A | 840 | 18.643 | 33.499 | −7.639 | 1.00 | 28.58 | A | C | |
| ATOM | 1698 | OG | SER | A | 840 | 17.997 | 33.182 | −8.861 | 1.00 | 25.79 | A | O | |
| ATOM | 1699 | C | SER | A | 840 | 19.943 | 35.314 | −6.494 | 1.00 | 27.95 | A | C | |
| ATOM | 1700 | O | SER | A | 840 | 19.485 | 36.381 | −6.071 | 1.00 | 27.37 | A | O | |
| ATOM | 1701 | N | TYR | A | 841 | 20.874 | 34.618 | −5.858 | 1.00 | 26.46 | A | N | |
| ATOM | 1702 | CA | TYR | A | 841 | 21.366 | 35.063 | −4.569 | 1.00 | 25.43 | A | C | |
| ATOM | 1703 | CB | TYR | A | 841 | 22.659 | 34.333 | −4.196 | 1.00 | 23.03 | A | C | |
| ATOM | 1704 | CG | TYR | A | 841 | 23.205 | 34.835 | −2.899 | 1.00 | 21.12 | A | C | |
| ATOM | 1705 | CD1 | TYR | A | 841 | 22.571 | 34.533 | −1.699 | 1.00 | 18.77 | A | C | |
| ATOM | 1706 | CE1 | TYR | A | 841 | 23.024 | 35.055 | −0.505 | 1.00 | 17.12 | A | C | |
| ATOM | 1707 | CD2 | TYR | A | 841 | 24.314 | 35.682 | −2.870 | 1.00 | 21.23 | A | C | |
| ATOM | 1708 | CE2 | TYR | A | 841 | 24.778 | 36.216 | −1.669 | 1.00 | 19.31 | A | C | |
| ATOM | 1709 | CZ | TYR | A | 841 | 24.126 | 35.884 | −0.499 | 1.00 | 18.51 | A | C | |
| ATOM | 1710 | OH | TYR | A | 841 | 24.633 | 36.314 | 0.692 | 1.00 | 20.49 | A | O | |
| ATOM | 1711 | C | TYR | A | 841 | 21.610 | 36.565 | −4.644 | 1.00 | 25.70 | A | C | |
| ATOM | 1712 | O | TYR | A | 841 | 21.177 | 37.316 | −3.788 | 1.00 | 27.50 | A | O | |
| ATOM | 1713 | N | GLY | A | 842 | 22.306 | 37.012 | −5.672 | 1.00 | 25.89 | A | N | |
| ATOM | 1714 | CA | GLY | A | 842 | 22.530 | 38.432 | −5.779 | 1.00 | 27.08 | A | C | |
| ATOM | 1715 | C | GLY | A | 842 | 21.186 | 39.143 | −5.714 | 1.00 | 28.76 | A | C | |
| ATOM | 1716 | O | GLY | A | 842 | 20.990 | 40.059 | −4.909 | 1.00 | 29.63 | A | O | |
| ATOM | 1717 | N | ILE | A | 843 | 20.248 | 38.723 | −6.559 | 1.00 | 29.17 | A | N | |
| ATOM | 1718 | CA | ILE | A | 843 | 18.923 | 39.341 | −6.571 | 1.00 | 29.04 | A | C | |
| ATOM | 1719 | CB | ILE | A | 843 | 17.922 | 38.519 | −7.424 | 1.00 | 29.83 | A | C | |
| ATOM | 1720 | CG2 | ILE | A | 843 | 16.558 | 39.213 | −7.435 | 1.00 | 29.22 | A | C | |
| ATOM | 1721 | CG1 | ILE | A | 843 | 18.473 | 38.346 | −8.844 | 1.00 | 30.14 | A | C | |
| ATOM | 1722 | CD1 | ILE | A | 843 | 18.849 | 39.681 | −9.546 | 1.00 | 30.86 | A | C | |
| ATOM | 1723 | C | ILE | A | 843 | 18.384 | 39.413 | −5.147 | 1.00 | 27.54 | A | C | |
| ATOM | 1724 | O | ILE | A | 843 | 17.593 | 40.303 | −4.814 | 1.00 | 27.05 | A | O | |
| ATOM | 1725 | N | LEU | A | 844 | 18.823 | 38.464 | −4.319 | 1.00 | 25.77 | A | N | |
| ATOM | 1726 | CA | LEU | A | 844 | 18.404 | 38.379 | −2.925 | 1.00 | 23.95 | A | C | |
| ATOM | 1727 | CB | LEU | A | 844 | 18.823 | 37.038 | −2.339 | 1.00 | 22.60 | A | C | |
| ATOM | 1728 | CG | LEU | A | 844 | 18.007 | 36.527 | −1.161 | 1.00 | 23.96 | A | C | |
| ATOM | 1729 | CD1 | LEU | A | 844 | 18.588 | 35.181 | −0.679 | 1.00 | 22.98 | A | C | |
| ATOM | 1730 | CD2 | LEU | A | 844 | 18.000 | 38.562 | −0.055 | 1.00 | 22.24 | A | C | |
| ATOM | 1731 | C | LEU | A | 844 | 19.042 | 39.520 | −2.138 | 1.00 | 23.82 | A | C | |
| ATOM | 1732 | O | LEU | A | 844 | 18.346 | 40.260 | −1.438 | 1.00 | 24.35 | A | O | |
| ATOM | 1733 | N | LEU | A | 845 | 20.362 | 39.667 | −2.253 | 1.00 | 23.97 | A | N | |
| ATOM | 1734 | CA | LEU | A | 845 | 21.068 | 40.747 | −1.563 | 1.00 | 24.96 | A | C | |
| ATOM | 1735 | CB | LEU | A | 845 | 22.493 | 40.912 | −2.092 | 1.00 | 24.83 | A | C | |
| ATOM | 1736 | CG | LEU | A | 845 | 23.617 | 40.104 | −1.455 | 1.00 | 26.19 | A | C | |
| ATOM | 1737 | CD1 | LEU | A | 845 | 24.951 | 40.580 | −2.047 | 1.00 | 25.66 | A | C | |
| ATOM | 1738 | CD2 | LEU | A | 845 | 23.587 | 40.280 | 0.064 | 1.00 | 25.43 | A | C | |
| ATOM | 1739 | C | LEU | A | 845 | 20.331 | 42.044 | −1.825 | 1.00 | 25.10 | A | C | |
| ATOM | 1740 | O | LEU | A | 845 | 20.213 | 42.886 | −0.943 | 1.00 | 26.26 | A | O | |
| ATOM | 1741 | N | TRP | A | 846 | 19.836 | 42.189 | −3.047 | 1.00 | 25.92 | A | N | |
| ATOM | 1742 | CA | TRP | A | 846 | 19.111 | 43.391 | −3.429 | 1.00 | 27.20 | A | C | |
| ATOM | 1743 | CB | TRP | A | 846 | 18.690 | 43.303 | −4.889 | 1.00 | 28.10 | A | C | |
| ATOM | 1744 | CG | TRP | A | 846 | 18.405 | 44.618 | −5.427 | 1.00 | 29.35 | A | C | |
| ATOM | 1745 | CD2 | TRP | A | 846 | 17.128 | 45.246 | −5.488 | 1.00 | 30.68 | A | C | |
| ATOM | 1746 | CE2 | TRP | A | 846 | 17.328 | 46.557 | −5.968 | 1.00 | 31.20 | A | C | |
| ATOM | 1747 | CE3 | TRP | A | 846 | 15.830 | 44.827 | −5.182 | 1.00 | 31.98 | A | C | |
| ATOM | 1748 | CD1 | TRP | A | 846 | 19.310 | 45.532 | −5.864 | 1.00 | 30.65 | A | C | |
| ATOM | 1749 | NE1 | TRP | A | 846 | 18.672 | 46.712 | −6.190 | 1.00 | 31.91 | A | N | |
| ATOM | 1750 | CZ2 | TRP | A | 846 | 16.278 | 47.455 | −6.151 | 1.00 | 31.91 | A | C | |
| ATOM | 1751 | CZ3 | TRP | A | 846 | 14.781 | 45.722 | −5.365 | 1.00 | 34.23 | A | C | |
| ATOM | 1752 | CH2 | TRP | A | 846 | 15.015 | 47.023 | −5.845 | 1.00 | 32.94 | A | C | |
| ATOM | 1753 | C | TRP | A | 846 | 17.878 | 43.598 | −2.540 | 1.00 | 28.07 | A | C | |
| ATOM | 1754 | O | TRP | A | 846 | 17.723 | 44.662 | −1.923 | 1.00 | 27.24 | A | O | |
| ATOM | 1755 | N | GLU | A | 847 | 17.015 | 42.580 | −2.472 | 1.00 | 27.74 | A | N | |
| ATOM | 1756 | CA | GLU | A | 847 | 15.815 | 42.634 | −1.642 | 1.00 | 28.78 | A | C | |
| ATOM | 1757 | CB | GLU | A | 847 | 15.130 | 41.273 | −1.579 | 1.00 | 28.44 | A | C | |
| ATOM | 1758 | CG | GLU | A | 847 | 14.909 | 40.591 | −2.895 | 1.00 | 28.83 | A | C | |
| ATOM | 1759 | CD | GLU | A | 847 | 14.358 | 39.191 | −2.709 | 1.00 | 30.52 | A | C | |
| ATOM | 1760 | OE1 | GLU | A | 847 | 13.155 | 39.058 | −2.390 | 1.00 | 29.79 | A | O | |
| ATOM | 1761 | OE2 | GLU | A | 847 | 15.141 | 38.219 | −2.871 | 1.00 | 32.84 | A | O | |
| ATOM | 1762 | C | GLU | A | 847 | 16.215 | 43.000 | −0.217 | 1.00 | 30.16 | A | C | |
| ATOM | 1763 | O | GLU | A | 847 | 15.569 | 43.817 | 0.438 | 1.00 | 32.31 | A | O | |
| ATOM | 1764 | N | ILE | A | 848 | 17.273 | 42.365 | 0.269 | 1.00 | 30.31 | A | N | |
| ATOM | 1765 | CA | ILE | A | 848 | 17.754 | 42.630 | 1.616 | 1.00 | 31.54 | A | C | |
| ATOM | 1766 | CB | ILE | A | 848 | 19.079 | 41.854 | 1.933 | 1.00 | 30.65 | A | C | |
| ATOM | 1767 | CG2 | ILE | A | 848 | 19.774 | 42.469 | 3.137 | 1.00 | 29.78 | A | C | |
| ATOM | 1768 | CG1 | ILE | A | 848 | 18.776 | 40.384 | 2.219 | 1.00 | 29.15 | A | C | |
| ATOM | 1769 | CD1 | ILE | A | 848 | 19.992 | 39.575 | 2.625 | 1.00 | 29.49 | A | C | |
| ATOM | 1770 | C | ILE | A | 848 | 18.011 | 44.109 | 1.868 | 1.00 | 32.93 | A | C | |
| ATOM | 1771 | O | ILE | A | 848 | 17.360 | 44.719 | 2.718 | 1.00 | 33.92 | A | O | |
| ATOM | 1772 | N | PHE | A | 849 | 18.951 | 44.691 | 1.128 | 1.00 | 33.60 | A | N | |
| ATOM | 1773 | CA | PHE | A | 849 | 19.299 | 46.087 | 1.360 | 1.00 | 34.71 | A | C | |
| ATOM | 1774 | CB | PHE | A | 849 | 20.688 | 46.353 | 0.800 | 1.00 | 34.23 | A | C | |
| ATOM | 1775 | CG | PHE | A | 849 | 21.771 | 45.709 | 1.605 | 1.00 | 35.50 | A | C | |
| ATOM | 1776 | CD1 | PHE | A | 849 | 22.219 | 46.304 | 2.787 | 1.00 | 35.86 | A | C | |
| ATOM | 1777 | CD2 | PHE | A | 849 | 22.301 | 44.478 | 1.226 | 1.00 | 36.01 | A | C | |
| ATOM | 1778 | CE1 | PHE | A | 849 | 23.176 | 45.683 | 3.582 | 1.00 | 35.75 | A | C | |
| ATOM | 1779 | CE2 | PHE | A | 849 | 23.264 | 43.843 | 2.013 | 1.00 | 36.59 | A | C | |
| ATOM | 1780 | CZ | PHE | A | 849 | 23.702 | 44.446 | 3.196 | 1.00 | 37.14 | A | C | |
| ATOM | 1781 | C | PHE | A | 849 | 18.311 | 47.137 | 0.895 | 1.00 | 34.75 | A | C | |
| ATOM | 1782 | O | PHE | A | 849 | 18.411 | 48.310 | 1.277 | 1.00 | 35.16 | A | O | |
| ATOM | 1783 | N | SER | A | 850 | 17.357 | 46.719 | 0.075 | 1.00 | 35.12 | A | N | |
| ATOM | 1784 | CA | SER | A | 850 | 16.342 | 47.633 | −0.416 | 1.00 | 35.03 | A | C | |
| ATOM | 1785 | CB | SER | A | 850 | 15.828 | 47.155 | −1.769 | 1.00 | 32.96 | A | C | |
| ATOM | 1786 | OG | SER | A | 850 | 15.498 | 45.783 | −1.698 | 1.00 | 33.19 | A | O | |
| ATOM | 1787 | C | SER | A | 850 | 15.231 | 47.605 | 0.630 | 1.00 | 36.05 | A | C | |
| ATOM | 1788 | O | SER | A | 850 | 14.315 | 48.437 | 0.623 | 1.00 | 36.81 | A | O | |
| ATOM | 1789 | N | LEU | A | 851 | 15.349 | 46.643 | 1.542 | 1.00 | 36.02 | A | N | |
| ATOM | 1790 | CA | LEU | A | 851 | 14.385 | 46.441 | 2.625 | 1.00 | 35.60 | A | C | |
| ATOM | 1791 | CB | LEU | A | 851 | 14.101 | 47.555 | 3.373 | 1.00 | 37.67 | A | C | |
| ATOM | 1792 | CG | LEU | A | 851 | 15.230 | 48.389. | 4.197 | 1.00 | 36.08 | A | C | |
| ATOM | 1793 | CD1 | LEU | A | 851 | 14.838 | 49.817 | 4.580 | 1.00 | 36.21 | A | C | |
| ATOM | 1794 | CD2 | LEU | A | 851 | 15.519 | 47.537 | 5.437 | 1.00 | 33.58 | A | C | |
| ATOM | 1795 | C | LEU | A | 851 | 13.077 | 45.860 | 2.119 | 1.00 | 34.18 | A | C | |
| ATOM | 1796 | O | LEU | A | 851 | 11.966 | 46.137 | 2.502 | 1.00 | 33.48 | A | O | |
| ATOM | 1797 | N | GLY | A | 852 | 13.181 | 44.863 | 1.247 | 1.00 | 33.26 | A | N | |
| ATOM | 1798 | CA | GLY | A | 852 | 11.991 | 44.215 | 0.739 | 1.00 | 34.41 | A | C | |
| ATOM | 1799 | C | GLY | A | 852 | 11.260 | 44.843 | −0.436. | 1.00 | 35.72 | A | C | |
| ATOM | 1800 | O | GLY | A | 852 | 10.046 | 44.661 | −0.585 | 1.00 | 37.01 | A | O | |
| ATOM | 1801 | N | LEU | A | 853 | 11.947 | 45.610 | −1.269 | 1.00 | 35.84 | A | N | |
| ATOM | 1802 | CA | LEU | A | 853 | 11.251 | 46.145 | −2.421 | 1.00 | 35.42 | A | C | |
| ATOM | 1803 | CB | LEU | A | 853 | 12.046 | 48.639 | −3.083 | 1.00 | 35.09 | A | C | |
| ATOM | 1804 | CG | LEU | A | 853 | 12.016 | 48.639 | −2.346 | 1.00 | 36.22 | A | C | |
| ATOM | 1805 | CD1 | LEU | A | 853 | 12.578 | 49.727 | −3.262 | 1.00 | 35.56 | A | C | |
| ATOM | 1806 | CD2 | LEU | A | 853 | 10.585 | 49.000 | −1.933 | 1.00 | 33.65 | A | C | |
| ATOM | 1807 | C | LEU | A | 853 | 11.150 | 44.927 | −3.333 | 1.00 | 36.14 | A | C | |
| ATOM | 1808 | O | LEU | A | 853 | 11.779 | 43.902 | −3.078 | 1.00 | 34.32 | A | O | |
| ATOM | 1809 | N | ASN | A | 854 | 10.332 | 45.006 | −4.367 | 1.00 | 37.36 | A | N | |
| ATOM | 1810 | CA | ASN | A | 854 | 10.207 | 43.882 | −5.270 | 1.00 | 39.40 | A | C | |
| ATOM | 1811 | CB | ASN | A | 854 | 8.778 | 43.822 | −5.818 | 1.00 | 40.56 | A | C | |
| ATOM | 1812 | CG | ASN | A | 854 | 8.611 | 42.810 | −6.944 | 1.00 | 41.40 | A | C | |
| ATOM | 1813 | OD1 | ASN | A | 854 | 8.783 | 43.143 | −8.123 | 1.00 | 40.39 | A | O | |
| ATOM | 1814 | ND2 | ASN | A | 854 | 8.277 | 41.565 | −6.584 | 1.00 | 41.80 | A | N | |
| ATOM | 1815 | C | ASN | A | 854 | 11.228 | 44.153 | −6.357 | 1.00 | 41.36 | A | C | |
| ATOM | 1816 | O | ASN | A | 854 | 11.360 | 45.290 | −6.823 | 1.00 | 44.25 | A | O | |
| ATOM | 1817 | N | PRO | A | 855 | 11.973 | 43.123 | −6.780 | 1.00 | 41.56 | A | N | |
| ATOM | 1818 | CD | PRO | A | 855 | 11.706 | 41.682 | −6.636 | 1.00 | 41.17 | A | C | |
| ATOM | 1819 | CA | PRO | A | 855 | 12.974 | 43.368 | −7.827 | 1.00 | 41.57 | A | C | |
| ATOM | 1820 | CB | PRO | A | 855 | 13.464 | 41.960 | −8.184 | 1.00 | 41.41 | A | C | |
| ATOM | 1821 | CG | PRO | A | 855 | 13.024 | 41.097 | −7.009 | 1.00 | 40.77 | A | C | |
| ATOM | 1822 | C | PRO | A | 855 | 12.354 | 44.068 | −9.042 | 1.00 | 41.80 | A | C | |
| ATOM | 1823 | O | PRO | A | 855 | 11.156 | 43.921 | −9.292 | 1.00 | 41.16 | A | O | |
| ATOM | 1824 | N | TYR | A | 856 | 13.168 | 44.826 | −9.778 | 1.00 | 42.44 | A | N | |
| ATOM | 1825 | CA | TYR | A | 856 | 12.702 | 45.509 | −10.983 | 1.00 | 42.53 | A | C | |
| ATOM | 1826 | CB | TYR | A | 856 | 12.589 | 44.485 | −12.122 | 1.00 | 41.46 | A | C | |
| ATOM | 1827 | CG | TYR | A | 856 | 13.868 | 43.708 | −12.378 | 1.00 | 40.50 | A | C | |
| ATOM | 1828 | CD1 | TYR | A | 856 | 15.010 | 44.347 | −12.860 | 1.00 | 41.18 | A | C | |
| ATOM | 1829 | CE1 | TYR | A | 856 | 16.213 | 43.653 | −13.055 | 1.00 | 39.36 | A | C | |
| ATOM | 1830 | CD2 | TYR | A | 856 | 13.954 | 42.344 | −12.100 | 1.00 | 40.90 | A | C | |
| ATOM | 1831 | CE2 | TYR | A | 856 | 15.159 | 41.634 | −12.295 | 1.00 | 39.00 | A | C | |
| ATOM | 1832 | CZ | TYR | A | 856 | 16.279 | 42.303 | −12.769 | 1.00 | 38.35 | A | C | |
| ATOM | 1833 | OH | TYR | A | 856 | 17.474 | 41.654 | −12.931 | 1.00 | 37.51 | A | O | |
| ATOM | 1834 | C | TYR | A | 856 | 11.340 | 46.179 | −10.740 | 1.00 | 44.72 | A | C | |
| ATOM | 1835 | O | TYR | A | 856 | 10.385 | 45.994 | −11.525 | 1.00 | 44.34 | A | O | |
| ATOM | 1836 | N | PRO | A | 857 | 11.234 | 46.978 | −9.656 | 1.00 | 45.22 | A | N | |
| ATOM | 1837 | CD | PRO | A | 857 | 12.359 | 47.599 | −8.934 | 1.00 | 45.05 | A | C | |
| ATOM | 1838 | CA | PRO | A | 857 | 9.979 | 47.662 | −9.342 | 1.00 | 45.77 | A | C | |
| ATOM | 1839 | CB | PRO | A | 857 | 10.389 | 48.615 | −8.207 | 1.00 | 44.86 | A | C | |
| ATOM | 1840 | CG | PRO | A | 857 | 11.793 | 48.966 | −8.591 | 1.00 | 45.19 | A | C | |
| ATOM | 1841 | C | PRO | A | 857 | 9.425 | 48.398 | −10.530 | 1.00 | 47.01 | A | C | |
| ATOM | 1842 | O | PRO | A | 857 | 10.175 | 49.009 | −11.284 | 1.00 | 48.01 | A | O | |
| ATOM | 1843 | N | GLY | A | 858 | 8.112 | 48.235 | −10.718 | 1.00 | 48.60 | A | N | |
| ATOM | 1844 | CA | GLY | A | 858 | 7.493 | 49.000 | −11.843 | 1.00 | 49.33 | A | C | |
| ATOM | 1845 | C | GLY | A | 858 | 7.651 | 48.211 | −13.121 | 1.00 | 49.93 | A | C | |
| ATOM | 1846 | O | GLY | A | 858 | 6.703 | 48.041 | −13.884 | 1.00 | 49.36 | A | O | |
| ATOM | 1847 | N | ILE | A | 859 | 8.861 | 47.725 | −13.362 | 1.00 | 51.82 | A | N | |
| ATOM | 1848 | CA | ILE | A | 859 | 9.116 | 49.946 | −14.564 | 1.00 | 52.74 | A | C | |
| ATOM | 1849 | CB | ILE | A | 859 | 10.600 | 46.510 | −14.648 | 1.00 | 51.90 | A | C | |
| ATOM | 1850 | CG2 | ILE | A | 859 | 10.820 | 45.612 | −15.875 | 1.00 | 49.64 | A | C | |
| ATOM | 1851 | CG1 | ILE | A | 859 | 11.490 | 47.757 | −14.682 | 1.00 | 51.23 | A | C | |
| ATOM | 1852 | CD1 | ILE | A | 859 | 12.971 | 47.468 | −14.810 | 1.00 | 52.25 | A | C | |
| ATOM | 1853 | C | ILE | A | 859 | 8.227 | 45.710 | −14.573 | 1.00 | 53.47 | A | C | |
| ATOM | 1854 | O | ILE | A | 859 | 8.190 | 44.952 | −13.062 | 1.00 | 54.33 | A | O | |
| ATOM | 1855 | N | LEU | A | 860 | 7.490 | 45.530 | −15.661 | 1.00 | 53.92 | A | N | |
| ATOM | 1856 | CA | LEU | A | 860 | 6.624 | 44.372 | −15.802 | 1.00 | 54.95 | A | C | |
| ATOM | 1857 | CB | LEU | A | 860 | 5.405 | 44.721 | −16.647 | 1.00 | 54.62 | A | C | |
| ATOM | 1858 | CG | LEU | A | 860 | 4.266 | 45.377 | −15.883 | 1.00 | 54.69 | A | C | |
| ATOM | 1859 | CD1 | LEU | A | 860 | 3.174 | 45.804 | −16.863 | 1.00 | 54.11 | A | C | |
| ATOM | 1860 | CD2 | LEU | A | 860 | 3.738 | 44.385 | −14.842 | 1.00 | 53.39 | A | C | |
| ATOM | 1861 | C | LEU | A | 860 | 7.414 | 43.269 | −16.485 | 1.00 | 55.92 | A | C | |
| ATOM | 1862 | O | LEU | A | 860 | 8.288 | 43.551 | −17.315 | 1.00 | 56.56 | A | O | |
| ATOM | 1863 | N | VAL | A | 861 | 7.116 | 42.018 | −16.137 | 1.00 | 56.40 | A | N | |
| ATOM | 1864 | CA | VAL | A | 861 | 7.810 | 40.886 | −16.741 | 1.00 | 56.75 | A | C | |
| ATOM | 1865 | CB | VAL | A | 861 | 7.794 | 39.648 | −15.837 | 1.00 | 57.11 | A | C | |
| ATOM | 1866 | CG1 | VAL | A | 861 | 8.291 | 38.433 | −16.614 | 1.00 | 57.72 | A | C | |
| ATOM | 1867 | CG2 | VAL | A | 861 | 8.672 | 39.878 | −14.636 | 1.00 | 57.58 | A | C | |
| ATOM | 1868 | C | VAL | A | 861 | 7.173 | 40.488 | −18.056 | 1.00 | 56.83 | A | C | |
| ATOM | 1869 | O | VAL | A | 861 | 5.994 | 40.146 | −18.096 | 1.00 | 56.20 | A | O | |
| ATOM | 1870 | N | ASN | A | 862 | 7.972 | 40.530 | −19.122 | 1.00 | 58.09 | A | N | |
| ATOM | 1871 | CA | ASN | A | 862 | 7.532 | 40.157 | −20.470 | 1.00 | 58.33 | A | C | |
| ATOM | 1872 | CB | ASN | A | 862 | 6.396 | 41.049 | −20.926 | 1.00 | 58.00 | A | C | |
| ATOM | 1873 | CG | ASN | A | 862 | 6.775 | 42.490 | −20.899 | 1.00 | 57.98 | A | C | |
| ATOM | 1874 | OD1 | ASN | A | 862 | 7.904 | 42.851 | −21.243 | 1.00 | 57.83 | A | O | |
| ATOM | 1875 | ND2 | ASN | A | 862 | 5.837 | 43.338 | −20.495 | 1.00 | 59.11 | A | N | |
| ATOM | 1876 | C | ASN | A | 862 | 8.675 | 40.272 | −21.477 | 1.00 | 58.22 | A | C | |
| ATOM | 1877 | O | ASN | A | 862 | 9.834 | 40.018 | −21.144 | 1.00 | 59.35 | A | O | |
| ATOM | 1878 | N | SER | A | 863 | 8.339 | 40.678 | −22.699 | 1.00 | 57.67 | A | N | |
| ATOM | 1879 | CA | SER | A | 863 | 9.314 | 40.820 | −23.786 | 1.00 | 57.58 | A | C | |
| ATOM | 1880 | CB | SER | A | 863 | 8.586 | 41.277 | −25.051 | 1.00 | 57.51 | A | C | |
| ATOM | 1881 | OG | SER | A | 863 | 7.434 | 40.475 | −25.272 | 1.00 | 57.98 | A | O | |
| ATOM | 1882 | C | SER | A | 863 | 10.474 | 41.778 | −23.484 | 1.00 | 56.90 | A | C | |
| ATOM | 1883 | O | SER | A | 863 | 11.650 | 41.384 | −23.503 | 1.00 | 56.18 | A | O | |
| ATOM | 1884 | N | LYS | A | 864 | 10.133 | 43.036 | −23.215 | 1.00 | 56.54 | A | N | |
| ATOM | 1885 | CA | LYS | A | 864 | 11.124 | 44.067 | −22.911 | 1.00 | 55.90 | A | C | |
| ATOM | 1886 | CB | LYS | A | 864 | 10.426 | 45.406 | −22.593 | 1.00 | 56.34 | A | C | |
| ATOM | 1887 | CG | LYS | A | 864 | 10.335 | 46.411 | −23.754 | 1.00 | 56.79 | A | C | |
| ATOM | 1888 | CD | LYS | A | 864 | 9.256 | 46.061 | −24.784 | 1.00 | 56.51 | A | C | |
| ATOM | 1889 | CE | LYS | A | 864 | 9.310 | 47.001 | −25.998 | 1.00 | 54.59 | A | C | |
| ATOM | 1890 | NZ | LYS | A | 864 | 8.320 | 46.616 | −27.037 | 1.00 | 52.64 | A | N | |
| ATOM | 1891 | C | LYS | A | 864 | 12.023 | 43.678 | −21.733 | 1.00 | 54.93 | A | C | |
| ATOM | 1892 | O | LYS | A | 864 | 13.225 | 43.953 | −21.737 | 1.00 | 54.82 | A | O | |
| ATOM | 1893 | N | PHE | A | 865 | 11.441 | 43.039 | −20.724 | 1.00 | 53.70 | A | N | |
| ATOM | 1894 | CA | PHE | A | 865 | 12.209 | 42.655 | −19.554 | 1.00 | 51.68 | A | C | |
| ATOM | 1895 | CB | PHE | A | 865 | 11.328 | 41.959 | −18.521 | 1.00 | 51.93 | A | C | |
| ATOM | 1896 | CG | PHE | A | 865 | 12.082 | 41.539 | −17.290 | 1.00 | 50.94 | A | C | |
| ATOM | 1897 | CD1 | PHE | A | 865 | 12.417 | 42.471 | −16.314 | 1.00 | 50.22 | A | C | |
| ATOM | 1898 | CD2 | PHE | A | 865 | 12.538 | 40.232 | −17.154 | 1.00 | 49.70 | A | C | |
| ATOM | 1899 | CE1 | PHE | A | 865 | 13.197 | 42.114 | −15.235 | 1.00 | 48.71 | A | C | |
| ATOM | 1900 | CE2 | PHE | A | 865 | 13.319 | 39.867 | −16.082 | 1.00 | 49.27 | A | C | |
| ATOM | 1901 | CZ | PHE | A | 865 | 13.650 | 40.812 | −15.120 | 1.00 | 49.95 | A | C | |
| ATOM | 1902 | C | PHE | A | 865 | 13.337 | 41.726 | −19.929 | 1.00 | 51.27 | A | C | |
| ATOM | 1903 | O | PHE | A | 865 | 14.509 | 42.032 | −19.684 | 1.00 | 50.46 | A | O | |
| ATOM | 1904 | N | TYR | A | 866 | 12.969 | 40.587 | −20.518 | 1.00 | 51.20 | A | N | |
| ATOM | 1905 | CA | TYR | A | 866 | 13.932 | 39.568 | −20.934 | 1.00 | 51.22 | A | C | |
| ATOM | 1906 | CB | TYR | A | 866 | 13.231 | 38.488 | −21.775 | 1.00 | 50.16 | A | C | |
| ATOM | 1907 | CG | TYR | A | 866 | 12.136 | 37.701 | −21.051 | 1.00 | 49.71 | A | C | |
| ATOM | 1908 | CD1 | TYR | A | 866 | 10.907 | 37.459 | −21.669 | 1.00 | 49.36 | A | C | |
| ATOM | 1909 | CE1 | TYR | A | 866 | 9.893 | 36.746. | −21.035 | 1.00 | 48.45 | A | C | |
| ATOM | 1910 | CD2 | TYR | A | 866 | 12.328 | 37.195 | −19.764 | 1.00 | 49.33 | A | C | |
| ATOM | 1911 | CE2 | TYR | A | 866 | 11.313 | 36.468 | −19.115 | 1.00 | 49.67 | A | C | |
| ATOM | 1912 | CZ | TYR | A | 866 | 10.095 | 36.251 | −19.763 | 1.00 | 49.61 | A | C | |
| ATOM | 1913 | OH | TYR | A | 866 | 9.078 | 35.539 | −19.152 | 1.00 | 48.47 | A | O | |
| ATOM | 1914 | C | TYR | A | 866 | 15.081 | 40.216 | −21.722 | 1.00 | 52.28 | A | C | |
| ATOM | 1915 | O | TYR | A | 866 | 16.265 | 39.999 | −21.410 | 1.00 | 51.80 | A | O | |
| ATOM | 1916 | N | LYS | A | 867 | 14.738 | 41.021 | −22.729 | 1.00 | 52.53 | A | N | |
| ATOM | 1917 | CA | LYS | A | 867 | 15.766 | 41.700 | −23.506 | 1.00 | 53.19 | A | C | |
| ATOM | 1918 | CB | LYS | A | 867 | 15.135 | 42.627 | −24.549 | 1.00 | 53.72 | A | C | |
| ATOM | 1919 | CG | LYS | A | 867 | 14.486 | 41.860 | −25.704 | 1.00 | 54.98 | A | C | |
| ATOM | 1920 | CD | LYS | A | 867 | 13.646 | 42.763 | −26.616 | 1.00 | 55.16 | A | C | |
| ATOM | 1921 | CE | LYS | A | 867 | 13.076 | 41.973 | −27.800 | 1.00 | 54.75 | A | C | |
| ATOM | 1922 | NZ | LYS | A | 867 | 11.947 | 42.678 | −28.500 | 1.00 | 53.29 | A | N | |
| ATOM | 1923 | C | LYS | A | 867 | 16.663 | 42.288 | −22.550 | 1.00 | 53.62 | A | C | |
| ATOM | 1924 | O | LYS | A | 867 | 17.885 | 42.388 | −22.622 | 1.00 | 54.34 | A | O | |
| ATOM | 1925 | N | LEU | A | 868 | 16.066 | 43.240 | −21.628 | 1.00 | 53.78 | A | N | |
| ATOM | 1926 | CA | LEU | A | 868 | 16.861 | 44.016 | −20.679 | 1.00 | 53.49 | A | C | |
| ATOM | 1927 | CB | LEU | A | 868 | 15.968 | 44.702 | −19.635 | 1.00 | 51.96 | A | C | |
| ATOM | 1928 | CG | LEU | A | 868 | 15.067 | 45.845 | −20.116 | 1.00 | 51.67 | A | C | |
| ATOM | 1929 | CD1 | LEU | A | 868 | 14.303 | 46.435 | −18.939 | 1.00 | 50.79 | A | C | |
| ATOM | 1930 | CD2 | LEU | A | 868 | 15.909 | 46.921 | −20.778 | 1.00 | 51.53 | A | C | |
| ATOM | 1931 | C | LEU | A | 868 | 17.911 | 43.166 | −19.962 | 1.00 | 54.47 | A | C | |
| ATOM | 1932 | O | LEU | A | 868 | 19.113 | 43.457 | −20.048 | 1.00 | 54.79 | A | O | |
| ATOM | 1933 | N | VAL | A | 869 | 17.476 | 42.108 | −19.276 | 1.00 | 54.34 | A | N | |
| ATOM | 1934 | CA | VAL | A | 869 | 18.428 | 41.283 | −18.533 | 1.00 | 54.52 | A | C | |
| ATOM | 1935 | CB | VAL | A | 869 | 17.718 | 40.172 | −17.727 | 1.00 | 54.55 | A | C | |
| ATOM | 1936 | CG1 | VAL | A | 869 | 16.729 | 40.798 | −16.746 | 1.00 | 53.88 | A | C | |
| ATOM | 1937 | CG2 | VAL | A | 869 | 17.016 | 39.209 | −18.663 | 1.00 | 55.04 | A | C | |
| ATOM | 1938 | C | VAL | A | 869 | 19.515 | 40.658 | −19.406 | 1.00 | 54.82 | A | C | |
| ATOM | 1939 | O | VAL | A | 869 | 20.700 | 40.730 | −19.058 | 1.00 | 54.33 | A | O | |
| ATOM | 1940 | N | LYS | A | 870 | 19.125 | 40.051 | −20.530 | 1.00 | 54.49 | A | N | |
| ATOM | 1941 | CA | LYS | A | 870 | 20.109 | 39.444 | −21.419 | 1.00 | 53.67 | A | C | |
| ATOM | 1942 | CB | LYS | A | 870 | 19.446 | 38.771 | −22.625 | 1.00 | 55.07 | A | C | |
| ATOM | 1943 | CG | LYS | A | 870 | 20.468 | 38.110 | −23.576 | 1.00 | 57.96 | A | C | |
| ATOM | 1944 | CD | LYS | A | 870 | 19.926 | 37.882 | −25.004 | 1.00 | 59.71 | A | C | |
| ATOM | 1945 | CE | LYS | A | 870 | 20.992 | 37.290 | −25.969 | 1.00 | 59.99 | A | C | |
| ATOM | 1946 | NZ | LYS | A | 870 | 22.201 | 38.158 | −26.197 | 1.00 | 57.97 | A | N | |
| ATOM | 1947 | C | LYS | A | 870 | 21.030 | 40.542 | −21.925 | 1.00 | 53.11 | A | C | |
| ATOM | 1948 | O | LYS | A | 870 | 22.244 | 40.358 | −22.010 | 1.00 | 53.00 | A | O | |
| ATOM | 1949 | N | ASP | A | 871 | 20.447 | 41.691 | −22.256 | 1.00 | 52.58 | A | N | |
| ATOM | 1950 | CA | ASP | A | 871 | 21.229 | 42.810 | −22.762 | 1.00 | 51.95 | A | C | |
| ATOM | 1951 | CB | ASP | A | 871 | 20.322 | 43.822 | −23.484 | 1.00 | 52.71 | A | C | |
| ATOM | 1952 | CG | ASP | A | 871 | 19.579 | 43.205 | −24.693 | 1.00 | 54.58 | A | C | |
| ATOM | 1953 | OD1 | ASP | A | 871 | 20.230 | 42.578 | −25.564 | 1.00 | 54.79 | A | O | |
| ATOM | 1954 | OD2 | ASP | A | 871 | 18.334 | 43.350 | −24.783 | 1.00 | 56.24 | A | O | |
| ATOM | 1955 | C | ASP | A | 871 | 22.046 | 43.499 | −21.668 | 1.00 | 51.49 | A | C | |
| ATOM | 1956 | O | ASP | A | 871 | 22.471 | 44.633 | −21.836 | 1.00 | 52.44 | A | O | |
| ATOM | 1957 | N | GLY | A | 872 | 22.252 | 42.815 | −20.541 | 1.00 | 51.03 | A | N | |
| ATOM | 1958 | CA | GLY | A | 872 | 23.068 | 43.359 | −19.458 | 1.00 | 48.81 | A | C | |
| ATOM | 1959 | C | GLY | A | 872 | 22.494 | 44.211 | −18.328 | 1.00 | 47.69 | A | C | |
| ATOM | 1960 | O | GLY | A | 872 | 23.170 | 44.423 | −17.322 | 1.00 | 47.38 | A | O | |
| ATOM | 1961 | N | TYR | A | 873 | 21.267 | 44.703 | −18.463 | 1.00 | 46.46 | A | N | |
| ATOM | 1962 | CA | TYR | A | 873 | 20.677 | 45.545 | −17.421 | 1.00 | 44.77 | A | C | |
| ATOM | 1963 | CB | TYR | A | 873 | 19.188 | 45.768 | −17.703 | 1.00 | 45.43 | A | C | |
| ATOM | 1964 | CG | TYR | A | 873 | 18.497 | 46.688 | −16.715 | 1.00 | 46.58 | A | C | |
| ATOM | 1965 | CD1 | TYR | A | 873 | 19.046 | 47.931 | −16.390 | 1.00 | 47.68 | A | C | |
| ATOM | 1966 | CE1 | TYR | A | 873 | 18.399 | 48.813 | −15.514 | 1.00 | 48.10 | A | C | |
| ATOM | 1967 | CD2 | TYR | A | 873 | 17.277 | 46.338 | −16.138 | 1.00 | 46.56 | A | C | |
| ATOM | 1968 | CE2 | TYR | A | 873 | 16.616 | 47.210 | −15.261 | 1.00 | 48.36 | A | C | |
| ATOM | 1969 | CZ | TYR | A | 873 | 17.184 | 48.451 | −14.953 | 1.00 | 48.99 | A | C | |
| ATOM | 1970 | OH | TYR | A | 873 | 16.537 | 49.330 | −14.096 | 1.00 | 49.14 | A | O | |
| ATOM | 1971 | C | TYR | A | 873 | 20.833 | 44.997 | −16.000 | 1.00 | 43.89 | A | C | |
| ATOM | 1972 | O | TYR | A | 873 | 20.675 | 43.797 | −15.764 | 1.00 | 42.83 | A | O | |
| ATOM | 1973 | N | GLN | A | 874 | 21.153 | 45.892 | −15.065 | 1.00 | 42.47 | A | N | |
| ATOM | 1974 | CA | GLN | A | 874 | 21.296 | 45.554 | −13.646 | 1.00 | 39.50 | A | C | |
| ATOM | 1975 | CB | GLN | A | 874 | 22.759 | 45.571 | −13.227 | 1.00 | 36.77 | A | C | |
| ATOM | 1976 | CG | GLN | A | 874 | 23.575 | 44.483 | −13.858 | 1.00 | 36.62 | A | C | |
| ATOM | 1977 | CD | GLN | A | 874 | 24.956 | 44.362 | −13.241 | 1.00 | 38.12 | A | C | |
| ATOM | 1978 | OE1 | GLN | A | 874 | 25.765 | 43.514 | −13.652 | 1.00 | 38.18 | A | O | |
| ATOM | 1979 | NE2 | GLN | A | 874 | 25.240 | 45.210 | −12.246 | 1.00 | 38.83 | A | N | |
| ATOM | 1980 | C | GLN | A | 874 | 20.531 | 46.601 | −12.842 | 1.00 | 39.65 | A | C | |
| ATOM | 1981 | O | GLN | A | 874 | 20.520 | 47.781 | −13.197 | 1.00 | 40.80 | A | O | |
| ATOM | 1982 | N | MET | A | 875 | 19.863 | 46.187 | −11.774 | 1.00 | 39.91 | A | N | |
| ATOM | 1983 | CA | MET | A | 875 | 19.128 | 47.160 | −10.977 | 1.00 | 38.05 | A | C | |
| ATOM | 1984 | CB | MET | A | 875 | 18.291 | 46.488 | −9.903 | 1.00 | 36.59 | A | C | |
| ATOM | 1985 | CG | MET | A | 875 | 17.058 | 45.821 | −10.426 | 1.00 | 35.06 | A | C | |
| ATOM | 1986 | SD | MET | A | 875 | 16.320 | 44.921 | −9.071 | 1.00 | 36.61 | A | S | |
| ATOM | 1987 | CE | MET | A | 875 | 17.741 | 43.782 | −8.662 | 1.00 | 34.79 | A | C | |
| ATOM | 1988 | C | MET | A | 875 | 20.114 | 48.078 | −10.309 | 1.00 | 38.08 | A | C | |
| ATOM | 1989 | O | MET | A | 875 | 21.250 | 47.685 | −10.004 | 1.00 | 35.99 | A | O | |
| ATOM | 1990 | N | ALA | A | 876 | 19.663 | 49.307 | −10.085 | 1.00 | 39.06 | A | N | |
| ATOM | 1991 | CA | ALA | A | 876 | 20.484 | 50.319 | −9.443 | 1.00 | 40.02 | A | C | |
| ATOM | 1992 | CB | ALA | A | 876 | 19.737 | 51.645 | −9.416 | 1.00 | 39.61 | A | C | |
| ATOM | 1993 | C | ALA | A | 876 | 20.795 | 49.867 | −8.024 | 1.00 | 40.10 | A | C | |
| ATOM | 1994 | O | ALA | A | 876 | 20.201 | 48.910 | −7.518 | 1.00 | 41.02 | A | O | |
| ATOM | 1995 | N | GLN | A | 877 | 21.732 | 50.545 | −7.383 | 1.00 | 40.19 | A | N | |
| ATOM | 1996 | CA | GLN | A | 877 | 22.064 | 50.202 | −6.012 | 1.00 | 40.94 | A | C | |
| ATOM | 1997 | CB | GLN | A | 877 | 23.221 | 51.070 | −5.514 | 1.00 | 40.44 | A | C | |
| ATOM | 1998 | CG | GLN | A | 877 | 23.805 | 50.612 | −4.201 | 1.00 | 38.88 | A | C | |
| ATOM | 1999 | CD | GLN | A | 877 | 25.003 | 51.443 | −3.827 | 1.00 | 40.07 | A | C | |
| ATOM | 2000 | OE1 | GLN | A | 877 | 25.837 | 51.785 | −4.681 | 1.00 | 36.60 | A | O | |
| ATOM | 2001 | NE2 | GLN | A | 877 | 25.108 | 51.775 | −2.543 | 1.00 | 41.58 | A | N | |
| ATOM | 2002 | C | GLN | A | 877 | 20.837 | 50.431 | −5.131 | 1.00 | 40.26 | A | C | |
| ATOM | 2003 | O | GLN | A | 877 | 20.039 | 51.339 | −5.391 | 1.00 | 41.16 | A | O | |
| ATOM | 2004 | N | PRO | A | 878 | 20.634 | 49.575 | −4.117 | 1.00 | 39.06 | A | N | |
| ATOM | 2005 | CD | PRO | A | 878 | 21.078 | 48.178 | −3.997 | 1.00 | 38.28 | A | C | |
| ATOM | 2006 | CA | PRO | A | 878 | 19.462 | 49.817 | −3.275 | 1.00 | 38.73 | A | C | |
| ATOM | 2007 | CB | PRO | A | 878 | 19.254 | 48.485 | −2.566 | 1.00 | 37.56 | A | C | |
| ATOM | 2008 | CG | PRO | A | 878 | 19.812 | 47.501 | −3.537 | 1.00 | 38.22 | A | C | |
| ATOM | 2009 | C | PRO | A | 878 | 19.859 | 50.919 | −2.311 | 1.00 | 40.02 | A | C | |
| ATOM | 2010 | O | PRO | A | 878 | 21.049 | 51.224 | −2.162 | 1.00 | 43.09 | A | O | |
| ATOM | 2011 | N | ALA | A | 879 | 18.874 | 51.506 | −1.647 | 1.00 | 40.03 | A | N | |
| ATOM | 2012 | CA | ALA | A | 879 | 19.112 | 52.595 | −0.698 | 1.00 | 38.55 | A | C | |
| ATOM | 2013 | CB | ALA | A | 879 | 17.759 | 53.093 | −0.132 | 1.00 | 39.34 | A | C | |
| ATOM | 2014 | C | ALA | A | 879 | 20.075 | 52.345 | 0.461 | 1.00 | 36.50 | A | C | |
| ATOM | 2015 | O | ALA | A | 879 | 20.874 | 53.220 | 0.780 | 1.00 | 34.67 | A | O | |
| ATOM | 2016 | N | PHE | A | 880 | 20.007 | 51.175 | 1.093 | 1.00 | 35.90 | A | N | |
| ATOM | 2017 | CA | PHE | A | 880 | 20.855 | 50.916 | 2.263 | 1.00 | 36.69 | A | C | |
| ATOM | 2018 | CB | PHE | A | 880 | 19.998 | 50.340 | 3.391 | 1.00 | 36.08 | A | C | |
| ATOM | 2019 | CG | PHE | A | 880 | 18.783 | 51.141 | 3.675 | 1.00 | 35.80 | A | C | |
| ATOM | 2020 | CD1 | PHE | A | 880 | 17.750 | 51.196 | 2.752 | 1.00 | 37.62 | A | C | |
| ATOM | 2021 | CD2 | PHE | A | 880 | 18.695 | 51.897 | 4.833 | 1.00 | 36.45 | A | C | |
| ATOM | 2022 | CE1 | PHE | A | 880 | 16.635 | 52.006 | 2.980 | 1.00 | 38.57 | A | C | |
| ATOM | 2023 | CE2 | PHE | A | 880 | 17.603 | 52.703 | 5.074 | 1.00 | 35.87 | A | C | |
| ATOM | 2024 | CZ | PHE | A | 880 | 16.566 | 52.760 | 4.144 | 1.00 | 37.72 | A | C | |
| ATOM | 2025 | C | PHE | A | 880 | 22.081 | 50.031 | 2.083 | 1.00 | 36.95 | A | C | |
| ATOM | 2026 | O | PHE | A | 880 | 22.791 | 49.720 | 3.058 | 1.00 | 35.55 | A | O | |
| ATOM | 2027 | N | ALA | A | 881 | 22.344 | 49.629 | 0.848 | 1.00 | 37.70 | A | N | |
| ATOM | 2028 | CA | ALA | A | 881 | 23.483 | 48.762 | 0.589 | 1.00 | 38.50 | A | C | |
| ATOM | 2029 | CB | ALA | A | 881 | 23.254 | 47.971 | −0.689 | 1.00 | 39.04 | A | C | |
| ATOM | 2030 | C | ALA | A | 881 | 24.774 | 49.540 | 0.477 | 1.00 | 38.99 | A | C | |
| ATOM | 2031 | O | ALA | A | 881 | 24.869 | 50.480 | −0.322 | 1.00 | 39.31 | A | O | |
| ATOM | 2032 | N | PRO | A | 882 | 25.782 | 49.179 | 1.294 | 1.00 | 39.35 | A | N | |
| ATOM | 2033 | CD | PRO | A | 882 | 25.758 | 48.198 | 2.394 | 1.00 | 39.77 | A | C | |
| ATOM | 2034 | CA | PRO | A | 882 | 27.075 | 49.877 | 1.232 | 1.00 | 39.44 | A | C | |
| ATOM | 2035 | CB | PRO | A | 882 | 27.925 | 49.139 | 2.267 | 1.00 | 38.67 | A | C | |
| ATOM | 2036 | CG | PRO | A | 882 | 26.918 | 48.666 | 3.265 | 1.00 | 39.70 | A | C | |
| ATOM | 2037 | C | PRO | A | 882 | 27.549 | 49.594 | −0.189 | 1.00 | 39.75 | A | C | |
| ATOM | 2038 | O | PRO | A | 882 | 27.027 | 48.676 | −0.830 | 1.00 | 40.47 | A | O | |
| ATOM | 2039 | N | LYS | A | 883 | 28.515 | 50.345 | −0.701 | 1.00 | 40.07 | A | N | |
| ATOM | 2040 | CA | LYS | A | 883 | 28.953 | 50.074 | −2.063 | 1.00 | 40.05 | A | C | |
| ATOM | 2041 | CB | LYS | A | 883 | 30.071 | 51.022 | −2.491 | 1.00 | 40.32 | A | C | |
| ATOM | 2042 | CG | LYS | A | 883 | 30.340 | 50.956 | −3.981 | 1.00 | 40.37 | A | C | |
| ATOM | 2043 | CD | LYS | A | 883 | 31.172 | 52.132 | −4.428 | 1.00 | 42.89 | A | C | |
| ATOM | 2044 | CE | LYS | A | 883 | 31.240 | 52.228 | −5.940 | 1.00 | 44.13 | A | C | |
| ATOM | 2045 | NZ | LYS | A | 883 | 32.135 | 53.346 | −6.359 | 1.00 | 44.31 | A | N | |
| ATOM | 2046 | C | LYS | A | 883 | 29.432 | 48.632 | −2.168 | 1.00 | 40.14 | A | C | |
| ATOM | 2047 | O | LYS | A | 883 | 28.884 | 47.845 | −2.945 | 1.00 | 41.79 | A | O | |
| ATOM | 2048 | N | ASN | A | 884 | 30.444 | 48.285 | −1.376 | 1.00 | 38.84 | A | N | |
| ATOM | 2049 | CA | ASN | A | 884 | 30.999 | 46.932 | −1.385 | 1.00 | 36.94 | A | C | |
| ATOM | 2050 | CB | ASN | A | 884 | 31.754 | 46.674 | −0.082 | 1.00 | 37.30 | A | C | |
| ATOM | 2051 | CG | ASN | A | 884 | 33.180 | 47.201 | −0.121 | 1.00 | 34.33 | A | C | |
| ATOM | 2052 | OD1 | ASN | A | 884 | 33.532 | 48.043 | −0.956 | 1.00 | 30.64 | A | O | |
| ATOM | 2053 | NE2 | ASN | A | 884 | 34.007 | 46.703 | 0.792 | 1.00 | 34.01 | A | N | |
| ATOM | 2054 | C | ASN | A | 884 | 29.937 | 45.863 | −1.591 | 1.00 | 35.29 | A | C | |
| ATOM | 2055 | O | ASN | A | 884 | 30.084 | 45.007 | −2.455 | 1.00 | 36.84 | A | O | |
| ATOM | 2056 | N | ILE | A | 885 | 28.870 | 45.906 | −0.806 | 1.00 | 33.33 | A | N | |
| ATOM | 2057 | CA | ILE | A | 885 | 27.807 | 44.925 | −0.970 | 1.00 | 31.56 | A | C | |
| ATOM | 2058 | CB | ILE | A | 885 | 26.680 | 45.118 | 0.081 | 1.00 | 30.08 | A | C | |
| ATOM | 2059 | CG2 | ILE | A | 885 | 25.360 | 44.577 | −0.448 | 1.00 | 26.71 | A | C | |
| ATOM | 2060 | CG1 | ILE | A | 885 | 27.067 | 44.400 | 1.376 | 1.00 | 30.16 | A | C | |
| ATOM | 2061 | CD1 | ILE | A | 885 | 28.559 | 44.562 | 1.750 | 1.00 | 31.47 | A | C | |
| ATOM | 2062 | C | ILE | A | 885 | 27.228 | 45.036 | −2.375 | 1.00 | 31.11 | A | C | |
| ATOM | 2063 | O | ILE | A | 885 | 26.948 | 44.025 | −3.020 | 1.00 | 31.24 | A | O | |
| ATOM | 2064 | N | TYR | A | 886 | 27.046 | 46.258 | −2.858 | 1.00 | 30.72 | A | N | |
| ATOM | 2065 | CA | TYR | A | 886 | 26.503 | 46.415 | −4.199 | 1.00 | 30.22 | A | C | |
| ATOM | 2066 | CB | TYR | A | 886 | 26.264 | 47.876 | −4.539 | 1.00 | 29.01 | A | C | |
| ATOM | 2067 | CG | TYR | A | 886 | 25.609 | 48.047 | −5.890 | 1.00 | 29.11 | A | C | |
| ATOM | 2068 | CD1 | TYR | A | 886 | 24.246 | 47.828 | −6.060 | 1.00 | 28.56 | A | C | |
| ATOM | 2069 | CE1 | TYR | A | 886 | 23.641 | 47.964 | −7.318 | 1.00 | 27.53 | A | C | |
| ATOM | 2070 | CD2 | TYR | A | 886 | 26.539 | 48.406 | −7.009 | 1.00 | 28.85 | A | C | |
| ATOM | 2071 | CE2 | TYR | A | 886 | 25.767 | 45.837 | −8.264 | 1.00 | 28.26 | A | C | |
| ATOM | 2072 | CZ | TYR | A | 886 | 24.406 | 48.311 | −8.411 | 1.00 | 27.60 | A | C | |
| ATOM | 2073 | OH | TYR | A | 886 | 23.834 | 48.385 | −9.668 | 1.00 | 27.32 | A | O | |
| ATOM | 2074 | C | TYR | A | 886 | 27.474 | 45.819 | −5.212 | 1.00 | 29.96 | A | C | |
| ATOM | 2075 | O | TYR | A | 886 | 27.066 | 45.355 | −6.281 | 1.00 | 30.42 | A | O | |
| ATOM | 2076 | N | SER | A | 887 | 28.762 | 45.838 | −4.886 | 1.00 | 28.70 | A | N | |
| ATOM | 2077 | CA | SER | A | 887 | 29.738 | 45.271 | −5.791 | 1.00 | 28.83 | A | C | |
| ATOM | 2078 | CB | SER | A | 887 | 31.152 | 45.447 | −5.256 | 1.00 | 28.50 | A | C | |
| ATOM | 2079 | OG | SER | A | 887 | 31.705 | 46.665 | −5.709 | 1.00 | 30.47 | A | O | |
| ATOM | 2080 | C | SER | A | 887 | 29.418 | 43.798 | −5.878 | 1.00 | 30.04 | A | C | |
| ATOM | 2081 | O | SER | A | 887 | 29.723 | 43.132 | −6.866 | 1.00 | 31.26 | A | O | |
| ATOM | 2082 | N | ILE | A | 888 | 28.787 | 43.288 | −4.830 | 1.00 | 30.43 | A | N | |
| ATOM | 2083 | CA | ILE | A | 888 | 38.431 | 41.882 | −4.791 | 1.00 | 29.38 | A | C | |
| ATOM | 2084 | CB | ILE | A | 888 | 28.158 | 41.410 | −3.331 | 1.00 | 28.67 | A | C | |
| ATOM | 2085 | CG2 | ILE | A | 888 | 27.778 | 39.941 | −3.327 | 1.00 | 29.47 | A | C | |
| ATOM | 2086 | CG1 | ILE | A | 888 | 29.412 | 41.624 | −2.475 | 1.00 | 28.02 | A | C | |
| ATOM | 2087 | CD1 | ILE | A | 888 | 29.475 | 40.765 | −1.232 | 1.00 | 28.31 | A | C | |
| ATOM | 2088 | C | ILE | A | 888 | 27.229 | 41.540 | −5.672 | 1.00 | 28.17 | A | C | |
| ATOM | 2089 | O | ILE | A | 888 | 27.268 | 40.566 | −6.419 | 1.00 | 30.39 | A | O | |
| ATOM | 2090 | N | MET | A | 889 | 26.162 | 42.138 | −5.603 | 1.00 | 26.74 | A | N | |
| ATOM | 2091 | CA | MET | A | 889 | 25.013 | 41.983 | −6.426 | 1.00 | 27.93 | A | C | |
| ATOM | 2092 | CB | MET | A | 889 | 23.919 | 43.034 | −6.286 | 1.00 | 26.87 | A | C | |
| ATOM | 2093 | CG | MET | A | 889 | 23.202 | 42.977 | −4.959 | 1.00 | 26.99 | A | C | |
| ATOM | 2094 | SD | MET | A | 889 | 22.300 | 44.489 | −4.574 | 1.00 | 25.48 | A | S | |
| ATOM | 2095 | CE | MET | A | 889 | 23.425 | 45.274 | −3.406 | 1.00 | 25.48 | A | C | |
| ATOM | 2096 | C | MET | A | 889 | 25.507 | 41.940 | −7.851 | 1.00 | 29.91 | A | C | |
| ATOM | 2097 | O | MET | A | 889 | 25.178 | 41.027 | −8.621 | 1.00 | 31.75 | A | O | |
| ATOM | 2098 | N | GLN | A | 890 | 26.336 | 42.925 | −8.176 | 1.00 | 30.71 | A | N | |
| ATOM | 2099 | CA | GLN | A | 890 | 26.898 | 43.058 | −9.503 | 1.00 | 31.31 | A | C | |
| ATOM | 2100 | CB | GLN | A | 890 | 27.861 | 44.236 | −9.522 | 1.00 | 32.84 | A | C | |
| ATOM | 2101 | CG | GLN | A | 890 | 27.196 | 45.516 | −9.059 | 1.00 | 32.34 | A | C | |
| ATOM | 2102 | CD | GLN | A | 890 | 27.700 | 46.713 | −9.802 | 1.00 | 32.17 | A | C | |
| ATOM | 2103 | OE1 | GLN | A | 890 | 28.833 | 47.151 | −9.594 | 1.00 | 32.52 | A | O | |
| ATOM | 2104 | NE2 | GLN | A | 890 | 26.864 | 47.254 | −10.694 | 1.00 | 32.54 | A | N | |
| ATOM | 2105 | C | GLN | A | 890 | 27.596 | 41.794 | −9.959 | 1.00 | 32.22 | A | C | |
| ATOM | 2106 | O | GLN | A | 890 | 27.170 | 41.191 | −10.947 | 1.00 | 33.98 | A | O | |
| ATOM | 2107 | N | ALA | A | 891 | 28.651 | 41.395 | −9.242 | 1.00 | 31.70 | A | N | |
| ATOM | 2108 | CA | ALA | A | 891 | 29.424 | 40.182 | −9.551 | 1.00 | 31.40 | A | C | |
| ATOM | 2109 | CB | ALA | A | 891 | 30.423 | 39.894 | −8.429 | 1.00 | 30.54 | A | C | |
| ATOM | 2110 | C | ALA | A | 891 | 28.500 | 38.977 | −9.724 | 1.00 | 32.62 | A | C | |
| ATOM | 2111 | O | ALA | A | 891 | 28.688 | 38.136 | −10.620 | 1.00 | 32.37 | A | O | |
| ATOM | 2112 | N | CYS | A | 892 | 27.502 | 38.880 | −8.852 | 1.00 | 32.29 | A | N | |
| ATOM | 2113 | CA | CYS | A | 892 | 26.564 | 37.775 | −8.951 | 1.00 | 31.85 | A | C | |
| ATOM | 2114 | CB | CYS | A | 892 | 25.541 | 37.836 | −7.823 | 1.00 | 28.88 | A | C | |
| ATOM | 2115 | SG | CYS | A | 892 | 26.292 | 37.430 | −6.235 | 1.00 | 23.34 | A | S | |
| ATOM | 2116 | C | CYS | A | 892 | 25.856 | 37.853 | −10.282 | 1.00 | 33.34 | A | C | |
| ATOM | 2117 | O | CYS | A | 892 | 25.528 | 36.826 | −10.882 | 1.00 | 35.16 | A | O | |
| ATOM | 2118 | N | TRP | A | 893 | 25.666 | 39.080 | −10.760 | 1.00 | 33.94 | A | N | |
| ATOM | 2119 | CA | TRP | A | 893 | 24.949 | 39.323 | −12.004 | 1.00 | 33.40 | A | C | |
| ATOM | 2120 | CB | TRP | A | 893 | 24.177 | 40.642 | −11.865 | 1.00 | 33.62 | A | C | |
| ATOM | 2121 | CG | TRP | A | 893 | 23.203 | 40.625 | −10.682 | 1.00 | 32.02 | A | C | |
| ATOM | 2122 | CD2 | TRP | A | 893 | 22.658 | 41.759 | −9.995 | 1.00 | 30.98 | A | C | |
| ATOM | 2123 | CE2 | TRP | A | 893 | 21.748 | 41.268 | −9.027 | 1.00 | 30.36 | A | C | |
| ATOM | 2124 | CE3 | TRP | A | 893 | 22.845 | 43.139 | −10.105 | 1.00 | 29.71 | A | C | |
| ATOM | 2125 | CD1 | TRP | A | 893 | 22.621 | 39.524 | −10.112 | 1.00 | 30.99 | A | C | |
| ATOM | 2126 | NE1 | TRP | A | 893 | 21.745 | 39.903 | −9.119 | 1.00 | 29.45 | A | N | |
| ATOM | 2127 | CZ2 | TRP | A | 893 | 21.031 | 42.107 | −8.182 | 1.00 | 31.14 | A | C | |
| ATOM | 2128 | CZ3 | TRP | A | 893 | 22.131 | 43.976 | −9.262 | 1.00 | 29.35 | A | C | |
| ATOM | 2129 | CH2 | TRP | A | 893 | 21.235 | 43.460 | −8.314 | 1.00 | 30.37 | A | C | |
| ATOM | 2130 | C | TRP | A | 893 | 25.750 | 39.293 | −13.305 | 1.00 | 32.80 | A | C | |
| ATOM | 2131 | O | TRP | A | 893 | 25.241 | 39.670 | −14.366 | 1.00 | 33.26 | A | O | |
| ATOM | 2132 | N | ALA | A | 894 | 26.992 | 38.833 | −13.240 | 1.00 | 31.92 | A | N | |
| ATOM | 2133 | CA | ALA | A | 894 | 27.809 | 38.744 | −14.447 | 1.00 | 32.67 | A | C | |
| ATOM | 2134 | CB | ALA | A | 894 | 29.161 | 38.152 | −14.112 | 1.00 | 32.52 | A | C | |
| ATOM | 2135 | C | ALA | A | 894 | 27.098 | 37.848 | −15.455 | 1.00 | 34.21 | A | C | |
| ATOM | 2136 | O | ALA | A | 894 | 26.756 | 36.703 | −15.137 | 1.00 | 34.47 | A | O | |
| ATOM | 2137 | N | LEU | A | 895 | 26.879 | 38.353 | −16.668 | 1.00 | 37.08 | A | N | |
| ATOM | 2138 | CA | LEU | A | 895 | 26.198 | 35.573 | −17.715 | 1.00 | 37.52 | A | C | |
| ATOM | 2139 | CB | LEU | A | 895 | 26.194 | 38.327 | −19.049 | 1.00 | 37.41 | A | C | |
| ATOM | 2140 | CG | LEU | A | 895 | 25.198 | 39.479 | −19.219 | 1.00 | 37.31 | A | C | |
| ATOM | 2141 | CD1 | LEU | A | 895 | 25.167 | 39.871 | −20.695 | 1.00 | 38.63 | A | C | |
| ATOM | 2142 | CD2 | LEU | A | 895 | 23.805 | 39.067 | −18.767 | 1.00 | 35.68 | A | C | |
| ATOM | 2143 | C | LEU | A | 895 | 26.780 | 36.179 | −17.937 | 1.00 | 37.71 | A | C | |
| ATOM | 2144 | O | LEU | A | 895 | 26.025 | 35.205 | −18.074 | 1.00 | 37.49 | A | O | |
| ATOM | 2145 | N | GLU | A | 896 | 28.106 | 36.059 | −17.985 | 1.00 | 37.32 | A | N | |
| ATOM | 2146 | CA | GLU | A | 896 | 28.657 | 34.727 | −18.183 | 1.00 | 37.36 | A | C | |
| ATOM | 2147 | CB | GLU | A | 896 | 29.923 | 34.720 | −19.038 | 1.00 | 41.29 | A | C | |
| ATOM | 2148 | CG | GLU | A | 896 | 30.283 | 33.289 | −19.467 | 1.00 | 46.94 | A | C | |
| ATOM | 2149 | CD | GLU | A | 896 | 31.666 | 33.172 | −20.076 | 1.00 | 51.83 | A | C | |
| ATOM | 2150 | OE1 | GLU | A | 896 | 32.660 | 33.317 | −19.315 | 1.00 | 54.87 | A | O | |
| ATOM | 2151 | OE2 | GLU | A | 896 | 31.760 | 32.938 | −21.313 | 1.00 | 52.53 | A | O | |
| ATOM | 2152 | C | GLU | A | 896 | 28.953 | 34.051 | −16.863 | 1.00 | 34.85 | A | C | |
| ATOM | 2153 | O | GLU | A | 896 | 29.876 | 34.440 | −16.131 | 1.00 | 33.52 | A | O | |
| ATOM | 2154 | N | PRO | A | 897 | 28.159 | 33.015 | −16.551 | 1.00 | 32.98 | A | N | |
| ATOM | 2155 | CD | PRO | A | 897 | 27.112 | 32.567 | −17.491 | 1.00 | 30.01 | A | C | |
| ATOM | 2156 | CA | PRO | A | 897 | 28.178 | 32.160 | −15.362 | 1.00 | 31.76 | A | C | |
| ATOM | 2157 | CB | PRO | A | 897 | 27.444 | 30.919 | −15.841 | 1.00 | 31.03 | A | C | |
| ATOM | 2158 | CG | PRO | A | 897 | 26.383 | 31.504 | −16.904 | 1.00 | 30.70 | A | C | |
| ATOM | 2159 | C | PRO | A | 897 | 29.542 | 31.841 | −14.757 | 1.00 | 32.40 | A | C | |
| ATOM | 2160 | O | PRO | A | 897 | 29.647 | 31.622 | −13.541 | 1.00 | 31.30 | A | O | |
| ATOM | 2161 | N | THR | A | 898 | 30.582 | 31.810 | −15.591 | 1.00 | 34.36 | A | N | |
| ATOM | 2162 | CA | THR | A | 898 | 31.939 | 31.506 | −15.110 | 1.00 | 35.46 | A | C | |
| ATOM | 2163 | CB | THR | A | 898 | 32.885 | 31.170 | −16.257 | 1.00 | 35.24 | A | C | |
| ATOM | 2164 | OG1 | THR | A | 898 | 32.263 | 31.514 | −17.510 | 1.00 | 36.79 | A | O | |
| ATOM | 2165 | CG2 | THR | A | 898 | 33.240 | 29.692 | −16.215 | 1.00 | 35.03 | A | C | |
| ATOM | 2166 | C | THR | A | 898 | 32.581 | 32.631 | −14.317 | 1.00 | 36.08 | A | C | |
| ATOM | 2167 | O | THR | A | 898 | 33.424 | 32.381 | −13.448 | 1.00 | 37.44 | A | O | |
| ATOM | 2168 | N | HIS | A | 899 | 32.191 | 33.871 | −14.600 | 1.00 | 36.64 | A | N | |
| ATOM | 2169 | CA | HIS | A | 899 | 32.766 | 34.985 | −13.867 | 1.00 | 36.85 | A | C | |
| ATOM | 2170 | CB | HIS | A | 899 | 32.853 | 36.221 | −14.758 | 1.00 | 40.74 | A | C | |
| ATOM | 2171 | CG | HIS | A | 899 | 34.144 | 36.301 | −15.513 | 1.00 | 46.62 | A | C | |
| ATOM | 2172 | CD2 | HIS | A | 899 | 34.550 | 35.681 | −16.649 | 1.00 | 48.81 | A | C | |
| ATOM | 2173 | ND1 | HIS | A | 899 | 35.241 | 36.995 | −15.042 | 1.00 | 49.33 | A | N | |
| ATOM | 2174 | CE1 | HIS | A | 899 | 36.267 | 36.796 | −15.853 | 1.00 | 50.49 | A | C | |
| ATOM | 2175 | NE2 | HIS | A | 899 | 35.875 | 36.001 | −16.835 | 1.00 | 50.88 | A | N | |
| ATOM | 2176 | C | HIS | A | 899 | 32.074 | 35.225 | −12.550 | 1.00 | 34.73 | A | C | |
| ATOM | 2177 | O | HIS | A | 899 | 32.647 | 35.995 | −11.714 | 1.00 | 34.91 | A | O | |
| ATOM | 2178 | N | ARG | A | 900 | 30.854 | 34.791 | −12.349 | 1.00 | 32.24 | A | N | |
| ATOM | 2179 | CA | ARG | A | 900 | 30.161 | 35.021 | −11.079 | 1.00 | 29.47 | A | C | |
| ATOM | 2180 | CB | ARG | A | 900 | 28.801 | 34.338 | −11.030 | 1.00 | 28.72 | A | C | |
| ATOM | 2181 | CG | ARG | A | 900 | 27.785 | 34.844 | −11.981 | 1.00 | 25.86 | A | C | |
| ATOM | 2182 | CD | ARG | A | 900 | 26.457 | 34.238 | −11.657 | 1.00 | 24.92 | A | C | |
| ATOM | 2183 | NE | ARG | A | 900 | 25.509 | 34.630 | −12.680 | 1.00 | 26.84 | A | N | |
| ATOM | 2184 | CZ | ARG | A | 900 | 25.319 | 33.945 | −13.799 | 1.00 | 27.90 | A | C | |
| ATOM | 2185 | NH1 | ARG | A | 900 | 26.000 | 32.831 | −14.012 | 1.00 | 29.25 | A | N | |
| ATOM | 2186 | NH2 | ARG | A | 900 | 24.483 | 34.392 | −14.718 | 1.00 | 28.21 | A | N | |
| ATOM | 2187 | C | ARG | A | 900 | 31.027 | 34.358 | −10.032 | 1.00 | 27.68 | A | C | |
| ATOM | 2188 | O | ARG | A | 900 | 31.747 | 33.409 | −10.329 | 1.00 | 29.47 | A | O | |
| ATOM | 2189 | N | PRO | A | 901 | 30.954 | 34.820 | −8.785 | 1.00 | 25.62 | A | N | |
| ATOM | 2190 | CD | PRO | A | 901 | 30.173 | 35.941 | −8.223 | 1.00 | 24.12 | A | C | |
| ATOM | 2191 | CA | PRO | A | 901 | 31.795 | 34.177 | −7.769 | 1.00 | 24.91 | A | C | |
| ATOM | 2192 | CB | PRO | A | 901 | 31.874 | 35.244 | −6.682 | 1.00 | 23.22 | A | C | |
| ATOM | 2193 | CG | PRO | A | 901 | 30.475 | 35.839 | −6.721 | 1.00 | 22.97 | A | C | |
| ATOM | 2194 | C | PRO | A | 901 | 31.142 | 32.887 | −7.262 | 1.00 | 25.00 | A | C | |
| ATOM | 2195 | O | PRO | A | 901 | 30.260 | 32.305 | −7.927 | 1.00 | 23.82 | A | O | |
| ATOM | 2196 | N | THR | A | 902 | 31.565 | 32.449 | −6.077 | 1.00 | 24.36 | A | N | |
| ATOM | 2197 | CA | THR | A | 902 | 30.989 | 31.256 | −5.455 | 1.00 | 24.43 | A | C | |
| ATOM | 2198 | CB | THR | A | 902 | 32.024 | 30.159 | −5.300 | 1.00 | 23.14 | A | C | |
| ATOM | 2199 | OG | THR | A | 902 | 33.224 | 30.721 | −4.729 | 1.00 | 23.76 | A | O | |
| ATOM | 2200 | CG2 | THR | A | 902 | 32.301 | 29.515 | −6.649 | 1.00 | 21.07 | A | C | |
| ATOM | 2201 | C | THR | A | 902 | 30.472 | 31.601 | −4.062 | 1.00 | 24.46 | A | C | |
| ATOM | 2202 | O | THR | A | 902 | 30.921 | 32.570 | −3.440 | 1.00 | 23.55 | A | O | |
| ATOM | 2203 | N | PHE | A | 903 | 29.537 | 30.806 | −3.559 | 1.00 | 25.02 | A | N | |
| ATOM | 2204 | CA | PHE | A | 903 | 29.008 | 31.082 | −2.234 | 1.00 | 26.45 | A | C | |
| ATOM | 2205 | CB | PHE | A | 903 | 27.949 | 30.048 | −1.883 | 1.00 | 26.28 | A | C | |
| ATOM | 2206 | CG | PHE | A | 903 | 26.711 | 30.183 | −2.720 | 1.00 | 26.42 | A | C | |
| ATOM | 2207 | CD1 | PHE | A | 903 | 25.952 | 31.351 | −2.664 | 1.00 | 24.63 | A | C | |
| ATOM | 2208 | CD2 | PHE | A | 903 | 26.307 | 29.154 | −3.573 | 1.00 | 25.83 | A | C | |
| ATOM | 2209 | CE1 | PHE | A | 903 | 24.796 | 31.495 | −3.445 | 1.00 | 26.30 | A | C | |
| ATOM | 2210 | CE2 | PHE | A | 903 | 25.158 | 29.284 | −4.358 | 1.00 | 25.43 | A | C | |

REMARK c-fms (538-922, FGF chimera) complexed with 793693
REMARK refinement resolution: 500.0-2.8 A
REMARK starting r = 0.2645 free_r = 0.3191
REMARK final = 0.2617 free_r = 0.3141
CRYST1 82.210 82.210 143.440 90.00 90.00 120.00 R 3
REMARK Written by CNX VERSION: 2000.12

TABLE 3
Coordinates of c-FMS (tie-2-chimera) in complex with 1183648 (Arylamide)

| | | | | | | | | | | | | | |
|---|---|---|---|---|---|---|---|---|---|---|---|---|---|
| ATOM | 1 | CB | GLN | A | 547 | 50.705 | 48.088 | 11.065 | 1.00 | 59.29 | A | C | |
| ATOM | 2 | CG | GLN | A | 547 | 50.108 | 48.650 | 9.788 | 1.00 | 63.33 | A | C | |
| ATOM | 3 | CD | GLN | A | 547 | 48.603 | 48.477 | 9.735 | 1.00 | 65.77 | A | C | |
| ATOM | 4 | OE1 | GLN | A | 547 | 47.870 | 49.057 | 10.538 | 1.00 | 67.17 | A | O | |
| ATOM | 5 | NE2 | GLN | A | 547 | 48.134 | 47.667 | 8.790 | 1.00 | 64.89 | A | N | |
| ATOM | 6 | C | GLN | A | 547 | 50.754 | 45.684 | 10.330 | 1.00 | 57.19 | A | C | |
| ATOM | 7 | O | GLN | A | 547 | 51.326 | 44.642 | 10.662 | 1.00 | 59.65 | A | O | |
| ATOM | 8 | N | GLN | A | 547 | 50.739 | 46.267 | 12.745 | 1.00 | 59.16 | A | N | |
| ATOM | 9 | CA | GLN | A | 547 | 50.248 | 46.662 | 11.392 | 1.00 | 57.65 | A | C | |
| ATOM | 10 | N | VAL | A | 548 | 50.548 | 46.015 | 9.058 | 1.00 | 49.89 | A | N | |
| ATOM | 11 | CA | VAL | A | 548 | 50.976 | 45.139 | 7.979 | 1.00 | 47.64 | A | C | |
| ATOM | 12 | CB | VAL | A | 548 | 49.919 | 44.026 | 7.743 | 1.00 | 51.23 | A | C | |
| ATOM | 13 | CG1 | VAL | A | 548 | 48.733 | 44.574 | 6.957 | 1.00 | 52.01 | A | C | |
| ATOM | 14 | CG2 | VAL | A | 548 | 50.551 | 42.851 | 7.046 | 1.00 | 52.53 | A | C | |
| ATOM | 15 | C | VAL | A | 548 | 51.222 | 45.909 | 6.678 | 1.00 | 46.21 | A | C | |
| ATOM | 16 | O | VAL | A | 548 | 51.047 | 45.373 | 5.582 | 1.00 | 44.14 | A | O | |
| ATOM | 17 | N | ARG | A | 549 | 51.650 | 47.162 | 6.806 | 1.00 | 37.38 | A | N | |
| ATOM | 18 | CA | ARG | A | 549 | 51.924 | 48.005 | 5.647 | 1.00 | 30.25 | A | C | |
| ATOM | 19 | CB | ARG | A | 549 | 51.137 | 49.311 | 5.763 | 1.00 | 32.08 | A | C | |
| ATOM | 20 | CG | ARG | A | 549 | 49.672 | 49.113 | 6.157 | 1.00 | 43.17 | A | C | |
| ATOM | 21 | CD | ARG | A | 549 | 48.938 | 50.441 | 6.288 | 1.00 | 55.28 | A | C | |
| ATOM | 22 | NE | ARG | A | 549 | 47.580 | 50.289 | 6.814 | 1.00 | 61.34 | A | N | |
| ATOM | 23 | CZ | ARG | A | 549 | 46.614 | 49.591 | 6.221 | 1.00 | 64.15 | A | C | |
| ATOM | 24 | NH1 | ARG | A | 549 | 46.844 | 48.969 | 5.072 | 1.00 | 66.91 | A | N | |
| ATOM | 25 | NH2 | ARG | A | 549 | 45.413 | 49.517 | 6.779 | 1.00 | 64.80 | A | N | |
| ATOM | 26 | C | ARG | A | 549 | 53.420 | 48.320 | 5.535 | 1.00 | 27.01 | A | C | |
| ATOM | 27 | O | ARG | A | 549 | 54.156 | 48.272 | 6.525 | 1.00 | 22.12 | A | O | |
| ATOM | 28 | N | TRP | A | 550 | 53.872 | 48.645 | 4.327 | 1.00 | 19.84 | A | N | |
| ATOM | 29 | CA | TRP | A | 550 | 55.276 | 48.980 | 4.125 | 1.00 | 21.93 | A | C | |
| ATOM | 30 | CB | TRP | A | 550 | 55.556 | 49.244 | 2.650 | 1.00 | 19.89 | A | C | |
| ATOM | 31 | CG | TRP | A | 550 | 55.585 | 48.012 | 1.850 | 1.00 | 24.51 | A | C | |
| ATOM | 32 | CD2 | TRP | A | 550 | 56.193 | 47.837 | 0.567 | 1.00 | 22.47 | A | C | |
| ATOM | 33 | CE2 | TRP | A | 550 | 55.994 | 46.492 | 0.193 | 1.00 | 20.46 | A | C | |
| ATOM | 34 | CE3 | TRP | A | 550 | 56.889 | 48.685 | −0.298 | 1.00 | 18.32 | A | C | |
| ATOM | 35 | CD1 | TRP | A | 550 | 55.051 | 46.801 | 2.194 | 1.00 | 16.54 | A | C | |
| ATOM | 36 | NE1 | TRP | A | 550 | 55.293 | 45.882 | 1.201 | 1.00 | 20.30 | A | N | |
| ATOM | 37 | CZ2 | TRP | A | 550 | 56.465 | 45.976 | −1.010 | 1.00 | 18.45 | A | C | |
| ATOM | 38 | CZ3 | TRP | A | 550 | 57.355 | 48.176 | −1.489 | 1.00 | 20.72 | A | C | |
| ATOM | 39 | CH2 | TRP | A | 550 | 57.142 | 46.830 | −1.838 | 1.00 | 25.45 | A | C | |
| ATOM | 40 | C | TRP | A | 550 | 55.583 | 50.229 | 4.935 | 1.00 | 24.49 | A | C | |
| ATOM | 41 | O | TRP | A | 550 | 54.742 | 51.125 | 5.045 | 1.00 | 20.67 | A | O | |
| ATOM | 42 | N | LYS | A | 551 | 56.790 | 50.292 | 5.481 | 1.00 | 19.05 | A | N | |
| ATOM | 43 | CA | LYS | A | 551 | 57.191 | 51.433 | 6.302 | 1.00 | 23.41 | A | C | |
| ATOM | 44 | CB | LYS | A | 551 | 56.747 | 51.202 | 7.747 | 1.00 | 26.59 | A | C | |
| ATOM | 45 | CG | LYS | A | 551 | 57.092 | 52.332 | 8.709 | 1.00 | 32.32 | A | C | |
| ATOM | 46 | CD | LYS | A | 551 | 56.462 | 52.081 | 10.073 | 1.00 | 45.89 | A | C | |
| ATOM | 47 | CE | LYS | A | 551 | 56.653 | 53.267 | 11.008 | 1.00 | 48.10 | A | C | |
| ATOM | 48 | NZ | LYS | A | 551 | 58.091 | 53.564 | 11.235 | 1.00 | 51.66 | A | N | |
| ATOM | 49 | C | LYS | A | 551 | 58.692 | 51.665 | 6.285 | 1.00 | 23.21 | A | C | |
| ATOM | 50 | O | LYS | A | 551 | 59.468 | 50.722 | 6.438 | 1.00 | 22.75 | A | O | |
| ATOM | 51 | N | ILE | A | 552 | 59.086 | 52.925 | 6.106 | 1.00 | 22.70 | A | N | |
| ATOM | 52 | CA | ILE | A | 552 | 60.490 | 53.315 | 6.116 | 1.00 | 28.63 | A | C | |
| ATOM | 53 | CB | ILE | A | 552 | 60.722 | 54.668 | 5.423 | 1.00 | 30.18 | A | C | |
| ATOM | 54 | CG2 | ILE | A | 552 | 62.187 | 55.072 | 5.575 | 1.00 | 28.32 | A | C | |
| ATOM | 55 | CG1 | ILE | A | 552 | 60.295 | 54.594 | 3.952 | 1.00 | 28.75 | A | C | |
| ATOM | 56 | CD1 | ILE | A | 552 | 61.101 | 53.642 | 3.116 | 1.00 | 26.35 | A | C | |
| ATOM | 57 | C | ILE | A | 552 | 60.851 | 53.490 | 7.591 | 1.00 | 29.56 | A | C | |
| ATOM | 58 | O | ILE | A | 552 | 60.209 | 54.260 | 8.298 | 1.00 | 31.45 | A | O | |
| ATOM | 59 | N | ILE | A | 553 | 61.870 | 52.773 | 8.048 | 1.00 | 24.52 | A | N | |
| ATOM | 60 | CA | ILE | A | 553 | 62.294 | 52.851 | 9.437 | 1.00 | 27.47 | A | C | |
| ATOM | 61 | CB | ILE | A | 553 | 62.485 | 51.447 | 10.040 | 1.00 | 34.69 | A | C | |
| ATOM | 62 | CG2 | ILE | A | 553 | 61.199 | 50.641 | 9.912 | 1.00 | 37.60 | A | C | |
| ATOM | 63 | CG1 | ILE | A | 5.53 | 63.642 | 50.736 | 9.335 | 1.00 | 32.96 | A | C | |
| ATOM | 64 | CD1 | ILE | A | 553 | 63.957 | 49.375 | 9.911 | 1.00 | 42.39 | A | C | |
| ATOM | 65 | C | ILE | A | 553 | 63.614 | 53.602 | 9.561 | 1.00 | 29.91 | A | C | |
| ATOM | 66 | O | ILE | A | 553 | 64.309 | 53.824 | 8.569 | 1.00 | 25.11 | A | O | |
| ATOM | 67 | N | GLU | A | 554 | 63.956 | 53.981 | 10.790 | 1.00 | 37.63 | A | N | |
| ATOM | 68 | CA | GLU | A | 554 | 65.196 | 54.709 | 11.059 | 1.00 | 42.70 | A | C | |
| ATOM | 69 | CB | GLU | A | 554 | 65.035 | 55.577 | 12.311 | 1.00 | 44.36 | A | C | |
| ATOM | 70 | CG | GLU | A | 554 | 64.018 | 56.693 | 12.177 | 1.00 | 55.55 | A | C | |
| ATOM | 71 | CD | GLU | A | 554 | 63.794 | 57.434 | 13.483 | 1.00 | 62.27 | A | C | |
| ATOM | 72 | OE1 | GLU | A | 554 | 63.035 | 58.428 | 13.479 | 1.00 | 65.84 | A | O | |
| ATOM | 73 | OE2 | GLU | A | 554 | 64.371 | 57.022 | 14.513 | 1.00 | 65.19 | A | O | |
| ATOM | 74 | C | GLU | A | 554 | 66.401 | 53.789 | 11.248 | 1.00 | 42.46 | A | C | |
| ATOM | 75 | O | GLU | A | 554 | 66.362 | 52.853 | 12.042 | 1.00 | 44.89 | A | O | |
| ATOM | 76 | N | SER | A | 555 | 67.472 | 54.070 | 10.512 | 1.00 | 45.78 | A | N | |
| ATOM | 77 | CA | SER | A | 555 | 68.706 | 53.298 | 10.600 | 1.00 | 49.70 | A | C | |
| ATOM | 78 | CB | SER | A | 555 | 68.556 | 51.932 | 9.921 | 1.00 | 57.95 | A | C | |
| ATOM | 79 | OG | SER | A | 555 | 67.937 | 50.989 | 10.783 | 1.00 | 57.72 | A | O | |
| ATOM | 80 | C | SER | A | 555 | 69.834 | 54.076 | 9.945 | 1.00 | 56.37 | A | C | |
| ATOM | 81 | O | SER | A | 555 | 69.588 | 55.025 | 9.195 | 1.00 | 55.73 | A | O | |
| ATOM | 82 | N | TYR | A | 556 | 71.070 | 53.673 | 10.231 | 1.00 | 61.77 | A | N | |
| ATOM | 83 | CA | TYR | A | 556 | 72.243 | 54.344 | 9.681 | 1.00 | 63.94 | A | C | |
| ATOM | 84 | CB | TYR | A | 556 | 72.460 | 55.675 | 10.404 | 1.00 | 68.76 | A | C | |
| ATOM | 85 | CG | TYR | A | 556 | 72.226 | 55.605 | 11.894 | 1.00 | 71.33 | A | C | |
| ATOM | 86 | CD1 | TYR | A | 556 | 72.998 | 54.779 | 12.702 | 1.00 | 76.60 | A | C | |
| ATOM | 87 | CE1 | TYR | A | 556 | 72.770 | 54.695 | 14.065 | 1.00 | 76.88 | A | C | |
| ATOM | 88 | CD2 | TYR | A | 556 | 71.217 | 56.349 | 12.490 | 1.00 | 73.84 | A | C | |
| ATOM | 89 | CE2 | TYR | A | 556 | 70.981 | 56.272 | 13.850 | 1.00 | 77.97 | A | C | |
| ATOM | 90 | CZ | TYR | A | 556 | 71.759 | 55.443 | 14.634 | 1.00 | 77.93 | A | C | |
| ATOM | 91 | OH | TYR | A | 556 | 71.519 | 55.359 | 15.988 | 1.00 | 77.63 | A | O | |
| ATOM | 92 | C | TYR | A | 556 | 73.507 | 53.495 | 9.777 | 1.00 | 65.66 | A | C | |
| ATOM | 93 | O | TYR | A | 556 | 74.618 | 53.999 | 9.592 | 1.00 | 63.62 | A | O | |
| ATOM | 94 | N | ASN | A | 559 | 74.913 | 50.418 | 6.858 | 1.00 | 58.03 | A | N | |
| ATOM | 95 | CA | ASN | A | 559 | 74.977 | 50.113 | 5.433 | 1.00 | 55.60 | A | C | |
| ATOM | 96 | CB | ASN | A | 559 | 74.564 | 48.659 | 5.195 | 1.00 | 58.24 | A | C | |
| ATOM | 97 | CG | ASN | A | 559 | 74.936 | 48.165 | 3.813 | 1.00 | 64.11 | A | C | |
| ATOM | 98 | OD1 | ASN | A | 559 | 74.469 | 48.692 | 2.801 | 1.00 | 66.81 | A | O | |
| ATOM | 99 | ND2 | ASN | A | 559 | 75.786 | 47.141 | 3.762 | 1.00 | 63.62 | A | N | |
| ATOM | 100 | C | ASN | A | 559 | 74.024 | 51.059 | 4.710 | 1.00 | 53.13 | A | C | |
| ATOM | 101 | O | ASN | A | 559 | 74.407 | 51.752 | 3.765 | 1.00 | 50.85 | A | O | |
| ATOM | 102 | N | SER | A | 560 | 72.776 | 51.076 | 5.167 | 1.00 | 49.61 | A | N | |
| ATOM | 103 | CA | SER | A | 560 | 71.758 | 51.951 | 4.604 | 1.00 | 46.25 | A | C | |
| ATOM | 104 | CB | SER | A | 560 | 70.430 | 51.204 | 4.482 | 1.00 | 49.55 | A | C | |
| ATOM | 105 | OG | SER | A | 560 | 69.445 | 52.027 | 3.883 | 1.00 | 61.42 | A | O | |
| ATOM | 106 | C | SER | A | 560 | 71.608 | 53.135 | 5.558 | 1.00 | 38.67 | A | C | |
| ATOM | 107 | O | SER | A | 560 | 71.898 | 53.016 | 6.749 | 1.00 | 36.71 | A | O | |
| ATOM | 108 | N | TYR | A | 561 | 71.161 | 54.275 | 5.045 | 1.00 | 35.17 | A | N | |
| ATOM | 109 | CA | TYR | A | 561 | 71.006 | 55.458 | 5.888 | 1.00 | 34.70 | A | C | |
| ATOM | 110 | CE | TYR | A | 561 | 72.177 | 56.430 | 5.647 | 1.00 | 28.11 | A | C | |
| ATOM | 111 | CG | TYR | A | 561 | 72.291 | 57.536 | 6.675 | 1.00 | 30.02 | A | C | |
| ATOM | 112 | CD1 | TYR | A | 561 | 71.482 | 58.663 | 6.612 | 1.00 | 26.28 | A | C | |
| ATOM | 113 | CE1 | TYR | A | 561 | 71.554 | 59.648 | 7.577 | 1.00 | 31.92 | A | C | |
| ATOM | 114 | CD2 | TYR | A | 561 | 73.182 | 57.429 | 7.736 | 1.00 | 30.58 | A | C | |
| ATOM | 115 | CE2 | TYR | A | 561 | 73.260 | 58.405 | 8.706 | 1.00 | 23.12 | A | C | |
| ATOM | 116 | CZ | TYR | A | 561 | 72.443 | 59.513 | 8.620 | 1.00 | 28.95 | A | C | |
| ATOM | 117 | OH | TYR | A | 561 | 72.525 | 60.490 | 9.584 | 1.00 | 31.30 | A | O | |
| ATOM | 118 | C | TYR | A | 561 | 69.671 | 56.150 | 5.628 | 1.00 | 31.68 | A | C | |
| ATOM | 119 | O | TYR | A | 561 | 69.323 | 56.447 | 4.489 | 1.00 | 29.98 | A | O | |
| ATOM | 120 | N | THR | A | 562 | 68.916 | 56.391 | 6.692 | 1.00 | 32.64 | A | N | |
| ATOM | 121 | CA | THR | A | 562 | 67.624 | 57.056 | 6.553 | 1.00 | 28.44 | A | C | |
| ATOM | 122 | CB | THR | A | 562 | 66.618 | 56.567 | 7.623 | 1.00 | 31.76 | A | C | |
| ATOM | 123 | OG1 | THR | A | 562 | 66.542 | 55.135 | 7.597 | 1.00 | 42.23 | A | 0 | |
| ATOM | 124 | CG2 | THR | A | 562 | 65.230 | 57.138 | 7.347 | 1.00 | 35.49 | A | C | |
| ATOM | 125 | C | THR | A | 562 | 67.821 | 58.562 | 6.717 | 1.00 | 26.81 | A | C | |
| ATOM | 126 | O | THR | A | 562 | 67.944 | 59.059 | 7.837 | 1.00 | 25.47 | A | 0 | |
| ATOM | 127 | N | PHE | A | 563 | 67.876 | 59.276 | 5.598 | 1.00 | 18.63 | A | N | |
| ATOM | 128 | CA | PHE | A | 563 | 68.033 | 60.724 | 5.606 | 1.00 | 24.24 | A | C | |
| ATOM | 129 | CB | PHE | A | 563 | 68.578 | 61.231 | 4.263 | 1.00 | 28.51 | A | C | |
| ATOM | 130 | CG | PHE | A | 563 | 70.026 | 60.897 | 4.017 | 1.00 | 26.30 | A | C | |
| ATOM | 131 | CD1 | PHE | A | 563 | 70.389 | 59.706 | 3.401 | 1.00 | 29.06 | A | C | |
| ATOM | 132 | CD2 | PHE | A | 563 | 71.024 | 61.789 | 4.384 | 1.00 | 29.63 | A | C | |
| ATOM | 133 | CE1 | PHE | A | 563 | 71.722 | 59.413 | 3.152 | 1.00 | 34.72 | A | C | |
| ATOM | 134 | CE2 | PHE | A | 563 | 72.360 | 61.503 | 4.140 | 1.00 | 28.25 | A | C | |
| ATOM | 135 | CZ | PHE | A | 563 | 72.711 | 60.315 | 3.523 | 1.00 | 28.14 | A | C | |
| ATOM | 136 | C | PHE | A | 563 | 66.677 | 61.379 | 5.853 | 1.00 | 31.10 | A | C | |
| ATOM | 137 | O | PHE | A | 563 | 66.599 | 62.493 | 6.378 | 1.00 | 24.85 | A | 0 | |
| ATOM | 138 | N | ILE | A | 564 | 65.608 | 60.692 | 5.460 | 1.00 | 29.30 | A | N | |
| ATOM | 139 | CA | ILE | A | 564 | 64.269 | 61.232 | 5.651 | 1.00 | 28.37 | A | C | |
| ATOM | 140 | CB | ILE | A | 564 | 63.990 | 62.373 | 4.644 | 1.00 | 34.24 | A | C | |
| ATOM | 141 | CG2 | ILE | A | 564 | 63.888 | 61.812 | 3.227 | 1.00 | 29.15 | A | C | |
| ATOM | 142 | CG1 | ILE | A | 564 | 62.690 | 63.086 | 5.015 | 1.00 | 38.26 | A | C | |
| ATOM | 143 | CD1 | ILE | A | 564 | 62.388 | 64.284 | 4.141 | 1.00 | 46.88 | A | C | |
| ATOM | 144 | C | ILE | A | 564 | 63.164 | 60.189 | 5.512 | 1.00 | 30.93 | A | C | |
| ATOM | 145 | O | ILE | A | 564 | 63.285 | 59.234 | 4.745 | 1.00 | 34.04 | A | O | |
| ATOM | 146 | N | ASP | A | 565 | 62.097 | 60.378 | 6.280 | 1.00 | 32.11 | A | N | |
| ATOM | 147 | CA | ASP | A | 565 | 60.932 | 59.508 | 6.234 | 1.00 | 27.33 | A | C | |
| ATOM | 148 | CB | ASP | A | 565 | 60.328 | 59.347 | 7.633 | 1.00 | 28.72 | A | C | |
| ATOM | 149 | CG | ASP | A | 565 | 59.029 | 58.551 | 7.623 | 1.00 | 38.03 | A | C | |
| ATOM | 150 | OD1 | ASP | A | 565 | 58.503 | 58.271 | 8.721 | 1.00 | 37.84 | A | O | |
| ATOM | 151 | OD2 | ASP | A | 565 | 58.533 | 58.208 | 6.527 | 1.00 | 26.21 | A | O | |
| ATOM | 152 | C | ASP | A | 565 | 59.933 | 60.197 | 5.312 | 1.00 | 30.22 | A | C | |
| ATOM | 153 | O | ASP | A | 565 | 59.357 | 61.229 | 5.663 | 1.00 | 33.03 | A | O | |
| ATOM | 154 | N | PRO | A | 566 | 59.734 | 59.643 | 4.108 | 1.00 | 33.59 | A | N | |
| ATOM | 155 | CD | PRO | A | 566 | 60.354 | 58.379 | 3.686 | 1.00 | 34.17 | A | C | |
| ATOM | 156 | CA | PRO | A | 566 | 58.821 | 60.141 | 3.073 | 1.00 | 34.98 | A | C | |
| ATOM | 157 | CB | PRO | A | 566 | 58.851 | 59.034 | 2.023 | 1.00 | 35.51 | A | C | |
| ATOM | 158 | CG | PRO | A | 566 | 60.177 | 58.434 | 2.198 | 1.00 | 37.05 | A | C | |
| ATOM | 159 | C | PRO | A | 566 | 57.408 | 60.344 | 3.614 | 1.00 | 33.80 | A | C | |
| ATOM | 160 | O | PRO | A | 566 | 56.650 | 61.169 | 3.099 | 1.00 | 32.70 | A | O | |
| ATOM | 161 | N | THR | A | 567 | 57.066 | 59.571 | 4.645 | 1.00 | 32.01 | A | N | |
| ATOM | 162 | CA | THR | A | 567 | 55.746 | 59.630 | 5.271 | 1.00 | 36.01 | A | C | |
| ATOM | 163 | CB | THR | A | 567 | 55.638 | 58.638 | 6.442 | 1.00 | 34.80 | A | C | |
| ATOM | 164 | OG1 | THR | A | 567 | 55.699 | 57.299 | 5.932 | 1.00 | 43.97 | A | O | |
| ATOM | 165 | CG2 | THR | A | 567 | 54.329 | 58.832 | 7.191 | 1.00 | 46.71 | A | C | |
| ATOM | 166 | C | THR | A | 567 | 55.448 | 61.031 | 5.766 | 1.00 | 41.90 | A | C | |
| ATOM | 167 | O | THR | A | 567 | 54.376 | 61.571 | 5.493 | 1.00 | 46.23 | A | O | |
| ATOM | 168 | N | GLN | A | 568 | 56.382 | 61.618 | 6.510 | 1.00 | 43.71 | A | N | |
| ATOM | 169 | CA | GLN | A | 568 | 56.190 | 62.984 | 6.964 | 1.00 | 48.14 | A | C | |
| ATOM | 170 | CB | GLN | A | 568 | 57.317 | 63.420 | 7.902 | 1.00 | 44.17 | A | C | |
| ATOM | 171 | CG | GLN | A | 568 | 57.140 | 63.012 | 9.354 | 1.00 | 50.98 | A | C | |
| ATOM | 172 | CD | GLN | A | 568 | 57.166 | 61.513 | 9.562 | 1.00 | 57.27 | A | C | |
| ATOM | 173 | OE1 | GLN | A | 568 | 58.117 | 60.834 | 9.174 | 1.00 | 58.66 | A | O | |
| ATOM | 174 | NE2 | GLN | A | 568 | 56.119 | 60.987 | 10.188 | 1.00 | 59.35 | A | N | |
| ATOM | 175 | C | GLN | A | 568 | 56.256 | 63.780 | 5.666 | 1.00 | 51.91 | A | C | |
| ATOM | 176 | O | GLN | A | 568 | 56.617 | 63.231 | 4.623 | 1.00 | 55.37 | A | O | |
| ATOM | 177 | N | LEU | A | 569 | 55.917 | 65.060 | 5.719 | 1.00 | 55.50 | A | N | |
| ATOM | 178 | CA | LEU | A | 569 | 55.948 | 65.895 | 4.522 | 1.00 | 56.69 | A | C | |
| ATOM | 179 | CB | LEU | A | 569 | 57.295 | 65.762 | 3.794 | 1.00 | 59.47 | A | C | |
| ATOM | 180 | CG | LEU | A | 569 | 58.623 | 66.112 | 4.474 | 1.00 | 64.81 | A | C | |
| ATOM | 181 | CD1 | LEU | A | 569 | 59.00 | 65.058 | 5.507 | 1.00 | 65.18 | A | C | |
| ATOM | 182 | CD2 | LEU | A | 569 | 59.708 | 66.192 | 3.406 | 1.00 | 68.71 | A | C | |
| ATOM | 183 | C | LEU | A | 569 | 54.838 | 65.505 | 3.549 | 1.00 | 55.23 | A | C | |
| ATOM | 184 | O | LEU | A | 569 | 54.490 | 64.327 | 3.416 | 1.00 | 50.24 | A | O | |
| ATOM | 185 | N | PRO | A | 570 | 54.259 | 66.497 | 2.861 | 1.00 | 53.48 | A | N | |
| ATOM | 186 | CD | PRO | A | 570 | 54.362 | 67.938 | 3.155 | 1.00 | 49.89 | A | C | |
| ATOM | 187 | CA | PRO | A | 570 | 53.190 | 66.233 | 1.894 | 1.00 | 53.48 | A | C | |
| ATOM | 188 | CB | PRO | A | 570 | 52.818 | 67.632 | 1.413 | 1.00 | 51.63 | A | C | |
| ATOM | 189 | CG | PRO | A | 570 | 53.035 | 68.461 | 2.652 | 1.00 | 53.16 | A | C | |
| ATOM | 190 | C | PRO | A | 570 | 53.682 | 65.327 | 0.760 | 1.00 | 54.61 | A | C | |
| ATOM | 191 | O | PRO | A | 570 | 54.751 | 64.726 | 0.857 | 1.00 | 55.35 | A | O | |
| ATOM | 192 | N | TYR | A | 571 | 52.903 | 65.246 | −0.314 | 1.00 | 56.06 | A | N | |
| ATOM | 193 | CA | TYR | A | 571 | 53.226 | 64.405 | −1.467 | 1.00 | 54.29 | A | C | |
| ATOM | 194 | CB | TYR | A | 571 | 51.949 | 63.676 | −1.910 | 1.00 | 45.30 | A | C | |
| ATOM | 195 | CG | TYR | A | 571 | 51.813 | 63.445 | −3.397 | 1.00 | 45.06 | A | C | |
| ATOM | 196 | CD1 | TYR | A | 571 | 52.552 | 62.461 | −4.049 | 1.00 | 40.56 | A | C | |
| ATOM | 197 | CE1 | TYR | A | 571 | 52.414 | 62.250 | −5.411 | 1.00 | 36.51 | A | C | |
| ATOM | 198 | CD2 | TYR | A | 571 | 50.935 | 64.212 | −4.151 | 1.00 | 37.84 | A | C | |
| ATOM | 199 | CE2 | TYR | A | 571 | 50.792 | 64.011 | −5.507 | 1.00 | 39.91 | A | C | |
| ATOM | 200 | CZ | TYR | A | 571 | 51.531 | 63.031 | −6.133 | 1.00 | 36.18 | A | C | |
| ATOM | 201 | OH | TYR | A | 571 | 51.382 | 62.843 | −7.488 | 1.00 | 44.41 | A | O | |
| ATOM | 202 | C | TYR | A | 571 | 53.849 | 65.182 | −2.636 | 1.00 | 57.33 | A | C | |
| ATOM | 203 | O | TYR | A | 571 | 53.670 | 66.392 | −2.753 | 1.00 | 59.52 | A | O | |
| ATOM | 204 | N | ASN | A | 572 | 54.579 | 64.468 | −3.493 | 1.00 | 60.54 | A | N | |
| ATOM | 205 | CA | ASN | A | 572 | 55.257 | 65.046 | −4.660 | 1.00 | 62.84 | A | C | |
| ATOM | 206 | CB | ASN | A | 572 | 56.530 | 64.253 | −4.961 | 1.00 | 63.18 | A | C | |
| ATOM | 207 | CG | ASN | A | 572 | 57.515 | 64.277 | −3.818 | 1.00 | 66.91 | A | C | |
| ATOM | 208 | OD1 | ASN | A | 572 | 57.133 | 64.151 | −2.656 | 1.00 | 72.16 | A | O | |
| ATOM | 209 | ND2 | ASN | A | 572 | 58.796 | 64.425 | −4.141 | 1.00 | 68.55 | A | N | |
| ATOM | 210 | C | ASN | A | 572 | 54.410 | 65.071 | −5.933 | 1.00 | 62.14 | A | C | |
| ATOM | 211 | O | ASN | A | 572 | 54.284 | 64.051 | −6.609 | 1.00 | 65.57 | A | O | |
| ATOM | 212 | N | GLU | A | 573 | 53.862 | 66.236 | −6.275 | 1.00 | 59.46 | A | N | |
| ATOM | 213 | CA | GLU | A | 573 | 53.039 | 66.394 | −7.480 | 1.00 | 53.77 | A | C | |
| ATOM | 214 | CB | GLU | A | 573 | 52.718 | 67.876 | −7.703 | 1.00 | 53.46 | A | C | |
| ATOM | 215 | CG | GLU | A | 573 | 51.653 | 68.444 | −6.777 | 1.00 | 57.06 | A | C | |
| ATOM | 216 | CD | GLU | A | 573 | 50.261 | 67.918 | −7.095 | 1.00 | 55.91 | A | C | |
| ATOM | 217 | OE1 | GLU | A | 573 | 49.810 | 68.074 | −8.251 | 1.00 | 54.97 | A | O | |
| ATOM | 218 | OE2 | GLU | A | 573 | 49.615 | 67.353 | −6.192 | 1.00 | 50.79 | A | O | |
| ATOM | 219 | C | GLU | A | 573 | 53.722 | 65.837 | −8.729 | 1.00 | 52.29 | A | C | |
| ATOM | 220 | O | GLU | A | 573 | 53.197 | 65.928 | −9.845 | 1.00 | 48.82 | A | O | |
| ATOM | 221 | N | LYS | A | 574 | 54.893 | 65.249 | −8.527 | 1.00 | 51.27 | A | N | |
| ATOM | 222 | CA | LYS | A | 574 | 55.685 | 64.682 | −9.606 | 1.00 | 49.90 | A | C | |
| ATOM | 223 | CB | LYS | A | 574 | 57.096 | 64.392 | −9.090 | 1.00 | 52.80 | A | C | |
| ATOM | 224 | CG | LYS | A | 574 | 57.949 | 65.633 | −8.861 | 1.00 | 57.31 | A | C | |
| ATOM | 225 | CD | LYS | A | 574 | 57.362 | 66.593 | −7.845 | 1.00 | 55.83 | A | C | |
| ATOM | 226 | CE | LYS | A | 574 | 58.257 | 67.821 | −7.692 | 1.00 | 57.27 | A | C | |
| ATOM | 227 | NZ | LYS | A | 574 | 57.701 | 68.832 | −6.750 | 1.00 | 56.52 | A | N | |
| ATOM | 228 | C | LYS | A | 574 | 55.115 | 63.421 | −10.259 | 1.00 | 47.63 | A | C | |
| ATOM | 229 | O | LYS | A | 574 | 55.643 | 62.962 | −11.271 | 1.00 | 45.25 | A | O | |
| ATOM | 230 | N | TRP | A | 575 | 54.043 | 62.863 | −9.701 | 1.00 | 39.10 | A | N | |
| ATOM | 231 | CA | TRP | A | 575 | 53.467 | 61.645 | −10.268 | 1.00 | 34.70 | A | C | |
| ATOM | 232 | CE | TRP | A | 575 | 53.475 | 60.524 | −9.224 | 1.00 | 32.40 | A | C | |
| ATOM | 233 | CG | TRP | A | 575 | 54.836 | 60.076 | −8.867 | 1.00 | 24.25 | A | C | |
| ATOM | 234 | CD2 | TRP | A | 575 | 55.569 | 59.018 | −9.485 | 1.00 | 25.63 | A | C | |
| ATOM | 235 | CE2 | TRP | A | 575 | 56.844 | 58.987 | −8.885 | 1.00 | 29.80 | A | C | |
| ATOM | 236 | CE3 | TRP | A | 575 | 55.272 | 58.093 | −10.492 | 1.00 | 31.67 | A | C | |
| ATOM | 237 | CD1 | TRP | A | 575 | 55.668 | 60.630 | −7.940 | 1.00 | 32.41 | A | C | |
| ATOM | 238 | NE1 | TRP | A | 575 | 56.880 | 59.981 | −7.944 | 1.00 | 28.89 | A | N | |
| ATOM | 239 | CZ2 | TRP | A | 575 | 57.822 | 58.066 | −9.260 | 1.00 | 29.83 | A | C | |
| ATOM | 240 | CZ3 | TRP | A | 575 | 56.244 | 57.183 | −10.861 | 1.00 | 32.60 | A | C | |
| ATOM | 241 | CH2 | TRP | A | 57 | 57.504 | 57.176 | −10.247 | 1.00 | 26.78 | A | C | |
| ATOM | 242 | C | TRP | A | 575 | 52.059 | 61.783 | −10.830 | 1.00 | 35.45 | A | C | |
| ATOM | 243 | O | TRP | A | 575 | 51.495 | 60.820 | −11.355 | 1.00 | 29.00 | A | O | |
| ATOM | 244 | N | GLU | A | 576 | 51.499 | 62.981 | −10.730 | 1.00 | 32.64 | A | N | |
| ATOM | 245 | CA | GLU | A | 576 | 50.147 | 63.232 | −11.206 | 1.00 | 33.90 | A | C | |
| ATOM | 246 | CE | GLU | A | 576 | 49.837 | 64.724 | −11.086 | 1.00 | 34.05 | A | C | |
| ATOM | 247 | CG | GLU | A | 576 | 48.381 | 65.038 | −10.843 | 1.00 | 43.65 | A | C | |
| ATOM | 248 | CD | GLU | A | 576 | 47.811 | 64.254 | −9.680 | 1.00 | 41.94 | A | C | |
| ATOM | 249 | OE1 | GLU | A | 576 | 48.510 | 64.107 | −8.652 | 1.00 | 46.89 | A | O | |
| ATOM | 250 | OE2 | GLU | A | 576 | 46.657 | 63.796 | −9.793 | 1.00 | 46.18 | A | O | |
| ATOM | 251 | C | GLU | A | 576 | 49.933 | 62.756 | −12.642 | 1.00 | 32.44 | A | C | |
| ATOM | 252 | O | GLU | A | 576 | 50.771 | 62.976 | −13.518 | 1.00 | 35.52 | A | O | |
| ATOM | 253 | N | PHE | A | 577 | 48.800 | 62.097 | −12.864 | 1.00 | 32.97 | A | N | |
| ATOM | 254 | CA | PHE | A | 577 | 48.430 | 61.560 | −14.172 | 1.00 | 37.81 | A | C | |
| ATOM | 255 | CB | PHE | A | 577 | 48.653 | 60.044 | −14.195 | 1.00 | 38.19 | A | C | |
| ATOM | 256 | CG | PHE | A | 577 | 48.255 | 59.385 | −15.488 | 1.00 | 36.08 | A | C | |
| ATOM | 257 | CD1 | PHE | A | 577 | 49.061 | 59.483 | −16.613 | 1.00 | 36.75 | A | C | |
| ATOM | 258 | CD2 | PHE | A | 577 | 47.077 | 58.658 | −15.577 | 1.00 | 35.23 | A | C | |
| ATOM | 259 | CE1 | PHE | A | 577 | 48.698 | 58.866 | −17.802 | 1.00 | 35.50 | A | C | |
| ATOM | 260 | CE2 | PHE | A | 577 | 46.709 | 58.040 | −16.762 | 1.00 | 31.27 | A | C | |
| ATOM | 261 | CZ | PHE | A | 577 | 47.522 | 58.145 | −17.874 | 1.00 | 29.63 | A | C | |
| ATOM | 262 | C | PHE | A | 577 | 46.953 | 61.857 | −14.435 | 1.00 | 39.50 | A | C | |
| ATOM | 263 | O | PHE | A | 577 | 46.127 | 61.786 | −13.524 | 1.00 | 37.92 | A | O | |
| ATOM | 264 | N | PRO | A | 578 | 46.607 | 62.194 | −15.689 | 1.00 | 41.54 | A | N | |
| ATOM | 265 | CD | PRO | A | 578 | 47.551 | 62.385 | −16.805 | 1.00 | 43.08 | A | C | |
| ATOM | 266 | CA | PRO | A | 578 | 45.240 | 62.510 | −16.112 | 1.00 | 42.13 | A | C | |
| ATOM | 267 | CE | PRO | A | 578 | 45.377 | 62.658 | −17.621 | 1.00 | 46.24 | A | C | |
| ATOM | 268 | CG | PRO | A | 578 | 46.745 | 63.228 | −17.766 | 1.00 | 46.41 | A | C | |
| ATOM | 269 | C | PRO | A | 578 | 44.215 | 61.447 | −15.731 | 1.00 | 42.52 | A | C | |
| ATOM | 270 | O | PRO | A | 578 | 44.140 | 60.387 | −16.352 | 1.00 | 44.49 | A | O | |
| ATOM | 271 | N | ARG | A | 579 | 43.425 | 61.750 | −14.709 | 1.00 | 39.50 | A | N | |
| ATOM | 272 | CA | ARG | A | 579 | 42.386 | 60.857 | −14.218 | 1.00 | 42.13 | A | C | |
| ATOM | 273 | CE | ARG | A | 579 | 41.571 | 61.593 | −13.150 | 1.00 | 43.43 | A | C | |
| ATOM | 274 | CG | ARG | A | 579 | 40.348 | 60.865 | −12.630 | 1.00 | 39.15 | A | C | |
| ATOM | 275 | CD | ARG | A | 579 | 39.582 | 61.775 | −11.688 | 1.00 | 33.24 | A | C | |
| ATOM | 276 | NE | ARG | A | 579 | 40.404 | 62.208 | −10.559 | 1.00 | 34.71 | A | N | |
| ATOM | 277 | CZ | ARG | A | 579 | 40.582 | 61.502 | −9.444 | 1.00 | 37.36 | A | C | |
| ATOM | 278 | NH1 | ARG | A | 579 | 39.993 | 60.322 | −9.295 | 1.00 | 31.90 | A | N | |
| ATOM | 279 | NH2 | ARG | A | 579 | 41.355 | 61.976 | −8.476 | 1.00 | 38.21 | A | N | |
| ATOM | 280 | C | ARG | A | 579 | 41.470 | 60.375 | −15.348 | 1.00 | 47.76 | A | C | |
| ATOM | 281 | 0 | ARG | A | 579 | 40.787 | 59.361 | −15.215 | 1.00 | 52.44 | A | O | |
| ATOM | 282 | N | ASN | A | 580 | 41.468 | 61.106 | −16.460 | 1.00 | 53.12 | A | N | |
| ATOM | 283 | CA | ASN | A | 580 | 40.638 | 60.771 | −17.615 | 1.00 | 56.04 | A | C | |
| ATOM | 284 | CE | ASN | A | 580 | 40.321 | 62.040 | −18.413 | 1.00 | 59.98 | A | C | |
| ATOM | 285 | CG | ASN | A | 580 | 39.974 | 61.749 | −19.865 | 1.00 | 66.26 | A | C | |
| ATOM | 286 | OD1 | ASN | A | 580 | 40.814 | 61.284 | −20.635 | 1.00 | 71.83 | A | O | |
| ATOM | 287 | ND2 | ASN | A | 580 | 38.731 | 62.023 | −20.244 | 1.00 | 7.081 | A | N | |
| ATOM | 288 | CA | ASN | A | 580 | 41.259 | 59.731 | −18.543 | 1.00 | 57.00 | A | C | |
| ATOM | 289 | O | ASN | A | 580 | 40.558 | 58.862 | −19.061 | 1.00 | 60.31 | A | O | |
| ATOM | 290 | N | ASN | A | 581 | 42.568 | 59.829 | −18.763 | 1.00 | 54.46 | A | N | |
| ATOM | 291 | CA | ASN | A | 581 | 43.274 | 58.898 | −19.639 | 1.00 | 51.93 | A | C | |
| ATOM | 292 | CB | ASN | A | 581 | 44.673 | 59.429 | −19.963 | 1.00 | 53.14 | A | C | |
| ATOM | 293 | CG | ASN | A | 581 | 44.638 | 60.753 | −20.698 | 1.00 | 51.63 | A | C | |
| ATOM | 294 | OD1 | ASN | A | 581 | 44.053 | 61.721 | −20.222 | 1.00 | 51.37 | A | O | |
| ATOM | 295 | ND2 | ASN | A | 581 | 45.271 | 60.800 | −21.865 | 1.00 | 54.71 | A | N | |
| ATOM | 296 | C | ASN | A | 581 | 43.394 | 57.522 | −18.999 | 1.00 | 53.02 | A | C | |
| ATOM | 297 | O | ASN | A | 581 | 44.361 | 56.796 | −19.240 | 1.00 | 48.84 | A | O | |
| ATOM | 298 | N | LEU | A | 582 | 42.400 | 57.168 | −18.192 | 1.00 | 51.03 | A | N | |
| ATOM | 299 | CA | LEU | A | 582 | 42.389 | 55.888 | −17.504 | 1.00 | 54.48 | A | C | |
| ATOM | 300 | CB | LEU | A | 582 | 42.708 | 56.111 | −16.022 | 1.00 | 56.26 | A | C | |
| ATOM | 301 | CG | LEU | A | 582 | 43.522 | 55.077 | −15.242 | 1.00 | 53.82 | A | C | |
| ATOM | 302 | CD1 | LEU | A | 582 | 44.896 | 54.919 | −15.873 | 1.00 | 51.83 | A | C | |
| ATOM | 303 | CD2 | LEU | A | 582 | 43.657 | 55.533 | −13.797 | 1.00 | 52.98 | A | C | |
| ATOM | 304 | C | LEU | A | 582 | 41.009 | 55.245 | −17.647 | 1.00 | 57.15 | A | C | |
| ATOM | 305 | O | LEU | A | 582 | 40.010 | 55.812 | −17.206 | 1.00 | 59.35 | A | O | |
| ATOM | 306 | N | GLN | A | 583 | 40.949 | 54.074 | −18.276 | 1.00 | 56.30 | A | N | |
| ATOM | 307 | CA | GLN | A | 583 | 39.678 | 53.371 | −18.438 | 1.00 | 55.57 | A | C | |
| ATOM | 308 | CB | GLN | A | 583 | 39.479 | 52.898 | −19.878 | 1.00 | 60.58 | A | C | |
| ATOM | 309 | CG | GLN | A | 583 | 38.172 | 52.135 | −20.072 | 1.00 | 65.90 | A | C | |
| ATOM | 310 | CD | GLN | A | 583 | 38.113 | 51.387 | −21.387 | 1.00 | 71.23 | A | C | |
| ATOM | 311 | OE1 | GLN | A | 583 | 38.208 | 51.984 | −22.460 | 1.00 | 75.78 | A | O | |
| ATOM | 312 | NE2 | GLN | A | 583 | 37.958 | 50.069 | −21.312 | 1.00 | 75.45 | A | N | |
| ATOM | 313 | C | GLN | A | 583 | 39.668 | 52.160 | −17.524 | 1.00 | 51.30 | A | C | |
| ATOM | 314 | O | GLN | A | 583 | 40.377 | 51.188 | −17.773 | 1.00 | 48.34 | A | O | |
| ATOM | 315 | N | PHE | A | 584 | 38.858 | 52.214 | −16.473 | 1.00 | 49.58 | A | N | |
| ATOM | 316 | CA | PHE | A | 584 | 38.786 | 51.112 | −15.526 | 1.00 | 50.64 | A | C | |
| ATOM | 317 | CB | PHE | A | 584 | 37.896 | 51.480 | −14.335 | 1.00 | 53.40 | A | C | |
| ATOM | 318 | CG | PHE | A | 584 | 38.482 | 52.542 | −13.444 | 1.00 | 53.06 | A | C | |
| ATOM | 319 | CD1 | PHE | A | 584 | 39.845 | 52.800 | −13.454 | 1.00 | 53.35 | A | C | |
| ATOM | 320 | CD2 | PHE | A | 584 | 37.674 | 53.261 | −12.575 | 1.00 | 52.65 | A | C | |
| ATOM | 321 | CE1 | PHE | A | 584 | 40.394 | 53.755 | −12.614 | 1.00 | 51.51 | A | C | |
| ATOM | 322 | CE2 | PHE | A | 584 | 38.216 | 54.216 | −11.731 | 1.00 | 52.15 | A | C | |
| ATOM | 323 | CZ | PHE | A | 584 | 39.580 | 54.463 | −11.751 | 1.00 | 50.37 | A | C | |
| ATOM | 324 | C | PHE | A | 584 | 38.293 | 49.811 | −16.140 | 1.00 | 52.87 | A | C | |
| ATOM | 325 | O | PHE | A | 584 | 37.316 | 49.788 | −16.887 | 1.00 | 52.62 | A | O | |
| ATOM | 326 | N | GLY | A | 585 | 38.987 | 48.726 | −15.813 | 1.00 | 51.95 | A | N | |
| ATOM | 327 | CA | GLY | A | 585 | 38.614 | 47.418 | −16.314 | 1.00 | 46.36 | A | C | |
| ATOM | 328 | C | GLY | A | 585 | 38.134 | 46.526 | −15.183 | 1.00 | 42.19 | A | C | |
| ATOM | 329 | O | GLY | A | 585 | 37.433 | 46.978 | −14.277 | 1.00 | 41.40 | A | O | |
| ATOM | 330 | N | LYS | A | 586 | 38.525 | 45.258 | −15.230 | 1.00 | 43.47 | A | N | |
| ATOM | 331 | CA | LYS | A | 586 | 38.134 | 44.283 | −14.218 | 1.00 | 45.09 | A | C | |
| ATOM | 332 | CB | LYS | A | 586 | 38.492 | 42.874 | −14.696 | 1.00 | 44.79 | A | C | |
| ATOM | 333 | CG | LYS | A | 586 | 39.972 | 42.700 | −15.001 | 1.00 | 54.34 | A | C | |
| ATOM | 334 | CD | LYS | A | 586 | 40.310 | 41.277 | −15.422 | 1.00 | 54.93 | A | C | |
| ATOM | 335 | CE | LYS | A | 586 | 40.094 | 40.292 | −14.287 | 1.00 | 54.08 | A | C | |
| ATOM | 336 | NZ | LYS | A | 586 | 40.444 | 38.903 | −14.705 | 1.00 | 53.34 | A | N | |
| ATOM | 337 | C | LYS | A | 586 | 38.790 | 44.532 | −12.860 | 1.00 | 44.37 | A | C | |
| ATOM | 338 | O | LYS | A | 586 | 39.898 | 45.068 | −12.782 | 1.00 | 39.89 | A | O | |
| ATOM | 339 | N | THR | A | 587 | 38.095 | 44.129 | −11.799 | 1.00 | 42.38 | A | N | |
| ATOM | 340 | CA | THR | A | 587 | 38.582 | 44.272 | −10.430 | 1.00 | 39.75 | A | C | |
| ATOM | 341 | CB | THR | A | 587 | 37.417 | 44.270 | −9.425 | 1.00 | 38.20 | A | C | |
| ATOM | 342 | OG1 | THR | A | 587 | 36.617 | 45.441 | −9.612 | 1.00 | 41.55 | A | O | |
| ATOM | 343 | CG2 | THR | A | 587 | 37.943 | 44.239 | −7.995 | 1.00 | 44.74 | A | C | |
| ATOM | 344 | C | THR | A | 587 | 39.509 | 43.103 | −10.081 | 1.00 | 41.73 | A | C | |
| ATOM | 345 | O | THR | A | 587 | 39.049 | 41.992 | −9.827 | 1.00 | 43.31 | A | O | |
| ATOM | 346 | N | LEU | A | 588 | 40.812 | 43.359 | −10.070 | 1.00 | 39.85 | A | N | |
| ATOM | 347 | CA | LEU | A | 588 | 41.798 | 42.331 | −9.756 | 1.00 | 33.01 | A | C | |
| ATOM | 348 | CB | LEU | A | 588 | 43.206 | 42.919 | −9.852 | 1.00 | 35.74 | A | C | |
| ATOM | 349 | CG | LEU | A | 588 | 43.885 | 42.782 | −11.217 | 1.00 | 45.14 | A | C | |
| ATOM | 350 | CD1 | LEU | A | 588 | 42.840 | 42.777 | −12.326 | 1.00 | 42.62 | A | C | |
| ATOM | 351 | CD2 | LEU | A | 588 | 44.901 | 43.907 | −11.390 | 1.00 | 39.42 | A | C | |
| ATOM | 352 | C | LEU | A | 588 | 41.598 | 41.715 | −8.379 | 1.00 | 35.37 | A | C | |
| ATOM | 353 | O | LEU | A | 588 | 41.812 | 40.517 | 8.186 | 1.00 | 36.10 | A | O | |
| ATOM | 354 | N | GLY | A | 589 | 41.196 | 42.538 | −7.420 | 1.00 | 34.55 | A | N | |
| ATOM | 355 | CA | GLY | A | 589 | 40.980 | 42.049 | −6.070 | 1.00 | 29.06 | A | C | |
| ATOM | 356 | C | GLY | A | 589 | 40.468 | 43.180 | −5.205 | 1.00 | 30.79 | A | C | |
| ATOM | 357 | O | GLY | A | 589 | 40.649 | 44.351 | −5.545 | 1.00 | 33.83 | A | O | |
| ATOM | 358 | N | ALA | A | 590 | 39.837 | 42.856 | −4.085 | 1.00 | 24.47 | A | N | |
| ATOM | 359 | CA | ALA | A | 590 | 39.308 | 43.905 | −3.227 | 1.00 | 27.44 | A | C | |
| ATOM | 360 | CB | ALA | A | 590 | 37.853 | 44.213 | −3.612 | 1.00 | 31.60 | A | C | |
| ATOM | 361 | C | ALA | A | 590 | 39.388 | 43.532 | −1.766 | 1.00 | 24.67 | A | C | |
| ATOM | 362 | O | ALA | A | 590 | 39.327 | 42.357 | −1.418 | 1.00 | 31.10 | A | O | |
| ATOM | 363 | N | GLY | A | 591 | 39.517 | 44.543 | −0.913 | 1.00 | 29.82 | A | N | |
| ATOM | 364 | CA | GLY | A | 591 | 39.608 | 44.301 | 0.514 | 1.00 | 39.85 | A | C | |
| ATOM | 365 | C | GLY | A | 591 | 38.584 | 45.069 | 1.324 | 1.00 | 45.53 | A | C | |
| ATOM | 366 | O | GLY | A | 591 | 37.540 | 45.475 | 0.811 | 1.00 | 43.66 | A | 0 | |
| ATOM | 367 | N | ALA | A | 592 | 38.892 | 45.277 | 2.599 | 1.00 | 49.91 | A | N | |
| ATOM | 368 | CA | ALA | A | 592 | 37.996 | 45.987 | 3.500 | 1.00 | 51.25 | A | C | |
| ATOM | 369 | CB | ALA | A | 592 | 38.403 | 45.718 | 4.942 | 1.00 | 51.95 | A | C | |
| ATOM | 370 | C | ALA | A | 592 | 37.954 | 47.490 | 3.251 | 1.00 | 54.77 | A | C | |
| ATOM | 371 | O | ALA | A | 592 | 36.875 | 48.089 | 3.210 | 1.00 | 56.08 | A | O | |
| ATOM | 372 | N | PHE | A | 593 | 39.127 | 48.094 | 3.083 | 1.00 | 55.02 | A | N | |
| ATOM | 373 | CA | PHE | A | 593 | 39.224 | 49.536 | 2.872 | 1.00 | 52.35 | A | C | |
| ATOM | 374 | CB | PHE | A | 593 | 40.483 | 50.080 | 3.550 | 1.00 | 57.34 | A | C | |
| ATOM | 375 | CG | PHE | A | 593 | 40.806 | 49.417 | 4.857 | 1.00 | 64.78 | A | C | |
| ATOM | 376 | CD1 | PHE | A | 593 | 39.891 | 49.412 | 5.896 | 1.00 | 68.19 | A | C | |
| ATOM | 377 | CD2 | PHE | A | 593 | 42.037 | 48.801 | 5.046 | 1.00 | 69.62 | A | C | |
| ATOM | 378 | CE1 | PHE | A | 593 | 40.196 | 48.803 | 7.103 | 1.00 | 73.09 | A | C | |
| ATOM | 379 | CE2 | PHE | A | 593 | 42.349 | 48.190 | 6.250 | 1.00 | 70.39 | A | C | |
| ATOM | 380 | CZ | PHE | A | 593 | 41.428 | 48.191 | 7.280 | 1.00 | 73.37 | A | C | |
| ATOM | 381 | C | PHE | A | 593 | 39.238 | 49.972 | 1.408 | 1.00 | 47.62 | A | C | |
| ATOM | 382 | O | PHE | A | 593 | 38.888 | 51.109 | 1.099 | 1.00 | 50.90 | A | O | |
| ATOM | 383 | N | GLY | A | 594 | 39.647 | 49.086 | 0.506 | 1.00 | 41.94 | A | N | |
| ATOM | 384 | CA | GLY | A | 594 | 39.695 | 49.468 | 0.893 | 1.00 | 29.97 | A | C | |
| ATOM | 385 | C | GLY | A | 594 | 39.797 | 48.328 | −1.884 | 1.00 | 32.94 | A | C | |
| ATOM | 386 | O | GLY | A | 594 | 39.461 | 47.186 | 1.579 | 1.00 | 37.39 | A | O | |
| ATOM | 387 | N | LYS | A | 595 | 40.270 | 48.641 | −3.083 | 1.00 | 31.08 | A | N | |
| ATOM | 388 | CA | LYS | A | 595 | 40.400 | 47.640 | 4.123 | 1.00 | 30.13 | A | C | |
| ATOM | 389 | CB | LYS | A | 595 | 39.036 | 47.406 | 4.774 | 1.00 | 35.57 | A | C | |
| ATOM | 390 | CG | LYS | A | 595 | 38.360 | 48.696 | −5.216 | 1.00 | 40.48 | A | C | |
| ATOM | 391 | CD | LYS | A | 595 | 36.880 | 48.477 | 5.510 | 1.00 | 51.46 | A | C | |
| ATOM | 392 | CE | LYS | A | 595 | 36.132 | 49.801 | −5.585 | 1.00 | 52.40 | A | C | |
| ATOM | 393 | NZ | LYS | A | 595 | 34.655 | 49.603 | −5.611 | 1.00 | 53.83 | A | N | |
| ATOM | 394 | C | LYS | A | 595 | 41.393 | 48.087 | −5.178 | 1.00 | 30.80 | A | C | |
| ATOM | 395 | O | LYS | A | 595 | 41.830 | 49.237 | 5.189 | 1.00 | 29.83 | A | O | |
| ATOM | 396 | N | VAL | A | 596 | 41.749 | 47.160 | −6.056 | 1.00 | 24.54 | A | N | |
| ATOM | 397 | CA | VAL | A | 596 | 42.671 | 47.435 | 7.143 | 1.00 | 32.04 | A | C | |
| ATOM | 398 | CB | VAL | A | 596 | 44.054 | 46.743 | 6.923 | 1.00 | 35.30 | A | C | |
| ATOM | 399 | CG1 | VAL | A | 596 | 43.890 | 45.219 | 6.835 | 1.00 | 29.59 | A | C | |
| ATOM | 400 | CG2 | VAL | A | 596 | 45.006 | 47.123 | 8.053 | 1.00 | 28.30 | A | C | |
| ATOM | 401 | C | VAL | A | 596 | 42.013 | 46.915 | −8.417 | 1.00 | 31.88 | A | C | |
| ATOM | 402 | O | VAL | A | 596 | 41.499 | 45.794 | 8.454 | 1.00 | 35.69 | A | O | |
| ATOM | 403 | N | VAL | A | 597 | 42.014 | 47.733 | 9.460 | 1.00 | 33.53 | A | N | |
| ATOM | 404 | CA | VAL | A | 597 | 41.392 | 47.332 | −10.707 | 1.00 | 33.61 | A | C | |
| ATOM | 405 | CB | VAL | A | 597 | 40.200 | 48.243 | −11.041 | 1.00 | 37.26 | A | C | |
| ATOM | 406 | CQ1 | VAL | A | 597 | 39.259 | 48.323 | −9.849 | 1.00 | 35.70 | A | C | |
| ATOM | 407 | CG2 | VAL | A | 597 | 40.698 | 49.622 | −11.415 | 1.00 | 39.07 | A | C | |
| ATOM | 408 | C | VAL | A | 597 | 42.374 | 47.393 | −11.858 | 1.00 | 35.53 | A | C | |
| ATOM | 409 | O | VAL | A | 597 | 43.361 | 48.130 | −11.810 | 1.00 | 30.90 | A | O | |
| ATOM | 410 | N | GLU | A | 598 | 42.110 | 46.600 | −12.887 | 1.00 | 32.63 | A | N | |
| ATOM | 411 | CA | GLU | A | 598 | 42.959 | 46.606 | −14.060 | 1.00 | 36.26 | A | C | |
| ATOM | 412 | CB | GLU | A | 598 | 42.822 | 45.294 | −14.832 | 1.00 | 39.08 | A | C | |
| ATOM | 413 | CG | GLU | A | 598 | 43.547 | 45.285 | −16.171 | 1.00 | 43.81 | A | C | |
| ATOM | 414 | CD | GLU | A | 598 | 43.638 | 43.896 | −16.778 | 1.00 | 44.32 | A | C | |
| ATOM | 415 | OE1 | GLU | A | 598 | 42.716 | 43.084 | −16.555 | 1.00 | 41.77 | A | O | |
| ATOM | 416 | OE2 | GLU | A | 598 | 44.628 | 43.621 | −17.489 | 1.00 | 44.58 | A | O | |
| ATOM | 417 | C | GLU | A | 598 | 42.468 | 47.774 | −14.901 | 1.00 | 37.63 | A | C | |
| ATOM | 418 | O | GLU | A | 598 | 41.323 | 48.199 | −14.764 | 1.00 | 40.80 | A | O | |
| ATOM | 419 | N | ALA | A | 599 | 43.328 | 48.306 | −15.757 | 1.00 | 36.17 | A | N | |
| ATOM | 420 | CA | ALA | A | 599 | 42.927 | 49.431 | −16.582 | 1.00 | 38.98 | A | C | |
| ATOM | 421 | CB | ALA | A | 599 | 42.743 | 50.677 | −15.717 | 1.00 | 34.99 | A | C | |
| ATOM | 422 | C | ALA | A | 599 | 43.943 | 49.703 | −17.667 | 1.00 | 39.88 | A | C | |
| ATOM | 423 | O | ALA | A | 599 | 45.060 | 49.173 | −17.657 | 1.00 | 32.51 | A | O | |
| ATOM | 424 | N | THR | A | 600 | 43.540 | 50.534 | −18.616 | 1.00 | 44.21 | A | N | |
| ATOM | 425 | CA | THR | A | 600 | 44.414 | 50.894 | −19.711 | 1.00 | 47.57 | A | C | |
| ATOM | 426 | CB | THR | A | 600 | 43.714 | 50.678 | −21.054 | 1.00 | 51.77 | A | C | |
| ATOM | 427 | OG1 | THR | A | 600 | 42.900 | 49.499 | −20.979 | 1.00 | 51.44 | A | O | |
| ATOM | 428 | CG2 | THR | A | 600 | 44.741 | 50.494 | −22.157 | 1.00 | 56.65 | A | C | |
| ATOM | 429 | C | THR | A | 600 | 44.756 | 52.365 | −19.538 | 1.00 | 47.69 | A | C | |
| ATOM | 430 | O | THR | A | 600 | 43.869 | 53.206 | −19.378 | 1.00 | 51.13 | A | O | |
| ATOM | 431 | N | ALA | A | 601 | 46.046 | 52.668 | −19.542 | 1.00 | 47.94 | A | N | |
| ATOM | 432 | CA | ALA | A | 601 | 46.507 | 54.036 | −19.382 | 1.00 | 49.59 | A | C | |
| ATOM | 433 | CB | ALA | A | 601 | 47.656 | 54.085 | −18.386 | 1.00 | 49.60 | A | C | |
| ATOM | 434 | C | ALA | A | 601 | 46.957 | 54.566 | −20.733 | 1.00 | 48.79 | A | C | |
| ATOM | 435 | O | ALA | A | 601 | 47.899 | 54.046 | −21.331 | 1.00 | 45.73 | A | O | |
| ATOM | 436 | N | PHE | A | 602 | 46.279 | 55.601 | −21.213 | 1.00 | 50.28 | A | N | |
| ATOM | 437 | CA | PHE | A | 602 | 46.621 | 56.181 | −22.500 | 1.00 | 52.84 | A | C | |
| ATOM | 438 | CB | PHE | A | 602 | 45.356 | 56.669 | −23.209 | 1.00 | 51.88 | A | C | |
| ATOM | 439 | CG | PHE | A | 602 | 44.176 | 55.754 | −23.037 | 1.00 | 51.31 | A | C | |
| ATOM | 440 | CD1 | PHE | A | 602 | 43.295 | 55.929 | −21.978 | 1.00 | 48.72 | A | C | |
| ATOM | 441 | CD2 | PHE | A | 602 | 43.959 | 54.705 | −23.914 | 1.00 | 48.49 | A | C | |
| ATOM | 442 | CE1 | PHE | A | 602 | 42.218 | 55.072 | −21.798 | 1.00 | 47.53 | A | C | |
| ATOM | 443 | CE2 | PHE | A | 602 | 42.884 | 53.844 | −23.739 | 1.00 | 53.37 | A | C | |
| ATOM | 444 | CZ | PHE | A | 602 | 42.011 | 54.029 | −22.678 | 1.00 | 46.43 | A | C | |
| ATOM | 445 | C | PHE | A | 602 | 47.597 | 57.332 | −22.310 | 1.00 | 52.01 | A | C | |
| ATOM | 446 | O | PHE | A | 602 | 47.279 | 58.332 | −21.668 | 1.00 | 51.47 | A | O | |
| ATOM | 447 | N | GLY | A | 603 | 48.792 | 57.175 | −22.866 | 1.00 | 51.12 | A | N | |
| ATOM | 448 | CA | GLY | A | 603 | 49.802 | 58.208 | −22.752 | 1.00 | 54.60 | A | C | |
| ATOM | 449 | C | GLY | A | 603 | 50.385 | 58.299 | −21.357 | 1.00 | 55.28 | A | C | |
| ATOM | 450 | O | GLY | A | 603 | 50.481 | 59.388 | −20.790 | 1.00 | 57.85 | A | O | |
| ATOM | 451 | N | LEU | A | 604 | 502779 | 57.155 | −20.804 | 1.00 | 55.98 | A | N | |
| ATOM | 452 | CA | LEU | A | 604 | 51.352 | 57.119 | −19.464 | 1.00 | 54.42 | A | C | |
| ATOM | 453 | CB | LEU | A | 604 | 50.856 | 55.885 | −18.707 | 1.00 | 52.63 | A | C | |
| ATOM | 454 | CG | LEU | A | 604 | 50.668 | 56.029 | −17.190 | 1.00 | 53.02 | A | C | |
| ATOM | 455 | CD1 | LEU | A | 604 | 50.348 | 54.669 | −16.589 | 1.00 | 47.74 | A | C | |
| ATOM | 456 | CD2 | LEU | A | 604 | 51.914 | 56.609 | −16.553 | 1.00 | 45.06 | A | C | |
| ATOM | 457 | C | LEU | A | 604 | 52.869 | 57.085 | −19.553 | 1.00 | 57.25 | A | C | |
| ATOM | 458 | O | LEU | A | 604 | 53.439 | 56.279 | −20.288 | 1.00 | 59.13 | A | O | |
| ATOM | 459 | N | GLY | A | 605 | 53.520 | 57.965 | −18.801 | 1.00 | 59.81 | A | N | |
| ATOM | 460 | CA | GLY | A | 605 | 54.969 | 58.013 | −18.812 | 1.00 | 61.32 | A | C | |
| ATOM | 461 | C | GLY | A | 605 | 55.501 | 58.603 | −20.100 | 1.00 | 64.09 | A | C | |
| ATOM | 462 | O | GLY | A | 605 | 54.742 | 58.858 | −21.037 | 1.00 | 64.12 | A | O | |
| ATOM | 463 | N | LYS | A | 606 | 56.809 | 58.824 | −20.145 | 1.00 | 67.16 | A | N | |
| ATOM | 464 | CA | LYS | A | 606 | 57.447 | 59.387 | −21.325 | 1.00 | 73.06 | A | C | |
| ATOM | 465 | CB | LYS | A | 606 | 58.967 | 59.266 | −21.194 | 1.00 | 74.86 | A | C | |
| ATOM | 466 | CC | LYS | A | 606 | 59.499 | 60.029 | −19.986 | 1.00 | 77.56 | A | C | |
| ATOM | 467 | CD | LYS | A | 606 | 60.981 | 59.814 | −19.743 | 1.00 | 78.95 | A | C | |
| ATOM | 468 | CE | LYS | A | 606 | 61.414 | 60.538 | −18.472 | 1.00 | 81.67 | A | C | |
| ATOM | 469 | NZ | LYS | A | 606 | 62.860 | 60.359 | −18.164 | 1.00 | 84.93 | A | N | |
| ATOM | 470 | C | LYS | A | 606 | 56.938 | 58.654 | −22.559 | 1.00 | 75.50 | A | C | |
| ATOM | 471 | O | LYS | A | 606 | 56.313 | 59.255 | −23.433 | 1.00 | 75.14 | A | O | |
| ATOM | 472 | N | GLU | A | 607 | 5.7.194 | 57.354 | −22.625 | 1.00 | 78.26 | A | N | |
| ATOM | 473 | CA | GLU | A | 607 | 56.716 | 56.567 | −23.749 | 1.00 | 81.93 | A | C | |
| ATOM | 474 | CB | GLU | A | 607 | 57.274 | 55.145 | −23.684 | 1.00 | 84.74 | A | C | |
| ATOM | 475 | CG | GLU | A | 607 | 58.744 | 55.084 | −23.310 | 1.00 | 86.75 | A | C | |
| ATOM | 476 | CD | GLU | A | 607 | 59.569 | 56.126 | −24.037 | 1.00 | 90.39 | A | C | |
| ATOM | 477 | OE1 | GLU | A | 607 | 59.611 | 56.094 | −25.286 | 1.00 | 92.77 | A | O | |
| ATOM | 478 | OE2 | GLU | A | 607 | 60.173 | 56.981 | −23.3.55 | 1.00 | 91.60 | A | O | |
| ATOM | 479 | C | GLU | A | 607 | 55.201 | 56.545 | −23.588 | 1.00 | 83.47 | A | C | |
| ATOM | 480 | O | GLU | A | 607 | 54.671 | 55.833 | −22.734 | 1.00 | 85.59 | A | O | |
| ATOM | 481 | N | ASP | A | 608 | 54.510 | 57.339 | −24.399 | 1.00 | 81.98 | A | N | |
| ATOM | 482 | CA | ASP | A | 608 | 53.055 | 57.434 | −24.333 | 1.00 | 80.62 | A | C | |
| ATOM | 483 | CB | ASP | A | 608 | 52.560 | 58.507 | −25.304 | 1.00 | 82.58 | A | C | |
| ATOM | 484 | CG | ASP | A | 608 | 53.190 | 59.858 | −25.043 | 1.00 | 84.79 | A | C | |
| ATOM | 485 | OD1 | ASP | A | 608 | 54.418 | 59.987 | −25.235 | 1.00 | 86.74 | A | O | |
| ATOM | 486 | OD2 | ASP | A | 608 | 52.460 | 60.788 | −24.639 | 1.00 | 87.45 | A | O | |
| ATOM | 487 | C | ASP | A | 608 | 52.336 | 56.121 | −24.619 | 1.00 | 77.60 | A | C | |
| ATOM | 488 | O | ASP | A | 608 | 51.170 | 56.120 | −25.014 | 1.00 | 78.28 | A | O | |
| ATOM | 489 | N | ALA | A | 609 | 53.028 | 55.007 | −24.414 | 1.00 | 75.42 | A | N | |
| ATOM | 490 | CA | ALA | A | 609 | 52.443 | 53.694 | −24.649 | 1.00 | 72.97 | A | C | |
| ATOM | 491 | CB | ALA | A | 609 | 53.411 | 52.602 | −24.202 | 1.00 | 75.62 | A | C | |
| ATOM | 492 | C | ALA | A | 609 | 51.120 | 53.559 | −23.905 | 1.00 | 70.13 | A | C | |
| ATOM | 493 | O | ALA | A | 609 | 50.899 | 54.211 | −22.881 | 1.00 | 66.39 | A | O | |
| ATOM | 494 | N | VAL | A | 610 | 50.241 | 52.714 | −24.433 | 1.00 | 68.09 | A | N | |
| ATOM | 495 | CA | VAL | A | 610 | 48.934 | 52.477 | −23.830 | 1.00 | 64.25 | A | C | |
| ATOM | 496 | CB | VAL | A | 610 | 47.826 | 52.418 | −24.913 | 1.00 | 59.85 | A | C | |
| ATOM | 497 | CG1 | VAL | A | 610 | 46.455 | 52.349 | −24.262 | 1.00 | 56.84 | A | C | |
| ATOM | 498 | CG2 | VAL | A | 610 | 47.922 | 53.636 | −25.819 | 1.00 | 54.82 | A | C | |
| ATOM | 499 | C | VAL | A | 610 | 48.983 | 51.148 | −23.079 | 1.00 | 62.29 | A | C | |
| ATOM | 500 | O | VAL | A | 610 | 48.181 | 50.247 | −23.329 | 1.00 | 63.16 | A | O | |
| ATOM | 501 | N | LEU | A | 611 | 49.933 | 51.038 | −22.155 | 1.00 | 59.06 | A | N | |
| ATOM | 502 | CA | LEU | A | 611 | 50.108 | 49.821 | −21.371 | 1.00 | 57.74 | A | C | |
| ATOM | 503 | CB | LEU | A | 611 | 51.475 | 49.840 | −20.679 | 1.00 | 58.65 | A | C | |
| ATOM | 504 | CG | LEU | A | 611 | 51.968 | 51.173 | −20.107 | 1.00 | 61.98 | A | C | |
| ATOM | 505 | CD1 | LEU | A | 611 | 50.925 | 51.766 | −19.185 | 1.00 | 62.53 | A | C | |
| ATOM | 506 | CD2 | LEU | A | 611 | 53.278 | 50.952 | −19.366 | 1.00 | 62.46 | A | C | |
| ATOM | 507 | C | LEU | A | 611 | 49.014 | 49.543 | −20.342 | 1.00 | 53.57 | A | C | |
| ATOM | 508 | O | LEU | A | 611 | 48.339 | 50.452 | −19.859 | 1.00 | 52.15 | A | O | |
| ATOM | 509 | N | LYS | A | 612 | 48.847 | 48.263 | −20.024 | 1.00 | 54.36 | A | N | |
| ATOM | 510 | CA | LYS | A | 612 | 47.859 | 47.821 | −19.049 | 1.00 | 51.58 | A | C | |
| ATOM | 511 | CB | LYS | A | 612 | 47.608 | 46.317 | −19.198 | 1.00 | 54.96 | A | C | |
| ATOM | 512 | CG | LYS | A | 612 | 46.371 | 45.809 | −18.474 | 1.00 | 58.00 | A | C | |
| ATOM | 513 | CD | LYS | A | 612 | 45.174 | 45.707 | −19.413 | 1.00 | 66.68 | A | C | |
| ATOM | 514 | CE | LYS | A | 612 | 44.813 | 47.046 | −20.038 | 1.00 | 71.12 | A | C | |
| ATOM | 515 | NZ | LYS | A | 612 | 43.678 | 46.915 | −20.996 | 1.00 | 70.54 | A | N | |
| ATOM | 516 | C | LYS | A | 612 | 48.450 | 48.113 | −17.675 | 1.00 | 45.89 | A | C | |
| ATOM | 517 | O | LYS | A | 612 | 49.650 | 47.937 | −17.465 | 1.00 | 42.47 | A | O | |
| ATOM | 518 | N | VAL | A | 613 | 47.615 | 48.562 | −16.743 | 1.00 | 38.49 | A | N | |
| ATOM | 519 | CA | VAL | A | 613 | 48.094 | 48.894 | −15.407 | 1.00 | 34.26 | A | C | |
| ATOM | 520 | CB | VAL | A | 613 | 48.392 | 50.415 | −15.269 | 1.00 | 27.93 | A | C | |
| ATOM | 521 | CG1 | VAL | A | 613 | 49.346 | 50.864 | −16.363 | 1.00 | 30.91 | A | C | |
| ATOM | 522 | CG2 | VAL | A | 613 | 47.101 | 51.215 | −15.330 | 1.00 | 22.94 | A | C | |
| ATOM | 523 | C | VAL | A | 613 | 47.079 | 48.518 | −14.342 | 1.00 | 32.02 | A | C | |
| ATOM | 524 | O | VAL | A | 613 | 45.971 | 48.097 | −14.652 | 1.00 | 28.00 | A | O | |
| ATOM | 525 | N | ALA | A | 614 | 47.482 | 48.672 | −13.084 | 1.00 | 29.17 | A | N | |
| ATOM | 526 | CA | ALA | A | 614 | 46.633 | 48.376 | −11.938 | 1.00 | 29.17 | A | C | |
| ATOM | 527 | CB | ALA | A | 614 | 47.338 | 47.397 | −11.005 | 1.00 | 32.26 | A | C | |
| ATOM | 528 | C | ALA | A | 614 | 46.383 | 49.695 | −11.221 | 1.00 | 30.22 | A | C | |
| ATOM | 529 | O | ALA | A | 614 | 47.289 | 50.518 | −11.105 | 1.00 | 29.40 | A | O | |
| ATOM | 530 | N | VAL | A | 615 | 45.161 | 49.894 | −10.736 | 1.00 | 29.87 | A | N | |
| ATOM | 531 | CA | VAL | A | 615 | 44.798 | 51.133 | −10.061 | 1.00 | 27.61 | A | C | |
| ATOM | 532 | CB | VAL | A | 615 | 43.761 | 51.936 | −10.906 | 1.00 | 31.06 | A | C | |
| ATOM | 533 | CG1 | VAL | A | 615 | 43.505 | 53.297 | −10.283 | 1.00 | 23.19 | A | C | |
| ATOM | 534 | CG2 | VAL | A | 61 | 44.258 | 52.090 | −12.336 | 1.00 | 24.92 | A | C | |
| ATOM | 535 | C | VAL | A | 615 | 44.192 | 50.852 | −8.693 | 1.00 | 29.60 | A | C | |
| ATOM | 536 | O | VAL | A | 615 | 43.131 | 50.243 | −8.596 | 1.00 | 31.31 | A | O | |
| ATOM | 537 | N | LYS | A | 616 | 44.862 | 51.290 | −7.633 | 1.00 | 30.91 | A | N | |
| ATOM | 538 | CA | LYS | A | 616 | 44.332 | 51.075 | −6.295 | 1.00 | 30.66 | A | C | |
| ATOM | 539 | CB | LYS | A | 616 | 45.461 | 50.781 | −5.304 | 1.00 | 35.30 | A | C | |
| ATOM | 540 | CG | LYS | A | 616 | 44.984 | 50.022 | −4.072 | 1.00 | 48.47 | A | C | |
| ATOM | 541 | CD | LYS | A | 616 | 46.133 | 49.518 | −3.210 | 1.00 | 53.24 | A | C | |
| ATOM | 542 | CB | LYS | A | 616 | 46.863 | 50.660 | −2.540 | 1.00 | 53.43 | A | C | |
| ATOM | 543 | NZ | LYS | A | 616 | 45.934 | 51.438 | −1.678 | 1.00 | 58.24 | A | N | |
| ATOM | 544 | C | LYS | A | 616 | 43.554 | 52.323 | −5.870 | 1.00 | 29.05 | A | C | |
| ATOM | 545 | O | LYS | A | 616 | 43.898 | 53.445 | −6.252 | 1.00 | 25.57 | A | O | |
| ATOM | 546 | N | MET | A | 617 | 42.508 | 52.119 | −5.076 | 1.00 | 26.07 | A | N | |
| ATOM | 547 | CA | MET | A | 617 | 41.650 | 53.212 | −4.623 | 1.00 | 34.81 | A | C | |
| ATOM | 548 | CB | MET | A | 617 | 40.637 | 53.549 | 5.715 | 1.00 | 33.43 | A | C | |
| ATOM | 549 | CG | MET | A | 617 | 39.722 | 52.372 | −6.037 | 1.00 | 36.82 | A | C | |
| ATOM | 550 | SD | MET | A | 617 | 38.995 | 52.461 | −7.670 | 1.00 | 38.05 | A | S | |
| ATOM | 551 | CB | MET | A | 617 | 40.387 | 51.929 | −8.680 | 1.00 | 39.70 | A | C | |
| ATOM | 552 | C | MET | A | 617 | 40.899 | 52.793 | −3.367 | 1.00 | 32.33 | A | C | |
| ATOM | 553 | 0 | MET | A | 617 | 40.833 | 51.607 | −3.043 | 1.00 | 37.05 | A | O | |
| ATOM | 554 | N | LEU | A | 618 | 40.317 | 53.767 | −2.676 | 1.00 | 35.77 | A | N | |
| ATOM | 555 | CA | LEU | A | 618 | 39.577 | 53.492 | −1.449 | 1.00 | 37.88 | A | C | |
| ATOM | 556 | CB | LEU | A | 618 | 39.717 | 54.661 | −0.476 | 1.00 | 35.44 | A | C | |
| ATOM | 557 | CG | LEU | A | 618 | 41.125 | 54.928 | 0.049 | 1.00 | 39.79 | A | C | |
| ATOM | 558 | CD1 | LEU | A | 618 | 41.069 | 56.023 | 1.106 | 1.00 | 28.83 | A | C | |
| ATOM | 559 | CD2 | LEU | A | 618 | 41.700 | 53.644 | 0.635 | 1.00 | 39.94 | A | C | |
| ATOM | 560 | C | LEU | A | 618 | 38.094 | 53.215 | −1.666 | 1.00 | 40.94 | A | C | |
| ATOM | 561 | O | LEU | A | 618 | 37.575 | 53.373 | −2.770 | 1.00 | 34.19 | A | O | |
| ATOM | 562 | N | LYS | A | 619 | 37.430 | 52.793 | −0.590 | 1.00 | 44.91 | A | N | |
| ATOM | 563 | CA | LYS | A | 619 | 35.998 | 52.505 | −0.593 | 1.00 | 50.05 | A | C | |
| ATOM | 564 | CB | LYS | A | 619 | 35.724 | 51.089 | −0.082 | 1.00 | 48.11 | A | C | |
| ATOM | 565 | CG | LYS | A | 619 | 36.012 | 49.976 | −1.061 | 1.00 | 50.29 | A | C | |
| ATOM | 566 | CD | LYS | A | 619 | 35.636 | 48.638 | −0.448 | 1.00 | 49.52 | A | C | |
| ATOM | 567 | CB | LYS | A | 619 | 35.773 | 47.508 | −1.448 | 1.00 | 5093 | A | C | |
| ATOM | 568 | NZ | LYS | A | 619 | 35.347 | 46.214 | −0.851 | 1.00 | 55.92 | A | N | |
| ATOM | 569 | C | LYS | A | 619 | 35.299 | 53.496 | 0.336 | 1.00 | 51.91 | A | C | |
| ATOM | 570 | O | LYS | A | 619 | 35.932 | 54.396 | 0.893 | 1.00 | 53.70 | A | O | |
| ATOM | 571 | N | SER | A | 620 | 33.991 | 53.318 | 0.501 | 1.00 | 51.80 | A | N | |
| ATOM | 572 | CA | SER | A | 620 | 33.199 | 54.177 | 1.379 | 1.00 | 51.84 | A | C | |
| ATOM | 573 | CB | SER | A | 620 | 31.712 | 53.851 | 1.228 | 1.00 | 52.28 | A | C | |
| ATOM | 574 | CG | SER | A | 620 | 31.331 | 53.834 | −0.137 | 1.00 | 53.43 | A | O | |
| ATOM | 575 | C | SER | A | 620 | 33.644 | 53.875 | 2.803 | 1.00 | 52.87 | A | C | |
| ATOM | 576 | O | SER | A | 620 | 33.691 | 54.758 | 3.663 | 1.00 | 50.58 | A | O | |
| ATOM | 577 | N | THR | A | 621 | 33.981 | 52.608 | 3.024 | 1.00 | 53.81 | A | N | |
| ATOM | 578 | CA | THR | A | 621 | 34.434 | 52.111 | 4.318 | 1.00 | 60.49 | A | C | |
| ATOM | 579 | CB | THR | A | 621 | 34.817 | 50.614 | 4.220 | 1.00 | 61.16 | A | C | |
| ATOM | 580 | OG1 | THR | A | 621 | 33.704 | 49.867 | 3.714 | 1.00 | 63.62 | A | O | |
| ATOM | 581 | CG2 | THR | A | 621 | 35.206 | 50.066 | 5.587 | 1.00 | 66.57 | A | C | |
| ATOM | 582 | C | THR | A | 621 | 35.641 | 52.884 | 4.847 | 1.00 | 60.35 | A | C | |
| ATOM | 583 | O | THR | A | 621 | 35.801 | 53.049 | 6.059 | 1.00 | 59.91 | A | O | |
| ATOM | 584 | N | ALA | A | 622 | 36.484 | 53.354 | 3.931 | 1.00 | 60.73 | A | N | |
| ATOM | 585 | CA | ALA | A | 622 | 37.687 | 54.100 | 4.289 | 1.00 | 59.33 | A | C | |
| ATOM | 586 | CB | ALA | A | 622 | 38.569 | 54.282 | 3.059 | 1.00 | 51.34 | A | C | |
| ATOM | 587 | C | ALA | A | 622 | 37.380 | 55.458 | 4.913 | 1.00 | 59.82 | A | C | |
| ATOM | 588 | O | ALA | A | 622 | 36.583 | 56.235 | 4.384 | 1.00 | 58.89 | A | O | |
| ATOM | 589 | N | HIS | A | 623 | 38.026 | 55.739 | 6.040 | 1.00 | 62.90 | A | N | |
| ATOM | 590 | CA | HIS | A | 623 | 37.837 | 56.999 | 6.743 | 1.00 | 65.60 | A | C | |
| ATOM | 591 | CB | HIS | A | 623 | 37.583 | 56.734 | 8.230 | 1.00 | 69.02 | A | C | |
| ATOM | 592 | CG | HIS | A | 623 | 36.818 | 57.823 | 8.915 | 1.00 | 72.93 | A | C | |
| ATOM | 593 | CD2 | HIS | A | 623 | 35.694 | 57.783 | 9.670 | 1.00 | 75.31 | A | C | |
| ATOM | 594 | ND1 | HIS | A | 623 | 37.196 | 59.147 | 8.862 | 1.00 | 75.32 | A | N | |
| ATOM | 595 | CE1 | HIS | A | 623 | 36.338 | 59.876 | 9.554 | 1.00 | 74.30 | A | C | |
| ATOM | 596 | NE2 | HIS | A | 623 | 35.418 | 59.073 | 10.054 | 1.00 | 75.22 | A | N | |
| ATOM | 597 | C | HIS | A | 623 | 39.088 | 57.865 | 6.569 | 1.00 | 67.22 | A | C | |
| ATOM | 598 | O | HIS | A | 623 | 39.689 | 57.893 | 5.494 | 1.00 | 67.16 | A | O | |
| ATOM | 599 | N | ALA | A | 624 | 39.482 | 58.566 | 7.628 | 1.00 | 67.44 | A | N | |
| ATOM | 600 | CA | ALA | A | 624 | 40.660 | 59.426 | 7.581 | 1.00 | 66.82 | A | C | |
| ATOM | 601 | CB | ALA | A | 624 | 40.611 | 60.440 | 8.719 | 1.00 | 65.89 | A | C | |
| ATOM | 602 | C | ALA | A | 624 | 41.936 | 58.593 | 7.676 | 1.00 | 64.74 | A | C | |
| ATOM | 603 | O | ALA | A | 624 | 42.818 | 58.680 | 6.820 | 1.00 | 60.99 | A | O | |
| ATOM | 604 | N | ASP | A | 625 | 42.022 | 57.785 | 8.726 | 1.00 | 61.61 | A | N | |
| ATOM | 605 | CA | ASP | A | 625 | 43.178 | 56.929 | 8.946 | 1.00 | 65.53 | A | C | |
| ATOM | 606 | CB | ASP | A | 625 | 42.924 | 56.006 | 10.141 | 1.00 | 69.12 | A | C | |
| ATOM | 607 | CG | ASP | A | 625 | 42.556 | 56.768 | 11.401 | 1.00 | 75.92 | A | C | |
| ATOM | 608 | OD1 | ASP | A | 625 | 42.200 | 56.115 | 12.405 | 1.00 | 79.00 | A | O | |
| ATOM | 609 | OD2 | ASP | A | 625 | 42.624 | 58.017 | 11.389 | 1.00 | 77.85 | A | O | |
| ATOM | 610 | C | ASP | A | 625 | 43.458 | 56.084 | 7.707 | 1.00 | 65.81 | A | C | |
| ATOM | 611 | O | ASP | A | 625 | 44.609 | 55.777 | 7.397 | 1.00 | 64.91 | A | O | |
| ATOM | 612 | N | GLU | A | 626 | 42.393 | 55.714 | 7.003 | 1.00 | 63.78 | A | N | |
| ATOM | 613 | CA | GLU | A | 626 | 42.507 | 54.891 | 5.806 | 1.00 | 58.91 | A | C | |
| ATOM | 614 | CB | GLU | A | 626 | 41.137 | 54.314 | 5.443 | 1.00 | 65.97 | A | C | |
| ATOM | 615 | CG | GLU | A | 626 | 40.259 | 54.002 | 6.651 | 1.00 | 74.56 | A | C | |
| ATOM | 616 | CD | GLU | A | 626 | 40.898 | 53.027 | 7.619 | 1.00 | 78.91 | A | C | |
| ATOM | 617 | OE1 | GLU | A | 626 | 41.077 | 51.849 | 7.247 | 1.00 | 83.56 | A | O | |
| ATOM | 618 | OE2 | GLU | A | 626 | 41.222 | 53.440 | 8.754 | 1.00 | 80.70 | A | O | |
| ATOM | 619 | C | GLU | A | 626 | 43.055 | 55.704 | 4.636 | 1.00 | 53.87 | A | C | |
| ATOM | 620 | O | GLU | A | 626 | 43.876 | 55.212 | 3.859 | 1.00 | 44.64 | A | O | |
| ATOM | 621 | N | LYS | A | 627 | 42.595 | 56.946 | 4.510 | 1.00 | 49.35 | A | N | |
| ATOM | 622 | CA | LYS | A | 627 | 43.056 | 57.819 | 3.438 | 1.00 | 46.10 | A | C | |
| ATOM | 623 | CB | LYS | A | 627 | 42.222 | 59.102 | 3.395 | 1.00 | 52.87 | A | C | |
| ATOM | 624 | CG | LYS | A | 627 | 40.921 | 58.974 | 2.611 | 1.00 | 56.92 | A | C | |
| ATOM | 625 | CD | LYS | A | 627 | 40.205 | 60.316 | 2.509 | 1.00 | 64.69 | A | C | |
| ATOM | 626 | CE | LYS | A | 627 | 39.081 | 60.287 | 1.475 | 1.00 | 64.70 | A | C | |
| ATOM | 627 | NZ | LYS | A | 627 | 38.033 | 59.276 | 1.788 | 1.00 | 64.21 | A | N | |
| ATOM | 628 | C | LYS | A | 627 | 44.533 | 58.168 | 3.615 | 1.00 | 44.02 | A | C | |
| ATOM | 629 | O | LYS | A | 627 | 45.281 | 58.237 | 2.641 | 1.00 | 37.71 | A | O | |
| ATOM | 630 | N | GLU | A | 628 | 44.947 | 58.379 | 4.863 | 1.00 | 41.50 | A | N | |
| ATOM | 631 | CA | GLU | A | 628 | 46.335 | 58.712 | 5.164 | 1.00 | 42.46 | A | C | |
| ATOM | 632 | CB | GLU | A | 628 | 46.517 | 58.976 | 6.662 | 1.00 | 52.27 | A | C | |
| ATOM | 633 | CG | GLU | A | 628 | 45.799 | 60.211 | 7.189 | 1.00 | 60.91 | A | C | |
| ATOM | 634 | CD | GLU | A | 628 | 46.106 | 60.477 | 8.654 | 1.00 | 65.87 | A | C | |
| ATOM | 635 | OE1 | GLU | A | 628 | 47.296 | 60.665 | 8.989 | 1.00 | 68.70 | A | O | |
| ATOM | 636 | OE2 | GLU | A | 628 | 45.160 | 60.498 | 9.471 | 1.00 | 70.18 | A | O | |
| ATOM | 637 | C | GLU | A | 628 | 47.264 | 57.582 | 4.746 | 1.00 | 41.10 | A | C | |
| ATOM | 638 | O | GLU | A | 628 | 48.324 | 57.825 | 4.166 | 1.00 | 36.83 | A | O | |
| ATOM | 639 | N | ALA | A | 629 | 46.856 | 56.349 | 5.043 | 1.00 | 36.25 | A | N | |
| ATOM | 640 | CA | ALA | A | 629 | 47.646 | 55.167 | 4.715 | 1.00 | 34.22 | A | C | |
| ATOM | 641 | CB | ALA | A | 629 | 46.949 | 53.906 | 5.224 | 1.00 | 32.33 | A | C | |
| ATOM | 642 | C | ALA | A | 629 | 47.898 | 55.057 | 3.217 | 1.00 | 33.16 | A | C | |
| ATOM | 643 | O | ALA | A | 629 | 48.990 | 54.673 | 2.791 | 1.00 | 34.61 | A | O | |
| ATOM | 644 | N | LEU | A | 630 | 46.890 | 55.386 | 2.418 | 1.00 | 28.87 | A | N | |
| ATOM | 645 | CA | LEU | A | 630 | 47.040 | 55.325 | 0.968 | 1.00 | 27.43 | A | C | |
| ATOM | 646 | CE | LEU | A | 630 | 45.687 | 55.525 | 0.274 | 1.00 | 30.67 | A | C | |
| ATOM | 647 | CG | LEU | A | 630 | 45.743 | 55.479 | −1.259 | 1.00 | 27.79 | A | C | |
| ATOM | 648 | CD1 | LEU | A | 630 | 46.375 | 54.182 | −1.707 | 1.00 | 22.09 | A | C | |
| ATOM | 649 | CD2 | LEU | A | 630 | 44.349 | 55.606 | 1.838 | 1.00 | 35.18 | A | C | |
| ATOM | 650 | C | LEU | A | 630 | 48.039 | 56.392 | 0.509 | 1.00 | 24.01 | A | C | |
| ATOM | 651 | O | LEU | A | 630 | 48.923 | 56.118 | 0.309 | 1.00 | 21.58 | A | O | |
| ATOM | 652 | N | MET | A | 631 | 47.899 | 57.604 | 1.039 | 1.00 | 26.37 | A | N | |
| ATOM | 653 | CA | MET | A | 631 | 48.811 | 58.696 | 0.692 | 1.00 | 22.28 | A | C | |
| ATOM | 654 | CB | MET | A | 631 | 48.381 | 60.004 | 1.362 | 1.00 | 25.47 | A | C | |
| ATOM | 655 | CG | MET | A | 631 | 47.214 | 60.727 | 0.688 | 1.00 | 25.79 | A | C | |
| ATOM | 656 | SD | MET | A | 631 | 47.436 | 60.997 | 1.091 | 1.00 | 33.85 | A | S | |
| ATOM | 657 | CB | MET | A | 631 | 48.917 | 61.962 | 1.116 | 1.00 | 22.54 | A | C | |
| ATOM | 658 | C | MET | A | 631 | 50.234 | 58.336 | 1.126 | 1.00 | 23.32 | A | C | |
| ATOM | 659 | O | MET | A | 631 | 51.194 | 58.583 | 0.395 | 1.00 | 25.68 | A | O | |
| ATOM | 660 | N | SER | A | 632 | 50.360 | 57.762 | 2.319 | 1.00 | 20.26 | A | N | |
| ATOM | 661 | CA | SER | A | 632 | 51.660 | 57.338 | 2.837 | 1.00 | 25.00 | A | C | |
| ATOM | 662 | CB | SER | A | 632 | 51.518 | 56.764 | 4.242 | 1.00 | 22.87 | A | C | |
| ATOM | 663 | OG | SER | A | 632 | 51.210 | 57.778 | 5.172 | 1.00 | 29.83 | A | O | |
| ATOM | 664 | C | SER | A | 632 | 52.282 | 56.283 | 1.934 | 1.00 | 22.56 | A | C | |
| ATOM | 665 | O | SER | A | 632 | 53.483 | 56.320 | 1.671 | 1.00 | 20.09 | A | O | |
| ATOM | 666 | N | GLU | A | 633 | 51.469 | 55.337 | 1.463 | 1.00 | 20.83 | A | N | |
| ATOM | 667 | CA | GLU | A | 633 | 51.981 | 54.298 | 0.579 | 1.00 | 26.94 | A | C | |
| ATOM | 668 | CB | GLU | A | 633 | 50.885 | 53.301 | 0.187 | 1.00 | 25.64 | A | C | |
| ATOM | 669 | CG | GLU | A | 633 | 51.387 | 52.193 | 0.726 | 1.00 | 27.09 | A | C | |
| ATOM | 670 | CD | GLU | A | 633 | 50.280 | 51.275 | 1.201 | 1.00 | 27.05 | A | C | |
| ATOM | 671 | OE1 | GLU | A | 633 | 49.310 | 51.079 | 0.446 | 1.00 | 27.37 | A | O | |
| ATOM | 672 | OE2 | GLU | A | 633 | 50.385 | 50.738 | 2.325 | 1.00 | 33.08 | A | O | |
| ATOM | 673 | C | GLU | A | 633 | 52.493 | 54.996 | 0.666 | 1.00 | 20.38 | A | C | |
| ATOM | 674 | O | GLU | A | 633 | 53.578 | 54.698 | 1.161 | 1.00 | 21.26 | A | O | |
| ATOM | 675 | N | LEU | A | 634 | 51.701 | 55.941 | 1.156 | 1.00 | 22.56 | A | N | |
| ATOM | 676 | CA | LEU | A | 634 | 52.059 | 56.712 | 2.341 | 1.00 | 25.09 | A | C | |
| ATOM | 677 | CB | LEU | A | 634 | 50.966 | 57.741 | 2.627 | 1.00 | 27.78 | A | C | |
| ATOM | 678 | CG | LEU | A | 634 | 50.787 | 58.231 | 4.066 | 1.00 | 25.35 | A | C | |
| ATOM | 679 | CD1 | LEU | A | 634 | 49.949 | 59.512 | 4.052 | 1.00 | 21.43 | A | C | |
| ATOM | 680 | CD2 | LEU | A | 634 | 52.127 | 58.476 | 4.706 | 1.00 | 22.68 | A | C | |
| ATOM | 681 | C | LEU | A | 634 | 53.395 | 57.434 | 2.128 | 1.00 | 24.30 | A | C | |
| ATOM | 682 | O | LEU | A | 634 | 54.276 | 57.415 | 2.992 | 1.00 | 24.05 | A | O | |
| ATOM | 683 | N | LYS | A | 635 | 53.538 | 58.078 | 0.973 | 1.00 | 22.48 | A | N | |
| ATOM | 684 | CA | LYS | A | 635 | 54.764 | 58.804 | 0.646 | 1.00 | 19.32 | A | C | |
| ATOM | 685 | CB | LYS | A | 635 | 54.598 | 59.533 | 0.690 | 1.00 | 19.43 | A | C | |
| ATOM | 686 | CG | LYS | A | 635 | 53.539 | 60.612 | 0.679 | 1.00 | 27.19 | A | C | |
| ATOM | 687 | CD | LYS | A | 635 | 53.358 | 61.183 | 2.073 | 1.00 | 31.64 | A | C | |
| ATOM | 688 | CB | LYS | A | 635 | 52.221 | 62.191 | 2.122 | 1.00 | 36.37 | A | C | |
| ATOM | 689 | NZ | LYS | A | 635 | 52.063 | 62.721 | 3.502 | 1.00 | 37.87 | A | N | |
| ATOM | 690 | C | LYS | A | 635 | 55.960 | 57.857 | 0.570 | 1.00 | 15.22 | A | C | |
| ATOM | 691 | O | LYS | A | 635 | 57.061 | 58.190 | 1.018 | 1.00 | 18.34 | A | O | |
| ATOM | 692 | N | ILE | A | 636 | 55.754 | 56.680 | 0.012 | 1.00 | 18.18 | A | N | |
| ATOM | 693 | CA | ILE | A | 636 | 56.827 | 55.708 | 0.102 | 1.00 | 17.98 | A | C | |
| ATOM | 694 | CB | ILE | A | 636 | 56.403 | 54.489 | 0.941 | 1.00 | 19.18 | A | C | |
| ATOM | 695 | CG2 | ILE | A | 636 | 57.373 | 53.342 | 0.720 | 1.00 | 25.01 | A | C | |
| ATOM | 696 | CG1 | ILE | A | 636 | 56.345 | 54.880 | 2.424 | 1.00 | 20.19 | A | C | |
| ATOM | 697 | CD1 | ILE | A | 636 | 55.901 | 53.764 | 3.335 | 1.00 | 27.49 | A | C | |
| ATOM | 698 | C | ILE | A | 636 | 57.234 | 55.240 | 1.298 | 1.00 | 15.24 | A | C | |
| ATOM | 699 | O | ILE | A | 636 | 58.417 | 55.259 | −1.653 | 1.00 | 18.48 | A | O | |
| ATOM | 700 | N | MET | A | 637 | 56.255 | 54.836 | 2.098 | 1.00 | 20.21 | A | N | |
| ATOM | 701 | CA | MET | A | 637 | 56.551 | 54.352 | −3.442 | 1.00 | 17.53 | A | C | |
| ATOM | 702 | CB | MET | A | 637 | 55.291 | 53.771 | 4.086 | 1.00 | 25.94 | A | C | |
| ATOM | 703 | CG | MET | A | 637 | 54.685 | 52.585 | −3.324 | 1.00 | 21.02 | A | C | |
| ATOM | 704 | SD | MET | A | 637 | 55.855 | 51.242 | −2.971 | 1.00 | 24.05 | A | S | |
| ATOM | 705 | CB | MET | A | 637 | 56.113 | 50.547 | −4.616 | 1.00 | 18.83 | A | C | |
| ATOM | 706 | C | MET | A | 637 | 57.157 | 55.423 | −4.347 | 1.00 | 25.09 | A | C | |
| ATOM | 707 | O | MET | A | 637 | 57.901 | 55.112 | −5.277 | 1.00 | 22.66 | A | O | |
| ATOM | 708 | N | SER | A | 638 | 56.849 | 56.688 | −4.094 | 1.00 | 26.29 | A | N | |
| ATOM | 709 | CA | SER | A | 638 | 57.421 | 57.733 | −4.933 | 1.00 | 33.24 | A | C | |
| ATOM | 710 | CB | SER | A | 638 | 56.618 | 59.035 | −4.800 | 1.00 | 33.33 | A | C | |
| ATOM | 711 | OG | SER | A | 638 | 56.811 | 59.634 | −3.532 | 1.00 | 35.42 | A | O | |
| ATOM | 712 | C | SER | A | 638 | 58.877 | 57.972 | −4.530 | 1.00 | 34.32 | A | C | |
| ATOM | 713 | O | SER | A | 638 | 59.661 | 58.527 | −5.299 | 1.00 | 37.80 | A | O | |
| ATOM | 714 | N | HIS | A | 639 | 59.231 | 57.519 | −3.328 | 1.00 | 30.10 | A | N | |
| ATOM | 715 | CA | HIS | A | 639 | 60.573 | 57.694 | −2.781 | 1.00 | 34.75 | A | C | |
| ATOM | 716 | CB | HIS | A | 639 | 60.451 | 58.134 | −1.316 | 1.00 | 40.02 | A | C | |
| ATOM | 717 | CG | HIS | A | 639 | 61.721 | 58.026 | −0.531 | 1.00 | 45.54 | A | C | |
| ATOM | 718 | CD2 | HIS | A | 639 | 62.589 | 58.974 | −0.104 | 1.00 | 50.09 | A | C | |
| ATOM | 719 | ND1 | HIS | A | 639 | 62.207 | 56.823 | −0.061 | 1.00 | 52.72 | A | N | |
| ATOM | 720 | CE1 | HIS | A | 639 | 63.317 | 57.036 | 0.623 | 1.00 | 54.80 | A | C | |
| ATOM | 721 | NE2 | HIS | A | 639 | 63.571 | 58.333 | 0.612 | 1.00 | 54.49 | A | N | |
| ATOM | 722 | C | HIS | A | 639 | 61.510 | 56.484 | −2.897 | 1.00 | 32.89 | A | C | |
| ATOM | 723 | O | HIS | A | 639 | 62.732 | 56.638 | −2.874 | 1.00 | 23.77 | A | O | |
| ATOM | 724 | N | LEU | A | 640 | 60.948 | 55.288 | −3.035 | 1.00 | 26.57 | A | N | |
| ATOM | 725 | CA | LEU | A | 640 | 61.767 | 54.088 | −3.138 | 1.00 | 26.21 | A | C | |
| ATOM | 726 | CB | LEU | A | 640 | 60.901 | 52.833 | −2.988 | 1.00 | 24.80 | A | C | |
| ATOM | 727 | CG | LEU | A | 640 | 60.546 | 52.323 | −1.591 | 1.00 | 28.55 | A | C | |
| ATOM | 728 | CD1 | LEU | A | 640 | 59.736 | 51.039 | −1.739 | 1.00 | 31.89 | A | C | |
| ATOM | 729 | CD2 | LEU | A | 640 | 61.803 | 52.054 | −0.785 | 1.00 | 32.23 | A | C | |
| ATOM | 730 | C | LEU | A | 640 | 62.581 | 53.943 | −4.424 | 1.00 | 24.89 | A | C | |
| ATOM | 731 | O | LEU | A | 640 | 63.653 | 53.349 | −4.415 | 1.00 | 22.91 | A | O | |
| ATOM | 732 | N | GLY | A | 641 | 62.083 | 54.481 | −5.528 | 1.00 | 24.86 | A | N | |
| ATOM | 733 | CA | GLY | A | 641 | 62.804 | 54.309 | −6.773 | 1.00 | 28.59 | A | C | |
| ATOM | 734 | C | GLY | A | 641 | 62.340 | 52.984 | −7.354 | 1.00 | 29.97 | A | C | |
| ATOM | 735 | O | GLY | A | 641 | 61.725 | 52.179 | −6.647 | 1.00 | 27.41 | A | O | |
| ATOM | 736 | N | GLN | A | 642 | 62.637 | 52.731 | −8.624 | 1.00 | 24.00 | A | N | |
| ATOM | 737 | CA | GLN | A | 642 | 62.189 | 51.497 | −9.261 | 1.00 | 27.01 | A | C | |
| ATOM | 738 | CB | GLN | A | 642 | 61.919 | 51.728 | −10.751 | 1.00 | 29.07 | A | C | |
| ATOM | 739 | CG | GLN | A | 642 | 60.775 | 52.683 | −11.054 | 1.00 | 52.49 | A | C | |
| ATOM | 740 | CD | GLN | A | 642 | 60;420 | 52.701 | −12.532 | 1.00 | 62.87 | A | C | |
| ATOM | 741 | OE1 | GLN | A | 642 | 61.287 | 52.903 | −13.386 | 1.00 | 68.48 | A | O | |
| ATOM | 742 | NE2 | GLN | A | 642 | 59.143 | 52.490 | −12.840 | 1.00 | 63.12 | A | N | |
| ATOM | 743 | C | GLN | A | 642 | 63.133 | 50.311 | −9.136 | 1.00 | 22.61 | A | C | |
| ATOM | 744 | O | GLN | A | 642 | 64.340 | 50.474 | −8.951 | 1.00 | 21.65 | A | O | |
| ATOM | 745 | N | HIS | A | 643 | 62.556 | 49.118 | −9.251 | 1.00 | 22.32 | A | N | |
| ATOM | 746 | CA | HIS | A | 643 | 63.302 | 47.868 | −9.205 | 1.00 | 22.22 | A | C | |
| ATOM | 747 | CB | HIS | A | 643 | 63.536 | 47.410 | −7.756 | 1.00 | 21.85 | A | C | |
| ATOM | 748 | CG | HIS | A | 643 | 64.434 | 46.211 | −7.634 | 1.00 | 23.29 | A | C | |
| ATOM | 749 | CD2 | HIS | A | 643 | 65.738 | 46.107 | −7.278 | 1.00 | 25.57 | A | C | |
| ATOM | 750 | ND1 | HIS | A | 643 | 64.012 | 44.927 | −7.912 | 1.00 | 23.34 | A | N | |
| ATOM | 751 | CE1 | HIS | A | 643 | 65.016 | 44.085 | −7.730 | 1.00 | 20.93 | A | C | |
| ATOM | 752 | NE2 | HIS | A | 643 | 66.075 | 44.776 | −7.344 | 1.00 | 24.44 | A | N | |
| ATOM | 753 | C | HIS | A | 643 | 62.499 | 46.825 | −9.987 | 1.00 | 23.97 | A | C | |
| ATOM | 754 | O | HIS | A | 643 | 61.261 | 46.825 | −9.988 | 1.00 | 20.55 | A | O | |
| ATOM | 755 | N | GLU | A | 644 | 63.213 | 45.936 | −10.662 | 1.00 | 28.13 | A | N | |
| ATOM | 756 | CA | GLU | A | 644 | 62.587 | 44.903 | −11.476 | 1.00 | 29.69 | A | C | |
| ATOM | 757 | CB | GLU | A | 644 | 63.684 | 44.094 | −12.181 | 1.00 | 38.10 | A | C | |
| ATOM | 758 | CG | GLU | A | 644 | 64.613 | 43.353 | −11.222 | 1.00 | 52.65 | A | C | |
| ATOM | 759 | CD | GLU | A | 644 | 65.828 | 42.747 | −11.909 | 1.00 | 61.25 | A | C | |
| ATOM | 760 | OE1 | GLU | A | 644 | 65.656 | 42.080 | −12.953 | 1.00 | 63.57 | A | O | |
| ATOM | 761 | OE2 | GLU | A | 644 | 66.955 | 42.929 | −11.396 | 1.00 | 64.44 | A | O | |
| ATOM | 762 | C | GLU | A | 644 | 61.642 | 43.948 | −10.729 | 1.00 | 27.63 | A | C | |
| ATOM | 763 | O | GLU | A | 644 | 60.710 | 43.401 | −11.327 | 1.00 | 21.30 | A | O | |
| ATOM | 764 | N | ASN | A | 645 | 61.861 | 43.752 | −9.429 | 1.00 | 22.24 | A | N | |
| ATOM | 765 | CA | ASN | A | 645 | 61.021 | 42.821 | −8.674 | 1.00 | 25.56 | A | C | |
| ATOM | 766 | CB | ASN | A | 645 | 61.901 | 41.809 | −7.939 | 1.00 | 19.60 | A | C | |
| ATOM | 767 | CC | ASN | A | 645 | 62.854 | 41.097 | −8.877 | 1.00 | 27.53 | A | C | |
| ATOM | 768 | OD1 | ASN | A | 645 | 64.077 | 41.220 | −8.756 | 1.00 | 21.54 | A | O | |
| ATOM | 769 | ND2 | ASN | A | 645 | 62.296 | 40.360 | −9.835 | 1.00 | 17.26 | A | N | |
| ATOM | 770 | C | ASN | A | 645 | 60.050 | 43.469 | −7.701 | 1.00 | 26.56 | A | C | |
| ATOM | 771 | O | ASN | A | 645 | 59.604 | 42.844 | −6.746 | 1.00 | 22.63 | A | O | |
| ATOM | 772 | N | ILE | A | 646 | 59.737 | 44.732 | −7.949 | 1.00 | 24.07 | A | N | |
| ATOM | 773 | CA | ILE | A | 646 | 58.792 | 45.463 | −7.127 | 1.00 | 22.29 | A | C | |
| ATOM | 774 | CB | ILE | A | 646 | 59.430 | 46.727 | −6.508 | 1.00 | 23.88 | A | C | |
| ATOM | 775 | CG2 | ILE | A | 646 | 58.393 | 47.491 | −5.705 | 1.00 | 30.07 | A | C | |
| ATOM | 776 | CG1 | ILE | A | 646 | 60.617 | 46.353 | −5.621 | 1.00 | 28.07 | A | C | |
| ATOM | 777 | CD1 | ILE | A | 646 | 60.240 | 45.844 | −4.249 | 1.00 | 26.14 | A | C | |
| ATOM | 778 | C | ILE | A | 646 | 57.677 | 45.905 | −8.069 | 1.00 | 23.35 | A | C | |
| ATOM | 779 | O | ILE | A | 646 | 57.929 | 46.179 | −9.251 | 1.00 | 22.79 | A | O | |
| ATOM | 780 | N | VAL | A | 647 | 56.447 | 45.933 | −7.566 | 1.00 | 18.17 | A | N | |
| ATOM | 781 | CA | VAL | A | 647 | 55.311 | 46.401 | −8.357 | 1.00 | 23.59 | A | C | |
| ATOM | 782 | CB | VAL | A | 647 | 53.970 | 46.004 | −7.706 | 1.00 | 17.76 | A | C | |
| ATOM | 783 | CG1 | VAL | A | 647 | 52.816 | 46.598 | −8.486 | 1.00 | 20.21 | A | C | |
| ATOM | 784 | CG2 | VAL | A | 647 | 53.846 | 44.484 | −7.666 | 1.00 | 27.63 | A | C | |
| ATOM | 785 | C | VAL | A | 647 | 55.486 | 47.923 | −8.318 | 1.00 | 26.28 | A | C | |
| ATOM | 786 | O | VAL | A | 647 | 55.023 | 48.604 | −7.396 | 1.00 | 23.39 | A | O | |
| ATOM | 787 | N | ASN | A | 648 | 56.175 | 48.442 | −9.326 | 1.00 | 25.50 | A | N | |
| ATOM | 788 | CA | ASN | A | 648 | 56.492 | 49.859 | −9.393 | 1.00 | 24.16 | A | C | |
| ATOM | 789 | CB | ASN | A | 648 | 57.496 | 50.100 | −10.526 | 1.00 | 26.76 | A | C | |
| ATOM | 790 | CG | ASN | A | 648 | 58.806 | 49.358 | −10.310 | 1.00 | 29.70 | A | C | |
| ATOM | 791 | OD1 | ASN | A | 648 | 59.478 | 49.537 | −9.292 | 1.00 | 23.12 | A | O | |
| ATOM | 792 | ND2 | ASN | A | 648 | 59.174 | 48.520 | −11.267 | 1.00 | 28.39 | A | N | |
| ATOM | 793 | C | ASN | A | 648 | 55.333 | 50.838 | −9.519 | 1.00 | 21.87 | A | C | |
| ATOM | 794 | O | ASN | A | 648 | 54.292 | 50.530 | −10.091 | 1.00 | 23.06 | A | O | |
| ATOM | 795 | N | LEU | A | 649 | 55.528 | 52.020 | −8.945 | 1.00 | 21.70 | A | N | |
| ATOM | 796 | CA | LEU | A | 649 | 54.540 | 53.080 | −9.021 | 1.00 | 20.39 | A | C | |
| ATOM | 797 | CB | LEU | A | 649 | 54.832 | 54.162 | −7.978 | 1.00 | 20.12 | A | C | |
| ATOM | 798 | CG | LEU | A | 649 | 53.994 | 55.438 | −8.100 | 1.00 | 22.61 | A | C | |
| ATOM | 799 | CD1 | LEU | A | 649 | 52.541 | 55.132 | −7.779 | 1.00 | 17.80 | A | C | |
| ATOM | 800 | CD2 | LEU | A | 649 | 54.539 | 56.505 | −7.148 | 1.00 | 25.20 | A | C | |
| ATOM | 801 | C | LEU | A | 649 | 54.705 | 53.664 | −10.421 | 1.00 | 22.86 | A | C | |
| ATOM | 802 | O | LEU | A | 649 | 55.826 | 53.803 | −10.916 | 1.00 | 23.25 | A | O | |
| ATOM | 803 | N | LEU | A | 650 | 53.592 | 53.997 | −11.058 | 1.00 | 21.92 | A | N | |
| ATOM | 804 | CA | LEU | A | 650 | 53.624 | 54.561 | −12.396 | 1.00 | 24.37 | A | C | |
| ATOM | 805 | CB | LEU | A | 650 | 52.854 | 53.647 | −13.357 | 1.00 | 24.72 | A | C | |
| ATOM | 806 | CG | LEU | A | 650 | 53.343 | 52.196 | −13.493 | 1.00 | 25.79 | A | C | |
| ATOM | 807 | CD1 | LEU | A | 650 | 52.409 | 51.427 | −14.418 | 1.00 | 31.72 | A | C | |
| ATOM | 808 | CD2 | LEU | A | 650 | 54.758 | 52.170 | −14.038 | 1.00 | 24.15 | A | C | |
| ATOM | 809 | C | LEU | A | 650 | 53.016 | 55.965 | −12.404 | 1.00 | 25.19 | A | C | |
| ATOM | 810 | O | LEU | A | 650 | 53.414 | 56.819 | −13.192 | 1.00 | 29.30 | A | O | |
| ATOM | 811 | N | GLY | A | 651 | 52.048 | 56.194 | −11.524 | 1.00 | 25.81 | A | N | |
| ATOM | 812 | CA | GLY | A | 651 | 51.410 | 57.494 | −11.450 | 1.00 | 27.92 | A | C | |
| ATOM | 813 | C | GLY | A | 651 | 50.340 | 57.522 | −10.378 | 1.00 | 29.85 | A | C | |
| ATOM | 814 | O | GLY | A | 651 | 50.115 | 56.528 | −9.688 | 1.00 | 27.19 | A | O | |
| ATOM | 815 | N | ALA | A | 652 | 49.681 | 58.667 | −10.241 | 1.00 | 25.92 | A | N | |
| ATOM | 816 | CA | ALA | A | 652 | 48.623 | 58.840 | −9.265 | 1.00 | 20.70 | A | C | |
| ATOM | 817 | CB | ALA | A | 652 | −49.218 | 59.231 | −7.914 | 1.00 | 15.45 | A | C | |
| ATOM | 818 | C | ALA | A | 652 | 47.657 | 59.923 | −9.738 | 1.00 | 27.72 | A | C | |
| ATOM | 819 | O | ALA | A | 652 | 47.982 | 60.726 | −10.613 | 1.00 | 28.78 | A | O | |
| ATOM | 820 | N | CYS | A | 653 | 46.464 | 59.926 | −9.157 | 1.00 | 27.84 | A | N | |
| ATOM | 821 | CA | CYS | A | 653 | 45.436 | 60.910 | −9.466 | 1.00 | 27.49 | A | C | |
| ATOM | 822 | CB | CYS | A | 653 | 44.285 | 60.254 | −10.230 | 1.00 | 26.09 | A | C | |
| ATOM | 823 | SG | CYS | A | 653 | 44.784 | 59.486 | −11.802 | 1.00 | 34.38 | A | S | |
| ATOM | 824 | C | CYS | A | 653 | 44.980 | 61.379 | −8.088 | 1.00 | 26.02 | A | C | |
| ATOM | 825 | O | CYS | A | 653 | 44.175 | 60.728 | −7.437 | 1.00 | 27.77 | A | O | |
| ATOM | 826 | N | THR | A | 654 | 45.517 | 62.507 | −7.640 | 1.00 | 24.22 | A | N | |
| ATOM | 827 | CA | THR | A | 654 | 45.190 | 63.020 | −6.321 | 1.00 | 26.99 | A | C | |
| ATOM | 828 | CB | THR | A | 654 | 46.477 | 63.311 | −5.515 | −1.00 | 29.57 | A | C | |
| ATOM | 829 | OG1 | THR | A | 654 | 47.257 | 64.304 | −6.193 | 1.00 | 34.03 | A | O | |
| ATOM | 830 | CG2 | THR | A | 654 | 47.309 | 62.047 | −5.368 | 1.00 | 28.82 | A | C | |
| ATOM | 831 | C | THR | A | 654 | 44.33 | 264.280 | −6.350 | 1.00 | 29.01 | A | C | |
| ATOM | 832 | O | THR | A | 654 | 44.098 | 64.896 | −5.312 | 1.00 | 31.97 | A | O | |
| ATOM | 833 | N | HIS | A | 655 | 43.862 | 64.654 | −7.536 | 1.00 | 31.62 | A | N | |
| ATOM | 834 | CA | HIS | A | 655 | 43.029 | 65.844 | −7.687 | 1.00 | 34.93 | A | C | |
| ATOM | 835 | CB | HIS | A | 655 | 43.838 | 66.981 | −8.323 | 1.00 | 31.60 | A | C | |
| ATOM | 836 | CG | HIS | A | 655 | 45.071 | 67.345 | −7.559 | 1.00 | 37.08 | A | C | |
| ATOM | 837 | CD2 | HIS | A | 655 | 46.382 | 67.207 | −7.865 | 1.00 | 30.91 | A | C | |
| ATOM | 838 | ND1 | HIS | A | 655 | 45.029 | 67.894 | −6.295 | 1.00 | 37.45 | A | N | |
| ATOM | 839 | CE1 | HIS | A | 655 | 46.261 | 68.076 | −5.855 | 1.00 | 37.22 | A | C | |
| ATOM | 840 | NE2 | HIS | A | 655 | 47.101 | 67.666 | −6.789 | 1.00 | 33.14 | A | N | |
| ATOM | 841 | C | HIS | A | 655 | 41.807 | 65.562 | −8.551 | 1.00 | 36.19 | A | C | |
| ATOM | 842 | O | HIS | A | 655 | 41.804 | 64.629 | −9.359 | 1.00 | 37.26 | A | O | |
| ATOM | 843 | N | GLY | A | 656 | 40.772 | 66.381 | −8.372 | 1.00 | 36.44 | A | N | |
| ATOM | 844 | CA | GLY | A | 656 | 39.554 | 66.239 | −9.148 | 1.00 | 33.74 | A | C | |
| ATOM | 845 | C | GLY | A | 656 | 38.774 | 64.961 | −8.9.14 | 1.00 | 32.51 | A | C | |
| ATOM | 846 | O | GLY | A | 656 | 37.958 | 64.566 | −9.747 | 1.00 | 35.25 | A | O | |
| ATOM | 847 | N | GLY | A | 657 | 39.015 | 64.317 | −7.779 | 1.00 | 31.89 | A | N | |
| ATOM | 848 | CA | GLY | A | 657 | 38.319 | 63.083 | −7.467 | 1.00 | 34.11 | A | C | |
| ATOM | 849 | C | GLY | A | 657 | 39.053 | 62.290 | −6.403 | 1.00 | 34.68 | A | C | |
| ATOM | 850 | O | GLY | A | 657 | 40.048 | 62.762 | −5.856 | 1.00 | 31.99 | A | O | |
| ATOM | 851 | N | PRO | A | 658 | 38.579 | 61.080 | −6.079 | 1.00 | 39.26 | A | N | |
| ATOM | 852 | CD | PRO | A | 658 | 37.341 | 60.450 | −6.567 | 1.00 | 43.66 | A | C | |
| ATOM | 853 | CA | PRO | A | 658 | 39.224 | 60.242 | −5.062 | 1.00 | 37.22 | A | C | |
| ATOM | 854 | CB | PRO | A | 658 | 38.302 | 59.024 | −4.981 | 1.00 | 39.65 | A | C | |
| ATOM | 855 | CG | PRO | A | 658 | 36.963 | 59.575 | −5.405 | 1.00 | 43.48 | A | C | |
| ATOM | 856 | C | PRO | A | 658 | 40.635 | 59.861 | −5.500 | 1.00 | 33.73 | A | C | |
| ATOM | 857 | O | PRO | A | 658 | 40.907 | 59.715 | −6.695 | 1.00 | 28.06 | A | O | |
| ATOM | 858 | N | VAL | A | 659 | 41.525 | 59.697 | −4.528 | 1.00 | 31.88 | A | N | |
| ATOM | 859 | CA | VAL | A | 659 | 42.909 | 59.329 | −4.804 | 1.00 | 31.94 | A | C | |
| ATOM | 860 | CB | VAL | A | 659 | 43.736 | 59.269 | −3.499 | 1.00 | 34.29 | A | C | |
| ATOM | 861 | CG1 | VAL | A | 659 | 45.165 | 58.823 | −3.809 | 1.00 | 29.89 | A | C | |
| ATOM | 862 | CG2 | VAL | A | 659 | 43.734 | 60.626 | −2.818 | 1.00 | 32.93 | A | C | |
| ATOM | 863 | C | VAL | A | 659 | 43.035 | 57.975 | −5.501 | 1.00 | 24.77 | A | C | |
| ATOM | 864 | O | VAL | A | 659 | 42.442 | 56.991 | −5.070 | 1.00 | 30.57 | A | O | |
| ATOM | 865 | N | LEU | A | 660 | 43.813 | 57.932 | −6.578 | 1.00 | 25.76 | A | N | |
| ATOM | 866 | CA | LEU | A | 660 | 44.044 | 56.694 | −7.321 | 1.00 | 26.43 | A | C | |
| ATOM | 867 | CB | LEU | A | 660 | 43.426 | 56.772 | −8.722 | 1.00 | 29.65 | A | C | |
| ATOM | 868 | CG | LEU | A | 660 | 41.965 | 57.227 | −8.845 | 1.00 | 35.40 | A | C | |
| ATOM | 869 | CD1 | LEU | A | 660 | 41.561 | 57.235 | −10.318 | 1.00 | 31.66 | A | C | |
| ATOM | 870 | CD2 | LEU | A | 660 | 41.053 | 56.316 | −8.041 | 1.00 | 27.07 | A | C | |
| ATOM | 871 | C | LEU | A | 660 | 45.552 | 56.496 | −7.457 | 1.00 | 26.44 | A | C | |
| ATOM | 872 | O | LEU | A | 660 | 46.260 | 57.395 | −7.910 | 1.00 | 27.80 | A | O | |
| ATOM | 873 | N | VAL | A | 661 | 46.042 | 55.332 | −7.050 | 1.00 | 22.60 | A | N | |
| ATOM | 874 | CA | VAL | A | 661 | 47.464 | 55.023 | −7.151 | 1.00 | 23.64 | A | C | |
| ATOM | 875 | CB | VAL | A | 661 | 47.998 | 54.463 | −5.807 | 1.00 | 6.14 | A | C | |
| ATOM | 876 | CG1 | VAL | A | 661 | 49.419 | 53.931 | −5.976 | 1.00 | 23.41 | A | C | |
| ATOM | 877 | CG2 | VAL | A | 661 | 47.946 | 55.563 | −4.744 | 1.00 | 20.58 | A | C | |
| ATOM | 878 | C | VAL | A | 661 | 47.643 | 54.000 | −8.273 | 1.00 | 23.24 | A | C | |
| ATOM | 879 | O | VAL | A | 661 | 47.134 | 52.882 | −6.206 | 1.00 | 21.85 | A | O | |
| ATOM | 880 | N | ILE | A | 662 | 48.361 | 54.405 | −9.312 | 1.00 | 25.10 | A | N | |
| ATOM | 881 | CA | ILE | A | 662 | 48.595 | 53.561 | −10.471 | 1.00 | 26.03 | A | C | |
| ATOM | 882 | CB | ILE | A | 662 | 48.554 | 54.399 | −11.765 | 1.00 | 26.14 | A | C | |
| ATOM | 883 | CG2 | ILE | A | 662 | 48.543 | 53.475 | −12.982 | 1.00 | 28.43 | A | C | |
| ATOM | 884 | CG1 | ILE | A | 662 | 47.311 | 55.294 | −11.765 | 1.00 | 30.10 | A | C | |
| ATOM | 885 | CD1 | ILE | A | 662 | 47.344 | 56.393 | −12.824 | 1.00 | 23.69 | A | C | |
| ATOM | 886 | C | ILE | A | 662 | 49.954 | 52.873 | −10.366 | 1.00 | 27.61 | A | C | |
| ATOM | 887 | O | ILE | A | 662 | 50.968 | 53.531 | −10.260 | 1.01 | 29.04 | A | O | |
| ATOM | 888 | N | THR | A | 663 | 49.942 | 51.547 | −10.462 | 1.00 | 29.00 | A | N | |
| ATOM | 889 | CA | THR | A | 663 | 51.166 | 50.755 | −10.395 | 1.00 | 28.88 | A | C | |
| ATOM | 890 | CB | THR | A | 663 | 51.238 | 49.955 | −9.078 | 1.00 | 30.13 | A | C | |
| ATOM | 891 | OG1 | THR | A | 663 | 50.304 | 48.873 | −9.129 | 1.00 | 25.31 | A | O | |
| ATOM | 892 | CG2 | THR | A | 663 | 50.878 | 50.841 | −7.898 | 1.00 | 28.52 | A | C | |
| ATOM | 893 | C | THR | A | 663 | 51.200 | 49.755 | −11.549 | 1.00 | 30.38 | A | C | |
| ATOM | 894 | O | THR | A | 663 | 50.227 | 49.612 | −12.293 | 1.00 | 31.59 | A | O | |
| ATOM | 895 | N | GLU | A | 664 | 52.327 | 49.070 | −11.696 | 1.00 | 27.00 | A | N | |
| ATOM | 896 | CA | GLU | A | 664 | 52.466 | 48.068 | −12.743 | 1.00 | 35.00 | A | C | |
| ATOM | 897 | CB | GLU | A | 664 | 53.875 | 47.475 | −12.740 | 1.00 | 29.30 | A | C | |
| ATOM | 898 | CG | GLU | A | 664 | 54.965 | 48.469 | −13.083 | 1.00 | 37.47 | A | C | |
| ATOM | 899 | CD | GLU | A | 664 | 56.349 | 47.846 | −13.052 | 1.00 | 41.46 | A | C | |
| ATOM | 900 | OE1 | GLU | A | 664 | 56.715 | 47.254 | −12.017 | 1.00 | 35.89 | A | O | |
| ATOM | 901 | OE2 | GLU | A | 664 | 57.074 | 47.950 | −14.064 | 1.00 | 48.46 | A | O | |
| ATOM | 902 | C | GLU | A | 664 | 51.445 | 46.957 | −12.519 | 1.00 | 28.73 | A | C | |
| ATOM | 903 | O | GLU | A | 664 | 51.031 | 46.692 | −11.391 | 1.00 | 30.61 | A | O | |
| ATOM | 904 | N | TYR | A | 665 | 51.044 | 46.310 | −13.604 | 1.00 | 30.70 | A | N | |
| ATOM | 905 | CA | TYR | A | 665 | 50.073 | 45.228 | −13.547 | 1.00 | 29.76 | A | C | |
| ATOM | 906 | CB | TYR | A | 665 | 49.030 | 45.438 | −14.652 | 1.00 | 32.72 | A | C | |
| ATOM | 907 | CG | TYR | A | 665 | 48.144 | 44.248 | −14.922 | 1.00 | 35.89 | A | C | |
| ATOM | 908 | CD1 | TYR | A | 665 | 47.224 | 43.810 | −13.978 | 1.00 | 27.69 | A | C | |
| ATOM | 909 | CE1 | TYR | A | 665 | 46.423 | 42.713 | −14.224 | 1.00 | 34.20 | A | C | |
| ATOM | 910 | CD2 | TYR | A | 665 | 48.237 | 43.556 | −16.121 | 1.00 | 31.55 | A | C | |
| ATOM | 911 | CE2 | TYR | A | 665 | 47.441 | 42.459 | −16.375 | 1.00 | 31.36 | A | C | |
| ATOM | 912 | CZ | TYR | A | 665 | 46.538 | 42.042 | −15.425 | 1.00 | 28.75 | A | C | |
| ATOM | 913 | OH | TYR | A | 665 | 45.753 | 40.944 | −15.675 | 1.00 | 35.96 | A | O | |
| ATOM | 914 | C | TYR | A | 665 | 50.779 | 43.878 | −13.716 | 1.00 | 28.14 | A | C | |
| ATOM | 915 | O | TYR | A | 665 | 51.714 | 43.750 | −14.508 | 1.00 | 30.04 | A | O | |
| ATOM | 916 | N | CYS | A | 666 | 50.337 | 42.875 | −12.964 | 1.00 | 29.53 | A | N | |
| ATOM | 917 | CA | CYS | A | 666 | 50.941 | 41.545 | −13.036 | 1.00 | 28.64 | A | C | |
| ATOM | 918 | CB | CYS | A | 666 | 51.411 | 41.124 | −11.638 | 1.00 | 25.70 | A | C | |
| ATOM | 919 | SG | CYS | A | 666 | 52.670 | 42.237 | −10.910 | 1.00 | 26.77 | A | S | |
| ATOM | 920 | C | CYS | A | 666 | 49.951 | 40.522 | −13.625 | 1.00 | 29.12 | A | C | |
| ATOM | 921 | O | CYS | A | 666 | 49.122 | 39.952 | −12.916 | 1.00 | 22.94 | A | O | |
| ATOM | 922 | N | CYS | A | 667 | 50.068 | 40.295 | −14.932 | 1.00 | 30.40 | A | N | |
| ATOM | 923 | CA | CYS | A | 667 | 49.181 | 39.396 | −15.672 | 1.00 | 34.47 | A | C | |
| ATOM | 924 | CB | CYS | A | 667 | 49.639 | 39.308 | −17.135 | 1.00 | 36.28 | A | C | |
| ATOM | 925 | SG | CYS | A | 667 | 51.310 | 38.651 | −17.411 | 1.00 | 40.31 | A | S | |
| ATOM | 926 | C | CYS | A | 667 | 48.924 | 37.978 | −15.164 | 1.00 | 35.66 | A | C | |
| ATOM | 927 | O | CYS | A | 667 | 47.874 | 37.413 | −15.459 | 1.00 | 38.24 | A | O | |
| ATOM | 928 | N | TYR | A | 668 | 49.849 | 37.397 | −14.405 | 1.00 | 36.54 | A | N | |
| ATOM | 929 | CA | TYR | A | 668 | 49.653 | 36.025 | −13.934 | 1.00 | 32.77 | A | C | |
| ATOM | 930 | CB | TYR | A | 668 | 50.959 | 35.238 | −14.074 | 1.00 | 37.24 | A | C | |
| ATOM | 931 | CG | TYR | A | 668 | 51.417 | 35.083 | −15.511 | 1.00 | 33.30 | A | C | |
| ATOM | 932 | CD1 | TYR | A | 668 | 52.665 | 35.536 | −15.915 | 1.00 | 33.99 | A | C | |
| ATOM | 933 | CE1 | TYR | A | 668 | 53.073 | 35.435 | −17.229 | 1.00 | 39.96 | A | C | |
| ATOM | 934 | CD2 | TYR | A | 668 | 50.585 | 34.515 | −16.469 | 1.00 | 38.12 | A | C | |
| ATOM | 935 | CE2 | TYR | A | 668 | 50.985 | 34.408 | −17.790 | 1.00 | 34.76 | A | C | |
| ATOM | 936 | CZ | TYR | A | 668 | 52.229 | 34.872 | −18.165 | 1.00 | 39.12 | A | C | |
| ATOM | 937 | OH | TYR | A | 668 | 52.629 | 34.790 | −19.481 | 1.00 | 41.65 | A | O | |
| ATOM | 938 | C | TYR | A | 668 | 49.088 | 35.840 | −12.528 | 1.00 | 34.16 | A | C | |
| ATOM | 939 | O | TYR | A | 668 | 48.866 | 34.709 | −12.097 | 1.00 | 33.71 | A | O | |
| ATOM | 940 | N | GLY | A | 669 | 48.853 | 36.932 | −11.809 | 1.00 | 26.49 | A | N | |
| ATOM | 941 | CA | GLY | A | 669 | 48.297 | 36.805 | −10.470 | 1.00 | 28.48 | A | C | |
| ATOM | 942 | C | GLY | A | 669 | 49.300 | 36.486 | −9.375 | 1.00 | 23.20 | A | C | |
| ATOM | 943 | O | GLY | A | 669 | 50.515 | 36.592 | −9.578 | 1.00 | 23.60 | A | O | |
| ATOM | 944 | N | ASP | A | 670 | 48.800 | 36.084 | −8.210 | 1.00 | 24.97 | A | N | |
| ATOM | 945 | CA | ASP | A | 670 | 49.685 | 35.787 | −7.095 | 1.00 | 28.97 | A | C | |
| ATOM | 946 | CB | ASP | A | 670 | 48.971 | 35.992 | −5.749 | 1.00 | 27.18 | A | C | |
| ATOM | 947 | CC | ASP | A | 670 | 48.065 | 34.848 | −5.401 | 1.00 | 37.51 | A | C | |
| ATOM | 948 | OD1 | ASP | A | 670 | 46.987 | 34.745 | −6.017 | 1.00 | 42.45 | A | O | |
| ATOM | 949 | OD2 | ASP | A | 670 | 48.438 | 34.045 | −4.520 | 1.00 | 37.32 | A | O | |
| ATOM | 950 | C | ASP | A | 670 | 50.309 | 34.398 | −7.140 | 1.00 | 27.49 | A | C | |
| ATOM | 951 | O | ASP | A | 670 | 49.713 | 33.415 | −7.610 | 1.00 | 25.73 | A | O | |
| ATOM | 952 | N | LEU | A | 671 | 51.535 | 34.352 | −6.644 | 1.00 | 26.96 | A | N | |
| ATOM | 953 | CA | LEU | A | 671 | 52.346 | 33.149 | −6.591 | 1.00 | 22.40 | A | C | |
| ATOM | 954 | CB | LEU | A | 671 | 53.678 | 33.478 | −5.922 | 1.00 | 21.85 | A | C | |
| ATOM | 955 | CG | LEU | A | 671 | 54.595 | 32.293 | −5.630 | 1.00 | 17.71 | A | C | |
| ATOM | 956 | CD1 | LEU | A | 671 | 55.022 | 31.646 | 6.933 | 1.00 | 21.96 | A | C | |
| ATOM | 957 | CD2 | LEU | A | 671 | 55.797 | 32.782 | −4.852 | 1.00 | 20.20 | A | C | |
| ATOM | 958 | C | LEU | A | 671 | 51.713 | 31.960 | −5.883 | 1.00 | 27.28 | A | C | |
| ATOM | 959 | O | LEU | A | 671 | 51.769 | 30.842 | −6.388 | 1.00 | 23.81 | A | O | |
| ATOM | 960 | N | LEU | A | 672 | 51.120 | 32.188 | −4.712 | 1.00 | 21:.21 | A | N | |
| ATOM | 961 | CA | LEU | A | 672 | 50.519 | 31.090 | −3.968 | 1.00 | 21.54 | A | C | |
| ATOM | 962 | CB | LEU | A | 672 | 49.776 | 31.605 | −2.737 | 1.00 | 23.59 | A | C | |
| ATOM | 963 | CG | LEU | A | 672 | 49.217 | 30.455 | −1.894 | .1.00 | 24.90 | A | C | |
| ATOM | 964 | CD1 | LEU | A | 672 | 50.355 | 29.528 | −1.508 | 1.00 | 2930 | A | C | |
| ATOM | 965 | CD2 | LEU | A | 672 | 48.511 | 30.983 | −0.671 | 1.00 | 22.25 | A | C | |
| ATOM | 966 | C | LEU | A | 672 | 49.562 | 30.243 | −4.805 | 1.00 | 29.90 | A | C | |
| ATOM | 967 | O | LEU | A | 672 | 49.611 | 29.010 | −4.767 | 1.00 | 24.61 | A | O | |
| ATOM | 968 | N | ASN | A | 673 | 48.693 | 30.895 | 15.564 | 1.00 | 25.27 | A | N | |
| ATOM | 969 | CA | ASN | A | 673 | 47.749 | 30.151 | −6.383 | 1.00 | 28.81 | A | C | |
| ATOM | 970 | CB | ASN | A | 673 | 46.616 | 31.071 | −6.827 | 1.00 | 35.55 | A | C | |
| ATOM | 971 | CG | ASN | A | 673 | 45.717 | 31.455 | −5.670 | 1.00 | 43.59 | A | C | |
| ATOM | 972 | OD1 | ASN | A | 673 | 45.093 | 30.597 | −5.046 | 1.00 | 48.83 | A | O | |
| ATOM | 973 | ND2 | ASN | A | 673 | 45.662 | 32.741 | −5.362 | 1.00 | 47.05 | A | N | |
| ATOM | 974 | C | ASN | A | 673 | 48.424 | 29.480 | −7.568 | 1.00 | 28.39 | A | C | |
| ATOM | 975 | O | ASN | A | 673 | 47.992 | 28.417 | −8.008 | 1.00 | 31.21 | A | O | |
| ATOM | 976 | N | PHE | A | 674 | 49.494 | 30.091 | −8.071 | 1.00 | 27.12 | A | N | |
| ATOM | 977 | CA | PHE | A | 674 | 50.242 | 29.519 | −9.184 | 1.00 | 25.22 | A | C | |
| ATOM | 978 | CB | PHE | A | 674 | 51.360 | 30.465 | −9.627 | 1.00 | 27.97 | A | C | |
| ATOM | 979 | CG | PHE | A | 674 | 52.221 | 29.917 | −10.733 | 1.00 | 27.02 | A | C | |
| ATOM | 980 | CD1 | PHE | A | 674 | 51.781 | 29.933 | −12.049 | 1.00 | 31.40 | A | C | |
| ATOM | 981 | CD2 | PHE | A | 674 | 53.470 | 29.374 | −10.455 | 1.00 | 29.05 | A | C | |
| ATOM | 982 | CE1 | PHE | A | 674 | 52.571 | 29.415 | −13.071 | 1.00 | 28.13 | A | C | |
| ATOM | 983 | CE2 | PHE | A | 674 | 54.264 | 28.856 | −11.468 | 1.00 | 34.83 | A | C | |
| ATOM | 984 | CZ | PHE | A | 674 | 53.812 | 28.877 | −12.781 | 1.00 | 25.40 | A | C | |
| ATOM | 985 | C | PHE | A | 674 | 50.855 | 28.215 | −8.688 | 1.00 | 29.92 | A | C | |
| ATOM | 986 | O | PHE | A | 674 | 50.774 | 27.184 | −9.352 | 1.00 | 29.40 | A | O | |
| ATOM | 987 | N | LEU | A | 675 | 51.465 | 28.267 | −7.509 | 1.00 | 25.02 | A | N | |
| ATOM | 988 | CA | LEU | A | 675 | 52.087 | 27.083 | −6.928 | 1.00 | 24.58 | A | C | |
| ATOM | 989 | CB | LEU | A | 675 | 52.742 | 27.429 | −5.592 | 1.00 | 25.53 | A | C | |
| ATOM | 990 | CG | LEU | A | 675 | 53.942 | 28.366 | −5.647 | 1.00 | 22.19 | A | C | |
| ATOM | 991 | CD1 | LEU | A | 675 | 54.363 | 28.726 | −4.223 | 1.00 | 28.72 | A | C | |
| ATOM | 992 | CD2 | LEU | A | 675 | 55.072 | 27.697 | −6.405 | 1.00 | 26.45 | A | C | |
| ATOM | 993 | C | LEU | A | 675 | 51.063 | 25.979 | −6.703 | 1.00 | 23.02 | A | C | |
| ATOM | 994 | O | LEU | A | 675 | 51.276 | 24.834 | −7.090 | 1.00 | 27.30 | A | O | |
| ATOM | 995 | N | ARG | A | 676 | 49.950 | .26.333 | −6.073 | 1.00 | 21.01 | A | N | |
| ATOM | 996 | CA | ARG | A | 676 | 48.910 | 25.370 | −5.781 | 1.00 | 24.79 | A | C | |
| ATOM | 997 | CB | ARG | A | 676 | 47.806 | 26.051 | −4.976 | 1.00 | 25.32 | A | C | |
| ATOM | 998 | CG | ARG | A | 676 | 48.312 | 26.448 | −3.603 | 1.00 | 28.64 | A | C | |
| ATOM | 999 | CD | ARG | A | 676 | 47.259 | 27.076 | −2.727 | 1.00 | 30.55 | A | C | |
| ATOM | 1000 | NE | ARG | A | 676 | 47.725 | 27.133 | −1.344 | 1.00 | 18.04 | A | N | |
| ATOM | 1001 | CZ | ARG | A | 676 | 46.981 | 27.541 | −0.326 | 1.00 | 27.36 | A | C | |
| ATOM | 1002 | NH1 | ARG | A | 676 | 45.737 | 27.937 | −0.539 | 1.00 | 28.80 | A | N | |
| ATOM | 1003 | NH2 | ARG | A | 676 | 47.470 | 27.528 | 0.906 | 1.00 | 26.04 | A | N | |
| ATOM | 1004 | C | ARG | A | 676 | 48.346 | 24.666 | −7.010 | 1.00 | 32.26 | A | C | |
| ATOM | 1005 | O | ARG | A | 676 | 47.958 | 23.503 | −6.934 | 1.00 | 31.67 | A | O | |
| ATOM | 1006 | N | ARG | A | 677 | 48.315 | 25.361 | −8.142 | 1.00 | 35.75 | A | N | |
| ATOM | 1007 | CA | ARC | A | 677 | 47.814 | 24.760 | −9.366 | 1.00 | 35.90 | A | C | |
| ATOM | 1008 | CB | ARG | A | 677 | 47.602 | 25.828 | −10.442 | 1.00 | 43.63 | A | C | |
| ATOM | 1009 | CG | ARG | A | 677 | 46.526 | 26.829 | −10.090 | 1.00 | 50.57 | A | C | |
| ATOM | 1010 | CD | ARG | A | 677 | 46.099 | 27.660 | −11.286 | 1.00 | 57.17 | A | C | |
| ATOM | 1011 | NE | ARG | A | 677 | 45.044 | 28.602 | −10.914 | 1.00 | 67.71 | A | N | |
| ATOM | 1012 | CZ | ARG | A | 677 | 44.352 | 29.340 | −11.778 | 1.00 | 71.08 | A | C | |
| ATOM | 1013 | NH1 | ARG | A | 677 | 44.596 | 29.248 | −13.080 | 1.00 | 71.07 | A | N | |
| ATOM | 1014 | NH2 | ARG | A | 677 | 43.415 | 30.171 | −11.337 | 1.00 | 67.48 | A | N | |
| ATOM | 1015 | C | ARG | A | 677 | 48.775 | 23.695 | −9.883 | 1.00 | 37.85 | A | C | |
| ATOM | 1016 | O | ARG | A | 677 | 48.349 | 22.619 | −10.299 | 1.00 | 35.60 | A | O | |
| ATOM | 1017 | N | LYS | A | 678 | 50.071 | 23.994 | −9.855 | 1.00 | 39.35 | A | N | |
| ATOM | 1018 | CA | LYS | A | 678 | 51.081 | 23.054 | −10.333 | 1.00 | 41.87 | A | C | |
| ATOM | 1019 | CB | LYS | A | 678 | 52.474 | 23.687 | −10.265 | 1.00 | 45.55 | A | C | |
| ATOM | 1020 | CG | LYS | A | 678 | 52.546 | 25.112 | −10.790 | 1.00 | 52.01 | A | C | |
| ATOM | 1021 | CD | LYS | A | 678 | 52.286 | 25.197 | −12.284 | 1.00 | 55.06 | A | C | |
| ATOM | 1022 | CB | LYS | A | 678 | 53.410 | 24.564 | −13.084 | 1.00 | 60.03 | A | C | |
| ATOM | 1023 | NZ | LYS | A | 678 | 53.232 | 24.817 | −14.540 | 1.00 | 62.90 | A | N | |
| ATOM | 1024 | C | LYS | A | 678 | 51.063 | 21.779 | −9.492 | 1.00 | 44.37 | A | C | |
| ATOM | 1025 | O | LYS | A | 678 | 51.073 | 20.667 | −10.027 | 1.00 | 40.32 | A | 0 | |
| ATOM | 1026 | N | SER | A | 679 | 51.037 | 21.950 | −8.174 | 1.00 | 41.30 | A | N | |
| ATOM | 1027 | CA | SER | A | 679 | 51.018 | 20.819 | −7.254 | 1.00 | 47.34 | A | C | |
| ATOM | 1028 | CB | SER | A | 679 | 50.965 | 21.310 | −5.808 | 1.00 | 47.51 | A | C | |
| ATOM | 1029 | OG | SER | A | 679 | 49.787 | 22.057 | −5.574 | 1.00 | 56.35 | A | O | |
| ATOM | 1030 | C | SER | A | 679 | 49.813 | 19.929 | −7.530 | 1.00 | 46.51 | A | C | |
| ATOM | 1031 | O | SER | A | 679 | 49.939 | 18.706 | −7.612 | 1.00 | 40.78 | A | O | |
| ATOM | 1032 | N | ARG | A | 680 | 48.645 | 20.547 | −7.663 | 1.00 | 42.11 | A | N | |
| ATOM | 1033 | CA | ARG | A | 680 | 47.433 | 19.798 | −7.946 | 1.00 | 47.66 | A | C | |
| ATOM | 1034 | CB | ARG | A | 680 | 46.249 | 20.747 | −8.141 | 1.00 | 51.99 | A | C | |
| ATOM | 1035 | CG | ARG | A | 680 | 44.979 | 20.044 | −8.588 | 1.00 | 59.20 | A | C | |
| ATOM | 1036 | CD | ARG | A | 680 | 44.473 | 19.092 | −7.518 | 1.00 | 60.66 | A | C | |
| ATOM | 1037 | NE | ARG | A | 680 | 43.610 | 18.056 | −8.078 | 1.00 | 69.98 | A | N | |
| ATOM | 1038 | CZ | ARG | A | 680 | 42.916 | 17.187 | −7.348 | 1.00 | 75.10 | A | C | |
| ATOM | 1039 | NH1 | ARG | A | 680 | 42.977 | 17.231 | −6.024 | 1.00 | 77.74 | A | N | |
| ATOM | 1040 | NH2 | ARG | A | 680 | 42.169 | 16.265 | −7.940 | 1.00 | 71.58 | A | N | |
| ATOM | 1041 | C | ARG | A | 680 | 47.666 | 18.995 | −9.222 | 1.00 | 49.29 | A | C | |
| ATOM | 1042 | O | ARG | A | 680 | 47.205 | 17.861 | −9.353 | 1.00 | 41.73 | A | O | |
| ATOM | 1043 | N | VAL | A | 681 | 48.399 | 19.595 | −10.156 | 1.00 | 49.39 | A | N | |
| ATOM | 1044 | CA | VAL | A | 681 | 48.705 | 18.951 | −11.423 | 1.00 | 50.39 | A | C | |
| ATOM | 1045 | CB | VAL | A | 681 | 49.300 | 19.952 | −12.427 | 1.00 | 48.40 | A | C | |
| ATOM | 1046 | CG1 | VAL | A | 681 | 49.678 | 19.240 | −13.713 | 1.00 | 55.59 | A | C | |
| ATOM | 1047 | CG2 | VAL | A | 681 | 48.298 | 21.050 | −12.715 | 1.00 | 54.90 | A | C | |
| ATOM | 1048 | C | VAL | A | 681 | 49.690 | 17.801 | −11.238 | 1.00 | 53.38 | A | C | |
| ATOM | 1049 | O | VAL | A | 681 | 49.628 | 16.812 | −11.969 | 1.00 | 52.52 | A | O | |
| ATOM | 1050 | N | LEU | A | 682 | 50.595 | 17.931 | −10.268 | 1.00 | 53.60 | A | N | |
| ATOM | 1051 | CA | LEU | A | 682 | 51.579 | 16.880 | −10.009 | 1.00 | 54.50 | A | C | |
| ATOM | 1052 | CB | LEU | A | 682 | 52.336 | 17.146 | −8.703 | 1.00 | 55.91 | A | C | |
| ATOM | 1053 | CG | LEU | A | 682 | 53.603 | 18.002 | −8.828 | 1.00 | 51.61 | A | C | |
| ATOM | 1054 | CD1 | LEU | A | 682 | 54.209 | 18.226 | −7.466 | 1.00 | 45.39 | A | C | |
| ATOM | 1055 | CD2 | LEU | A | 682 | 54.607 | 17.304 | −9.726 | 1.00 | 56.00 | A | C | |
| ATOM | 1056 | C | LEU | A | 682 | 50.893 | 15.521 | −9.956 | 1.00 | 58.22 | A | C | |
| ATOM | 1057 | O | LEU | A | 682 | 51.465 | 14.514 | −10.370 | 1.00 | 60.71 | A | O | |
| ATOM | 1058 | N | GLU | A | 683 | 49.670 | 15.487 | −9.436 | 1.00 | 61.1.00 | A | N | |
| ATOM | 1059 | CA | GLU | A | 683 | 48.920 | 14.239 | −9.407 | 1.00 | 67.33 | A | C | |
| ATOM | 1060 | CB | GLU | A | 683 | 47.752 | 14.315 | −8.423 | 1.00 | 65.47 | A | C | |
| ATOM | 1061 | CG | GLU | A | 683 | 48.135 | 14.069 | −6.969 | 1.00 | 69.77 | A | C | |
| ATOM | 1062 | CD | GLU | A | 683 | 48.555 | 15.326 | −6.236 | 1.00 | 68.58 | A | C | |
| ATOM | 1063 | OE1 | GLU | A | 683 | 47.734 | 16.262 | −6.135 | 1.00 | 70.57 | A | O | |
| ATOM | 1064 | OE2 | GLU | A | 683 | 49.704 | 15.375 | −5.752 | 1.00 | 71.66 | A | O | |
| ATOM | 1065 | C | GLU | A | 683 | 48.393 | 14.072 | −10.829 | 1.00 | 70.81 | A | C | |
| ATOM | 1066 | O | GLU | A | 683 | 47.208 | 13.817 | −11.046 | 1.007 | 1.00 | A | O | |
| ATOM | 1067 | N | THR | A | 684 | 49.307 | 14.233 | −11.785 | 1.00 | 74.11 | A | N | |
| ATOM | 1068 | CA | THR | A | 684 | 49.029 | 14.147 | −13.216 | 1.00 | 77.51 | A | C | |
| ATOM | 1069 | CB | THR | A | 684 | 50.337 | 13.995 | −14.022 | 1.00 | 78.20 | A | C | |
| ATOM | 107O | OG1 | THR | A | 684 | 51.394 | 13.570 | −13.151 | 1.00 | 76.25 | A | O | |
| ATOM | 1071 | CG2 | THR | A | 684 | 50.709 | 15.311 | −14.687 | 1.00 | 79.78 | A | C | |
| ATOM | 1072 | C | THR | A | 684 | 48.078 | 13.054 | −13.672 | 1.00 | 79.52 | A | C | |
| ATOM | 1073 | O | THR | A | 684 | 47.691 | 12.176 | −12.901 | 1.00 | 80.83 | A | O | |
| ATOM | 1074 | N | ASP | A | 685 | 47.711 | 13.126 | −14.949 | 1.00 | 80.38 | A | N | |
| ATOM | 1075 | CA | SP | A | 685 | 46.808 | 12.162 | −15.559 | 1.00 | 82.24 | A | C | |
| ATOM | 1076 | CB | ASP | A | 685 | 45.362 | 12.655 | −15.466 | 1.00 | 82.26 | A | C | |
| ATOM | 1077 | CG | ASP | A | 685 | 44.902 | 12.844 | −14.033 | 1.00 | 84.36 | A | C | |
| ATOM | 1078 | OD1 | ASP | A | 685 | 45.433 | 13.743 | −13.346 | 1.00 | 83.48 | A | O | |
| ATOM | 1079 | OD2 | ASP | A | 685 | 44.008 | 12.089 | −13.593 | 1.00 | 85.26 | A | O | |
| ATOM | 1080 | C | ASP | A | 685 | 47.183 | 11.950 | −17.022 | 1.00 | 84.72 | A | C | |
| ATOM | 1081 | O | ASP | A | 685 | 48.259 | 12.357 | −17.464 | 1.00 | 87.50 | A | O | |
| ATOM | 1082 | N | SER | A | 693 | 53.014 | 22.633 | −18.624 | 1.00 | 70.15 | A | N | |
| ATOM | 1083 | CA | SER | A | 693 | 54.049 | 23.336 | −17.873 | 1.00 | 72.82 | A | C | |
| ATOM | 1084 | CB | SER | A | 693 | 53.649 | 24.804 | −17.684 | 1.00 | 73.91 | A | C | |
| ATOM | 1085 | OG | SER | A | 693 | 52359 | 24.918 | −17.105 | 1.00 | 75.91 | A | O | |
| ATOM | 1086 | C | SER | A | 693 | 54.289 | 22.678 | −16.513 | 1.00 | 73.06 | A | C | |
| ATOM | 1087 | O | SER | A | 693 | 53.355 | 22.491 | −15.730 | 1.00 | 75.58 | A | O | |
| ATOM | 1088 | N | THR | A | 694 | 55.545 | 22.331 | −16.239 | 1.00 | 68.76 | A | N | |
| ATOM | 1089 | CA | THR | A | 694 | 55.908 | 21.684 | −14.981 | 1.00 | 66.40 | A | C | |
| ATOM | 1090 | CB | THR | A | 694 | 56;243 | 20.197 | −15.204 | 1.00 | 69.60 | A | C | |
| ATOM | 1091 | OG1 | THR | A | 694 | 55.076 | 19.509 | −15.670 | 1.00 | 70.35 | A | O | |
| ATOM | 1092 | CG2 | THR | A | 694 | 56.726 | 19.559 | −13.908 | 1.00 | 70.77 | A | C | |
| ATOM | 1093 | C | THR | A | 694 | 57.102 | 22.354 | −14.304 | 1.00 | 60.92 | A | C | |
| ATOM | 1094 | O | THR | A | 694 | 58.222 | 22.321 | −14.816 | 1.00 | 58.71 | A | O | |
| ATOM | 1095 | N | ALA | A | 697 | 56.854 | 22.948 | −13.141 | 1.00 | 55.19 | A | N | |
| ATOM | 1096 | CA | ALA | A | 697 | 57.895 | 23.639 | −12.391 | 1.00 | 48.73 | A | C | |
| ATOM | 1097 | CB | ALA | A | 697 | 57.335 | 24.124 | −11.052 | 1.00 | 45.94 | A | C | |
| ATOM | 1098 | C | ALA | A | 697 | 59.128 | 22.773 | −12.157 | 1.00 | 47.24 | A | C | |
| ATOM | 1099 | O | ALA | A | 697 | 59.028 | 21.579 | −11.877 | 1.00 | 46.79 | A | O | |
| ATOM | 1100 | N | SER | A | 698 | 60.294 | 23.393 | −12.280 | 1.00 | 44.27 | A | N | |
| ATOM | 1101 | CA | SER | A | 698 | 61.562 | 22.712 | −12.070 | 1.00 | 43.79 | A | C | |
| ATOM | 1102 | CB | SER | A | 698 | 62.424 | 22.802 | −13.327 | 1.00 | 49.31 | A | C | |
| ATOM | 1103 | CG | SER | A | 698 | 63.692 | 22.204 | −13.120 | 1.00 | 57.76 | A | O | |
| ATOM | 1104 | C | SER | A | 698 | 62.274 | 23.396 | −10.909 | 1.00 | 42.26 | A | C | |
| ATOM | 1105 | O | SER | A | 698 | 61.895 | 24.496 | −10.504 | 1.00 | 35.95 | A | O | |
| ATOM | 1106 | N | THR | A | 699 | 63.305 | 22.751 | −10.375 | 1.00 | 40.48 | A | N | |
| ATOM | 1107 | CA | THR | A | 699 | 64.042 | 23.331 | −9.262 | 1.00 | 41.25 | A | C | |
| ATOM | 1108 | CB | THR | A | 699 | 65.246 | 22.443 | −8.860 | 1.00 | 43.33 | A | C | |
| ATOM | 1109 | OG1 | THR | A | 699 | 65.943 | 23.050 | −7.763 | 1.00 | 54.42 | A | O | |
| ATOM | 1110 | CG2 | THR | A | 699 | 66.199 | 22.262 | −10.032 | 1.00 | 51.95 | A | C | |
| ATOM | 1111 | C | THR | A | 699 | 64.525 | 24.735 | −9.628 | 1.00 | 40.03 | A | C | |
| ATOM | 1112 | O | THR | A | 699 | 64.577 | 25.624 | −8.775 | 1.00 | 33.42 | A | O | |
| ATOM | 1113 | N | ARG | A | 753 | 64.863 | 24.935 | −10.900 | 1.00 | 32.66 | A | N | |
| ATOM | 1114 | CA | ARG | A | 753 | 65.326 | 26.236 | −11.370 | 1.00 | 38.55 | A | C | |
| ATOM | 1115 | CB | ARG | A | 753 | 65.785 | 26.140 | −12.824 | 1.00 | 42.14 | A | C | |
| ATOM | 1116 | CG | ARG | A | 753 | 64.680 | 25.707 | −13.766 | 1.00 | 48.56 | A | C | |
| ATOM | 1117 | CD | ARG | A | 753 | 65.162 | 25.606 | −15.202 | 1.00 | 58.62 | A | C | |
| ATOM | 1118 | NE | ARG | A | 753 | 64.151 | 24.982 | −16.050 | 1.00 | 67.47 | A | N | |
| ATOM | 1119 | CZ | ARG | A | 753 | 64.294 | 24.769 | −17.353 | 1.00 | 70.46 | A | C | |
| ATOM | 1120 | NH1 | ARG | A | 753 | 65.412 | 25.132 | −17.965 | 1.00 | 74.39 | A | N | |
| ATOM | 1121 | NH2 | ARG | A | 753 | 63.320 | 24.191 | −18.044 | 1.00 | 73.59 | A | N | |
| ATOM | 1122 | C | ARG | A | 753 | 64.192 | 27.257 | −11.269 | 1.00 | 36.15 | A | C | |
| ATOM | 1123 | O | ARG | A | 753 | 64.419 | 28.427 | −10.958 | 1.00 | 31.25 | A | O | |
| ATOM | 1124 | N | ASP | A | 754 | 62.975 | 26.804 | −11.545 | 1.00 | 30.98 | A | N | |
| ATOM | 1125 | CA | ASP | A | 754 | 61.797 | 27.661 | −11.492 | 1.00 | 31.57 | A | C | |
| ATOM | 1126 | CB | ASP | A | 754 | 60.572 | 26.884 | −11.977 | 1.00 | 34.21 | A | C | |
| ATOM | 1127 | CG | ASP | A | 754 | 60.674 | 26.497 | −13.441 | 1.00 | 33.79 | A | C | |
| ATOM | 1128 | OD1 | ASP | A | 754 | 60.068 | 25.476 | −13.836 | 1.00 | 36.78 | A | O | |
| ATOM | 1129 | OD2 | ASP | A | 754 | 61.352 | 27.224 | −14.199 | 1.00 | 35.61 | A | O | |
| ATOM | 1130 | C | ASP | A | 75.4 | 61.557 | 28.168 | −10.074 | 1.00 | 31.79 | A | C | |
| ATOM | 1131 | O | ASP | A | 754 | 61.257 | 29.345 | −9.870 | 1.00 | 28.70 | A | O | |
| ATOM | 1132 | N | LEU | A | 755 | 61.699 | 27.277 | −9.099 | 1.00 | 29.81 | A | N | |
| ATOM | 1133 | CA | LEU | A | 755 | 61.489 | 27.637 | −7.699 | 1.00 | 24.86 | A | C | |
| ATOM | 1134 | CB | LEU | A | 755 | 61.412 | 26.376 | −6.834 | 1.00 | 25.77 | A | C | |
| ATOM | 1135 | CG | LEU | A | 755 | 60.349 | 25.348 | −7.245 | 1.00 | 23.84 | A | C | |
| ATOM | 1136 | CD1 | LEU | A | 755 | 60.342 | 24.202 | −6.240 | 1.00 | 25.93 | A | C | |
| ATOM | 1137 | CD2 | LEU | A | 755 | 58.984 | 26.001 | −7.332 | 1.00 | 21.61 | A | C | |
| ATOM | 1138 | C | LEU | A | 755 | 62.616 | 28.531 | −7.210 | 1.00 | 26.51 | A | C | |
| ATOM | 1139 | O | LEU | A | 755 | 62.401 | 29.426 | −6.391 | 1.00 | 26.03 | A | O | |
| ATOM | 1140 | N | LEU | A | 756 | 63.823 | 28.284 | −7.712 | 1.00 | 19.75 | A | N | |
| ATOM | 1141 | CA | LEU | A | 756 | 64.978 | 29.081 | −7.329 | 1.00 | 25.64 | A | C | |
| ATOM | 1142 | CB | LEU | A | 756 | 66.267 | 28.449 | −7.862 | 1.00 | 19.69 | A | C | |
| ATOM | 1143 | CG | LEU | A | 756 | 66.699 | 27.143 | −7.177 | 1.00 | 24.24 | A | C | |
| ATOM | 1144 | CD1 | LEU | A | 756 | 67.899 | 26.553 | −7.91.1 | 1.00 | 29.63 | A | C | |
| ATOM | 1145 | CD2 | LEU | A | 756 | 67.054 | 27.409 | −5.719 | 1.00 | 25.25 | A | C | |
| ATOM | 1146 | C | LEU | A | 756 | 64.834 | 30.504 | −7.848 | 1.00 | 23.89 | A | C | |
| ATOM | 1147 | O | LEU | A | 756 | 65.181 | 31.455 | −7.155 | 1.00 | 21.64 | A | O | |
| ATOM | 1148 | N | HIS | A | 757 | 64.312 | 30.649 | −9.064 | 1.00 | 26.01 | A | N | |
| ATOM | 1149 | CA | HIS | A | 757 | 64.122 | 31.969 | −9.658 | 1.00 | 32.21A | | C | |
| ATOM | 1150 | CB | HIS | A | 757 | 63.680 | 31.827 | −11.117 | 1.00 | 30.34 | A | C | |
| ATOM | 1151 | CG | HIS | A | 757 | 64.748 | 31.276 | −12.010 | 1.00 | 43.72 | A | C | |
| ATOM | 1152 | CD2 | HIS | A | 757 | 66.018 | 30.888 | −11.744 | 1.00 | 47.21 | A | C | |
| ATOM | 1153 | ND1 | HIS | A | 757 | 64.561 | 31.070 | −13.360 | 1.00 | 47.06 | A | N | |
| ATOM | 1154 | CE1 | HIS | A | 757 | 65.668 | 30.580 | −13.888 | 1.00 | 46.10 | A | C | |
| ATOM | 1155 | NE2 | HIS | A | 757 | 66.568 | 30.459 | −12.928 | 1.00 | 50.20 | A | N | |
| ATOM | 1156 | C | HIS | A | 757 | 63.103 | 32.784 | −8.861 | 1.00 | 32.11 | A | C | |
| ATOM | 1157 | O | HIS | A | 757 | 63.314 | 33.971 | −8.589 | 1.00 | 28.26 | A | O | |
| ATOM | 1158 | N | PHE | A | 758 | 62.000 | 32.146 | −8.487 | 1.00 | 26.33 | A | N | |
| ATOM | 1159 | CA | PHE | A | 758 | 60.982 | 32.823 | −7.696 | 1.00 | 26.92 | A | C | |
| ATOM | 1160 | CB | PHE | A | 758 | 59.850 | 31.864 | −7.345 | 1.00 | 19.40 | A | C | |
| ATOM | 1161 | CG | PHE | A | 758 | 58.960 | 31.528 | −8.505 | 1.00 | 32.48 | A | C | |
| ATOM | 1162 | CD1 | PHE | A | 758 | 58.233 | 30.346 | −8.519 | 1.00 | 29.63 | A | C | |
| ATOM | 1163 | CD2 | PHE | A | 758 | 58.834 | 32.402 | −9.578 | 1.00 | 27.05 | A | C | |
| ATOM | 1164 | CE1 | PHE | A | 758 | 57.398 | 30.039 | −9.579 | 1.00 | 30.08 | A | C | |
| ATOM | 1165 | CE2 | PHE | A | 758 | 57.999 | 32.102 | −10.642 | 1.00 | 26.86 | A | C | |
| ATOM | 1166 | CZ | PHE | A | 758 | 57.282 | 30.922 | −10.644 | 1.00 | 23.91 | A | C | |
| ATOM | 1167 | C | PHE | A | 758 | 61.640 | 33.319 | −6.418 | 1.00 | 24.29 | A | C | |
| ATOM | 1168 | O | PHE | A | 758 | 61486 | 34.474 | −6.048 | 1.00 | 15.30 | A | O | |
| ATOM | 1169 | N | SER | A | 759 | 62.388 | 32.428 | −5.771 | 1.00 | 25.71 | A | N | |
| ATOM | 1170 | CA | SER | A | 759 | 63.081 | 32.722 | −4.522 | 1.00 | 23.10 | A | C | |
| ATOM | 1171 | CB | SER | A | 759 | 63.796 | 31.467 | −4.011 | 1.00 | 22.51 | A | C | |
| ATOM | 1172 | OG | SER | A | 759 | 62.916 | 30.360 | −3.979 | 1.00 | 17.85 | A | O | |
| ATOM | 1173 | C | SER | A | 759 | 64.086 | 33.852 | −4.682 | 1.00 | 21.83 | A | C | |
| ATOM | 1174 | O | SER | A | 759 | 64.210 | 34.707 | −3.803 | 1.00 | 20.90 | A | O | |
| ATOM | 1175 | N | SER | A | 760 | 64.807 | 33.849 | −5.802 | 1.00 | 15.60 | A | N | |
| ATOM | 1176 | CA | SER | A | 760 | 65.801 | 34.872 | −6.096 | 1.00 | 18.74 | A | C | |
| ATOM | 1177 | CB | SER | A | 760 | 66.625 | 34.471 | −7.325 | 1.00 | 20.47 | A | C | |
| ATOM | 1178 | OG | SER | A | 760 | 67.693 | 33.625 | −6.957 | 1.00 | 26.56 | A | O | |
| ATOM | 1179 | C | SER | A | 760 | 65.171 | 36.237 | −6.346 | 1.00 | 18.72 | A | C | |
| ATOM | 1180 | O | SER | A | 760 | 65.590 | 37.240 | −5.767 | 1.00 | 20.41 | A | O | |
| ATOM | 1181 | N | GLN | A | 761 | 64.172 | 36.259 | −7.222 | 1.00 | 16.97 | A | N | |
| ATOM | 1182 | CA | GLN | A | 761 | 63.459 | 37.477 | −7.581 | 1.00 | 18.08 | A | C | |
| ATOM | 1183 | CB | GLN | A | 761 | 62.336 | 37.132 | −8.554 | 1.00 | 21.99 | A | C | |
| ATOM | 1184 | CG | GLN | A | 761 | 62.839 | 37.017 | −9.991 | 1.00 | 29.71 | A | C | |
| ATOM | 1185 | CD | GLN | A | 761 | 62.167 | 35.912 | −10.781 | 1.00 | 25.33 | A | C | |
| ATOM | 1186 | OE1 | GLN | A | 761 | 60.998 | 35.599 | −10.568 | 1.00 | 26.00 | A | O | |
| ATOM | 1187 | NE2 | GLN | A | 761 | 62.905 | 35.327 | −11.718 | 1.00 | 29.88 | A | N | |
| ATOM | 1188 | C | GLN | A | 761 | 62.911 | 38.182 | −6.349 | 1.00 | 23.40 | A | C | |
| ATOM | 1189 | O | GLN | A | 761 | 63.090 | 39.390 | 6.183 | 1.00 | 21.31 | A | O | |
| ATOM | 1190 | N | VAL | A | 762 | 62.260 | 37.420 | −5.479 | 1.00 | 20.06 | A | N | |
| ATOM | 1191 | CA | VAL | A | 762 | 61.720 | 37.984 | −4.255 | 1.00 | 20.11 | A | C | |
| ATOM | 1192 | CB | VAL | A | 762 | 60.819 | 36.956 | −3.527 | 1.00 | 26.81 | A | C | |
| ATOM | 1193 | CG1 | VAL | A | 762 | 60.373 | 37.498 | 2.188 | 1.00 | 11.82 | A | C | |
| ATOM | 1194 | CG2 | VAL | A | 762 | 59.598 | 36.654 | 4.377 | 1.00 | 17.27 | A | C | |
| ATOM | 1195 | C | VAL | A | 762 | 62.857 | 38.444 | −3.334 | 1.00 | 19.95 | A | C | |
| ATOM | 1196 | O | VAL | A | 762 | 62.780 | 39.520 | −2.742 | 1.00 | 13.43 | A | O | |
| ATOM | 1197 | N | ALA | A | 763 | 63.921 | 37.645 | −3.232 | 1.00 | 18.45 | A | N | |
| ATOM | 1198 | CA | ALA | A | 763 | 65.058 | 37.990 | −2.381 | 1.00 | 22.30 | A | C | |
| ATOM | 1199 | CB | ALA | A | 763 | 66.093 | 36.862 | −2.388 | 1.00 | 18.28 | A | C | |
| ATOM | 1200 | C | ALA | A | 763 | 65.689 | 39.292 | −2.861 | 1.00 | 23.40 | A | C | |
| ATOM | 1201 | O | ALA | A | 76.3 | 66.188 | 40.082 | −2.061 | 1.00 | 15.76 | A | O | |
| ATOM | 1202 | N | GLN | A | 764 | 65.667 | 39.501 | −4.175 | 1.00 | 21.55 | A | N | |
| ATOM | 1203 | CA | GLN | A | 764 | 66.202 | 40.713 | 4.787 | 1.00 | 27.04 | A | C | |
| ATOM | 1204 | CB | GLN | A | 764 | 66.286 | 40.548 | −6.308 | 1.00 | 24.90 | A | C | |
| ATOM | 1205 | CG | GLN | A | 764 | 67.565 | 39.906 | −6.810 | 1.00 | 31.00 | A | C | |
| ATOM | 1206 | CD | GLN | A | 764 | 67.407 | 39.343 | −8.211 | 1.00 | 46.06 | A | C | |
| ATOM | 1207 | OE1 | GLN | A | 764 | 66.908 | 40.019 | 9.113 | 1.00 | 55.63 | A | O | |
| ATOM | 1208 | NE2 | GN | A | 764 | 67.835 | 38.098 | −8.400 | 1.00 | 53.80 | A | N | |
| ATOM | 1209 | C | GLN | A | 764 | 65.300 | 41.901 | −4.451 | 1.00 | 20.31 | A | C | |
| ATOM | 1210 | O | GLN | A | 764 | 65.786 | 42.977 | −4.108 | 1.00 | 21.11 | A | O | |
| ATOM | 1211 | N | GLY | A | 765 | 63.990 | 41.697 | −4.544 | 1.00 | 16.15 | A | N | |
| ATOM | 1212 | CA | GLY | A | 765 | 63.056 | 42.766 | 4.225 | 1.00 | 17.64 | A | C | |
| ATOM | 1213 | C | GLY | A | 765 | 63.248 | 43.179 | −2.772 | 1.00 | 16.42 | A | C | |
| ATOM | 1214 | O | GLY | A | 765 | 63.328 | 44.363 | −2.455 | 1.00 | 19.02 | A | O | |
| ATOM | 1215 | N | MET | A | 766 | 63.357 | 42.188 | −1.893 | 1.00 | 15.31 | A | N | |
| ATOM | 1216 | CA | MET | A | 766 | 63.550 | 42.437 | −0.457 | 1.00 | 14.10 | A | C | |
| ATOM | 1217 | CB | MET | A | 766 | 63.409 | 41.130 | 0.316 | 1.00 | 16.14 | A | C | |
| ATOM | 1218 | CG | MET | A | 766 | 61.983 | 40.573 | 0.342 | 1.00 | 19.94 | A | C | |
| ATOM | 1219 | SD | MET | A | 766 | 60.729 | 41.744 | 0.929 | 1.00 | 19.78 | A | S | |
| ATOM | 1220 | CE | MET | A | 766 | 61.141 | 41.808 | 2.738 | 1.00 | 13.84 | A | C | |
| ATOM | 1221 | C | MET | A | 766 | 64.907 | 43.080 | −0.166 | 1.00 | 17.88 | A | C | |
| ATOM | 1222 | O | MET | A | 766 | 65.044 | 43.905 | 0.743 | 1.00 | 13.94 | A | O | |
| ATOM | 1223 | N | ALA | A | 7.67 | 65.916 | 42.701 | −0.940 | 1.00 | 17.98 | A | N | |
| ATOM | 1224 | CA | ALA | A | 767 | 67.237 | 43.272 | −0.770 | 1.00 | 19.33 | A | C | |
| ATOM | 1225 | CB | ALA | A | 767 | 68.228 | 42.579 | −1.700 | 1.00 | 21.62 | A | C | |
| ATOM | 1226 | C | ALA | A | 767 | 67.135 | 44.766 | 1.109 | 1.00 | 17.05 | A | C | |
| ATOM | 1227 | O | ALA | A | 767 | 67.737 | 45.598 | −0.450 | 1.00 | 14.87 | A | O | |
| ATOM | 1228 | N | PHE | A | 768 | 66.371 | 45.090 | 2.148 | 1.00 | 18.04 | A | N | |
| ATOM | 1229 | CA | PHE | A | 768 | 66.188 | 46.479 | −2.561 | 1.00 | 21.12 | A | C | |
| ATOM | 1230 | CB | PHE | A | 768 | 65.348 | 46.534 | −3.835 | 1.00 | 19.56 | A | C | |
| ATOM | 1231 | CG | PHE | A | 768 | 65.109 | 47.932 | −4.353 | 1.00 | 21.17 | A | C | |
| ATOM | 1232 | CD1 | PHE | A | 768 | 66.164 | 48.714 | −4.793 | 1.00 | 24.80 | A | C | |
| ATOM | 1233 | CD2 | PHE | A | 768 | 63.828 | 48.461 | 4.393 | 1.00 | 27.04 | A | C | |
| ATOM | 1234 | CE1 | PHE | A | 768 | 65.947 | 49.998 | −5.264 | 1.00 | 27.09 | A | C | |
| ATOM | 1235 | CE2 | PHE | A | 768 | 63.606 | 49.748 | −4.864 | 1.00 | 25.74 | A | C | |
| ATOM | 1236 | CZ | PHE | A | 768 | 64.671 | 50.513 | −5.299 | 1.00 | 22.52 | A | C | |
| ATOM | 1237 | C | PHE | A | 768 | 65.500 | 47.271 | −1.444 | 1.00 | 21.76 | A | C | |
| ATOM | 1238 | O | PHE | A | 768 | 65.944 | 48.355 | −1.063 | 1.00 | 23.66 | A | O | |
| ATOM | 1239 | N | LEU | A | 769 | 64.419 | 46.716 | −0.913 | 1.00 | 20.97 | A | N | |
| ATOM | 1240 | CA | LEU | A | 769 | 63.680 | 47.367 | 0.156 | 1.00 | 19.58 | A | C | |
| ATOM | 1241 | CB | LEU | A | 769 | 62.482 | 46.502 | 0.584 | 1.00 | 19.74 | A | C | |
| ATOM | 1242 | CG | LEU | A | 769 | 61.328 | 46.311 | −0.414 | 1.00 | 22.31 | A | C | |
| ATOM | 1243 | CD1 | LEU | A | 769 | 60.115 | 45.707 | 0.283 | 1.00 | 16.09 | A | C | |
| ATOM | 1244 | CD2 | LEU | A | 769 | 60.941 | 47.649 | −1.002 | 1.00 | 21.21 | A | C | |
| ATOM | 1245 | C | LEU | A | 769 | 64.593 | 47.621 | 1.350 | 1.00 | 18.68 | A | C | |
| ATOM | 1246 | O | LEU | A | 769 | 64.631 | 48.730 | 1.898 | 1.00 | 17.86 | A | O | |
| ATOM | 1247 | N | ALA | A | 770 | 65.335 | 46.590 | 1.744 | 1.00 | 20.17 | A | N | |
| ATOM | 1248 | CA | ALA | A | 770 | 66.245 | 46.691 | 2.878 | 1.00 | 24.54 | A | C | |
| ATOM | 1249 | CB | ALA | A | 770 | 66.937 | 45.352 | 3.104 | 1.00 | 24.53 | A | C | |
| ATOM | 1250 | C | ALA | A | 770 | 67289 | 47.799 | 2.705 | 1.00 | 23.83 | A | C | |
| ATOM | 1251 | O | ALA | A | 770 | 67.674 | 48.455 | 3.678 | 1.00 | 24.25 | A | O | |
| ATOM | 1252 | N | SER | A | 771 | 67.743 | 48.011 | 1.472 | 1.00 | 23.09 | A | N | |
| ATOM | 1253 | CA | SER | A | 771 | 68.741 | 49.038 | 1.209 | 1.00 | 25.52 | A | C | |
| ATOM | 1254 | CB | SER | A | 771 | 69.338 | 48.864 | −0.193 | 1.00 | 21.60 | A | C | |
| ATOM | 1255 | OG | SER | A | 771 | 68.416 | 49.248 | −1.194 | 1.00 | 21.11 | A | O | |
| ATOM | 1256 | C | SER | A | 771 | 68.129 | 50.433 | 1.352 | 1.00 | 26.48 | A | C | |
| ATOM | 1257 | O | SER | A | 771 | 68.843 | 51.422 | 1.501 | 1.00 | 26.76 | A | O | |
| ATOM | 1258 | N | LYS | A | 772 | 66.802 | 50.504 | 1.315 | 1.00 | 23.57 | A | N | |
| ATOM | 1259 | CA | LYS | A | 772 | 66.097 | 51.773 | 1.455 | 1.00 | 26.01 | A | C | |
| ATOM | 1260 | CB | LYS | A | 772 | 64.931 | 51.853 | 0.470 | 1.00 | 26.88 | A | C | |
| ATOM | 1261 | CG | LYS | A | 772 | 65.288 | 51.585 | −0.985 | 1.00 | 22.86 | A | C | |
| ATOM | 1262 | CD | LYS | A | 772 | 66.232 | 52.637 | −1.529 | 1.00 | 29.15 | A | C | |
| ATOM | 1263 | CE | LYS | A | 772 | 66.566 | 52.363 | −2.983 | 1.00 | 35.07 | A | C | |
| ATOM | 1264 | NZ | LYS | A | 772 | 67.207 | 53.534 | −3.628 | 1.00 | 39.67 | A | N | |
| ATOM | 1265 | C | LYS | A | 772 | 65.540 | 51.867 | 2.872 | 1.00 | 21.18 | A | C | |
| ATOM | 1266 | O | LYS | A | 772 | 64.763 | 52.764 | 3.181 | 1.00 | 25.40 | A | O | |
| ATOM | 1267 | N | ASN | A | 773 | 65.942 | 50.931 | 3.725 | 1.00 | 24.24 | A | N | |
| ATOM | 1268 | CA | ASN | A | 773 | 65.451 | 50.880 | 5.097 | 1.00 | 22.74 | A | C | |
| ATOM | 1269 | CB | ASN | A | 773 | 65.915 | 52.100 | 5.899 | 1.00 | 33.02 | A | C | |
| ATOM | 1270 | CG | ASN | A | 773 | 67.414 | 52.133 | 6.087 | 1.00 | 37.25 | A | C | |
| ATOM | 1271 | OD1 | ASN | A | 773 | 68.041 | 51.102 | 6.334 | 1.00 | 42.18 | A | O | |
| ATOM | 1272 | ND2 | ASN | A | 773 | 67.999 | 53.322 | 5.984 | 1.00 | 37.80 | A | N | |
| ATOM | 1273 | C | ASN | A | 773 | 63.931 | 50.809 | 5.096 | 1.00 | 22.96 | A | C | |
| ATOM | 1274 | O | ASN | A | 773 | 63.263 | 51.461 | 5.892 | 1.00 | 24.43 | A | O | |
| ATOM | 1275 | N | CYS | A | 774 | 63.384 | 50.007 | 4.189 | 1.00 | 20.83 | A | N | |
| ATOM | 1276 | CA | CYS | A | 774 | 61.940 | 49.848 | 4.101 | 1.00 | 20.83 | A | C | |
| ATOM | 1277 | CB | CYS | A | 774 | 61.456 | 50.089 | 2.662 | 1.00 | 23.20 | A | C | |
| ATOM | 1278 | SG | CYS | A | 774 | 59.642 | 50.006 | 2.440 | 1.00 | 20.10 | A | S | |
| ATOM | 1279 | C | CYS | A | 774 | 61.545 | 48.438 | 4.527 | 1.00 | 21.51 | A | C | |
| ATOM | 1280 | O | CYS | A | 774 | 62.047 | 47.462 | 3.976 | 1.00 | 20.46 | A | O | |
| ATOM | 1281 | N | ILE | A | 77Sf | 60.667 | 48.332 | 5.521 | 1.00 | 19.62 | A | N | |
| ATOM | 1282 | CA | ILE | A | 775 | 60.203 | 47.022 | 5.950 | 1.00 | 24.15 | A | C | |
| ATOM | 1283 | CB | ILE | A | 775 | 60.013 | 46.938 | 7.478 | 1.00 | 23.71 | A | C | |
| ATOM | 1284 | CG2 | ILE | A | 775 | 61.356 | 47.100 | 8.173 | 1.00 | 33.09 | A | C | |
| ATOM | 1285 | CG1 | ILE | A | 775 | 59.035 | 48.002 | 7.953 | 1.00 | 25.50 | A | C | |
| ATOM | 1286 | CD1 | ILE | A | 775 | 58.639 | 47.831 | 9.388 | 1.00 | 31.05 | A | C | |
| ATOM | 1287 | C | ILE | A | 775 | 58.887 | 46.706 | 5.244 | 1.00 | 20.48 | A | C | |
| ATOM | 1288 | O | ILE | A | 775 | 58.079 | 47.599 | 4.967 | 1.00 | 20.87 | A | O | |
| ATOM | 1289 | N | HIS | A | 776 | 58.694 | 45.426 | 4.946 | 1.00 | 19.40 | A | N | |
| ATOM | 1290 | CA | HIS | A | 776 | 57.521 | 44.93 | 4.233 | 1.00 | 21.94 | A | C | |
| ATOM | 1291 | CB | HIS | A | 776 | 57.929 | 43.668 | 3.464 | 1.00 | 20.79 | A | C | |
| ATOM | 1292 | CG | HIS | A | 776 | 56.887 | 43.150 | 2.523 | 1.00 | 21.62 | A | C | |
| ATOM | 1293 | CD2 | HIS | A | 776 | 56.877 | 43.060 | 1.171 | 1.00 | 14.45 | A | C | |
| ATOM | 1294 | ND1 | HIS | A | 776 | 55.697 | 42.604 | 2.952 | 1.00 | 16.21 | A | N | |
| ATOM | 1295 | CE1 | HIS | A | 776 | 54.999 | 42.198 | 1.905 | 1.00 | 16.39 | A | C | |
| ATOM | 1296 | NE2 | HIS | A | 776 | 55.693 | 42.464 | 0.812 | 1.00 | 13.85 | A | N | |
| ATOM | 1297 | C | HIS | A | 776 | 56.394 | 44.617 | 5.210 | 1.00 | 17.18 | A | C | |
| ATOM | 1298 | O | HIS | A | 776 | 55.285 | 45.137 | 5.092 | 1.00 | 20.21 | A | O | |
| ATOM | 1299 | N | ARG | A | 777 | 56.704 | 43.740 | 6.154 | 1.00 | 19.26 | A | N | |
| ATOM | 1300 | CA | ARG | A | 777 | 55.798 | 43.313 | 7.214 | 1.00 | 21.85 | A | C | |
| ATOM | 1301 | CB | ARG | A | 777 | 55.177 | 44.535 | 7.902 | 1.00 | 25.43 | A | C | |
| ATOM | 1302 | CG | ARG | A | 777 | 56.206 | 45.320 | 8.708 | 1.00 | 30.35 | A | C | |
| ATOM | 1303 | CD | ARG | A | 777 | SS.569 | 46.314 | 9.665 | 1.00 | 31.32 | A | C | |
| ATOM | 1304 | NE | ARG | A | 777 | 54.951 | 47.447 | 8.984 | 1.00 | 31.09 | A | N | |
| ATOM | 1305 | CZ | ARC | A | 777 | 54.412 | 48.482 | 9.622 | 1.00 | 38.89 | A | C | |
| ATOM | 1306 | NH1 | ARG | A | 777 | 54.423 | 48.507 | 10.948 | 1.00 | 27.20 | A | N | |
| ATOM | 1307 | NH2 | ARG | A | 777 | 53.872 | 49.491 | 8.943 | 1.00 | 25.00 | A | N | |
| ATOM | 1308 | C | ARG | A | 777 | 54.724 | 42.282 | 6.889 | 1.00 | 19.94 | A | C | |
| ATOM | 1309 | O | ARG | A | 777 | 54.049 | 41.814 | 7.792 | 1.00 | 22.92 | A | 0 | |
| ATOM | 1310 | N | ASP | A | 778 | 54.553 | 41.926 | 5.615 | 1.00 | 16.40 | A | N | |
| ATOM | 1311 | CA | ASP | A | 778 | 53.573 | 40.891 | 5.264 | 1.00 | 16.07 | A | C | |
| ATOM | 1312 | CB | ASP | A | 778 | 52.256 | 41.502 | 4.751 | 1.00 | 16.31 | A | C | |
| ATOM | 1313 | CG | ASP | A | 778 | 51.059 | 40.545 | 4.899 | 1.00 | 19.62 | A | C | |
| ATOM | 1314 | OD1 | ASP | A | 778 | 49.921 | 40.974 | 4.613 | 1.00 | 17.04 | A | O | |
| ATOM | 1315 | OD2 | ASP | A | 778 | 51.244 | 39.371 | 5.310 | 1.00 | 14.91 | A | O | |
| ATOM | 1316 | C | ASP | A | 778 | 54.141 | 39.965 | 4.194 | 1.00 | 15.26 | A | C | |
| ATOM | 1317 | O | ASP | A | 778 | 53.459 | 39.648 | 3.218 | 1.00 | 18.58 | A | O | |
| ATOM | 1318 | N | VAL | A | 779 | 55.385 | 39.531 | 4.371 | 1.00 | 14.29 | A | N | |
| ATOM | 1319 | CA | VAL | A | 779 | 55.993 | 38.631 | 3.403 | 1.00 | 21.63 | A | C | |
| ATOM | 1320 | CB | VAL | A | 779 | 57.506 | 38.461 | 3.658 | 1.00 | 20.49 | A | C | |
| ATOM | 1321 | CG1 | VAL | A | 779 | 58.078 | 37.497 | 2.646 | 1.00 | 8.30 | A | C | |
| ATOM | 1322 | CG2 | VAL | A | 779 | 58.223 | 39.828 | 3.571 | 1.00 | 20.35 | A | C | |
| ATOM | 1323 | C | VAL | A | 779 | 55.291 | 37.266 | 3.477 | 1.00 | 15.56 | A | C | |
| ATOM | 1324 | O | VAL | A | 779 | 55.250 | 36.630 | 4.529 | 1.00 | 18.67 | A | O | |
| ATOM | 1325 | N | ALA | A | 780 | 54.728 | 36.839 | 2.351 | 1.00 | 11.25 | A | N | |
| ATOM | 1326 | CA | ALA | A | 780 | 54.005 | 35.575 | 2.252 | 1.00 | 9.85 | A | C | |
| ATOM | 1327 | CB | ALA | A | 780 | 52.658 | 35.675 | 2.955 | 1.00 | 16.46 | A | C | |
| ATOM | 1328 | C | AJA | A | 780 | 53.793 | 35.326 | 0.767 | 1.00 | 16.61 | A | C | |
| ATOM | 1329 | O | ALA | A | 780 | 53.822 | 36.274 | −0.015 | 1.00 | 16.59 | A | O | |
| ATOM | 1330 | N | ALA | A | 781 | 53.578 | 34.071 | 0.381 | 1.00 | 14.97 | A | N | |
| ATOM | 1331 | CA | ALA | A | 781 | 53.37 | 733.751 | −1.037 | 1.00 | 18.54 | A | C | |
| ATOM | 1332 | CB | ALA | A | 781 | 53.252 | 32.221 | −1.226 | 1.00 | 17.62 | A | C | |
| ATOM | 1333 | C | ALA | A | 781 | 52.155 | 34.464 | −1.623 | 1.00 | 20.68 | A | C | |
| ATOM | 1334 | O | ALA | A | 781 | 52.133 | 34.808 | −2.815 | 1.00 | 19.94 | A | O | |
| ATOM | 1335 | N | ARG | A | 782 | 51.135 | 34.680 | −0.794 | 1.00 | 12.58 | A | N | |
| ATOM | 1336 | CA | ARG | A | 782 | 49.934 | 35.354 | −1.250 | 1.00 | 19.87 | A | C | |
| ATOM | 1337 | CB | ARG | A | 782 | 48.877 | 35.390 | −0.140 | 1.00 | 17.69 | A | C | |
| ATOM | 1338 | CG | ARG | A | 782 | 49.346 | 36.096 | 1.115 | 1.00 | 22.13 | A | C | |
| ATOM | 1339 | CD | ARG | A | 782 | 48.245 | 36.267 | 2.143 | 1.00 | 18.87 | A | C | |
| ATOM | 1340 | NE | ARG | A | 782 | 48.815 | 36.800 | 3.380 | 1.00 | 16.17 | A | N | |
| ATOM | 1341 | CZ | ARG | A | 782 | 49.432 | 36.055 | 4.287 | 1.00 | 18.05 | A | C | |
| ATOM | 1342 | NH1 | ARG | A | 782 | 49.538 | 34.746 | 4.098 | 1.00 | 15.44 | A | N | |
| ATOM | 1343 | NH2 | ARG | A | 782 | 49.975 | 36.624 | 5.360 | 1.00 | 14.60 | A | N | |
| ATOM | 1344 | C | ARG | A | 782 | 50.259 | 36.774 | −1.697 | 1.00 | 18.63 | A | C | |
| ATOM | 1345 | O | ARG | A | 782 | 49.526 | 37.349 | −2.499 | 1.00 | 19.79 | A | O | |
| ATOM | 1346 | N | ASN | A | 783 | 51.355 | 37.337 | −1.180 | 1.00 | 16.35 | A | N | |
| ATOM | 1347 | CA | ASN | A | 783 | 51.754 | 38.695 | −1.538 | 1.00 | 21.66 | A | C | |
| ATOM | 1348 | CB | ASN | A | 783 | 52.066 | 39.526 | −0.286 | 1.00 | 17.66 | A | C | |
| ATOM | 1349 | CG | ASN | A | 783 | 50.825 | 39.861 | 0.510 | 1.00 | 19.83 | A | C | |
| ATOM | 1350 | OD1 | ASN | A | 783 | 49.801 | 40.231 | −0.062 | 1.00 | 21.64 | A | O | |
| ATOM | 1351 | ND2 | ASN | A | 783 | 50.909 | 39.744 | 1.836 | 1.00 | 15.81 | A | N | |
| ATOM | 1352 | C | ASN | A | 783 | 52.932 | 38.744 | −2.500 | 1.00 | 18.05 | A | C | |
| ATOM | 1353 | O | ASN | A | 783 | 53.789 | 39.629 | −2.422 | 1.00 | 18.47 | A | O | |
| ATOM | 1354 | N | VAL | A | 784 | 52.985 | 37.771 | −3.402 | 1.00 | 17.31 | A | N | |
| ATOM | 1355 | CA | VAL | A | 784 | 54.021 | 37.764 | 4.420 | 1.00 | 16.02 | A | C | |
| ATOM | 1356 | CB | VAL | A | 784 | 54.998 | 36.571 | −4.268 | 1.00 | 18.51 | A | C | |
| ATOM | 1357 | CG1 | VAL | A | 784 | 56.009 | 36.589 | −5.404 | 1.00 | 15.61 | A | C | |
| ATOM | 1358 | CG2 | VAL | A | 784 | 55.734 | 36.654 | −2.941 | 1.00 | 12.80 | A | C | |
| ATOM | 1359 | C | VAL | A | 784 | 53.238 | 37.626 | −5.723 | 1.00 | 16.94 | A | C | |
| ATOM | 1360 | O | VAL | A | 784 | 52.490 | 36.677 | −5.894 | 1.00 | 17.28 | A | O | |
| ATOM | 1361 | N | LEU | A | 785 | 53.386 | 38.589 | −6.625 | 1.00 | 19.95 | A | N | |
| ATOM | 1362 | CA | LEU | A | 785 | 52.668 | 38.538 | −7.894 | 1.00 | 22.85 | A | C | |
| ATOM | 1363 | CB | LEU | A | 785 | 52.051 | 39.909 | −8.199 | 1.00 | 24.49 | A | C | |
| ATOM | 1364 | CG | LEU | A | 785 | 50.659 | 40.206 | −7.622 | 1.00 | 34.20 | A | C | |
| ATOM | 1365 | CD1 | LEU | A | 785 | 50.517 | 39.642 | −6.232 | 1.00 | 27.64 | A | C | |
| ATOM | 1366 | CD2 | LEU | A | 785 | 50.419 | 41.704 | −7.624 | 1.00 | 18.93 | A | C | |
| ATOM | 1367 | C | LEU | A | 785 | 53.578 | 38.110 | −9.035 | 1.00 | 24.51 | A | C | |
| ATOM | 1368 | O | LEU | A | 785 | 54.789 | 38.305 | −8.980 | 1.00 | 22.25 | A | O | |
| ATOM | 1369 | N | LEU | A | 786 | 52.991 | 37.525 | −10.077 | 1.00 | 24.93 | A | N | |
| ATOM | 1370 | CA | LEU | A | 786 | 53.781 | 37.082 | −11.217 | 1.00 | 24.88 | A | C | |
| ATOM | 1371 | CB | LEU | A | 786 | 53.562 | 35.589 | −11.472 | 1.00 | 26.85 | A | C | |
| ATOM | 1372 | CG | LEU | A | 786 | 53.951 | 34.661 | −10.321 | 1.00 | 24.52 | A | C | |
| ATOM | 1373 | CD1 | LEU | A | 786 | 53.812 | 33.206 | −10.766 | 1.00 | 22.88 | A | C | |
| ATOM | 1374 | CD2 | LEU | A | 786 | 55.380 | 34.961 | −9.895 | 1.00 | 21.56 | A | C | |
| ATOM | 1375 | C | LEU | A | 786 | 53.406 | 37.879 | −12.455 | 1.00 | 24.87 | A | C | |
| ATOM | 1376 | O | LEU | A | 786 | 52.232 | 37.956 | −12.819 | 1.00 | 19.86 | A | O | |
| ATOM | 1377 | N | THR | A | 787 | 54.412 | 38.470 | −13.096 | 1.00 | 26.67 | A | N | |
| ATOM | 1378 | CA | THR | A | 787 | 54.181 | 39.275 | −14.291 | 1.00 | 31.86 | A | C | |
| ATOM | 1379 | CB | THR | A | 787 | 54.774 | 40.697 | −14.108 | 1.00 | 32.16 | A | C | |
| ATOM | 1380 | OG1 | THR | A | 787 | 54.289 | 41.556 | −15.145 | 1.00 | 35.95 | A | O | |
| ATOM | 1381 | CG2 | THR | A | 787 | 56.299 | 40.659 | −14.145 | 1.00 | 29.30 | A | C | |
| ATOM | 1382 | C | THR | A | 787 | 54.787 | 38.592 | −15.524 | 1.00 | 33.01 | A | C | |
| ATOM | 1383 | O | THR | A | 787 | 55.109 | 37.405 | −15.485 | 1.00 | 31.90 | A | O | |
| ATOM | 1384 | N | ASN | A | 788 | 54.934 | 39.333 | −16.615 | 1.00 | 36.22 | A | N | |
| ATOM | 1385 | CA | ASN | A | 788 | 55.486 | 38.774 | −17.846 | 1.00 | 40.57 | A | C | |
| ATOM | 1386 | CB | ASN | A | 788 | 55.762 | 39.896 | −18.844 | 1.00 | 42.89 | A | C | |
| ATOM | 1387 | CG | ASN | A | 788 | 54.527 | 40.706 | −19.149 | 1.00 | 42.43 | A | C | |
| ATOM | 1388 | OD1 | ASN | A | 788 | 53.576 | 40.206 | −19.749 | 1.00 | 43.79 | A | O | |
| ATOM | 1389 | ND2 | ASN | A | 788 | 54.527 | 41.963 | −18.726 | 1.00 | 43.62 | A | N | |
| ATOM | 1390 | C | ASN | A | 788 | 56.757 | 37.960 | −17.622 | 1.00 | 39.75 | A | C | |
| ATOM | 1391 | O | ASN | A | 788 | 57.674 | 38.394 | −16.925 | 1.00 | 44.43 | A | O | |
| ATOM | 1392 | N | GLY | A | 789 | 56.805 | 36.778 | −18.230 | 1.00 | 41.34 | A | N | |
| ATOM | 1393 | CA | GLY | A | 789 | 57.960 | 35.915 | −18.079 | 1.00 | 35.78 | A | C | |
| ATOM | 1394 | C | GLY | A | 789 | 57.871 | 35.197 | −16.750 | 1.00 | 36.30 | A | C | |
| ATOM | 1395 | O | GLY | A | 789 | 58.792 | 34.488 | −16.346 | 1.00 | 42.58 | A | O | |
| ATOM | 1396 | N | HIS | A | 790 | 56.738 | 35.383 | −16.080 | 1.00 | 34.24 | A | N | |
| ATOM | 1397 | CA | HIS | A | 790 | 56.492 | 34.792 | −14.772 | 1.00 | 36.84 | A | C | |
| ATOM | 1398 | CB | HIS | A | 790 | 56.488 | 33.266 | −14.877 | 1.00 | 33.92 | A | C | |
| ATOM | 1399 | CG | HIS | A | 790 | 55.255 | 32.723 | −15.530 | 1.00 | 39.92 | A | C | |
| ATOM | 1400 | CD2 | HIS | A | 790 | 54.989 | 32.424 | −16.824 | 1.00 | 35.95 | A | C | |
| ATOM | 1401 | ND1 | HIS | A | 790 | 54.081 | 32.509 | −14.839 | 1.00 | 38.27 | A | N | |
| ATOM | 1402 | CE1 | HIS | A | 790 | 53.145 | 32.103 | −15.679 | 1.00 | 35.69 | A | C | |
| ATOM | 1403 | NE2 | HIS | A | 790 | 53.670 | 32.044 | −16.889 | 1.00 | 36.41 | A | N | |
| ATOM | 1404 | C | HIS | A | 790 | 57.527 | 35.287 | −13.767 | 1.00 | 34.04 | A | C | |
| ATOM | 1405 | O | HIS | A | 790 | 57.957 | 34.557 | −12.868 | 1.00 | 39.85 | A | O | |
| ATOM | 1406 | N | VAL | A | 791 | 57.926 | 36.544 | −13.942 | 1.00 | 31.80 | A | N | |
| ATOM | 1407 | CA | VAL | A | 791 | 58.883 | 37.175 | −13.049 | 1.00 | 31.97 | A | C | |
| ATOM | 1408 | CB | VAL | A | 791 | 59.556 | 38.400 | −13.707 | 1.00 | 32.02 | A | C | |
| ATOM | 1409 | CG1 | VAL | A | 791 | 60.527 | 39.051 | −12.726 | 1.00 | 25.13 | A | C | |
| ATOM | 1410 | CG2 | VAL | A | 791 | 60.300 | 37.972 | −14.961 | 1.00 | 31.02 | A | C | |
| ATOM | 1411 | C | VAL | A | 791 | 58.102 | 37.627 | −11.818 | 1.00 | 27.71 | A | C | |
| ATOM | 1412 | O | VAL | A | 791 | 57.029 | 38.219 | −11.933 | 1.00 | 32.13 | A | O | |
| ATOM | 1413 | N | ALA | A | 792 | 58.646 | 37.339 | −10.642 | 1.00 | 28.53 | A | N | |
| ATOM | 1414 | CA | ALA | A | 792 | 57.996 | 37.692 | −9.389 | 1.00 | 24.21 | A | C | |
| ATOM | 1415 | CB | ALA | A | 792 | 58.495 | 36.773 | −8.281 | 1.00 | 23.90 | A | C | |
| ATOM | 1416 | C | ALA | A | 792 | 58.214 | 39.150 | −8.991 | 1.00 | 22.20 | A | C | |
| ATOM | 1417 | O | ALA | A | 792 | 59286 | 39.713 | −9.212 | 1.00 | 24.18 | A | O | |
| ATOM | 1418 | N | LYS | A | 793 | 57.187 | 39.749 | −8.399 | 1.00 | 19.99 | A | N | |
| ATOM | 1419 | CA | LYS | A | 793 | 57.248 | 41.131 | −7.932 | 1.00 | 20.74 | A | C | |
| ATOM | 1420 | CB | LYS | A | 793 | 56.574 | 42.081 | −8.932 | 1.00 | 25.52 | A | C | |
| ATOM | 1421 | CG | LYS | A | 793 | 53.389 | 42.424 | −10.178 | 1.00 | 21.48 | A | C | |
| ATOM | 1422 | CD | LYS | A | 793 | 56.555 | 43.295 | −11.121 | 1.00 | 24.82 | A | C | |
| ATOM | 1423 | CB | LYS | A | 793 | 57.371 | 43.802 | −12.302 | 1.00 | 25.83 | A | C | |
| ATOM | 1424 | NZ | LYS | A | 793 | 58.322 | 44.889 | −11.929 | 1.00 | 21.51 | A | N | |
| ATOM | 1425 | C | LYS | A | 793 | 56.533 | 41.258 | −6.580 | 1.00 | 19.38 | A | C | |
| ATOM | 1426 | O | LYS | A | 793 | 55.560 | 40.551 | −6.308 | 1.00 | 20.92 | A | O | |
| ATOM | 1427 | N | ILE | A | 794 | 57.008 | 42.168 | −5.741 | 1.00 | 21.18 | A | N | |
| ATOM | 1428 | CA | ILE | A | 794 | 56.370 | 42.379 | −4.452 | 1.00 | 22.72 | A | C | |
| ATOM | 1429 | CB | ILE | A | 794 | 57.321 | 42.032 | −3.294 | 1.00 | 25.75 | A | C | |
| ATOM | 1430 | CG2 | ILE | A | 794 | 57.605 | 40.525 | −3.304 | 1.00 | 26.33 | A | C | |
| ATOM | 1431 | CG1 | ILE | A | 794 | 58.610 | 42.863 | −3.404 | 1.00 | 27.20 | A | C | |
| ATOM | 1432 | CD1 | ILE | A | 794 | 59.584 | 42.688 | −2.243 | 1.00 | 23.60 | A | C | |
| ATOM | 1433 | C | ILE | A | 794 | 55.924 | 43.833 | −4.341 | 1.00 | 20.28 | A | C | |
| ATOM | 1434 | O | ILE | A | 794 | 56.489 | 44.715 | −4.982 | 1.00 | 23.20 | A | O | |
| ATOM | 1435 | N | ILE | A | 795 | 54.894 | 44.068 | −3.540 | 1.00 | 19.95 | A | N | |
| ATOM | 1436 | CA | GLY | A | 795 | 54.387 | 45.413 | −3.357 | 1.00 | 19.70 | A | C | |
| ATOM | 1437 | C | GLY | A | 795 | 53.482 | 45.440 | −2.147 | .00 | 22.98 | A | C | |
| ATOM | 1438 | O | GLY | A | 795 | 53.143 | 44.392 | −1.596 | 1.00 | 25.68 | A | O | |
| ATOM | 1439 | N | ASP | A | 796 | 53.107 | 46.636 | −1.713 | 1.00 | 22.67 | A | N | |
| ATOM | 1440 | CA | ASP | A | 796 | 52.213 | 46.769 | −0.576 | 1.00 | 26.29 | A | C | |
| ATOM | 1441 | CB | ASP | A | 796 | 52.286 | 48.179 | 0.004 | 1.00 | 23.94 | A | C | |
| ATOM | 1442 | CG | ASP | A | 796 | 51.588 | 48.281 | 1.336 | 1.00 | 29.84 | A | C | |
| ATOM | 1443 | OD1 | ASP | A | 796 | 50.543 | 47.619 | 1.485 | 1.00 | 30.78 | A | O | |
| ATOM | 1444 | OD2 | ASP | A | 796 | 52.075 | 49.013 | 2.222 | 1.00 | 24.56 | A | O | |
| ATOM | 1445 | C | ASP | A | 796 | 50.805 | 46.509 | −1.113 | 1.00 | 26.30 | A | C | |
| ATOM | 1446 | O | ASP | A | 796 | 50.269 | 47.320 | −1.869 | 1.00 | 24.33 | A | O | |
| ATOM | 1447 | N | PHE | A | 797 | 50.217 | 45.377 | −0.741 | 1.00 | 24.34 | A | N | |
| ATOM | 1448 | CA | PHE | A | 797 | 48.882 | 45.027 | −1.223 | 1.00 | 29.06 | A | C | |
| ATOM | 1449 | CB | PHE | A | 797 | 48.892 | 43.627 | −1.840 | 1.00 | 21.19 | A | C | |
| ATOM | 1450 | CG | PHE | A | 797 | 49.958 | 43.419 | −2.868 | 1.00 | 23.96 | A | C | |
| ATOM | 1451 | CD1 | PHE | A | 797 | 50.698 | 42.243 | −2.883 | 1.00 | 18.21 | A | C | |
| ATOM | 1452 | CD2 | PHE | A | 797 | 50.219 | 44.386 | −3.825 | 1.00 | 17.68 | A | C | |
| ATOM | 1453 | CE1 | PHE | A | 797 | 51.678 | 42.034 | −3.830 | 1.00 | 19.92 | A | C | |
| ATOM | 1454 | CE2 | PHE | A | 797 | 51.204 | 44.183 | −4.782 | 1.00 | 19.67 | A | C | |
| ATOM | 1455 | CZ | PHE | A | 797 | 51.931 | 43.007 | −4.783 | 1.00 | 21.31 | A | C | |
| ATOM | 1456 | C | PHE | A | 797 | 47.815 | 45.062 | −0.133 | 1.00 | 27.83 | A | C | |
| ATOM | 1457 | O | PHE | A | 797 | 46.676 | 44.646 | −0.367 | 1.00 | 28.25 | A | O | |
| ATOM | 1458 | N | GLY | A | 798 | 48.174 | 45.559 | 1.047 | 1.00 | 24.16 | A | N | |
| ATOM | 1459 | CA | GLY | A | 798 | 47.226 | 45.606 | 2.151 | 1.00 | 28.22 | A | C | |
| ATOM | 1460 | C | GLY | A | 798 | 45.846 | 46.190 | 1.866 | 1.00 | 35.83 | A | C | |
| ATOM | 1461 | O | GLY | A | 798 | 44.822 | 45.557 | 2.148 | 1.00 | 35.61 | A | O | |
| ATOM | 1462 | N | LEU | A | 799 | 45.812 | 47.398 | 1.309 | 1.00 | 34.09 | A | N | |
| ATOM | 1463 | CA | LEU | A | 799 | 44.553 | 48.075 | 1.00 | 41.00 | 35.90 | A | C | |
| ATOM | 1464 | CB | LEU | A | 799 | 44.844 | 49.409 | 0.304 | 1.00 | 40.02 | A | C | |
| ATOM | 1465 | CG | LEU | A | 799 | 43.672 | 50.324 | −0.078 | 1.00 | 48.48 | A | C | |
| ATOM | 1466 | CD1 | LEU | A | 799 | 43.052 | 49.877 | −1.389 | 1.00 | 49.24 | A | C | |
| ATOM | 1467 | CD2 | LEU | A | 799 | 42.650 | 50.333 | 1.047 | 1.00 | 47.29 | A | C | |
| ATOM | 1468 | C | LEU | A | 799 | 43.581 | 47.244 | 0.162 | 1.00 | 33.17 | A | C | |
| ATOM | 1469 | O | LEU | A | 799 | 42.368 | 47.434 | 0.242 | 1.00 | 35.80 | A | O | |
| ATOM | 1470 | N | ALA | A | 800 | 44.110 | 46.338 | −0.652 | 1.00 | 27.34 | A | N | |
| ATOM | 1471 | CA | ALA | A | 800 | 43.272 | 45.481 | −1.489 | 1.00 | 32.20 | A | C | |
| ATOM | 1472 | CB | ALA | A | 800 | 43.854 | 45.401 | −2.895 | 1.00 | 28.94 | A | C | |
| ATOM | 1473 | C | ALA | A | 800 | 43.151 | 44.075 | −0.886 | 1.00 | 29.06 | A | C | |
| ATOM | 1474 | O | ALA | A | 800 | 42.590 | 43.170 | −1.503 | 1.00 | 32.92 | A | O | |
| ATOM | 1475 | N | ARG | A | 801 | 43.682 | 43.907 | 0.322 | 1.00 | 28.46 | A | N | |
| ATOM | 1476 | CA | ARG | A | 801 | 43.654 | 42.631 | 1.022 | 1.00 | 28.47 | A | C | |
| ATOM | 1477 | CB | ARG | A | 801 | 44.954 | 42.466 | 1.821 | 1.00 | 35.65 | A | C | |
| ATOM | 1478 | CG | ARG | A | 801 | 45.104 | 41.155 | 2.590 | 1.00 | 42.08 | A | C | |
| ATOM | 1479 | CD | ARG | A | 801 | 45.118 | 39.932 | 1.683 | 1.00 | 39.14 | A | C | |
| ATOM | 1480 | NE | ARG | A | 801 | 46.309 | 39.827 | 0.838 | 1.00 | 33.07 | A | N | |
| ATOM | 1481 | CZ | ARG | A | 801 | 46.470 | 38.877 | −0.079 | 1.00 | 24.03 | A | C | |
| ATOM | 1482 | NH1 | ARG | A | 801 | 45.523 | 37.967 | −0.257 | 1.00 | 29.78 | A | N | |
| ATOM | 1483 | NH2 | ARG | A | 801 | 47.558 | 38.841 | −0.831 | 1.00 | 20.54 | A | N | |
| ATOM | 1484 | C | ARG | A | 801 | 42.434 | 42.535 | 1.951 | 1.00 | 33.90 | A | C | |
| ATOM | 1485 | O | ARG | A | 801 | 42.241 | 43.367 | 2.839 | 1.00 | 26.76 | A | O | |
| ATOM | 1486 | N | ASP | A | 802 | 41.609 | 41.517 | 1.726 | 1.00 | 28.62 | A | N | |
| ATOM | 1487 | CA | ASP | A | 802 | 40.409 | 41.285 | 2.529 | 1.00 | 25.00 | A | C | |
| ATOM | 1488 | CB | ASP | A | 802 | 39.469 | 40.365 | 1.739 | 1.00 | 29.05 | A | C | |
| ATOM | 1489 | CG | ASP | A | 802 | 38.216 | 39.987 | 2.501 | 1.00 | 32.45 | A | C | |
| ATOM | 1490 | OD1 | ASP | A | 802 | 37.783 | 40.754 | 3.389 | 1.00 | 27.77 | A | O | |
| ATOM | 1491 | OD2 | ASP | A | 802 | 37.654 | 38.914 | 2.184 | 1.00 | 33.83 | A | O | |
| ATOM | 1492 | C | ASP | A | 802 | 40.842 | 40.648 | 3.855 | 1.00 | 23.80 | A | C | |
| ATOM | 1493 | O | ASP | A | 802 | 40.477 | 39.517 | 4.168 | 1.00 | 23.73 | A | O | |
| ATOM | 1494 | N | ILE | A | 803 | 41.623 | 41.395 | 4.631 | 1.00 | 24.15 | A | N | |
| ATOM | 1495 | CA | ILE | A | 803 | 42.157 | 40.917 | 5.908 | 1.00 | 25.86 | A | C | |
| ATOM | 1496 | CB | ILE | A | 803 | 43.110 | 41.965 | 6.525 | 1.00 | 25.35 | A | C | |
| ATOM | 1497 | CG2 | ILE | A | 803 | 43.393 | 41.627 | 7.964 | 1.00 | 33.76 | A | C | |
| ATOM | 1498 | CG1 | ILE | A | 803 | 44.418 | 42.006 | 5.731 | 1.00 | 30.72 | A | C | |
| ATOM | 1499 | CD1 | ILE | A | 803 | 45.442 | 42.960 | 6.301 | 1.00 | 39.69 | A | C | |
| ATOM | 1500 | C | ILE | A | 803 | 41.161 | 40.494 | 6.986 | 1.00 | 23.68 | A | C | |
| ATOM | 1501 | O | ILE | A | 803 | 41.412 | 39.535 | 7.724 | 1.00 | 25.42 | A | O | |
| ATOM | 1502 | N | MET | A | 804 | 40.045 | 41.205 | 7.088 | 1.00 | 22.39 | A | N | |
| ATOM | 1503 | CA | MET | A | 804 | 39.032 | 40.893 | 8.096 | 1.00 | 25.91 | A | C | |
| ATOM | 1504 | CB | MET | A | 804 | 37.892 | 41.916 | 8.031 | 1.00 | 27.18 | A | C | |
| ATOM | 1505 | CG | MET | A | 804 | 38.331 | 43.359 | 8.194 | 1.00 | 34.73 | A | C | |
| ATOM | 1506 | SD | MET | A | 804 | 39.110 | 43.639 | 9.793 | 1.00 | 34.84 | A | S | |
| ATOM | 1507 | CE | MET | A | 804 | 37.688 | 43.389 | 10.885 | 1.00 | 38.16 | A | C | |
| ATOM | 1508 | C | MET | A | 804 | 38.438 | 39.494 | 7.939 | 1.00 | 24.53 | A | C | |
| ATOM | 1509 | O | MET | A | 804 | 38.072 | 38.858 | 8.923 | 1.00 | 24.91 | A | O | |
| ATOM | 1510 | N | ASN | A | 805 | 38.354 | 39.013 | 6.702 | 1.00 | 19.70 | A | N | |
| ATOM | 1511 | CA | ASN | A | 805 | 37.757 | 37.714 | 6.436 | 1.00 | 20.76 | A | C | |
| ATOM | 1512 | CB | ASN | A | 805 | 36.661 | 37.884 | 5.378 | 1.00 | 25.59 | A | C | |
| ATOM | 1513 | CG | ASN | A | 805 | 35.599 | 38.877 | 5.803 | 1.00 | 21.78 | A | C | |
| ATOM | 1514 | OD1 | ASN | A | 805 | 35.017 | 38.747 | 6.875 | 1.00 | 24.02 | A | O | |
| ATOM | 1515 | ND2 | ASN | A | 805 | 35.343 | 39.877 | 4.968 | 1.00 | 23.80 | A | N | |
| ATOM | 1516 | C | ASN | A | 805 | 38.736 | 36.621 | 6.010 | 1.00 | 28.45 | A | C | |
| ATOM | 1517 | O | ASN | A | 805 | 38.331 | 35.586 | 5.475 | 1.00 | 25.85 | A | O | |
| ATOM | 1518 | N | ASP | A | 806 | 40.021 | 36.854 | 6.251 | 1.00 | 23.59 | A | N | |
| ATOM | 1519 | CA | ASP | A | 806 | 41.061 | 35.896 | 5.906 | 1.00 | 20.85 | A | C | |
| ATOM | 1520 | CB | ASP | A | 806 | 42.189 | 36.606 | 5.154 | 1.00 | 25.77 | A | C | |
| ATOM | 1521 | CC | ASP | A | 806; | 43.250 | 35.654 | 4.651 | 1.00 | 20.88 | A | C | |
| ATOM | 1522 | OD1 | ASP | A | 806 | 43.532 | 34.646 | 5.335 | 1.00 | 22.17 | A | O | |
| ATOM | 1523 | OD2 | ASP | A | 806 | 43.813 | 35.935 | 3.576 | 1.00 | 25.61 | A | O | |
| ATOM | 1524 | C | ASP | A | 806 | 41.585 | 35.327 | 7.216 | 1.00 | 20.23 | A | C | |
| ATOM | 1525 | O | ASP | A | 806 | 42.265 | 36.026 | 7.972 | 1.00 | 23.86 | A | O | |
| ATOM | 1526 | N | SER | A | 807 | 41.278 | 34.063 | 7.485 | 1.00 | 21.93 | A | N | |
| ATOM | 1527 | CA | SER | A | 807 | 41.701 | 33.426 | 8.734 | 1.00 | 26..38 | A | C | |
| ATOM | 1528 | CB | SER | A | 807 | 41.081 | 32.034 | 8.853 | 1.00 | 21.67 | A | C | |
| ATOM | 1529 | CG | SER | A | 807 | 41.378 | 31.270 | 7.709 | 1.00 | 26.13 | A | O | |
| ATOM | 1530 | C | SER | A | 807 | 43.210 | 33.333 | 8.935 | 1.00 | 25.96 | A | C | |
| ATOM | 1531 | O | SER | A | 807 | 43.672 | 32.919 | 10.000 | 1.00 | 28.13 | A | O | |
| ATOM | 1532 | N | ASN | A | 808 | 43.976 | 33.707 | 7.915 | 1.00 | 22.59 | A | N | |
| ATOM | 1533 | CA | ASN | A | 808 | 45.420 | 33692 | 8.026 | 1.00 | 24.20 | A | C | |
| ATOM | 1534 | CB | ASN | A | 808 | 46.067 | 33.734 | 6.636 | 1.00 | 26.55 | A | C | |
| ATOM | 1535 | CG | ASN | A | 808 | 45.968 | 32.399 | 5.905 | 1.00 | 30.23 | A | C | |
| ATOM | 1536 | OD1 | ASN | A | 808 | 45.640 | 32.343 | 4.718 | 1.00 | 25.73 | A | O | |
| ATOM | 1537 | ND2 | ASN | A | 808 | 46.262 | 31.323 | 6.611 | 1.00 | 23.16 | A | N | |
| ATOM | 1538 | C | ASN | A | 808 | 45.840 | 34.907 | 8.849 | 1.00 | 2.6.53 | A | C | |
| ATOM | 1539 | O | ASN | A | 808 | 46.986 | 35.002 | 9.287 | 1.00 | 29.13 | A | O | |
| ATOM | 1540 | N | TYR | A | 809 | 44.907 | 35.840 | 9.050 | 1.00 | 19.60 | A | N | |
| ATOM | 1541 | CA | TYR | A | 809 | 45.194 | 37.026 | 9.849 | 1.00 | 16.84 | A | C | |
| ATOM | 1542 | CB | TYR | A | 809 | 44.773 | 38.303 | 9.122 | 1.00 | 20.05 | A | C | |
| ATOM | 1543 | CG | TYR | A | 809 | 45.607 | 38.583 | 7.893 | 1.00 | 19.88 | A | C | |
| ATOM | 1544 | CD1 | TYR | A | 809 | 45.343 | 37.938 | 6.696 | 1.00 | 18.75 | A | C | |
| ATOM | 1545 | CE1 | TYR | A | 809 | 46.129 | 38.160 | 5.571 | 1.00 | 15.18 | A | C | |
| ATOM | 1546 | CD2 | TYR | A | 809 | 46.687 | 39.465 | 7.943 | 1.00 | 12.79 | A | C | |
| ATOM | 1547 | CE2 | TYR | A | 809 | 47.476 | 39.699 | 6.823 | 1.00 | 16.10 | A | C | |
| ATOM | 1548 | CZ | TYR | A | 809 | 47.189 | 39.042 | 5.638 | 1.00 | 13.32 | A | C | |
| ATOM | 1549 | OH | TYR | A | 809 | 47.955 | 39.258 | 4.512 | 1.00 | 16.28 | A | O | |
| ATOM | 1556 | C | TYR | A | 809 | 44.466 | 36.904 | 11.180 | 1.00 | 20.83 | A | C | |
| ATOM | 1551 | O | TYR | A | 809 | 43.246 | 37.056 | 11.259 | 1.00 | 21.15 | A | O | |
| ATOM | 1552 | N | ILE | A | 810 | 45.247 | 36.621 | 12.218 | 1.00 | 29.90 | A | N | |
| ATOM | 1553 | CA | ILE | A | 810 | 44.759 | 36.424 | 13.575 | 1.00 | 32.50 | A | C | |
| ATOM | 1554 | CE | ILE | A | 810 | 45.804 | 35.650 | 14.405 | 1.00 | 35.70 | A | C | |
| ATOM | 1555 | CG2 | ILE | A | 810 | 45.311 | 35.450 | 15.826 | 1.00 | 34.48 | A | C | |
| ATOM | 1556 | CG1 | ILE | A | 810 | 46.107 | 34.312 | 13.728 | 1.00 | 35.73 | A | C | |
| ATOM | 1557 | CD1 | ILE | A | 810 | 44.888 | 33.459 | 13.488 | 1.00 | 41.45 | A | C | |
| ATOM | 1558 | C | ILE | A | 810 | 44.425 | 37.717 | 14.304 | 1.00 | 35.12 | A | C | |
| ATOM | 1559 | O | ILE | A | 810 | 45.158 | 38.702 | 14.227 | 1.00 | 32.73 | A | O | |
| ATOM | 1560 | N | VAL | A | 811 | 43.316 | 37.697 | 15.031 | 1.00 | 35.36 | A | N | |
| ATOM | 1561 | CA | VAL | A | 811 | 42.876 | 38.863 | 15.778 | 1.00 | 40.37 | A | C | |
| ATOM | 1562 | CE | VAL | A | 811 | 41.413 | 38.706 | 16.247 | 1.00 | 39.63 | A | C | |
| ATOM | 1563 | CG1 | VAL | A | 811 | 40.916 | 40.008 | 16.847 | 1.00 | 41.96 | A | C | |
| ATOM | 1564 | CG2 | VAL | A | 811 | 40.534 | 38.283 | 15.084 | 1.00 | 40.81 | A | C | |
| ATOM | 1565 | C | VAL | A | 811 | 43.753 | 39.067 | 17.007 | 1.00 | 43.36 | A | C | |
| ATOM | 1566 | O | VAL | A | 811 | 43.971 | 38.141 | 17.788 | 1.00 | 45.36 | A | O | |
| ATOM | 1567 | N | LYS | A | 812 | 44.271 | 40.279 | 17.161 | 1.00 | 48.53 | A | N | |
| ATOM | 1568 | CA | LYS | A | 812 | 45.098 | 40.619 | 18.312 | 1.00 | 53.89 | A | C | |
| ATOM | 1569 | CE | LYS | A | 812 | 46.539 | 40.904 | 17.892 | 1.00 | 57.97 | A | C | |
| ATOM | 1570 | CG | LYS | A | 812 | 47.449 | 41.242 | 19.064 | 1.00 | 61.64 | A | C | |
| ATOM | 1571 | CD | LYS | A | 812 | 48.792 | 41.772 | 18.602 | 1.00 | 65.44 | A | C | |
| ATOM | 1572 | CE | LYS | A | 812 | 49.650 | 42.189 | 19.788 | 1.00 | 64.89 | A | C | |
| ATOM | 1573 | NZ | LYS | A | 812 | 50.974 | 42.717 | 19.360 | 1.00 | 59.14 | A | N | |
| ATOM | 1574 | C | LYS | A | 812 | 44.487 | 41.871 | 18.929 | 1.00 | 58.46 | A | C | |
| ATOM | 1575 | O | LYS | A | 812 | 45.171 | 42.871 | 19.156 | 1.00 | 61.10 | A | O | |
| ATOM | 1576 | N | GLY | A | 813 | 43.183 | 41.806 | 19.183 | 1.00 | 58.36 | A | N | |
| ATOM | 1577 | CA | GLY | A | 813 | 42.483 | 42.936 | 19.758 | 1.00 | 55.86 | A | C | |
| ATOM | 1578 | C | GLY | A | 813 | 42.277 | 44.061 | 18.760 | 1.00 | 58.07 | A | C | |
| ATOM | 1579 | O | GLY | A | 813 | 41.268 | 44.109 | 18.054 | 1.00 | 52.41 | A | O | |
| ATOM | 1580 | N | ASN | A | 814 | 43.254 | 44.959 | 18.692 | 1.00 | 62.47 | A | N | |
| ATOM | 1581 | CA | ASN | A | 814 | 43.193 | 46.113 | 17.801 | 1.00 | 65.67 | A | C | |
| ATOM | 1582 | CE | ASN | A | 814 | 43.982 | 47.270 | 18.414 | 1.00 | 68.89 | A | C | |
| ATOM | 1583 | CG | ASN | A | 814 | 45.456 | 46.945 | 18.573 | 1.00 | 71.72 | A | C | |
| ATOM | 1584 | OD1 | ASN | A | 814 | 45.820 | 45.950 | 19.203 | 1.00 | 73.23 | A | O | |
| ATOM | 1585 | ND2 | ASN | A | 814 | 46.314 | 47.784 | 18.000 | 1.00 | 72.88 | A | N | |
| ATOM | 1586 | C | ASN | A | 814 | 43.719 | 45.854 | 16.392 | 1.00 | 65.63 | A | C | |
| ATOM | 1587 | O | ASN | A | 814 | 43.876 | 46.793 | 15.609 | 1.00 | 67.90 | A | O | |
| ATOM | 1588 | N | ALA | A | 815 | 43.991 | 44.598 | 16.054 | 1.00 | 60.23 | A | N | |
| ATOM | 1589 | CA | ALA | A | 815 | 44.516 | 44.314 | 14.726 | 1.00 | 52.04 | A | C | |
| ATOM | 1590 | CB | ALA | A | 815 | 45.993 | 44.690 | 14.676 | 1.00 | 52.74 | A | C | |
| ATOM | 1591 | C | ALA | A | 815 | 44.340 | 42.878 | 14.254 | 1.00 | 46.58 | A | C | |
| ATOM | 1592 | O | ALA | A | 815 | 43.857 | 42.016 | 14.984 | 1.00 | 45.52 | A | O | |
| ATOM | 1593 | N | ARG | A | 816 | 44.739 | 42.649 | 13.009 | 1.00 | 40.22 | A | N | |
| ATOM | 1594 | CA | ARG | A | 816 | 44.683 | 41.341 | 12.368 | 1.00 | 34.96 | A | C | |
| ATOM | 1595 | CE | ARG | A | 816 | 43.739 | 41.368 | 11.168 | 1.00 | 40.08 | A | C | |
| ATOM | 1596 | CG | ARG | A | 816 | 42.434 | 40.629 | 11.355 | 1.00 | 52.11 | A | C | |
| ATOM | 1597 | CD | ARG | A | 816 | 41.455 | 41.411 | 12.194 | 1.00 | 49.21 | A | C | |
| ATOM | 1598 | NE | ARG | A | 816 | 40.168 | 40.731 | 12.263 | 1.00 | 55.65 | A | N | |
| ATOM | 1599 | CZ | ARG | A | 816 | 39.101 | 41.225 | 12.876 | 1.00 | 61.09 | A | C | |
| ATOM | 1600 | NH1 | ARG | A | 816 | 39.172 | 42.408 | 13.473 | 1.00 | 67.17 | A | N | |
| ATOM | 1601 | NH2 | ARG | A | 816 | 37.966 | 40.543 | 12.888 | 1.00 | 64.77 | A | N | |
| ATOM | 1602 | C | ARG | A | 816 | 46.100 | 41.106 | 11.871 | 1.00 | 26.90 | A | C | |
| ATOM | 1603 | O | ARG | A | 816 | 46.578 | 41.837 | 11.004 | 1.00 | 25.06 | A | O | |
| ATOM | 1604 | N | LEU | A | 817 | 46.773 | 40.093 | 12.401 | 1.00 | 23.26 | A | N | |
| ATOM | 1605 | CA | LEU | A | 817 | 48.147 | 39.841 | 11.987 | 1.00 | 26.71 | A | C | |
| ATOM | 1606 | CE | LEU | A | 817 | 49.083 | 40.064 | 13.178 | 1.00 | 34.47 | A | C | |
| ATOM | 1607 | CG | LEU | A | 817 | 48.964 | 41.429 | 13.869 | 1.00 | 38.57 | A | C | |
| ATOM | 1608 | CD1 | LEU | A | 817 | 49869 | 41.454 | 15.088 | 1.00 | 41.68 | A | C | |
| ATOM | 1609 | CD2 | LEU | A | 817 | 49.341 | 42.543 | 12.897 | 1.00 | 38.27 | A | C | |
| ATOM | 1610 | C | LEU | A | 817 | 48.388 | 38.452 | 11.403 | 1.00 | 23.81 | A | C | |
| ATOM | 1611 | O | LEU | A | 817 | 47.781 | 37.471 | 11.826 | 1.00 | 24.13 | A | O | |
| ATOM | 1612 | N | PRO | A | 818 | 49.290 | 38.357 | 10.413 | 1.00 | 22.29 | A | N | |
| ATOM | 1613 | CD | PRO | A | 818 | 50.001 | 39.483 | 9.775 | 1.00 | 20.86 | A | C | |
| ATOM | 1614 | CA | PRO | A | 818 | 49.628 | 37.088 | 9.763 | 1.00 | 19.06 | A | C | |
| ATOM | 1615 | CB | PRO | A | 818 | 50.242 | 37.541 | 8.446 | 1.00 | 22.27 | A | C | |
| ATOM | 1616 | CG | PRO | A | 818 | 51.012 | 38.763 | 8.872 | 1.00 | 27.08 | A | C | |
| ATOM | 1617 | C | PRO | A | 818 | 50.632 | 36.350 | 10.649 | 1.00 | 21.75 | A | C | |
| ATOM | 1618 | O | PRO | A | 818 | 51.782 | 36.156 | 10.269 | 1.00 | 23.01 | A | O | |
| ATOM | 1619 | N | VAL | A | 819 | 50.183 | 35.958 | 11.837 | 1.00 | 16.86 | A | N | |
| ATOM | 1620 | CA | VAL | A | 819 | 51.030 | 35.281 | 12.818 | 1.00 | 20.67 | A | C | |
| ATOM | 1621 | CB | VAL | A | 819 | 50.179 | 34.763 | 14.003 | 1.00 | 22.75 | A | C | |
| ATOM | 1622 | CG1 | VAL | A | 819 | 51.059 | 34.006 | 14.986 | 1.00 | 24.72 | A | C | |
| ATOM | 1623 | CG2 | VAL | A | 819 | 49.495 | 35.938 | 14.702 | 1.00 | 25.28 | A | C | |
| ATOM | 1624 | C | VAL | A | 819 | 51.939 | 34.139 | 12.35.6 | 1.00 | 23.31 | A | C | |
| ATOM | 1625 | O | VAL | A | 819 | 531096 | 34.074 | 12.768 | 1.00 | 21.31 | A | O | |
| ATOM | 1626 | N | LYS | A | 820 | 51.433 | 33.234 | 11.519 | 1.00 | 20.99 | A | N | |
| ATOM | 1627 | CA | LYS | A | 820 | 52.251 | 32.106 | 11.072 | 1.00 | 23.20 | A | C | |
| ATOM | 1628 | CB | LYS | A | 820 | 51.402 | 31.111 | 10.275 | 1.00 | 22.69 | A | C | |
| ATOM | 1629 | CG | LYS | A | 820 | 50.436 | 30.326 | 11.132 | 1.00 | 18.71 | A | C | |
| ATOM | 1630 | CD | LYS | A | 820 | 49.887 | 29.114 | 10.400 | 1.00 | 19.77 | A | C | |
| ATOM | 1631 | CE | LYS | A | 820 | 49.054 | 28.248 | 11.334 | 1.00 | 28.95 | A | C | |
| ATOM | 1632 | NZ | LYS | A | 820 | 48.776 | 26.897 | 10.757 | 1.00 | 35.62 | A | N | |
| ATOM | 1633 | C | LYS | A | 820 | 53.473 | 32.504 | 10.249 | 1.00 | 21.07 | A | C | |
| ATOM | 1634 | O | LYS | A | 820 | 54.395 | 31.708 | 10.073 | 1.00 | 18.03 | A | O | |
| ATOM | 1635 | N | TRP | A | 821 | 53.470 | 33.736 | 9.751 | 1.00 | 21.40 | A | N | |
| ATOM | 1636 | CA | TRP | A | 821 | 54.564 | 34.262 | 8.938 | 1.00 | 18.27 | A | C | |
| ATOM | 1637 | CB | TRP | A | 821 | 53.997 | 35.041 | 7.750 | 1.00 | 18.88 | A | C | |
| ATOM | 1638 | CG | TRP | A | 82.1 | 53.492 | 34.162 | 6.661 | 1.00 | 17.63 | A | C | |
| ATOM | 1639 | CD2 | TRP | A | 821 | 52.204 | 33.534 | 6.593 | 1.00 | 11.44 | A | C | |
| ATOM | 1640 | CE2 | TRP | A | 821 | 52.187 | 32.742 | 5.419 | 1.00 | 12.15 | A | C | |
| ATOM | 1641 | CE3 | TRP | A | 821 | 51.063 | 33.562 | 7.408 | 1.00 | 10.74 | A | C | |
| ATOM | 1642 | CD1 | TRP | A | 821 | 54.185 | 33.743 | 5.556 | 1.00 | 14.77 | A | C | |
| ATOM | 1643 | NE1 | TRP | A | 821 | 53.405 | 32.888 | 4.806 | 1.00 | 15.37 | A | N | |
| ATOM | 1644 | CZ2 | TRP | A | 821 | 51.078 | 31.980 | 5.042 | 1.00 | 11.08 | A | C | |
| ATOM | 1645 | CZ3 | TRP | A | 821 | 49.953 | 32.804 | 7.034 | 1.130 | 14.97 | A | C | |
| ATOM | 1646 | CH2 | TRP | A | 821 | 49.970 | 32.019 | 5.857 | 1.00 | 13.23 | A | C | |
| ATOM | 1647 | C | TRP | A | 821 | 55.482 | 35.195 | 9.724 | 1.00 | 24.23 | A | C | |
| ATOM | 1648 | O | TRP | A | 821 | 56.555 | 35.559 | 9.238 | 1.00 | 20.66 | A | O | |
| ATOM | 1649 | N | MET | A | 822 | 55.064 | 35.559 | 10.936 | 1.00 | 19.97 | A | N | |
| ATOM | 1650 | CA | MET | A | 822 | 55.809 | 36.506 | 11.776 | 1.00 | 22.80 | A | C | |
| ATOM | 1651 | CB | MET | A | 822 | 54.818 | 37.297 | 12.635 | 1.00 | 21.76 | A | C | |
| ATOM | 1652 | CG | MET | A | 822 | 53.912 | 38.219 | 11.831 | 1.00 | 22.97 | A | C | |
| ATOM | 1653 | SD | MET | A | 822 | 52.525 | 38.869 | 12.789 | 1.00 | 26.94 | A | 5 | |
| ATOM | 1654 | CB | MET | A | 822 | 53.420 | 39.708 | 14.129 | 1.00 | 24.56 | A | C | |
| ATOM | 1655 | C | MET | A | 822 | 56.931 | 35.988 | 12.677 | 1.00 | 21.20 | A | C | |
| ATOM | 1656 | O | MET | A | 822 | 56.814 | 34.930 | 13.298 | 1.00 | 21.09 | A | O | |
| ATOM | 1657 | N | ALA | A | 823 | 58.019 | 36.760 | 12.7.35 | 1.00 | 20.51 | A | N | |
| ATOM | 1658 | CA | ALA | A | 823 | 59.173 | 36.446 | 13.571 | 1.00 | 24.19 | A | C | |
| ATOM | 1659 | CB | ALA | A | 823 | 60.298 | 37.440 | 13.306 | 1.00 | 28.32 | A | C | |
| ATOM | 1660 | C | ALA | A | 823 | 58.716 | 36.558 | 15.022 | 1.00 | 22.49 | A | C | |
| ATOM | 1661 | O | ALA | A | 823 | 57.810 | 37.318 | 15.333 | 1.00 | 23.60 | A | O | |
| ATOM | 1662 | N | PRO | A | 824 | 59.351 | 35.807 | 15.931 | 1.00 | 28.05 | A | N | |
| ATOM | 1663 | CD | PRO | A | 824 | 60.548 | 34.969 | 15.747 | 1.00 | 30.69 | A | C | |
| ATOM | 1664 | CA | PRO | A | 824 | 58.961 | 35.860 | 17.341 | 1.00 | 26.19 | A | C | |
| ATOM | 1665 | CB | PRO | A | 824 | 59.937 | 34.891 | 18.005 | 1.00 | 25.24 | A | C | |
| ATOM | 1666 | CG | PRO | A | 824 | 61.147 | 34.969 | 17.131 | 1.00 | 30.82 | A | C | |
| ATOM | 1667 | C | PRO | A | 824 | 58.984 | 37.255 | 17.970 | 1.00 | 29.13 | A | C | |
| ATOM | 1668 | O | PRO | A | 824 | 58.041 | 37.639 | 18.659 | 1.00 | 35.79 | A | O | |
| ATOM | 1669 | N | GLU | A | 825 | 60.042 | 38.021 | 17.735 | 1.00 | 27.58 | A | N | |
| ATOM | 1670 | CA | GLU | A | 825 | 60.108 | 39.354 | 18.325 | 1.00 | 31.40 | A | C | |
| ATOM | 1671 | CB | GLU | A | 825 | 61.429 | 40.052 | 17.980 | 1.00 | 28.59 | A | C | |
| ATOM | 1672 | CG | GLU | A | 825 | 61.567 | 40.452 | 16.525 | 1.00 | 29.84 | A | C | |
| ATOM | 1673 | CD | GLU | A | 825 | 62.165 | 39.349 | 15.676 | 1.00 | 32.35 | A | C | |
| ATOM | 1674 | OE1 | GLU | A | 825 | 62.117 | 38.173 | 16.096 | 1.00 | 28.13 | A | O | |
| ATOM | 1675 | OE2 | GLU | A | 825 | 62.680 | 39.664 | 14.587 | 1.00 | 23.74 | A | O | |
| ATOM | 1676 | C | GLU | A | 825 | 58.944 | 40.237 | 17.885 | 1.00 | 33.54 | A | C | |
| ATOM | 1677 | O | GLU | A | 825 | 58.487 | 41.076 | 18.654 | 1.00 | 35.36 | A | O | |
| ATOM | 1678 | N | SER | A | 826 | 58.459 | 40.054 | 16.656 | 1.00 | 29.58 | A | N | |
| ATOM | 1679 | CA | SER | A | 826 | 57.349 | 40.868 | 16.162 | 1.00 | 27.62 | A | C | |
| ATOM | 1680 | CB | SER | A | 826 | 57.178 | 40.696 | 14.649 | 1.00 | 22.84 | A | C | |
| ATOM | 1681 | CG | SER | A | 826 | 58.327 | 41.142 | 13.950 | 1.00 | 28.90 | A | O | |
| ATOM | 1682 | C | SER | A | 826 | 56.055 | 40.493 | 16.869 | 1.00 | 29.24 | A | C | |
| ATOM | 1683 | 0 | SER | A | 826 | 55.243 | 41.358 | 17.206 | 1.00 | 28.92 | A | O | |
| ATOM | 1684 | N | ILE | A | 827 | 55.872 | 39.194 | 17.085 | 1.00 | 26.07 | A | N | |
| ATOM | 1685 | CA | ILE | A | 827 | 54.692 | 38.682 | 17.760 | 1.00 | 30.31 | A | C | |
| ATOM | 1686 | CB | ILE | A | 827 | 54.590 | 37.152 | 17.617 | 1.00 | 32.61 | A | C | |
| ATOM | 1687 | CG2 | ILE | A | 827 | 53.456 | 36.630 | 18.476 | 1.00 | 32.96 | A | C | |
| ATOM | 1688 | CG1 | ILE | A | 827 | 54.379 | 36.769 | 16.151 | 1.00 | 29.74 | A | C | |
| ATOM | 1689 | CD1 | ILE | A | 827 | 54.374 | 35.272 | 15.915 | 1.00 | 38.78 | A | C | |
| ATOM | 1690 | C | ILE | A | 827 | 54.707 | 39.001 | 19.256 | 1.00 | 32.81 | A | C | |
| ATOM | 1691 | O | ILE | A | 827 | 53.750 | 39.562 | 19.787 | 1.00 | 38.91 | A | O | |
| ATOM | 1692 | N | PHE | A | 828 | 55.800 | 38.645 | 19.924 | 1.00 | 32.90 | A | N | |
| ATOM | 1693 | CA | PHE | A | 828 | 55.925 | 38.846 | 21.366 | 1.00 | 37.93 | A | C | |
| ATOM | 1694 | CB | PHE | A | 828 | 56.894 | 37.815 | 21.954 | 1.00 | 39.93 | A | C | |
| ATOM | 1695 | CG | PHE | A | 828 | 56.647 | 36.411 | 21.488 | 1.00 | 44.73 | A | C | |
| ATOM | 1696 | CD1 | PHE | A | 828 | 55.407 | 35.821 | 21.645 | 1.00 | 49.08 | A | C | |
| ATOM | 1697 | CD2 | PHE | A | 828 | 57.658 | 35.680 | 20.893 | 1.00 | 43.41 | A | C | |
| ATOM | 1698 | CE1 | PHE | A | 828 | 55.182 | 34.527 | 21.215 | 1.00 | 44.87 | A | C | |
| ATOM | 1699 | CE2 | PHE | A | 828 | 57.437 | 34.389 | 20.463 | 1.00 | 45.13 | A | C | |
| ATOM | 1700 | CZ | PHE | A | 828 | 56.199 | 33.813 | 20.625 | 1.00 | 45.05 | A | C | |
| ATOM | 1701 | C | PHE | A | 828 | 56.365 | 40.231 | 21.832 | 1.00 | 38.36 | A | C | |
| ATOM | 1702 | O | PHE | A | 828 | 56.027 | 40.642 | 22.942 | 1.00 | 38.99 | A | O | |
| ATOM | 1703 | N | ASP | A | 8.29 | 57.119 | 40.949 | 21.008 | 1.00 | 36.74 | A | N | |
| ATOM | 1704 | CA | ASP | A | 829 | 57.594 | 42.267 | 21.410 | 1.00 | 39.45 | A | C | |
| ATOM | 1705 | CB | ASP | A | 829 | 59.115 | 42.247 | 21.550 | 1.00 | 40.14 | A | C | |
| ATOM | 1706 | CG | ASP | A | 829 | 59.600 | 41.113 | 22.430 | 1.00 | 47.35 | A | C | |
| ATOM | 1707 | OD1 | ASP | A | 829 | 59.053 | 40.955 | 23.541 | 1.00 | 53.54 | A | O | |
| ATOM | 1708 | OD2 | ASP | A | 829 | 60.528 | 40.384 | 22.017 | 1.00 | 50.16 | A | O | |
| ATOM | 1709 | C | ASP | A | 829 | 57.192 | 43.399 | 20.474 | 1.00 | 42.30 | A | C | |
| ATOM | 1710 | O | ASP | A | 829 | 57.684 | 44.521 | 20.606 | 1.00 | 4220 | A | O | |
| ATOM | 1711 | N | CYS | A | 830 | 56.302 | 43.110 | 19.532 | 1.00 | 39.95 | A | N | |
| ATOM | 1712 | CA | CYS | A | 830 | 55.854 | 44.127 | 18.587 | 1.00 | 40.90 | A | C | |
| ATOM | 1713 | CB | CYS | A | 830 | 55.022 | 45.189 | 19.311 | 1.00 | 43.86 | A | C | |
| ATOM | 1714 | SG | CYS | A | 830 | 53.438 | 44.586 | 19.941 | 1.00 | 53.73 | A | S | |
| ATOM | 1715 | C | CYS | A | 830 | 57.012 | 44.803 | 17.857 | 1.00 | 39.03 | A | C | |
| ATOM | 1716 | O | CYS | A | 830 | 56.870 | 45.924 | 17.370 | 1.00 | 44.26 | A | O | |
| ATOM | 1717 | N | VAL | A | 831 | 58.150 | 44.118 | 17.780 | 1.00 | 35.38 | A | N | |
| ATOM | 1718 | CA | VAL | A | 831 | 59.331 | 44.652 | 17.103 | 1.00 | 33.09 | A | C | |
| ATOM | 1719 | CB | VAL | A | 831 | 60.628 | 44.135 | 17.753 | 1.00 | 39.10 | A | C | |
| ATOM | 1720 | CG1 | VAL | A | 831 | 61.826 | 44.512 | 16.892 | 1.00 | 38.90 | A | C | |
| ATOM | 1721 | CG2 | VAL | A | 831 | 60.778 | 44.716 | 19.158 | 1.00 | 33.47 | A | C | |
| ATOM | 1722 | C | VAL | A | 831 | 59.346 | 44.251 | 15.631 | 1.00 | 35.33 | A | C | |
| ATOM | 1723 | O | VAL | A | 831 | 59.261 | 43.070 | 15.310 | 1.00 | 37.29 | A | O | |
| ATOM | 1724 | N | TYR | A | 832 | 59.454 | 45.237 | 14.743 | 1.00 | 36.20 | A | N | |
| ATOM | 1725 | CA | TYR | A | 832 | 59.482 | 44.981 | 13.305 | 1.00 | 35.18 | A | C | |
| ATOM | 1726 | CB | TYR | A | 832 | 58.202 | 45.490 | 12.636 | 1.00 | 41.06 | A | C | |
| ATOM | 1727 | CG | TYR | A | 832 | 56.925 | 44.829 | 13.119 | 1.00 | 52.01 | A | C | |
| ATOM | 1728 | CD1 | TYR | A | 832 | 56.442 | 45.059 | 14.402 | 1.00 | 58.36 | A | C | |
| ATOM | 1729 | CE1 | TYR | A | 832 | 55.256 | 44.489 | 14.835 | 1.00 | 59.61 | A | C | |
| ATOM | 1730 | CD2 | TYR | A | 832 | 56.186 | 44.002 | 12.278 | 1.00 | 54.33 | A | C | |
| ATOM | 1731 | CE2 | TYR | A | 832 | 54.999 | 43.427 | 12.700 | 1.00 | 57.69 | A | C | |
| ATOM | 1732 | CZ | TYR | A | 832 | 54.539 | 43.676 | 13.980 | 1.00 | 64.02 | A | C | |
| ATOM | 1733 | OH | TYR | A | 832 | 53.351 | 43.125 | 14.407 | 1.00 | 69.27 | A | O | |
| ATOM | 1734 | C | TYR | A | 832 | 60.687 | 45.646 | 12.647 | 1.00 | 31.90 | A | C | |
| ATOM | 1735 | O | TYR | A | 832 | 60.797 | 46.873 | 12.610 | 1.00 | 32.89 | A | O | |
| ATOM | 1736 | N | THR | A | 833 | 61.581 | 44.825 | 12.112 | 1.00 | 27.49 | A | N | |
| ATOM | 1737 | CA | THR | A | 833 | 62.787 | 45.315 | 11.458 | 1.00 | 25.12 | A | C | |
| ATOM | 1738 | CB | THR | A | 833 | 64.014 | 45.053 | 12.320 | 1.00 | 22.61 | A | C | |
| ATOM | 1739 | CG1 | THR | A | 833 | 64.301 | 43.646 | 12.295 | 1.00 | 24.29 | A | O | |
| ATOM | 1740 | CG2 | THR | A | 833 | 63.754 | 45.482 | 13.762 | 1.00 | 23.08 | A | C | |
| ATOM | 1741 | C | THR | A | 833 | 62.991 | 44.536 | 10.170 | 1.00 | 25.15 | A | C | |
| ATOM | 1742 | O | THR | A | 833 | 62.174 | 43.683 | 9.808 | 1.00 | 22.41 | A | O | |
| ATOM | 1743 | N | VAL | A | 834 | 64.098 | 44.819 | 9.492 | 1.00 | 23.12 | A | N | |
| ATOM | 1744 | CA | VAL | A | 834 | 64.420 | 44.113 | 8.263 | 1.00 | 22.35 | A | C | |
| ATOM | 1745 | CB | VAL | A | 834 | 65.685 | 44.707 | 7.599 | 1.00 | 21.25 | A | C | |
| ATOM | 1746 | CG1 | VAL | A | 834 | 66.148 | 43.819 | 6.445 | 1.00 | 28.11 | A | C | |
| ATOM | 1747 | CG2 | VAL | A | 834 | 65.386 | 46.105 | 7.094 | 1.00 | 26.74 | A | C | |
| ATOM | 1748 | C | VAL | A | 834 | 64.650 | 42.638 | 8.603 | 1.00 | 25.22 | A | C | |
| ATOM | 1749 | O | VAL | A | 834 | 64.325 | 41.755 | 7.814 | 1.00 | 20.84 | A | O | |
| ATOM | 1750 | N | GLN | A | 835 | 65.187 | 42.365 | 9.792 | 1.00 | 22.83 | A | N | |
| ATOM | 1751 | CA | GLN | A | 835 | 65.440 | 40.985 | 10.190 | 1.00 | 23.35 | A | C | |
| ATOM | 1752 | CB | GLN | A | 835 | 66.369 | 40.935 | 11.407 | 1.00 | 26.25 | A | C | |
| ATOM | 1753 | CG | GLN | A | 835 | 67.793 | 41.419 | 11.110 | 1.00 | 31.00 | A | C | |
| ATOM | 1754 | CD | GLN | A | 835 | 68.313 | 40.927 | 9.763 | 1.00 | 42.01 | A | C | |
| ATOM | 1755 | OE1 | GLN | A | 835 | 68.009 | 41.504 | 8.715 | 1.00 | 45.65 | A | 0 | |
| ATOM | 1756 | NE2 | GLN | A | 835 | 69.086 | 39.849 | 9.784 | 1.00 | 41.70 | A | N | |
| ATOM | 1757 | C | GLN | A | 835 | 64.159 | 40.199 | 10.469 | 1.00 | 21.05 | A | C | |
| ATOM | 1758 | O | GLN | A | 835 | 64.158 | 38.958 | 10.445 | 1.00 | 22.61 | A | O | |
| ATOM | 1759 | N | SER | A | 836 | 63.079 | 40.921 | 10.751 | 1.00 | 22.49 | A | N | |
| ATOM | 1760 | CA | SER | A | 836 | 61.778 | 40.299 | 10.987 | 1.00 | 19.30 | A | C | |
| ATOM | 1761 | CB | SER | A | 836 | 60.779 | 41.326 | 11.522 | 1.00 | 17.74 | A | C | |
| ATOM | 1762 | CG | SER | A | 836 | 61.263 | 41.941 | 12.703 | 1.00 | 40.58 | A | O | |
| ATOM | 1763 | C | SER | A | 836 | 61.310 | 39.824 | 9.613 | 1.00 | 19.26 | A | C | |
| ATOM | 1764 | O | SER | A | 836 | 60.798 | 38.716 | 9.469 | 1.00 | 22.39 | A | O | |
| ATOM | 1765 | N | ASP | A | 837 | 61.476 | 40.688 | 8.612 | 1.00 | 21.60 | A | N | |
| ATOM | 1766 | CA | ASP | A | 837 | 61.086 | 40.342 | 7.2.44 | 1.00 | 20.48 | A | C | |
| ATOM | 1767 | CB | ASP | A | 837 | 61.348 | 41.499 | 6.277 | 1.00 | 24.93 | A | C | |
| ATOM | 1768 | CG | ASP | A | 837 | 60.275 | 42.585 | 6.337 | 1.00 | 24.76 | A | C | |
| ATOM | 1769 | OD1 | ASP | A | 837 | 59.162 | 42.328 | 6.843 | 1.00 | 22.05 | A | O | |
| ATOM | 1770 | OD2 | ASP | A | 837 | 60.545 | 43.703 | 5.847 | 1.00 | 19.45 | A | O | |
| ATOM | 1771 | C | ASP | A | 837 | 61.844 | 39.111 | 6.763 | 1.00 | 17.79 | A | C | |
| ATOM | 1772 | O | ASP | A | 837 | 61.300 | 38.297 | 6.009 | 1.00 | 17.43 | A | O | |
| ATOM | 1773 | N | VAL | A | 838 | 63.093 | 38.971 | 7.194 | 1.00 | 18.92 | A | N | |
| ATOM | 1774 | CA | VAL | A | 838 | 63.897 | 37.817 | 6.802 | 1.00 | 15.94 | A | C | |
| ATOM | 1775 | CB | VAL | A | 838 | 65.349 | 37.935 | 7.324 | 1.00 | 23.80 | A | C | |
| ATOM | 1776 | CG1 | VAL | A | 838 | 66.088 | 36.616 | 7.114 | 1.00 | 23.03 | A | C | |
| ATOM | 1777 | CG2 | VAL | A | 838 | 66.074 | 39.072 | 6.582 | 1.00 | 16.63 | A | C | |
| ATOM | 1778 | C | VAL | A | 838 | 63.254 | 36.523 | 7.314 | 1.00 | 16.31 | A | C | |
| ATOM | 1779 | O | VAL | A | 838 | 63.199 | 35.525 | 6.604 | 1.00 | 13.23 | A | O | |
| ATOM | 1780 | N | TRP | A | 839 | 62.774 | 36.533 | 8.554 | 1.00 | 16.33 | A | N | |
| ATOM | 1781 | CA | TRP | A | 839 | 62.102 | 35.360 | 9.094 | 1.00 | 22.16 | A | C | |
| ATOM | 1782 | CB | TRP | A | 839 | 61.590 | 35.641 | 10.512 | 1.00 | 19.02 | A | C | |
| ATOM | 1783 | CG | TRP | A | 839 | 60.768 | 34.534 | 11.110 | 1.00 | 23.68 | A | C | |
| ATOM | 1784 | CD2 | TRP | A | 839 | 61.157 | 33.668 | 12.186 | 1.00 | 23.90 | A | C | |
| ATOM | 1785 | CE2 | TRP | A | 839 | 60.081 | 32.726 | 12.425 | 1.00 | 23.27 | A | C | |
| ATOM | 1786 | CE3 | TRP | A | 839 | 62.311 | 33.551 | 12.966 | 1.00 | 20.81 | A | C | |
| ATOM | 1787 | CD1 | TRP | A | 839 | 59.502 | 34.142 | 10.750 | 1.00 | 24.49 | A | C | |
| ATOM | 1788 | NE1 | TRP | A | 839 | 59.084 | 33.100 | 11.539 | 1.00 | 19.50 | A | N | |
| ATOM | 1789 | CZ2 | TRP | A | 839 | 60.127 | 31.804 | 13.414 | 1.00 | 26.78 | A | C | |
| ATOM | 1790 | CZ3 | TRP | A | 839 | 62.356 | 32.575 | 13.944 | 1.00 | 20.57 | A | C | |
| ATOM | 1791 | CH2 | TRP | A | 839 | 61.269 | 31.713 | 14.159 | 1.00 | 22.37 | A | C | |
| ATOM | 1792 | C | TRP | A | 839 | 60.915 | 35.015 | 8.192 | 1.00 | 16.87 | A | C | |
| ATOM | 1793 | O | TRP | A | 839 | 60.767 | 33.870 | 7.754 | 1.00 | 16.83 | A | O | |
| ATOM | 1794 | N | SER | A | 840 | 60.077 | 36.016 | 7.923 | 1.00 | 13.12 | A | N | |
| ATOM | 1795 | CA | SER | A | 840 | 58.888 | 35.843 | 7.091 | 1.00 | 15.26 | A | C | |
| ATOM | 1796 | CB | SER | A | 840 | 58.155 | 37.182 | 6.913 | 1.00 | 14.60 | A | C | |
| ATOM | 1797 | CG | SER | A | 840 | 57.716 | 37.690 | 8.170 | 1.00 | 21.30 | A | O | |
| ATOM | 1798 | C | SER | A | 840 | 59.271 | 35.283 | 5.736 | 1.00 | 13.12 | A | C | |
| ATOM | 1799 | O | SER | A | 840 | 58.556 | 34.457 | 5.171 | 1.00 | 15.98 | A | O | |
| ATOM | 1800 | N | TYR | A | 841 | 60.406 | 35.733 | 5.221 | 1.00 | 15.43 | A | N | |
| ATOM | 1801 | CA | TYR | A | 841 | 60.896 | 35.247 | 3.932 | 1.00 | 13.22 | A | C | |
| ATOM | 1802 | CB | TYR | A | 841 | 62.163 | 35.995 | 3.533 | 1.00 | 14.97 | A | C | |
| ATOM | 1803 | CG | TYR | A | 841 | 62.766 | 35.479 | 2.252 | 1.00 | 14.31 | A | C | |
| ATOM | 1804 | CD1 | TYR | A | 841 | 62.286 | 35.896 | 1.015 | 1.00 | 18.92 | A | C | |
| ATOM | 1805 | CE1 | TYR | A | 841 | 62.803 | 35.380 | −0.164 | 1.00 | 21.32 | A | C | |
| ATOM | 1806 | CD2 | TYR | A | 841 | 63.784 | 34.528 | 2.274 | 1.00 | 14.13 | A | C | |
| ATOM | 1807 | CE2 | TYR | A | 841 | 64.304 | 34.002 | 1.098 | 1.00 | 19.05 | A | C | |
| ATOM | 1808 | CZ | TYR | A | 841 | 63.809 | 34.432 | −0.115 | 1.00 | 20.07 | A | C | |
| ATOM | 1809 | OH | TYR | A | 841 | 64.311 | 33.904 | −1.287 | 1.00 | 17.54 | A | O | |
| ATOM | 1810 | C | TYR | A | 841 | 61.192 | 33.741 | 4.036 | 1.00 | 13.55 | A | C | |
| ATOM | 1811 | O | TYR | A | 841 | 60.986 | 32.994 | 3.086 | 1.00 | 12.94 | A | O | |
| ATOM | 1812 | N | GLY | A | 842 | 61.687 | 33.306 | 5.191 | 1.00 | 13.24 | A | N | |
| ATOM | 1813 | CA | GLY | A | 842 | 61.953 | 31.893 | 5.403 | 1.00 | 14.97 | A | C | |
| ATOM | 1814 | C | GLY | A | 842 | 60.656 | 31.099 | 5.266 | 1.00 | 15.57 | A | C | |
| ATOM | 1815 | O | GLY | A | 842 | 60.627 | 30.025 | 4.645 | 1.00 | 15.63 | A | O | |
| ATOM | 1816 | N | ILE | A | 843 | 59.575 | 31.626 | 5.842 | 1.00 | 18.69 | A | N | |
| ATOM | 1817 | CA | ILE | A | 843 | 58.278 | 30.966 | 5.756 | 1.00 | 17.62 | A | C | |
| ATOM | 1818 | CB | ILE | A | 843 | 57.217 | 31.685 | 6.614 | 1.00 | 12.37 | A | C | |
| ATOM | 1819 | CG2 | ILE | A | 843 | 55.872 | 30.956 | 6.509 | 1.00 | 18.15 | A | C | |
| ATOM | 1820 | CG1 | ILE | A | 843 | 57.667 | 31.714 | 8.073 | 1.00 | 16.08 | A | C | |
| ATOM | 1821 | CD1 | ILE | A | 843 | 57.772 | 30.328 | 8.692 | 1.00 | 17.89 | A | C | |
| ATOM | 1822 | C | ILE | A | 843 | 57.830 | 30.954 | 4.291 | 1.00 | 16.12 | A | C | |
| ATOM | 1823 | O | ILE | A | 843 | 57.283 | 29.964 | 3.814 | 1.00 | 12.81 | A | O | |
| ATOM | 1824 | N | LEU | A | 844 | 58.072 | 32.055 | 3.586 | 1.00 | 17.71 | A | N | |
| ATOM | 1825 | CA | LEU | A | 844 | 57.706 | 32.135 | 2.172 | 1.00 | 18.20 | A | C | |
| ATOM | 1826 | CB | LEU | A | 844 | 58.045 | 33.509 | 1.605 | 1.00 | 14.27 | A | C | |
| ATOM | 1827 | CG | LEU | A | 844 | 58.152 | 33.554 | 0.077 | 1.00 | 15.77 | A | C | |
| ATOM | 1828 | CD1 | LEU | A | 844 | 56.822 | 33.146 | −0.560 | 1.00 | 20.09 | A | C | |
| ATOM | 1829 | CD2 | LEU | A | 844 | 58.547 | 34.961 | −0.347 | 1.00 | 14.60 | A | C | |
| ATOM | 1830 | C | LEU | A | 844 | 58.445 | 31.063 | 1.372 | 1.00 | 20.07 | A | C | |
| ATOM | 1831 | O | LEU | A | 844 | 57.864 | 30.437 | 0.478 | 1.00 | 20.33 | A | O | |
| ATOM | 1832 | N | LEU | A | 845 | 59.724 | 30.864 | 1.687 | 1.00 | 17.72 | A | N | |
| ATOM | 1833 | CA | LEU | A | 845 | 60.535 | 29.851 | 1.008 | 1.00 | 24.22 | A | C | |
| ATOM | 1834 | CB | LEU | A | 845 | 61.969 | 29.824 | 1.550 | 1.00 | 20.44 | A | C | |
| ATOM | 1835 | CG | LEU | A | 845 | 62.981 | 30.862 | 1.063 | 1.00 | 31.34 | A | C | |
| ATOM | 1836 | CD1 | LEU | A | 845 | 64.339 | 30.553 | 1.681 | 1.00 | 23.97 | A | C | |
| ATOM | 1837 | CD2 | LEU | A | 845 | 63.076 | 30.838 | −0.453 | 1.00 | 26.86 | A | C | |
| ATOM | 1838 | C | LEU | A | 845 | 59.900 | 28.486 | 1.222 | 1.00 | 19.15 | A | C | |
| ATOM | 1839 | O | LEU | A | 845 | 59.817 | 27.677 | 0.301 | 1.00 | 19.32 | A | O | |
| ATOM | 1840 | N | TRP | A | 846 | 59.453 | 28.230 | 2.450 | 1.00 | 19.86 | A | N | |
| ATOM | 1841 | CA | TRP | A | 846 | 58.804 | 26.963 | 2.766 | 1.00 | 19.14 | A | C | |
| ATOM | 1842 | CB | TRP | A | 846 | 58.441 | 26.908 | 4.247 | 1.00 | 18.27 | A | C | |
| ATOM | 1843 | CG | TRP | A | 846 | 57.915 | 25.577 | 4.683 | 1.00 | 25.24 | A | C | |
| ATOM | 1844 | CD2 | TRP | A | 846 | 56.539 | 25.170 | 4.728 | 1.00 | 19.58 | A | C | |
| ATOM | 1845 | CE2 | TRP | A | 846 | 56.509 | 23.853 | 5.236 | 1.00 | 22.36 | A | C | |
| ATOM | 1846 | CE3 | TRP | A | 846 | 55.331 | 25.793 | 4.394 | 1.00 | 21.99 | A | C | |
| ATOM | 1847 | CD1 | TRP | A | 846 | 58.642 | 24.524 | 5.141 | 1.00 | 16.57 | A | C | |
| ATOM | 1848 | NE1 | TRP | A | 846 | 57.804 | 23.479 | 5.477 | 1.00 | 19.47 | A | N | |
| ATOM | 1849 | CZ2 | TRP | A | 846 | 55.317 | 23.147 | 5.422 | 1.00 | 17.41 | A | C | |
| ATOM | 1850 | CZ3 | TRP | A | 846 | 54.143 | 25.089 | 4.582 | 1.00 | 22.19 | A | C | |
| ATOM | 1851 | CH2 | TRP | A | 846 | 54.150 | 23.781 | 5.092 | 1.00 | 16.03 | A | C | |
| ATOM | 1852 | C | TRP | A | 846 | 57.541 | 26.787 | 1.925 | 1.00 | 19.44 | A | C | |
| ATOM | 1853 | O | TRP | A | 846 | 57.261 | 25.689 | 1.447 | 1.00 | 23.03 | A | O | |
| ATOM | 1854 | N | GLU | A | 847 | 56.778 | 27.866 | 1.732 | 1.00 | 16.80 | A | N | |
| ATOM | 1855 | CA | GLU | A | 847 | 55.558 | 27.789 | 0.928 | 1.00 | 16.06 | A | C | |
| ATOM | 1856 | CB | GLU | A | 847 | 54.789 | 29.111 | 0.949 | 1.00 | 20.15 | A | C | |
| ATOM | 1857 | CG | GLU | A | 847 | 54.218 | 29.539 | 2.286 | 1.00 | 15.75 | A | C | |
| ATOM | 1858 | CD | GLU | A | 847 | 53.465 | 30.845 | 2.159 | 1.00 | 21.10 | A | C | |
| ATOM | 1859 | OE1 | GLU | A | 847 | 52.267 | 30.806 | 1.800 | 1.00 | 18.17 | A | O | |
| ATOM | 1860 | OE2 | GLU | A | 847 | 54.083 | 31.911 | 2.398 | 1.00 | 13.77 | A | O | |
| ATOM | 1861 | C | GLU | A | 847 | 55.920 | 27.495 | −0.524 | 1.00 | 18.01 | A | C | |
| ATOM | 1862 | O | GLU | A | 847 | 55.259 | 26.698 | −1.206 | 1.00 | 21.11 | A | O | |
| ATOM | 1863 | N | ILE | A | 848 | 56.959 | 28.167 | −1.003 | 1.00 | 16.81 | A | N | |
| ATOM | 1864 | CA | ILE | A | 848 | 57.402 | 27.984 | −2.384 | 1.00 | 17.37 | A | C | |
| ATOM | 1865 | CB | ILE | A | 848 | 58.626 | 28.869 | −2.703 | 1.00 | 22.66 | A | C | |
| ATOM | 1866 | CG2 | ILE | A | 848 | 59.292 | 28.388 | −3.987 | 1.00 | 22.09 | A | C | |
| ATOM | 1867 | CG1 | ILE | A | 848 | 58.208 | 30.339 | −2.830 | 1.00 | 21.73 | A | C | |
| ATOM | 1868 | CD1 | ILE | A | 848 | 59.361 | 31.273 | −3.168 | 1.00 | 22.82 | A | C | |
| ATOM | 1869 | C | ILE | A | 848 | 57.794 | 26.527 | −2.638 | 1.00 | 25.72 | A | C | |
| ATOM | 1870 | O | ILE | A | 848 | 57.289 | 25.885 | −3.559 | 1.00 | 19.89 | A | O | |
| ATOM | 1871 | N | PHE | A | 849 | 58.675 | 26.000 | −1.793 | 1.00 | 22.31 | A | O | |
| ATOM | 1872 | CA | PHE | A | 849 | 59.170 | 24.639 | −1.968 | 1.00 | 23.53 | A | C | |
| ATOM | 1873 | CB | PHE | A | 849 | 60.562 | 24.546 | −1.349 | 1.00 | 22.24 | A | C | |
| ATOM | 1874 | CG | PHE | A | 849 | 61.619 | 25.243 | −2.165 | 1.00 | 25.34 | A | C | |
| ATOM | 1875 | CD1 | PHE | A | 849 | 62.231 | 24.594 | −3.226 | 1.00 | 24.73 | A | C | |
| ATOM | 1876 | CD2 | PHE | A | 849 | 61.943 | 26.567 | −1.920 | 1.00 | 24.47 | A | C | |
| ATOM | 1877 | CE1 | PHE | A | 849 | 63.142 | 25.250 | −4.028 | 1.00 | 26.25 | A | C | |
| ATOM | 1878 | CE2 | PHE | A | 849 | 62.856 | 27.235 | −2.720 | 1.00 | 25.29 | A | C | |
| ATOM | 1879 | CZ | PHE | A | 849 | 63.456 | 26.577 | −3.777 | 1.00 | 30.37 | A | C | |
| ATOM | 1880 | C | PHE | A | 849 | 58.287 | 23.478 | −1.509 | 1.00 | 25.86 | A | C | |
| ATOM | 1881 | O | PHE | A | 849 | 58.706 | 22.318 | −1.561 | 1.00 | 26.78 | A | O | |
| ATOM | 1882 | N | SER | A | 850 | 57.072 | 23.793 | −1.070 | 1.00 | 24.88 | A | N | |
| ATOM | 1883 | CA | SER | A | 850 | 56.105 | 22.786 | −0.647 | 1.00 | 23.99 | A | C | |
| ATOM | 1884 | CB | SER | A | 850 | 55.638 | 23.052 | 0.788 | 1.00 | 22.00 | A | C | |
| ATOM | 1885 | CG | SER | A | 850 | 54.854 | 24.230 | 0.844 | 1.00 | 20.56 | A | O | |
| ATOM | 1886 | C | SER | A | 850 | 54.930 | 22.957 | −1.610 | 1.00 | 27.12 | A | C | |
| ATOM | 1887 | O | SER | A | 850 | 53.854 | 22.368 | −1.441 | 1.00 | 22.91 | A | O | |
| ATOM | 1888 | N | LEU | A | 851 | 55.160 | 23.777 | −2.629 | 1.00 | 20.02 | A | N | |
| ATOM | 1889 | CA | LEU | A | 851 | 54.148 | 24.089 | −3.629 | 1.00 | 22.60 | A | C | |
| ATOM | 1890 | CB | LEU | A | 851 | 53.879 | 22.878 | −4.539 | 1.00 | 25.67 | A | C | |
| ATOM | 1891 | CG | LEU | A | 851 | 55.096 | 22.384 | −5.339 | 1.00 | 23.14 | A | C | |
| ATOM | 1892 | CD1 | LEU | A | 851 | 54.645 | 21.341 | −6.348 | 1.00 | 26.96 | A | C | |
| ATOM | 1893 | CD2 | LEU | A | 851 | 55.776 | 23.549 | −6.065 | 1.00 | 28.28 | A | C | |
| ATOM | 1894 | C | LEU | A | 851 | 52.837 | 24.618 | −3.045 | 1.00 | 24.71 | A | C | |
| ATOM | 1895 | O | LEU | A | 851 | 51.753 | 24.243 | −3.478 | 1.00 | 23.23 | A | O | |
| ATOM | 1896 | N | GLY | A | 852 | 52.939 | 25.477 | −2.035 | 1.00 | 26.30 | A | N | |
| ATOM | 1897 | CA | GLY | A | 852 | 51.746 | 26.092 | −1.482 | 1.00 | 23.28 | A | C | |
| ATOM | 1898 | C | GLY | A | 852 | 50.998 | 25.533 | −0.289 | 1.00 | 20.12 | A | C | |
| ATOM | 1899 | O | GLY | A | 852 | 49.840 | 25.881 | −0.097 | 1.00 | 22.32 | A | O | |
| ATOM | 1900 | N | LEU | A | 853 | 51.610 | 24.662 | 0.502 | 1.00 | 20.10 | A | N | |
| ATOM | 1901 | CA | LEU | A | 853 | 50.914 | 24.148 | 1.675 | 1.00 | 19.94 | A | C | |
| ATOM | 1902 | CB | LEU | A | 853 | 51.711 | 23.022 | 2.336 | 1.00 | 22.26 | A | C | |
| ATOM | 1903 | CG | LEU | A | 853 | 51.908 | 21.689 | 1.613 | 1.00 | 27.30 | A | C | |
| ATOM | 1904 | CD1 | LEU | A | 853 | 52.748 | 20.769 | 2.497 | 1.00 | 24.38 | A | C | |
| ATOM | 1905 | CD2 | LEU | A | 853 | 50.557 | 21.053 | 1.315 | 1.00 | 22.60 | A | C | |
| ATOM | 1906 | C | LEU | A | 853 | 50.831 | 25.310 | 2.649 | 1.00 | 25.16 | A | C | |
| ATOM | 1907 | O | LEU | A | 853 | 51.653 | 26.225 | 2.576 | 1.00 | 19.43 | A | O | |
| ATOM | 1908 | N | ASN | A | 854 | 49.851 | 25.280 | 3.552 | 1.00 | 21.23 | A | N | |
| ATOM | 1909 | CA | ASN | A | 854 | 49.721 | 26.334 | 4.550 | 1.00 | 22.30 | A | C | |
| ATOM | 1910 | CB | ASN | A | 854 | 48.306 | 26.350 | 5.154 | 1.00 | 24.82 | A | C | |
| ATOM | 1911 | CG | ASN | A | 854 | 48.128 | 27.438 | 6.220 | 1.00 | 36.93 | A | C | |
| ATOM | 1912 | OD1 | ASN | A | 854 | 48.132 | 27.153 | 7.425 | 1.00 | 33.40 | A | O | |
| ATOM | 1913 | ND2 | ASN | A | 854 | 47.980 | 28.692 | 5.776 | 1.00 | 23.98 | A | N | |
| ATOM | 1914 | C | ASN | A | 854 | 50.762 | 26.040 | 5.627 | 1.00 | 22.19 | A | C | |
| ATOM | 1915 | 0 | ASN | A | 854 | 50.948 | 24.890 | 6.035 | 1.00 | 19.39 | A | O | |
| ATOM | 1916 | N | PRO | A | 855 | 51.478 | 27.076 | 6.084 | 1.00 | 21.60 | A | N | |
| ATOM | 1917 | CD | PRO | A | 855 | 51.417 | 28.469 | 5.608 | 1.00 | 14.78 | A | C | |
| ATOM | 1918 | CA | PRO | A | 855 | 52.506 | 26.912 | 7.114 | 1.00 | 16.21 | A | C | |
| ATOM | 1919 | CB | PRO | A | 855 | 52.968 | 28.353 | 7.362 | 1.00 | 16.48 | A | C | |
| ATOM | 1920 | CG | PRO | A | 855 | 52.765 | 29.004 | 6.015 | 1.00 | 21.98 | A | C | |
| ATOM | 1921 | C | PRO | A | 855 | 51.969 | 26.229 | 8.381 | 1.00 | 20.24 | A | C | |
| ATOM | 1922 | O | PRO | A | 855 | 50.794 | 26.401 | 8.736 | 1.00 | 16.54 | A | O | |
| ATOM | 1923 | N | TYR | A | 856 | 52.825 | 25.448 | 9.043 | 1.00 | 19.95 | A | N | |
| ATOM | 1924 | CA | TYR | A | 856 | 52.439 | 24.755 | 10.270 | 1.00 | 21.99 | A | C | |
| ATOM | 1925 | CB | TYR | A | 856 | 52.247 | 25.779 | 11.385 | 1.00 | 21.67 | A | C | |
| ATOM | 1926 | CG | TYR | A | 856 | 53.487 | 26.615 | 11.625 | 1.00 | 24.56 | A | C | |
| ATOM | 1927 | CD1 | TYR | A | 856 | 54.560 | 26.106 | 12.350 | 1.00 | 20.85 | A | C | |
| ATOM | 1928 | CE1 | TYR | A | 856 | 55.717 | 26.850 | 12.546 | 1.00 | 17.31 | A | C | |
| ATOM | 1929 | CD2 | TYR | A | 856 | 53.600 | 27.900 | 11.096 | 1.00 | 21.06 | A | C | |
| ATOM | 1930 | CE2 | TYR | A | 856 | 54.759 | 28.657 | 11.284 | 1.00 | 16.14 | A | C | |
| ATOM | 1931 | CZ | TYR | A | 856 | 55.811 | 28.119 | 12.012 | 1.00 | 20.58 | A | C | |
| ATOM | 1932 | OH | TYR | A | 856 | 56.965 | 28.834 | 12.199 | 1.00 | 25.68 | A | O | |
| ATOM | 1933 | C | TYR | A | 856 | 51.139 | 24.029 | 9.988 | 1.00 | 20.52 | A | C | |
| ATOM | 1934 | O | TYR | A | 856 | 50.136 | 24.234 | 10.666 | 1.00 | 20.51 | A | O | |
| ATOM | 1935 | N | PRO | A | 857 | 51.146 | 23.166 | 8.963 | 1.00 | 24.39 | A | N | |
| ATOM | 1936 | CD | PRO | A | 857 | 52.353 | 22.638 | 8.303 | 1.00 | 21.69 | A | C | |
| ATOM | 1937 | CA | PRO | A | 857 | 49.960 | 22.407 | 8.572 | 1.00 | 27.88 | A | C | |
| ATOM | 1938 | CB | PRO | A | 857 | 50.506 | 21.431 | 7.524 | 1.00 | 26.93 | A | C | |
| ATOM | 1939 | CG | PRO | A | 857 | 51.927 | 21.243 | 7.936 | 1.00 | 34.47 | A | C | |
| ATOM | 1940 | C | PRO | A | 857 | 49.246 | 21.722 | 9.733 | 1.00 | 27.56 | A | C | |
| ATOM | 1941 | O | PRO | A | 857 | 49.859 | 20.996 | 10.516 | 1.00 | 28.31 | A | O | |
| ATOM | 1942 | N | GLY | A | 858 | 47.947 | 21.981 | 9.846 | 1.00 | 32.13 | A | N | |
| ATOM | 1943 | CA | GLY | A | 858 | 47.158 | 21.374 | 10.906 | 1.00 | 33.96 | A | C | |
| ATOM | 1944 | C | GLY | A | 858 | 47.332 | 21.975 | 12.290 | 1.00 | 36.35 | A | C | |
| ATOM | 1945 | O | GLY | A | 858 | 46.586 | 21.630 | 13.206 | 1.00 | 36.92 | A | O | |
| ATOM | 1946 | N | ILE | A | 859 | 48.311 | 22.862 | 12.454 | 1.00 | 30.52 | A | N | |
| ATOM | 1947 | CA | ILE | A | 859 | 48.553 | 23.493 | 13.750 | 1.00 | 29.17 | A | C | |
| ATOM | 1948 | CB | ILE | A | 859 | 50.067 | 23.745 | 13.988 | 1.00 | 32.04 | A | C | |
| ATOM | 1949 | CG2 | ILE | A | 859 | 50.290 | .24.451 | 15.332 | 1.00 | 24.42 | A | C | |
| ATOM | 1950 | CG1 | ILE | A | 859 | 50.822 | 22.417 | 13.976 | 1.00 | 30.97 | A | C | |
| ATOM | 1951 | CD1 | ILE | A | 859 | 52.333 | 22.561 | 14.080 | 1.00 | 28.08 | A | C | |
| ATOM | 1952 | C | ILE | A | 859 | 47.810 | 24.822 | 13.810 | 1.00 | 31.64 | A | C | |
| ATOM | 1953 | O | ILE | A | 859 | 47.967 | 25.673 | 12.938 | 1.00 | 31.76 | A | O | |
| ATOM | 1954 | N | LEU | A | 860 | 46.987 | 24.987 | 14.838 | 1.00 | 32.83 | A | N | |
| ATOM | 1955 | CA | LEU | A | 860 | 46.214 | 26.208 | 15.007 | 1.00 | 34.46 | A | C | |
| ATOM | 1956 | CR | LEU | A | 860 | 44.906 | 25.912 | 15.746 | 1.00 | 36.28 | A | C | |
| ATOM | 1957 | CG | LEU | A | 860 | 43.882 | 24.996 | 15.078 | 1.00 | 40.12 | A | C | |
| ATOM | 1958 | CD1 | LEU | A | 860 | 42.686 | 24.820 | 16.010 | 1.00 | 45.30 | A | C | |
| ATOM | 1959 | CD2 | LEU | A | 860 | 43.444 | 25.592 | 13.750 | 1.00 | 38.38 | A | C | |
| ATOM | 1960 | C | LEU | A | 860 | 46.998 | 27.243 | 15.796 | 1.00 | 32.17 | A | C | |
| ATOM | 1961 | O | LEU | A | 860 | 47.837 | 26.900 | 16.632 | 1.00 | 30.67 | A | O | |
| ATOM | 1962 | N | VAL | A | 861 | 46.717 | 28.512 | 15.525 | 1.00 | 34.28 | A | N | |
| ATOM | 1963 | CA | VAL | A | 861 | 47.376 | 29.601 | 16.230 | 1.00 | 35.56 | A | C | |
| ATOM | 1964 | CB | VAL | A | 861 | 47.273 | 30.927 | 15.451 | 1.00 | 36.71 | A | C | |
| ATOM | 1965 | CG1 | VAL | A | 861 | 47.737 | 32.082 | 16.332 | 1.00 | 37.10 | A | C | |
| ATOM | 1966 | CG2 | VAL | A | 861 | 48.124 | 30.855 | 14.181 | 1.00 | 28.53 | A | C | |
| ATOM | 1967 | C | VAL | A | 861 | 46.699 | 29.773 | 17.587 | 1.00 | 34.67 | A | C | |
| ATOM | 1968 | O | VAL | A | 861 | 45.545 | 30.179 | 17.668 | 1.00 | 29.97 | A | O | |
| ATOM | 1969 | N | ASN | A | 862 | 47.421 | 29.428 | 18.644 | 1.00 | 38.73 | A | N | |
| ATOM | 1970 | CA | ASN | A | 862 | 46.917 | 29.552 | 20.004 | 1.00 | 40.27 | A | C | |
| ATOM | 1971 | CB | ASN | A | 862 | 45.982 | 28.386 | 20.351 | 1.00 | 36.28 | A | C | |
| ATOM | 1972 | CC | ASN | A | 862 | 46.660 | 27.030 | 20.246 | 1.00 | 42.13 | A | C | |
| ATOM | 1973 | OD1 | ASN | A | 862 | 47.805 | 26.862 | 20.659 | 1.00 | 43.57 | A | O | |
| ATOM | 1974 | ND2 | ASN | A | 862 | 45.941 | 26.048 | 19.707 | 1.00 | 46.38 | A | N | |
| ATOM | 1975 | C | ASN | A | 862 | 48.112 | 29.576 | 20.952 | 1.00 | 41.97 | A | C | |
| ATOM | 1976 | O | ASN | A | 862 | 49.248 | 29.774 | 20.517 | 1.00 | 39.05 | A | O | |
| ATOM | 1977 | N | SER | A | 863 | 47.861 | 29.368 | 22.240 | 1.00 | 43.20 | A | N | |
| ATOM | 1978 | CA | SER | A | 863 | 48.932 | 29.374 | 23.228 | 1.00 | 45.16 | A | C | |
| ATOM | 1979 | CB | SER | A | 863 | 48.369 | 29.119 | 24.632 | 1.00 | 50.70 | A | C | |
| ATOM | 1980 | CG | SER | A | 863 | 47.541 | 30.191 | 25.057 | 1.00 | 59.52 | A | O | |
| ATOM | 1981 | C | SER | A | 863 | 50.015 | 28.347 | 22.921 | 1.00 | 43.66 | A | C | |
| ATOM | 1982 | O | SER | A | 863 | 51.203 | 28.634 | 23.067 | 1.00 | 45.58 | A | O | |
| ATOM | 1983 | N | LYS | A | 864 | 49.612 | 27.151 | 22.505 | 1.00 | 37.02 | A | N | |
| ATOM | 1984 | CA | LYS | A | 864 | 50.581 | 26.107 | 22.192 | 1.00 | 39.18 | A | C | |
| ATOM | 1985 | CB | LYS | A | 864 | 49.871 | 24.821 | 21.754 | 1.00 | 44.08 | A | C | |
| ATOM | 1986 | CG | LYS | A | 864 | 49.213 | 24.029 | 22.876 | 1.00 | 52.77 | A | C | |
| ATOM | 1987 | CD | LYS | A | 864 | 48.090 | 24.810 | 23.538 | 1.00 | 61.47 | A | C | |
| ATOM | 1988 | CE | LYS | A | 864 | 47.394 | 23.970 | 24.598 | 1.00 | 61.23 | A | C | |
| ATOM | 1989 | NZ | LYS | A | 864 | 46.310 | 24.727 | 25.280 | 1.00 | 63.19 | A | N | |
| ATOM | 1990 | C | LYS | A | 864 | 51.530 | 26.563 | 21.084 | 1.00 | 39.38 | A | C | |
| ATOM | 1991 | O | LYS | A | 864 | 52.750 | 26.424 | 21.200 | 1.00 | 38.74 | A | O | |
| ATOM | 1992 | N | PHE | A | 865 | 50.959 | 27.109 | 20.014 | 1.00 | 37.13 | A | N | |
| ATOM | 1993 | CA | PHE | A | 865 | 51.738 | 27.586 | 18.876 | 1.00 | 33.62 | A | C | |
| ATOM | 1994 | CB | PHE | A | 865 | 50.827 | 28.296 | 17.880 | 1.00 | 30.62 | A | C | |
| ATOM | 1995 | CG | PHE | A | 865 | 51.551 | 28.837 | 16.679 | 1.00 | 36.53 | A | C | |
| ATOM | 1996 | CD1 | PHE | A | 865 | 52.060 | 27.983 | 15.719 | 1.00 | 35.76 | A | C | |
| ATOM | 1997 | CD2 | PHE | A | 865 | 51.729 | 30.201 | 16.516 | 1.00 | 34.37 | A | C | |
| ATOM | 1998 | CE1 | PHE | A | 865 | 52.732 | 28.483 | 14.617 | 1.00 | 39.00 | A | C | |
| ATOM | 1999 | CE2 | PHE | A | 865 | 52.399 | 30.703 | 15.418 | 1.00 | 35.76 | A | C | |
| ATOM | 2000 | CZ | PHE | A | 865 | 52.900 | 29.845 | 14.469 | 1.00 | 32.47 | A | C | |
| ATOM | 2001 | C | PHE | A | 865 | 52.840 | 28.549 | 19.304 | 1.00 | 32.63 | A | C | |
| ATOM | 2002 | O | PHE | A | 865 | 54.012 | 28.365 | 18.969 | 1.00 | 32.97 | A | O | |
| ATOM | 2003 | N | TYR | A | 866 | 52.453 | 29.587 | 20.036 | 1.00 | 32.73 | A | N | |
| ATOM | 2004 | CA | TYR | A | 866 | 53.406 | 30.582 | 20.503 | 1.00 | 33.59 | A | C | |
| ATOM | 2005 | CB | TYR | A | 866 | 52.689 | 31.614 | 21.368 | 1.00 | 34.61 | A | C | |
| ATOM | 2006 | CG | TYR | A | 866 | 51.694 | 32.430 | 20.581 | 1.00 | 30.27 | A | C | |
| ATOM | 2007 | CD1 | TYR | A | 866 | 50.368 | 32.528 | 20.979 | 1.00 | 25.95 | A | C | |
| ATOM | 2008 | CE1 | TYR | A | 866 | 49.452 | 33.251 | 20.235 | 1.00 | 33.50 | A | C | |
| ATOM | 2009 | CD2 | TYR | A | 866 | 52.078 | 33.081 | 19.421 | 1.00 | 25.86 | A | C | |
| ATOM | 2010 | CE2 | TYR | A | 866 | 51.175 | 33.806 | 18.675 | 1.00 | 28.90 | A | C | |
| ATOM | 2011 | CZ | TYR | A | 866 | 49.865 | 33.887 | 19.082 | 1.00 | 31.65 | A | C | |
| ATOM | 2012 | OH | TYR | A | 866 | 48.964 | 34.598 | 18.325 | 1.00 | 34.85 | A | O | |
| ATOM | 2013 | C | TYR | A | 866 | 54.551 | 29.936 | 21.265 | 1.00 | 33.05 | A | C | |
| ATOM | 2014 | O | TYR | A | 866 | 55.715 | 30.253 | 21.028 | 1.00 | 34.46 | A | O | |
| ATOM | 2015 | N | LYS | A | 867 | 54.221 | 29.019 | 22.168 | 1.00 | 38.38 | A | N | |
| ATOM | 2016 | CA | LYS | A | 867 | 55.238 | 28.331 | 22.946 | 1.00 | 38.06 | A | C | |
| ATOM | 2017 | CB | LYS | A | 867 | 54.590 | 27.376 | 23.949 | 1.00 | 43.78 | A | C | |
| ATOM | 2018 | CG | LYS | A | 867 | 53.756 | 28.069 | 25.015 | 1.00 | 49.88 | A | C | |
| ATOM | 2019 | CD | LYS | A | 867 | 53.301 | 27.081 | 26.080 | 1.00 | 57.21 | A | C | |
| ATOM | 2020 | CE | LYS | A | 867 | 52.597 | 27.781 | 27.235 | 1.00 | 60.27 | A | C | |
| ATOM | 2021 | NZ | LYS | A | 867 | 51.345 | 28.466 | 26.800 | 1.00 | 68.81 | A | N | |
| ATOM | 2022 | C | LYS | A | 867 | 56.163 | 27.558 | 22.019 | 1.00 | 34.75 | A | C | |
| ATOM | 2023 | O | LYS | A | 867 | 57.366 | 27.505 | 22.237 | 1.00 | 3457 | A | O | |
| ATOM | 2024 | N | LEU | A | 868 | 55.598 | 26.960 | 20.974 | 1.00 | 35.77 | A | N | |
| ATOM | 2025 | CA | LEU | A | 868 | 56.403 | 26.206 | 20.021 | 1.00 | 33.34 | A | C | |
| ATOM | 2026 | CB | LEU | A | 868 | 55.511 | 25.538 | 18.964 | 1.00 | 31.41 | A | C | |
| ATOM | 2027 | CC | LEU | A | 868 | 54.755 | 24.275 | 19.384 | 1.00 | 40.13 | A | C | |
| ATOM | 2028 | CD1 | LEU | A | 868 | 54.031 | 23.684 | 18.181 | 1.00 | 34.94 | A | C | |
| ATOM | 2029 | CD2 | LEU | A | 868 | 55.734 | 23.261 | 19.951 | 1.00 | 35.39 | A | C | |
| ATOM | 2030 | C | LEU | A | 868 | 57.432 | 27.091 | 19.325 | 1.00 | 31.07 | A | C | |
| ATOM | 2031 | O | LEU | A | 868 | 58.632 | 26.807 | 19.366 | 1.00 | 29.07 | A | O | |
| ATOM | 2032 | N | VAL | A | 869 | 56.970 | 28.160 | 18.681 | 1.00 | 31.12 | A | N | |
| ATOM | 2033 | CA | VAL | A | 869 | 57.885 | 29.053 | 17.976 | 1.00 | 35.29 | A | C | |
| ATOM | 2034 | CB | VAL | A | 869 | 57.132 | 30.180 | 17.220 | 1.00 | 33.94 | A | C | |
| ATOM | 2035 | CG1 | VAL | A | 869 | 56.199 | 29.578 | 16.193 | 1.00 | 31.92 | A | C | |
| ATOM | 2036 | CG2 | VAL | A | 869 | 56.362 | 31.043 | 18.195 | 1.00 | 41.37 | A | C | |
| ATOM | 2037 | C | VAL | A | 869 | 58.856 | 29.683 | 18.956 | 1.00 | 34.13 | A | C | |
| ATOM | 2038 | O | VAL | A | 869 | 60.028 | 29.883 | 18.642 | 1.00 | 37.82 | A | O | |
| ATOM | 2039 | N | LYS | A | 870 | 58.358 | 29.985 | 20.150 | 1.00 | 37.80 | A | N | |
| ATOM | 2040 | CA | LYS | A | 870 | 59.174 | 30.587 | 21.192 | 1.00 | 38.82 | A | C | |
| ATOM | 2041 | CB | LYS | A | 870 | 58.312 | 30.887 | 22.423 | 1.00 | 42.45 | A | C | |
| ATOM | 2042 | CG | LYS | A | 870 | 58.996 | 31.737 | 23.479 | 1.00 | 47.58 | A | C | |
| ATOM | 2043 | CD | LYS | A | 870 | 59.301 | 33.126 | 22.939 | 1.00 | 55.02 | A | C | |
| ATOM | 2044 | CB | LYS | A | 870 | 59.976 | 34.006 | 23.985 | 1.00 | 62.93 | A | C | |
| ATOM | 2045 | NZ | LYS | A | 870 | 61.304 | 33.465 | 24.401 | 1.00 | 67.77 | A | N | |
| ATOM | 2046 | C | LYS | A | 870 | 60.291 | 29.627 | 21.573 | 1.00 | 38.54 | A | C | |
| ATOM | 2047 | O | LYS | A | 870 | 61.433 | 30.042 | 21.766 | 1.00 | 39.26 | A | O | |
| ATOM | 2048 | N | ASP | A | 871 | 59.953 | 28.341 | 21.664 | 1.00 | 37.70 | A | N | |
| ATOM | 2049 | CA | ASP | A | 871 | 60.915 | 27.309 | 22.037 | 1.00 | 37.78 | A | C | |
| ATOM | 2050 | CB | ASP | A | 871 | 60.189 | 26.059 | 22.542 | 1.00 | 39.75 | A | C | |
| ATOM | 2051 | CG | ASP | A | 871 | 59.398 | 26.320 | 23.805 | 1.00 | 50.01 | A | C | |
| ATOM | 2052 | OD1 | ASP | A | 871 | 59.876 | 27.109 | 24.649 | 1.00 | 56.86 | A | O | |
| ATOM | 2053 | OD2 | ASP | A | 871 | 58.307 | 25.731 | 23.961 | 1.00 | 55.83 | A | O | |
| ATOM | 2054 | C | ASP | A | 871 | 61.887 | 26.909 | 20.931 | 1.00 | 3559 | A | C | |
| ATOM | 2055 | O | ASP | A | 871 | 62.809 | 26.127 | 21.165 | 1.00 | 33.99 | A | O | |
| ATOM | 2056 | N | GLY | A | 872 | 61.687 | 27.432 | 19.728 | 1.00 | 32.21 | A | N | |
| ATOM | 2057 | CA | GLY | A | 872 | 62.595 | 27.099 | 18.643 | 1.00 | 32.62 | A | C | |
| ATOM | 2058 | C | GLY | A | 872 | 62.099 | 26.064 | 17.649 | 1.00 | 30.80 | A | C | |
| ATOM | 2059 | O | GLY | A | 872 | 62.877 | 25.549 | 16.842 | 1.00 | 26.70 | A | O | |
| ATOM | 2060 | N | TYR | A | 873 | 60.812 | 25.746 | 17.703 | 1.00 | 27.97 | A | N | |
| ATOM | 2061 | CA | TYR | A | 873 | 60.242 | 24.775 | 16.776 | 1.00 | 26.49 | A | C | |
| ATOM | 2062 | CB | TYR | A | 873 | 58.779 | 24.512 | 17.116 | 1.00 | 26.03 | A | C | |
| ATOM | 2063 | CG | TYR | A | 873 | 58.060 | 23.675 | 16.084 | 1.00 | 27.78 | A | C | |
| ATOM | 2064 | CD1 | TYR | A | 873 | 58.084 | 22.287 | 16.146 | 1.00 | 28.59 | A | C | |
| ATOM | 2065 | CE1 | TYR | A | 873 | 57.424 | 21.519 | 15.206 | 1.00 | 31.38 | A | C | |
| ATOM | 2066 | CD2 | TYR | A | 873 | 57.355 | 24.276 | 15.042 | 1.00 | 25.17 | A | C | |
| ATOM | 2067 | CE2 | TYR | A | 873 | 56.695 | 23.514 | 14.093 | 1.00 | 26.66 | A | C | |
| ATOM | 2068 | CZ | TYR | A | 873 | 56.731 | 22.138 | 14.182 | 1.00 | 31.90 | A | C | |
| ATOM | 2069 | ON | TYR | A | 873 | 56.064 | 21.376 | 13.251 | 1.00 | 31.18 | A | O | |
| ATOM | 2070 | C | TYR | A | 873 | 60.322 | 25.321 | 15.352 | 1.00 | 25.24 | A | C | |
| ATOM | 2071 | O | TYR | A | 873 | 60.045 | 26.496 | 15.116 | 1.00 | 20.64 | A | O | |
| ATOM | 2072 | N | GLN | A | 874 | 60.696 | 24.466 | 14.408 | 1.00 | 26.48 | A | N | |
| ATOM | 2073 | CA | GLN | A | 874 | 60.794 | 24.869 | 13.012 | 1.00 | 22.55 | A | C | |
| ATOM | 2074 | CB | GLN | A | 874 | 62.263 | 24.991 | 12.598 | 1.00 | 19.94 | A | C | |
| ATOM | 2075 | CG | GLN | A | 874 | 63.035 | 26.063 | 13.37 | 1.00 | 25.03 | A | C | |
| ATOM | 2076 | CD | GLN | A | 874 | 64.453 | 26.241 | 12.854 | 1.00 | 25.93 | A | C | |
| ATOM | 2077 | OE1 | GLN | A | 874 | 65.183 | 27.147 | 13.282 | 1.00 | 26.23 | A | O | |
| ATOM | 2078 | NE2 | GLN | A | 874 | 64.852 | 25.376 | 11.931 | 1.00 | 18.93 | A | N | |
| ATOM | 2079 | C | GLN | A | 874 | 60.108 | 23.831 | 12.139 | 1.00 | 26.00 | A | C | |
| ATOM | 2080 | O | GLN | A | 874 | 60.214 | 22.632 | 12.392 | 1.00 | 23.13 | A | O | |
| ATOM | 2081 | N | MET | A | 875 | 59.393 | 24.287 | 11.120 | 1.00 | 19.52 | A | N | |
| ATOM | 2082 | CA | MET | A | 875 | 58.723 | 23.353 | 10.225 | 1.00 | 21.15 | A | C | |
| ATOM | 2083 | CB | MET | A | 875 | 58.024 | 24.093 | 9.092 | 1.00 | 19.03 | A | C | |
| ATOM | 2084 | CG | MET | A | 875 | 56.716 | 24.751 | 9.451 | 1.00 | 12.92 | A | C | |
| ATOM | 2085 | SD | MET | A | 875 | 55.991 | 25.523 | 8.009 | 1.00 | 21.85 | A | S | |
| ATOM | 2086 | CE | MET | A | 875 | 56.803 | 27.168 | 8.068 | 1.00 | 23.16 | A | C | |
| ATOM | 2087 | C | MET | A | 875 | 59.743 | 22.403 | 9.620 | 1.00 | 24.16 | A | C | |
| ATOM | 2088 | O | MET | A | 875 | 60.941 | 22.702 | 9.577 | 1.00 | 22.50 | A | O | |
| ATOM | 2089 | N | ALA | A | 876 | 59.253 | 21.267 | 9.135 | 1.00 | 23.54 | A | N | |
| ATOM | 2090 | CA | ALA | A | 876 | 60.097 | 20.262 | 8.516 | 1.00 | 22.63 | A | C | |
| ATOM | 2091 | CB | ALA | A | 876 | 59.389 | 18.910 | 8.536 | 1.00 | 20.96 | A | C | |
| ATOM | 2092 | C | ALA | A | 876 | 60.425 | 20.654 | 7.079 | 1.00 | 25.26 | A | C | |
| ATOM | 2093 | O | ALA | A | 876 | 59.705 | 21.433 | 6.455 | 1.00 | 22.17 | A | O | |
| ATOM | 2094 | N | GLN | A | 877 | 61.513 | 20.097 | 6.561 | 1.00 | 26.64 | A | N | |
| ATOM | 2095 | CA | LN | A | 877 | 61.941 | 20.363 | 5.193 | 1.00 | 24.05 | A | C | |
| ATOM | 2096 | CB | GLN | A | 877 | 63.182 | 19.528 | 4.880 | 1.00 | 25.58 | A | C | |
| ATOM | 2097 | CG | GLN | A | 877 | 63.806 | 19.795 | 3.522 | 1.00 | 25.54 | A | C | |
| ATOM | 2098 | CD | GLN | A | 877 | 64.967 | 18.862 | 3.234 | 1.00 | 36.62 | A | C | |
| ATOM | 2099 | OE1 | GLN | A | 877 | 65.651 | 18.405 | 4.149 | 1.00 | 39.06 | A | O | |
| ATOM | 2100 | NE2 | GLN | A | 877 | 65.204 | 18.587 | 1.959 | 1.00 | 35.05 | A | N | |
| ATOM | 2101 | C | GLN | A | 877 | 60.822 | 20.022 | 4.203 | 1.00 | 29.06 | A | C | |
| ATOM | 2102 | O | GLN | A | 877 | 60.254 | 18.932 | 4.251 | 1.00 | 30.62 | A | O | |
| ATOM | 2103 | N | PRO | A | 878 | 60.492 | 20.953 | 3.288 | 1.00 | 24.33 | A | N | |
| ATOM | 2104 | CD | PRO | A | 878 | 61.009 | 22.322 | 3.178 | 1.00 | 29.53 | A | C | |
| ATOM | 2105 | CA | PRO | A | 878 | 59438 | 20.725 | 2.297 | 1.00 | 25.39 | A | C | |
| ATOM | 2106 | CB | PRO | A | 878 | 59.260 | 22.100 | 1.647 | 1.00 | 30.92 | A | C | |
| ATOM | 2107 | CG | PRO | A | 878 | 59.812 | 23.044 | 2.642 | 1.00 | 22.07 | A | C | |
| ATOM | 2108 | C | PRO | A | 878 | 59.852 | 19.667 | 1.275 | 1.00 | 25.47 | A | C | |
| ATOM | 2109 | O | PRO | A | 878 | 61.037 | 19.477 | 1.006 | 1.00 | 27.51 | A | O | |
| ATOM | 2110 | N | ALA | A | 879 | 58.863 | 19.000 | 0.695 | 1.00 | 32.83 | A | N | |
| ATOM | 2111 | CA | ALA | A | 879 | 59.101 | 17.943 | −0.282 | 1.00 | 24.98 | A | C | |
| ATOM | 2112 | CB | ALA | A | 879 | 57.773 | 17.440 | −0.818 | 1.00 | 27.36 | A | C | |
| ATOM | 2113 | C | ALA | A | 879 | 60.0111 | 8.317 | −1.449 | 1.00 | 28.47 | A | C | |
| ATOM | 2114 | O | ALA | A | 879 | 60.806 | 17.495 | −1.901 | 1.00 | 24.02 | A | O | |
| ATOM | 2115 | N | PHE | A | 880 | 59.921 | 19.552 | −1.930 | 1.00 | 24.93 | A | N | |
| ATOM | 2116 | CA | PHE | A | 880 | 60.733 | 19.940 | −3.077 | 1.00 | 23.72 | A | C | |
| ATOM | 2117 | CB | PHE | A | 880 | 59.820 | 20.575 | −4.123 | 1.00 | 28.35 | A | C | |
| ATOM | 2118 | CG | PHE | A | 880 | 58.640 | 19.726 | −4.452 | 1.00 | 29.22 | A | C | |
| ATOM | 2119 | CD1 | PHE | A | 880 | 57.477 | 19.815 | −3.705 | 1.00 | 28.79 | A | C | |
| ATOM | 2120 | CD2 | PHE | A | 880 | 58.722 | 18.766 | −5.448 | 1.00 | 30.39 | A | C | |
| ATOM | 2121 | CE1 | PHE | A | 880 | 56.421 | 18.964 | −3.939 | 1.00 | 31.65 | A | C | |
| ATOM | 2122 | CE2 | PHE | A | 880 | 57.668 | 17.910 | −5.687 | 1.00 | 30.75 | A | C | |
| ATOM | 2123 | CZ | PHE | A | 880 | 56.516 | 18.007 | −4.932 | 1.00 | 33.90 | A | C | |
| ATOM | 2124 | C | PHE | A | 880 | 61.941 | 20.819 | −2.806 | 1.00 | 26.90 | A | C | |
| ATOM | 2125 | O | PHE | A | 880 | 62.569 | 21.331 | −3.734 | 1.00 | 28.62 | A | O | |
| ATOM | 2126 | N | ALA | A | 881 | 62.290 | 20.978 | −1.536 | 1.00 | 25.14 | A | N | |
| ATOM | 2127 | CA | ALA | A | 881 | 63.442 | 21.794 | −1.199 | 1.00 | 26.52 | A | C | |
| ATOM | 2128 | CB | ALA | A | 881 | 63.154 | 22.617 | 0.055 | 1.00 | 26.59 | A | C | |
| ATOM | 2129 | C | ALA | A | 881 | 64.714 | 20.974 | −0.992 | 1.00 | 29.30 | A | C | |
| ATOM | 2130 | O | ALA | A | 881 | 64.744 | 20.021 | −0.210 | 1.00 | 18.47 | A | O | |
| ATOM | 2131 | N | PRO | A | 882 | 65.776 | 21.315 | −1.729 | 1.00 | 26.57 | A | N | |
| ATOM | 2132 | CD | PRO | A | 882 | 65.770 | 22.156 | −2.939 | 1.00 | 33.69 | A | C | |
| ATOM | 2133 | CA | PRO | A | 882 | 67.048 | 20.607 | −1.588 | 1.00 | 29.31 | A | C | |
| ATOM | 2134 | CB | PRO | A | 882 | 67.908 | 21.236 | −2.678 | 1.00 | 33.45 | A | C | |
| ATOM | 2135 | CG | PRO | A | 882 | 66.910 | 21.579 | −3.730 | 1.00 | 32.98 | A | C | |
| ATOM | 2136 | C | PRO | A | 882 | 67.548 | 20.961 | −0.186 | 1.00 | 32.98 | A | C | |
| ATOM | 2137 | O | PRO | A | 882 | 67.036 | 21.902 | 0.434 | 1.00 | 27.82 | A | O | |
| ATOM | 2138 | N | LYS | A | 883 | 68.5352 | 0.232 | 0.321 | 1.00 | 32.48 | A | N | |
| ATOM | 2139 | CA | LYS | A | 883 | 69.045 | 20.523 | 1.655 | 1.00 | 34.15 | A | C | |
| ATOM | 2140 | CB | LYS | A | 883 | 70.184 | 19.567 | 2.022 | 1.00 | 41.49 | A | C | |
| ATOM | 2141 | CG | LYS | A | 883 | 70.221 | 19.210 | 3.507 | 1.00 | 44.54 | A | C | |
| ATOM | 2142 | CD | LYS | A | 883 | 70.193 | 20.457 | 4.375 | 1.00 | 54.59 | A | C | |
| ATOM | 2143 | CE | LYS | A | 883 | 69.947 | 20.127 | 5.839 | 1.00 | 60.18 | A | C | |
| ATOM | 2144 | NZ | LYS | A | 883 | 69.742 | 21.362 | 6.651 | 1.00 | 55.58 | A | N | |
| ATOM | 2145 | C | LYS | A | 883 | 69.535 | 21.968 | 1.771 | 1.00 | 32.81 | A | C | |
| ATOM | 2146 | O | LYS | A | 883 | 69.265 | 22.645 | 2.765 | 1.00 | 30.87 | A | O | |
| ATOM | 2147 | N | ASN | A | 884 | 70.252 | 22.440 | 0.756 | 1.00 | 30.41 | A | N | |
| ATOM | 2148 | CA | ASN | A | 884 | 70.776 | 23.798 | 0.770 | 1.00 | 26.43 | A | C | |
| ATOM | 2149 | CB | ASN | A | 884 | 71.555 | 24.089 | −0.513 | 1.00 | 35.44 | A | C | |
| ATOM | 2150 | CG | ASN | A | 884 | 72.857 | 23.335 | −0.582 | 1.00 | 39.22 | A | C | |
| ATOM | 2151 | OD1 | ASN | A | 884 | 73.421 | 22.956 | 0.446 | 1.00 | 45.06 | A | O | |
| ATQM | 2152 | ND2 | ASN | A | 884 | 73.359 | 23.129 | −1.794 | 1.00 | 45.29 | A | N | |
| ATOM | 2153 | C | ASN | A | 884 | 69.696 | 24.858 | 0.948 | 1.00 | 24.74 | A | C | |
| ATOM | 2154 | O | ASN | A | 884 | 69.904 | 25.850 | 1.648 | 1.00 | 24.03 | A | O | |
| ATOM | 2155 | N | ILE | A | 885 | 68.551 | 24.654 | 0.308 | 1.00 | 24.09 | A | N | |
| ATOM | 2156 | CA | ILE | A | 885 | 67.448 | 25.599 | 0.406 | 1.00 | 24.08 | A | C | |
| ATOM | 2157 | CB | .ILE | A | 885 | 66.383 | 25.318 | −0.657 | 1.00 | 23.61 | A | C | |
| ATOM | 2158 | CG2 | ILE | A | 885 | 65.220 | 26.284 | −0.509 | 1.00 | 24.48 | A | C | |
| ATOM | 2159 | CG1 | ILE | A | 885 | 67.014 | 25.448 | −2.047 | 1.00 | 20.26 | A | C | |
| ATOM | 2160 | CD1 | ILE | A | 885 | 67.693 | 26.782 | −2.275 | 1.00 | 30.31 | A | C | |
| ATOM | 2161 | C | ILE | A | 885 | 66.813 | 25.545 | 1.793 | 1.00 | 27.14 | A | C | |
| ATOM | 2162 | O | ILE | A | 885 | 66.377 | 26.568 | 2.318 | 1.00 | 21.37 | A | O | |
| ATQM | 2163 | N | TYR | A | 886 | 66.774 | 24.358 | 2.392 | 1.00 | 22.31 | A | N | |
| ATOM | 2164 | CA | TYR | A | 886 | 66.201 | 24.226 | 3.727 | 1.00 | 24.14 | A | C | |
| ATOM | 2165 | CB | TYR | A | 886 | 66.046 | 22.749 | 4.098 | 1.00 | 28.51 | A | C | |
| ATOM | 2166 | CG | TYR | A | 886 | 65.354 | 22.522 | 5.421 | 1.00 | 24.83 | A | C | |
| ATOM | 2167 | CD1 | TYR | A | 886 | 64.141 | 23.139 | 5.708 | 1.00 | 21.95 | A | C | |
| ATOM | 2168 | CE1 | TYR | A | 886 | 63.505 | 22.936 | 6.926 | 1.00 | 23.52 | A | C | |
| ATOM | 2169 | CD2 | TYR | A | 886 | 65.917 | 21.694 | 6.388 | 1.00 | 24.02 | A | C | |
| ATOM | 2170 | CE2 | TYR | A | 886 | 65.294 | 21.489 | 7.608 | 1.00 | 23.94 | A | C | |
| ATOM | 2171 | CZ | TYR | A | 886 | 64.092 | 22.109 | 7.874 | 1.00 | 26.79 | A | C | |
| ATOM | 2172 | OH | TYR | A | 886 | 63.485 | 21.913 | 9.095 | 1.00 | 23.83 | A | O | |
| ATOM | 2173 | C | TYR | A | 886 | 67.133 | 24.922 | 4.717 | 1.00 | 23.90 | A | C | |
| ATOM | 2174 | O | TYR | A | 886 | 66.700 | 25.402 | 5.764 | 1.00 | 25.15 | A | O | |
| ATOM | 2175 | N | SER | A | 887 | 68.420 | 24.972 | 4.380 | 1.00 | 18.93 | A | N | |
| ATOM | 2176 | CA | SER | A | 887 | 69.403 | 25.628 | 5.236 | 1.00 | 21.81 | A | C | |
| ATOM | 2177 | CB | SER | A | 887 | 70.824 | 25.384 | 4.713 | 1.00 | 24.01 | A | C | |
| ATOM | 2178 | CG | SER | A | 887 | 71.219 | 24.047 | 4.967 | 1.00 | 38.40 | A | O | |
| ATOM | 2179 | C | SER | A | 887 | 69.112 | 27.119 | 5.278 | 1.00 | 16.47 | A | C | |
| ATOM | 2180 | O | SER | A | 887 | 69.340 | 27.781 | 6.283 | 1.00 | 18.56 | A | O | |
| ATOM | 2181 | N | ILE | A | 888 | 68.605 | 27.651 | 4.174 | 1.00 | 15.19 | A | N | |
| ATOM | 2182 | CA | ILE | A | 888 | 68.256 | 29.061 | 4.127 | 1.00 | 18.13 | A | C | |
| ATOM | 2183 | CB | ILE | A | 888 | 67.841 | 29.484 | 2.706 | 1.00 | 21.17 | A | C | |
| ATOM | 2184 | CG2 | ILE | A | 888 | 67.434 | 30.956 | 2.698 | 1.00 | 22.33 | A | C | |
| ATOM | 2185 | CG1 | ILE | A | 888 | 69.007 | 29.250 | 1.741 | 1.00 | 15.22 | A | C | |
| ATOM | 2186 | CD1 | ILE | A | 888 | 68.685 | 29.593 | 0.295 | 1.00 | 24.73 | A | C | |
| ATOM | 2187 | C | ILE | A | 888 | 67.076 | 29.278 | 5.073 | 1.00 | 18.00 | A | C | |
| ATOM | 2188 | O | ILE | A | 888 | 67.071 | 30.207 | 5.892 | 1.00 | 19.02 | A | O | |
| ATOM | 2189 | N | MET | A | 889 | 66.079 | 28.405 | 4.961 | 1.00 | 16.44 | A | N | |
| ATOM | 2190 | CA | MET | A | 889 | 64.894 | 28.485 | 5.802 | 1.00 | 16.53 | A | C | |
| ATOM | 2191 | CB | MET | A | 889 | 63.945 | 27.305 | 5.513 | 1.00 | 21.42 | A | C | |
| ATOM | 2192 | CG | MET | A | 889 | 63.110 | 27.446 | 4.249 | 1.00 | 25.07 | A | C | |
| ATOM | 2193 | SD | MET | A | 889 | 62.123 | 25.973 | 3.860 | 1.00 | 19.76 | A | S | |
| ATOM | 2194 | CB | MET | A | 889 | 62.464 | 25.817 | 2.109 | 1.00 | 17.06 | A | C | |
| ATOM | 2195 | C | MET | A | 889 | 65.314 | 28.455 | 7.260 | 1.00 | 18.09 | A | C | |
| ATOM | 2196 | O | MET | A | 889 | 64.887 | 29.293 | 8.057 | 1.00 | 24.46 | A | O | |
| ATOM | 2197 | N | GLN | A | 890 | 66.152 | 27.484 | 7.613 | 1.00 | 20.41 | A | N | |
| ATOM | 2198 | CA | GLN | A | 890 | 66.616 | 27.352 | 8.992 | 1.00 | 21.75 | A | C | |
| ATOM | 2199 | CB | GLN | A | 890 | 67.498 | 26.110 | 9.136 | 1.00 | 20.39 | A | C | |
| ATOM | 2200 | CG | GLN | A | 890 | 66.797 | 24.793 | 8.814 | 1.00 | 21.06 | A | C | |
| ATOM | 2201 | CD | GLN | A | 890 | 67.708 | 23.596 | 9.011 | 1.00 | 22.17 | A | C | |
| ATOM | 2202 | OE1 | GLN | A | 890 | 68.802 | 23.541 | 8.455 | 1.00 | 27.55 | A | O | |
| ATOM | 2203 | NE2 | GLN | A | 890 | 67.258 | 22.630 | 9.804 | 1.00 | 25.77 | A | N | |
| ATOM | 2204 | C | GLN | A | 890 | 67.326 | 28.587 | 9.461 | 1.00 | 21.30 | A | C | |
| ATOM | 2205 | O | GLN | A | 890 | 67.284 | 28.988 | 10.624 | 1.00 | 22.93 | A | O | |
| ATOM | 2206 | N | ALA | A | 891 | 68.159 | 29.184 | 8.557 | 1.00 | 22.65 | A | N | |
| ATOM | 2207 | CA | ALA | A | 891 | 68.935 | 30.377 | 8.884 | 1.00 | 27.29 | A | C | |
| ATOM | 2208 | CB | ALA | A | 891 | 69.895 | 30.718 | 7.739 | 1.00 | 26.81 | A | C | |
| ATOM | 2209 | CA | LA | A | 891 | 67.986 | 31.539 | 9.145 | 1.00 | 20.96 | A | C | |
| ATOM | 2210 | O | ALA | A | 891 | 68.135 | 32.269 | 10.130 | 1.00 | 24.30 | A | O | |
| ATOM | 2211 | N | CYS | A | 892 | 67.009 | 31.701 | 8.257 | 1.00 | 22.95 | A | N | |
| ATOM | 2212 | CA | CYS | A | 892 | 66.008 | 32.750 | 8.387 | 1.00 | 21.58 | A | C | |
| ATOM | 2213 | CB | CYS | A | 892 | 65.021 | 32.711 | 7.220 | 1.00 | 13.34 | A | C | |
| ATOM | 2214 | SG | CYS | A | 892 | 65.691 | 33.225 | 5.626 | 1.00 | 22.09 | A | S | |
| ATOM | 2215 | C | CYS | A | 892 | 65.217 | 32.598 | 9.679 | 1.00 | 20.66 | A | C | |
| ATOM | 2216 | O | CYS | A | 892 | 64.678 | 33.579 | 10.195 | 1.00 | 24.40 | A | O | |
| ATOM | 2217 | N | TRP | A | 893 | 65.157 | 31.375 | 10.206 | 1.00 | 17.46 | A | N | |
| ATOM | 2218 | CA | TRP | A | 893 | 64.391 | 31.140 | 11.420 | 1.00 | 20.68 | A | C | |
| ATOM | 2219 | CB | TRP | A | 893 | 63.588 | 29.835 | 11.291 | 1.00 | 20.21 | A | C | |
| ATOM | 2220 | CG | TRP | A | 893 | 62.578 | 29.841 | 10.158 | 1.00 | 18.35 | A | C | |
| ATOM | 2221 | CD2 | TRP | A | 893 | 62.133 | 28.706 | 9.395 | 1.00 | 17.90 | A | C | |
| ATOM | 2222 | CE2 | TRP | A | 893 | 61.182 | 29.175 | 8.461 | 1.00 | 15.78 | A | C | |
| ATOM | 2223 | CE3 | TRP | A | 893 | 62.445 | 27.341 | 9.412 | 1.00 | 17.88 | A | C | |
| ATOM | 2224 | CD1 | TRP | A | 893 | 61.887 | 30.925 | 9.666 | 1.00 | 19.60 | A | C | |
| ATOM | 2225 | NE1 | TRP | A | 893 | 61.049 | 30.527 | 8.645 | 1.00 | 16.99 | A | N | |
| ATOM | 2226 | CZ2 | TRP | A | 893 | 60.542 | 28.324 | 7.552 | 1.00 | 21.52 | A | C | |
| ATOM | 2227 | CZ3 | TRP | A | 893 | 61.806 | 26.499 | 8.507 | 1.00 | 21.93 | A | C | |
| ATOM | 2228 | CH2 | TRP | A | 893 | 60.866 | 26.998 | 7.592 | 1.00 | 19.19 | A | C | |
| ATOM | 2229 | C | TRP | A | 893 | 65.201 | 31.148 | 12.718 | 1.00 | 20.92 | A | C | |
| ATOM | 2230 | O | TRP | A | 893 | 64.775 | 30.583 | 13.722 | 1.00 | 22.06 | A | O | |
| ATOM | 2231 | N | ALA | A | 894 | 66.367 | 31.787 | 12.702 | 1.00 | 24.33 | A | N | |
| ATOM | 223.2 | CA | ALA | A | 894 | 67.177 | 31.872 | 13.917 | 1.00 | 26.66 | A | C | |
| ATOM | 2233 | CB | ALA | A | 894 | 68.540 | 32.503 | 13.611 | 1.00 | 24.78 | A | C | |
| ATOM | 2234 | C | ALA | A | 894 | 66.388 | 32.753 | 14.886 | 1.00 | 25.66 | A | C | |
| ATOM | 2235 | O | ALA | A | 894 | 65.899 | 33.817 | 14.502 | 1.00 | 26.27 | A | O | |
| ATOM | 2236 | N | LEU | A | 895 | 66.245 | 32.307 | 16.131 | 1.00 | 23.03 | A | N | |
| ATOM | 2237 | CA | LEU | A | 895 | 65.500 | 33.070 | 17.132 | 1.00 | 28.23 | A | C | |
| ATOM | 2238 | CB | LEU | A | 895 | 65.464 | 32.307 | 18.468 | 1.00 | 25.33 | A | C | |
| ATOM | 2239 | CG | LEU | A | 895 | 64.609 | 31.032 | 18.492 | 1.00 | 30.95 | A | C | |
| ATOM | 2240 | CD1 | LEU | A | 895 | 64.633 | 30.425 | 19.888 | 1.00 | 35.97 | A | C | |
| ATOM | 2241 | CD2 | LEU | A | 895 | 63.179 | 31.360 | 18.098 | 1.00 | 31.53 | A | C | |
| ATOM | 2242 | C | LEU | A | 895 | 66.093 | 34.467 | 17.345 | 1.00 | 23.03 | A | C | |
| ATOM | 2243 | O | LEU | A | 895 | 65.367 | 35.441 | 17.542 | 1.00 | 24.63 | A | O | |
| ATOM | 2244 | N | GLU | A | 896 | 67.415 | 34.555 | 17.313 | 1.00 | 29.69 | A | N | |
| ATOM | 2245 | CA | GLU | A | 896 | 68.075 | 35.838 | 17.493 | 1.00 | 35.78 | A | C | |
| ATOM | 2246 | CB | GLU | A | 896 | 69.449 | 35.644 | 18.126 | 1.00 | 44.42 | A | C | |
| ATOM | 2247 | CG | GLU | A | 896 | 69.366 | 35.160 | 19.563 | 1.00 | 57.44 | A | C | |
| ATOM | 2248 | CD | GLU | A | 896 | 68.435 | 36.018 | 20.402 | 1.00 | 66.55 | A | C | |
| ATOM | 2249 | OE1 | GLU | A | 896 | 68.668 | 37.245 | 20.486 | 1.00 | 69.03 | A | O | |
| ATOM | 2250 | OE2 | GLU | A | 896 | 67.468 | 35.467 | 20.976 | 1.00 | 70.06 | A | O | |
| ATOM | 2251 | C | GLU | A | 896 | 68.200 | 36.510 | 16.142 | 1.00 | 29.82 | A | C | |
| ATOM | 2252 | O | GLU | A | 89.6 | 68.920 | 36.037 | 15.265 | 1.00 | 32.92 | A | O | |
| ATOM | 2253 | N | PRO | A | 897 | 67.483 | 37.626 | 15.960 | 1.00 | 28.03 | A | N | |
| ATOM | 2254 | CD | PRO | A | 897 | 66.658 | 38.2.49 | 17.007 | 1.00 | 28.24 | A | C | |
| ATOM | 2255 | CA | PRO | A | 897 | 67.453 | 38.426 | 14.734 | 1.00 | 28.53 | A | C | |
| ATOM | 2256 | CB | PRO | A | 897 | 66.733 | 39.698 | 15.173 | 1.00 | 26.92 | A | C | |
| ATOM | 2257 | CG | PRO | A | 897 | 65.786 | 39.196 | 16.203 | 1.00 | 33.03 | A | C | |
| ATOM | 2258 | C | PRO | A | 897 | 68.828 | 38.711 | 14.149 | 1.00 | 30.00 | A | C | |
| ATOM | 2259 | O | PRO | A | 897 | 69.014 | 38.656 | 12.933 | 1.00 | 28.91 | A | O | |
| ATOM | 2260 | N | THR | A | 898 | 69.791 | 39.007 | 15.019 | 1.00 | 32.64 | A | N | |
| ATOM | 2261 | CA | THR | A | 898 | 71.149 | 39.315 | 14.582 | 1.00 | 36.97 | A | C | |
| ATOM | 2262 | CB | THR | A | 898 | 72.000 | 39.861 | 15.749 | 1.00 | 36.00 | A | C | |
| ATOM | 2263 | CG1 | THR | A | 898 | 71.982 | 38.931 | 16.838 | 1.00 | 42.94 | A | C | |
| ATOM | 2264 | CG2 | THR | A | 898 | 71.444 | 41.193 | 16.225 | 1.00 | 38.53 | A | C | |
| ATOM | 2265 | C | THR | A | 898 | 71.879 | 38.135 | 13.951 | 1.00 | 30.91 | A | C | |
| ATOM | 2266 | O | THR | A | 898 | 72.833 | 38.329 | 13.199 | 1.00 | 35.82 | A | O | |
| ATOM | 2267 | N | HIS | A | 899 | 71.429 | 36.916 | 14.240 | 1.00 | 31.62 | A | N | |
| ATOM | 2268 | CA | HIS | A | 899 | 72.066 | 35.733 | 13.670 | 1.00 | 32.45 | A | C | |
| ATOM | 2269 | CB | HIS | A | 899 | 72.052 | 34.576 | 14.673 | 1.00 | 43.09 | A | C | |
| ATOM | 2270 | CG | HIS | A | 899 | 72.879 | 34.830 | 15.895 | 1.00 | 51.76 | A | C | |
| ATOM | 2271 | CD2 | HIS | A | 899 | 72.623 | 34.614 | 17.208 | 1.00 | 56.72 | A | C | |
| ATOM | 2272 | NE2 | HIS | A | 899 | 74.149 | 35.363 | 15.836 | 1.00 | 56.95 | A | N | |
| ATOM | 2273 | CE1 | HIS | A | 899 | 74.639 | 35.466 | 17.059 | 1.00 | 57.24 | A | C | |
| ATOM | 2274 | NE2 | HIS | A | 899 | 73.733 | 35.018 | 17.909 | 1.00 | 58.83 | A | N | |
| ATOM | 2275 | C | HIS | A | 899 | 71.439 | 35.284 | 12.351 | 1.00 | 33.51 | A | C | |
| ATOM | 2276 | O | HIS | A | 899 | 71.893 | 34.317 | 11.744 | 1.00 | 30.89 | A | O | |
| ATOM | 2277 | N | ARG | A | 900 | 70.393 | 35.981 | 11.910 | 1.00 | 31.73 | A | N | |
| ATOM | 2278 | CA | ARG | A | 900 | 69.750 | 35.650 | 10.637 | 1.00 | 24.76 | A | C | |
| ATOM | 2279 | CB | ARG | A | 900 | 68.322 | 36.192 | 10.594 | 1.00 | 22.49 | A | C | |
| ATOM | 2280 | CG | ARG | A | 900 | 67.396 | 35.574 | 11.608 | 1.00 | 18.10 | A | C | |
| ATOM | 2281 | CD | ARG | A | 900 | 66.068 | 36.299 | 11.652 | 1.00 | 15.39 | A | C | |
| ATOM | 2282 | NE | ARG | A | 900 | 65.331 | 35.940 | 12.862 | 1.00 | 17.02 | A | N | |
| ATOM | 2283 | CZ | ARG | A | 900 | 64.385 | 36.685 | 13.411 | 1.00 | 14.21 | A | C | |
| ATOM | 2284 | NH1 | ARG | A | 900 | 64.038 | 37.843 | 12.860 | 1.00 | 20.08 | A | N | |
| ATOM | 2285 | NH2 | ARG | A | 900 | 63.816 | 36.291 | 14.548 | 1.00 | 23.01 | A | N | |
| ATOM | 2286 | C | ARG | A | 900 | 70.560 | 36.300 | 9.522 | 1.00 | 26.28 | A | C | |
| ATOM | 2287 | O | ARG | A | 900 | 71.245 | 37.301 | 9.745 | 1.00 | 23.51 | A | O | |
| ATOM | 2288 | N | PRO | A | 901 | 70.501 | 35.737 | 8.305 | 1.00 | 19.50 | A | N | |
| ATOM | 2289 | CD | PRO | A | 901 | 69.761 | 34.530 | 7.884 | 1.00 | 23.56 | A | C | |
| ATOM | 2290 | CA | PRO | A | 901 | 71.251 | 36.313 | 7.189 | 1.00 | 22.08 | A | C | |
| ATOM | 2291 | CB | PRO | A | 901 | 71.229 | 35.198 | 6.146 | 1.00 | 17.81 | A | C | |
| ATOM | 2292 | CG | PRO | A | 901 | 69.839 | .34.609 | 6.348 | 1.00 | 18.30 | A | C | |
| ATOM | 2293 | C | PRO | A | 901 | 70.537 | 37.554 | 6.673 | 1.00 | 26.53 | A | C | |
| ATOM | 2294 | O | PRO | A | 901 | 69.357 | 37.763 | 6.952 | 1.00 | 21.69 | A | 0 | |
| ATOM | 2295 | N | THR | A | 902 | 71.251 | 38.370 | 5.911 | 1.00 | 26.52 | A | N | |
| ATOM | 2296 | CA | THR | A | 902 | 70.649 | 39.555 | 5.329 | 1.00 | 23.53 | A | C | |
| ATOM | 2297 | CB | THR | A | 902 | 71.676 | 40.674 | 5.146 | 1.00 | 22.25 | A | C | |
| ATOM | 2298 | CG1 | THR | A | 902 | 72.687 | 40.241 | 4.233 | 1.00 | 20.06 | A | O | |
| ATOM | 2299 | CG2 | THR | A | 902 | 72.314 | 41.028 | 6.476 | 1.00 | 24.06 | A | C | |
| ATOM | 2300 | C | THR | A | 902 | 70.130 | 39.122 | 3.959 | 1.00 | 28.03 | A | C | |
| ATOM | 2301 | O | THR | A | 902 | 70.529 | 38.081 | 3.438 | 1.00 | 25.18 | A | O | |
| ATOM | 2302 | N | PHE | A | 903 | 69.242 | 39.911 | 3.373 | 1.00 | 20.94 | A | N | |
| ATOM | 2303 | CA | PHE | A | 903 | 68.703 | 39.555 | 2.075 | 1.00 | 22.60 | A | C | |
| ATOM | 2304 | CB | PHE | A | 903 | 67.600 | 40.538 | 1.684 | 1.00 | 23.15 | A | C | |
| ATOM | 2305 | CG | PHE | A | 903 | 66.300 | 40.293 | 2.405 | 1.00 | 15.14 | A | C | |
| ATOM | 2306 | CD1 | PHE | A | 903 | 65.566 | 39.140 | 2.156 | 1.00 | 12.08 | A | C | |
| ATOM | 2307 | CD2 | PHE | A | 903 | 65.818 | 41.201 | 3.325 | 1.00 | 13.93 | A | C | |
| ATOM | 2308 | CE1 | PHE | A | 903 | 64.358 | 38.893 | 2.817 | 1.00 | 14.36 | A | C | |
| ATOM | 2309 | CE2 | PHE | A | 903 | 64.617 | 40.967 | 3.991 | 1.00 | 15.17 | A | C | |
| ATOM | 2310 | CZ | PHE | A | 903 | 63.883 | 39.804 | 3.734 | 1.00 | 11.14 | A | C | |
| ATOM | 2311 | C | PHE | A | 903 | 69.773 | 39.471 | 0.996 | 1.00 | 20.02 | A | C | |
| ATOM | 2312 | O | PHE | A | 903 | 69.627 | 38.713 | 0.040 | 1.00 | 20.39 | A | O | |
| ATOM | 2313 | N | GLN | A | 904 | 70.844 | 40.247 | 1.133 | 1.00 | 18.06 | A | N | |
| ATOM | 2314 | CA | GLN | A | 904 | 71.920 | 40.190 | 0.146 | 1.00 | 20.60 | A | C | |
| ATOM | 2315 | CB | GLN | A | 904 | 72.901 | 41.344 | 0.337 | 1.00 | 23.18 | A | C | |
| ATOM | 2316 | CG | GLN | A | 904 | 73.933 | 41.425 | −0.769 | 1.00 | 31.27 | A | C | |
| ATOM | 2317 | CD | GLN | A | 904 | 73.303 | 41.351 | −2.149 | 1.00 | 34.56 | A | C | |
| ATOM | 2318 | OE1 | GLN | A | 904 | 72.426 | 42.145 | −2.486 | 1.00 | 41.92 | A | O | |
| ATOM | 2319 | NE2 | GLN | A | 904 | 73.750 | 40.395 | −2.955 | 1.00 | 39.47 | A | N | |
| ATOM | 2320 | C | GLN | A | 904 | 72.650 | 38.850 | 0.292 | 1.00 | 19.48 | A | C | |
| ATOM | 2321 | O | GLN | A | 904 | 72.987 | 38.201 | −0.704 | 1.00 | 21.02 | A | O | |
| ATOM | 2322 | N | GLN | A | 905 | 72.882 | 38.435 | 1.536 | 1.00 | 20.89 | A | N | |
| ATOM | 2323 | CA | GLN | A | 905 | 73.538 | 37.157 | 1.787 | 1.00 | 20.41 | A | C | |
| ATOM | 2324 | CB | GLN | A | 905 | 73.778 | 36.967 | 3.296 | 1.00 | 22.32 | A | C | |
| ATOM | 2325 | CG | GLN | A | 905 | 75.035 | 37.712 | 3.795 | 1.00 | 20.28 | A | C | |
| ATOM | 2326 | CD | GLN | A | 905 | 75.141 | 37.818 | 5.311 | 1.00 | 2.50 | A | C | |
| ATOM | 2327 | OE1 | GLN | A | 905 | 76.192 | 38.182 | 5.846 | 1.00 | 30.15 | A | O | |
| ATOM | 2328 | NE2 | CLN | A | 905 | 74.056 | 37.521 | 6.006 | 1.00 | 17.48 | A | N | |
| ATOM | 2329 | C | GLN | A | 905 | 72.663 | 36.044 | 1.204 | 1.00 | 18.45 | A | C | |
| ATOM | 2330 | O | GLN | A | 905 | 73.173 | 35.088 | 0.632 | 1.00 | 18.96 | A | O | |
| ATOM | 2331 | N | ILE | A | 906 | 71.341 | 36.180 | 1.330 | 1.00 | 17.57 | A | N | |
| ATOM | 2332 | CA | ILE | A | 906 | 70.419 | 35.191 | 0.770 | 1.00 | 20.69 | A | C | |
| ATOM | 2333 | CB | ILE | A | 906 | 68.950 | 35.474 | 1.195 | 1.00 | 20.42 | A | C | |
| ATOM | 2334 | CG2 | ILE | A | 906 | 67.990 | 34.605 | 0.393 | 1.00 | 20.54 | A | C | |
| ATOM | 2335 | CG1 | ILE | A | 906 | 68.773 | 35.175 | 2.686 | 1.00 | 19.95 | A | C | |
| ATOM | 2336 | CD1 | ILE | A | 906 | 67.381 | 35.444 | 3.186 | 1.00 | 21.43 | A | C | |
| ATOM | 2337 | C | ILE | A | 906 | 70.490 | 35.213 | −0.761 | 1.00 | 18.82 | A | C | |
| ATOM | 2338 | O | ILE | A | 906 | 70.456 | 34.164 | −1.415 | 1.00 | 19.10 | A | O | |
| ATOM | 2339 | N | CYS | A | 907 | 70.564 | 36.415 | −1.328 | 1.00 | 24.30 | A | N | |
| ATOM | 2340 | CA | CYS | A | 907 | 70.648 | 36.584 | −2.780 | 1.00 | 25.00 | A | C | |
| ATOM | 2341 | CB | CYS | A | 907 | 70.660 | 38.072 | −3.157 | 1.00 | 23.83 | A | C | |
| ATOM | 2342 | SG | CYS | A | 907 | 69.072 | 38.922 | −3.168 | 1.00 | 24.06 | A | S | |
| ATOM | 2343 | C | CYS | A | 907 | 71.911 | 35.941 | −3.352 | 1.00 | 24.79 | A | C | |
| ATOM | 2344 | O | CYS | A | 907 | 71.875 | 35.273 | −4.390 | 1.00 | 22.55 | A | O | |
| ATOM | 2345 | N | SER | A | 908 | 73.036 | 36.169 | −2.692 | 1.00 | 23.07 | A | N | |
| ATOM | 2346 | CA | SER | A | 908 | 74.295 | 35.607 | −3.164 | 1.00 | 28.61 | A | C | |
| ATOM | 2347 | CB | SER | A | 908 | 75.476 | 36.230 | −2.411 | 1.00 | 27.95 | A | C | |
| ATOM | 2348 | CG | SER | A | 908 | 75.502 | 35.820 | −1.055 | 1.00 | 40.68 | A | O | |
| ATOM | 2349 | C | SER | A | 908 | 74.316 | 34.081 | −3.026 | 1.00 | 26.67 | A | C | |
| ATOM | 2350 | O | SER | A | 908 | 74.821 | 33.386 | −3.906 | 1.00 | 31.03 | A | O | |
| ATOM | 2351 | N | PHE | A | 909 | 73.765 | 33.550 | −1.935 | 1.00 | 29.53 | A | N | |
| ATOM | 2352 | CA | PHE | A | 909 | 73.748 | 32.097 | −1.767 | 1.00 | 27.45 | A | C | |
| ATOM | 2353 | CB | PHE | A | 909 | 73.273 | 31.695 | −0.367 | 1.00 | 26.40 | A | C | |
| ATOM | 2354 | CG | PHE | A | 909 | 73.358 | 30.212 | −0.107 | 1.00 | 27.39 | A | C | |
| ATOM | 2355 | CD1 | PHE | A | 909 | 74.572 | 29.544 | −0.207 | 1.00 | 22.67 | A | C | |
| ATOM | 2356 | CD2 | PHE | A | 909 | 72.224 | 29.484 | 0.231 | 1.00 | 27.87 | A | C | |
| ATOM | 2357 | CE1 | PHE | A | 909 | 74.657 | 28.169 | 0.027 | 1.00 | .29.86 | A | C | |
| ATOM | 2358 | CE2 | PHE | A | 909 | 72.299 | 28.115 | 0.466 | 1.00 | 30.67 | A | C | |
| ATOM | 2359 | CZ | PHE | A | 909 | 73.521 | 27.457 | 0.364 | 1.00 | 31.58 | A | C | |
| ATOM | 2360 | C | PHE | A | 909 | 72.827 | 31.477 | −2.809 | 1.00 | 30.35 | A | C | |
| ATOM | 2361 | O | PHE | A | 909 | 73.167 | 30.463 | −3.424 | 1.00 | 29.83 | A | O | |
| ATOM | 2362 | N | LEU | A | 910 | 71.660 | 32.084 | −3.013 | 1.00 | 24.69 | A | N | |
| ATOM | 2363 | CA | LEU | A | 910 | 70.719 | 31.569 | −3.998 | 1.00 | 21.53 | A | C | |
| ATOM | 2364 | CB | LEU | A | 910 | 69.422 | 32.394 | −4.013 | 1.00 | 20.17 | A | C | |
| ATOM | 2365 | CG | LEU | A | 910 | 68.373 | 32.080 | −2.933 | 1.00 | 18.50 | A | C | |
| ATOM | 2366 | CD1 | LEU | A | 910 | 67.222 | 33.113 | −3.013 | 1.00 | 15.08 | A | C | |
| ATOM | 2367 | CD2 | LEU | A | 910 | 67.836 | 30.658 | −3.119 | 1.00 | 15.80 | A | C | |
| ATOM | 2368 | C | LEU | A | 910 | 71.364 | 31.576 | −5.380 | 1.00 | 26.90 | A | C | |
| ATOM | 2369 | O | LEU | A | 910 | 71.176 | 30.641 | −6.156 | 1.OO | 26.84 | A | O | |
| ATOM | 2370 | N | GLN | A | 911 | 72.129 | 32.621 | −5.682 | 1.00 | 29.21 | A | N | |
| ATOM | 2371 | CA | GLN | A | 911 | 72.807 | 32.710 | −6.976 | 1.00 | 32.16 | A | C | |
| ATOM | 2372 | CB | GLN | A | 911 | 73.598 | 34.024 | −7.075 | 1.00 | 38.41 | A | C | |
| ATOM | 2373 | CG | GLN | A | 911 | 74.387 | 34.175 | −8.374 | 1.00 | 47.02 | A | C | |
| ATOM | 2374 | CD | GLN | A | 911 | 73.501 | 34.157 | −9.618 | 1.00 | 55.28 | A | C | |
| ATOM | 2375 | OE1 | GLN | A | 911 | 73.993 | 34.044 | −10.742 | 1.00 | 60.36 | A | O | |
| ATOM | 2376 | NE2 | GLN | A | 911 | 72.192 | 34.277 | −9.420 | 1.00 | 59.73 | A | N | |
| ATOM | 2377 | C | GLN | A | 911 | 73L747 | 31.507 | −7.157 | 1.00 | 30.03 | A | C | |
| ATOM | 2378 | O | GLN | A | 911 | 73.818 | 30.918 | −8.237 | 1.00 | 28.94 | A | O | |
| ATOM | 2379 | N | GLU | A | 912 | 74.461 | 31.149 | −6.092 | 1.00 | 26.11 | A | N | |
| ATOM | 2380 | CA | GLU | A | 912 | 75.372 | 30.007 | −6.113 | 1.00 | 28.96 | A | C | |
| ATOM | 2381 | CB | GLU | A | 912 | 76.086 | 29.866 | −4.767 | 1.00 | 28.25 | A | C | |
| ATOM | 2382 | CG | GLU | A | 912 | 77.036 | 30.994 | −4.412 | 1.00 | 31.08 | A | C | |
| ATOM | 2383 | CD | GLU | A | 912 | 77.516 | 30.885 | −2.975 | 1.00 | 43.69 | A | C | |
| ATOM | 2384 | OE1 | GLU | A | 912 | 77.869 | 29.764 | −2.554 | 1.00 | 50.89 | A | O | |
| ATOM | 2385 | OE2 | GLU | A | 912 | 77.544 | 31.915 | −2.267 | 1.00 | 51.82 | A | O | |
| ATOM | 2386 | C | GLU | A | 912 | 74.621 | 28.706 | −6.397 | 1.00 | 31.42 | A | C | |
| ATOM | 2387 | O | GLU | A | 912 | 75.142 | 27.820 | −7.071 | 1.00 | 30.82 | A | O | |
| ATOM | 2388 | N | GLN | A | 913 | 73.407 | 28.583 | −5.866 | 1.00 | 30.72 | A | N | |
| ATOM | 2389 | CA | GLN | A | 913 | 72.614 | 27.379 | −6.079 | 1.00 | 32.61 | A | C | |
| ATOM | 2390 | CB | GLN | A | 913 | 71.418 | 27.342 | −5.12 | 41.00 | 25.51 | A | C | |
| ATOM | 2391 | CG | GLN | A | 913 | 71.805 | 27.556 | −3.675 | 1.00 | 30.21 | A | C | |
| ATOM | 2392 | CD | GLN | A | 913 | 72.853 | 26.570 | −3.196 | 1.00 | 34.13 | A | C | |
| ATOM | 2393 | OE1 | GLN | A | 913 | 73.779 | 26.944 | −2.479 | 1.00 | 44.34 | A | O | |
| ATOM | 2394 | NE2 | GLN | A | 913 | 72.707 | 25.303 | −3.576 | 1.00 | 32.80 | A | N | |
| ATOM | 2395 | C | GLN | A | 913 | 72.124 | 27.322 | −7.521 | 1.00 | 33.43 | A | C | |
| ATOM | 2396 | O | GLN | A | 913 | 71.953 | 26.240 | −8.082 | 1.00 | 33.14 | A | O | |
| ATOM | 2397 | N | ALA | A | 914 | 71.899 | 28.492 | −8.111 | 1.00 | 34.61 | A | N | |
| ATOM | 2398 | CA | ALA | A | 914 | 71.433 | 28.577 | −9.492 | 1.00 | 38.93 | A | C | |
| ATOM | 2399 | CB | ALA | A | 914 | 70.892 | 29.979 | −9.782 | 1.00 | 37.54 | A | C | |
| ATOM | 2400 | C | ALA | A | 914 | 72.580 | 28.242 | −10.448 | 1.00 | 42.12 | A | C | |
| ATOM | 2401 | O | ALA | A | 914 | 72.363 | 27.660 | −11.513 | 1.00 | 41.51 | A | O | |
| ATOM | 2402 | N | GLN | A | 915 | 73.799 | 28.610 | −10.058 | 1.00 | 39.90 | A | N | |
| ATOM | 2403 | CA | GLN | A | 915 | 74.981 | 28.337 | −10.870 | 1.00 | 43.18 | A | C | |
| ATOM | 2404 | CB | GLN | A | 915 | 76.193 | 29.102 | −10.329 | 1.00 | 48.56 | A | C | |
| ATOM | 2405 | CG | GLN | A | 915 | 76.102 | 30.611 | −10.494 | 1.00 | 57.09 | A | C | |
| ATOM | 2406 | CD | GLN | A | 915 | 77.322 | 31.335 | −9.952 | 1.00 | 60.30 | A | C | |
| ATOM | 2407 | OE1 | GLN | A | 915 | 78.452 | 31.057 | −10.356 | 1.00 | 62.53 | A | O | |
| ATOM | 2408 | NE2 | GLN | A | 915 | 77.098 | 32.272 | −9.036 | 1.00 | 61.37 | A | N | |
| ATOM | 2409 | C | GLN | A | 915 | 75.282 | 26.844 | −10.874 | 1.00 | 40.29 | A | C | |
| ATOM | 2410 | O | GLN | A | 915 | 75.507 | 26.249 | −11.930 | 1.00 | 33.88 | A | O | |
| ATOM | 2411 | N | GLU | A | 916 | 75.288 | 26.244 | −9.686 | 1.00 | 40.59 | A | N | |
| ATOM | 2412 | CA | GLU | A | 916 | 75.552 | 24.816 | −9.557 | 1.00 | 44.31 | A | C | |
| ATOM | 2413 | CB | GLU | A | 916 | 75.610 | 24.421 | −8.076 | 1.00 | 43.19 | A | C | |
| ATOM | 2414 | CG | GLU | A | 916 | 75.729 | 22.920 | −7.804 | 1.00 | 49.74 | A | C | |
| ATOM | 2415 | CD | GLU | A | 916 | 76.835 | 22.240 | −8.604 | 1.00 | 51.06 | A | C | |
| ATOM | 2416 | OE1 | GLU | A | 916 | 77.945 | 22.807 | −8.710 | 1.00 | 46.82 | A | O | |
| ATOM | 2417 | OE2 | GLU | A | 916 | 76.592 | 21.125 | −9.116 | 1.00 | 41.94 | A | O | |
| ATOM | 2418 | C | GLU | A | 916 | 74.466 | 24.024 | −10.281 | 1.00 | 46.65 | A | C | |
| ATOM | 2419 | O | GLU | A | 916 | 74.757 | 23.044 | −10.969 | 1.00 | 50.03 | A | O | |
| ATOM | 2420 | N | ASP | A | 917 | 73.215 | 24.451 | −10.140 | 1.00 | 43.74 | A | N | |
| ATOM | 2421 | CA | ASP | A | 917 | 72.127 | 23.755 | −10.814 | 1.00 | 47.62 | A | C | |
| ATOM | 2422 | CB | ASP | A | 917 | 70.777 | 24.401 | −10.502 | 1.00 | 46.73 | A | C | |
| ATOM | 2423 | CG | ASP | A | 917 | 69.620 | 23.655 | −11.143 | 1.00 | 48.25 | A | C | |
| ATOM | 2424 | OD1 | ASP | A | 917 | 69.309 | 22.533 | −10.687 | 1.00 | 46.40 | A | O | |
| ATOM | 2425 | OD2 | ASP | A | 917 | 69.032 | 24.183 | −12.109 | 1.00 | 46.68 | A | O | |
| ATOM | 2426 | C | ASP | A | 917 | 72.358 | 23.792 | −12.321 | 1.00 | 48.61 | A | C | |
| ATOM | 2427 | O | ASP | A | 917 | 72.116 | 22.809 | −13.020 | 1.00 | 46.71 | A | O | |
| ATOM | 2428 | N | ARG | A | 918 | 72.826 | 24.933 | −12.817 | 1.00 | 49.75 | A | N | |
| ATOM | 2429 | CA | ARG | A | 918 | 73.085 | 25.081 | −14.241 | 1.00 | 52.53 | A | C | |
| ATOM | 2430 | CB | ARG | A | 918 | 73.283 | 26.553 | −14.603 | 1.00 | 55.45 | A | C | |
| ATOM | 2431 | CG | ARG | A | 918 | 73.549 | 26.770 | −16.080 | 1.00 | 64.49 | A | C | |
| ATOM | 2432 | CD | ARG | A | 918 | 73.685 | 28.240 | −16.424 | 1.00 | 70.23 | A | C | |
| ATOM | 2433 | NE | ARG | A | 918 | 73.998 | 28.427 | −17.838 | 1.00 | 75.64 | A | N | |
| ATOM | 2434 | CZ | ARG | A | 918 | 74.182 | 29.610 | −18.414 | 1.00 | 77.62 | A | C | |
| ATOM | 2435 | NH1 | ARG | A | 918 | 74.084 | 30.722 | −17.696 | 1.00 | 76.49 | A | N | |
| ATOM | 2436 | NH2 | ARG | A | 918 | 74.467 | 29.682 | −19.707 | 1.00 | 76.87 | A | N | |
| ATOM | 2437 | C | ARG | A | 918 | 74.319 | 24.282 | −14.650 | 1.00 | 52.64 | A | C | |
| ATOM | 2438 | 0 | ARG | A | 918 | 74.352 | 23.690 | −15.728 | 1.00 | 51.91 | A | O | |
| ATOM | 2439 | N | ARG | A | 919 | 75.333 | 24.273 | −13.790 | 1.00 | 51.34 | A | N | |
| ATOM | 2440 | CA | ARG | A | 919 | 76.558 | 23.530 | −14.068 | 1.00 | 49.04 | A | C | |
| ATOM | 2441 | CB | ARG | A | 919 | 77.519 | 23.626 | −12.886 | 1.00 | 47.65 | A | | C |
| ATOM | 2442 | CG | ARG | A | 919 | 78.862 | 22.955 | −13.121 | 1.00 | 46.84 | A | C | |
| ATOM | 2443 | CD | ARG | A | 919 | 79.589 | 22.724 | −11.806 | 1.00 | 50.42 | A | C | |
| ATOM | 2444 | NE | ARG | A | 919 | 78.954 | 21.671 | −11.016 | 1.00 | 49.33 | A | N | |
| ATOM | 2445 | CZ | ARG | A | 919 | 79.017 | 20.374 | −11.305 | 1.00 | 47.25 | A | C | |
| ATOM | 2446 | NH1 | ARG | A | 919 | 79.691 | 19.957 | −12.370 | 1.00 | 51.03 | A | N | |
| ATOM | 2447 | NH2 | ARC | A | 919 | 78.406 | 19.489 | −10.528 | 1.00 | 46.36 | A | N | |
| ATOM | 2448 | C | ARG | A | 919 | 76.180 | 22.074 | −14.286 | 1.00 | 48.85 | A | C | |
| ATOM | 2449 | O | ARG | A | 919 | 76.800 | 21.363 | −15.078 | 1.00 | 48.37 | A | O | |
| ATOM | 2450 | N | GLU | A | 920 | 75.150 | 21.644 | −13.568 | 1.00 | 48.46 | A | N | |
| ATOM | 2451 | CA | GLU | A | 920 | 74.652 | 20.280 | −13.653 | 1.00 | 54.97 | A | C | |
| ATOM | 2452 | CB | GLU | A | 920 | 73.798 | 19.970 | −12.423 | 1.00 | 52.09 | A | C | |
| ATOM | 2453 | CG | GLU | A | 920 | 74.586 | 19.894 | −11.127 | 1.00 | 50.44 | A | C | |
| ATOM | 2454 | CD | GLU | A | 920 | 73.694 | 19.801 | −9.902 | 1.00 | 53.30 | A | C | |
| ATOM | 2455 | OE1 | GLU | A | 920 | 72.618 | 19.171 | −9.992 | 1.00 | 51.23 | A | O | |
| ATOM | 2456 | OE2 | GLU | A | 920 | 74.078 | 20.343 | −8.843 | 1.00 | 51.50 | A | O | |
| ATOM | 2457 | C | GLU | A | 920 | 73.830 | 20.067 | −14.922 | 1.00 | 59.57 | A | C | |
| ATOM | 2458 | O | GLU | A | 920 | 73.985 | 19.056 | −15.606 | 1.00 | 60.63 | A | O | |
| ATOM | 2459 | N | ARG | A | 921 | 72.966 | 21.030 | −15.232 | 1.00 | 63.36 | A | N | |
| ATOM | 2460 | CA | ARG | A | 921 | 72.108 | 20.957 | −16.409 | 1.00 | 69.94 | A | C | |
| ATOM | 2461 | CB | ARG | A | 921 | 72.951 | 20.936 | −17.687 | 1.00 | 69.61 | A | C | |
| ATOM | 2462 | CD | ARG | A | 921 | 73.560 | 22.280 | −18.038 | 1.00 | 73.73 | A | C | |
| ATOM | 2463 | CD | ARG | A | 921 | 74.383 | 22.210 | −19.309 | 1.00 | 75.05 | A | C | |
| ATOM | 2464 | NE | ARG | A | 921 | 74.817 | 23.535 | −19.741 | 1.00 | 77.28 | A | N | |
| ATOM | 2465 | CZ | ARG | A | 921 | 75.552 | 23.764 | −20.824 | 1.00 | 80.16 | A | C | |
| ATOM | 2466 | NH1 | ARG | A | 921 | 75.941 | 22.751 | −21.588 | 1.00 | 80.62 | A | N | |
| ATOM | 2467 | NH2 | ARG | A | 921 | 75.895 | 25.005 | 21.147 | 1.00 | 78.40 | A | N | |
| ATOM | 2468 | C | ARG | A | 921 | 71.208 | 19.727 | −16.371 | 1.00 | 74.48 | A | C | |
| ATOM | 2469 | O | ARG | A | 921 | 69.985 | 19.904 | −16.178 | 1.00 | 76.69 | A | O | |
| ATOM | 2470 | OXT | ARG | A | 921 | 71.736 | 18.605 | 16.526 | 1.00 | 78.73 | A | O | |
| ATOM | 2471 | O | HOH | W | 1 | 53.130 | 37.517 | 6.388 | 1.00 | 16.78 | W | O | |
| ATOM | 2472 | O | HOH | W | 2 | 50.953 | 33.166 | 1.385 | 1.00 | 17.47 | W | O | |
| ATOM | 2473 | O | HOH | W | 3 | 51.687 | 43.318 | 0.792 | 1.00 | 16.68 | W | O | |
| ATOM | 2474 | O | HOH | W | 4 | 48.643 | 33.073 | 10.645 | 1.00 | 17.59 | W | O | |
| ATOM | 2475 | O | HOH | W | 5 | 54.317 | 41.896 | −1.461 | 1.00 | 13.04 | W | O | |
| ATOM | 2476 | O | HOH | W | 6 | 34.251 | 38.614 | 9.379 | 1.00 | 20.36 | W | O | |
| ATOM | 2477 | O | HOH | W | 7 | 40.992 | 38.104 | 10.054 | 1.00 | 30.20 | W | O | |
| ATOM | 2478 | O | HOH | W | 8 | 56.506 | 31.667 | 11.688 | 1.00 | 20.92 | W | O | |
| ATOM | 2479 | O | HOH | W | 9 | 48.238 | 32.734 | 2.554 | 1.00 | 20.74 | W | O | |
| ATOM | 2480 | O | HOH | W | 10 | 57.037 | 40.385 | 6.611 | 1.00 | 16.42 | W | O | |
| ATOM | 2481 | O | HOH | W | 11 | 49.789 | 22.594 | 2.865 | 1.00 | 33.34 | W | O | |
| ATOM | 2482 | 0 | HOH | W | 12 | 59.302 | 27.150 | 10.742 | 1.00 | 20.79 | W | O | |
| ATOM | 2483 | 0 | HOH | W | 13 | 52.161 | 47.340 | −16.089 | 1.00 | 34.97 | W | O | |
| ATOM | 2484 | 0 | HOH | W | 14 | 64.991 | 22.905 | 11.274 | 1.00 | 26.90 | W | O | |
| ATOM | 2485 | O | HOH | W | 15 | 50.437 | 28.565 | 1.830 | 1.00 | 16.99 | W | O | |
| ATOM | 2486 | O | HOH | W | 16 | 68.264 | 44.038 | −4.895 | 1.00 | 23.21 | W | O | |
| ATOM | 2487 | O | HOH | W | 17 | 35.950 | 39.681 | 11.160 | 1.00 | 31.94 | W | O | |
| ATOM | 2488 | O | HOH | W | 18 | 63.623 | 28.974 | 15.625 | 1.00 | 27.40 | W | O | |
| ATOM | 2489 | O | HOH | W | 19 | 54.941 | 22.104 | 11.106 | 1.00 | 25.95 | W | O | |
| ATOM | 2490 | O | HOH | W | 20 | 48.037 | 48.633 | −0.256 | 1.00 | 25.98 | W | O | |
| ATOM | 2491 | O | HOH | W | 21 | 47.589 | 22.350 | −4.545 | 1.00 | 34.50 | W | O | |
| ATOM | 2492 | O | HOH | W | 22 | 52.889 | 51.815 | 2.909 | 1.00 | 24.83 | W | O | |
| ATOM | 2493 | O | HOH | W | 23 | 50.553 | 52.673 | 4.230 | 1.00 | 29.70 | W | O | |
| ATOM | 2494 | O | HOH | W | 24 | 75.044 | 41.575 | 3.838 | 1.00 | 24.92 | W | O | |
| ATOM | 2495 | O | HOH | W | 25 | 66.112 | 46.295 | −10.986 | 1.00 | 37.12 | W | O | |
| ATOM | 2496 | O | HOH | W | 26 | 48.836 | 49.210 | −3.972 | 1.00 | 37.07 | W | O | |
| ATOM | 2497 | O | HOH | W | 27 | 61.150 | 28.769 | 16.260 | 1.00 | 33.38 | W | O | |
| ATOM | 2498 | O | HOH | W | 28 | 67.373 | 24.652 | 13.301 | 1.00 | 40.73 | W | O | |
| ATOM | 2499 | O | HOH | W | 29 | 55.269 | 32.755 | 13.628 | 1.00 | 25.27 | W | O | |
| ATOM | 2500 | O | HOH | W | 30 | 48.449 | 29.899 | 3.455 | 1.00 | 21.82 | W | O | |
| ATOM | 2501 | O | HOH | W | 31 | 58.963 | 60.604 | −2.166 | 1.00 | 42.15 | W | O | |
| ATOM | 2502 | O | HOH | W | 32 | 52.514 | 20.260 | 11.002 | 1.00 | 28.92 | W | O | |
| ATOM | 2503 | O | HOH | W | 33 | 78.761 | 30.605 | 0.100 | 1.00 | 48.74 | W | O | |
| ATOM | 2504 | O | HOH | W | 34 | 68.828 | 42.458 | 4.407 | 1.00 | 25.24 | W | O | |
| ATOM | 2505 | O | HOH | W | 35 | 65.864 | 46.954 | 10.187 | 1.00 | 31.87 | W | O | |
| ATOM | 2506 | O | HOH | W | 36 | 59.292 | 46.495 | −14.129 | 1.00 | 33.18 | W | O | |
| ATOM | 2507 | O | HOH | W | 37 | 57.824 | 39.200 | 10.857 | 1.00 | 28.93 | W | O | |
| ATOM | 2508 | O | HOH | W | 38 | 48.065 | 66.296 | −4.237 | 1.00 | 43.48 | W | O | |
| ATOM | 2509 | O | HOH | W | 39 | 63.145 | 44.635 | 4.471 | 1.00 | 22.44 | W | O | |
| ATOM | 2510 | O | HOH | W | 40 | 48.081 | 19.950 | 14.820 | 1.00 | 45.94 | W | O | |
| ATOM | 2511 | O | HOH | W | 41 | 46.013 | 36.945 | −2.606 | 1.00 | 36.92 | W | O | |
| ATOM | 2512 | O | HOH | W | 42 | 40.615 | 56.622 | −3.324 | 1.00 | 26.45 | W | O | |
| ATOM | 2513 | O | HOH | W | 43 | 70.704 | 43.006 | 2.464 | 1.00 | 24.18 | W | O | |
| ATOM | 2514 | O | HOH | W | 44 | 48.568 | 24.926 | 18.378 | 1.00 | 38.86 | W | O | |
| ATOM | 2515 | O | HOH | W | 45 | 41.885 | 39.526 | −0.221 | 1.00 | 42.04 | W | O | |
| ATOM | 2516 | O | HOH | W | 47 | 53.710 | 63.428 | −13.174 | 1.00 | 35.51 | W | O | |
| ATOM | 2517 | O | HOH | W | 48 | 68.350 | 28.106 | 13.242 | 1.00 | 40.78 | W | O | |
| ATOM | 2518 | O | HOH | W | 49 | 53.661 | 24.198 | 22.697 | 1.00 | 48.28 | W | O | |
| ATOM | 2519 | O | HOH | W | 50 | 56.511 | 63.081 | 1.177 | 1.00 | 38.76 | W | O | |
| ATOM | 2520 | O | HOH | W | 51 | 64.196 | 42.225 | 14.363 | 1.00 | 31.79 | W | O | |
| ATOM | 2521 | O | HOH | W | 52 | 52.009 | 42.073 | −16.697 | 1.00 | 35.92 | W | O | |
| ATOM | 2522 | O | HOH | W | 53 | 73.866 | 38.527 | 8.774 | 1.00 | 36.41 | W | O | |
| ATOM | 2523 | O | HOH | W | 54 | 41.590 | 64.060 | −16.394 | 1.00 | 45.45 | W | O | |
| ATOM | 2524 | O | HOH | W | 55 | 61.088 | 36.665 | 20.070 | 1.00 | 46.38 | W | O | |
| ATOM | 2525 | O | HOH | W | 56 | 86.567 | 24.345 | −7.738 | 1.00 | 33.70 | W | O | |
| ATOM | 2526 | O | HOH | W | 57 | 38.680 | 36.279 | 9.456 | 1.00 | 32.71 | W | O | |
| ATOM | 2527 | O | HOH | W | 58 | 60.903 | 33.476 | −13.064 | 1.00 | 36.11 | W | O | |
| ATOM | 2528 | O | HOH | W | 59 | 64.147 | 39.403 | −11.899 | 1.00 | 33.79 | W | O | |
| ATOM | 2529 | O | HOH | W | 60 | 67.949 | 32.224 | 21.171 | 1.00 | 48.72 | W | O | |
| ATOM | 2530 | O | HOH | W | 61 | 42.520 | 32.758 | 3.413 | 1.00 | 32.41 | W | O | |
| ATOM | 2531 | O | HOH | W | 62 | 52.963 | 44.524 | 3.043 | 1.00 | 17.47 | W | O | |
| ATOM | 2532 | O | HOH | W | 63 | 61.766 | 21.908 | 15.088 | 1.00 | 36.99 | W | O | |
| ATOM | 2533 | O | HOH | W | 64 | 55.717 | 18.349 | 14.664 | 1.00 | 55.46 | W | O | |
| ATOM | 2534 | O | HOH | W | 65 | 67.426 | 30.120 | 17.075 | 1.00 | 37.50 | W | O | |
| ATOM | 2535 | O | HOH | W | 67 | 69.917 | 35.615 | −5.993 | 1.00 | 36.41 | W | O | |
| ATOM | 2536 | O | HOH | W | 68 | 54.496 | 38.583 | 8.180 | 1.00 | 21.17 | W | O | |
| ATOM | 2537 | O | HOH | W | 69 | 71.247 | 21.615 | −3.361 | 1.00 | 47.18 | W | O | |
| ATOM | 2538 | O | HOH | W | 70 | 54.227 | 59.657 | −15.054 | 1.00 | 48.41 | W | O | |
| ATOM | 2539 | O | HOH | W | 72 | 40.518 | 35.620 | 11.660 | 1.00 | 38.50 | W | O | |
| ATOM | 2540 | O | HOH | W | 73 | 50.224 | 20.085 | −1.802 | 1.00 | 35.93 | W | O | |
| ATOM | 2541 | O | HOH | W | 74 | 35.301 | 38.742 | 1.293 | 1.00 | 27.86 | W | O | |
| ATOM | 2542 | O | HOH | W | 75 | 69.301 | 45.907 | −7.120 | 1.00 | 45.66 | W | O | |
| ATOM | 2543 | O | HOH | W | 76 | 63.459 | 41.059 | −14.066 | 1.00 | 44.45 | W | O | |
| ATOM | 2544 | O | HOH | W | 77 | 58.399 | 42.178 | 9.822 | 1.00 | 41.04 | W | O | |
| ATOM | 2545 | O | HOH | W | 78 | 62.934 | 32.029 | 25.719 | 1.00 | 57.82 | W | O | |
| ATOM | 2546 | O | HOH | W | 79 | 49.343 | 18.458 | −3.666 | 1.00 | 39.29 | W | O | |
| ATOM | 2547 | O | HOH | W | 80 | 52.614 | 18.818 | 13.004 | 1.00 | 30.64 | W | O | |
| ATOM | 2548 | O | HOH | W | 81 | 53.030 | 18.358 | −3.594 | 1.00 | 55.10 | W | O | |
| ATOM | 2549 | O | HOH | W | 82 | 65.512 | 43.135 | 16.549 | 1.00 | 31.93 | W | O | |
| ATOM | 2550 | O | HOH | W | 84 | 37.925 | 61.497 | −16.034 | 1.00 | 43.12 | W | O | |
| ATOM | 2551 | O | HOH | W | 85 | 59.955 | 48.188 | 16.163 | 1.00 | 55.43 | W | O | |
| ATOM | 2552 | O | HOH | W | 86 | 34.410 | 45.858 | −4.473 | 1.00 | 33.23 | W | O | |
| ATOM | 2553 | O | HOH | W | 87 | 59.904 | 70.813 | −7.124 | 1.00 | 41.09 | W | O | |
| ATOM | 2554 | O | HOH | W | 88 | 50.889 | 60.579 | 4.573 | 1.00 | 45.40 | W | O | |
| ATOM | 2555 | O | HOH | W | 89 | 64.444 | 21.664 | 13.862 | 1.00 | 54.06 | W | O | |
| ATOM | 2556 | O | HOH | W | 90 | 46.251 | 33.776 | −2.174 | 1.00 | 33.07 | W | O | |
| ATOM | 2557 | O | HOH | W | 91 | 46.129 | 44.410 | 10.199 | 1.00 | 43.98 | W | O | |
| ATOM | 2558 | O | HOH | W | 92 | 39.709 | 44.441 | −17.565 | 1.00 | 42.46 | W | O | |
| ATOM | 2559 | O | HOH | W | 93 | 45.261 | 29.526 | 12.722 | 1.00 | 32.17 | W | O | |
| ATOM | 2560 | O | HOH | W | 94 | 42.520 | 35.689 | 18.354 | 1.00 | 46.76 | W | O | |
| ATOM | 2561 | O | HOH | W | 97 | 54.260 | 70.596 | 0.356 | 1.00 | 46.35 | W | O | |
| ATOM | 2562 | O | HOH | W | 98 | 49.388 | 43.122 | 3.337 | 1.00 | 36.21 | W | O | |
| ATOM | 2563 | O | HOH | W | 99 | 67.754 | 32.093 | −9.558 | 1.00 | 42.69 | W | O | |
| ATOM | 2564 | O | HOH | W | 100 | 66.909 | 43.021 | 13.592 | 1.00 | 54.19 | W | O | |
| ATOM | 2565 | O | HOH | W | 101 | 46.086 | 33.428 | 0.323 | 1.00 | 32.34 | W | O | |
| ATOM | 2566 | O | HOH | W | 102 | 55.031 | 18.523 | 18.001 | 1.00 | 54.57 | W | O | |
| ATOM | 2567 | O | HOH | W | 103 | 52.486 | 42.478 | 17.047 | 1.00 | 43.11 | W | O | |
| ATOM | 2568 | O | HOH | W | 104 | 41.204 | 63.742 | −3.408 | 1.00 | 40.39 | W | O | |
| ATOM | 2569 | O | HOH | W | 105 | 48.004 | 61.755 | 4.399 | 1.00 | 43.45 | W | O | |
| ATOM | 2576 | O | HOH | W | 106 | 57.334 | 23.320 | 22.386 | 1.00 | 53.88 | W | O | |
| ATOM | 2571 | O | HOH | W | 107 | 46.410 | 22.733 | 16.519 | 1.00 | 42.34 | W | O | |
| ATOM | 2572 | O | HOH | W | 108 | 45.448 | 25.098 | 3.981 | 1.00 | 53.09 | W | O | |
| ATOM | 2573 | O | HOH | W | 109 | 48.602 | 32.712 | −10.241 | 1.00 | 37.44 | W | O | |
| ATOM | 2574 | O | HOH | W | 110 | 70.149 | 20.304 | −5.269 | 1.00 | 52.07 | W | O | |
| ATOM | 2575 | O | HOH | W | 111 | 69.497 | 46.132 | −4.008 | 1.00 | 46.09 | W | O | |
| ATOM | 2576 | O | HOH | W | 112 | 60.089 | 24.441 | −16.513 | 1.00 | 46.40 | W | O | |
| ATOM | 2577 | O | HOH | W | 114 | 55.073 | 36.156 | −21.044 | 1.00 | 37.67 | W | O | |
| ATOM | 2578 | O | HOH | W | 115 | 68.264 | 63.998 | 8.098 | 1.00 | 31.90 | W | O | |
| ATOM | 2579 | O | HOH | W | 116 | 61.712 | 62.385 | 8.248 | 1.00 | 33.65 | W | O | |
| ATOM | 2580 | O | HOH | W | 117 | 51.057 | 67.571 | −1.805 | 1.00 | 35.85 | W | O | |
| ATOM | 2581 | O | HOH | W | 118 | 34.380 | 56.720 | 7.004 | 1.00 | 44.22 | W | O | |
| ATOM | 2582 | O | HOH | W | 119 | 47.634 | 51.389 | 1.983 | 1.00 | 38.05 | W | O | |
| ATOM | 2583 | O | HOH | W | 120 | 60.173 | 56.055 | −6.963 | 1.00 | 39.76 | W | O | |
| ATOM | 2584 | O | HOH | W | 121 | 60.586 | 42.693 | −14.171 | 1.00 | 42.46 | W | O | |
| ATOM | 2585 | O | HOH | W | 122 | 53.065 | 48.717 | −5.634 | 1.00 | 31.80 | W | O | |
| ATOM | 2586 | O | HOH | W | 123 | 53.276 | 49.304 | −2.592 | 1.00 | 37.09 | W | O | |
| ATOM | 2587 | O | HOH | W | 124 | 48.023 | 23.550 | −0.097 | 1.00 | 41.97 | W | O | |
| ATOM | 2588 | O | HOH | W | 125 | 58.569 | 20.629 | −8.697 | 1.00 | 46.50 | W | O | |
| ATOM | 2589 | O | HOH | W | 126 | 62.960 | 21.885 | −15.994 | 1.00 | 39.18 | W | O | |
| ATOM | 2590 | O | HOH | W | 127 | 61.522 | 26.664 | −16.740 | 1.00 | 54.79 | W | O | |
| ATOM | 2591 | O | HOH | W | 128 | 62.573 | 29.843 | −14.065 | 1.00 | 51.60 | W | O | |
| ATOM | 2592 | O | HOH | W | 129 | 68.436 | 42.941 | −8.574 | 1.00 | 38.26 | W | O | |
| ATOM | 2593 | O | HOH | W | 130 | 70.619 | 38.577 | −10.230 | 1.00 | 43.17 | W | O | |
| ATOM | 2594 | O | HOH | W | 131 | 68.700 | 39.175 | −13.625 | 1.00 | 49.45 | W | O | |
| ATOM | 2595 | O | NOH | W | 132 | 66.629 | 35.031 | −10.564 | 1.00 | 57.28 | W | O | |
| ATOM | 2596 | O | NOH | W | 133 | 70.343 | 44.945 | 0.851 | 1.00 | 27.47 | W | O | |
| ATOM | 2597 | O | HOH | W | 134 | 71.454 | 47.029 | 2.043 | 1.00 | 32.04 | W | O | |
| ATOM | 2598 | O | HOH | W | 135 | 50.581 | 45.877 | 3.288 | 1.00 | 35.96 | W | O | |
| ATOM | 2599 | O | HOH | W | 136 | 63.043 | 18.511 | 8.678 | 1.00 | 25.84 | W | O | |
| ATOM | 2600 | O | HOH | W | 137 | 60.187 | 16.741 | 5.480 | 1.00 | 27.83 | W | O | |
| ATOM | 2601 | O | HOH | W | 138 | 71.493 | 20.508 | −1.148 | 1.00 | 37.36 | W | O | |
| ATOM | 2602 | O | HOH | W | 140 | 69.236 | 19.813 | 9.718 | 1.00 | 51.17 | W | O | |
| ATOM | 2603 | O | HOH | W | 141 | 48.893 | 48.830 | 3.386 | 1.00 | 31.72 | W | O | |
| ATOM | 2604 | O | HOH | W | 142 | 53.757 | 53.057 | 7.181 | 1.00 | 38.75 | W | O | |
| ATOM | 2605 | O | HOH | W | 143 | 52.957 | 52.603 | 10.710 | 1.00 | 45.21 | W | O | |
| ATOM | 2606 | O | HOH | W | 144 | 57.295 | 55.219 | 6.489 | 1.00 | 28.18 | W | O | |
| ATOM | 2607 | O | HOH | W | 145 | 47.242 | 19.725 | −4.341 | 1.00 | 39.04 | W | O | |
| ATOM | 2608 | O | HOH | W | 146 | 51.096 | 17.612 | −5.348 | 1.00 | 33.80 | W | O | |
| ATOM | 2609 | O | HOH | W | 147 | 45.841 | 22.498 | −11.215 | 1.00 | 41.64 | W | O | |
| ATOM | 2610 | O | HOH | W | 148 | 52.557 | 20.540 | −12.172 | 1.00 | 35.37 | W | O | |
| ATOM | 2611 | O | HOH | W | 149 | 50.830 | 28.325 | −16.387 | 1.00 | 48.81 | W | O | |
| ATOM | 2612 | O | HOH | W | 150 | 50.973 | 47.523 | −5.037 | 1.00 | 33.85 | W | O | |
| ATOM | 2613 | O | HOH | W | 151 | 79.855 | 24.442 | −8.033 | 1.00 | 34.88 | W | O | |
| ATOM | 2614 | O | HOH | W | 152 | 74.432 | 23.710 | −5.162 | 1.00 | 40.89 | W | O | |
| ATOM | 2615 | O | HOH | W | 153 | 50.051 | 65.010 | 0.979 | 1.00 | 33.32 | W | O | |
| ATOM | 2616 | O | HOH | W | 154 | 38.623 | 33.179 | 4.658 | 1.00 | 32.85 | W | O | |
| ATOM | 2617 | O | HOH | W | 155 | 69.899 | 38.414 | 18.457 | 1.00 | 42.55 | W | O | |
| ATOM | 2618 | O | HOH | W | 156 | 65.325 | 45.942 | 17.414 | 1.00 | 40.36 | W | O | |
| ATOM | 2619 | O | HOH | W | 157 | 69.467 | 32.282 | 17.501 | 1.00 | 37.72 | W | O | |
| ATOM | 2620 | O | HOH | W | 158 | 58.000 | 50.791 | −14.533 | 1.00 | 45.67 | W | O | |
| ATOM | 2621 | O | HOH | W | 159 | 69.300 | 53.882 | 1.823 | 1.00 | 39.21 | W | O | |
| ATOM | 2622 | O | HOH | W | 160 | 46.458 | 34.361 | 19.260 | 1.00 | 39.17 | W | O | |
| ATOM | 2623 | O | HOH | W | 161 | 50.935 | 53.555 | 6.466 | 1.00 | 40.79 | W | O | |
| ATOM | 2624 | O | HOH | W | 162 | 59.656 | 19.798 | 12.641 | 1.00 | 41.51 | W | O | |
| ATOM | 2625 | O | HOH | W | 163 | 34.964 | 45.762 | 2.282 | 1.00 | 37.11 | W | O | |
| ATOM | 2626 | O | HOH | W | 164 | 55.614 | 21.617 | −11.244 | 1.00 | 44.43 | W | O | |
| ATOM | 2627 | O | HOH | W | 165 | 40.312 | 33.555 | 2.156 | 1.00 | 40.12 | W | O | |
| ATOM | 2628 | O | HOH | W | 166 | 73.397 | 43.749 | 3.369 | 1.00 | 29.96 | W | O | |
| ATOM | 2629 | O | HOH | W | 167 | 71.531 | 44.662 | −1.725 | 1.00 | 35.10 | W | O | |
| ATOM | 2630 | O | NOH | W | 168 | 49.054 | 18.937 | 11.992 | 1.00 | 35.78 | W | O | |
| ATOM | 2631 | O | NOH | W | 169 | 66.761 | 18.426 | 6.769 | 1.00 | 32.23 | W | O | |
| ATOM | 2632 | O | HOH | W | 170 | 43.495 | 50.999 | 7.967 | 1.00 | 51.70 | W | O | |
| ATOM | 2633 | O | HOH | W | 171 | 87.329 | 26.319 | −6.519 | 1.00 | 35.59 | W | O | |
| ATOM | 2634 | O | NOH | W | 172 | 76.577 | 26.317 | −2.779 | 1.00 | 41.90 | W | O | |
| ATOM | 2635 | O | HOH | W | 173 | 37.878 | 56.033 | −4.242 | 1.00 | 33.32 | W | O | |
| ATOM | 2636 | O | HOH | W | 174 | 52.737 | 50.685 | 12.709 | 1.00 | 46.74 | W | O | |
| ATOM | 2637 | O | HOH | W | 175 | 53.275 | 41.894 | 21.149 | 1.00 | 44.72 | W | O | |
| ATOM | 2638 | O | HOH | W | 176 | 62.060 | 15.782 | 6.890 | 1.00 | 38.60 | W | O | |
| ATOM | 2639 | O | HOH | W | 177 | 45.643 | 66.526 | −12.547 | 1.00 | 47.05 | W | O | |
| ATOM | 2640 | O | HOH | W | 178 | 49.253 | 64.019 | 3.022 | 1.00 | 40.35 | W | O | |
| ATOM | 2641 | O | HOH | W | 179 | 50.188 | 67.986 | −4.184 | 1.00 | 35.76 | W | O | |
| ATOM | 2642 | O | HOH | W | 180 | 76.840 | 34.206 | −5.474 | 1.00 | 44.64 | W | O | |
| ATOM | 2643 | O | HOH | W | 181 | 76.534 | 38.632 | −0.025 | 1.00 | 33.48 | W | O | |
| ATOM | 2644 | O | HOH | W | 182 | 67.088 | 22.108 | 13.527 | 1.00 | 42.45 | W | O | |
| ATOM | 2645 | O | HOH | W | 183 | 42.575 | 45.889 | 4.026 | 1.00 | 32.36 | W | O | |
| ATOM | 2646 | O | HOH | W | 184 | 38.466 | 42.501 | 4.663 | 1.00 | 33.28 | W | O | |

| | | | | | | | | | | | |
|---|---|---|---|---|---|---|---|---|---|---|---|
| ATOM | 2647 | C1 | INH | I1000 | 45.826 | 40.299 | −8.534 | 1.00 | 31.13 | I | C |
| ATOM | 2648 | C2 | INH | I1000 | 45.170 | 39.086 | −8.952 | 1.00 | 29.33 | I | C |
| ATOM | 2649 | C3 | INH | I1000 | 45.271 | 38.614 | −10.286 | 1.00 | 30.96 | I | C |
| ATOM | 2650 | C4 | INH | I1000 | 46.030 | 39.339 | −11.250 | 1.00 | 31.71 | I | C |
| ATOM | 2651 | C5 | INH | I1000 | 46.683 | 40.SS4 | −10.853 | 1.00 | 32.74 | I | C |
| ATOM | 2652 | C6 | INH | I1000 | 46.586 | 41.046 | −9.505 | 1.00 | 29.47 | I | C |
| ATOM | 2653 | C7 | INH | I1000 | 46.945 | 41.023 | −4.949 | 1.00 | 31.41 | I | C |
| ATOM | 2654 | C8 | INH | I1000 | 46.9S4 | 40.327 | −6.323 | 1.00 | 40.73 | I | C |
| ATOM | 2655 | N1 | INH | I1000 | 45.765 | 40.654 | −7.172 | 1.00 | 34.83 | I | N |
| ATOM | 2656 | C9 | INH | I1000 | 44.449 | 41.053 | −6.597 | 1.00 | 31.83 | I | C |
| ATOM | 2657 | C10 | INH | I1000 | 44.553 | 41.687 | −5.196 | 1.00 | 37.27 | I | C |
| ATOM | 2658 | C11 | INH | I1000 | 45.565 | 40.971 | −4.294 | 1.00 | 33.47 | I | C |
| ATOM | 2659 | C12 | INH | I1000 | 45.615 | 41.649 | −2.925 | 1.00 | 22.65 | I | C |
| ATOM | 2660 | C13 | INH | I1000 | 46.168 | 38.824 | −12.660 | 1.00 | 30.80 | I | C |
| ATOM | 2661 | O1 | INH | I1000 | 45.134 | 39.389 | −13.461 | 1.00 | 36.39 | I | O |
| ATOM | 2662 | C14 | INH | I1000 | 48.036 | 43.176 | −9.627 | 1.00 | 24.58 | I | C |
| ATOM | 2663 | N2 | INH | I1000 | 47.164 | 42.271 | −9.050 | 1.00 | 25.04 | I | N |
| ATOM | 2664 | O2 | INH | I1000 | 48.530 | 43.082 | −10.759 | 1.00 | 34.12 | I | O |
| ATOM | 2665 | C15 | INH | I1000 | 48.330 | 44.304 | −8.735 | 1.00 | 22.66 | I | C |
| ATOM | 2666 | C16 | INH | I1000 | 49.194 | 45.378 | −8.874 | 1.00 | 22.73 | I | C |
| ATOM | 2667 | C17 | INH | I1000 | 49.071 | 46.171 | −7.701 | 1.00 | 17.57 | I | C |
| ATOM | 2668 | C18 | INH | I1000 | 48.137 | 45.548 | −6.918 | 1.00 | 21.6S | I | C |
| ATOM | 2669 | O3 | INH | I1000 | 47.673 | 44.412 | −7.527 | 1.00 | 23.98 | I | O |
| ATOM | 2670 | C19 | INH | I1000 | 47.610 | 45.887 | −5.642 | 1.00 | 20.98 | I | C |
| ATOM | 2671 | N3 | INH | I1000 | 47.190 | 46.175 | −4.595 | 1.00 | 22.49 | I | N |
| END | | | | | | | | | | | |

REMARK c-fms (538-922 tie2 chimera) complexed with 1183648
REMARK refinement resolution: 500.0-1.8 A
REMARK starting r = 0.2383 free_r = 0.2832
REMARK final r = 0.2367 free_r = 0.2811
CRYST1 80.440 80.440 143.760 90.00 90.00 120.00 R 3
REMARK Written by CNX VERSION:2002

### REFERENCES

1. Sherr, C. J. (1988). The fms oncogene. Biochim Biophys Acta 948, 225-243.
2. Rettenmier, C. W., Roussel, M. F., and Sherr, C. J. (1988). The colony-stimulating factor 1 (CSF-1) receptor (c-fms proto-oncogene product) and its ligand. J Cell Sci Suppl 9, 27-44.
3. Wiktor-Jedrzejczak, W., Bartocci, A., Ferrante, A. W., Jr., Ahmed-Ansari, A., Sell, K. W., Pollard, J. W., and Stanley, E. R. (1990). Total absence of colony-stimulating factor 1 in the macrophage-deficient osteopetrotic (op/op) mouse. Proc Natl Acad Sci USA 87, 4828-4832.
4. Lin, E. Y., Nguyen, A. V., Russell, R. G., and Pollard, J. W. (2001). Colony-stimulating factor 1 promotes progression of mammary tumors to malignancy. J Exp Med 193, 727-740.
5. Campbell, I. K., Rich, M. J., Bischof, R. J., and Hamilton, J. A. (2000). The colony-stimulating factors and collagen-induced arthritis: exacerbation of disease by M-CSF and G-CSF and requirement for endogenous M-CSF. J Leukoc Biol 68, 144-150.
6. Bischof, R. J., Zafiropoulos, D., Hamilton, J. A., and Campbell, I. K. (2000). Exacerbation of acute inflammatory arthritis by the colony-stimulating factors CSF-1 and granulocyte macrophage (GM)-CSF: evidence of macrophage infiltration and local proliferation. Clin Exp Immunol 119, 361-367.
7. Kluger, H. M., Dolled-Filhart, M., Rodov, S., Kacinski, B. M., Camp, R. L., and Rimm, D. L. (2004). Macrophage colony-stimulating factor-1 receptor expression is associated with poor outcome in breast cancer by large cohort tissue microarray analysis. Clin Cancer Res 10, 173-177.
8. Kacinski, B. M. (1997). CSF-1 and its receptor in breast carcinomas and neoplasms of the female reproductive tract. Mol Reprod Dev 46, 71-74.
9. Kutza, J., Crim, L., Feldman, S., Hayes, M. P., Gruber, M., Beeler, J., and Clouse, K. A. (2000). Macrophage colony-stimulating factor antagonists inhibit replication of HIV-1 in human macrophages. J Immunol 164, 4955-4960.
10. Brünger, A. T., Adams, P. D., Clore, G. M., DeLano, W. L., Gros, P., Grosse-Kunstleve, R. W., Jiang, J.-S., Kuszewki, J., Nilges, M., Pannu, N. S., Read, R. J., Rice, L. M., Simonson, T., and Warren, G. L. (1998). Crystallography & NMR System: A new Software Suite for Macromolecular Structure Determination. Acta Cryst D54, 905-921.
11. Jones, T. A., Zou, J.-Y., Cowan, S. W., and Kjeldgaard, M. (1991). Improved methods for building protein models in electron density maps and the location of errors in these models. Acta Cryst A47, 110-119.
12. McTigue, M. A., Wickersham, J. A., Pinko, C., Showalter, R. E., Parast, C. V., Tempczyk-Russell, A., Gehring, M. R., Mroczkowski, B., Kan, C. C., Villafranca, J. E., and Appelt, K. (1999). Crystal structure of the kinase domain of human vascular endothelial growth factor receptor 2: a key enzyme in angiogenesis. Structure Fold Des 7, 319-330.
13. Hubbard, S. R., Wei, L., Ellis, L., and Hendrickson, W. A. (1994). Crystal structure of the tyrosine kinase domain of the human insulin receptor. Nature 372, 746-754.
14. Mohammadi, M., Schlessinger, J., and Hubbard, S. R. (1996). Structure of the FGF receptor tyrosine kinase domain reveals a novel autoinhibitory mechanism. Cell 86, 577-587.
15. Hubbard, S. R. (2002). Autoinhibitory mechanisms in receptor tyrosine kinases. Front Biosci 7, 330-340.
16. Feng, X., Takeshita, S., Namba, N., Wei, S., Teitelbaum, S. L., and Ross, F. P. (2002). Tyrosines 559 and 807 in the cytoplasmic tail of the macrophage colony-stimulating factor receptor play distinct roles in osteoclast differentiation and function. Endocrinology 143, 4868-4874.
17. van der Geer, P., and Hunter, T. (1991). Tyrosine 706 and 807 phosphorylation site mutants in the murine colony-stimulating factor-1 receptor are unaffected in their ability to bind or phosphorylate phosphatidylinositol-3 kinase but show differential defects in their ability to induce early response gene transcription. Mol Cell Biol 11, 4698-4709.
18. Roussel, M. F., Shurtleff, S. A., Downing, J. R., and Sherr, C. J. (1990). A point mutation at tyrosine-809 in the human colony-stimulating factor 1 receptor impairs mitogenesis without abrogating tyrosine kinase activity, association with phosphatidylinositol 3-kinase, or induction of c-fos and junB genes. Proc Natl Acad Sci USA 87, 6738-6742.
19. Joos, H., Trouliaris, S., Helftenbein, G., Niemann, H., and Tamura, T. (1996). Tyrosine phosphorylation of the juxtamembrane domain of the v-Fms oncogene product is required for its association with a 55-kDa protein. J Biol Chem 271, 24476-24481.
20. Alonso, G., Koegl, M., Mazurenko, N., and Courtneidge, S. A. (1995). Sequence requirements for binding of Src family tyrosine kinases to activated growth factor receptors. J Biol Chem 270, 9840-9848.
21. Griffith, J., Black, J., Faerman, C., Swenson, L., Wynn, M., Lu, F., Lippke, J., and Saxena, K. (2004). The structural basis for autoinhibition of FLT3 by the juxtamembrane domain. Mol Cell 13, 169-178.
22. Hubbard, S. R. (1997). Crystal structure of the activated insulin receptor tyrosine kinase in complex with peptide substrate and ATP analog. Embo J 16, 5572-5581.
23. van der Geer, P., and Hunter, T. (1993). Mutation of Tyr697, a GRB2-binding site, and Tyr721, a PI 3-kinase binding site, abrogates signal transduction by the murine CSF-1 receptor expressed in Rat-2 fibroblasts. Embo J 12, 5161-5172.
24. Mohammadi, M., McMahon, G., Sun, L., Tang, C., Hirth, P., Yeh, B. K., Hubbard, S. R., and Schlessinger, J. (1997). Structures of the tyrosine kinase domain of fibroblast growth factor receptor in complex with inhibitors. Science 276, 955-960.
25. DeLano, W. L. The PyMOL Molecular Graphics System (2002) DeLano Scientific, San Carlos, Calif., USA.

Although the present invention has been described in detail with reference to examples above, it is understood that various modifications can be made without departing from the spirit of the invention. All cited patents, published patent applications, publications and other documents cited in this application are herein incorporated by reference in their entirety.

We claim:

1. An isolated chimeric kinase receptor polypeptide, wherein said polypeptide comprises an ATP binding pocket linked to a substrate binding pocket by a kinase insert domain (KID), wherein said domain is heterologous to the ATP binding pocket or the substrate binding pocket.

2. The polypeptide of claim 1, wherein said ATP binding pocket and substrate binding pocket are c-fms.

3. The polypeptide of claim 1, wherein said heterologous KID is selected from the group consisting of FGFR1, tie2 and IRK.

4. The isolated chimeric kinase receptor polypeptide of claim 1, wherein said chimeric polypeptide comprises an amino acid sequence beginning at c-fms amino acid position 538 and continuing through c-fms amino acid position 922 wherein the native-c-fms KID is replaced with a KID sequence comprising a heterologous KID amino acid sequence beginning at c-fms amino acid positions 672-688.

5. The chimeric polypeptide of claim 4, wherein said heterologous KID is selected from the group consisting of FGFR1, tie2 and IRK.

6. The chimeric polypeptide of claim 4, wherein said polypeptide has an amino acid sequence having at least 95% amino acid sequence identity to a sequence selected from the group consisting of SEQ ID NO. 2 (FMS/FGFR1 chimera); SEQ ID NO. 4 (FMS/tie chimera) and SEQ. ID NO: 6 (FMS/irk chimera).

7. The chimeric polypeptide of claim 4 in crystalline form.

8. A crystal comprising a polypeptide of claim 4, or a fragment thereof.

9. The crystal of claim 8, comprising a crystal structure defined by one or more structure coordinates of c-fms amino acid residues Trp 550, Lys 586, Thr 587, Leu 588, Gly 589, Val 596, Glu 598, Ala 614, Val 615, Lys 616, Glu 633, Met 637, Leu 640, Ile 646, Val 647, Val 661, Thr 663, Glu 664, Tyr 665, Cys 666, Cys 667, Tyr 668, Gly 669, Asp 670, Asn 673, Arg 677, Cys 774, Ile 775, His 776, Arg 782, Asn 783, Leu 785, Ile 794, Gly 795, Asp 796, Phe 797, Gly 798, Leu 799, Ala 800, Arg 801, Asp 802 according to Tables 1, 2 or 3 or similar structure coordinates for said amino acids comprising a root mean square deviation of non-hydrogen atoms of less than about 1.5 Å when superimposed on the non-hydrogen atom positions of the corresponding atomic coordinates of Tables 1, 2 or 3.

10. The crystal of claim 9, comprising a crystal structure defined by one or more structure coordinates of c-fms amino acid residues Trp 550, Lys 586, Thr 587, Leu 588, Gly 589, Val 596, Glu 598, Ala 614, Val 615, Lys 616, Glu 633, Met 637, Leu 640, Ile 646, Val 647, Val 661, Thr 663, Glu 664, Tyr 665, Cys 666, Cys 667, Tyr 668, Gly 669, Asp 670, Asn 673, Arg 677, Cys 774, Ile 775, His 776, Arg 782, Asn 783, Leu 785, Ile 794, Gly 795, Asp 796, Phe 797, Gly 798, Leu 799, Ala 800, Arg 801, Asp 802 according to Tables 1, 2 or 3 or similar structure coordinates for said amino acids comprising a root mean square deviation of non-hydrogen atoms of less than about 0.75 Å when superimposed on the non-hydrogen atom positions of the corresponding atomic coordinates of Tables 1, 2 or 3.

11. The crystal of claim 8, comprising a crystal structure defined by one or more structure coordinates of c-fms amino acid residues Lys 586, Leu 588, Gly 589, Val 596, Glu 598, Ala 614, Lys 616, Val 647, Thr 663, Glu 664, Tyr 665, Cys 666, Cys 667, Tyr 668, Gly 669, Asp 670, Arg 677, Arg 782, Leu 785, Asp 796, Phe 797, Gly 798, Leu 799, Ala 800, Arg 801, Asp 802 according to Tables 1, 2 or 3 or similar structure coordinates for said amino acids comprising a root mean square deviation of non-hydrogen atoms of less than about 1.5 Å when superimposed on the non-hydrogen atom positions of the corresponding atomic coordinates of Tables 1, 2 or 3.

12. The crystal of claim 11, comprising a crystal structure defined by one or more structure coordinates of c-fms amino acid residues Lys 586, Leu 588, Gly 589, Val 596, Glu 598, Ala 614, Lys 616, Val 647, Thr 663, Glu 664, Tyr 665, Cys 666, Cys 667, Tyr 668, Gly 669, Asp 670, Arg 677, Arg 782, Leu 785, Asp 796, Phe 797, Gly 798, Leu 799, Ala 800, Arg 801, Asp 802 according to Tables 1, 2 or 3 or similar structure coordinates for said amino acids comprising a root mean square deviation of non-hydrogen atoms of less than about 0.75 Å when superimposed on the non-hydrogen atom positions of the corresponding atomic coordinates of Tables 1, 2 or 3.

13. The crystal of claim 8, comprising a crystal structure defined by structure coordinates of c-fms amino acid residues Lys 586, Leu 588, Gly 589, Val 596, Glu 598, Ala 614, Lys 616, Val 647, Thr 663, Glu 664, Tyr 665, Cys 666, Cys 667, Tyr 668, Gly 669, Asp 670, Arg 782, Asn 783, Leu 785, Asp 796, Phe 797, Leu 799, Ala 800, Arg 801 according to Tables 1, 2 or 3 or similar structure coordinates for said amino acids comprising a root mean square deviation of non-hydrogen atoms of less than about 1.5 Å when superimposed on the non-hydrogen atom positions of the corresponding atomic coordinates of Tables 1, 2 or 3.

14. The crystal of claim 13, comprising a crystal structure defined by structure coordinates of c-fms amino acid residues Lys 586, Leu 588, Gly 589, Val 596, Glu 598, Ala 614, Lys 616, Val 647, Thr 663, Glu 664, Tyr 665, Cys 666, Cys 667, Tyr 668, Gly 669, Asp 670, Arg 782, Asn 783, Leu 785, Asp 796, Phe 797, Leu 799, Ala 800, Arg 801 according to Tables 1, 2 or 3 or similar structure coordinates for said amino acids comprising a root mean square deviation of non-hydrogen atoms of less than about 0.75 Å when superimposed on the non-hydrogen atom positions of the corresponding atomic coordinates of Tables 1, 2 or 3.

15. The crystal of claim 8, further comprising a ligand, wherein said ligand is an ATP-binding pocket ligand.

16. The crystal of claim 15, wherein said ATP-binding pocket ligand is a small molecule inhibitor.

17. The crystal of claim 16, wherein said small molecule inhibitor is an arylamide compound or a derivative thereof.

18. The crystal of claim 16, wherein said small molecule inhibitor is a quinolone compound or a derivative thereof.

19. The crystal of claim 17, wherein said arylamide compound is 5-cyano-furan-2-carboxylic acid [5-hydroxymethyl-2-(4-methyl-piperidine-1-yl)-phenyl]-amide or derivative thereof.

20. The crystal of claim 18, wherein said quinolone compound is 6-Chloro-3-(3-methyl-isoxazol-5-yl)-4-phenyl-1H-quinolin-2-one or a derivative thereof.

21. The crystal of claim 16, wherein said crystal-ligand complex has a space group of R3. (Form I)

22. The crystal of claim 16, wherein said crystal-ligand complex has a space group of P2₁2₁2₁. (Form II)

23. The crystal of claim 21, wherein the crystal effectively diffracts X-rays for determination of atomic coordinates to a resolution of at least about 1.9 Å (Form I).

24. The crystal of claim 22, wherein the crystal effectively diffracts X-rays for determination of atomic coordinates to a resolution of at least about 3.0 Å (Form II).

25. The crystal of claim 23, comprising a unit cell having dimensions consisting of: a=81.07; b=81.07; c=144.67; alpha=90; beta=90; gamma=120.

26. The crystal of claim 24 comprising a unit cell having dimensions consisting of a 53.1; b=72.4; c=91.7; alpha=90; beta=90; gamma=90.

27. The crystal of claim 8, wherein said polypeptide comprises a peptide having at least 95% amino acid sequence identity to SEQ ID NO. 2 (FMS/FGFR1 chimera); SEQ ID NO. 4 (FMS/tie chimera) or SEQ ID NO: 6 (FMS/irk chimera).

28. The crystal of claim 27 wherein said crystal comprises a peptide having at least 95% sequence identity to SEQ ID NO. 2.

29. A crystal comprising SEQ ID NO: 2 comprising an atomic structure characterized by the coordinates of Tables 1, 2 or 3.

30. An isolated nucleic acid molecule encoding a polypeptide of claim 4.

31. A vector comprising a nucleic acid molecule of claim 30.

32. A host cell comprising a vector of claim 31.

33. A method of producing a polypeptide, comprising culturing the host cell of claim 32 under conditions in which the polypeptide is expressed.

34. A computer system comprising:
(a) a database containing information on the three dimensional structure of a crystal comprising a c-fms chimera, or a fragment or a target structural motif or derivative thereof, and a ligand, wherein said ligand is a small molecule inhibitor, stored on a computer readable storage medium; and,
(b) a user interface to view the information.

35. The computer system of claim 34, wherein the information comprises diffraction data obtained from a crystal comprising SEQ ID NO: 2, 4 or 6.

36. The computer system of claim 34, wherein the information comprises an electron density map of a crystal form comprising SEQ ID NO: 2, 4 or 6.

37. The computer system of claim 34, wherein the information comprises the structure coordinates of Tables 1, 2 or 3 or homologous structure coordinates for the amino acids of SEQ ID NO: 2 comprising a root mean square deviation of non-hydrogen atoms of less than about 1.5 Å when superimposed on the non-hydrogen atom positions of the corresponding atomic coordinates of Tables 1, 2 or 3.

38. The computer system of claim 37, wherein the information comprises structure coordinates for amino acid residues of SEQ ID NO: 2 comprising a root mean square deviation of non-hydrogen atoms of less than about 0.75 Å when superimposed on the non-hydrogen atom positions of the corresponding atomic coordinates of Tables 1, 2 or 3.

39. The computer system of claim 34, wherein the information comprises the structure coordinates for one or more amino acid residues Trp 550, Lys 586, Thr 587, Leu 588, Gly 589, Val 596, Glu 598, Ala 614, Val 615, Lys 616, Glu 633, Met 637, Leu 640, Ile 646, Val 647, Val 661, Thr 663, Glu 664, Tyr 665, Cys 666, Cys 667, Tyr 668, Gly 669, Asp 670, Asn 673, Arg 677, Cys 774, Ile 775, His 776, Arg 782, Asn 783, Leu 785, Ile 794, Gly 795, Asp 796, Phe 797, Gly 798, Leu 799, Ala 800, Arg 801, Asp 802 according to Tables 1, 2 or 3 or similar structure coordinates for said amino acids comprising a root mean square deviation of non-hydrogen atoms of less than about 1.5 Å when superimposed on the non-hydrogen atom positions of the corresponding atomic coordinates of Tables 1, 2 or 3.

40. The computer system of claim 39, wherein the information further comprises the structure coordinates for one or more amino acid residues Trp 550, Lys 586, Thr 587, Leu 588, Gly 589, Val 596, Glu 598, Ala 614, Val 615, Lys 616, Glu 633, Met 637, Leu 640, Ile 646, Val 647, Val 661, Thr 663, Glu 664, Tyr 665, Cys 666, Cys 667, Tyr 668, Gly 669, Asp 670, Asn 673, Arg 677, Cys 774, Ile 775, His 776, Arg 782, Asn 783, Leu 785, Ile 794, Gly 795, Asp 796, Phe 797, Gly 798, Leu 799, Ala 800, Arg 801, Asp 802 according to Tables 1, 2 or 3 or similar structure coordinates for said amino acids comprising a root mean square deviation of non-hydrogen atoms of less than about 0.75 Å when superimposed on the non-hydrogen atom positions of the corresponding atomic coordinates of Tables 1, 2 or 3.

41. The computer system of claim 34, comprising a crystal structure defined by structure coordinates of one or more c-fms amino acid residues Lys 586, Leu 588, Gly 589, Val 596, Glu 598, Ala 614, Lys 616, Val 647, Thr 663, Glu 664, Tyr 665, Cys 666, Cys 667, Tyr 668, Gly 669, Asp 670, Arg 677, Arg 782, Leu 785, Asp 796, Phe 797, Gly 798, Leu 799, Ala 800, Arg 801, Asp 802 according to Tables 1, 2 or 3 or similar structure coordinates for said amino acids comprising a root mean square deviation of non-hydrogen atoms of less than about 1.5 Å when superimposed on the non-hydrogen atom positions of the corresponding atomic coordinates of Tables 1, 2 or 3.

42. The computer system of claim 41, comprising a crystal structure defined by structure coordinates of one or more c-fms amino acid residues Lys 586, Leu 588, Gly 589, Val 596, Glu 598, Ala 614, Lys 616, Val 647, Thr 663, Glu 664, Tyr 665, Cys 666, Cys 667, Tyr 668, Gly 669, Asp 670, Arg 677, Arg 782, Leu 785, Asp 796, Phe 797, Gly 798, Leu 799, Ala 800, Arg 801, Asp 802 according to Tables 1, 2 or 3 or similar structure coordinates for said amino acids comprising a root mean square deviation of non-hydrogen atoms of less than about 0.75 Å when superimposed on the non-hydrogen atom positions of the corresponding atomic coordinates of Tables 1, 2 or 3.

43. The computer system of claim 34, comprising a crystal structure defined by structure coordinates of c-fms amino acid residues Lys 586, Leu 588, Gly 589, Val 596, Glu 598, Ala 614, Lys 616, Val 647, Thr 663, Glu 664, Tyr 665, Cys 666, Cys 667, Tyr 668, Gly 669, Asp 670, Arg 782, Asn 783, Leu 785, Asp 796, Phe 797, Leu 799, Ala 800, Arg 801 according to Tables 1, 2 or 3: or similar structure coordinates for said amino acids comprising a root mean square deviation of non-hydrogen atoms of less than about 1.5 Å when superimposed on the non-hydrogen atom positions of the corresponding atomic coordinates of Tables 1, 2 or 3.

44. The computer system of claim 43, comprising a crystal structure defined by structure coordinates of c-fms amino acid residues Lys 586, Leu 588, Gly 589, Val 596, Glu 598, Ala 614, Lys 616, Val 647, Thr 663, Glu 664, Tyr 665, Cys 666, Cys 667, Tyr 668, Gly 669, Asp 670, Arg 782, Asn 783, Leu 785, Asp 796, Phe 797, Leu 799, Ala 800, Arg 801 according to Tables 1, 2 or 3 or similar structure coordinates for said amino acids comprising a root mean square deviation of non-hydrogen atoms of less than about 0.75 Å when superimposed on the non-hydrogen atom positions of the corresponding atomic coordinates of Tables 1, 2 or 3.

45. A method of evaluating the potential of an agent to associate with c-fms chimeric polypeptides comprising:
(a) exposing the c-fms chimera to the agent; and
(b) detecting the association of said agent to one or more c-fms amino acid residues selected from the group consisting of
(i) Trp 550, Lys 586, Thr 587, Leu 588, Gly 589, Val 596, Glu 598, Ala 614, Val 615, Lys 616, Glu 633, Met 637, Leu 640, Ile 646, Val 647, Val 661, Thr 663, Glu 664, Tyr 665, Cys 666, Cys 667, Tyr 668, Gly 669, Asp 670, Asn 673, Arg 677, Cys 774, Ile 775, His 776, Arg 782, Asn 783, Leu 785, Ile 794, Gly 795, Asp 796, Phe 797, Gly 798, Leu 799, Ala 800, Arg 801, Asp 802;
(ii) Lys 586, Leu 588, Gly 589, Val 596, Glu 598, Ala 614, Lys 616, Val 647, Thr 663, Glu 664, Tyr 665, Cys 666, Cys 667, Tyr 668, Gly 669, Asp 670, Arg 677, Arg 782, Leu 785, Asp 796, Phe 797, Gly 798, Leu 799, Ala 800, Arg 801, Asp 802; and,
(iii) Lys 586, Leu 588, Gly 589, Val 596, Glu 598, Ala 614, Lys 616, Val 647, Thr 663, Glu 664, Tyr 665, Cys 666, Cys 667, Tyr 668, Gly 669, Asp 670, Arg 782, Asn 783, Leu 785, Asp 796, Phe 797, Leu 799, Ala 800, Arg 801 thereby evaluating the potential.

46. The method of claim 45, wherein the agent is a virtual compound.

47. A method of evaluating the potential of an agent to associate with a polypeptide of claim 1, comprising:
(a) exposing the polypeptide to the agent; and
(b) detecting the level of association of the agent to the polypeptide,
thereby evaluating the potential of the agent to associate with the polypeptide.

48. The method of claim 47, wherein the agent is a virtual compound.

49. The method of claim 47, wherein step (a) comprises comparing the atomic structure of the compound to the three dimensional structure of a c-fms chimeric polypeptide.

50. The method of claim 49, wherein the comparing comprises employing a computational means to perform a fitting operation between the compound and at least one binding site of a c-fms chimera.

51. The method of claim 50, wherein the binding site is defined by one or more structure coordinates for amino acids Trp 550, Lys 586, Thr 587, Leu 588, Gly 589, Val 596, Glu 598, Ala 614, Val 615, Lys 616, Glu 633, Met 637, Leu 640, Ile 646, Val 647, Val 661, Thr 663, Glu 664, Tyr 665, Cys 666, Cys 667, Tyr 668, Gly 669, Asp 670, Asn 673, Arg 677, Cys 774, Ile 775, His 776, Arg 782, Asn 783, Leu 785, Ile 794, Gly 795, Asp 796, Phe 797, Gly 798, Leu 799, Ala 800, Arg 801, Asp 802 according to Tables 1, 2 or 3 or similar structure coordinates for said amino acids of a c-fms chimera comprising a root mean square deviation of non-hydrogen atoms of less than about 1.5 Å when superimposed on the non-hydrogen atom positions of the corresponding atomic coordinates of Tables 1, 2 or 3.

52. The method of claim 51, wherein the binding site is defined by one or more structure coordinates for amino acids Trp 550, Lys 586, Thr 587, Leu 588, Gly 589, Val 596, Glu 598, Ala 614, Val 615, Lys 616, Glu 633, Met 637, Leu 640, Ile 646, Val 647, Val 661, Thr 663, Glu 664, Tyr 665, Cys 666, Cys 667, Tyr 668, Gly 669, Asp 670, Asn 673, Arg 677, Cys 774, Ile 775, His 776, Arg 782, Asn 783, Leu 785, Ile 794, Gly 795, Asp 796, Phe 797, Gly 798, Leu 799, Ala 800, Arg 801, Asp 802 according to Tables 1, 2 or 3 or similar structure coordinates for said amino acids of a c-fms chimera comprising a root mean square deviation of non-hydrogen atoms of less than about 0.75 Å when superimposed on the non-hydrogen atom positions of the corresponding atomic coordinates of Tables 1, 2 or 3.

53. The method of claim 50, comprising a crystal structure defined by one or more structure coordinates of c-fms amino acid residues Lys 586, Leu 588, Gly 589, Val 596, Glu 598, Ala 614, Lys 616, Val 647, Thr 663, Glu 664, Tyr 665, Cys 666, Cys 667, Tyr 668, Gly 669, Asp 670, Arg 677, Arg 782, Leu 785, Asp 796, Phe 797, Gly 798, Leu 799, Ala 800, Arg 801, Asp 802 according to Tables 1, 2 or 3 or similar structure coordinates for said amino acids comprising a root mean square deviation of non-hydrogen atoms of less than about 1.5 Å when superimposed on the non-hydrogen atom positions of the corresponding atomic coordinates of Tables 1, 2 or 3.

54. The method of claim 53, comprising a crystal structure defined by one or more structure coordinates of c-fms amino acid residues Lys 586, Leu 588, Gly 589, Val 596, Glu 598, Ala 614, Lys 616, Val 647, Thr 663, Glu 664, Tyr 665, Cys 666, Cys 667, Tyr 668, Gly 669, Asp 670, Arg 677, Arg 782, Leu 785, Asp 796, Phe 797, Gly 798, Leu 799, Ala 800, Arg 801, Asp 802 according to Tables 1, 2 or 3 or similar structure coordinates for said amino acids comprising a root mean square deviation of non-hydrogen atoms of less than about 0.75 Å when superimposed on the non-hydrogen atom positions of the corresponding atomic coordinates of Tables 1, 2 or 3.

55. The method of claim 50, comprising a crystal structure defined by structure coordinates of c-fms amino acid residues Lys 586, Leu 588, Gly 589, Val 596, Glu 598, Ala 614, Lys 616, Val 647, Thr 663, Glu 664, Tyr 665, Cys 666, Cys 667, Tyr 668, Gly 669, Asp 670, Arg 782, Asn 783, Leu 785, Asp 796, Phe 797, Leu 799, Ala 800, Arg 801 according to Tables 1, 2 or 3 or similar structure coordinates for said amino acids comprising a root mean square deviation of non-hydrogen atoms of less than about 1.5 Å when superimposed on the non-hydrogen atom positions of the corresponding atomic coordinates of Tables 1, 2 or 3.

56. The method of claim 55, comprising a crystal structure defined by structure coordinates of c-fms amino acid residues Lys 586, Leu 588, Gly 589, Val 596, Glu 598, Ala 614, Lys 616, Val 647, Thr 663, Glu 664, Tyr 665, Cys 666, Cys 667, Tyr 668, Gly 669, Asp 670, Arg 782, Asn 783, Leu 785, Asp 796, Phe 797, Leu 799, Ala 800, Arg 801 according to Tables 1, 2 or 3 or similar structure coordinates for said amino acids comprising a root mean square deviation of non-hydrogen atoms of less than about 0.75 Å when superimposed on the non-hydrogen atom positions of the corresponding atomic coordinates of Tables 1, 2 or 3.

57. The method of claim 47, wherein the agent is exposed to a crystalline c-fms chimera and the detecting of step (b) comprises determining the three dimensional structure of the agent-c-fms chimera complex.

58. A method of identifying a potential agonist or antagonist against a c-fms chimera comprising:
(a) employing the three dimensional structure of the c-fms chimera cocrystallized with a small molecule inhibitor to design or select said potential agonist or antagonist.

59. The method of claim 58, wherein the three dimensional structure corresponds to the atomic structure characterized by the coordinates of Tables 1, 2 or 3 or similar structure coordinates for said c-fms chimera comprising a root mean square deviation of non-hydrogen atoms of less than about 1.5 Å when superimposed on the non-hydrogen atom positions of the corresponding atomic coordinates of Tables 1, 2 or 3.

60. The method of claim 59, further comprising the steps of: (b) synthesizing the potential agonist or antagonist; and (c) contacting the potential agonist or antagonist with a chimeric c-fms polypeptide.

61. A method of locating the attachment site of an inhibitor to a c-fms chimeric polypeptide, comprising:
(a) obtaining X-ray diffraction data for a crystal of a chimeric c-fms polypeptide;
(b) obtaining X-ray diffraction data for a complex of a chimeric c-fms polypeptide and the inhibitor;
(c) subtracting the X-ray diffraction data obtained in step (a) from the X-ray diffraction data obtained in step (b) to obtain the difference in the X-ray diffraction data;
(d) obtaining phases that correspond to X-ray diffraction data obtained in step (a);
(e) utilizing the phases obtained in step (d) and the difference in the X-ray diffraction data obtained in step (c) to compute a difference Fourier image of the inhibitor; and,
(f) locating the attachment site of the inhibitor based on the computations obtained in step (e).

62. A method of obtaining a modified inhibitor comprising:
(a) obtaining a crystal comprising a chimeric c-fms polypeptide and an inhibitor;
(b) obtaining the atomic coordinates of the crystal;
(c) using the atomic coordinates and one or more molecular modeling techniques to determine how to modify the interaction of the inhibitor with the chimeric c-fms polypeptide; and
(d) modifying the inhibitor based on the determinations obtained in step (c) to produce a modified inhibitor.

63. The method of claim 62, wherein said crystal comprises a peptide selected from the group consisting of: a peptide having SEQ ID NO: 2; a peptide having SEQ ID NO: 4 and a peptide having SEQ ID NO: 6.

64. A method of claim 62, wherein the one or more molecular modeling techniques are selected from the group consisting of graphic molecular modeling and computational chemistry.

65. The method of claim 62, wherein step (b) comprises detecting the interaction of the inhibitor to one or more amino acid residues selected from the group consisting of
(i) Trp 550, Lys 586, Thr 587, Leu 588, Gly 589, Val 596, Glu 598, Ala 614, Val 615, Lys 616, Glu 633, Met 637, Leu 640, Ile 646, Val 647, Val 661, Thr 663, Glu 664, Tyr 665, Cys 666, Cys 667, Tyr 668, Gly 669, Asp 670, Asn 673, Arg 677, Cys 774, Ile 775, His 776, Arg 782, Asn 783, Leu 785, Ile 794, Gly 795, Asp 796, Phe 797, Gly 798, Leu 799, Ala 800, Arg 801, Asp 802;
(ii) Lys 586, Leu 588, Gly 589, Val 596, Glu 598, Ala 614, Lys 616, Val 647, Thr 663, Glu 664, Tyr 665, Cys 666, Cys 667, Tyr 668, Gly 669, Asp 670, Arg 677, Arg 782, Leu 785, Asp 796, Phe 797, Gly 798, Leu 799, Ala 800, Arg 801, Asp 802; and,
(iii) Lys 586, Leu 588, Gly 589, Val 596, Glu 598, Ala 614, Lys 616, Val 647, Thr 663, Glu 664, Tyr 665, Cys 666, Cys 667, Tyr 668, Gly 669, Asp 670, Arg 782, Asn 783, Leu 785, Asp 796, Phe 797, Leu 799, Ala 800, Arg 801.

66. An inhibitor of a chimeric c-fms polypeptide identified by the method of claim 62.

67. An isolated protein fragment comprising a binding pocket or active site defined by one or more structure coordinates of chimeric c-fms amino acid-residues selected from the group consisting of
(i) Trp 550, Lys 586, Thr 587, Leu 588, Gly 589, Val 596, Glu 598, Ala 614, Val 615, Lys 616, Glu 633, Met 637, Leu 640, Ile 646, Val 647, Val 661, Thr 663, Glu 664, Tyr 665, Cys 666, Cys 667, Tyr 668, Gly 669, Asp 670, Asn 673, Arg 677, Cys 774, Ile 775, His 776, Arg 782, Asn 783, Leu 785, Ile 794, Gly 795, Asp 796, Phe 797, Gly 798, Leu 799, Ala 800, Arg 801, Asp 802;
(ii) Lys 586, Leu 588, Gly 589, Val 596, Glu 598, Ala 614, Lys 616, Val 647, Thr 663, Glu 664, Tyr 665, Cys 666, Cys 667, Tyr 668, Gly 669, Asp 670, Arg 677, Arg 782, Leu 785, Asp 796, Phe 797, Gly 798, Leu 799, Ala 800, Arg 801, Asp 802; and,
(iii) Lys 586, Leu 588, Gly 589, Val 596, Glu 598, Ala 614, Lys 616, Val 647, Thr 663, Glu 664, Tyr 665, Cys 666, Cys 667, Tyr 668, Gly 669, Asp 670, Arg 782, Asn 783, Leu 785, Asp 796, Phe 797, Leu 799, Ala 800, Arg 801.

68. An isolated fragment of claim 67 linked to a solid support.

69. An isolated nucleic acid molecule encoding the fragment of claim 68.

70. A vector comprising a nucleic acid molecule of claim 69.

71. A host cell comprising the vector of claim 70.

72. A method of producing a protein fragment, comprising culturing the host cell of claim 71 under conditions in which the fragment is expressed.

73. A method of screening for an agent that associates with a chimeric c-fms polypeptide, comprising:
(a) exposing a protein molecule fragment of claim 67 to the agent; and
(b) detecting the level of association of the agent to the fragment.

74. A kit comprising a protein molecule fragment of claim 67.

75. A method for the production of a crystal complex comprising a chimeric c-fms chimeric polypeptide-ligand comprising:
(a) contacting the chimeric c-fms polypeptide with said ligand in a suitable solution and,
(b) crystallizing said resulting complex of chimeric c-fms polypeptide-ligand from said solution.

76. The method of claim 75, wherein said polypeptide is a polypeptide having SEQ ID NO: 2, 4 or 6.

77. The method of claim 75, further comprising contacting the crystalline chimeric c-fms polypeptide-ligand complex with another ligand in a suitable solution to replace the bound ligand.

78. A method for the production of a crystal of claim 1 comprising crystallizing a peptide comprising a sequence selected from the group consisting of SEQ ID NO: 2, 4 or 6 with a potential inhibitor.

79. A method for identifying a potential inhibitor of a chimeric c-fms polypeptide comprising:
(a) using a three dimensional structure of a chimeric c-fms polypeptide as defined by atomic coordinates according to Tables 1, 2 or 3 or similar structure coordinates for said amino acids of a c-fms chimera comprising a root mean square deviation of non-hydrogen atoms of less than about 1.5 Å when superimposed on the non-hydrogen atom positions of the corresponding atomic coordinates of Tables 1, 2 or 3;
(b) replacing one or more chimeric c-fms polypeptide amino acids selected from the group consisting of
(i) Trp 550, Lys 586, Thr 587, Leu 588, Gly 589, Val 596, Glu 598, Ala 614, Val 615, Lys 616, Glu 633, Met 637, Leu 640, Ile 646, Val 647, Val 661, Thr 663, Glu 664, Tyr 665, Cys 666, Cys 667, Tyr 668, Gly 669, Asp 670, Asn 673, Arg 677, Cys 774, Ile 775, His 776, Arg 782, Asn 783, Leu 785, Ile 794, Gly 795, Asp 796, Phe 797, Gly 798, Lei 799, Ala 800, Arg 801, Asp 802;
(ii) Lys 586, Leu 588, Gly 589, Val 596, Glu 598, Ala 614, Lys 616, Val 647, Thr 663, Glu 664, Tyr 665, Cys 666, Cys 667, Tyr 668, Gly 669, Asp 670, Arg 677, Arg 782, Leu 785, Asp 796, Phe 797, Gly 798, Leu 799, Ala 800, Arg 801, Asp 802; and,
(iii) Lys 586, Leu 588, Gly 589, Val 596, Glu 598, Ala 614, Lys 616, Val 647, Thr 663, Glu 664, Tyr 665, Cys 666, Cys 667, Tyr 668, Gly 669, Asp 670, Arg 782, Asn 783, Leu 785, Asp 796, Phe 797, Leu 799, Ala 800, Arg 801 in said three-dimensional structure with a different amino acid to produce a modified three-dimensional structure; and,
(c) using said modified three-dimensional structure to design or select said potential inhibitor.

80. The method of claim 79, further comprising d) synthesizing said potential inhibitor.

81. The method of claim 79, further comprising e) contacting said potential inhibitor with said modified chimeric c-fms polypeptide in the presence of a ligand to test the ability of said potential inhibitor to inhibit a chimeric c-fms polypeptide or said modified chimeric c-fms polypeptide.

82. The method of claim 79 wherein said replacing one or more amino acid residues-further comprises replacing SEQ ID NO: 2 amino acid residues selected from the group consisting of Lys 586, Leu 588, Gly 589, Val 596, Glu 598, Ala 614, Lys 616, Val 647, Thr 663, Glu 664, Tyr 665, Cys 666, Cys 667, Tyr 668, Gly 669, Asp 670, Arg 782, Asn 783, Leu 785, Asp 796, Phe 797, Leu 799, Ala 800, Arg 801.

83. The method of claim 79, wherein said potential inhibitor is selected from a database.

84. The method of claim 79, wherein said potential inhibitor is designed de novo.

85. The method of claim 79, wherein said potential inhibitor is designed from a known inhibitor.

86. The method of claim 79, wherein said step of employing said modified three-dimensional structure to design or select said potential inhibitor comprises the steps of:
(a) identifying chemical entities or fragments capable of associating with a modified chimeric c-fms polypeptide; and
(b) assembling the identified chemical entities or fragments into a single molecule to provide the structure of said potential inhibitor.

87. The method of claim 79, wherein the potential inhibitor is a competitive inhibitor.

88. The method of claim 79, wherein said potential inhibitor is a non-competitive or uncompetitive inhibitor.

89. The method of claim 79, wherein said potential inhibitor is an irreversible inhibitor.

90. The inhibitor identified by the method of claim 79.


SEQUENCE LISTING (9 sequences)

SEQ ID NO: 1 - DNA, 999 bp, Homo sapiens
tacaagtataagcagaagcccaagtaccaggtccgctggaagatcatcgagagctatgagggcaacagttatactttcatcgaccccacgcagctgccttacaacgagaagtgggagttcccccggaacaacctgcagtttggtaagaccctcggagctggagcctttgggaaggtggtggaggccacggcctttggtctgggcaaggaggatgctgtcctgaaggtggctgtgaagatgctgaagtccacggcccatgctgatgagaaggaggccctcatgtccgagctgaagatcatgagccacctgggccagcacgagaacatcgtcaaccttctgggagcctgtacccatggaggccctgtactggtcatcacggagtactgttgctatggcgacctgctcaactttctgcgaaggaagaggcctcctgggctggagtactcttataaccccagccacaaccccgaggaacagctgtcttcccgggacctgcttcacttctccagccaagtagcccagggcatggccttcctcgcttccaagaattgcatccaccgggacgtggcagcgcgtaacgtgctgttgaccaatggtcatgtggccaagattggggacttcgggctggctagggacatcatgaatgactccaactacattgtcaagggcaatgcccgcctgcctgtgaagtggatggccccagagagcatctttgactgtgtctacacggttcagagcgacgtctggtcctatggcatcctcctctgggagatcttctcacttgggctgaatccctaccctggcatcctggtgaacagcaagttctataaactggtgaaggatggataccaaatggcccagcctgcatttgccccaaagaatatatacagcatcatgcaggcctgctgggccttggagcccacccacagacccaccttccagcagatctgctccttccttcaggagcaggcccaagaggacaggagagagcgggactaa

SEQ ID NO: 2 - protein, 348 aa, Homo sapiens
MHHHHHHENLYFQGVDYKYKQKPKYQVRWKIIESYEGNSYTFIDPTQLPYNEKWEFPRNNLQFGKTLGAGAFGKVVEATAFGLGKEDAVLKVAVKMLKSTAHADEKEALMSELKIMSHLGQHENIVNLLGACTHGGPVLVITEYCCYGDLLNFLRRKRPPGLEYSYNPSHNPEEQLSSRDLLHFSSQVAQGMAFLASKNCIHRDVAARNVLLTNGHVAKIGDFGLARDIMNDSNYIVKGNARLPVKWMAPESIFDCVYTVQSDVWSYGILLWEIFSLGLNPYPGILVNSKFYKLVKDGYQMAQPAFAPKNIYSIMQACWALEPTHRPTFQQICSFLQEQAQEDRRERD

SEQ ID NO: 3 - DNA, 993 bp, Homo sapiens
tacaagtataagcagaagcccaagtaccaggtccgctggaagatcatcgagagctatgagggcaacagttatactttcatcgaccccacgcagctgccttacaacgagaagtgggagttcccccggaacaacctgcagtttggtaagaccctcggagctggagcctttgggaaggtggtggaggccacggcctttggtctgggcaaggaggatgctgtcctgaaggtggctgtgaagatgctgaagtccacggcccatgctgatgagaaggaggccctcatgtccgagctgaagatcatgagccacctgggccagcacgagaacatcgtcaaccttctgggagcctgtacccatggaggccctgtactggtcatcacggagtactgttgctatggcgacctgctcaactttctgcgaaggaagagccgtgtgctggagacggacccagcatttgccattgccaatagcaccgcgtccacacgggacctgcttcacttctccagccaagtagcccagggcatggccttcctcgcttccaagaattgcatccaccgggacgtggcagcgcgtaacgtgctgttgaccaatggtcatgtggccaagattggggacttcgggctggctagggacatcatgaatgactccaactacattgtcaagggcaatgcccgcctgcctgtgaagtggatggccccagagagcatctttgactgtgtctacacggttcagagcgacgtctggtcctatggcatcctcctctgggagatcttctcacttgggctgaatccctaccctggcatcctggtgaacagcaagttctataaactggtgaaggatggataccaaatggcccagcctgcatttgccccaaagaatatatacagcatcatgcaggcctgctgggccttggagcccacccacagacccaccttccagcagatctgctccttccttcaggagcaggcccaagaggacaggagagagcgggactaa

SEQ ID NO: 4 - protein, 346 aa, Homo sapiens
MHHHHHHENLYFQGVDYKYKQKPKYQVRWKIIESYEGNSYTFIDPTQLPYNEKWEFPRNNLQFGKTLGAGAFGKVVEATAFGLGKEDAVLKVAVKMLKSTAHADEKEALMSELKIMSHLGQHENIVNLLGACTHGGPVLVITEYCCYGDLLNFLRRKSRVLETDPAFAIANSTASTRDLLHFSSQVAQGMAFLASKNCIHRDVAARNVLLTNGHVAKIGDFGLARDIMNDSNYIVKGNARLPVKWMAPESIFDCVYTVQSDVWSYGILLWEIFSLGLNPYPGILVNSKFYKLVKDGYQMAQPAFAPKNIYSIMQACWALEPTHRPTFQQICSFLQEQAQEDRRERD

SEQ ID NO: 5 - DNA, 981 bp, Homo sapiens
tacaagtataagcagaagcccaagtaccaggtccgctggaagatcatcgagagctatgagggcaacagttatactttcatcgaccccacgcagctgccttacaacgagaagtgggagttcccccggaacaacctgcagtttggtaagaccctcggagctggagcctttgggaaggtggtggaggccacggcctttggtctgggcaaggaggatgctgtcctgaaggtggctgtgaagatgctgaagtccacggcccatgctgatgagaaggaggccctcatgtccgagctgaagatcatgagccacctgggccagcacgagaacatcgtcaaccttctgggagcctgtacccatggaggccctgtactggtcatcacggagtactgttgctatggcgacctgctcaactttctgcgaaggaagtctctgcggccagaggctgagaataatcctggccgccctccccctacccgggacctgcttcacttctccagccaagtagcccagggcatggccttcctcgcttccaagaattgcatccaccgggacgtggcagcgcgtaacgtgctgttgaccaatggtcatgtggccaagattggggacttcgggctggctagggacatcatgaatgactccaactacattgtcaagggcaatgcccgcctgcctgtgaagtggatggccccagagagcatctttgactgtgtctacacggttcagagcgacgtctggtcctatggcatcctcctctgggagatcttctcacttgggctgaatccctaccctggcatcctggtgaacagcaagttctataaactggtgaaggatggataccaaatggcccagcctgcatttgccccaaagaatatatacagcatcatgcaggcctgctgggccttggagcccacccacagacccaccttccagcagatctgctccttccttcaggagcaggcccaagaggacaggagagagcgggac

SEQ ID NO: 6 - protein, 343 aa, Homo sapiens
MHHHHHHENLYFQGVDYKYKQKPKYQVRWKIIESYEGNSYTFIDPTQLPYNEKWEFPRNNLQFGKTLGAGAFGKVVEATAFGLGKEDAVLKVAVKMLKSTAHADEKEALMSELKIMSHLGQHENIVNLLGACTHGGPVLVITEYCCYGDLLNFLRRKSLRPEAENNPGRPPPTRDLLHFSSQVAQGMAFLASKNCIHRDVAARNVLLTNGHVAKIGDFGLARDIMNDSNYIVKGNARLPVKWMAPESIFDCVYTVQSDVWSYGILLWEIFSLGLNPYPGILVNSKFYKLVKDGYQMAQPAFAPKNIYSIMQACWALEPTHRPTFQQICSFLQEQAQEDRRERD

SEQ ID NO: 7 - DNA, 3985 bp, Homo sapiens
gaagggcagacagagtgtccaaaagcgtgagagcacgaagtgaggagaaggtggagaagagagaagaggaagaggaagaggaagagaggaagcggagggaactgcggccaggctaaaaggggaagaagaggatcagcccaaggaggaggaagaggaaaacaagacaaacagccagtgcagaggagaggaacgtgtgtccagtgtcccgatccctgcggagctagtagctgagagctctgtgccctgggcaccttgcagccctgcacctgcctgccacttccccaccgaggccatgggcccaggagttctgctgctcctgctggtggccacagcttggcatggtcagggaatcccagtgatagagcccagtgtccctgagctggtcgtgaagccaggagcaacggtgaccttgcgatgtgtgggcaatggcagcgtggaatgggatggccccccatcacctcactggaccctgtactctgatggctccagcagcatcctcagcaccaacaacgctaccttccaaaacacggggacctatcgctgcactgagcctggagaccccctgggaggcagcgccgccatccacctctatgtcaaagaccctgcccggccctggaacgtgctagcacaggaggtggtcgtgttcgaggaccaggacgcactactgccctgtctgctcacagacccggtgctggaagcaggcgtctcgctggtgcgtgtgcgtggccggcccctcatgcgccacaccaactactccttctcgccctggcatggcttcaccatccacagggccaagttcattcagagccaggactatcaatgcagtgccctgatgggtggcaggaaggtgatgtccatcagcatccggctgaaagtgcagaaagtcatcccagggcccccagccttgacactggtgcctgcagagctggtgcggattcgaggggaggctgcccagatcgtgtgctcagccagcagcgttgatgttaactttgatgtcttcctccaacacaacaacaccaagctcgcaatccctcaacaatctgactttcataataaccgttaccaaaaagtcctgaccctcaacctcgatcaagtagatttccaacatgccggcaactactcctgcgtggccagcaacgtgcagggcaagcactccacctccatgttcttccgggtggtagagagtgcctacttgaacttgagctctgagcagaacctcatccaggaggtgaccgtgggggaggggctcaacctcaaagtcatggtggaggcctacccaggcctgcaaggttttaactggacctacctgggacccttttctgaccaccagcctgagcccaagcttgctaatgctaccaccaaggacacatacaggcacaccttcaccctctctctgccccgcctgaagccctctgaggctggccgctactccttcctggccagaaacccaggaggctggagagctctgacgtttgagctcacccttcgataccccccagaggtaagcgtcatatggacattcatcaacggctctggcacccttttgtgtgctgcctctgggtacccccagcccaacgtgacatggctgcagtgcagtggccacactgataggtgtgatgaggcccaagtgctgcaggtctgggatgacccataccctgaggtcctgagccaggagcccttccacaaggtgacggtgcagagcctgctgactgttgagaccttagagcacaaccaaacctacgagtgcagggcccacaacagcgtggggagtggctcctgggccttcatacccatctctgcaggagcccacacgcatcccccggatgagttcctcttcacaccagtggtggtcgcctgcatgtccatcatggccttgctgctgctgctgctcctgctgctattgtacaagtataagcagaagcccaagtaccaggtccgctggaagatcatcgagagctatgagggcaacagttatactttcatcgaccccacgcagctgccttacaacgagaagtgggagttcccccggaacaacctgcagtttggtaagaccctcggagctggagcctttgggaaggtggtggaggccacggcctttggtctgggcaaggaggatgctgtcctgaaggtggctgtgaagatgctgaagtccacggcccatgctgatgagaaggaggccctcatgtccgagctgaagatcatgagccacctgggccagcacgagaacatcgtcaaccttctgggagcctgtacccatggaggccctgtactggtcatcacggagtactgttgctatggcgacctgctcaactttctgcgaaggaaggctgaggccatgctgggacccagcctgagccccggccaggaccccgagggaggcgtcgactataagaacatccacctcgagaagaaatatgtccgcagggacagtggcttctccagccagggtgtggacacctatgtggagatgaggcctgtctccacttcttcaaatgactccttctctgagcaagacctggacaaggaggatggacggcccctggagctccgggacctgcttcacttctccagccaagtagcccagggcatggccttcctcgcttccaagaattgcatccaccgggacgtggcagcgcgtaacgtgctgttgaccaatggtcatgtggccaagattggggacttcgggctggctagggacatcatgaatgactccaactacattgtcaagggcaatgcccgcctgcctgtgaagtggatggccccagagagcatctttgactgtgtctacacggttcagagcgacgtctggtcctatggcatcctcctctgggagatcttctcacttgggctgaatccctaccctggcatcctggtgaacagcaagttctataaactggtgaaggatggataccaaatggcccagcctgcatttgccccaaagaatatatacagcatcatgcaggcctgctgggccttggagcccacccacagacccaccttccagcagatctgctccttccttcaggagcaggcccaagaggacaggagagagcgggactataccaatctgccgagcagcagcagaagcggtggcagcggcagcagcagcagtgagctggaggaggagagctctagtgagcacctgacctgctgcgagcaaggggatatcgcccagcccttgctgcagcccaacaactatcagttctgctgaggagttgacgacagggagtaccactctcccctcctccaaacttcaactcctccatggatggggcgacacggggagaacatacaaactctgccttcggtcatttcactcaacagctcggcccagctctgaaacttgggaaggtgagggattcaggggaggtcagaggatcccacttcctgagcatgggccatcactgccagtcaggggctgggggctgagccctcacccccccctcccctactgttctcatggtgttggcctcgtgtttgctatgccaactagtagaaccttctttcctaatccccttatcttcatggaaatggactgactttatgcctatgaagtccccaggagctacactgatactgagaaaaccaggctctttggggctagacagactggcagagagtgagatctccctctctgagaggagcagcagatgctcacagaccacactcagctcaggccccttggagcaggatggctcctctaagaatctcacaggacctcttagtctctgccctatacgccgccttcactccacagcctcacccctcccacccccatactggtactgctgtaatgagccaagtggcagctaaaagttgggggtgttctgcccagtcccgtcattctgggctagaaggcaggggaccttggcatgtggctggccacaccaagcaggaagcacaaactcccccaagctgactcatcctaactaacagtcacgccgtgggatgtctctgtccacattaaactaacagcattaatgca

SEQ ID NO: 8 - protein, 972 aa, Homo sapiens
MGPGVLLLLLVATAWHGQGIPVIEPSVPELVVKPGATVTLRCVGNGSVEWDGPPSPHWTLYSDGSSSILSTNNATFQNTGTYRCTEPGDPLGGSAAIHLYVKDPARPWNVLAQEVVVFEDQDALLPCLLTDPVLEAGVSLVRVRGRPLMRHTNYSFSPWHGFTIHRAKFIQSQDYQCSALMGGRKVMSISIRLKVQKVIPGPPALTLVPAELVRIRGEAAQIVCSASSVDVNFDVFLQHNNTKLAIPQQSDFHNNRYQKVLTLNLDQVDFQHAGNYSCVASNVQGKHSTSMFFRVVESAYLNLSSEQNLIQEVTVGEGLNLKVMVEAYPGLQGFNWTYLGPFSDHQPEPKLANATTKDTYRHTFTLSLPRLKPSEAGRYSFLARNPGGWRALTFELTLRYPPEVSVIWTFINGSGTLLCAASGYPQPNVTWLQCSGHTDRCDEAQVLQVWDDPYPEVLSQEPFHKVTVQSLLTVETLEHNQTYECRAHNSVGSGSWAFIPISAGAHTHPPDEFLFTPVVVACMSIMALLLLLLLLLLYKYKQKPKYQVRWKIIESYEGNSYTFIDPTQLPYNEKWEFPRNNLQFGKTLGAGAFGKVVEATAFGLGKEDAVLKVAVKMLKSTAHADEKEALMSELKIMSHLGQHENIVNLLGACTHGGPVLVITEYCCYGDLLNFLRRKAEAMLGPSLSPGQDPEGGVDYKNIHLEKKYVRRDSGFSSQGVDTYVEMRPVSTSSNDSFSEQDLDKEDGRPLELRDLLHFSSQVAQGMAFLASKNCIHRDVAARNVLLTNGHVAKIGDFGLARDIMNDSNYIVKGNARLPVKWMAPESIFDCVYTVQSDVWSYGILLWEIFSLGLNPYPGILVNSKFYKLVKDGYQMAQPAFAPKNIYSIMQACWALEPTHRPTFQQICSFLQEQAQEDRRERDYTNLPSSSRSGGSGSSSSELEEESSSEHLTCCEQGDIAQPLLQPNNYQFC

SEQ ID NO: 9 - protein, 14 aa, Artificial Sequence
  Description of Artificial Sequence Synthetic peptide
SYEGNSYTFIDPTQ